SEQUENCE LISTING (21 sequences)

SEQ ID NO: 1 - protein, 107 aa, synthetic construct
  REGION 1..107: note = Synthetic
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 9 aa, synthetic construct
  REGION 1..9: note = Synthetic
IEVMYPPPY

SEQ ID NO: 3 - protein, 7 aa, Simian virus 40
PKKKRKV

SEQ ID NO: 4 - protein, 10 aa, Simian virus 40
PKKKRKVEAS

SEQ ID NO: 5 - protein, 16 aa, synthetic construct
  REGION 1..16: note = Synthetic
KRPAATKKAGQAKKKK

SEQ ID NO: 6 - protein, 9 aa, Homo sapiens
PAAKRVKLD

SEQ ID NO: 7 - protein, 11 aa, Homo sapiens
RQRRNELKRSP

SEQ ID NO: 8 - protein, 38 aa, Homo sapiens
NQSSNFGPMKGGNFGGRSSGPYGGGGQYFAKPRNQGGY

SEQ ID NO: 9 - protein, 42 aa, Homo sapiens
RMRIZFKNKGKDTAELRRRRVEVSVELRKAKKDEQILKRRNV

SEQ ID NO: 10 - protein, 8 aa, Homo sapiens
VSRKRPRP

SEQ ID NO: 11 - protein, 8 aa, Homo sapiens
PPKKARED

SEQ ID NO: 12 - protein, 8 aa, Homo sapiens
PQPKKKPL

SEQ ID NO: 13 - protein, 12 aa, Mus sp.
SALIKKKKKMAP

SEQ ID NO: 14 - protein, 5 aa, Influenza virus
DRLRR

SEQ ID NO: 15 - protein, 7 aa, Influenza virus
PKQKKRK

SEQ ID NO: 16 - protein, 10 aa, Hepatitis D virus
RKLKKKIKKL

SEQ ID NO: 17 - protein, 10 aa, Mus sp.
REKKKFLKRR

SEQ ID NO: 18 - protein, 20 aa, Homo sapiens
KRKGDEVDGVDEVAKKKSKK

SEQ ID NO: 19 - protein, 17 aa, Homo sapiens
RKCLQAGMNLEARKTKK

SEQ ID NO: 20 - protein, 288 aa, synthetic construct
  REGION 1..288: note = Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 21 - protein, 183 aa, synthetic construct
  REGION 1..183: note = Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

(19) United States
(12) Patent Application Publication
(10) Pub. No.: US 2024/0401150 A1
(43) Pub. Date: Dec. 5, 2024

(54) PANCREATIC DUCTAL ADENOCARCINOMA SIGNATURES AND USES THEREOF

(71) Applicants: The Broad Institute, Inc., Cambridge, MA (US); Massachusetts Institute of Technology, Cambridge, MA (US); THE GENERAL HOSPITAL CORPORATION, Boston, MA (US)

(72) Inventors: William HWANG, Boston, MA (US); Jimmy GUO, Cambridge, MA (US); Tyler JACKS, Cambridge, MA (US); Aviv REGEV, Cambridge, MA (US); Karthik JAGADEESH, Cambridge, MA (US); Hannah HOFFMAN, Cambridge, MA (US)

(73) Assignees: The Broad Institute, Inc., Cambridge, MA (US); Massachusetts Institute of Technology, Cambridge, MA (US); THE GENERAL HOSPITAL CORPORATION, Boston, MA (US)

(21) Appl. No.: 18/813,710

(22) Filed: Aug. 23, 2024

Related U.S. Application Data
(63) Continuation of application No. PCT/US2023/063203, filed on Feb. 24, 2023.
(60) Provisional application No. 63/313,596, filed on Feb. 24, 2022.

(51) Int. Cl. C12Q 1/6886; G01N 33/50

(52) U.S. Cl. CPC C12Q 1/6886; G01N 33/5023; C12Q 2600/112; C12Q 2600/118; G01N 2500/10

(57) ABSTRACT

Described herein are pancreatic ductal adenocarcinoma (PDAC) signatures and methods of detecting the same in a sample from a subject. Also described herein, are methods of methods of diagnosing, prognosing, and/or treating PDAC in a subject that can include detecting one or more of the PDAC signatures.

### CROSS-REFERENCE TO RELATED APPLICATIONS

This application is a continuation of International Application No. PCT/US2023/063203, filed Feb. 24, 2023, which claims priority under 35 U.S.C. § 119 (e) of U.S. Provisional Patent Application No. 63/313,596, filed on Feb. 24, 2022, the contents of which is incorporated by reference herein in its entirety.

### SEQUENCE LISTING

This application contains a sequence listing filed in electronic form as an xml file entitled 114203-2256_ST26.xml, created on Aug. 22, 2024, and having a size of 21,407 bytes. The content of the sequence listing is incorporated herein in its entirety.

### TECHNICAL FIELD

The subject matter disclosed herein is generally directed to signatures, particularly gene expression signatures and tumor microenvironment immune signatures, of pancreatic cancer and uses thereof.

### BACKGROUND

Pancreatic ductal adenocarcinoma (PDAC) is projected to become the second leading cause of cancer death in the United States by 2030. Despite advancements in systemic therapy, many patients cannot receive post-operative chemotherapy and/or radiotherapy (CRT) due to the morbidity often associated with surgery. As such there exists a need for increased resolution of the cell landscape of PDAC and a corresponding development of improved treatments and preventions.

Citation or identification of any document in this application is not an admission that such a document is available as prior art to the present invention.

### SUMMARY

Described in certain example embodiments are methods of diagnosing, classifying and/or prognosing pancreatic ductal adenocarcinoma (PDAC), optionally time to progression (TTP) and/or overall survival (OS), in a subject in need thereof, comprising diagnosing the PDAC in the subject in need thereof, wherein diagnosing comprises detecting, in one or more PDAC tumor cells or organoids derived therefrom, (i) a malignant cell signature, program or both; (ii) a cancer-associated fibroblast (CAF) signature, program, or both; (iii) a tumor spatial community; (iv) one or more co-expressed receptor-ligand pairs; or (v) any combination thereof; wherein diagnosing, classifying and/or prognosing the PDAC is determined based on detection of one or more of the signatures, programs, communities, or receptor-ligand pairs.

Described in certain example embodiments herein are methods of treating pancreatic ductal adenocarcinoma (PDAC) in a subject in need thereof, comprising diagnosing, classifying, and/or prognosing the PDAC in the subject in need thereof, wherein diagnosing comprises detecting, in one or more PDAC tumor cells or organoids derived therefrom (i) a malignant cell signature, program or both; (ii) a cancer-associated fibroblast (CAF) signature, program, or both; (iii) a tumor spatial community; (iv) one or more co-expressed receptor-ligand pairs; or (v) any combination thereof; wherein diagnosing, classifying, and/or prognosing the PDAC is determined based on detection of one or more of the signatures, programs, communities, or receptor-ligand pairs; and administering, a PDAC treatment to the subject in need thereof, wherein the treatment optionally comprises a PDAC malignant cell modulating agent, a CAF modulating agent, an immune modulator, an apoptosis inhibitor, a TGFbeta modulator, a CXCR4 inhibitor, a HER2 inhibitor, or any combination thereof to the subject, wherein the PDAC treatment administered is based at least in part on the diagnosis, classification, and/or prognosis of the PDAC.

In certain example embodiments, the immune modulator is a myeloid cell agonist or antagonist.

In certain example embodiments, the PDAC malignant cell modulating agent and/or CAF modulating agent comprise a therapeutic antibody or fragment/combination thereof, antibody-like protein scaffold, aptamer, polypeptide, a polynucleotide, a genetic modifying agent or system, a small molecule therapeutic, a chemotherapeutic, small molecule degrader, inhibitor, an immunomodulator, or a combination thereof.

In certain example embodiments, the malignant cell signature or program comprises (i) a lineage specific expression program selected from a squamoid program, a mesenchymal program, a basaloid program, a classical-like program, an acinar-like program, a neuroendocrine-like program, a neural-like progenitor program, or any combination thereof; (ii) a cell state specific expression selected from a cycling(S) program, a cycling (G2/M) program, a TNF-NFkB signaling program, a MYC signaling program, an adhesive program, a ribosomal program, an interferon signaling program, or a combination thereof; (iii) a neoadjuvant treated malignant cell expression program; (iv) an untreated malignant cell expression program; or (v) any combination thereof.

In certain example embodiments, the neural-like progenitor program comprises one or more drug efflux programs and/or genes, apoptosis regulation programs and/or genes, chemoresistance programs and/or genes, tumor-nerve cross-talk programs and/or genes, neuronal gene expression programs, or neuronal development/migration/adhesion programs and/or genes, tissue stem cell module programs and/or genes, organ morphogenesis programs and/or genes, or hepatocyte nuclear factor activity programs and/or genes.

In certain example embodiments, the neural-like progenitor program comprises one or more genes selected from: CNTN4, CTNND2, NRXN3, RELN, SEMASA, NRCAM, AUTS2, ABCB1, BCL2, PDGFD, SPP1, SEMA3E, NFIB; any one or more genes in Table 5; any one or more genes in FIG. 15.

In certain example embodiments, the PDAC treatment inhibits or prevents, in one or more cells, expression of a malignant lineage program selected from a neural-like progenitor program, neuroendocrine-like program, basaloid program, mesenchymal program, or any combination thereof; an adhesive malignant state expression program; and/or a fibroblast adhesive program, or any combination thereof.

In certain example embodiments, the CAF signature or program comprises (i) a cell state specific expression program selected from an adhesive program, an immunomodulatory program, a myofibroblastic progenitor program, a neurotropic program, or a combination thereof; (ii) a neoadjuvant treated CAF expression program; (iii) an untreated CAF expression program; or (iv) any combination thereof.

In certain example embodiments, the PDAC treatment inhibits or prevents expression of a CAF adhesive program in one or more cells.

In certain example embodiments, the neoadjuvant treated malignant cell expression program comprises a malignant cell lineage program selected from a neural-like progenitor program, a neuroendocrine-like program, a mesenchymal program, a basaloid program, or a combination thereof; an adhesive malignant state expression program; or any combination thereof.

In certain example embodiments, the neoadjuvant treated CAF expression program comprises a fibroblast adhesive program.

In certain example embodiments, the tumor spatial community is a treatment-enriched community, a squamoid-basaloid community, or a classical community.

In certain example embodiments, the treatment-enriched community is enriched with cell(s) expressing a malignant cell lineage program selected from a neural-like progenitor program, a neuroendocrine-like program, a mesenchymal program, or an acinar-like program, or a combination thereof; cell(s) expressing a CAF expression program selected from a neurotropic program, an immunomodulatory program, or both; CD8+ T-cells; or any combination thereof.

In certain example embodiments, the squamoid-basaloid community is enriched with cell(s) expressing a malignant cell linage program selected from a squamoid program or a basaloid program, cells expressing a CAF immunomodulatory program, CD4+ T cells, B cells, regulatory T cells, natural killer cells, mast cells, conventional type 1 dendritic cells, plasmacytoid dendritic (pDC) cells, activated dendritic (aDC) cells, plasma cells.

In certain example embodiments, the classical community is enriched with cell(s) expressing a CAF myofibroblastic progenitor program, cell(s) expressing a CAF adhesive program, cell(s) expressing a malignant lineage classical-like program, macrophages, conventional type 2 dendritic cells, or any combination thereof.

In certain example embodiments, the tumor spatial community is enriched in cell(s) expressing a neuroendocrine-like program and/or a neural like malignant cell lineage program, CD8+ T cells, and conventional type 2 dendritic cells.

In certain example embodiments, the tumor spatial community is depleted of conventional type 1 dendritic cells.

In certain example embodiments, the one or more co-expressed receptor-ligand pairs are selected from FIG. 5B, FIG. 23, Table 3, or any combination thereof.

In certain example embodiments the method further comprises prognosing PDAC, optionally time to progression (TTP) and/or overall survival (OS), in the subject in need thereof, wherein prognosing is based at least in part on detection of one or more of the signatures, programs, communities, or receptor-ligand pairs.

In certain example embodiments, the TTP is predicted to be shorter for subjects with PDAC tumors or organoids derived therefrom expressing a malignant cell neural-like progenitor program and/or a squamoid program.

In certain example embodiments, the TTP is predicted to be longer for subjects with PDAC tumors or organoids derived therefrom expressing a malignant cell classical-like program and/or a CAF immunomodulatory program.

In certain example embodiments, the OS is predicted to be shorter for subjects with PDAC tumors or organoids derived therefrom expressing a malignant cell neural-like progenitor program and/or a squamoid program; and/or expressing a CAF adhesive program.

In certain example embodiments, the OS is predicted to be longer for subjects with PDAC tumors or organoids derived therefrom expressing a malignant cell classical-like program.

In certain example embodiments, the subject has had or is concurrently receiving a neoadjuvant therapy.

In certain example embodiments, detecting comprises a single cell RNA sequencing technique.

In certain example embodiments, detecting comprises a single-nucleus RNA sequencing technique.

In certain example embodiments, the single-nucleus RNA sequencing technique is optimized for pancreatic tissue.

In certain example embodiments, the single-nucleus RNA sequencing technique is optimized for frozen tissue.

In certain example embodiments, detecting comprises a spatially-resolved transcriptomics technique.

Described in certain example embodiments herein is a method of screening for one or more agents capable of treating or preventing PDAC or progression thereof comprising (a) contacting a PDAC tumor cell or cell population or an organoid or organoid cell population derived therefrom with a test agent or library of test agents, wherein the PDAC tumor cells or organoid cells have an initial cell state, expression signature, and/or expression program; (b) determining a fraction of PDAC or organoid cells having a desired cell state, expression signature, and/or expression program and/or determining a fraction of PDAC or organoid cells having an undesired cell state, expression signature, and/or expression program; and (c) selecting test agents that shift the initial PDAC or organoid cell state, expression signature, and/or expression program to a desired cell state, expression signature, and/or expression program and/or prevent a shift in the initial PDAC or organoid cell state, expression signature, and/or expression program to an undesired cell state, expression signature, and/or expression program or away from a desired cell state, expression signature, and/or expression program such that the fraction of PDAC and/or organoid cells having the desired cell state, expression signatures, and/or expression program is above a set cutoff limit.

In certain example embodiments, the desired PDAC or organoid cell state, expression signature, and/or expression program is a PDAC malignant cell classical-like program or a CAF immunomodulatory program.

In certain example embodiments, the undesired PDAC or organoid cell state, expression signature, and/or expression program is a PDAC malignant cell neural-like progenitor program, a PDAC malignant cell neuroendocrine-like program, a PDAC malignant cell squamoid program, a PDAC malignant cell basaloid program, a PDAC malignant cell mesenchymal program, or a CAF adhesive program.

In certain example embodiments, the initial cell state, expression signature, and/or expression program of the PDAC cell or cell population and/or the organoid or organoid cells is a PDAC malignant cell neural-like progenitor program.

In certain example embodiments, the PDAC tumor cell or cells are obtained from a subject in need thereof to be treated.

In certain example embodiments, the subject has had or is concurrently receiving a PDAC neoadjuvant therapy.

In certain example embodiments, the neural-like progenitor program comprises one or more drug efflux programs and/or genes, apoptosis regulation programs and/or genes, chemoresistance programs and/or genes, tumor-nerve cross-talk programs and/or genes, neuronal gene expression programs, or neuronal development/migration/adhesion programs and/or genes, tissue stem cell module programs and/or genes, organ morphogenesis programs and/or genes, or hepatocyte nuclear factor activity programs and/or genes.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1H—Single-nucleus RNA-seq of untreated and treated PDAC captures representative diversity of cell types including putative ADM intermediate. FIG. 1A, Experimental workflow of human PDAC tumors for snRNA-seq, Multiplex Ion Beam Imaging (MIBI), and digital spatial profiling (DSP; NanoString GeoMx). Three patient tumors were analyzed by DSP and not snRNA-seq and two specimens profiled by snRNA-seq were non-malignant pancreatic tissue. FIG. 1B, snRNA-seq captures diverse malignant, epithelial, immune and other stromal cell subsets. Mean normalized expression (greyscale bar) of selected marker genes (columns) across annotated cell subsets (rows) of different compartments (labels, left). FIG. 1C, Distinctions between patients or treatment status. UMAP embedding of single nucleus profiles (dots) of PDAC tumors colored by patient ID (color legend, left) or treatment status (right). FIG. 1D, Cell subsets in each compartment. UMAP embeddings of single nucleus profiles of all cells (left, as in FIG. 1C) or in each compartment (right panels) shaded by post hoc cell type annotations (greyscale legend). A small subset of acinar cells (acinar-REG) expressed high levels of regenerating family member genes (e.g., REGIA, REG3A), which have been implicated in promoting pancreatic inflammation, ADM and PanIN^{34,45,169,170}. FIG. 1E, snRNA-seq captures representative cell type distributions compared to in situ assessment. Left: Representative MIBI images and segmentation showing staining with antibodies against cytokeratin (green as represented in greyscale), vimentin (blue as represented in greyscale), CD45 (red as represented in greyscale), CD31 (purple as represented in greyscale) and double-stranded DNA (gray). Right: Proportion of cells (y axis) in each of the four major compartments (left panels, greyscale legend) or in each of the immune subsets (right panels, greyscale legend) as estimated by snRNA-seq or MIBI (x axis) in aggregate across all untreated (two left bars; n=5) or treated (two right bars; n=2) tumors. FIG. 1F, Remodeling of tumor composition by treatment. Proportions (y axis) of each cell subset (x-axis) among all nuclei. Pairwise comparisons were performed using the Mann-Whitney U test (* Bonferroni adjusted p<0.05; ** p<0.01; *** p<0.001). FIG. 1G-1H, Inferred differentiation states in pre-malignant and malignant cells. FIG. 1G, Proportion of cells (dot size) with non-zero expression of gene set HALLMARK_KRAS_SIGNALING_UP in each epithelial cell subset and normalized mean expression (dot shading) in expressing cells. FIG. 1H, Partition-based graph abstraction (PAGA) of an inferred pseudotemporal trajectory among epithelial cell subsets (nodes).

FIG. 2A-2D—Refined molecular taxonomy of PDAC identifies a novel neural-like progenitor program in malignant cells. FIG. 2A, Expression program dictionary in malignant cells and CAFs. UMAPs of single nucleus profiles (dots) of malignant cells (top and middle) and CAFs (bottom) from all tumors, shaded by patient (bottom right, malignant; bottom left, CAF) or by the normalized expression score of each program (see Methods in Working Examples). FIG. 2B, Distinctions between the neural-like progenitor and neuroendocrine-like programs. Overlap of each gene set (shaded pie charts) with the neural-like progenitor (green as represented in greyscale) and neuroendocrine-like (red as represented in greyscale) programs. Beige circles depict clusters of related gene sets. Edges represent overlaps between distinct gene sets based on an overlap coefficient threshold (>0.85, Cytoscape). FIG. 2C, The neural-like progenitor program includes ‘brain tissue enhanced’ genes from the Human Protein Atlas (HPA). Left: Overlap between the program (blue as represented in greyscale) and HPA brain enhanced (orange as represented in greyscale) genes. Right: HPA expression categories (greyscale code) for select genes (columns) across brain regions (rows). FIG. 2D, Multiplexed immunofluorescence images of independent PDAC specimen showing absence of NRXN3 expression (top) and heterogeneous NRXN3 expression (bottom) in malignant cells/glands from two separate regions of the same tumor. Greyscale legend indicates target of fluorophore-conjugated antibodies.

FIG. 3A-3E—The neural-like progenitor program is enriched in residual tumor and patient-derived organoids after cytotoxic therapy and is associated with poor clinical outcomes. FIG. 3A, Intra-tumoral and inter-tumoral heterogeneity of malignant and fibroblast expression programs. Normalized expression scores (y axis) of malignant state (top), malignant lineage (middle) and CAF (bottom) programs (greyscale legend) in each untreated (n=18, left) or treated (n=25, right) tumor (x axis). Treated patients are further ordered by treatment regimen. FIG. 3B, Malignant cell and CAF programs associated with treatment status. Mean normalized program expression (y axis) of malignant cell state (top), malignant cell lineage (middle), and CAF (bottom) programs (x axis) in untreated (n=18), CRT (n=14), and CRTL (n=5) tumors. * Bonferroni adjusted p<0.05, ** p<0.01, *** p<0.001, **** p<0.0001, Mann-Whitney U test. FIG. 3C, Expression of malignant lineage programs in residual neoplastic cells varies by patients' treatment response. Distribution of mean normalized expression scores in each tumor (y axis) for each pathological treatment response grade (grayscale legend) for each malignant lineage program (x axis) regardless of treatment group. * Bonferroni adjusted p<0.05, ** p<0.01, *** p<0.001, **** p<0.0001, Mann-Whitney U test. FIG. 3D, The neural-like progenitor program increases in organoids following CRT treatment. Distribution of mean expression of the top 200 cNMF-weighted genes from the neural-like progenitor program (y axis) across individual cells from matched untreated and CRT-treated organoids (x axis) derived from patient PDAC_U_12 (p=1.33×10⁻¹⁵; Mann-Whitney U test). FIG. 3E, Program expression and clinicopathologic parameters associated with time to disease progression using multivariable Cox regression analysis of bulk RNA-seq data from two independent cohorts of untreated, resected primary PDAC (TCGA and PanCuRx; n=266). Malignant lineage: NRP=neural-like progenitor, SQM=squamoid, MES =mesenchymal, ACN=acinar-like, NEN=neuroendocrine-like, BSL=basaloid, CLS=classical. Malignant state (aggregate): CYC=cycling, SEC=secretory. Fibroblast: IMM=immunomodulatory, NRT=neurotropic, ADH-F=adhesive, MYO=myofibroblastic progenitor.

FIG. 4A-4D—Spatial mapping of malignant programs, CAF programs and immune cell composition in untreated and treated PDAC tumors reveals three distinct multicellular communities. FIG. 4A, Whole Transcriptome Digital Spatial Profiling (WTA DSP). Left: Representative hematoxylin and eosin (H&E)-stained FFPE sections (5 μm thickness, left) and immunofluorescence image (GeoMx DSP, right) of consecutive sections from the same tumor FFPE block, showing selected regions of interest (ROIs, circles). Gray=SYTO13 (nuclear stain), green—as represented in greyscale=anti-panCK, magenta—as represented in greyscale=anti-CD45, cyan—as represented in greyscale=anti-αSMA. Right: Example ROI (circle, 600 μm diameter) with segmentation masks used to enrich for the epithelial, CAF, and immune compartments and percent of total segment area occupied by each compartment. FIG. 4B, Higher variation across tumors than within tumor ROIs. Left: Normalized expression (shading scale) of malignant cell (top rows) and fibroblast (bottom rows) programs in each AOI (columns) across patients (greyscale bar 2, shading legend) and treatment status (greyscale bar 1 and greyscale legend). Right: Program expression variation between patients (y axis, interquartile range/IQR of the mean program score for each tumor) and within patients (x axis, mean of IQR across all ROIs within a tumor). Dotted line x=y. FIG. 4C-4D, Three multicellular communities with distinct malignant, CAF, and immune features. FIG. 4C, Pearson correlation coefficient (greyscale bar) of the scores/proportions of each malignant, CAF, and immune feature (rows, columns) across ROIs. Rows and columns are ordered by hierarchical clustering. FIG. 4D, Schematic of key features of each multicellular community as defined in FIG. 4C. Malignant lineage: NRP=neural-like progenitor, SQM=squamoid, MES=mesenchymal, ACN=acinar-like, NEN=neuroendocrine-like, BSL=basaloid, CLS=classical. Fibroblast: IMM=immunomodulatory, NRT=neurotropic, ADH-F=adhesive, MYO=myofibroblastic progenitor.

FIG. 5A-5C—Spatially-defined associations of malignant programs and intercellular receptor-ligand interactions as a function of treatment. FIG. 5A, Malignant and CAF programs associated with immune cell composition. Fold change (greyscale bar) of inferred immune subset proportions (rows) between the top quartile scoring ROIs and the bottom quartile scoring ROIs for each malignant (columns; left) or fibroblast (columns; right) program. FIG. 5B, Spatially correlated receptor-ligand pairs across compartments. Spearman rank correlation coefficient of expression of receptor-ligand pairs (gray dots) across paired epithelial: CAF (left), epithelial: immune (middle), or CAF: immune (right) segments within the same ROI across all ROIs in CRT-treated (y axis) or untreated (x axis) tumors. Selected receptor-ligand pairs that were differentially correlated in CRT-treated or untreated tumors are labeled and shaded based on the segment expressing the ligand (greyscale legend). Dotted line: x=y. FIG. 5C, Cell intrinsic, clinical, and spatial associations for malignant lineage programs (columns). Malignant lineage: NRP=neural-like progenitor, SQM=squamoid, MES=mesenchymal, ACN=acinar-like, NEN=neuroendocrine-like, BSL=basaloid, CLS=classical. Malignant state: CYS=cycling(S), CYG =cycling (G2/M), MYC=MYC signaling, ADH-M=adhesive, RIB=ribosomal, IFN=interferon signaling, TNF=TNF-NFκB signaling. Fibroblast: IMM=immunomodulatory, NRT=neurotropic, ADH-F=adhesive, MYO=myofibroblastic progenitor.

FIG. 6A-6B—Cell type composition across PDAC tumors. FIG. 6A, UMAP embeddings of single nucleus profiles (dots) from individual tumors (panels) from untreated (left) and treated (right) patients shaded by post hoc cell type annotations (greyscale legend). FIG. 6B, Cell type distributions across tumors. Proportions (y axis) of cell subsets (greyscale legend) across untreated (n=18) (left) vs. treated (n=25) tumors (right) either with (top) or without (bottom) malignant cells. Treated patients are further classified by specific treatment type.

FIG. 7A-7B—Inferred CNAs recapitulate prior PDAC genomic studies. FIG. 7A, Example inferCNV analysis of the epithelial subset from a study specimen. Inferred amplifications (darker greys) and deletions (lighter greys) based on expression (greyscalebar) of sliding 100-gene window in each chromosomal locus (columns) from each cell (rows) labeled by its annotated cell type (shaded code). FIG. 7B, Inferred CNA frequencies in the snRNA-seq cohort have similar distribution as those derived from TCGA genomic studyl1. Frequency (y axis) of CNAs on each chromosome arm (x axis) as inferred across the patients in the snRNA-seq cohort (light grey bars) and from genome analysis of PDAC (dark grey bars) from the TCGA cohort.

FIG. 8—snRNA-seq captures representative cell type distributions compared to in situ assessment by MIBI. Proportion of cells (y axis) in each of the four major compartments (greyscale legend, top) or immune cell subsets (greyscale legend, bottom) as estimated by snRNA-seq or MIBI (x axis) in each matched untreated (left; n=5) or treated (right; n=2) tumor.

FIG. 9—Treatment associated with distinct cell type proportions across compartments. Proportions (y axis) of cell types (x axis) in untreated (n=18), CRT (n=14), or CRTL (n=5) tumors out of all non-malignant cells (top left) or in specific non-malignant cell compartments in the tumor. * Bonferroni adjusted p<0.05, ** p<0.01, *** p<0.001, Mann-Whitney U test.

FIG. 10—Impact of treatment on differential gene expression in immune cells. Differential expression (β-value, x axis, mixed-effects model) and its significance (−log₁₀ (adjusted p-value), y axis) for CD8⁺ T cells (top row), dendritic cells (second row), T_{regs} (third row) and macrophages (bottom row, greyscale legend) in CRT vs. untreated (left), CRTL vs. untreated (middle), and CRTL vs. CRT (right) tumors. Selected enriched or depleted genes are labeled. Bonferroni adjusted p-value <0.05 is indicated with a dotted horizontal line.

FIG. 11—Impact of treatment on differential gene expression in malignant cells and fibroblasts. Differential expression (β-value, x axis, mixed-effects model) and its significance (−log₁₀(adjusted p-value), y axis) for malignant cells (top row) and CAFs (bottom row, greyscale legend) in CRT vs. untreated (left), CRTL vs. untreated (middle), and CRTL vs. CRT (right) tumors. Selected enriched or depleted genes are labeled. Bonferroni adjusted p-value <0.05 is indicated with a dotted horizontal line.

FIG. 12A-12C—Epithelial cell type composition across PDAC tumors. FIG. 12A, UMAP embeddings of single nucleus profiles (dots) for different epithelial cell subsets (panels) shaded by patient ID (greyscale legend). FIG. 12B, Left: proportions (y axis) of cells in each tumor (greyscale legend) for each epithelial cell subset (x axis); Right: proportions (y axis) of epithelial cell subsets (greyscale legend) for each tumor (x axis). FIG. 12C, Proportions (y axis) of epithelial cell subsets (greyscale legend) summed across all tumors for each treatment category (x axis).

FIG. 13A-13B-Prior signatures derived primarily from the bulk setting insufficiently delineate cells from snRNA-seq. FIG. 13A, Malignant cell signatures. UMAP embeddings of single nucleus profiles (dots) from all tumor nuclei (top panels) or only malignant cells (bottom panels) shaded by expression score (greyscale bar, Methods) of signatures derived from the Baileyl¹⁰, Collisson⁶, Moffitt⁹, and Chan-Seng-Yue⁶⁹ studies. FIG. 13B, CAF signatures. UMAP embeddings of single nucleus profiles (dots) from all fibroblast nuclei colored by normalized expression score (shaded bar, Methods) of myCAF, apCAF, and iCAF signatures49 and well as cross-tissue fibroblast lineage signatures (COL3A1⁺ myofibroblast, LRRC15⁺ myofibroblast, CCL19⁺ colitis, ADAMDECT colitis, NPNT alveolar, and PI16⁺ adventitial) 72.

FIG. 14A-14B—Stability and power in selection of programs in consensus NMF. FIG. 14A, Estimated stability (black, left y axis) and error (grey, right y axis) in the cNMF solution learned with different numbers of programs (k, x axis) for malignant cells (left) and CAFs (right). FIG. 14B, Number of malignant (out of 14; left) and CAF (out of 4; right) programs recovered in the cNMF solution learned with a different proportion of samples (x axis) subsampled from our cohort.

FIG. 15—Overlap between the neural-like progenitor program signature and genes upregulated in association with perineural invasion in PDAC. Differential expression (log₂(fold-change), x axis) and its significance (—log₁₀(adjusted p-value), y axis, DESeq2) of TCGA PDAC patients with (right) and without (left) perineural invasion (PNI). Labeled genes are present in the neural-like progenitor program signature.

FIG. 16A-16B—Correlation among malignant cell or CAF expression programs. Correlation (color bar) among expression scores of malignant state and lineage programs across all malignant nuclei (FIG. 16A) or fibroblast programs across all fibroblast nuclei (FIG. 16B).

FIG. 17—Enrichment of malignant cell and CAF programs in genes differentially expressed with treatment regimen. Fold enrichment of overlap (x axis) between gene program signatures (top 200 genes; rows) and genes differentially expressed (q<0.05) in CRT vs. untreated (left), CRTL vs. untreated (middle), or CRTL vs. CRT (right). * Bonferroni adjusted p<0.05, hypergeometric test.

FIG. 18—Multivariable Cox regression analysis for overall survival in TCGA and PanCuRx PDAC cohorts. Hazard ratios (middle) and p-values (left) for each variable (clinicopathologic and program expression score in bulk RNA-seq, rows) in multivariable Cox regression model for overall survival (OS), based on a cohort of 266 patients with untreated, resected primary PDAC profiled by RNA-seq in TCGA and PanCuRx.

FIG. 19—Digital Spatial Profiling (DSP) with whole transcriptome assay (WTA). Immunofluorescence images of FFPE sections from all PDAC specimens analyzed using whole transcriptome DSP separated by treatment status (top, untreated; bottom, treated). Greyscale legend indicates target of fluorophore-conjugated antibodies.

FIG. 20—Digital spatial profiling with whole transcriptome atlas enables accurate mapping of cell type signatures in space. Expression (z-score of normalized counts across segments; shaded bar) of signature genes (rows) from different cell types (greyscale legend 3 and left greyscale bar 3) across segments (columns, greyscale legend 2 and horizontal greyscale bar 2) and treatment regimens (columns, grayscale legend 1 and horizontal grayscale bar 1) profiled by WTA, capturing epithelial (green, as represented in greyscale), fibroblasts (blue, as represented in greyscale) and immune (red, as represented in greyscale) cells. Columns and rows are clustered by unsupervised hierarchical clustering.

FIG. 21A-21B—Digital spatial profiling shows enrichment of neural-like progenitor and neuroendocrine-like program after neoadjuvant CRT. Distribution of z-score normalized ssGSEA enrichment scores (y axis) of malignant (FIG. 21A) and fibroblast (FIG. 21B) programs (x axis) in AOIs from CRT (gray) and untreated (white) tumors. Box depicts interquartile range (IQR) with median marked as horizontal line. The whiskers correspond to 1.5 x IQR. * p<0.05, mixed-effects model.

FIG. 22—Association of malignant, CAF, and immune features across tumors based on snRNA-seq. Pearson correlation coefficient (color bar) of the scores of each CAF, malignant, and immune feature in snRNA-seq (rows, columns) across patient tumors. Rows and columns are ordered by hierarchical clustering.

FIG. 23—Spatially correlated receptor-ligand pairs within compartments. Spearman rank correlation coefficient of expression of receptor-ligand pairs (gray dots) within the epithelial (left), CAF (middle) or immune (right) segments in the same ROI across all ROIs in CRT-treated (y axis) or untreated (x axis) tumors. Select receptor-ligand pairs that were differentially correlated in CRT-treated or untreated tumors are labeled. Solid line: x=y.

FIG. 24—snRNA-seq captures a greater diversity and abundance of cell types relative to prior single-cell approaches. Number of nuclei/cells per untreated tumor that passed quality control filters (y axis) in our study (n=18) vs. Peng et al. study (n=24)⁵¹ (grayscale legend), in total (left) and partitioned by cell type (right). * p<0.05, ** p<0.01, *** p<0.001, **** p<0.0001, Mann-Whitney U test.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to U.S. Provisional Application Ser. No. 63/069,035 and PCT Application Serial Number PCT/2021/047041.

All publications, published patent documents, and patent applications cited herein are hereby incorporated by reference to the same extent as though each individual publication, published patent document, or patent application was specifically and individually indicated as being incorporated by reference.

### OVERVIEW

Pancreatic ductal adenocarcinoma (PDAC) is projected to become the second leading cause of cancer death in the United States by 2030. Pancreatic ductal adenocarcinoma (PDAC) remains a treatment-refractory disease. Characterizing PDAC by mRNA profiling remains particularly challenging. Previously identified bulk expression subtypes were influenced by contaminating stroma and have not yet translated into meaningful information for clinical management. Single cell RNA-seq (scRNA-seq) of fresh tumors under-represented key cell types and also thus failed to translate into clinically relevant information. More specifically, although single cell RNA-seq (scRNA-seq) can resolve these questions by distinguishing the diversity of malignant and non-malignant cells in the tumor and elucidating the impact of therapy on each compartment and their interactions, scRNA-seq in PDAC has lagged behind other cancer types due to high intrinsic nuclease content and dense desmoplastic stroma, resulting in reduced RNA quality, low numbers of viable cells, preferential capture of certain cell types at the expense of others, and challenges with dissociating treated tumors.

Described in exemplary embodiments herein are robust single-nucleus RNA-seq (snRNA-seq) and spatial transcriptomics techniques optimized for frozen archival samples which are demonstrated using PDAC specimens. As is demonstrated in e.g., the Working Examples herein, PDAC samples from untreated and those that were from subjects that received neoadjuvant chemotherapy and radiotherapy (CRT) were analyzed using these techniques, which resulted in gene expression programs and signatures for previously unresolved subtypes and of PDAC cells. Embodiments disclosed herein provide expression signatures of PDAC tumors and methods of their use in a clinically relevant context to, among other things, improve patient treatment and prognostic stratification.

Described in certain example embodiments are methods of diagnosing, classifying and/or prognosing pancreatic ductal adenocarcinoma (PDAC), optionally time to progression (TTP) and/or overall survival (OS), in a subject in need thereof, comprising diagnosing the PDAC in the subject in need thereof, wherein diagnosing comprises detecting, in one or more PDAC tumor cells or organoids derived therefrom, (i) a malignant cell signature, program or both; (ii) a cancer-associated fibroblast (CAF) signature, program, or both; (iii) a tumor spatial community; (iv) one or more co-expressed receptor-ligand pairs; or (v) any combination thereof; wherein diagnosing, classifying and/or prognosing the PDAC is determined based on detection of one or more of the signatures, programs, communities, or receptor-ligand pairs.

Described in certain example embodiments herein are methods of treating pancreatic ductal adenocarcinoma (PDAC) in a subject in need thereof, comprising diagnosing, classifying, and/or prognosing the PDAC in the subject in need thereof, wherein diagnosing comprises detecting, in one or more PDAC tumor cells or organoids derived therefrom (i) a malignant cell signature, program or both; (ii) a cancer-associated fibroblast (CAF) signature, program, or both; (iii) a tumor spatial community; (iv) one or more co-expressed receptor-ligand pairs; or (v) any combination thereof; wherein diagnosing, classifying, and/or prognosing the PDAC is determined based on detection of one or more of the signatures, programs, communities, or receptor-ligand pairs; and administering, a PDAC treatment to the subject in need thereof, wherein the treatment optionally comprises a PDAC malignant cell modulating agent, a CAF modulating agent, an immune modulator, an apoptosis inhibitor, a TGFbeta modulator, a CXCR4 inhibitor, a HER2 inhibitor, or any combination thereof to the subject, wherein the PDAC treatment administered is based at least in part on the diagnosis, classification, and/or prognosis of the PDAC.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Expression Signatures and Programs

Described herein are PDAC tumor signatures and/or programs including, but not limited to, a malignant signature and/or program, a CAF signature and/or program, a tumor spatial community; one or more co-expressed receptor-ligand pairs or any combination thereof. In some embodiments the PDAC tumor signatures and/or programs include a neoadjuvant treated tumor expression program (“a treated program”); or a neoadjuvant untreated tumor expression program (an “untreated program”). In some embodiments, the PDAC tumor signature and/or program is a malignant cell signature and/or program. In some embodiments, the PDAC tumor signature and/or program is a CAF signature and/or program. Such expression signatures and/or programs can be used, for example, in a method of diagnosing, classifying, prognosing, and/or the like PDAC in a subject. These and other exemplary methods are described in greater detail elsewhere herein.

In certain example embodiments, the therapeutic, diagnostic, and screening methods disclosed herein target, detect, or otherwise make use of one or more biomarkers of an expression signature. As used herein, the term “biomarker” can refer to a gene, an mRNA, cDNA, an antisense transcript, a miRNA, a polypeptide, a protein, a protein fragment, or any other nucleic acid sequence or polypeptide sequence that indicates either gene expression levels or protein production levels. Accordingly, it should be understood that reference to a “signature” in the context of those embodiments may encompass any biomarker or biomarkers whose expression profile or whose occurrence is associated with a specific cell type, subtype, or cell state of a specific cell type or subtype within a population of cells (e.g., Synovial Sarcoma cells) or a specific biological program. As used herein the term “module” or “biological program” can be used interchangeably with “expression program” and refers to a set of biomarkers that share a role in a biological function (e.g., an activation program, cell differentiation program, proliferation program). Biological programs can include a pattern of biomarker expression that result in a corresponding physiological event or phenotypic trait. Biological programs can include up to several hundred biomarkers that are expressed in a spatially and temporally controlled fashion. Expression of individual biomarkers can be shared between biological programs. Expression of individual biomarkers can be shared among different single cell types; however, expression of a biological program may be cell type specific or temporally specific (e.g., the biological program is expressed in a cell type at a specific time). Expression of a biological program may be regulated by a master switch, such as a nuclear receptor or transcription factor. As used herein, the term “topic” refers to a biological program. Topics are described further herein. The biological program (topic) can be modeled as a distribution over expressed biomarkers.

In certain embodiments, the expression of the signatures disclosed herein (e.g., core oncogenic signature) is dependent on epigenetic modification of the biomarkers or regulatory elements associated with the signatures (e.g., chromatin modifications or chromatin accessibility). Thus, in certain embodiments, use of signature biomarkers includes epigenetic modifications of the biomarkers that may be detected or modulated. As used herein, the terms “signature”, “expression profile”, or “expression program” may be used interchangeably (e.g., expression of genes, expression of gene products or polypeptides). It is to be understood that also when referring to proteins (e.g., differentially expressed proteins), such may fall within the definition of “gene” signature. Levels of expression or activity may be compared between different cells in order to characterize or identify for instance signatures specific for cell (sub) populations. Increased or decreased expression or activity or prevalence of signature biomarkers may be compared between different cells in order to characterize or identify for instance specific cell (sub) populations. The detection of a signature in single cells may be used to identify and quantitate, for instance, specific cell (sub) populations. A signature may include a biomarker whose expression or occurrence is specific to a cell (sub) population, such that expression or occurrence is exclusive to the cell (sub) population. An expression signature as used herein, may thus refer to any set of up- and/or down-regulated biomarkers that are representative of a cell type or subtype. An expression signature as used herein, may also refer to any set of up- and/or down-regulated biomarkers between different cells or cell (sub) populations derived from a gene-expression profile. For example, an expression signature may comprise a list of biomarkers differentially expressed in a distinction of interest. A signature can also include a cell type and/or cell state distribution. The cell type distribution can, for example, be indicative of the state of a population of cells or tissue, such as a tumor tissue, and/or a microenvironment of a tissue or population of cells, and/or a niche microenvironment within a tissue or cell population.

The signature according to certain embodiments of the present invention may comprise or consist of one or more biomarkers, such as for instance 1, 2, 3, 4, 5, 6, 7, 8, 9, 10 or more. In certain embodiments, the signature may comprise or consist of two or more biomarkers, such as for instance 2, 3, 4, 5, 6, 7, 8, 9, 10 or more. In certain embodiments, the signature may comprise or consist of three or more biomarkers, such as for instance 3, 4, 5, 6, 7, 8, 9, 10 or more. In certain embodiments, the signature may comprise or consist of four or more biomarkers, such as for instance 4, 5, 6, 7, 8, 9, 10 or more. In certain embodiments, the signature may comprise or consist of five or more biomarkers, such as for instance 5, 6, 7, 8, 9, 10 or more. In certain embodiments, the signature may comprise or consist of six or more biomarkers for instance 6, 7, 8, 9, 10 or more. In certain embodiments, the signature may comprise or consist of seven or more biomarkers, such as for instance 7, 8, 9, 10 or more. In certain embodiments, the signature may comprise or consist of eight or more biomarkers, such as for instance 8, 9, 10 or more. In certain embodiments, the signature may comprise or consist of nine or more biomarkers, such as for instance 9, 10 or more. In certain embodiments, the signature may comprise or consist of ten or more biomarkers, such as for instance 10, 11, 12, 13, 14, 15, or more. It is to be understood that a signature according to the invention may for instance also include different types of biomarkers combined (e.g., genes and proteins).

In certain embodiments, a signature is characterized as being specific for a particular cell or cell (sub) population if it is upregulated or only present, detected or detectable in that particular cell or cell (sub) population, or alternatively is downregulated or only absent, or undetectable in that particular cell or cell (sub) population. In this context, a signature consists of one or more differentially expressed genes/proteins or differential epigenetic elements when comparing different cells or cell (sub) populations, including comparing different cells or cell (sub) populations (e.g., synovial sarcoma cells), as well as comparing malignant cells or malignant cell (sub) populations with other non-malignant cells or non-malignant cell (sub) populations. It is to be understood that “differentially expressed” biomarkers include biomarkers which are up- or down-regulated as well as biomarkers which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art. Differential expression of biomarkers may also be determined by comparing expression of biomarkers in a population of cells or in a single cell. In certain embodiments, expression of one or more biomarkers is mutually exclusive in cells having a different cell state or subtype (e.g., two genes are not expressed at the same time). In certain embodiments, a specific signature may have one or more biomarkers upregulated or downregulated as compared to other biomarkers in the signature within a single cell. In certain embodiments, a specific signature may have one or more biomarkers upregulated or downregulated as compared to other biomarkers in the signature within a single nucleus within a cell. Thus, a cell type or subtype can be determined by determining the pattern of expression in a single cell and/or a single nucleus within a cell.

As discussed herein, differentially expressed biomarkers may be differentially expressed on a single cell level or may be differentially expressed on a cell population level. Preferably, the differentially expressed biomarkers as discussed herein, such as constituting the expression signatures as discussed herein, when as to the cell population level, refer to biomarkers that are differentially expressed in all or substantially all cells of the population (such as at least 80%, preferably at least 90%, such as at least 95% of the individual cells). This allows one to define a particular subpopulation of cells. As referred to herein, a “subpopulation” of cells preferably refers to a particular subset of cells of a particular cell type (e.g., Synovial Sarcoma) which can be distinguished or are uniquely identifiable and set apart from other cells of this cell type. The cell subpopulation may be phenotypically characterized and is preferably characterized by the signature as discussed herein. A cell (sub) population as referred to herein may constitute of a (sub) population of cells of a particular cell type characterized by a specific cell state.

When referring to induction, or alternatively suppression of a particular signature, preferable is meant induction or alternatively suppression (or upregulation or downregulation) of at least one biomarker of the signature, such as for instance at least two, at least three, at least four, at least five, at least six, or all biomarkers of the signature.

Example gene signatures and topics are further described below.

### Malignant Signatures and Programs

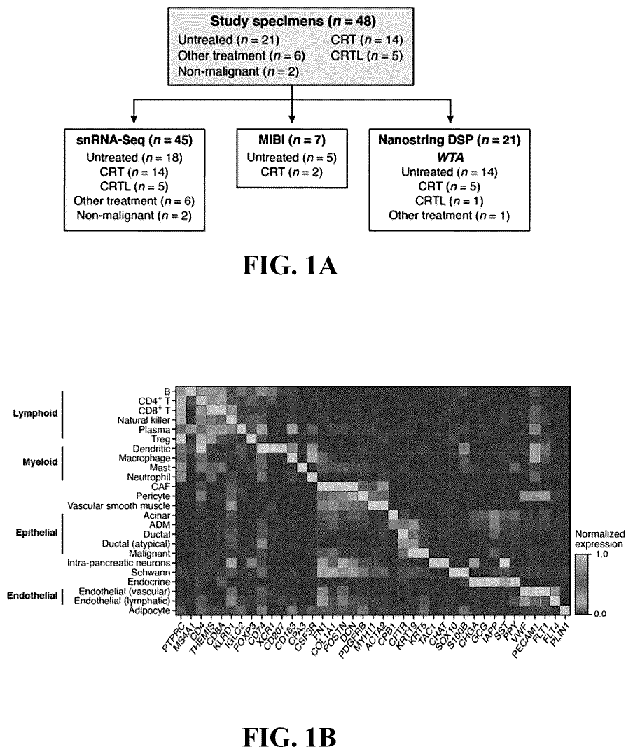

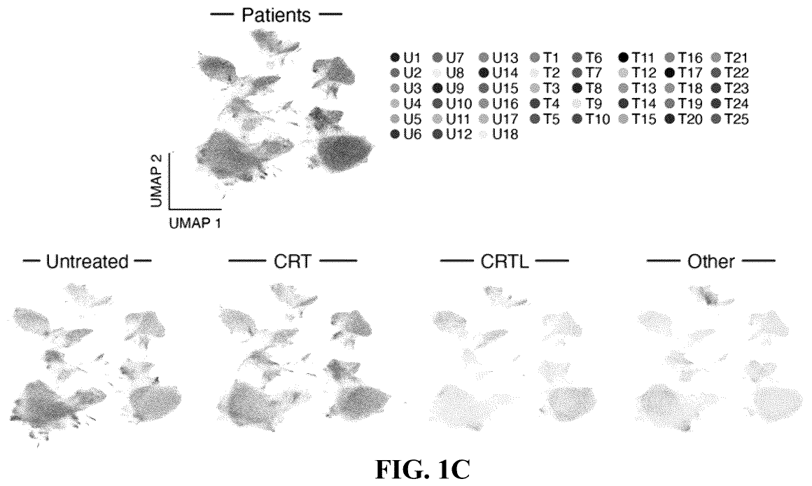

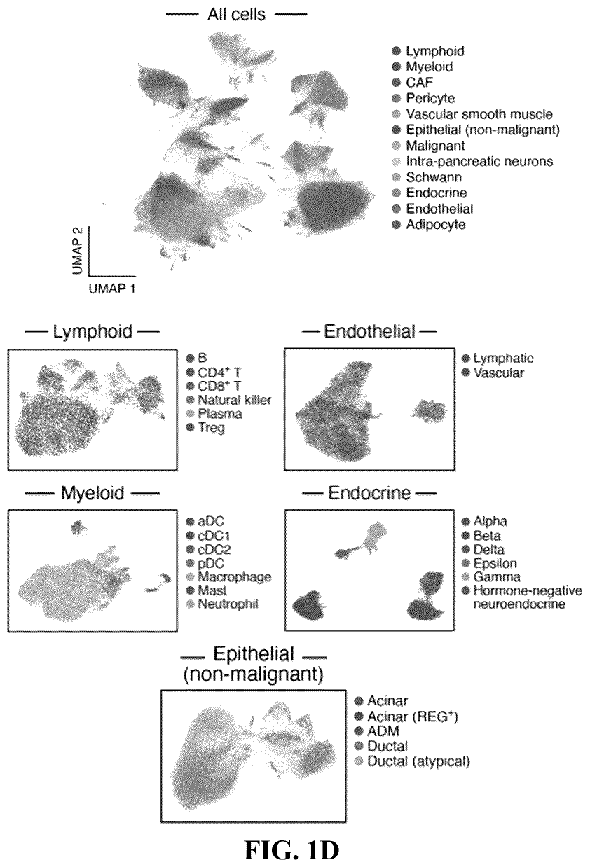

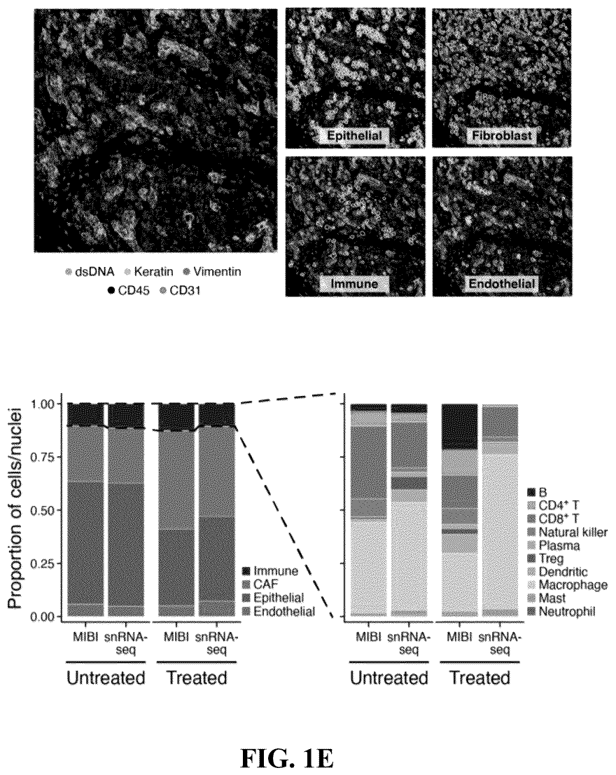

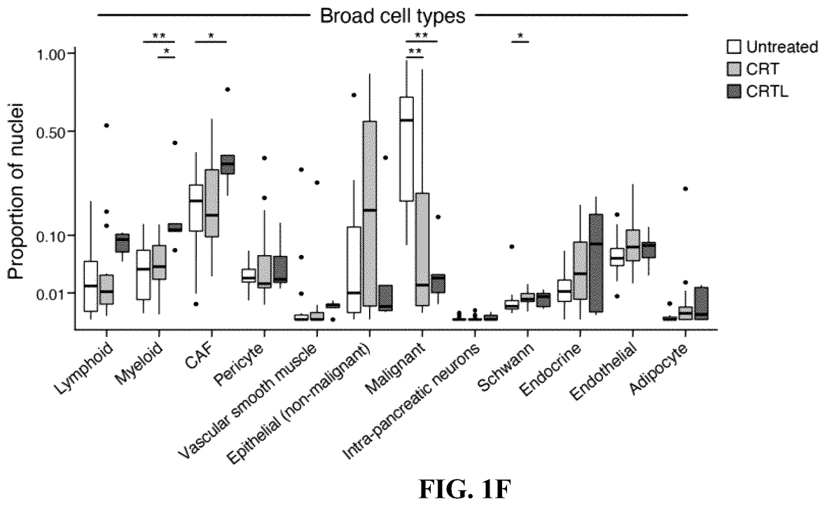

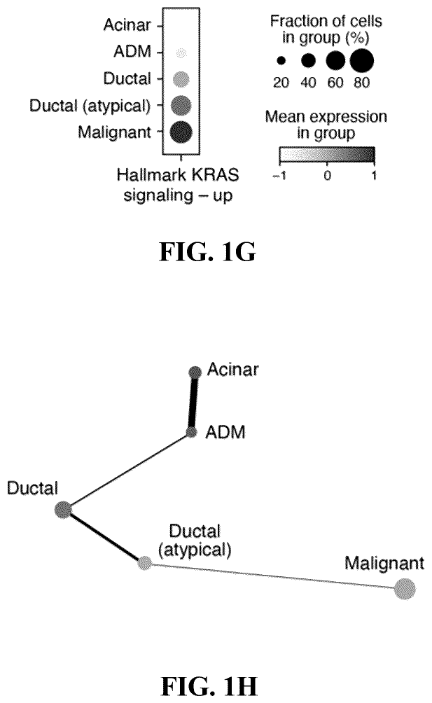

In some embodiments the PDAC tumor signature and/or program is or includes a malignant signature and/or program. In some embodiments, the malignant signature and/or program is or includes of a neoadjuvant treated signature and/or program. In certain embodiments, a malignant signature (e.g., signature of differentially expressed genes between malignant cells and non-malignant cells, e.g., epithelial cells, CAFs, CD8 and CD4 T cells, B cells, NK cells, macrophages, or mastocytes) comprises one or more biomarkers, expression programs, biologic programs, receptor-ligand interactions, cell state distribution, cell type distribution, or any combination thereof as in any of FIGS. 1B-1G, 2A-2D, 3A-3E, 4B-4D, 5A-5C, 6-13, 15-24, Tables 2, 3, 4, 5, 6, 7A-7B, or any combination thereof.

In certain example embodiments, the malignant cell signature or program comprises (i) a lineage specific expression program selected from a squamoid program, a mesenchymal program, a basaloid program, a classical-like program, an acinar-like program, a neuroendocrine-like program, a neural-like progenitor program, or any combination thereof; (ii) a cell state specific expression selected from a cycling(S) program, a cycling (G2/M) program, a TNF-NFkB signaling program, a MYC signaling program, an adhesive program, a ribosomal program, an interferon signaling program, or a combination thereof; (iii) a neoadjuvant treated malignant cell expression program; (iv) an untreated malignant cell expression program; or (v) any combination thereof.

In certain example embodiments, the neural-like progenitor program comprises one or more drug efflux programs and/or genes, apoptosis regulation programs and/or genes, chemoresistance programs and/or genes, tumor-nerve cross-talk programs and/or genes, neuronal gene expression programs, or neuronal development/migration/adhesion programs and/or genes, tissue stem cell module programs and/or genes, organ morphogenesis programs and/or genes, or hepatocyte nuclear factor activity programs and/or genes.

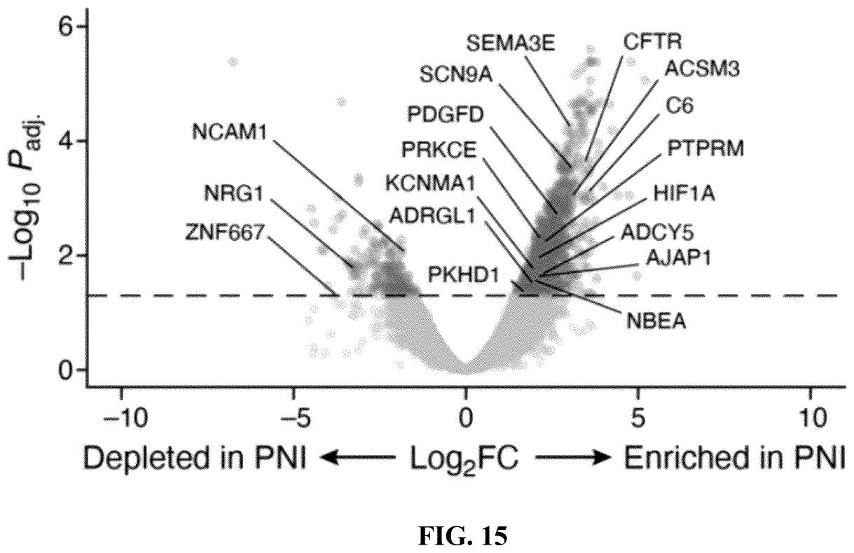

In certain example embodiments, the neural-like progenitor program comprises one or more genes selected from: CNTN4, CTNND2, NRXN3, RELN, SEMASA, NRCAM, AUTS2, ABCB1, BCL2, PDGFD, SPP1, SEMA3E, NFIB; any one or more genes in Table 5; any one or more genes in FIG. 15.

### Malignant Neoadjuvant Treated and Untreated Programs

In some embodiments, the malignant signature and/or program is or includes a neoadjuvant treated malignant signature and/or program (i.e., a signature specific to malignant cells that have undergone a neoadjuvant treatment). In some embodiments, the malignant signature and/or program is or includes a neoadjuvant untreated malignant signature (i.e., a signature specific to malignant cells that have not undergone a neoadjuvant treatment).

In certain example embodiments, the neoadjuvant treated malignant cell expression program comprises a malignant cell lineage program selected from a neural-like progenitor program, a neuroendocrine-like program, a mesenchymal program, a basaloid program, or a combination thereof; an adhesive malignant state expression program; or any combination thereof.

In some embodiments, the neoadjuvant treated malignant signature and/or program comprises one or more biomarkers, expression programs, biologic programs, receptor-ligand interactions, cell state distribution, cell type distribution, or any combination thereof as in any of FIGS. 1B-1G, 2A-2D, 3A-3E, 4B-4D, 5A-5C, 6-13, 15-24, Tables 2, 3, 4, 5, 6, 7A-7B, or any combination thereof.

### Cancer Associated Fibroblast (CAF) Signatures and Programs

In some embodiments the PDAC tumor signature is or includes a CAF signature and/or program. In some embodiments, the CAF signature and/or program comprises one or more biomarkers, expression programs, biologic programs, receptor-ligand interactions, cell state distribution, cell type distribution, or any combination thereof as in any of FIGS. 1B-1G, 2A-2D, 3A-3E, 4B-4D, 5A-5C, 6-13, 15-24, Tables 2, 3, 4, 5, 6, 7A-7B, or any combination thereof.

In certain example embodiments, the CAF signature or program comprises (i) a cell state specific expression program selected from an adhesive program, an immunomodulatory program, a myofibroblastic progenitor program, a neurotropic program, or a combination thereof; (ii) a neoadjuvant treated CAF expression program; (iii) an untreated CAF expression program; or (iv) any combination thereof.

### CAF Neoadjuvant Treated and Untreated Signatures and Programs

In some embodiments, the CAF signature and/or program is or includes a neoadjuvant treated CAF signature and/or program (i.e., a signature specific and/or program to CAFs that have undergone a neoadjuvant treatment). In some embodiments, the malignant signature is or includes a neoadjuvant untreated malignant signature (i.e., a signature specific and/or program to CAFs that have not undergone a neoadjuvant treatment).

In certain example embodiments, the neoadjuvant treated CAF expression program comprises a fibroblast adhesive program.

In some embodiments, the neoadjuvant treated CAF expression signature and/or program comprises one or more biomarkers, expression programs, biologic programs, receptor-ligand interactions, cell state distribution, cell type distribution, or any combination thereof as in any of FIGS. FIGS. 1B-1G, 2A-2D, 3A-3E, 4B-4D, 5A-5C, 6-13, 15-24, Tables 2, 3, 4, 5, 6, 7A-7B, or any combination thereof.

### Tumor Spatial Communities

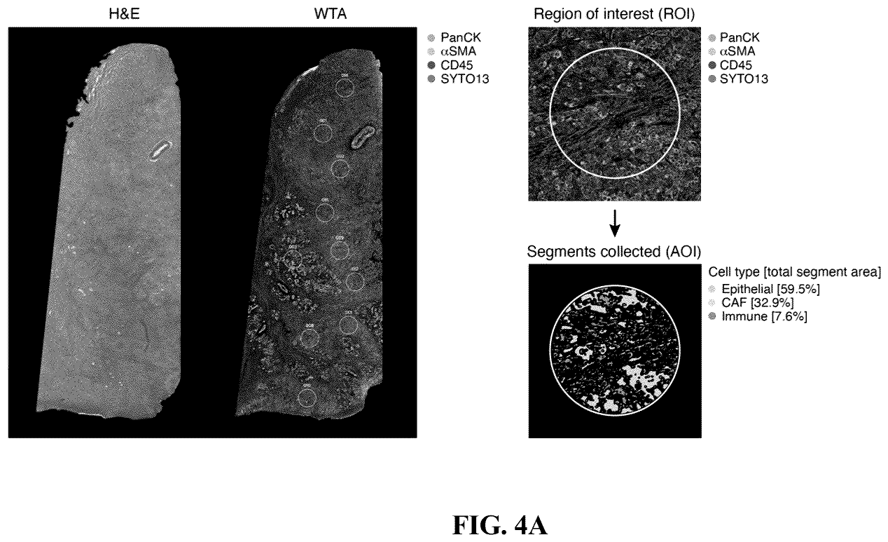

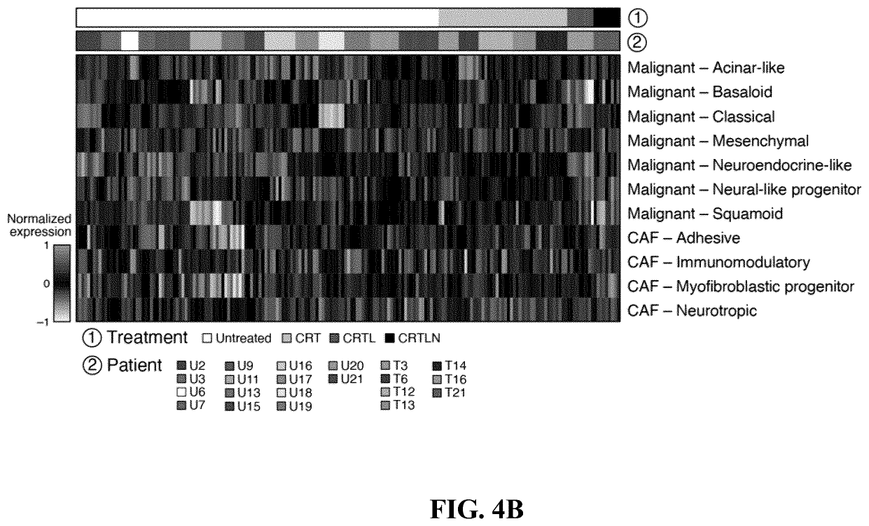

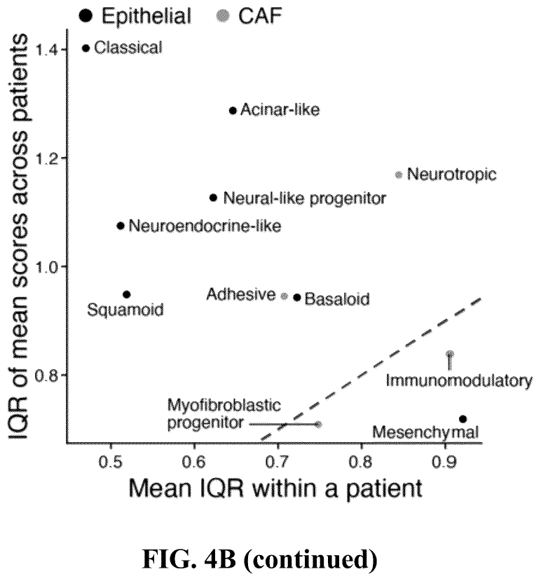

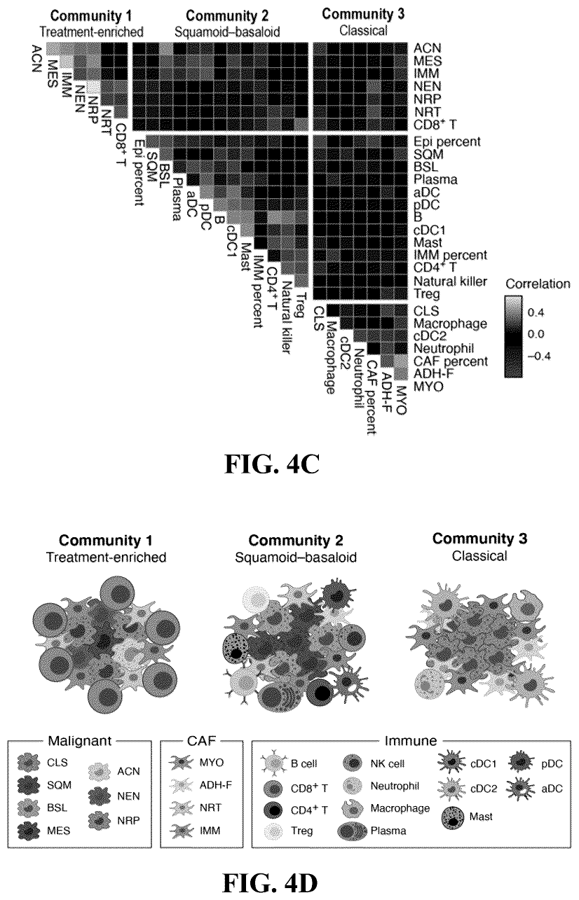

In some embodiments, a PDAC tumor can comprise a tumor spatial community. Such a tumor spatial community can be enriched (or depleted) with cells of different types, states, expression signatures and/or programs, or combinations thereof. In certain exemplary embodiments, the tumor spatial community can have a community composition of that set forth in any of e.g., FIGS. 4B-4D. In some embodiments, the tumor spatial community is a treatment-enriched community; a squamoid-basaloid community; or a classical community.

In certain example embodiments, the treatment-enriched community is enriched with cell(s) expressing a malignant cell lineage program selected from a neural-like progenitor program, a neuroendocrine-like program; a mesenchymal program, or an acinar-like program, or a combination thereof; cell(s) expressing a CAF expression program selected from a neurotropic program, an immunomodulatory program, or both; CD8+ T-cells; or any combination thereof.

In certain example embodiments, the squamoid-basaloid community is enriched with cell(s) expressing a malignant cell linage program selected from a squamoid program or a basaloid program, cells expressing a CAF immunomodulatory program, CD4+ T cells, B cells, regulatory T cells, natural killer cells, mast cells, conventional type 1 dendritic cells, plasmacytoid dendritic (pDC) cells, activated dendritic (aDC) cells, plasma cells.

In certain example embodiments, the classical community is enriched with cell(s) expressing a CAF myofibroblastic progenitor program, cell(s) expressing a CAF adhesive program, cell(s) expressing a malignant lineage classical-like program, macrophages, conventional type 2 dendritic cells, or any combination thereof.

In certain example embodiments, the tumor spatial community is enriched in cell(s) expressing a neuroendocrine-like program and/or a neural like malignant cell lineage program, CD8+ T cells, and conventional type 2 dendritic cells.

In certain example embodiments, the tumor spatial community is depleted of conventional type 1 dendritic cells.

### Methods of Detecting Expression Signatures and Programs

Described herein are methods of detecting one or more signatures and/or programs, such as a PDAC signature and/or program, in one or more tissues and/or cells of a subject. In some embodiments, a PDAC signature and/or program is detected in a single cell of a PDAC tumor. In some embodiments, a PDAC signature and/or program is detected in a single nucleus of a PDAC tumor cell or PDAC tumor-associated cell. In some embodiments, a tumor-associated cell is an immune cell present in a tumor microenvironment (i.e., the microenvironment surrounding the tumor in situ) and/or tumor niche local microenvironment (i.e., a specific region or compartment within a tumor). PDAC signatures and/or programs that can be detected in various embodiments are discussed and described in greater detail elsewhere herein.

In one embodiment, the signature's and/or program's genes, biomarkers, and/or cells may be detected or isolated by immunofluorescence, immunohistochemistry (IHC), fluorescence activated cell sorting (FACS), mass spectrometry (MS), mass cytometry (CyTOF), any gene or transcript sequencing method, including but not limited to, RNA-seq, single cell RNA-seq, single nucleus RNAseq, spatial transcriptomics, spatial proteomics, quantitative RT-PCR, single cell qPCR, FISH, RNA-FISH, MERFISH (multiplex (in situ) RNA FISH), Nanostring, in situ hybridization, CRISPR-effector system mediated screening assay (e.g. SHERLOCK assay), compressed sensing, and any combination thereof. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26 (3): 317-25). These and other methods are described in greater detail elsewhere herein (see e.g., the section regarding “methods of diagnosing, prognosing, and/or treating PDAC” and Working Examples herein).

Methods of Diangosing, Classifying, Prognosing, and/or Treating PDAC

Described herein are methods of diagnosing, prognosing, and/or treating PDAC in a subject in need thereof. In some embodiments, methods of diagnosing, prognosing, and/or treating PDAC in a subject in need thereof can include detecting one or more PDAC signatures and/or programs, which are described in greater detail elsewhere herein.

In some embodiments, the method includes detecting one or more signatures and/or programs, such as a PDAC signature and/or programs, in one or more tissues and/or cells of a subject. In some embodiments, a PDAC signature and/or program is detected in a single cell of a PDAC tumor. In some embodiments, a PDAC signature and/or program is detected in a single nucleus of a PDAC tumor cell or PDAC tumor-associated cell. In some embodiments, a tumor-associated cell is an immune cell present in a tumor microenvironment (i.e., the microenvironment surrounding the tumor in situ) and/or tumor niche local microenvironment (i.e., a specific region or compartment within a tumor). PDAC signatures and/or programs that can be detected in various embodiments are discussed and described in greater detail elsewhere herein.

In one embodiment, the signature's and/or program's genes, biomarkers, and/or cells may be detected or isolated by immunofluorescence, immunohistochemistry (IHC), fluorescence activated cell sorting (FACS), mass spectrometry (MS), mass cytometry (CyTOF), any gene or transcript sequencing method, including but not limited to, RNA-seq, single cell RNA-seq, single nucleus RNAseq, spatial transcriptomics, spatial proteomics, quantitative RT-PCR, single cell qPCR, FISH, RNA-FISH, MERFISH (multiplex (in situ) RNA FISH), Nanostring, in situ hybridization, CRISPR-effector system mediated screening assay (e.g., SHERLOCK assay), compressed sensing, and any combination thereof. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26 (3): 317-25). These and other methods are described in greater detail elsewhere herein (see e.g., the section regarding “methods of diagnosing, prognosing, and/or treating PDAC” and Working Examples herein).

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse). In certain embodiments, the present invention provides for one or more therapeutic agents against combinations of targets identified. Targeting the identified combinations may provide for enhanced or otherwise previously unknown activity in the treatment of disease.

Described in certain example embodiments are methods of diagnosing, classifying and/or prognosing pancreatic ductal adenocarcinoma (PDAC), optionally time to progression (TTP) and/or overall survival (OS), in a subject in need thereof, comprising diagnosing the PDAC in the subject in need thereof, wherein diagnosing comprises detecting, in one or more PDAC tumor cells or organoids derived therefrom, (i) a malignant cell signature, program or both; (ii) a cancer-associated fibroblast (CAF) signature, program, or both; (iii) a tumor spatial community; (iv) one or more co-expressed receptor-ligand pairs; or (v) any combination thereof; wherein diagnosing, classifying and/or prognosing the PDAC is determined based on detection of one or more of the signatures, programs, communities, or receptor-ligand pairs.

Described in certain example embodiments herein are methods of treating pancreatic ductal adenocarcinoma (PDAC) in a subject in need thereof, comprising diagnosing, classifying, and/or prognosing the PDAC in the subject in need thereof, wherein diagnosing comprises detecting, in one or more PDAC tumor cells or organoids derived therefrom (i) a malignant cell signature, program or both; (ii) a cancer-associated fibroblast (CAF) signature, program, or both; (iii) a tumor spatial community; (iv) one or more co-expressed receptor-ligand pairs; or (v) any combination thereof; wherein diagnosing, classifying, and/or prognosing the PDAC is determined based on detection of one or more of the signatures, programs, communities, or receptor-ligand pairs; and administering, a PDAC treatment to the subject in need thereof, wherein the treatment optionally comprises a PDAC malignant cell modulating agent, a CAF modulating agent, an immune modulator, an apoptosis inhibitor, a TGFbeta modulator, a CXCR4 inhibitor, a HER2 inhibitor, or any combination thereof to the subject, wherein the PDAC treatment administered is based at least in part on the diagnosis, classification, and/or prognosis of the PDAC.

In certain example embodiments, the immune modulator is a myeloid cell agonist or antagonist.

In certain example embodiments, the PDAC malignant cell modulating agent and/or CAF modulating agent comprise a therapeutic antibody or fragment/combination thereof, antibody-like protein scaffold, aptamer, polypeptide, a polynucleotide, a genetic modifying agent or system, a small molecule therapeutic, a chemotherapeutic, small molecule degrader, inhibitor, an immunomodulator, or a combination thereof.

In certain example embodiments, the malignant cell signature or program comprises (i) a lineage specific expression program selected from a squamoid program, a mesenchymal program, a basaloid program, a classical-like program, an acinar-like program, a neuroendocrine-like program, a neural-like progenitor program, or any combination thereof; (ii) a cell state specific expression selected from a cycling(S) program, a cycling (G2/M) program, a TNF-NFkB signaling program, a MYC signaling program, an adhesive program, a ribosomal program, an interferon signaling program, or a combination thereof; (iii) a neoadjuvant treated malignant cell expression program; (iv) an untreated malignant cell expression program; or (v) any combination thereof.

In certain example embodiments, the neural-like progenitor program comprises one or more drug efflux programs and/or genes, apoptosis regulation programs and/or genes, chemoresistance programs and/or genes, tumor-nerve cross-talk programs and/or genes, neuronal gene expression programs, or neuronal development/migration/adhesion programs and/or genes, tissue stem cell module programs and/or genes, organ morphogenesis programs and/or genes, or hepatocyte nuclear factor activity programs and/or genes.

In certain example embodiments, the neural-like progenitor program comprises one or more genes selected from: CNTN4, CTNND2, NRXN3, RELN, SEMASA, NRCAM, AUTS2, ABCB1, BCL2, PDGFD, SPP1, SEMA3E, NFIB; any one or more genes in Table 5; any one or more genes in FIG. 15.

In certain example embodiments, the PDAC treatment inhibits or prevents, in one or more cells, expression of a malignant lineage program selected from a neural-like progenitor program, neuroendocrine-like program, basaloid program, mesenchymal program, or any combination thereof; an adhesive malignant state expression program; and/or a fibroblast adhesive program, or any combination thereof.

In certain example embodiments, the CAF signature or program comprises (i) a cell state specific expression program selected from an adhesive program, an immunomodulatory program, a myofibroblastic progenitor program, a neurotropic program, or a combination thereof; (ii) a neoadjuvant treated CAF expression program; (iii) an untreated CAF expression program; or (iv) any combination thereof.

In certain example embodiments, the PDAC treatment inhibits or prevents expression of a CAF adhesive program in one or more cells.

In certain example embodiments, the neoadjuvant treated malignant cell expression program comprises a malignant cell lineage program selected from a neural-like progenitor program, a neuroendocrine-like program, a mesenchymal program, a basaloid program, or a combination thereof; an adhesive malignant state expression program; or any combination thereof.

In certain example embodiments, the neoadjuvant treated CAF expression program comprises a fibroblast adhesive program.

In certain example embodiments, the tumor spatial community is a treatment-enriched community; a squamoid-basaloid community; or a classical community.

In certain example embodiments, the treatment-enriched community is enriched with cell(s) expressing a malignant cell lineage program selected from a neural-like progenitor program, a neuroendocrine-like program; a mesenchymal program, or an acinar-like program, or a combination thereof; cell(s) expressing a CAF expression program selected from a neurotropic program, an immunomodulatory program, or both; CD8+ T-cells; or any combination thereof.

In certain example embodiments, the squamoid-basaloid community is enriched with cell(s) expressing a malignant cell linage program selected from a squamoid program or a basaloid program, cells expressing a CAF immunomodulatory program, CD4+ T cells, B cells, regulatory T cells, natural killer cells, mast cells, conventional type 1 dendritic cells, plasmacytoid dendritic (pDC) cells, activated dendritic (aDC) cells, plasma cells.

In certain example embodiments, the classical community is enriched with cell(s) expressing a CAF myofibroblastic progenitor program, cell(s) expressing a CAF adhesive program, cell(s) expressing a malignant lineage classical-like program, macrophages, conventional type 2 dendritic cells, or any combination thereof.

In certain example embodiments, the tumor spatial community is enriched in cell(s) expressing a neuroendocrine-like program and/or a neural like malignant cell lineage program, CD8+ T cells, and conventional type 2 dendritic cells.

In certain example embodiments, the tumor spatial community is depleted of conventional type 1 dendritic cells.

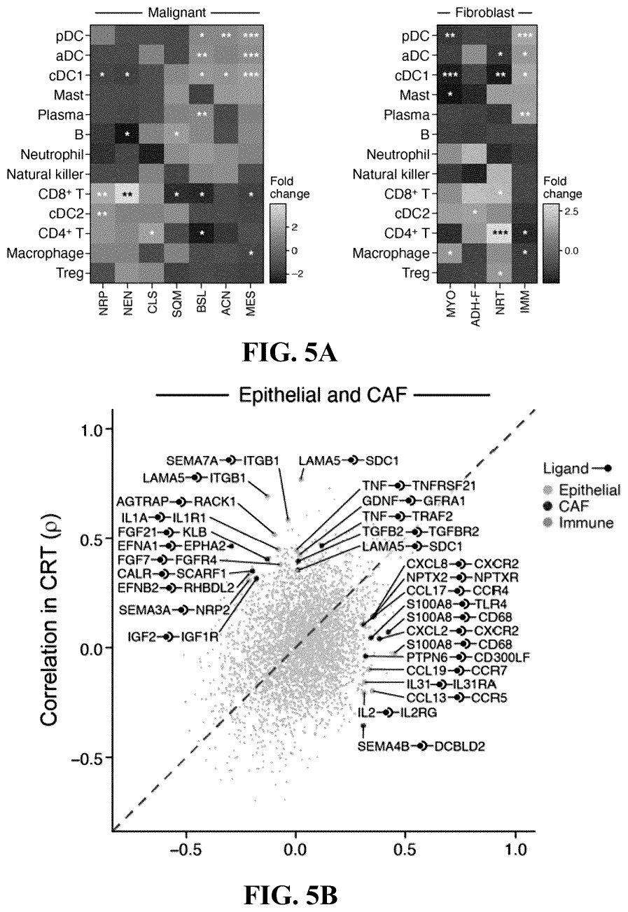

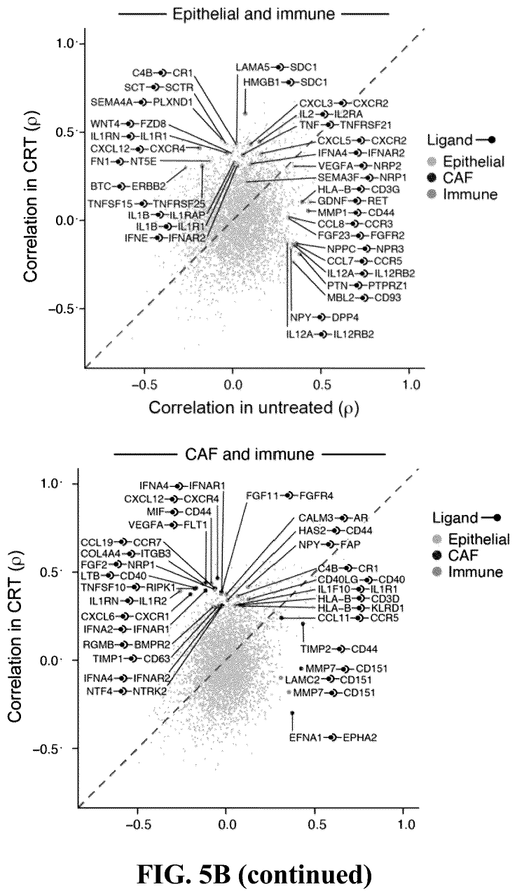

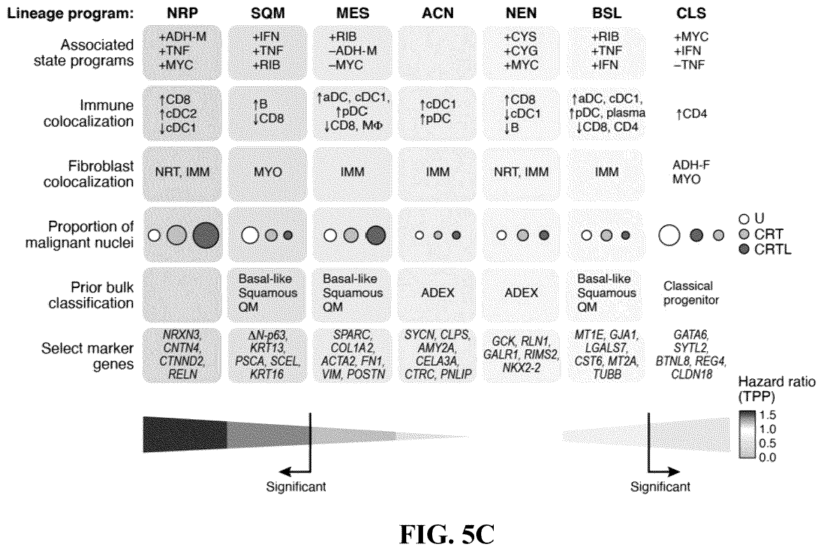

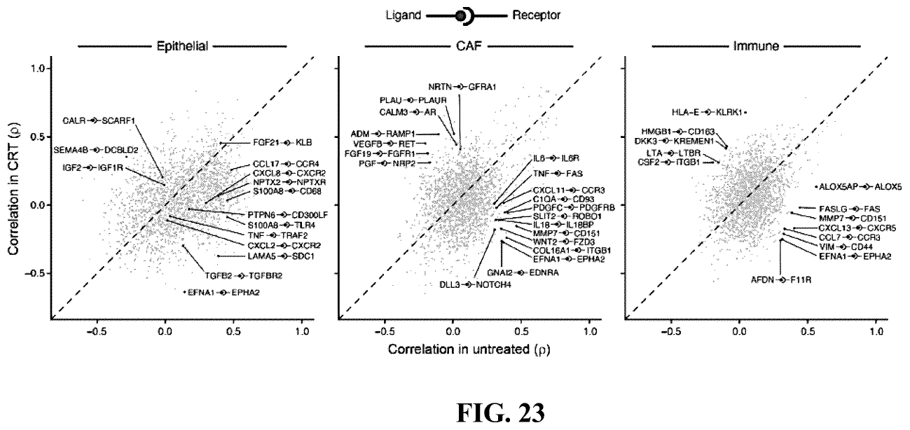

In certain example embodiments, the one or more co-expressed receptor-ligand pairs are selected from FIG. 5b, FIG. 23, Table 3, or any combination thereof.

In certain example embodiments the method further comprises prognosing PDAC, optionally time to progression (TTP) and/or overall survival (OS), in the subject in need thereof, wherein prognosing is based at least in part on detection of one or more of the signatures, programs, communities, or receptor-ligand pairs.

In certain example embodiments, the TTP is predicted to be shorter for subjects with PDAC tumors or organoids derived therefrom expressing a malignant cell neural-like progenitor program and/or a squamoid program.

In certain example embodiments, the TTP is predicted to be longer for subjects with PDAC tumors or organoids derived therefrom expressing a malignant cell classical-like program and/or a CAF immunomodulatory program.

In certain example embodiments, the OS is predicted to be shorter for subjects with PDAC tumors or organoids derived therefrom expressing a malignant cell neural-like progenitor program and/or a squamoid program; and/or expressing a CAF adhesive program.

In certain example embodiments, the OS is predicted to be longer for subjects with PDAC tumors or organoids derived therefrom expressing a malignant cell classical-like program.

In certain example embodiments, the subject has had or is concurrently receiving a neoadjuvant therapy.

In certain example embodiments, detecting comprises a single cell RNA sequencing technique.

In certain example embodiments, detecting comprises a single-nucleus RNA sequencing technique.

In certain example embodiments, the single-nucleus RNA sequencing technique is optimized for pancreatic tissue.

In certain example embodiments, the single-nucleus RNA sequencing technique is optimized for frozen tissue.

In certain example embodiments, detecting comprises a spatially-resolved transcriptomics technique.

In certain example embodiments, the single-nucleus RNA sequencing technique comprises screening a sample for an RNA integrity number and performing single nucleus RNA sequencing only on samples with an RNA integrity number of 6 or more.

In certain example embodiments, detecting comprises a spatially-resolved transcriptomics technique.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub) population. Furthermore, the signature may be indicative of cells within a population of cells in vivo. The signature may also be used to suggest for instance particular therapies, or to follow up treatment, or to suggest ways to modulate immune systems. The signatures of the present invention may be discovered by analysis of expression profiles of single cells within a population of cells from isolated samples (e.g., Sys tumor samples), thus allowing the discovery of novel cell subtypes or cell states that were previously invisible or unrecognized. The presence of subtypes or cell states may be determined by subtype specific or cell state specific signatures. The presence of these specific cell (sub) types or cell states may be determined by applying the signature genes to bulk sequencing data in a sample. In certain embodiments, the signatures of the present invention may be microenvironment specific, such as their expression in a particular spatio-temporal context. In certain embodiments, signatures as discussed herein are specific to a particular pathological context. In certain embodiments, a combination of cell subtypes having a particular signature may indicate an outcome. In certain embodiments, the signatures can be used to deconvolute the network of cells present in a particular pathological condition. In certain embodiments, the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of cells that are linked to particular pathological condition (e.g., inflammation), or linked to a particular outcome or progression of the disease or linked to a particular response to treatment of the disease.

The invention provides biomarkers (e.g., phenotype specific or cell type) for the identification, diagnosis, prognosis and manipulation of cell properties, for use in a variety of diagnostic and/or therapeutic indications. Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein.

Biomarkers are useful in methods of diagnosing, prognosing and/or staging an immune response in a subject by detecting a first level of expression, activity and/or function of one or more biomarker and comparing the detected level to a control of level wherein a difference in the detected level and the control level indicates that the presence of an immune response in the subject.

The terms “diagnosis” and “monitoring” are commonplace and well-understood in medical practice. By means of further explanation and without limitation the term “diagnosis” generally refers to the process or act of recognizing, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The biomarkers of the present invention are useful in methods of identifying patient populations at risk or suffering from an immune response based on a detected level of expression, activity and/or function of one or more biomarkers. These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments.

The term “monitoring” generally refers to the follow-up of a disease or a condition in a subject for any changes which may occur over time.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

In some embodiments, an altered quality, quantity, and/or phenotype of PDAC tumor cells in or from the subject compared to a suitable control or reference value(s) can indicate that the subject would benefit from or is in need of a specific treatment. In some of such embodiments, the method can further include administration of such a specifically identified treatments.

In some embodiments, an altered quality, quantity, and/or phenotype of PDAC tumor cells in or from the subject compared to a suitable control or reference value(s) can indicate that the subject falls into a particular group or subset of patients all diagnosed with or having the same general disease (e.g. cancer, pancreatic cancer, PDAC, etc.), where each group optionally can be treated in different ways specific to each group to improve outcome, as well as, improve general patient care by allowing greater precision prediction of individual patient survival and/or treatment response.

The methods described herein can rely on comparing the quantity or quality of PDCA tumor cell population cell populations, biomarkers, or gene or gene product signatures measured in samples from patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterised by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1xSD or ±2xSD or ±3xSD, or ±1xSE or ±2xSE or ±3xSE). Deviation may also refer to a value falling outside of a reference range defined by values in a given population (for example, outside of a range which comprises ≥40%, ≥50%, ≥60%, ≥70%, ≥75%, or ≥80%, or ≥85%, or ≥90%, or ≥95%, or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR-), Youden index, or similar.

In one embodiment, the signature genes, biomarkers, and/or cells may be detected or isolated by immunofluorescence, immunohistochemistry (IHC), fluorescence activated cell sorting (FACS), mass spectrometry (MS), mass cytometry (CyTOF), any gene or transcript sequencing method, including but not limited to, RNA-seq, single cell RNA-seq, single nucleus RNAseq, spatial transcriptomics, spatial proteomics, quantitative RT-PCR, single cell qPCR, FISH, RNA-FISH, MERFISH (multiplex (in situ) RNA FISH), in situ hybridization, CRISPR-effector system mediated screening assay (e.g., SHERLOCK assay), compressed sensing, and any combination thereof. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26 (3): 317-25).

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS) n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g., diabodies etc.) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies. Immunoassays have been designed for use with a wide range of biological sample matrices. Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (1125) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Single Cell RNA Sequencing

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi: 10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. Jan; 12 (1): 44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14 (3): 302-308, 2017; Cao, et al., Comprehensive single-cell transcriptional profiling of a multicellular organism. Science, 357 (6352): 661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14 (10): 955-958; International patent application number PCT/US2016/059239, published as WO2017164936 on Sep. 28, 2017; International patent application number PCT/US2018/060860, published as WO/2019/094984 on May 16, 2019; International patent application number PCT/US2019/055894, published as WO/2020/077236 on Apr. 16, 2020; and Drokhlyansky, et al., “The enteric nervous system of the human and mouse colon at a single-cell resolution,” bioRxiv 746743; doi: doi.org/10.1101/746743, which are herein incorporated by reference in their entirety. In some embodiments the snRNA-seq method is optimized for a pancreatic sample. In some embodiments the snRNA-seq method is optimized for a frozen sample. In some embodiments, the snRNA-seq is optimized for a frozen pancreatic sample. In some embodiments, the snRNA-seq method comprises determining an RNA integrity number of a sample. In some embodiments, the snRNA-seq method comprises using only samples with an RNA integrity number of 6 or greater or greater than 6. Additional details can be found in the Working Examples elsewhere herein.

In certain embodiments, the invention involves the Assay for Transposase Accessible Chromatin using sequencing (ATAC-seq) as described. See e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348 (6237): 910-4. doi: 10.1126/science.aab1601. Epub 2015 May 7; U.S. Pat. No. 20,160,208323A1; U.S. Pat. No. 20,160,060691A1; and WO2017156336A1).

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854,5,288,644, 5,324,633, 5,432,049, 5,470,710, 5,492,806, 5,503,980, 5,510,270, 5,525,464, 5,547,839, 5,580,732, 5,661,028, 5,800,992, the disclosures of which are incorporated herein by reference, as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-Interscience, NY (1987), which is incorporated in its entirety for all purposes. When the cDNA microarrays are used, typical hybridization conditions are hybridization in 5×SSC plus 0.2% SDS at 65C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization with Nucleic Acid Probes “, Elsevier Science Publishers B.V. (1993) and Kricka, “Nonisotopic DNA Probe Techniques”, Academic Press, San Diego, Calif. (1992).

### Compressed Sensing

Mammalian genomes contain approximately 20,000 genes, and mammalian expression profiles are frequently studied as vectors with 20,000 entries corresponding to the abundance of each gene. It is often assumed that studying gene expression profiles requires measuring and analyzing these 20,000 dimensional vectors, but some mathematical results show that it is often possible to study high-dimensional data in low dimensional space without losing much of the pertinent information. In one embodiment of the present invention, less than 20,000 aptamers are used to detect protein expression in single cells. Not being bound by a theory, working in low dimensional space offers several advantages with respect to computation, data acquisition and fundamental insights about biological systems.

In one embodiment, aptamers are chosen for protein targets that are generally part of gene modules or programs, whereby detection of a protein allows for the ability to infer expression of other proteins present in a module or gene program. Samples are directly compared based only on the measurements of these signature genes.

In alternative embodiments, sparse coding or compressed sensing methods can be used to infer large amounts of data with a limited set of target proteins. Not being bound by a theory, the abundance of each of the 20,000 genes can be recovered from random composite measurements. In this regard, reference is made to Cleary et al., “Composite measurements and molecular compressed sensing for highly efficient transcriptomics” posted on Jan. 2, 2017 at biorxiv.org/content/early/2017/01/02/091926, doi.org/10.1101/091926, incorporated herein by reference in its entirety.

In some embodiments, the method of diagnosing, prognosing, and/or monitoring, can include obtaining a sample, such as a PDCA tumor sample, and analyzing cell signatures from cells in bulk or individually by one or more methods described herein. In some embodiments, the method includes analyzing PDCA tumor sample using snRNA-seq and/or spatial transcriptomics. In some embodiments, the tumor sample is obtained before resection, such as by biopsy. In some embodiments, the tumor sample is obtained after tumor resection.

In some embodiments, for example, a tissue sample may be obtained and analyzed for specific cell markers (IHC) or specific transcripts (e.g., RNA-FISH). Tissue samples for diagnosis, prognosis or detecting may be obtained by endoscopy. In one embodiment, a sample may be obtained by endoscopy and analyzed by FACS. As used herein, “endoscopy” refers to a procedure that uses an endoscope to examine the interior of a hollow organ or cavity of the body. The endoscope may include a camera and a light source. The endoscope may include tools for dissection or for obtaining a biological sample. A cutting tool can be attached to the end of the endoscope, and the apparatus can then be used to perform surgery. Applications of endoscopy that can be used with the present invention include, but are not limited to examination of the esophagus, stomach and duodenum (esophagogastroduodenoscopy); small intestine (enteroscopy); large intestine/colon (colonoscopy, sigmoidoscopy); bile duct; rectum (rectoscopy) and anus (anoscopy), both also referred to as (proctoscopy); respiratory tract; nose (rhinoscopy); lower respiratory tract (bronchoscopy); ear (otoscope); urinary tract (cystoscopy); female reproductive system (gynoscopy); cervix (colposcopy); uterus (hysteroscopy); fallopian tubes (falloposcopy); normally closed body cavities (through a small incision); abdominal or pelvic cavity (laparoscopy); interior of a joint (arthroscopy); or organs of the chest (thoracoscopy and mediastinoscopy).

### Adoptive Cell Transfer

In some embodiments, a method of treatment can include treatment with adoptive cell transfer.

As used herein, “ACT”, “adoptive cell therapy” and “adoptive cell transfer” may be used interchangeably. In certain embodiments, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8 (1): 424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June; 24 (6): 724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314 (5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32:189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12 (4): 269-281; and Jenson and Riddell, 2014, Design and implementation of adoptive therapy with chimeric antigen receptor-modified T cells. Immunol Rev. 257 (1): 127-144; and Rajasagi et al., 2014, Systematic identification of personal tumor-specific neoantigens in chronic lymphocytic leukemia. Blood. 2014 Jul. 17; 124 (3): 453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor RORI; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RUI, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac (2-8) aNeu5Ac (2-3) bDGalp (1-4) bDGlcp (1-1) Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis (Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac (2-3) bDGalp (1-4) bDGlcp (1-1) Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEMI/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SARTI, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1,-2,-3 or-4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAPI (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2,-3, 4); FBP (folate binding protein);, fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); LICAM (LI cell adhesion molecule); MCIR (melanocortin 1 receptor); Myosin/m (myosin mutated); MUM-1,-2,-3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukaemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (DI), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WTI, CD22, CD171, RORI, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CML), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR α and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753, 162; 8,211,422; and, PCT Publication WO9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8α transmembrane domain, to the transmembrane and intracellular signaling domains of either CD35 or FcRγ (scFv-CD3ζ or scFv-FcRγ; see U.S. Pat. Nos. 7,741,465; 5,912,172; and 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD32; see U.S. Pat. Nos. 8,911,993; 8,916,381; 8,975,071; 9,101,584; 9,102,760; and 9,102,761). Third-generation CARs include a combination of costimulatory endodomains, such a CD3ζ-chain, CD97, GDI la-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO 2014/134165; PCT Publication No. WO 2012/079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon R1b), CD79a, CD79b, Fc gamma RIIa, DAP10, and DAP12. In certain preferred embodiments, the primary signaling domain comprises a functional signaling domain of CD3ζ or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAMI (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of 4-1BB, CD27, and CD28. In certain embodiments, a chimeric antigen receptor may have the design as described in U.S. Pat. No. 7,446,190, comprising an intracellular domain of CD3ζ chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139 (sequence version 1, 2 or 3):

(SEQ ID NO: 1)
IEVMYPPPYLDNEKSNGTIIHVKGKHLCPSPLFPGPSKPFWVLVVVGGV
LACYSLLVTVAFIIFWVRSKRSRLLHSDYMNMTPRRPGPTRKHYQPYAP
PRDFAAYRS)).

Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD33 chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects.

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34:1157-1165), a portion of the human CD28 molecule, and the intracellular component of the human TCR-2 molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY (SEQ. I.D. No. 2) and continuing all the way to the carboxy-terminus of the protein. To encode the anti-CD19 scFv component of the vector, the authors designed a DNA sequence which was based on a portion of a previously published CAR (Cooper et al., (2003) Blood 101:1637-1644). This sequence encoded the following components in frame from the 5′ end to the 3′ end: an Xhol site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with Xhol and NotI. To form the MSGV-FMC63-28Z retroviral vector, the Xhol and NotI-digested fragment encoding the FMC63 scFv was ligated into a second Xhol and NotI-digested fragment that encoded the MSGV retroviral backbone (as in Hughes et al., (2005) Human Gene Therapy 16:457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20:70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain, such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM 006139 (sequence version 1, 2 or 3) starting with the amino acid sequence IEVMYPPPY (SEQ ID NO: 2) and continuing all the way to the carboxy-terminus of the protein. The sequence is reproduced herein:

(SEQ ID NO: 1)
IEVMYPPPYLDNEKSNGTIIHVKGKHLCPSPLFPGPSKPFWVLVVVGGV
LACYSLLVTVAFIIFWVRSKRSRLLHSDYMNMTPRRPGPTRKHYQPYAP
PRDFAAYRS.

Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in International Patent Publication No. WO 2015/187528. More particularly, Example 1 and Table 1 of WO 2015/187528, incorporated by reference herein, demonstrate the generation of anti-CD19 CARs based on a fully human anti-CD19 monoclonal antibody (47G4, as described in US Patent Publication No. 2010/0104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signaling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-27-FcεRI gamma chain; or CD28-FcεRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO 2015/187528 and an intracellular T-cell signaling domain as set forth in Table 1 of WO 2015/187528. Preferably, the antigen is CD19, more preferably the antigen-binding element is an anti-CD19 scFv, even more preferably the mouse or human anti-CD19 scFv as described in Example 1 of WO 2015/187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO 2015/187528.

By means of an example and without limitation, chimeric antigen receptor that recognizes the CD70 antigen is described in International Patent Publication No. WO 2012/058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20 (1): 55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am.J.Pathol. 1995; 147:1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US Patent Publication No. 2016/0046724 A1; International Patent Publication Nos. WO 2016/014789 A2, WO 2017/211900 A1, WO 2015/158671 A1, WO2018028647A1, and WO 2013/154760 A1; and US Patent Publication Nos. 2018/0085444 A1 and 2017/0283504 A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., International Patent Publication Nos. WO 2013/044225, WO 2016/000304, WO 2015/057834, WO 2015/057852, WO 2016/070061, U.S. Pat. No. 9,233,125, and US Patent Publication No. 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US Patent Publication Nos. 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLOS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In certain embodiments, ACT includes co-transferring CD4+ Th1 cells and CD8+ CTLs to induce a synergistic antitumor response (see, e.g., Li et al., Adoptive cell therapy with CD4+ T helper 1 cells and CD8+ cytotoxic T cells enhances complete rejection of an established tumor, leading to generation of endogenous memory responses to non-targeted tumor epitopes. Clin Transl Immunology. 2017 October; 6 (10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112 (2): 362-73; and Martin-Orozco N, et al., T helper 17 cells promote cytotoxic T cell activation in tumor immunity. Immunity. 2009 Nov. 20; 31 (5): 787-98). Those studies involved an adoptive T cell transfer (ACT) therapy approach, which takes advantage of CD4⁺ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257 (1): 56-71. doi: 10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3 (10): 1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells or population of cells may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e., intracavity delivery) or directly into a tumor prior to resection (i.e., intratumoral delivery). In one embodiment, the cell compositions of the present invention are preferably administered by intravenous injection.

The administration of the cells or population of cells can consist of the administration of 104-109 cells per kg body weight, preferably 105 to 106 cells/kg body weight including all integer values of cell numbers within those ranges. Dosing in CAR T cell therapies may for example involve administration of from 106 to 109 cells/kg, with or without a course of lymphodepletion, for example with cyclophosphamide. The cells or population of cells can be administrated in one or more doses. In another embodiment, the effective amount (e.g., number) of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be done directly by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6:95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 2013/0071414; PCT Patent Publication Nos. WO 2011/146862, WO 2014/011987, WO 2013/040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28 (6): 1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23 (9): 2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 Jan. 25; 9 (374); Legut, et al., 2018, CRISPR-mediated TCR replacement generates superior anticancer transgenic T cells. Blood, 131 (3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see PCT Patent Publications: WO2013176915, WO2014059173, WO2014172606, WO2014184744, and WO2014191128).

In certain embodiments, editing may result in inactivation of a gene. By inactivating a gene, it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells (such as by CRISPR/Cas), particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543:113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, a and B, which assemble to form a heterodimer and associates with the CD3-transducing subunits to form the T cell receptor complex present on the cell surface. Each a and B chain of the TCR consists of an immunoglobulin-like N-terminal variable (V) and constant (C) region, a hydrophobic transmembrane domain, and a short cytoplasmic region. As for immunoglobulin molecules, the variable region of the a and B chains are generated by V (D) J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells (such as by CRISPR/Cas), particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112 (12): 4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells (such as by CRISPR/Cas), particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44 (2): 356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MTI and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIRI, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in WO201704916).

In certain embodiments, editing of cells (such as by CRISPR/Cas), particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUCI, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in WO2016011210 and WO2017011804).

In certain embodiments, editing of cells (such as by CRISPR/Cas), particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PDI simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead: cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells are minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US20040224402 entitled, Generation and Isolation of Antigen-Specific T Cells, or in U.S. Pat. Nos. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g., via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274 (5284): 94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of ¹²⁵I labeled β2-microglobulin (β2m) into MHC class I/β2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in WO2015120096, by a method comprising: enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in WO2016191756 comprising administering to the patient a dose of cyclophosphamide between 200 mg/m2/day and 2000 mg/m2/day and a dose of fludarabine between 20 mg/m2/day and 900 mg/m²/day.

### Modulating PDAC Signatures and Modulating Agents

In some embodiments, the method, such a method of treatment, includes modulating a PDAC signature, or, maintaining (i.e., preventing a shift in signature away from a desired signature) a desired PDAC signature. In general, such methods include administering a modulating agent to a subject. In some embodiments, the treatment comprises a PDAC malignant cell modulating agent, a CAF modulating agent, an immune modulator, a TGFbeta modulator, and/or other modulating agents described in greater detail elsewhere herein. In certain example embodiments, the immune modulator is a myeloid cell agonist or antagonist. In certain example embodiments, the PDAC malignant cell modulating agent and/or CAF modulating agent comprise a therapeutic antibody or fragment/combination thereof, antibody-like protein scaffold, aptamer, polypeptide, a polynucleotide, a genetic modifying agent or system, a small molecule therapeutic, a chemotherapeutic, small molecule degrader, inhibitor, an immunomodulator, or a combination thereof.

### Exemplary Modulating Agents

As used herein, “modulating” or “to modulate” generally means either reducing or inhibiting the expression or activity of, or alternatively increasing the expression or activity of a target or antigen. In particular, “modulating” or “to modulate” can mean either reducing or inhibiting the activity of, or alternatively increasing a (relevant or intended) biological activity of, a target or antigen as measured using a suitable in vitro, cellular or in vivo assay (which will usually depend on the target involved), by at least 5%, at least 10%, at least 25%, at least 50%, at least 60%, at least 70%, at least 80%, at least 90%, or more, compared to activity of the target in the same assay under the same conditions but without the presence of an agent. An “increase” or “decrease” refers to a statistically significant increase or decrease respectively. For the avoidance of doubt, an increase or decrease will be at least 10% relative to a reference, such as at least 10%, at least 20%, at least 30%, at least 40%, at least 50%, at least 60%, at least 70%, at least 80%, at least 90%, at least 95%, at least 97%, at least 98%, or more, up to and including at least 100% or more, in the case of an increase, for example, at least 2-fold, at least 3-fold, at least 4-fold, at least 5-fold, at least 6-fold, at least 7-fold, at least 8-fold, at least 9-fold, at least 10-fold, at least 50-fold, at least 100-fold, or more. “Modulating” can also involve effecting a change (which can either be an increase or a decrease) in affinity, avidity, specificity and/or selectivity of a target or antigen. “Modulating” can also mean effecting a change with respect to one or more biological or physiological mechanisms, effects, responses, functions, pathways or activities in which the target or antigen (or in which its substrate(s), ligand(s) or pathway(s) are involved, such as its signaling pathway or metabolic pathway and their associated biological or physiological effects) is involved. Again, as will be clear to the skilled person, such an action as an agonist or an antagonist can be determined in any suitable manner and/or using any suitable assay known or described herein (e.g., in vitro or cellular assay), depending on the target or antigen involved.

Modulating can, for example, also involve allosteric modulation of the target and/or reducing or inhibiting the binding of the target to one of its substrates or ligands and/or competing with a natural ligand, substrate for binding to the target. Modulating can also involve activating the target or the mechanism or pathway in which it is involved. Modulating can for example also involve effecting a change in respect of the folding or confirmation of the target, or in respect of the ability of the target to fold, to change its conformation (for example, upon binding of a ligand), to associate with other (sub) units, or to disassociate. Modulating can for example also involve effecting a change in the ability of the target to signal, phosphorylate, dephosphorylate, and the like.

As used herein, an “agent” can refer to a protein-binding agent that permits modulation of activity of proteins or disrupts interactions of proteins and other biomolecules, such as but not limited to disrupting protein-protein interaction, ligand-receptor interaction, or protein-nucleic acid interaction. Agents can also refer to DNA targeting or RNA targeting agents. Agents can also refer to a protein. Agents may include a fragment, derivative and analog of an active agent. The terms “fragment,” “derivative” and “analog” when referring to polypeptides as used herein refers to polypeptides which either retain substantially the same biological function or activity as such polypeptides. An analog includes a proprotein which can be activated by cleavage of the proprotein portion to produce an active mature polypeptide. Such agents include, but are not limited to, antibodies (“antibodies” includes antigen-binding portions of antibodies such as epitope- or antigen-binding peptides, paratopes, functional CDRs; recombinant antibodies; chimeric antibodies; humanized antibodies; nanobodies; tribodies; midibodies; or antigen-binding derivatives, analogs, variants, portions, or fragments thereof), protein-binding agents, nucleic acid molecules, small molecules, recombinant protein, peptides, aptamers, avimers and protein-binding derivatives, portions or fragments thereof. An “agent” as used herein, may also refer to an agent that inhibits expression of a gene, such as but not limited to a DNA targeting agent (e.g., CRISPR system, TALE, Zinc finger protein) or RNA targeting agent (e.g., inhibitory nucleic acid molecules such as RNAi, miRNA, ribozyme).

As used in the context of shifting or modulating a PDAC signature herein, “modulating” also includes maintaining an initial signature (i.e., preventing a shift in signature). As used in the context of shifting or modulating a PDAC signature herein, “modulating agent” includes agents capable of causing a shift in a PDAC signature from an initial signature indicative of a first cell or population state or type to a second signature indicative of a second cell or population state or type, as well as agents capable of maintaining an initial signature. In some embodiments, it may be advantageous to maintain an initial signature, particularly in the context of preventing a shift to a signature that is associated with a less desirable cell or population state or type. As used in this context herein, “modulating agent” is inclusive of pharmaceutical agents (e.g., small molecule compounds, biologics, and the like) that can be administered in a dosage form to a subject as well as physical treatments such as surgical resection, radiation, thermal treatments, and the like that can be applied to a subject and not necessarily in a dosage form. In some embodiments, a modulating agent is administered to a subject before, during, and/or after neoadjuvant treatment and/or PDAC tumor resection.

The agents of the present invention may be modified, such that they acquire advantageous properties for therapeutic use (e.g., stability and specificity), but maintain their biological activity.

It is well known that the properties of certain proteins can be modulated by attachment of polyethylene glycol (PEG) polymers, which increases the hydrodynamic volume of the protein and thereby slows its clearance by kidney filtration. (See, e.g., Clark et al., J. Biol. Chem. 271:21969-21977 (1996)). Therefore, it is envisioned that certain agents can be PEGylated (e.g., on peptide residues) to provide enhanced therapeutic benefits such as, for example, increased efficacy by extending half-life in vivo. In certain embodiments, PEGylation of the agents may be used to extend the serum half-life of the agents and allow for particular agents to be capable of crossing the blood-brain barrier. Thus, in one embodiment, PEGylating inhibitor of HDAC and/or CDK4/6 improve the pharmacokinetics and pharmacodynamics of the inhibitors.

In regard to peptide PEGylation methods, reference is made to Lu et al., Int. J. Pept. Protein Res.43:127-38 (1994); Lu et al., Pept. Res. 6:140-6 (1993); Felix et al., Int. J. Pept. Protein Res. 46:253-64 (1995); Gaertner et al., Bioconjug. Chem. 7:38-44 (1996); Tsutsumi et al., Thromb. Haemost. 77:168-73 (1997); Francis et al., hit. J. Hematol. 68:1-18 (1998); Roberts et al., J. Pharm. Sci. 87:1440-45 (1998); and Tan et al., Protein Expr. Purif. 12:45-52 (1998). Polyethylene glycol or PEG is meant to encompass any of the forms of PEG that have been used to derivatize other proteins, including, but not limited to, mono-(C1-10)alkoxy or aryloxy-polyethylene glycol. Suitable PEG moieties include, for example, 40 kDa methoxy poly(ethylene glycol) propionaldehyde (Dow, Midland, Mich.); 60 kDa methoxy poly(ethylene glycol) propionaldehyde (Dow, Midland, Mich.); 40 kDa methoxy poly(ethylene glycol) maleimido-propionamide (Dow, Midland, Mich.); 31 kDa alpha-methyl-w-(3-oxopropoxy), polyoxyethylene (NOF Corporation, Tokyo); mPEG2-NHS-40k (Nektar); mPEG2-MAL-40k (Nektar), SUNBRIGHT GL2-400MA ((PEG) 240 kDa) (NOF Corporation, Tokyo), SUNBRIGHT ME-200MA (PEG20 kDa) (NOF Corporation, Tokyo). The PEG groups are generally attached to the peptide via acylation or alkylation through a reactive group on the PEG moiety (for example, a maleimide, an aldehyde, amino, thiol, or ester group) to a reactive group on the peptide (for example, an aldehyde, amino, thiol, a maleimide, or ester group).

The PEG molecule(s) may be covalently attached to any Lys, Cys, or K(CO(CH₂)₂SH) residues at any position in a peptide. In certain embodiments, the peptides described herein can be PEGylated directly to any amino acid at the N-terminus by way of the N-terminal amino group. A “linker arm” may be added to a peptide to facilitate PEGylation. PEGylation at the thiol side-chain of cysteine has been widely reported (see, e.g., Caliceti & Veronese, Adv. Drug Deliv. Rev. 55:1261-77 (2003)). If there is no cysteine residue in the peptide, a cysteine residue can be introduced through substitution or by adding a cysteine to the N-terminal amino acid. PEGylaeion can be affected through the side chains of a cysteine residue added to the N-terminal amino acid.

In exemplary embodiments, the PEG molecule(s) may be covalently attached to an amide group in the C-terminus of a peptide. In preferred embodiments, there is at least one PEG molecule covalently attached to the peptide. In certain embodiments, the PEG molecule used in modifying an agent of the present invention is branched while in other embodiments, the PEG molecule may be linear. In particular aspects, the PEG molecule is between 1 kDa and 100 kDa in molecular weight. In further aspects, the PEG molecule is selected from 10, 20, 30, 40, 50, 60, and 80 kDa. In further still aspects, it is selected from 20, 40, or 60 kDa. Where there are two PEG molecules covalently attached to the agent of the present invention, each is 1 to 40 kDa and in particular aspects, they have molecular weights of 20 and 20 kDa, 10 and 30 kDa, 30 and 30 kDa, 20 and 40 kDa, or 40 and 40 kDa. In particular aspects, the agent (e.g., neuromedin U receptor agonists or antagonists) contain mPEG-cysteine. The mPEG in mPEG-cysteine can have various molecular weights. The range of the molecular weight is preferably 5 kDa to 200 kDa, more preferably 5 kDa to 100 kDa, and further preferably 20 kDa to 60 kDA. The mPEG can be linear or branched.

In particular embodiments, the agents include a protecting group covalently joined to the N-terminal amino group. In exemplary embodiments, a protecting group covalently joined to the N-terminal amino group of the agent reduces the reactivity of the amino terminus under in vivo conditions. Amino protecting groups include —C₁₋₁₀ alkyl, —C₁₋₁₀ substituted alkyl, —C₂₋₁₀ alkenyl, —C₂₋₁₀ substituted alkenyl, aryl, —C₁₋₆ alkyl aryl, —C(O)—(CH₂)₁₋₆—COOH, —C(O)—C₁₋₆ alkyl, —C(O)-aryl, —C(O)—O—C₁₋₆ alkyl, or —C(O)—O-aryl. In particular embodiments, the amino terminus protecting group is selected from the group consisting of acetyl, propyl, succinyl, benzyl, benzyloxycarbonyl, and t-butyloxycarbonyl. In other embodiments, deamination of the N-terminal amino acid is another modification that may be used for reducing the reactivity of the amino terminus under in vivo conditions.

Chemically modified compositions of the agents wherein the agent is linked to a polymer are also included within the scope of the present invention. The polymer selected is usually modified to have a single reactive group, such as an active ester for acylation or an aldehyde for alkylation, so that the degree of polymerization may be controlled. Included within the scope of polymers is a mixture of polymers. Preferably, for therapeutic use of the end-product preparation, the polymer will be pharmaceutically acceptable. The polymer or mixture thereof may include but is not limited to polyethylene glycol (PEG), monomethoxy-polyethylene glycol, dextran, cellulose, or other carbohydrate based polymers, poly-(N-vinyl pyrrolidone) polyethylene glycol, propylene glycol homopolymers, a polypropylene oxide/ethylene oxide co-polymer, polyoxyethylated polyols (for example, glycerol), and polyvinyl alcohol.

In other embodiments, the agents are modified by PEGylation, cholesterylation, or palmitoylation. The modification can be to any amino acid residue. In preferred embodiments, the modification is to the N-terminal amino acid of the agent, either directly to the N-terminal amino acid or by way coupling to the thiol group of a cysteine residue added to the N-terminus or a linker added to the N-terminus such as trimesoyl tris(3,5-dibromosalicylate (Ttds). In certain embodiments, the N-terminus of the agent comprises a cysteine residue to which a protecting group is coupled to the N-terminal amino group of the cysteine residue and the cysteine thiolate group is derivatized with N-ethylmaleimide, PEG group, cholesterol group, or palmitoyl group. In other embodiments, an acetylated cysteine residue is added to the N-terminus of the agents, and the thiol group of the cysteine is derivatized with N-ethylmaleimide, PEG group, cholesterol group, or palmitoyl group. In certain embodiments, the agent of the present invention is a conjugate. In certain embodiments, the agent of the present invention is a polypeptide consisting of an amino acid sequence which is bound with a methoxypolyethylene glycol(s) via a linker.

Substitutions of amino acids may be used to modify an agent of the present invention. The phrase “substitution of amino acids” as used herein encompasses substitution of amino acids that are the result of both conservative and non-conservative substitutions. Conservative substitutions are the replacement of an amino acid residue by another similar residue in a polypeptide. Typical, but not limiting, conservative substitutions are the replacements, for one another, among the aliphatic amino acids Ala, Val, Leu and Ile; interchange of Ser and Thr containing hydroxy residues, interchange of the acidic residues Asp and Glu, interchange between the amide-containing residues Asn and Gln, interchange of the basic residues Lys and Arg, interchange of the aromatic residues Phe and Tyr, and interchange of the small-sized amino acids Ala, Ser, Thr, Met, and Gly. Non-conservative substitutions are the replacement, in a polypeptide, of an amino acid residue by another residue which is not biologically similar. For example, the replacement of an amino acid residue with another residue that has a substantially different charge, a substantially different hydrophobicity, or a substantially different spatial configuration.

In certain embodiments, the present invention provides for one or more therapeutic agents. In certain embodiments, the one or more agents comprises a small molecule inhibitor, small molecule degrader (e.g., PROTAC), genetic modifying agent, antibody, antibody fragment, antibody-like protein scaffold, aptamer, protein, or any combination thereof.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In certain embodiments, the one or more agents is a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking a binding site or activating a receptor by binding to a ligand binding site).

One type of small molecule applicable to the present invention is a degrader molecule. Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57:107-123; and Lai et al., Modular PROTAC Design for the Degradation of Oncogenic BCR-ABL Angew Chem Int Ed Engl. 2016 Jan. 11; 55 (2): 807-810).

In certain embodiments, combinations of targets are modulated. In certain embodiments, an agent against one of the targets in a combination may already be known or used clinically. In certain embodiments, targeting the combination may require less of the agent as compared to the current standard of care and provide for less toxicity and improved treatment.

In certain embodiments, a method of treating PDAC comprises administering or more agents capable of modulating or maintaining (i.e., preventing a shift in) the expression, activity, or function of one or more biomarkers of a malignant signature, a CAF signature, an immune microniche signature, or a combination thereof. In certain embodiments, a method of treating PDAC comprises administering one or more agents capable of modulating or maintaining the expression, activity, or function of one or more biomarkers of a malignant signature such that the signature is shifted to a classical-like signature. In some embodiments, the method of treating PDAC comprises administering one or more agents capable of maintaining a classic-like malignant signature. Such signatures are described in greater detail elsewhere herein.

In some embodiments, the modulating agent is selected from HDAC inhibitor, a CDK4/6 inhibitor, a checkpoint inhibitor, an immunomodulator, an antibody, a genetic modulating agent, a chemotherapeutic, an antineoplastic agent, or a combination thereof.

In some embodiments, CD40 antibodies are used as a modulating agent alone or in combination with another agent or therapy such as a chemotherapy and/or PD-1 inhibition.

In some embodiments, a myeloid-specific immunomodulator (e.g., TGF-beta, losartan) can be used as modulating agent.

In some embodiments, the modulating agent can be an interferon (e.g., a Type I interferon).

In some embodiments, the modulating agent can be a BCL2 inhibitor.

In another aspect, embodiments disclosed herein provide a method of modulating a malignant signature comprising administering, to a population of cells comprising PDAC tumor cells, one or more agents capable of modulating the expression and/or activity of one or more biomarkers, expression programs, biologic programs, receptor-ligand interactions, cell state distribution, cell type distribution, or any combination thereof as in any of FIGS. 1B-1G, 2A-2D, 3A-3E, 4B-4D, 5A-5C, 6-13, 15-24, Tables 2, 3, 4, 5, 6, 7A-7B, or any combination thereof. In some embodiments, the population of cells include malignant cells and/or non-malignant cells.

In certain example embodiments, the modulating agent induces and/or suppresses expression and/or activity of one or more biomarkers, expression programs, biologic programs, receptor-ligand interactions, cell state distribution, cell type distribution, or any combination thereof as in any of FIGS. 1B-1G, 2A-2D, 3A-3E, 4B-4D, 5A-5C, 6-13, 15-24, Tables 2, 3, 4, 5, 6, 7A-7B, or any combination thereof.

### HDAC Inhibitor

In certain embodiments, the agent capable of modulating a signature as described herein is an HDAC inhibitor. Examples of HDAC inhibitors include hydroxamic acid derivatives, Short Chain Fatty Acids (SCFAs), cyclic tetrapeptides, benzamide derivatives, or electrophilic ketone derivatives, as defined herein. Specific non-limiting examples of HDAC inhibitors include: A) Hydroxamic acid derivatives selected from m-carboxycinnamic acid bishydroxamide (CBHA), Trichostatin A (TSA), Trichostatin C, Salicylhydroxamic Acid, Azelaic Bishydroxamic Acid (ABHA), Azelaic-1-Hydroxamate-9-Anilide (AAHA), 6-(3-Chlorophenylureido) carpoic Hydroxamic Acid (3Cl-UCHA), Oxamflatin, A-161906, Scriptaid, PXD-101, LAQ-824, CHAP, MW2796, and MW2996; B) Cyclic tetrapeptides selected from Trapoxin A, FR901228 (FK 228 or Depsipeptide), FR225497, Apicidin, CHAP, HC-Toxin, WF27082, and Chlamydocin; C) Short Chain Fatty Acids (SCFAs) selected from Sodium Butyrate, Isovalerate, Valerate, 4 Phenylbutyrate (4-PBA), Phenylbutyrate (PB), Propionate, Butyramide, Isobutyramide, Phenylacetate, 3-Bromopropionate, Tributyrin, Valproic Acid and Valproate; D)Benzamide Derivatives selected from C 1-994, MS-27-275 (MS-275) and a 3′-amino derivative of MS-27-275; E) Electrophilic Ketone Derivatives selected from a trifluoromethyl ketone and an α-keto amide such as an N-methyl-α-ketoamide; and F) Miscellaneous HDAC inhibitors including natural products, psammaplins and Depudecin.

Additional examples of HDAC inhibitors include vorinostat, romidepsin, chidamide, panobinostat, belinostat, mocetinostat, abexinostat, entinostat, resminostat, givinostat, quisinostat, CI-994, BML-210, M344, NVP-LAQ824, suberoylanilide hydroxamic acid (SAHA), MS-275, TSA, LAQ-824, trapoxin, depsipeptide, and tacedinaline.

Further examples of HDAC inhibitors include trichostatin A (TSA) ((R,2E,4E)-7-(4-(dimethylamino)phenyl)-N-hydroxy-4,6-dimethyl-7-oxohepta-2,4-dienamide); sulfonamides such as oxamflatin ((E)-N-hydroxy-5-(3-(phenylsulfonamido)phenyl) pent-2-en-4-ynamide). Other hydroxamic-acid-sulfonamide inhibitors of histone deacetylase are described in: Lavoie et al. (2001) Bioorg. Med. Chem. Lett. 11:2847-50; Bouchain et al. (2003) J. Med. Chem. 846:820-830; Bouchain et al. (2003) Curr. Med. Chem. 10:2359-2372; Marson et al. (2004) Bioorg. Med. Chem. Lett. 14:2477-2481; Finn et al. (2005) Helv. Chim. Acta 88:1630-1657; International Patent Publication Nos. WO 2002/030879, WO 2003/082288, WO 2005/0011661, WO 2005/108367, WO 2006123121, WO 2006/017214, WO 2006/017215, and US Patent Publication No. 2005/0234033. Other structural classes of histone deacetylase inhibitors include short chain fatty acids, cyclic peptides, and benzamides. Acharya et al. (2005) Mol. Pharmacol. 68:917-932.

Other examples of HDAC inhibitors include those disclosed in, e.g., Dokmanovic et al. (2007) Mol. Cancer. Res. 5:981; U.S. Pat. Nos. 7,642,275; 7,683,185; 7,732,475; 7,737,184; 7,741,494; 7,772,245; 7,795,304; 7,799,825; 7,803,800; 7,842,727; 7,842,835; U.S. Patent Publication No. 2010/0317739; U.S. Patent Publication No. 2010/0311794; U.S. Patent Publication No. 2010/0310500; U.S. Patent Publication No. 2010/0292320; and U.S. Patent Publication No. 2010/0291003.

### CDK 4 6 Inhibitor

In certain embodiments, the agent capable of modulating a signature as described herein is a cell cycle inhibitor (see e.g., Dickson and Schwartz, Development of cell-cycle inhibitors for cancer therapy, Curr Oncol. 2009 March; 16 (2): 36-43). In one embodiment, the agent capable of modulating a signature as described herein is a CDK4/6 inhibitor, such as LEE011, palbociclib (PD-0332991), and Abemaciclib (LY2835219) (see, e.g., U.S. Pat. No. 9,259,399B2; International Patent Publication No. WO 2016/025650A1; US Patent Publication No. 2014/0031325; US Patent Publication No. 2014/0080838; US Patent Publication No. 2013/0303543; US Patent Publication No. 2007/0027147; US Patent Publication No. 2003/0229026; US Patent Publication No 2004/0048915; US Patent Publication No. 2004/0006074; and US Patent Publication No. 2007/0179118, each of which is incorporated herein by reference in its entirety). Currently there are three CDK4/6 inhibitors that are either approved or in late-stage development: palbociclib (PD-0332991; Pfizer), ribociclib (LEE011; Novartis), and abemaciclib (LY2835219; Lilly) (see e.g., Hamilton and Infante, Targeting CDK4/6 in patients with cancer, Cancer Treatment Reviews, Volume 45, April 2016, Pages 129-138).

### Checkpoint Inhibitors

Because immune checkpoint inhibitors target the interactions between different cells in the tumor, their impact depends on multicellular circuits between malignant and non-malignant cells (Tirosh et al., 2016a). In principle, resistance can stem from different compartment of the tumor's ecosystem, for example, the proportion of different cell types (e.g., T cells, macrophages, fibroblasts), the intrinsic state of each cell (e.g., memory or dysfunctional T cell), and the impact of one cell on the proportions and states of other cells in the tumor (e.g., malignant cells inducing T cell dysfunction by expressing PD-L1 or promoting T cell memory formation by presenting neoantigens). These different facets are inter-connected through the cellular ecosystem: intrinsic cellular states control the expression of secreted factors and cell surface receptors that in turn affect the presence and state of other cells, and vice versa. In particular, brisk tumor infiltration with T cell has been associated with patient survival and improved immunotherapy responses (Fridman et al., 2012), but the determinants that dictate if a tumor will have high (“hot”) or low (“cold”) levels of T cell infiltration are only partially understood. Among multiple factors, malignant cells may play an important role in determining this phenotype (Spranger et al., 2015). Resolving this relationship with bulk genomics approaches has been challenging; single-cell RNA-seq (scRNA-seq) of tumors (Li et al., 2017; Patel et al., 2014; Tirosh et al., 2016a, 2016b; Venteicher et al., 2017) has the potential to shed light on a wide range of immune evasion mechanisms and immune suppression programs. In certain embodiments, a treatment may include inhibitors of HDAC and/or CDK4/6 and a checkpoint agonist. Immune checkpoint agonists may activate checkpoint signaling, for example, by binding to the checkpoint protein. The agonists may include a ligand (e.g., PD-L1). PD-1 agonist antibodies that mimic PD-1 ligand (PD-L1) have been described (see, e.g., US Patent Publication No. 2017/0088618A1; International Patent Publication No. WO 2018/053405 A1). Such agonist antibodies against any receptor described herein are applicable to the present invention.

### Antibodies

The term “antibody” is used interchangeably with the term “immunoglobulin” herein, and includes intact antibodies, fragments of antibodies, e.g., Fab, F (ab′) 2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, VHH and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight) of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g., the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, lgM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains. The “variable” domains of an antibody light chain are referred to interchangeably as “light chain variable regions”, “light chain variable domains”, “VL” regions or “VL” domains. The “variable” domains of an antibody heavy chain are referred to interchangeably as “heavy chain constant regions”, “heavy chain constant domains”, “VH” regions or “VH” domains.

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g., LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins-harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly cross react with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity, but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the CHI domain; (iii) the Fd fragment having V_{H} and C_{H}1 domains; (iv) the Fd′ fragment having V_{H} and C_{H}1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain polypeptides, form a pair of antigen binding regions (Zapata et al., Protein Eng. 8 (10): 1057-62 (1995); and U.S. Pat. No. 5,641,870).

As used herein, a “blocking” antibody or an antibody “antagonist” is one which inhibits or reduces biological activity of the antigen(s) it binds. For example, an antagonist antibody may bind an antigen or antigen receptor and inhibit the ability to suppress a response. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92 (6): 1981-1988 (1998); Chen et al., Cancer Res. 58 (16): 3668-3678 (1998); Harrop et al., J. Immunol. 161 (4): 1786-1794 (1998); Zhu et al., Cancer Res. 58 (15): 3209-3214 (1998); Yoon et al., J. Immunol. 160 (7): 3170-3179 (1998); Prat et al., J. Cell. Sci. III (Pt2): 237-247 (1998); Pitard et al., J. Immunol. Methods 205 (2): 177-190 (1997); Liautard et al., Cytokine 9 (4): 233-241 (1997); Carlson et al., J. Biol. Chem. 272 (17): 11295-11301 (1997); Taryman et al., Neuron 14 (4): 755-762 (1995); Muller et al., Structure 6 (9): 1153-1167 (1998); Bartunek et al., Cytokine 8 (1): 14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

The disclosure also encompasses nucleic acid molecules, in particular those that inhibit iHDAC and/or CDK4/6. Exemplary nucleic acid molecules include aptamers, siRNA, artificial microRNA, interfering RNA or RNAi, dsRNA, ribozymes, antisense oligonucleotides, and DNA expression cassettes encoding said nucleic acid molecules. Preferably, the nucleic acid molecule is an antisense oligonucleotide. Antisense oligonucleotides (ASO) generally inhibit their target by binding target mRNA and sterically blocking expression by obstructing the ribosome. ASOs can also inhibit their target by binding target mRNA thus forming a DNA-RNA hybrid that can be a substance for RNase H. Preferred ASOs include Locked Nucleic Acid (LNA), Peptide Nucleic Acid (PNA), and morpholinos Preferably, the nucleic acid molecule is an RNAi molecule, i.e., RNA interference molecule. Preferred RNAi molecules include siRNA, shRNA, and artificial miRNA. The design and production of siRNA molecules is well known to one of skill in the art (e.g., Hajeri P B, Singh S K. Drug Discov Today. 2009 14 (17-18): 851-8). The nucleic acid molecule inhibitors may be chemically synthesized and provided directly to cells of interest. The nucleic acid compound may be provided to a cell as part of a gene delivery vehicle. Such a vehicle is preferably a liposome or a viral gene delivery vehicle.

### Genetic Modifying Agents

In certain embodiments, the one or more modulating agents may be a genetic modifying agent. In certain embodiments, the one or more modulating agents may be a genetic modifying agent. The genetic modifying agent may comprise a CRISPR system, a zinc finger nuclease system, a TALEN, a meganuclease or RNAi system. In some embodiments, a polynucleotide of the present invention described elsewhere herein can be modified using a genetic modifying agent (e.g., one or more genes as in any of FIGS. 1B-1G, 2A-2D, 3A-3E, 4B-4D, 5A-5C, 6-13, 15-24, Tables 2, 3, 4, 5, 6, 7A-7B, or any combination thereof).

### CRISPR-Cas Modification

In some embodiments, a polynucleotide of the present invention described elsewhere herein can be modified using a CRISPR-Cas and/or Cas-based system.

In general, a CRISPR-Cas or CRISPR system as used in herein and in documents, such as International Patent Publication No. WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two class are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

### Class 1 CRISPR-Cas Systems

In some embodiments, the CRISPR-Cas system that can be used to modify a polynucleotide of the present invention described herein can be a Class 1 CRISPR-Cas system. Class 1 CRISPR-Cas systems are divided into types I, II, and IV. Makarova et al. 2020. Nat. Rev. 18:67-83., particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-C, III-D, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes (IV-A, IV-B, and IV-C). Makarova et al., 2020. Class 1 systems also include CRISPR-Cas variants, including Type I-A, I-B, I-E, I—F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas. 1709035114; see also, Makarova et al. 2018. The CRISPR Journal, v. 1, n5, FIG. 5.

The Class 1 systems typically comprise a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

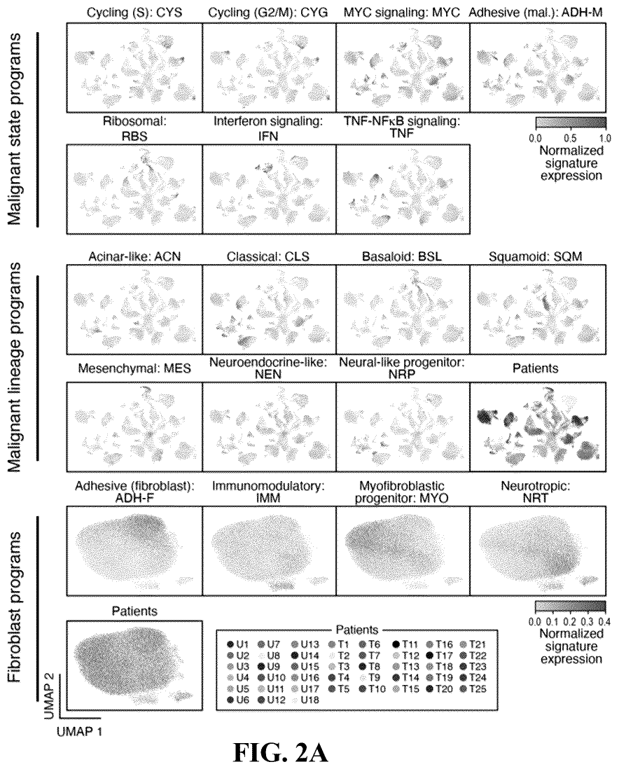

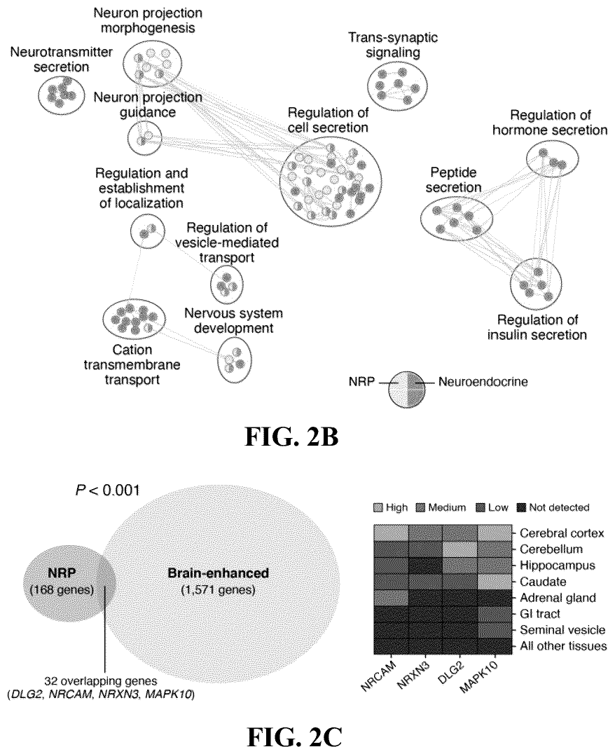

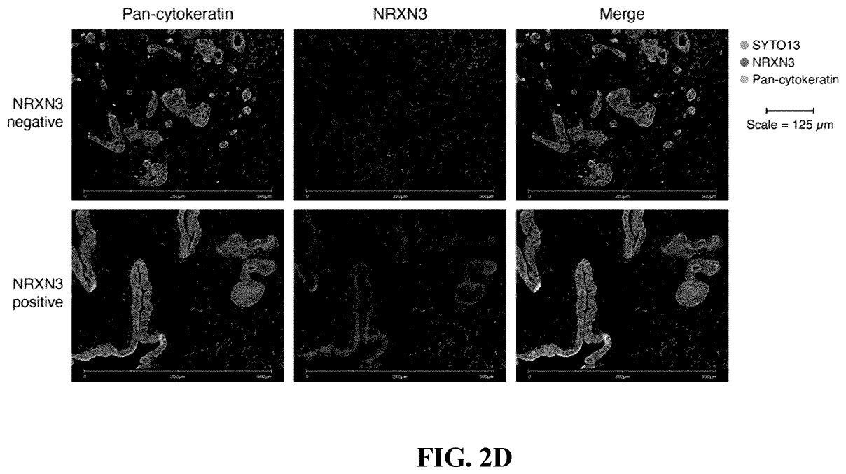

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374:20180087, DOI: 10.1098/rstb.2018.0087 and Makarova et al. 2020.

Class 1 CRISPR-Cas system effector complexes can, in some embodiments, include a small subunit (for example, Cas11). See, e.g., FIGS. 1 and 2. Koonin E V, Makarova K S. 2019 Origins and Evolution of CRISPR-Cas systems. Phil. Trans. R. Soc. B 374:20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cas6, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1 (V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-FI CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-UI CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2cl), Cas12c (C2c3), Cas12d (CasY), Cas12e (CasX), and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g., VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154 (6): 1380-1389), Cas12 (Liu et al. Nature Communications, 8, 2095 (2017), and Cas13 (International Patent Publication Nos. WO 2019/005884 and WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33 (2): 139-142 and International Patent Publication WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein is attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C·G base pair into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). Rees and Liu. 2018. Nat. Rev. Genet. 19 (12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19 (12): 770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas, can have nickase functionality, and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

In certain example embodiments, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA base editor may be used to delete or introduce a post-translation modification site in the expressed mRNA. In contrast to DNA base editors, whose edits are permanent in the modified cell, RNA base editors can provide edits where finer, temporal control may be needed, for example in modulating a particular immune response. Example Type VIRNA-base editing systems are described in Cox et al. 2017. Science 358:1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstituble halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system. See e.g., Anzalone et al. 2019. Nature. 576:149-157. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion and combinations thereof. Generally, a prime editing system, as exemplified by PEI, PE2, and PE3 (Id.), can include a reverse transcriptase fused or otherwise coupled or associated with an RNA-programmable nickase and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576:149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g., is a Cas9 nickase). In some embodiments, the Cas polypeptide is fused to the reverse transcriptase. In some embodiments, the Cas polypeptide is linked to the reverse transcriptase.

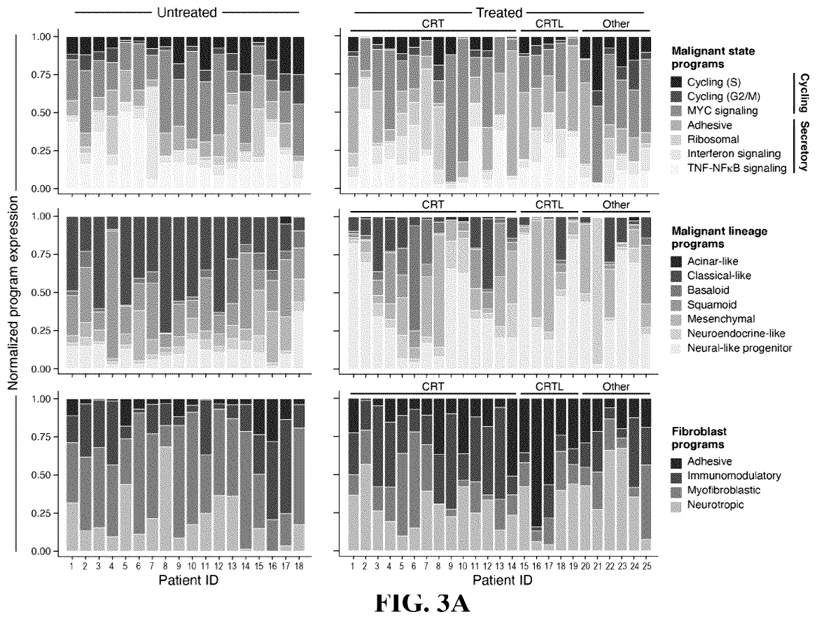

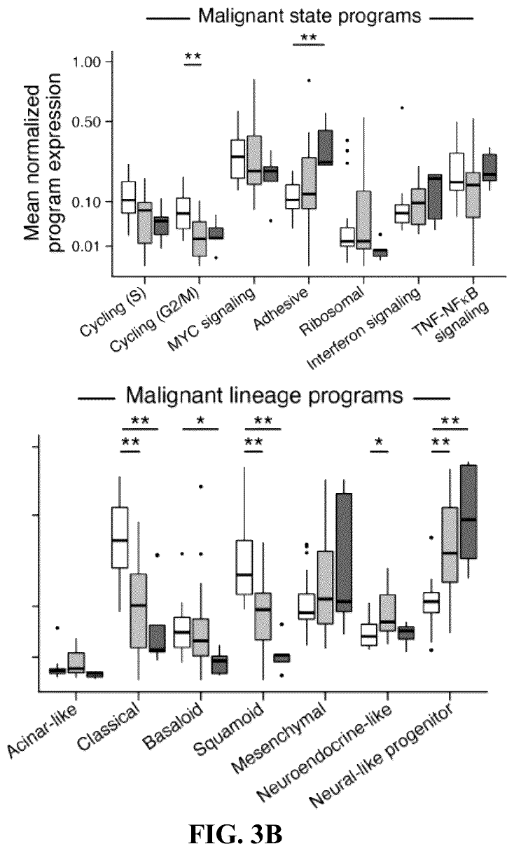

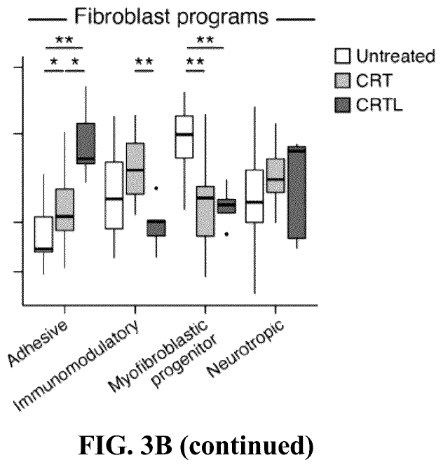

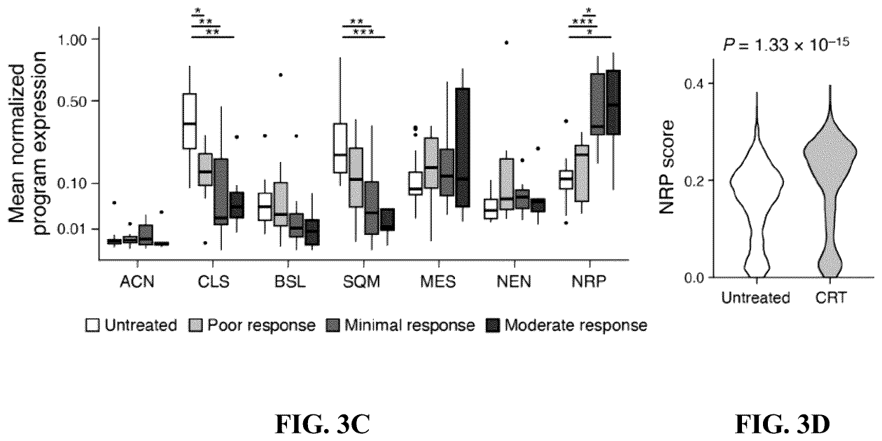

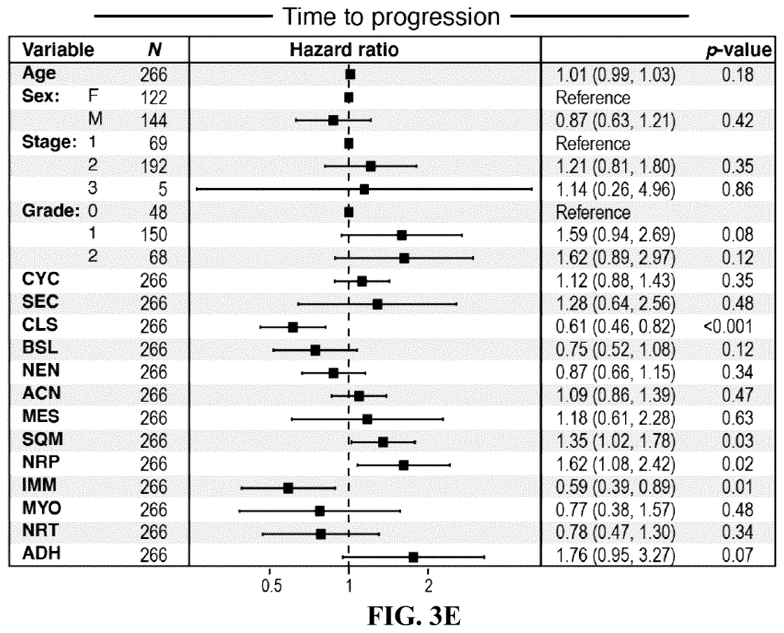

In some embodiments, the prime editing system can be a PE1 system or variant thereof, a PE2 system or variant thereof, or a PE3 (e.g., PE3, PE3b) system. See e.g., Anzalone et al. 2019. Nature. 576:149-157, particularly at pgs. 2-3, FIGS. 2a, 3a-3f, 4a-4b, Extended data FIGS. 3a-3b, 4.

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576:149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST systems can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi: 10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### Guide Molecules

The CRISPR-Cas or Cas-Based system described herein can, in some embodiments, include one or more guide molecules. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36 (4) 702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106 (1): 23-24; and PA Carr and GM Church, 2009, Nature Biotechnology 27 (12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16 (4): 504-517. Table 1 (from Gleditzsch et al. 2019) below shows several Cas polypeptides and the PAM sequence they recognize.

TABLE 1
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including LbCpf1 | TTTV |
| and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein His A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523 (7561): 481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155 (Pt. 3): 733-740; Atschul et al. 1990. J. Mol. Biol. 215:403-410; Biswass et al. 2013 RNA Biol. 10:817-827; and Grissa et al. 2007. Nucleic Acid Res. 35: W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016.Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16 (4): 504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16 (4): 504-517.

Overall Type VI CRISPR-Cas systems appear to have less restrictive rules for substrate (e.g., target sequence) recognition than those that target DNA (e.g., Type V and type II). Sequences related to nucleus targeting and transportation

In some embodiments, one or more components (e.g., the Cas protein and/or deaminase) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO:3) or PKKKRKVEAS (SEQ ID NO:4); the NLS from nucleoplasmin (e.g., the nucleoplasmin bipartite NLS with the sequence KRPAATKKAGQAKKKK (SEQ ID NO:5)); the c-myc NLS having the amino acid sequence PAAKRVKLD (SEQ ID NO:6) or RQRRNELKRSP (SEQ ID NO:7); the hRNPA1 M9 NLS having the sequence NQSSNFGPMKGGNFGGRSSGPYGGGGQYFAKPRNQGGY (SEQ ID NO:8); the sequence RMRIZFKNKGKDTAELRRRRVEVSVELRKAKKDEQILKRRNV (SEQ ID NO:9) of the IBB domain from importin-alpha; the sequences VSRKRPRP (SEQ ID NO: 10) and PPKKARED (SEQ ID NO: 11) of the myoma T protein; the sequence PQPKKKPL (SEQ ID NO:12) of human p53; the sequence SALIKKKKKMAP (SEQ ID NO:13) of mouse c-abl IV; the sequences DRLRR (SEQ ID NO:14) and PKQKKRK (SEQ ID NO:15) of the influenza virus NS1; the sequence RKLKKKIKKL (SEQ ID NO:16) of the Hepatitis virus delta antigen; the sequence REKKKFLKRR (SEQ ID NO:17) of the mouse Mx1 protein; the sequence KRKGDEVDGVDEVAKKKSKK (SEQ ID NO:18) of the human poly(ADP-ribose) polymerase; and the sequence RKCLQAGMNLEARKTKK (SEQ ID NO: 19) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g. aptamers) for adapter proteins, which may be linked to or fused to a nucleotide deaminase or catalytic domain thereof. When such a guide forms a CRISPR complex (e.g., CRISPR-Cas protein binding to guide and target), the adapter proteins bind and the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g., due to steric hindrance within the three-dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Templates

In some embodiments, a composition for engineering cells comprises a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include a sequence that corresponds to both, a first site on the target sequence that is cleaved in a first Cas protein mediated event, and a second site on the target sequence that is cleaved in a second Cas protein mediated event.

In certain embodiments, the template nucleic acid can include a sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include a sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include a sequence which, when integrated, results in decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include a sequence which results in a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 180+/−10, 190+/−10, 200+/−10, 210+/−10, or 220+/−10 nucleotides in length. In an embodiment, the template nucleic acid may be 30+/−20, 40+/−20, 50+/−20, 60+/−20, 70+/−20, 80+/−20, 90+/−20, 100+/−20, 1 10+/−20, 120+/−20, 130+/−20, 140+/−20, 150+/−20, 160+/−20, 170+/−20, 180+/−20, 190+/−20, 200+/−20, 210+/−20, or 220+/−20 nucleotides in length. In an embodiment, the template nucleic acid is 10 to 1,000, 20 to 900, 30 to 800, 40 to 700, 50 to 600, 50 to 500, 50 to 400, 50 to300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

### Zinc Finger Nucleases

In some embodiments, the polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ Or 34 Or 35, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ Of 34 Or 35) z, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 20)
M D P I R S R T P S P A R E L L S G P Q P D G V Q P T A D R G V S P P A G G P
L D G L P A R R T M S R T R L P S P P A P S P A F S A D S F S D L L R Q F D P S L F N T S
L F D S L P P F G A H H T E A A T G E W D E V Q S G L R A A D A P P P T M R V A V T A
A R P P R A K P A P R R R A A Q P S D A S P A A Q V D L R T L G Y S Q Q Q Q E K I K P
K V R S T V A Q H H E A L V G H G F T H A H I V A L S Q H P A A L G T V A V K Y Q D
M I A A L P E A T H E A I V G V G K Q W S G A R A L E A L L T V A G E L R G P P L Q L
D T G Q L L K I A K R G G V T A V E A V H A W R N A L T G A P L N

An exemplary amino acid sequence of a C-terminal capping region is:

(SEQ ID NO: 21)
R P A L E S I V A Q L S R P D P A L A A L T N D H L V A L A C L G G R P A L
D A V K K G L P H A P A L I K R T N R R I P E R T S H R V A D H A Q V V R V L G F F Q
C H S H P A Q A F D D A M T Q F G M S R H G L L Q L F R R V G V T E L E A R S G T L P
P A S Q R W D R I L Q A S G M K R A K P S P T S T Q T P D Q A S L H A F A D S L E R D
L D A P S P M H E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments, the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### RNAi

In certain embodiments, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e., although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated herein by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Combination Therapies

Described herein are combination therapies that can be used in a subject in need thereof having PDAC. In some embodiments, the combination therapy can include detection and and/or monitoring a PDAC tumor signature described elsewhere herein. In some embodiments, the combination therapy can include neoadjuvant treatment, PDAC tumor resection, administration of a PDAC signature modulating agent, a post neoadjuvant therapy, or a combination thereof.

### Phased Combination Therapy

In certain embodiments, a subject in need thereof is treated with a combination therapy, which may be a phased combination therapy. Phased combination therapies are combination therapies are those that contain various treatment phases where each phase can incorporate a different therapy approach. In some embodiments, the initiation of each phase can be dictated by achieving a particular milestone, such as a specific signature, subject response, time, number of doses, or other predetermined standard.

In some embodiments, the phased combination therapy can include administration of one or more PDAC modulators as described elsewhere herein, PDAC tumor resection, neoadjuvant administration, or a combination thereof. In some embodiments, the phased combination therapy can include detecting and/or monitoring a PDAC signature described in greater detail elsewhere herein.

In some embodiments, phased combination therapy may be a treatment regimen comprising checkpoint inhibition followed by a CDK4/6 inhibitor, an HDAC inhibitor, an/or checkpoint inhibitor combination. Checkpoint inhibitors may be administered at regular intervals, for example, daily, weekly, every two weeks, every month. The combination therapy may be administered when a signature disclosed herein is detected. This may be after two weeks to six months after the initial checkpoint inhibition. The immunotherapy may be adoptive cell transfer therapy, as described herein or may be an inhibitor of any check point protein described herein. The checkpoint blockade therapy may comprise anti-TIM3, anti-CTLA4, anti-PD-L1, anti-PD1, anti-TIGIT, anti-LAG3, or combinations thereof. Specific check point inhibitors include, but are not limited to, anti-CTLA4 antibodies (e.g., Ipilimumab), anti-PD-1 antibodies (e.g., Nivolumab, Pembrolizumab), and anti-PD-L1 antibodies (e.g., Atezolizumab). Dosages for the immunotherapy and/or CDK4/6 inhibitors may be determined according to the standard of care for each therapy and may be incorporated into the standard of care (see, e.g., Rivalland et al., Standard of care in immunotherapy trials: Challenges and considerations, Hum Vaccin Immunother. 2017 July; 13 (9): 2164-2178; and Pernas et al., CDK4/6 inhibition in breast cancer: current practice and future directions, Ther Adv Med Oncol. 2018). The standard of care is the current treatment that is accepted by medical experts as a proper treatment for a certain type of disease and that is widely used by healthcare professionals. Standard or care is also called best practice, standard medical care, and standard therapy.

### Pharmaceutical Formulations and Administration

### Administration

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, PA (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32 (2): 210-8 (2000), Wang W. “Lyophilization and development of solid protein pharmaceuticals.” Int. J. Pharm. 203 (1-2): 1-60 (2000), Charman WN “Lipids, lipophilic drugs, and oral drug delivery-some emerging concepts.” J Pharm Sci. 89 (8): 967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The medicaments of the invention are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Administration of medicaments of the invention may be by any suitable means that results in a compound concentration that is effective for treating or inhibiting (e.g., by delaying) the development of a disease. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

The agents disclosed herein may be used in a pharmaceutical composition when combined with a pharmaceutically acceptable carrier. Such compositions comprise a therapeutically-effective amount of the agent and a pharmaceutically acceptable carrier. Such a composition may also further comprise (in addition to an agent and a carrier) diluents, fillers, salts, buffers, stabilizers, solubilizers, and other materials well known in the art. Compositions comprising the agent can be administered in the form of salts provided the salts are pharmaceutically acceptable. Salts may be prepared using standard procedures known to those skilled in the art of synthetic organic chemistry.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to specific agents (e.g., neuromedin U receptor agonists or antagonists), also include the pharmaceutically acceptable salts thereof.

Methods of administrating the pharmacological compositions, including agonists, antagonists, antibodies or fragments thereof, to an individual include, but are not limited to, intradermal, intrathecal, intramuscular, intraperitoneal, intravenous, subcutaneous, intranasal, epidural, by inhalation, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

Various delivery systems are known and can be used to administer the pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to, a delivery pump (See, for example, Saudek, et al., New Engl. J. Med. 321:574 (1989) and a semi-permeable polymeric material (See, for example, Howard, et al., J. Neurosurg. 71:105 (1989)). Additionally, the controlled release system can be placed in proximity of the therapeutic target (e.g., a tumor), thus requiring only a fraction of the systemic dose. See, for example, Goodson, In: Medical Applications of Controlled Release, 1984. (CRC Press, Boca Raton, Fla.).

The amount of the agents which will be effective in the treatment of a particular disorder or condition will depend on the nature of the disorder or condition, and may be determined by standard clinical techniques by those of skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of the agent with which to treat each individual patient. In certain embodiments, the attending physician will administer low doses of the agent and observe the patient's response. Larger doses of the agent may be administered until the optimal therapeutic effect is obtained for the patient, and at that point the dosage is not increased further. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases. In certain embodiments, suitable dosage ranges for intravenous administration of the agent are generally about 5-500 micrograms (ug) of active compound per kilogram (Kg) body weight. Suitable dosage ranges for intranasal administration are generally about 0.01 pg/kg body weight to 1 mg/kg body weight. In certain embodiments, a composition containing an agent of the present invention is subcutaneously injected in adult patients with dose ranges of approximately 5 to 5000 ug/human and preferably approximately 5 to 500 ug/human as a single dose. It is desirable to administer this dosage 1 to 3 times daily. Effective doses may be extrapolated from dose-response curves derived from in vitro or animal model test systems. Suppositories generally contain active ingredient in the range of 0.5% to 10% by weight; oral formulations preferably contain 10% to 95% active ingredient. Ultimately the attending physician will decide on the appropriate duration of therapy using compositions of the present invention. Dosage will also vary according to the age, weight and response of the individual patient.

Methods for administering antibodies for therapeutic use is well known to one skilled in the art. In certain embodiments, small particle aerosols of antibodies or fragments thereof may be administered (see e.g., Piazza et al., J. Infect. Dis., Vol. 166, pp. 1422-1424, 1992; and Brown, Aerosol Science and Technology, Vol. 24, pp. 45-56, 1996). In certain embodiments, antibodies are administered in metered-dose propellant driven aerosols. In preferred embodiments, antibodies are used as agonists to depress inflammatory diseases or allergen-induced asthmatic responses. In certain embodiments, antibodies may be administered in liposomes, i.e., immunoliposomes (see, e.g., Maruyama et al., Biochim. Biophys. Acta, Vol. 1234, pp. 74-80, 1995). In certain embodiments, immunoconjugates, immunoliposomes or immunomicrospheres containing an agent of the present invention is administered by inhalation.

In certain embodiments, antibodies may be topically administered to mucosa, such as the oropharynx, nasal cavity, respiratory tract, gastrointestinal tract, eye such as the conjunctival mucosa, vagina, urogenital mucosa, or for dermal application. In certain embodiments, antibodies are administered to the nasal, bronchial or pulmonary mucosa. In order to obtain optimal delivery of the antibodies to the pulmonary cavity in particular, it may be advantageous to add a surfactant such as a phosphoglyceride, e.g., phosphatidylcholine, and/or a hydrophilic or hydrophobic complex of a positively or negatively charged excipient and a charged antibody of the opposite charge.

Other excipients suitable for pharmaceutical compositions intended for delivery of antibodies to the respiratory tract mucosa may be a) carbohydrates, e.g., monosaccharides such as fructose, galactose, glucose. D-mannose, sorbiose, and the like; disaccharides, such as lactose, trehalose, cellobiose, and the like; cyclodextrins, such as 2-hydroxypropyl-β-cyclodextrin; and polysaccharides, such as raffinose, maltodextrins, dextrans, and the like; b) amino acids, such as glycine, arginine, aspartic acid, glutamic acid, cysteine, lysine and the like; c) organic salts prepared from organic acids and bases, such as sodium citrate, sodium ascorbate, magnesium gluconate, sodium gluconate, tromethamine hydrochloride, and the like: d) peptides and proteins, such as aspartame, human serum albumin, gelatin, and the like; e) alditols, such mannitol, xylitol, and the like, and f) polycationic polymers, such as chitosan or a chitosan salt or derivative.

For dermal application, the antibodies of the present invention may suitably be formulated with one or more of the following excipients: solvents, buffering agents, preservatives, humectants, chelating agents, antioxidants, stabilizers, emulsifying agents, suspending agents, gel-forming agents, ointment bases, penetration enhancers, and skin protective agents.

Examples of solvents are e.g. water, alcohols, vegetable or marine oils (e.g. edible oils like almond oil, castor oil, cacao butter, coconut oil, corn oil, cottonseed oil, linseed oil, olive oil, palm oil, peanut oil, poppy seed oil, rapeseed oil, sesame oil, soybean oil, sunflower oil, and tea seed oil), mineral oils, fatty oils, liquid paraffin, polyethylene glycols, propylene glycols, glycerol, liquid polyalkylsiloxanes, and mixtures thereof.

Examples of buffering agents are, e.g., citric acid, acetic acid, tartaric acid, lactic acid, hydrogenphosphoric acid, diethyl amine etc. Suitable examples of preservatives for use in compositions are parabenes, such as methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol, or mixtures of preservatives.

Examples of humectants are glycerin, propylene glycol, sorbitol, lactic acid, urea, and mixtures thereof.

Examples of antioxidants are butylated hydroxy anisole (BHA), ascorbic acid and derivatives thereof, tocopherol and derivatives thereof, cysteine, and mixtures thereof.

Examples of emulsifying agents are naturally occurring gums, e.g., gum acacia or gum tragacanth; naturally occurring phosphatides, e.g., soybean lecithin, sorbitan monooleate derivatives: wool fats; wool alcohols; sorbitan esters; monoglycerides; fatty alcohols; fatty acid esters (e.g., triglycerides of fatty acids); and mixtures thereof.

Examples of suspending agents are e.g., celluloses and cellulose derivatives such as, e.g., carboxymethyl cellulose, hydroxyethylcellulose, hydroxypropylcellulose, hydroxypropylmethylcellulose, carraghenan, acacia gum, arabic gum, tragacanth, and mixtures thereof.

Examples of gel bases, viscosity-increasing agents or components which are able to take up exudate from a wound are: liquid paraffin, polyethylene, fatty oils, colloidal silica or aluminum, zinc soaps, glycerol, propylene glycol, tragacanth, carboxyvinyl polymers, magnesium-aluminum silicates, Carbopol®, hydrophilic polymers such as, e.g. starch or cellulose derivatives such as, e.g., carboxymethylcellulose, hydroxyethylcellulose and other cellulose derivatives, water-swellable hydrocolloids, carragenans, hyaluronates (e.g., hyaluronate gel optionally containing sodium chloride), and alginates including propylene glycol alginate.

Examples of ointment bases are e.g., beeswax, paraffin, cetanol, cetyl palmitate, vegetable oils, sorbitan esters of fatty acids (Span), polyethylene glycols, and condensation products between sorbitan esters of fatty acids and ethylene oxide, e.g., polyoxyethylene sorbitan monooleate (Tween).

Examples of hydrophobic or water-emulsifying ointment bases are paraffins, vegetable oils, animal fats, synthetic glycerides, waxes, lanolin, and liquid polyalkylsiloxanes. Examples of hydrophilic ointment bases are solid macrogols (polyethylene glycols). Other examples of ointment bases are triethanolamine soaps, sulphated fatty alcohol and polysorbates.

Examples of other excipients are polymers such as carmelose, sodium carmelose, hydroxypropylmethylcellulose, hydroxyethylcellulose, hydroxypropylcellulose, pectin, xanthan gum, locust bean gum, acacia gum, gelatin, carbomer, emulsifiers like vitamin E, glyceryl stearates, cetanyl glucoside, collagen, carrageenan, hyaluronates and alginates and chitosans.

The dose of antibody required in humans to be effective in the treatment or prevention of allergic inflammation differs with the type and severity of the allergic condition to be treated, the type of allergen, the age and condition of the patient, etc. Typical doses of antibody to be administered are in the range of 1 μg to 1 g, preferably 1-1000 μg, more preferably 2-500, even more preferably 5-50, most preferably 10-20 μg per unit dosage form. In certain embodiments, infusion of antibodies of the present invention may range from 10-500 mg/m2.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection.

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes, hours, days, months, years or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Pharmaceutical Formulations

Also described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. In some embodiments, the pharmaceutical formulation can include, such as an active ingredient, a PDAC signature modulating agent or other PDAC treatment or agent described in greater detail elsewhere herein.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a PDAC, neoadjuvant resistant malignant PDAC cells, and/or a symptom thereof. As used herein, “agent” generally refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g., polynucleotides, amino acids, peptides, polypeptides, antibodies, aptamers, ribozymes, hormones, immunomodulators, antipyretics, anxiolytics, antipsychotics, analgesics, antispasmodics, anti-inflammatoir anti-histamines, anti-infectives, chemotherapeutics, and combinations thereof.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects. In some embodiments, the one or more therapeutic effects are to treat PDAC or symptom thereof, to modulate or maintain a PDAC tumor signature, or a combination thereof.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, ug, mg, or g or be any numerical value or subrange within any of these ranges.

In some embodiments, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value or subrange within any of these ranges.

In other embodiments, the effective amount, least effective amount, and/or therapeutically effective amount of the primary and optional secondary active agent be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value or subrange within any of these ranges.

In some embodiments, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In some embodiments where a cell or cell population is present in the pharmaceutical formulation (e.g., as a primary and/or or secondary active agent), the effective amount of cells can be any amount ranging from about 1 or 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL or any numerical value or subrange within any of these ranges.

In some embodiments, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In some embodiments, the effective amount can be about 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L or any numerical value or subrange within these ranges. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more or any numerical value or subrange within these ranges.

In some embodiments, the amount or effective amount of the one or more of the active agent(s) described herein contained in the pharmaceutical formulation can range from about 1 μg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total secondary active agent in the pharmaceutical formulation. In additional embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington-The science and practice of pharmacy”, 20th ed., Lippincott Williams & Wlkins, Baltimore, MD, 2000, and “Pharmaceutical dosage forms and drug delivery systems”, 6th Edition, Ansel et al., (Media, PA: Wlliams and Wlkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

Methods of Screening for Pdac Treatments and/or Preventives

Described in certain example embodiments herein is a method of screening for one or more agents capable of treating or preventing PDAC or progression thereof. Described in certain example embodiments herein is a method of screening for one or more agents capable of treating or preventing PDAC or progression thereof comprising (a) contacting a PDAC tumor cell or cell population or an organoid or organoid cell population derived therefrom with a test agent or library of test agents, wherein the PDAC tumor cells or organoid cells have an initial cell state, expression signature, and/or expression program; (b) determining a fraction of PDAC or organoid cells having a desired cell state, expression signature, and/or expression program and/or determining a fraction of PDAC or organoid cells having an undesired cell state, expression signature, and/or expression program; and (c) selecting test agents that shift the initial PDAC or organoid cell state, expression signature, and/or expression program to a desired cell state, expression signature, and/or expression program and/or prevent a shift in the initial PDAC or organoid cell state, expression signature, and/or expression program to an undesired cell state, expression signature, and/or expression program or away from a desired cell state, expression signature, and/or expression program such that the fraction of PDAC and/or organoid cells having the desired cell state, expression signatures, and/or expression program is above a set cutoff limit.

As used herein, the term “organoid” refers to a cell cluster or aggregate that resembles an organ, or part of an organ, and possesses cell types relevant to that particular organ. Organoid systems have been described previously, for example, for brain, retinal, stomach, lung, thyroid, small intestine, colon, liver, kidney, pancreas, prostate, mammary gland, fallopian tube, taste buds, salivary glands, and esophagus (see, e.g., Clevers, Modeling Development and Disease with

Organoids, Cell. 2016 Jun. 16; 165 (7): 1586-1597). They also have been developed for cancers, including those to model patient specific tumors. See e.g., LeSavage et al. Nat. Mat. 21, pages 143-159 (2022); Drost and Clevers. Nat. Rev. Canc. 18, pages 407-418 (2018); Verduin et al. Front. Oncol. 18 Mar. 2021. https://doi.org/10.3389/fonc.2021.641980; Veninga and Voest. Cancer Cell. Volume 39, Issue 9, 13 Sep. 2021, Pages 1190-1201; Nagle et al., Seminars in Cancer Biol. Volume 53, December 2018, Pages 258-264; Xu et al., J Hematol Oncol. 11, Article number: 116 (2018); Low et al., Nat. Cancer. 2020 August; 1 (8): 761-773; and Grönholm, et al. Cancer Res. 2021. Volume 81, Issue 12. DOI: 10.1158/0008-5472.CAN-20-402, which are incorporated by reference as if expressed in their entireties herein and can be adapted for use with the present invention. In some embodiments, herein the organoid is a PDAC organoid or otherwise an organoid derived from PDAC tumor cells or tissue. In some embodiments, the organoid is a patient specific organoid.

In certain example embodiments, the desired PDAC or organoid cell state, expression signature, and/or expression program is a PDAC malignant cell classical-like program or a CAF immunomodulatory program.

In certain example embodiments, the undesired PDAC or organoid cell state, expression signature, and/or expression program is a PDAC malignant cell neural-like progenitor program, a PDAC malignant cell neuroendocrine-like program, a PDAC malignant cell squamoid program, a PDAC malignant cell basaloid program, a PDAC malignant cell mesenchymal program, or a CAF adhesive program.

In certain example embodiments, the initial cell state, expression signature, and/or expression program of the PDAC cell or cell population and/or the organoid or organoid cells is a PDAC malignant cell neural-like progenitor program.

In certain example embodiments, the PDAC tumor cell or cells are obtained from a subject in need thereof to be treated.

In certain example embodiments, the subject has had or is concurrently receiving a PDAC neoadjuvant therapy.

In certain example embodiments, the neural-like progenitor program comprises one or more drug efflux programs and/or genes, apoptosis regulation programs and/or genes, chemoresistance programs and/or genes, tumor-nerve cross-talk programs and/or genes, neuronal gene expression programs, or neuronal development/migration/adhesion programs and/or genes, tissue stem cell module programs and/or genes, organ morphogenesis programs and/or genes, or hepatocyte nuclear factor activity programs and/or genes.

The methods of nucleic acid and/or protein analysis described in greater detail elsewhere herein (see e.g., section on methods of diagnosing, prognosing and/or treating PDAC) can be utilized for evaluating environmental stress and/or state, for screening of chemical and/or biologic libraries, and to screen or identify structural, syntenic, genomic, and/or organism and species variations. Aspects of the present disclosure relate to the correlation of an environmental stress or state with the spatial proximity and/or epigenetic profile of the nucleic acids in a sample of cells, for example a culture of cells, can be exposed to an environmental stress, such as but not limited to heat shock, osmolarity, hypoxia, cold, oxidative stress, radiation, starvation, a chemical or biologic (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value.

In some embodiments, the disclosed methods can be used to screen chemical and/or biologic libraries for agents that modulate chromatin architecture epigenetic profiles, and/or relationships thereof. By exposing cells, or fractions thereof, tissues, or even whole animals, to different members of the chemical libraries, and performing the methods described herein, different members of a chemical library can be screened for their effect on architecture epigenetic profiles, and/or relationships thereof simultaneously in a relatively short amount of time, for example using a high throughput method.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

A further aspect of the invention relates to a method for identifying an agent capable of modulating one or more phenotypic aspects of a cell or cell population as disclosed herein, comprising: a) applying a candidate agent to the cell or cell population; b) detecting modulation of one or more phenotypic aspects of the cell or cell population by the candidate agent, thereby identifying the agent. The phenotypic aspects of the cell or cell population that is modulated may be a gene signature or biological program specific to a cell type or cell phenotype or phenotype specific to a population of cells (e.g., an inflammatory phenotype or suppressive immune phenotype). In certain embodiments, steps can include administering candidate modulating agents to cells, detecting identified cell (sub) populations for changes in signatures, or identifying relative changes in cell (sub) populations which may comprise detecting relative abundance of particular gene signatures.

The term “modulate” broadly denotes a qualitative and/or quantitative alteration, change or variation in that which is being modulated. Where modulation can be assessed quantitatively-for example, where modulation comprises or consists of a change in a quantifiable variable such as a quantifiable property of a cell or where a quantifiable variable provides a suitable surrogate for the modulation-modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of an immune cell or immune cell population may be modulated without substantially altering other (unintended, undesired) phenotypic aspect(s).

The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

The methods of phenotypic analysis can be utilized for evaluating environmental stress and/or state, for screening of chemical libraries, and to screen or identify structural, syntenic, genomic, and/or organism and species variations. For example, a culture of cells, can be exposed to an environmental stress, such as but not limited to heat shock, osmolarity, hypoxia, cold, oxidative stress, radiation, starvation, a chemical (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value. By exposing cells, or fractions thereof, tissues, or even whole animals, to different members of the chemical libraries, and performing the methods described herein, different members of a chemical library can be screened for their effect on immune phenotypes thereof simultaneously in a relatively short amount of time, for example using a high throughput method.

Aspects of the present disclosure relate to the correlation of an agent with the spatial proximity and/or epigenetic profile of the nucleic acids in a sample of cells. In some embodiments, the disclosed methods can be used to screen chemical libraries for agents that modulate chromatin architecture epigenetic profiles, and/or relationships thereof.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures or biological programs of the present invention may be used to screen for drugs that reduce the signature or biological program in cells as described herein. The signature or biological program may be used for GE-HTS. In certain embodiments, pharmacological screens may be used to identify drugs that are selectively toxic to cells having a signature.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to screen for small molecules capable of modulating a signature or biological program of the present invention in silico.

### KITS

Any of the compounds, compositions, formulations, particles, cells, devices, or any combination thereof described herein, or a combination thereof can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agents) contained in the kit are administered simultaneously, the combination kit can contain the active agents in a single formulation, such as a pharmaceutical formulation, (e.g., a tablet) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, cells, described herein or a combination thereof contained therein, safety information regarding the content of the compounds, compositions, formulations (e.g., pharmaceutical formulations), particles, and cells described herein or a combination thereof contained therein, information regarding the dosages, indications for use, and/or recommended treatment regimen(s) for the compound(s) and/or pharmaceutical formulations contained therein. In some embodiments, the instructions can provide directions for administering the compounds, compositions, formulations, particles, and cells described herein or a combination thereof to a subject in need thereof.

In some embodiments, the subject in need thereof is in need of a treatment or prevention for a pancreatic disease or a symptom thereof. In some embodiments, the pancreatic disease can be a pancreatic cancer. In some embodiments, the pancreatic disease is PDAC. In some embodiments, the instructions provide that the subject in need thereof or a tissue and/or cell(s) from said subject, to which the compounds, compositions, formulations, particles, cells, described herein or a combination thereof can be administered, has one or more PDAC signatures described herein. In some embodiments, the instructions and/or a label includes diagnostic, prognostic and/or PDAC treatment guidance based on one or more detected PDAC signatures described herein.

Further embodiments are illustrated in the following Examples which are given for illustrative purposes only and are not intended to limit the scope of the invention.

### EXAMPLES

### Example 1—Single-Nucleus and Spatial Whole Transcriptome Profiling of Pancreatic Cancer Reveals Multicellular Communities and Enrichment of a Neural-Like Progenitor Phenotype after Neoadjuvant Treatment

Pancreatic ductal adenocarcinoma (PDAC) is increasingly treated with neoadjuvant chemotherapy and/or radiotherapyl, yet remains largely a treatment-refractory disease^{2,3}. Thus, there is an urgent need to decipher the impact of preoperative treatment on residual cancer cells and stroma to identify additional therapeutic vulnerabilities^{1,5}. Unlike many other cancers, PDAC molecular subtyping remains nascent and does not yet inform clinical management or therapeutic development^{6,7}. Bulk RNA profiling of PDAC tumors⁸⁻¹³ has identified two subtypes: (1) classical/epithelial, encompassing a spectrum of pancreatic lineage precursors, and (2) basal-like/squamous/quasi-mesenchymal, exhibiting loss of endodermal identity and genetic aberrations in chromatin modifiers⁶, poorer responses to chemotherapyl¹⁴, and worse survival. Additional efforts to refine this taxonomy did not further stratify patient survival^{6,10}, and other proposed subtypes (e.g., exocrine, aberrantly-differentiated endocrine exocrine (ADEX)), may reflect microenvironmental features⁸⁻¹¹. Moreover, most prior studies profiled tumors from untreated patients. Finally, while contributions of the tumor microenvironment (TME) may impact the effect of cytotoxic treatments¹⁵⁻¹⁷, motivating the use of adjunctive therapies such as losartan^{18-21.5}, the understanding of the spatial architecture and multicellular interactions in the TME remains limited.

Single-cell RNA-seq (scRNA-seq) can reveal the diversity of malignant and non-malignant cells in tumors²²⁻²⁶, and elucidate the impact of therapy on each compartment, but it has been challenging to apply in PDAC, given the high intrinsic nuclease content and dense desmoplastic stroma²⁷⁻³⁰ Single-nucleus RNA-seq (snRNA-seq) provides a compelling alternative that can be applied to frozen samples³¹⁻³⁴ and better recover malignant and stromal cells³⁵⁻³⁷, but has not yet been demonstrated in PDAC. Moreover, single cell profiles do not capture spatial context directly³⁸. Prior spatial proteotranscriptomic analyses of the PDAC TME were limited in molecular multiplexing^{39,40,41} or spatial resolution⁴²

In this Example, Applicant optimized snRNA-seq for banked frozen PDAC specimens stored up to five years, profiled 224,988 nuclei across 43 tumors (18 untreated, 25 treated), and recovered similar overall cellular compositions to those from multiplex protein profiling in situ. Applicant discovered treatment-associated changes in cellular composition and expression programs in the malignant, fibroblast, and immune compartments, including enrichment of a novel neural-like progenitor malignant program in residual tumor and patient-derived treated organoids. By integrating cell type signatures and expression programs with whole-transcriptome digital spatial profiles of matched specimens43, Applicant identified distinct intercellular interactions and multicellular communities. This Example at least provides a high-resolution map of the molecular composition of tumors remodeled under treatment selection pressures.

### Single-Nucleus RNA-Seq Accurately Captures the Malignant and Non-Malignant Compartments of Human PDAC Tumors

Applicant collected 224,988 high quality snRNA-seq profiles from flash frozen, histologically-confirmed, primary PDAC specimens from 43 patients (out of 48 in the study) who underwent surgical resection with (n=25) or without (n=18) neoadjuvant treatment (FIG. 1A; Table 2, Methods). Most treated patients had received multiple cycles of cytotoxic chemotherapy (FOLFIRINOX) followed by multi-fraction radiotherapy with concurrent 5-FU or capecitabine (CRT; n=14; Table 2). Five additional patients were enrolled on a therapeutic interventional clinical trial (NCT01821729) investigating neoadjuvant CRT with the addition of losartan (CRTL; n=5). Another six received other forms of neoadjuvant treatment, including two on a phase 2 randomized clinical trial (NCT03563248) who received FOLFIRINOX, stereotactic body radiotherapy, and nivolumab with (CRTLN; n=1) or without losartan (CRTN; n=1) (Table 2).

TABLE 2
Patient cohort and clinicopathologic data.

| | | | | | | |
|---|---|---|---|---|---|---|
| | Age | | | | | |
| ID | (decade) | Sex | Stage/Grade | Margin | Histology | Neoadjuvant |

Untreated (U)

| | | | | | | |
|---|---|---|---|---|---|---|
| PDAC_U_1 | 20s | F | T3N1M0/g2-3 | R1 | | None |
| PDAC_U_2 | 60s | F | T3N2M0/g2 | R1 | AS | None |
| PDAC_U_3 | 60s | F | T3N0M0/gX | R0 | FG | None |
| PDAC_U_4 | 60s | M | T3N1M0/g3 | R0 | | None |
| PDAC_U_5 | 60s | M | T3N1M0/g3 | R1 | | None |
| PDAC_U_6 | 60s | M | T2N2M0/g2 | R0 | | None |
| PDAC_U_7 | 70s | M | T3N2M0/g2 | R0 | AS | None |
| PDAC_U_8 | 70s | F | T2N1M0/g2 | R0 | | None |
| PDAC_U_9 | 70s | M | T2N1M0/g2 | R0 | | None |
| PDAC_U_10 | 70s | M | T3N0M0/g2-3 | R1 | | None |
| PDAC_U_11 | 70s | F | T2N1M0/g2 | R1 | | None |
| PDAC_U_12 | 70s | F | T2N1M0/g2 | R1 | | None |
| PDAC_U_13 | 70s | M | T2N0M0/g2-3 | R1 | | None |
| PDAC_U_14 | 70s | M | T3N0M0/g2 | R1 | | None |
| PDAC_U_15 | 80s | F | T2N0M0/g2 | R0 | | None |
| PDAC_U_16 | 80s | M | T2N1M0/g2-3 | R1 | | None |
| PDAC_U_17 | 80s | F | T2N1M0/g2 | R1 | FG | None |
| PDAC_U_18 | 80s | M | T3N2M0/g3 | R1 | AS | None |
| PDAC_U_19^{†} | 60s | F | T2N0M0/g2-3 | R1 | | None |
| PDAC_U_20^{†} | 70s | F | T2N2M0/g2 | R0 | | None |
| PDAC_U_21^{†} | 70s | F | T2N0M0/g2-3 | R0 | | None |

Treated (T)

| | | | | | | |
|---|---|---|---|---|---|---|
| PDAC_T_1 | 50s | M | ypT2N0M0/gX | R0 | | CRT |
| PDAC_T_2 | 50s | F | ypT3N0M0/g2 | R0 | | CRT |
| PDAC_T_3 | 60s | F | ypT1cN1M0/g2 | R1 | | CRT |
| PDAC_T_4 | 60s | F | ypT3N0M0/g2 | R0 | | CRT |
| PDAC_T_5 | 60s | F | ypT2N0M0/gX | R1 | | CRT |
| PDAC_T_6 | 60s | M | ypT2N2M0/g3 | R0 | AS | CRT |
| PDAC_T_7 | 60s | M | ypT1cN0M0/g2 | R0 | | CRT |
| PDAC_T_8 | 60s | M | ypT2N0M0/gX | R0 | | CRT |
| PDAC_T_9 | 70s | F | ypT3N0M0/gX | R0 | | CRT |
| PDAC_T_10 | 70s | F | ypT1aN0M0/gX | R0 | | CRT |
| PDAC_T_11 | 70s | M | ypT2N1M0/g3 | R0 | FG | CRT |
| PDAC_T_12 | 70s | M | ypT2N0M0/g2 | R1 | | CRT |
| PDAC_T_13 | 70s | M | ypT3N0M0/g2 | R0 | | CRT |
| PDAC_T_14 | 70s | M | ypT3N0M0/g2 | R0 | | CRT |
| PDAC_T_15 | 50s | M | ypT1aN0M0/g2 | R0 | | CRTL** |
| PDAC_T_16 | 60s | M | ypT3N0M0/gX | R1 | | CRTL** |
| PDAC_T_17 | 70s | F | ypT3N0M0/g2 | R0 | | CRTL** |
| PDAC_T_18 | 70s | M | ypT3N0M0/g2 | R0 | | CRTL** |
| PDAC_T_19 | 80s | F | ypT3N1M0/gX | R0 | | CRTL** |
| PDAC_T_20 | 60s | F | ypT1aN1M0/gX | R0 | | CRTN* |
| PDAC_T_21 | 60s | F | ypT2N0M0/g2 | R0 | | CRTLN* |
| PDAC_T_22 | 30s | F | ypT2N0M0/g2 | R0 | BRCA2 germ | Other |
| PDAC_T_23 | 50s | M | ypT1cN0M0/g3 | R1 | BRCA2 germ | Other |
| PDAC_T_24 | 60s | F | ypT3N1M0/gX | R0 | | Other |
| PDAC_T_25 | 70s | F | ypT2N1M0/g3 | R0 | | Other |
| PDAC_T_26^{†} | 70s | F | ypT2N0M0/g2 | R1 | | CRT |
| PDAC_T_27^{†} | 70s | M | ypT2N2M0/gX | R0 | | Other |

| | | | | | |
|---|---|---|---|---|---|
| | Status | | | | |
| | Last | PFS | OS | Storage | 10x |
| ID | FUP | (d) | (d) | (d) | Chemistry |

Untreated (U)

| | | | | | |
|---|---|---|---|---|---|
| PDAC_U_1 | DWD | 70 | 919 | 638 | v3 |
| PDAC_U_2 | DWD | 78 | 297 | 17 | v3 |
| PDAC_U_3 | MET | 578 | 1082 | 341 | v3 |
| PDAC_U_4 | DWD | 267 | 538 | 634 | v2 |
| PDAC_U_5 | NED | 1338 | 1338 | 661 | v3 |
| PDAC_U_6 | MET | 466 | 529 | 220 | v3 |
| PDAC_U_7 | DWD | 208 | 563 | 384 | v3 |
| PDAC_U_8 | NED | 385 | 385 | 335 | v3 |
| PDAC_U_9 | MET | 35 | 607 | 62 | v3 |
| PDAC_U_10 | DWOD | 494 | 494 | 472 | v3 |
| PDAC_U_11 | NED | 1057 | 1057 | 393 | v3 |
| PDAC_U_12 | DWOD | 172 | 172 | 64 | v3 |
| PDAC_U_13 | DWD | 329 | 850 | 135 | v2 |
| PDAC_U_14 | DWD | 255 | 404 | 106 | v3 |
| PDAC_U_15 | NED | 554 | 554 | 38 | v2 |
| PDAC_U_16 | DWOD | 223 | 223 | 169 | v3 |
| PDAC_U_17 | DWD | 59 | 405 | 335 | v3 |
| PDAC_U_18 | DWD | — | 171 | 148 | v3 |
| PDAC_U_19^{†} | NED | 18 | 18 | 475 | N/A |
| PDAC_U_20^{†} | NED | 321 | 321 | 280 | N/A |
| PDAC_U_21^{†} | NED | 112 | 112 | 287 | N/A |

Treated (T)

| | | | | | |
|---|---|---|---|---|---|
| PDAC_T_1 | DWD | 188 | 612 | 586 | v3 |
| PDAC_T_2 | NED | 1811 | 1811 | 1121 | v3 |
| PDAC_T_3 | DWD | — | 91 | 662 | v3 |
| PDAC_T_4 | NED | 1797 | 1797 | 1142 | v3 |
| PDAC_T_5 | DWD | 187 | 890 | 104 | v2 |
| PDAC_T_6 | MET | 34 | 34 | 112 | v2 |
| PDAC_T_7 | DWD | 117 | 345 | 56 | v2 |
| PDAC_T_8 | NED | 1813 | 1813 | 1119 | v3 |
| PDAC_T_9 | MET | 1350 | 1865 | 1701 | v3 |
| PDAC_T_10 | NED | 624 | 624 | 523 | v3 |
| PDAC_T_11 | NED | 39 | 39 | 6 | v3 |
| PDAC_T_12 | NED | 391 | 391 | 516 | v3 |
| PDAC_T_13 | MET | 310 | 849 | 90 | v3 |
| PDAC_T_14 | NED | 201 | 201 | 52 | v3 |
| PDAC_T_15 | NED | 1111 | 1111 | 998 | v3 |
| PDAC_T_16 | NED | 1125 | 1125 | 497 | v3 |
| PDAC_T_17 | NED | 1536 | 1536 | 1023 | v3 |
| PDAC_T_18 | DWD | 258 | 362 | 905 | v3 |
| PDAC_T_19 | MET | 900 | 1171 | 1164 | v3 |
| PDAC_T_20 | NED | 664 | 664 | 58 | v3 |
| PDAC_T_21 | MET | 247 | 372 | 512 | v3 |
| PDAC_T_22 | MET | 560 | 769 | 69 | v3 |
| PDAC_T_23 | DWD | 285 | 355 | 170 | v3 |
| PDAC_T_24 | DWD | 62 | 76 | 364 | v3 |
| PDAC_T_25 | DWD | 110 | 185 | 365 | v3 |
| PDAC_T_26^{†} | NED | 111 | 111 | 14 | N/A |
| PDAC_T_27^{†} | NED | 182 | 182 | 8 | N/A |

Abbreviations: AS, adenosquamous; FG, foamy gland variant; BRCA2 germ, BRCA2 germline mutation; MET, distant metastases; LR, local recurrence; DWD, dead with disease; DWOD, dead without evidence of disease; NED, no evidence of disease; CRT, FOLFIRINOX + radiotherapy with concurrent capecitabine or 5-FU; CRTL, FOLFIRINOX + losartan + radiotherapy with concurrent capecitabine or 5-FU; CRTN, FOLFIRINOX + stereotactic body radiotherapy + nivolumab; CRTLN, FOLFIRINOX + stereotactic body radiotherapy + losartan + nivolumab; Other, treatment regimen consisting of chemotherapy and/or radiotherapy combination not otherwise specified;
^{†}No snRNA-seq (DSP only);
*NCT03563248;
**NCT01821729

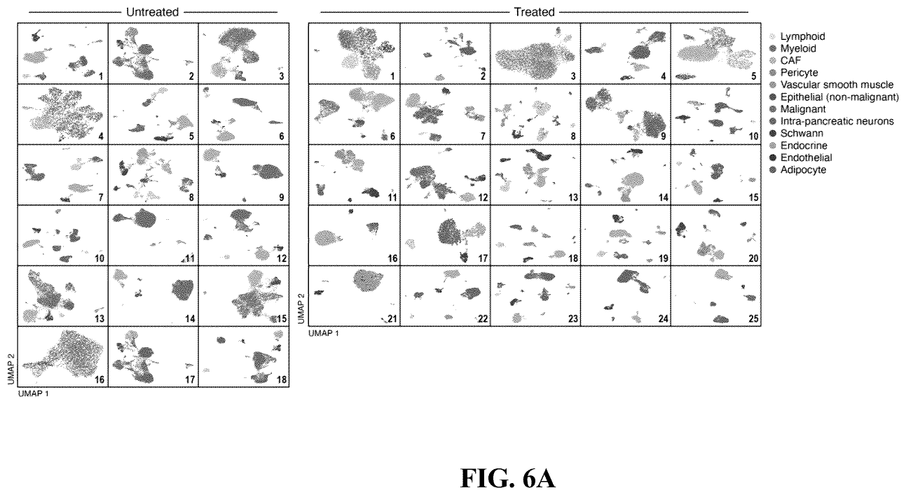

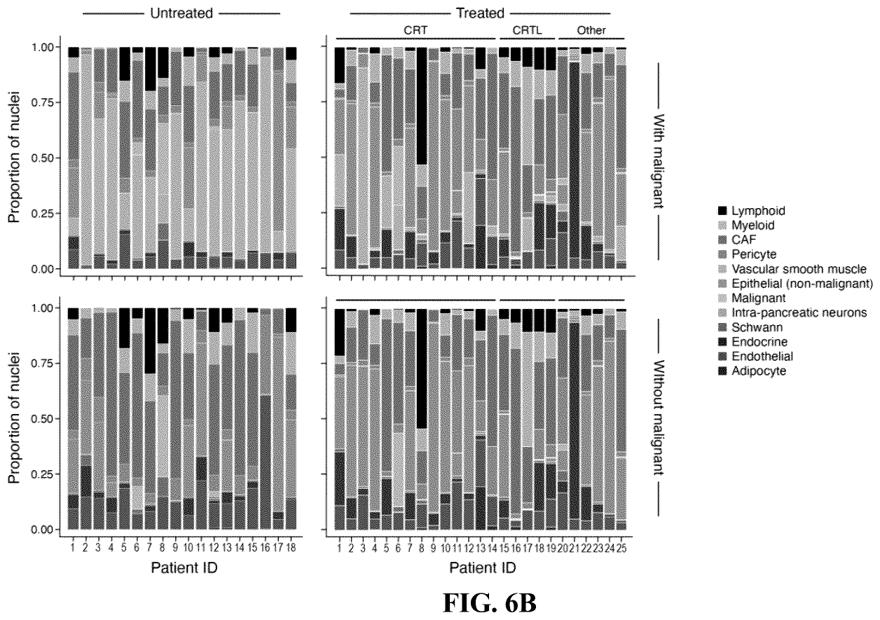

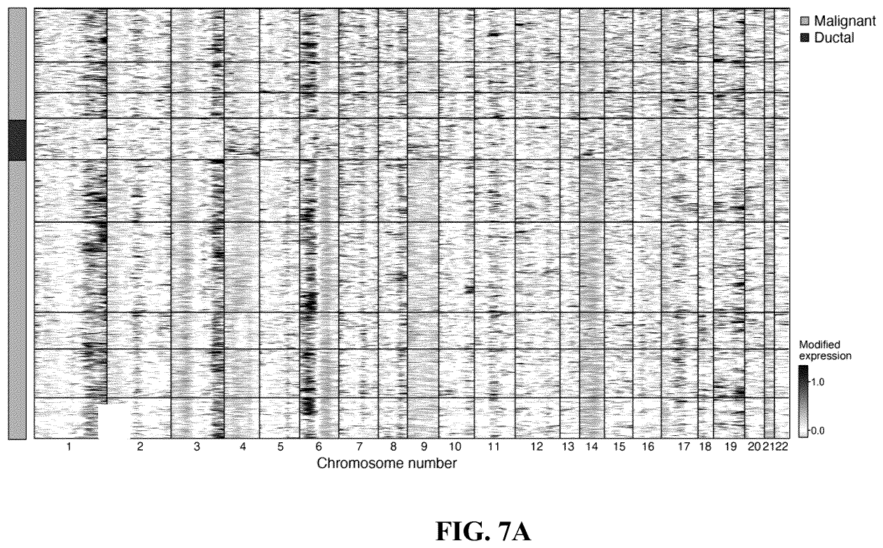

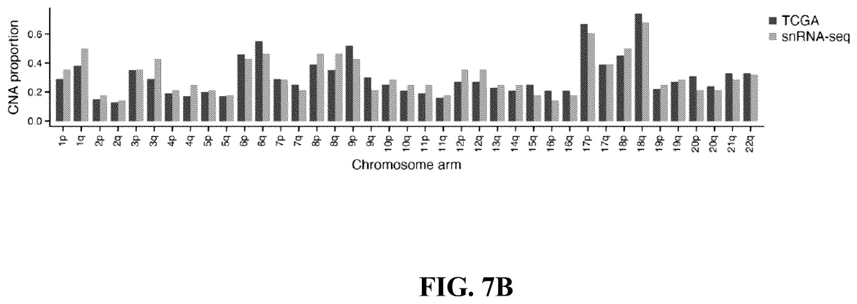

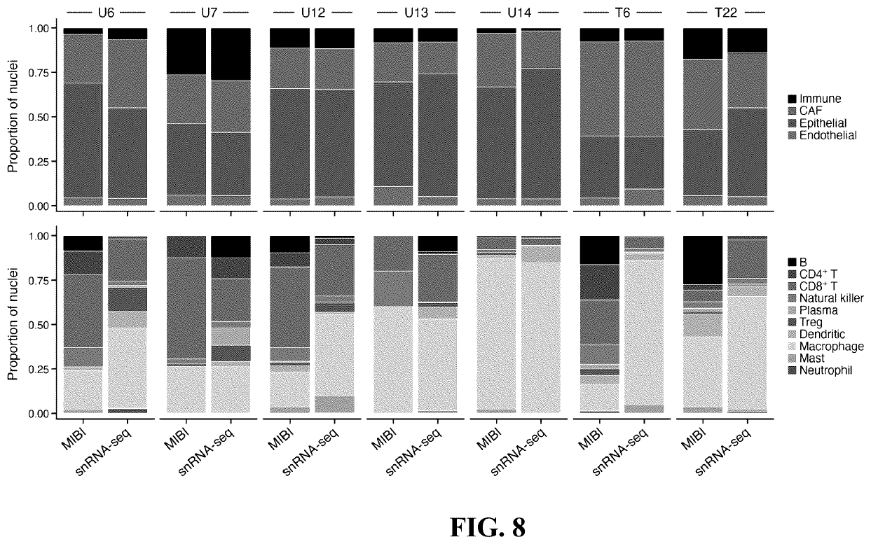

Unsupervised clustering of single nucleus profiles identified 33 cell subsets, which Applicant annotated post hoc by known gene signatures (FIG. 1B-1D; FIG. 6; Methods)^{34,44-47} Applicant confirmed malignant cells by inferred Copy Number Alterations (CNAs), which were comparable to those derived from The Cancer Genome Atlas (TCGA) PDAC cohort (FIG. 7A)^{11,22}, and generally clustered by patient (FIG. 6B-6C; adjusted mutual information (AMI)=0.87 in malignant cells vs. 0.18 in non-malignant cells). Other cell types included non-malignant epithelial cells, immune, endocrine, and diverse stromal cells (cancer-associated fibroblasts/CAFs, endothelial cells, vascular smooth muscle cells, pericytes, intra-pancreatic neurons, Schwann cells, and adipocytes) (FIGS. 6B, and 6D). Cell types like CAFs, previously under-represented in scRNA-seq⁴⁸-51, were well-represented in the samples (FIGS. 1B and 1D; FIG. 6). snRNA-seq captured representative distributions of epithelial, fibroblast, endothelial, and immune cell type proportions compared to estimates from multiplexed ion beam imaging (MIBI) across (FIG. 1E) and within (FIG. 8) individual tumors, but with some differential capture within certain immune cell subsets (FIG. 1E; FIG. 8; Methods)³⁷.

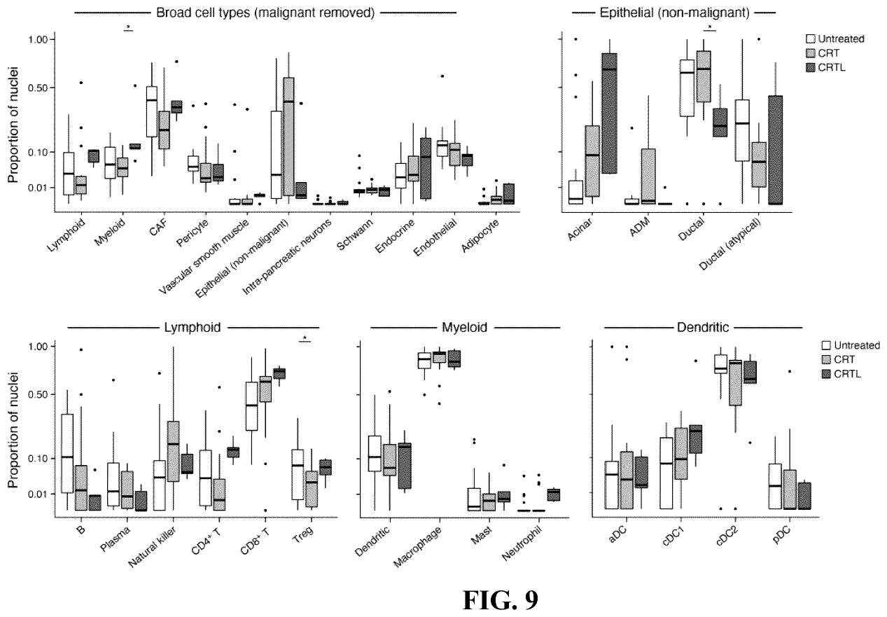

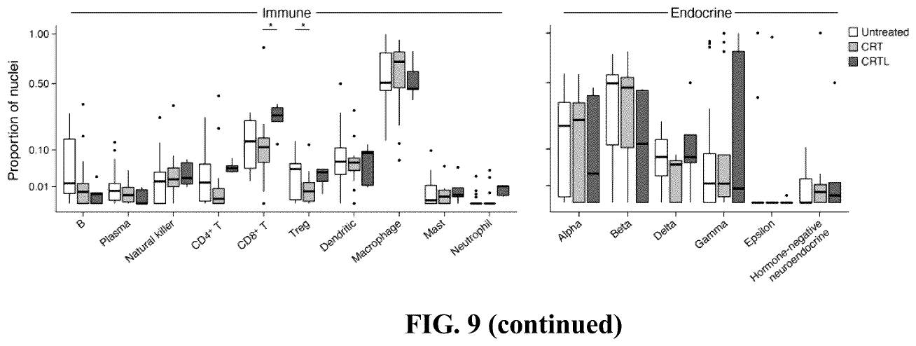

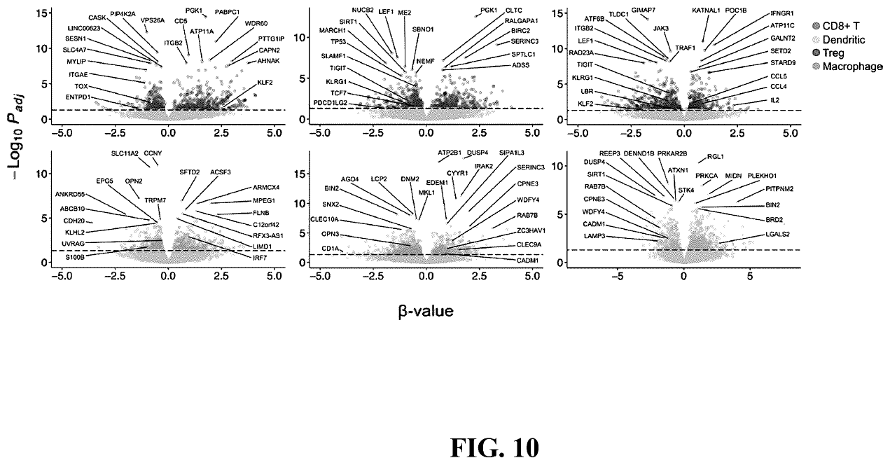

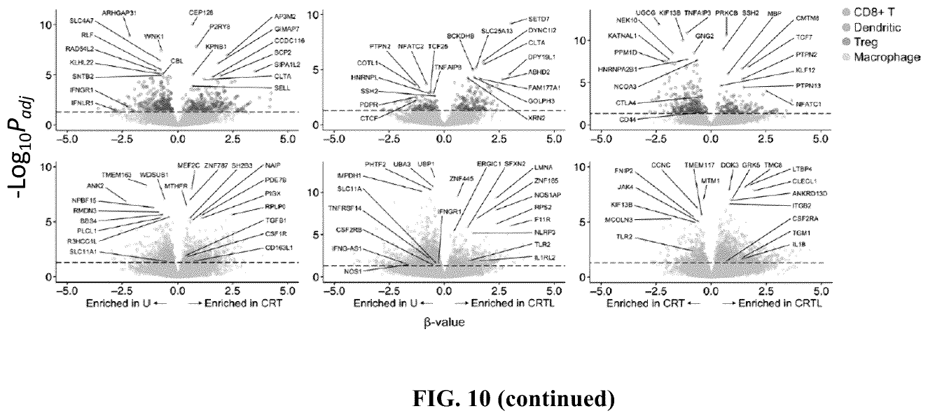

Among clinical subgroups with at least five patients (untreated, CRT, CRTL), malignant cell proportions were significantly lower in tumors treated with neoadjuvant therapy (CRT vs. untreated, padi=1.16×10⁻³; CRTL vs. untreated, padj=3.81×10⁻³ by Mann-Whitney U test and pᵢ>0.99 by Dirichlet-multinomial regression; FIG. 1F), consistent with histology. Several non-malignant cell subsets differed quantitatively and qualitatively across treatment groups (FIG. 1F; FIG. 9-11; Methods). For example, within the immune compartment, there was a higher fraction of CD8⁺ T cells in neoadjuvant CRTL vs. CRT (padj=3.51×10⁻²; Mann-Whitney U test) (FIG. 9), and a lower proportion of regulatory T cells (T_{regs}) in CRT vs. untreated (p_{adj}=2.27×10⁻²; Mann-Whitney). Moreover, CD8⁺ T cells in CRTL tumors expressed higher levels of effector function genes (e.g., IL2, CCL4, CCL5)^{52,53} and lower levels of quiescence and dysfunction genes (e.g., TIGIT, TCF7, KLF2, LEF1)⁵⁴⁻⁶⁰ vs. untreated and CRT tumors (FIG. 10; Methods). These results are consistent with the previously identified losartan-mediated increase in intra-tumoral cytotoxic T cell activity^{20,61}

### Acinar-to-Ductal Metaplasia (ADM) and Atypical Ductal Cells are Putative Intermediate States in PDAC Development

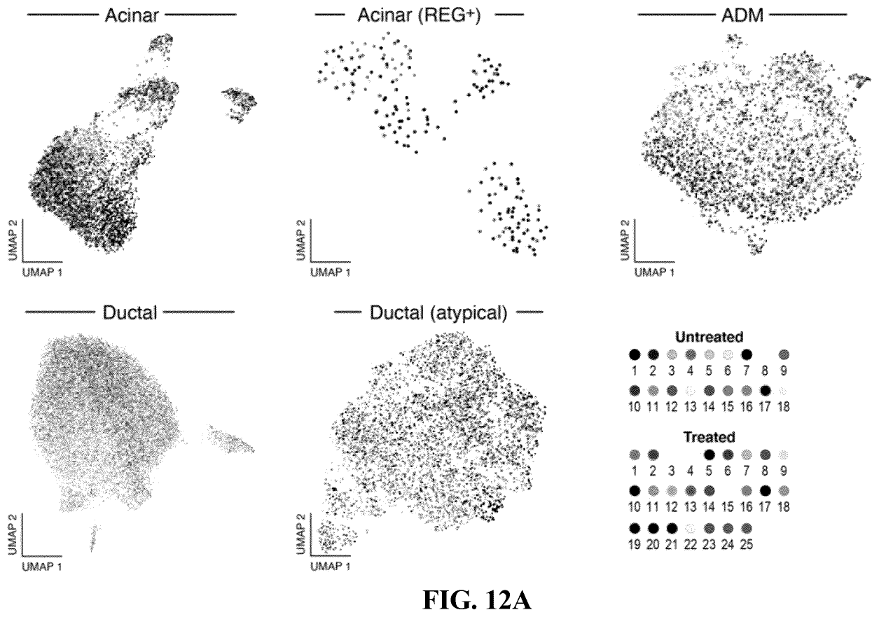

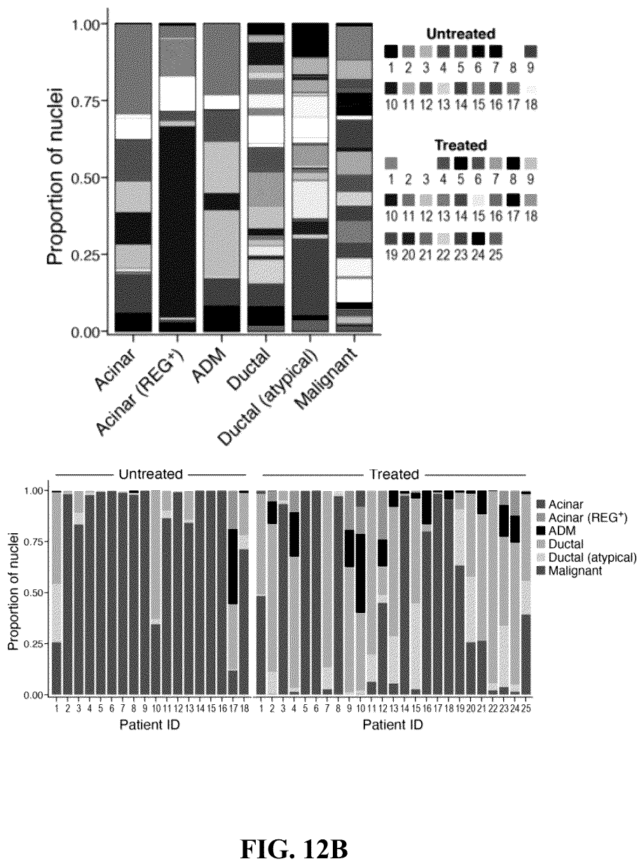

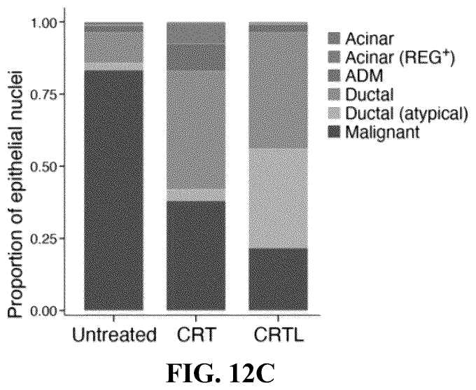

Within the epithelial compartment, there were low CNA nuclei co-expressing markers of ductal and acinar lineages (FIGS. 1B and 1D; FIG. 12) that may reflect acinar-to-ductal metaplasia (ADM), which has been shown to play an initiating role in mouse pancreatic tumorigenesis^{62,63}. These profiles had a typical number of transcripts and are unlikely to be doublets. Chronic inflammation and somatic KRAS mutations have been linked to persistence of the ADM state and progression to pre-invasive pancreatic intraepithelial neoplasia (PanIN)^{63,64} Consistently, the ADM cells had higher expression of the HALLMARK_KRAS_SIGNALING_UP signature65 compared to acinar cells (FIG. 1G).

Moreover, a distinct subset of ductal cells expressed high levels of both ductal (e.g., (FTR) and malignant (e.g., KRT5, KRT17, KRT19) markers without elevated CNAs, which Applicant termed atypical ductal cells (FIG. 1B). Atypical ductal cells featured genes (e.g., KRT17)⁶⁶ that are expressed as early as the PanIN2/3 stage and had higher levels of the HALLMARK_KRAS_SIGNALING_UP signature than ADM cells, suggesting a progression from ADM to precursor lesions such as PanINs (FIGS. 1B, 1D, and 1G; FIG. 12). Indeed, a partition-based graph abstraction (PAGA)⁶⁷ inferred a dominant pseudotemporal trajectory from acinar to ADM to ductal to atypical ductal to malignant cells (FIG. 1H), paralleling a monotonic increase in the HALLMARK_KRAS_SIGNALING_UP signature, supporting ADM and atypical ductal cells as relevant intermediate states in PDAC tumorigenesis (FIGS. 1G and 1H).

### Malignant and Fibroblast Programs Shared Across Tumors

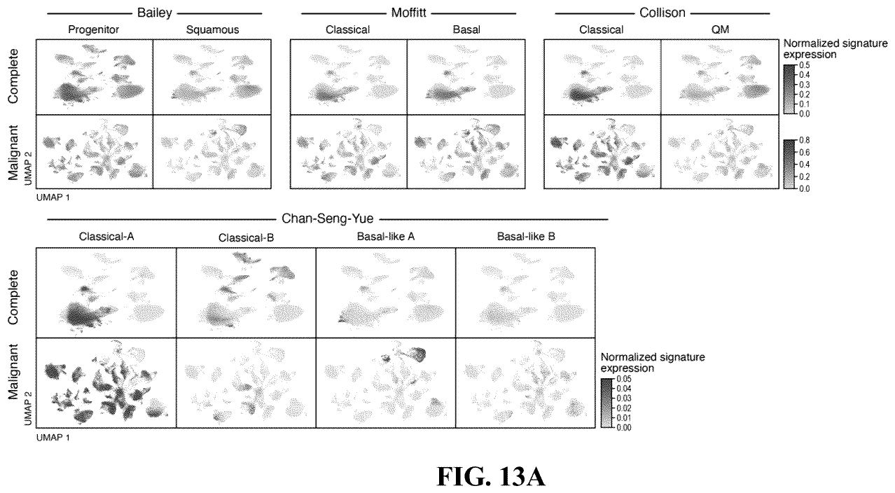

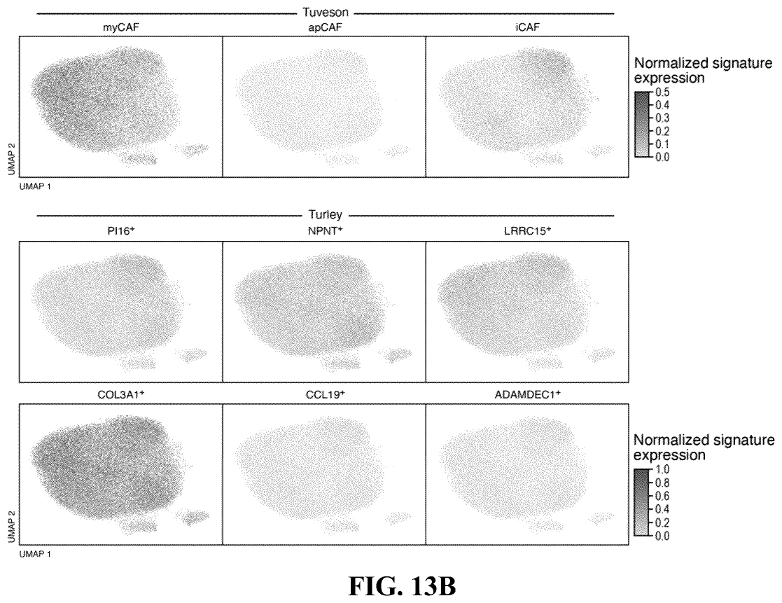

Prior expression signatures of epithelial- or CAF-enriched PDAC tumors only partially aligned with the partitioning of Applicant's single nucleus profiles (FIG. 13A-13B). Although most tumors had malignant cells of the basal-like/squamous/quasi-mesenchymal and classical subtypes (FIG. 13A; Methods)^{68,69}, these states overlapped in some malignant cells^{69,70}, or were absent in others. Moreover, myofibroblastic (myCAF) and inflammatory (iCAF) CAF signatures were expressed in somewhat distinct subsets of CAFs, but the antigen-presenting (apCAF) signature was not clearly identified (FIG. 13B; Methods)^{49,71}, and cross-tissue signatures⁷² only partially segregated the CAF profiles (FIG. 13B).

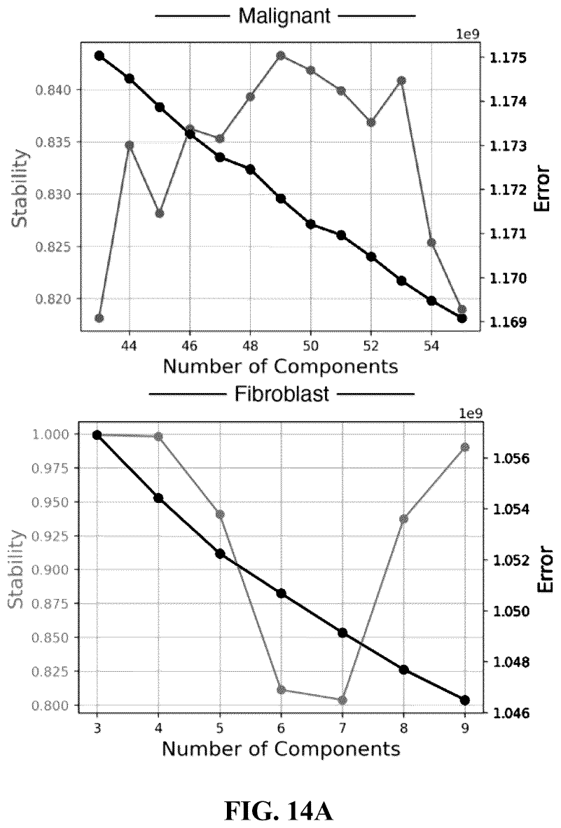

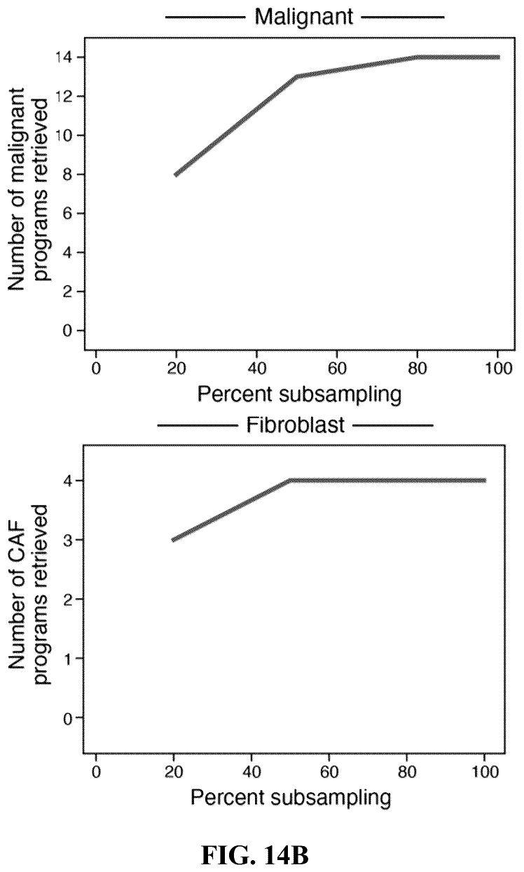

Applicant therefore learned recurrent expression programs de novo across malignant cells and CAFs of different tumors, using consensus non-negative matrix factorization (cNMF)⁷³. Applicant selected the number of cNMF programs by stability and error (FIG. 14A), focused on those shared across cells from multiple patients (FIG. 2A; Tables 4-6; Methods), and annotated each by its top-200 weighted genes (Methods).

Applicant identified 14 malignant cell programs that reflected either lineage (classical-like, squamoid, basaloid, mesenchymal, acinar-like, neuroendocrine-like, neural-like progenitor) or cell state (cycling(S), cycling (G2/M), MYC, interferon, TNF-NFκB, ribosomal, adhesive) (FIG. 2A, Tables 4-5), and four CAFs programs: myofibroblastic progenitor, neurotropic, immunomodulatory, and adhesive (FIG. 2A). Subsampling of tumors showed recovery all 14 malignant programs when using 80% of samples and all four fibroblast programs at 50% subsampling (FIG. 14B).

TABLE 4
Top 200 weighted genes for malignant and fibroblast cNMF programs.

| | Malignant state programs | | | | | | |
|---|---|---|---|---|---|---|---|
| Gene | Cycling | Cycling | MYC | | | Interferon | TNF-NFkB |
| rank | (S) | (G2/M) | signaling | Adhesive | Ribosomal | signaling | signaling |
| 1 | HELLS | ASPM | PVT1 | LAMB3 | RPS17 | POU6F2 | LAMC2 |
| 2 | BRIP1 | CENPE | WDR43 | CD55 | RPS15A | HECW2 | CDH2 |
| 3 | DTL | TOP2A | CMSS1 | EMP1 | RPS23 | BCAT1 | ZNF365 |
| 4 | ATAD2 | KIF14 | PUM3 | SGMS2 | RPS13 | IGSF1 | PODXL |
| 5 | FANCI | PIF1 | LRPPRC | GPRC5D | RPL32 | SOX5 | COL22A1 |
| 6 | FAM111B | BUB1 | PUS7 | PTCHD4 | ZNF90 | MUC16 | ETS1 |
| 7 | FANCA | GTSE1 | DDX10 | ERCC1 | RPS12 | AC026167.1 | TGFBI |
| 8 | CENPK | TPX2 | IARS | LMNA | RPL34 | LYPD2 | RTKN2 |
| 9 | MYBL2 | KIF18B | DDX21 | SEMA4A | RPL35A | CDH4 | CXCL8 |
| 10 | MELK | ANLN | URB1 | RGCC | RPL19 | HDAC9 | CDK14 |
| 11 | POLQ | CENPF | NDUFAF2 | NEDD9 | MAMDC2 | GNGT1 | ITGAV |
| 12 | DIAPH3 | DLGAP5 | SCFD2 | ADGRE5 | RPS14 | ALOX12-AS1 | SEMA7A |
| 13 | CLSPN | CIT | BZW2 | EZR | OOEP | IFI44L | PALLD |
| 14 | WDR76 | KIF4A | NOP58 | GC | RPS18 | NCALD | TSPAN2 |
| 15 | BRCA2 | KIF23 | UCK2 | LINC01411 | RPS15 | MYO16-AS1 | IL12RB1 |
| 16 | CENPP | NUSAP1 | PRMT3 | FA2H | RPS4X | MUC4 | NEURL1B |
| 17 | ATAD5 | CDC25C | ACACA | LMO7 | RPL27A | VWA5B1 | PLAU |
| 18 | NCAPG2 | CDCA2 | POLR1A | RHPN2 | RPL27 | NXPH1 | SAMD4A |
| 19 | ZGRF1 | KIF18A | FAM208B | SYNE1 | RPL11 | ZNF83 | TPM1 |
| 20 | BRCA1 | KIF11 | PRKDC | SLC7A5 | RPS5 | TRIM22 | RASGRP1 |
| 21 | WDHD1 | CCNF | METTL8 | SERPINB1 | RPS25 | AC011306.1 | DCBLD2 |
| 22 | UHRF1 | KIF20B | SNHG15 | IGFBP1 | RPS27A | ZBP1 | TNFRSF11B |
| 23 | EXO1 | HJURP | HEATR1 | BTBD19 | RPL7 | CCDC146 | PDGFB |
| 24 | MCM10 | ARHGAP11A | FARSB | CD9 | RPLP1 | AC024084.1 | F3 |
| 25 | CDC45 | NCAPG | PPAT | KCNK1 | RPL23A | TDRD1 | PHLDB2 |
| 26 | CENPU | HMMR | ABCE1 | ARL10 | RPS8 | KYNU | SEMA3C |
| 27 | XRCC2 | AURKA | CEP83 | PLAT | RPL36 | EPSTI1 | CASC15 |
| 28 | ZNF367 | CCNB1 | PNPT1 | MYO1E | RPL29 | NR2F2-AS1 | LAMB3 |
| 29 | GINS1 | CDK1 | PAICS | SLC20A1 | RPL38 | GLI2 | CHST11 |
| 30 | RFC3 | IQGAP3 | ATR | ELP5 | RPL14 | SAMD9L | TNFAIP3 |
| 31 | RBL1 | NDC80 | EIF3B | HRH1 | RPS16 | GPCPD1 | ADAMTS9 |
| 32 | CENPI | DEPDC1 | TSEN2 | FOSL1 | RPL21 | PCDH7 | TNF |
| 33 | EZH2 | CEP55 | R3HDM1 | SOX5 | RPL30 | IKZF2 | FGD6 |
| 34 | MMS22L | TROAP | WDR3 | HK2 | EFCAB3 | SLC15A2 | ITGB1 |
| 35 | BLM | MKI67 | GPHN | ITPKC | RPL7A | KCNQ3 | PGBD5 |
| 36 | KNTC1 | NUF2 | SND1 | ESYT2 | FAU | MBOAT2 | FRMD6 |
| 37 | RAD54L | BUB1B | OLA1 | HBEGF | COX7C | FBXO34 | EGOT |
| 38 | POLA1 | KIF2C | MDN1 | ARHGAP30 | NACA2 | IFI44 | ABTB2 |
| 39 | DNA2 | CKAP2 | MRPL3 | TPM4 | RPL15 | PLCB1 | ABLIM3 |
| 40 | RAD51AP1 | PRR11 | RCC1 | MPRIP | UBA52 | XAF1 | MICAL2 |
| 41 | E2F1 | FAM83D | PDCD11 | GPRC5A | RPL13A | ADAMTS16 | MICAL3 |
| 42 | FANCB | DEPDC1B | TTC27 | SFRP4 | RPL24 | NRP2 | FSTL3 |
| 43 | E2F7 | RACGAP1 | ANAPC1 | LGALS3 | RPLP2 | IGF2BP3 | SLC30A4 |
| 44 | CHAF1A | TTK | NAA25 | ATF3 | RPS2 | PCLO | GLS |
| 45 | TYMS | KIF15 | IPO7 | BAIAP2 | RPL28 | GABRB3 | EDIL3 |
| 46 | LIG1 | CDCA3 | DDX31 | S100A10 | RPS3 | CACNA1C | ANKLE2 |
| 47 | ASF1B | CENPI | MATR3 | GPR132 | RPS19 | C1GALT1 | CDH6 |
| 48 | POLE2 | CKAP2L | C15orf41 | CAMK1G | RPS6 | LYN | CREB5 |
| 49 | RECQL4 | TACC3 | NAA15 | ANXA1 | RPL35 | EML4 | CDH1 |
| 50 | MCM4 | UBE2C | FIRRE | ATP2B4 | RPL31 | MACC1 | IRAK2 |
| 51 | DSCC1 | DIAPH3 | HSPD1 | CDCP1 | RPS7 | LRMP | CSF2 |
| 52 | BARD1 | CDCA8 | ZCCHC7 | CEACAM1 | RPL18A | SLCO3A1 | EGF |
| 53 | ORC6 | NEIL3 | XPO4 | ANXA11 | RPS3A | PLCH1 | TGFBR1 |
| 54 | MND1 | GPSM2 | NOC3L | LDLR | RPL23 | TCF20 | TNFAIP8 |
| 55 | CEP128 | TICRR | POLR1B | RFX2 | APOO | SAMD9 | GRB10 |
| 56 | POLE | SMC4 | MTHFD1L | GPR82 | FTL | NMUR2 | RALA |
| 57 | CCNE2 | APOLD1 | TAF1D | ABHD17C | TXNRD1 | ARHGAP12 | ITGB8 |
| 58 | VRK1 | MELK | WDR12 | YWHAZ | RPL8 | MAP3K13 | NAV2 |
| 59 | MCM8 | LMNB1 | CAMKMT | MCL1 | COMMD6 | RARRES3 | KLHL5 |
| 60 | ESCO2 | BORA | CNNM1 | CORO1C | RPL41 | LINC01376 | ARNTL2 |
| 61 | CDC6 | ECT2 | CDK6 | AC132807.1 | BDNF-AS | SCEL | SOX4 |
| 62 | DNMT1 | NCAPH | WDR4 | RAB11FIP1 | RPS27 | KAZN | PTAFR |
| 63 | CHEK1 | CDKN3 | CARNMT1 | ZBTB20-AS1 | RPL18 | STAT1 | MGAT4A |
| 64 | TCF19 | POLQ | GART | ITGA6 | RPS20 | ANKRD13A | ARHGAP31 |
| 65 | MCM6 | ESPL1 | AGPAT5 | VPS37B | RPS28 | SLC15A1 | FRMD5 |
| 66 | POLA2 | INCENP | EIF3E | SYNJ2 | NHSL2 | DOCK8 | KIF3C |
| 67 | IQGAP3 | AURKB | ESF1 | STK 40 | RPL3 | AGR3 | MACC1 |
| 68 | ZWINT | KIFC1 | G3BP1 | PLK3 | RPL6 | CASC9 | ITGA2 |
| 69 | E2F2 | PARPBP | NOL10 | PELI1 | RPS11 | ABCA12 | C15orf48 |
| 70 | PRIM2 | PLK1 | ABCC1 | BAIAP2L1 | KIZ-AS1 | RORA | CEP170 |
| 71 | C21orf58 | STIL | DKC1 | MDM2 | UQCRB | POU6F2-AS2 | SLCO2A1 |
| 72 | PKMYT1 | PBK | WWC1 | PER2 | RPS24 | PARP14 | PTPRE |
| 73 | FANCD2 | CCDC18 | FRAS1 | TIPARP | EEF1A1 | APOL6 | BCAR3 |
| 74 | RRM1 | CKAP5 | NARS2 | PLAUR | SNRPD2 | NCF4 | KLF7 |
| 75 | RAD54B | SCLT1 | EIF3H | MICAL2 | TOMM7 | B3GALT5 | LIMS1 |
| 76 | NCAPD3 | CCDC150 | CTPS1 | NRP2 | RPL37A | CFH | TRIO |
| 77 | ANLN | KPNA2 | EIF2B3 | MPRIP-AS1 | RPLP0 | CDH26 | LINC01239 |
| 78 | TK1 | PTTG1 | TCERG1 | SHANK2-AS1 | RPS9 | IRAK3 | VGLL3 |
| 79 | LMNB1 | CDC20 | NAT10 | EPHA2 | RPL13 | PLCL2 | C3orf52 |
| 80 | TP73 | BIRC5 | TEAD4 | C6orf132 | ADIRF | B4GALT5 | MMP9 |
| 81 | RFC4 | OTUD7A | NOP56 | MYADM | SLC47A1 | PLEKHG7 | MMP10 |
| 82 | KIF24 | KNSTRN | PPARGC1B | NAMPT | UQCRHL | SNX13 | SLC22A3 |
| 83 | TICRR | NEK2 | UBE2G2 | SLAMF7 | XPO5 | SP140L | HDAC9 |
| 84 | NCAPG | DBF4B | NFXL1 | RHOD | RPL39 | OAS1 | KCNA7 |
| 85 | KIF15 | PSRC1 | UTP20 | PTPRN2 | RPS29 | PARP9 | TMEM132A |
| 86 | CIT | SHCBP1 | WDR36 | B4GALNT3 | OST4 | ISG15 | TIAM2 |
| 87 | DSN1 | LINC01572 | KITLG | EGR3 | RPSA | GNA14 | MAP4K4 |
| 88 | NEIL3 | NCAPD2 | DCAF13 | ACTN4 | BTF3 | MRVI1-AS1 | HIVEP1 |
| 89 | STIL | CENPA | LYRM4 | MUC13 | COX4I1 | SP140 | MIR193BHG |
| 90 | MTBP | MIS18BP1 | SNHG8 | UBE2H | RPL37 | SREBF2 | SVIL |
| 91 | WDR62 | C21orf58 | ADK | CDH1 | ALDOA | CLIC5 | SUSD6 |
| 92 | CNTLN | NMU | ASAP1 | KLK7 | S100A2 | TOX3 | NAV1 |
| 93 | C2orf48 | HMGB2 | TARBP1 | RASEF | FTH1 | TMEM106B | DUSP10 |
| 94 | MYBL1 | KIF20A | IMMP2L | SNX9 | NPC2 | ARL17B | IL18 |
| 95 | SLF1 | DBF4 | DNAJC2 | CTNNB1 | RPL4 | NPSR1 | WNT7A |
| 96 | NASP | GAS2L3 | UBE3C | SEPT9 | PTGES | PACSIN2 | FNDC3B |
| 97 | SPC25 | CCNA2 | PMM2 | PCSK5 | RPL10A | TRIM6 | CLIP4 |
| 98 | WDR90 | SKA3 | XPOT | MIDN | COX6B1 | EIF2AK2 | MFGE8 |
| 99 | MCM5 | CCNB2 | TIMM23B | SEMA3B | GAPDH | PTPRR | EFNB2 |
| 100 | CEP152 | CEP128 | DAP3 | FAM102A | TPT1 | NLRC5 | ELK3 |
| 101 | KIF18B | RGS3 | TRAP1 | CDHR2 | RPL10 | RAB8B | MYO1E |
| 102 | KIF11 | GEN1 | GCFC2 | MXD1 | HINT1 | SLC2A3 | SERPINE1 |
| 103 | CDCA5 | FOXM1 | NDC1 | ABHD2 | IGFBP6 | SLC44A5 | PMEPA1 |
| 104 | SMC4 | SPC25 | HS6ST2 | CPM | SLC25A6 | CREG2 | PDLIM7 |
| 105 | MCM3 | WDR62 | NOLC1 | LGALS2 | MZT2B | DTX2 | CDA |
| 106 | GINS4 | NCAPG2 | SF3B3 | GLUD1 | RPL12 | SKAP1 | THBS1 |
| 107 | TONSL | ARHGEF39 | SRM | AC023590.1 | RPL36AL | PIAS1 | NBAT1 |
| 108 | SKA3 | CKS2 | PUS1 | FOSB | NDUFS5 | MIA2 | PIEZO1 |
| 109 | CENPJ | NLGN1 | THADA | TMPRSS4 | IFITM3 | ST3GAL4 | ECE1 |
| 110 | RAD18 | PLK4 | PABPC1 | PNPLA8 | C9orf16 | STK17B | ANO6 |
| 111 | ORC1 | BRCA2 | SLC12A8 | TMC5 | EEF1B2 | DAPK1 | DIP2B |
| 112 | ITGB3BP | PRC1 | RCL1 | IL1RN | TMSB10 | ETV6 | HAPLN3 |
| 113 | PCNA | G2E3 | TASP1 | CHKA | COX5B | LDLRAD4 | ADAM19 |
| 114 | SLFN13 | KIF24 | PPRC1 | CYSTM1 | UQCR11 | DOC2B | MAP1B |
| 115 | CDC25A | NEURL1B | NUBPL | AGR3 | C4orf48 | CHKA | CXCL1 |
| 116 | RFWD3 | NDE1 | METAP1D | PLEKHM1 | PRDX1 | CDON | HMGA2 |
| 117 | NDUFAF6 | UBE2S | UTP4 | PADI1 | PRDX5 | ZNF334 | SCD5 |
| 118 | BUB1B | SKA2 | TBC1D30 | MUC5AC | LYZ | FAM214A | PICALM |
| 119 | NCAPH | OIP5 | TAF4B | DENND2C | STEAP1B | BCAS1 | EDN1 |
| 120 | TOPBP1 | SPDL1 | NUDCD1 | FAM155A | C19orf33 | ARHGEF3 | TANC2 |
| 121 | GEN1 | MPHOSPH9 | ZNHIT6 | MPZL3 | NDUFB7 | AC019117.1 | MYO5B |
| 122 | SHCBP1 | ARHGAP33 | LARS | CRY1 | UQCRQ | CCPG1 | DSC2 |
| 123 | CHTF18 | SAPCD2 | ZNF121 | AC016831.7 | NDUFA4 | MPP7 | TGFB2 |
| 124 | PBX3 | TMPO | XPO6 | SLC6A20 | SERF2 | PAG1 | IL32 |
| 125 | TIMELESS | MXD3 | UBE3D | DSP | S100A11 | MX2 | DDX60L |
| 126 | NEMP1 | EZH2 | MRPS27 | GPCPD1 | NACA | GAN | PPP1R14C |
| 127 | MASTL | CDC25B | SNX5 | EHD4 | EEF1D | TSHZ2 | GLIS3 |
| 128 | LINC01572 | SCG5 | PKDCC | CAST | PPDPF | IGSF5 | TNFAIP2 |
| 129 | CDT1 | SMC2 | SLC7A6 | LRRFIP1 | PCOLCE2 | FMN1 | FERMT1 |
| 130 | C1orf112 | USP13 | HNRNPC | RASSF5 | ANAPC11 | MAML2 | FHOD3 |
| 131 | E2F8 | C2orf48 | MRRF | MYO5B | ALKBH7 | ANKMY2 | MIR181A2HG |
| 132 | NUP210 | KIF22 | EIF3J | ST14 | RPS26 | SMPD3 | MIR181A1HG |
| 133 | CCNE1 | TBC1D31 | ADAT2 | EVA1C | NDUFA2 | MBOAT1 | RND3 |
| 134 | CHAF1B | HP1BP3 | URB2 | ANXA2 | GSTP1 | MAP4K5 | HIVEP2 |
| 135 | PRKDC | POC1A | PMS1 | BLNK | UQCRH | ASS1 | RELB |
| 136 | MYO19 | HYLS1 | IPO5 | MRAP | TMSB4X | PRRX2 | NALCN |
| 137 | SASS6 | CNTRL | ZFAS1 | GTPBP1 | SRP14 | TPRG1 | ATP2B4 |
| 138 | PRIM1 | FBXO5 | ORC5 | DHRS9 | POLR2L | LGR6 | RBMS1 |
| 139 | NUSAP1 | CENPK | GTPBP4 | BCR | ARMC9 | PDK3 | BMP1 |
| 140 | RFC2 | DLEU2 | ACTR3B | AP4B1-AS1 | RPL5 | MCTP2 | KLF12 |
| 141 | CEP78 | CEP152 | MYC | PRAC1 | NDUFB11 | STRIP2 | MMP7 |
| 142 | GLYATL2 | RAD51AP1 | WDR75 | MEF2D | ZDHHC12 | ADGRF1 | CDKN2B |
| 143 | ZNF519 | TNFAIP8L1 | VWA8 | P3H2 | CST3 | PTAR1 | RPS6KA2 |
| 144 | TFDP1 | UBE2T | HEATR3 | MUC16 | SLPI | FGF3 | SKIL |
| 145 | SLFN11 | FAM72B | GNL3 | MAP2K3 | EEF2 | FNBP1 | TJP1 |
| 146 | RIBC2 | RAD21 | AHCY | KLF3 | COX7A2 | OAS3 | ADAMTS6 |
| 147 | DLEU2 | TTF2 | WRN | RAI1-AS1 | NHP2 | FAM155A | SMAD7 |
| 148 | RTTN | RTKN2 | PDSS1 | MALL | SOD1 | POLR2A | MYH9 |
| 149 | NDC80 | MZT1 | PITPNB | PLEKHG6 | TIMP1 | PCSK6 | NEGR1 |
| 150 | CDC7 | RCCD1 | EBPL | RLF | EIF2B5 | C4orf19 | MKL1 |
| 151 | MSH2 | CDK5RAP2 | DGKD | DIAPH1 | CHCHD10 | ADAM10 | INPP4B |
| 152 | SMC2 | FBXO43 | WDR35 | PDE4B | C12orf57 | TSPAN3 | AFAP1L2 |
| 153 | KIF4A | NCAPD3 | URI1 | COL17A1 | SPCS1 | SUSD6 | ACTN1 |
| 154 | DDX11 | BRD8 | GPR39 | SERPINB2 | SSR4 | PSMG3 | TMCC1 |
| 155 | CASP8AP2 | CEP70 | EIF4B | TSHZ2 | LDHB | DDX60 | ICAM1 |
| 156 | CCDC150 | ODF2 | KLHDC4 | SPAG9 | HNRNPA1 | GFPT1 | MYO10 |
| 157 | USP37 | H2AFX | ZC3H8 | TMEM178B | VIM | PRICKLE2 | KLF6 |
| 158 | C19orf48 | TBCD | HSPA9 | C3orf52 | MT1E | SWAP70 | NFATC2 |
| 159 | RFC5 | H2AFV | MRPS25 | VCL | PLEKHA7 | ICA1 | RND1 |
| 160 | PBK | SKA1 | PTCD3 | REEP3 | PPIA | LINC01558 | CAMK1D |
| 161 | FIGNL1 | CEP192 | SDAD1 | SERPINB5 | APRT | MTM1 | ZBTB46 |
| 162 | FANCL | FAM111A | TRNT1 | IFRD1 | NBEAL1 | TRIM14 | CAP1 |
| 163 | POLD3 | NDC1 | LARP4 | LRRC4 | TXN | CBX6 | MSN |
| 164 | PASK | LBR | CHCHD3 | ITGB1 | NPM1 | FKBP10 | CLEC16A |
| 165 | TMEM106C | HIST1H2BJ | SLC7A1 | WEE1 | TIMM13 | IFIT1 | EPHA4 |
| 166 | CENPM | C17orf53 | EIF3A | TMC7 | PFDN5 | FEZ2 | LBH |
| 167 | UBE2T | CHEK2 | LRP8 | RRP12 | EIF3F | SAMD12 | NFKB1 |
| 168 | PLK4 | FAM72D | QSOX2 | LIPE-AS1 | SEC61G | RP1 | PPARD |
| 169 | FOXM1 | LMNB2 | NPM1 | LMTK2 | PPIB | PTPN22 | CLDN1 |
| 170 | OXCT1 | ESCO2 | GTF2F2 | TNFRSF10B | CAV1 | SYTL2 | MAP3K9 |
| 171 | GPR137C | BARD1 | HNRNPR | BCL2L2 | PYCARD | PITX2 | HRH1 |
| 172 | CDK1 | CENPL | NFIA | ELL | DBI | PKIA | CPA6 |
| 173 | ECM2 | POLH | SRP72 | LINC00511 | NDUFAF3 | AGR2 | CDK6 |
| 174 | GINS2 | EXPH5 | CCT4 | MCF2L2 | COX6C | VRK2 | ITGB6 |
| 175 | PSMC3IP | SPC24 | TYW1 | MET | PRAP1 | SGPP2 | TNIP1 |
| 176 | FAM111A | KIAA0586 | SLC9B2 | SNRK | CHCHD3 | RND3 | AKAP12 |
| 177 | CEP295 | TUBB4B | GEMIN5 | HIPK1 | AMBRA1 | PDPR | COL6A1 |
| 178 | SMC6 | DTYMK | COA1 | ZDHHC11B | EIF3K | RBM47 | FBXL2 |
| 179 | MKI67 | ARHGAP11B | SIL1 | CD52 | GPX4 | ETNK1 | RTN4 |
| 180 | KIFC1 | VRK1 | USP10 | COL10A1 | AURKAIP1 | PAPSS2 | MIR4435-2HG |
| 181 | XRCC3 | CDC27 | UBAP2 | FAM129B | LSM3 | ARFGAP3 | ATP2C1 |
| 182 | C17orf53 | FANCI | GMDS | SLC2A1 | SUMO3 | TRIM31 | PKP1 |
| 183 | TMPO | RANGAP1 | SLC25A32 | SLC45A4 | CHMP3 | AGAP1 | ZEB1 |
| 184 | FANCC | CCDC77 | FILIP1L | RAB7A | POLR2K | BIRC3 | IQCJ-SCHIP1 |
| 185 | CCDC14 | DNMT3B | ANAPC7 | BCL2L1 | RAN | TEX9 | ADAMTS7 |
| 186 | NUP107 | CCDC88A | DDX18 | TPM3 | C19orf53 | SLC40A1 | RAB8B |
| 187 | PSIP1 | GINS1 | DNAJA3 | MYOG | IFI27 | GNAQ | BIRC3 |
| 188 | MIS18BP1 | EFCAB11 | USP36 | ADORA1 | SIK3 | AHR | C15orf53 |
| 189 | SAE1 | FANCD2 | FASTKD1 | PPP1CB | TMEM160 | STK39 | GNB1 |
| 190 | SPC24 | NAV2 | UTP6 | KLK10 | TIMM10 | SIDT1 | MAP4 |
| 191 | HAT1 | AC004158.1 | RPL5 | TSPAN3 | ERH | LNX1 | CLIC4 |
| 192 | CENPH | BUB1B-PAK6 | TANGO6 | ZBTB43 | YBX1 | MGAT4A | FLNB |
| 193 | RAD51B | NEMP1 | NSUN2 | ABTB2 | LGALS1 | TNFAIP2 | BTG1 |
| 194 | LIN52 | TDP1 | EIF2S1 | TES | UBL5 | GALNT5 | SERPINB7 |
| 195 | TACC3 | CENPO | GMPS | COBL | TSPO | STRN | NFKBIA |
| 196 | ZWILCH | CENPJ | RABEPK | GPS2 | CHCHD5 | PKN2 | BICC1 |
| 197 | CCDC15 | TRAIP | DHX34 | F3 | SNRPF | NUMB | NEK7 |
| 198 | CAMK4 | FANCM | CHCHD6 | LITAF | C19orf70 | CD47 | MMP14 |
| 199 | CENPO | WEE1 | SRSF7 | NEAT1 | ACTB | ANXA4 | JUP |
| 200 | RIF1 | KMT5A | GSPT1 | PACSIN2 | PGLS | PTPRZ1 | PXN |

Malignant lineage programs

| Gene | Acinar- | Classical- | | | | Neuroendocrine- | Neural-like |
|---|---|---|---|---|---|---|---|
| Rank | like | like | Basaloid | Squamoid | Mesenchymal | like | progenitor |
| 1 | SYCN | PTH2R | IGF2 | KRT13 | COL1A2 | MYL7 | KCNJ16 |
| 2 | CLPS | SULT1C2 | CST6 | PSCA | SPARC | LINC00363 | ZBTB16 |
| 3 | AMY2A | ANXA10 | CRYAB | KRT16 | COL3A1 | GCK | CTNND2 |
| 4 | PLA2G1B | HEPH | CST4 | AC079466.1 | LUM | KCNB2 | PDE3A |
| 5 | CTRB2 | WDR72 | FBXO2 | DHRS9 | AEBP1 | VWA5B2 | PDGFD |
| 6 | CELA3A | FMO5 | CHPF | MUC5AC | COL6A3 | HS6ST3 | CNTN4 |
| 7 | CTRC | SULT1B1 | LGALS1 | A2ML1 | COL1A1 | CACNA1B | CFTR |
| 8 | PNLIP | SYTL2 | ALDOA | IFITM10 | BGN | TMEM196 | FLRT2 |
| 9 | CPA1 | BTNL8 | MT1E | FAM83A | SFRP2 | RLN1 | ADCY5 |
| 10 | CTRB1 | PLAC8 | ISG15 | AC019117.2 | MMP11 | GALR1 | C6 |
| 11 | CPA2 | STXBP6 | CCDC85B | KCP | ACTA2 | IRX2 | CRISP3 |
| 12 | CELA3B | FER1L6 | LY6K | EPS8L1 | FN1 | KCNAB1-AS1 | RALYL |
| 13 | CPB1 | TM4SF20 | KRTAP2-3 | CSNK1E | THBS2 | RTN1 | NR1H4 |
| 14 | GSTA2 | ETNK1 | MT2A | PADI1 | VIM | RFX6 | BCL2 |
| 15 | PNLIPRP1 | KCNJ3 | CKAP4 | PLXNB2 | POSTN | MAGEB17 | ESRRG |
| 16 | CELA2B | CPS1 | PRNP | CDK5RAP3 | EMILIN1 | SLC30A8 | SLC4A4 |
| 17 | GP2 | CASR | IFI27 | NBEAL2 | DCN | ST18 | CSMD2 |
| 18 | AQP8 | THRB | DKK3 | USP39 | IGFBP5 | KCNK16 | RGS17 |
| 19 | REG1A | PIP5K1B | C9orf16 | SLC16A3 | THY1 | S100Z | CRP |
| 20 | CEL | REG4 | GJA1 | TRIM29 | DES | TAT | SLC17A4 |
| 21 | MT1G | BCAS1 | IFI6 | ALS2CL | COL6A2 | NCAM1 | RELN |
| 22 | RBPJL | NR3C2 | CRIP1 | ITGB4 | MGP | NKX2-2 | PAH |
| 23 | PRSS1 | SIPA1L2 | POLR2L | PTGDS | CTHRC1 | CAMK2B | PKHD1 |
| 24 | LPL | CLDN18 | THBS1 | APOL1 | TAGLN | XKR4 | LINC01320 |
| 25 | APOD | PELI2 | LGALS7 | KAT2A | CALD1 | PCSK2 | ACSM3 |
| 26 | ENTHD1 | TMEM45B | TNNC2 | MROH6 | MMP2 | SH3GL2 | DSCAML1 |
| 27 | RARRES2 | SLC3A1 | PTMS | SGSM3 | CARMN | ATRNL1 | AR |
| 28 | SOD3 | CBLB | ROMO1 | KRT19 | ISLR | MIR7-3HG | ZNF208 |
| 29 | KLK1 | PRKG1 | IFI27L2 | PIM3 | PDGFRB | ABCC8 | TTLL7 |
| 30 | PRSS2 | GPC5 | CD81 | SNHG25 | MYL9 | TRPM3 | SOX6 |
| 31 | RPL18A | ARHGEF38 | TUBB | RAPGEFL1 | CTGF | NRXN1 | SPP1 |
| 32 | REG1B | SLC41A2 | LY6E | SMCR5 | COL5A1 | AMPH | ASXL3 |
| 33 | RNASE1 | ATP10B | C12orf57 | DDX17 | IGKC | CACNB2 | POU6F2 |
| 34 | FXYD2 | NR5A2 | PSAP | TFCP2L1 | ACTG2 | GNAO1 | DZIP1 |
| 35 | GAMT | PLD1 | PDLIM4 | MUC5B | SPARCL1 | GAD2 | ITIH5 |
| 36 | MIR217HG | CYP3A5 | C19orf53 | CIRBP | LGALS1 | DSCAM | PRKG1 |
| 37 | ZMAT5 | TSPAN8 | CTSZ | MRO | CCDC80 | CPE | IL1R1 |
| 38 | PNPLA4 | RAB27B | SNRPD2 | AMN | GREM1 | KCNT1 | SEMA3E |
| 39 | CUZD1 | LRRC66 | GPS2 | KRT7 | MMP9 | CALY | GUCY1A2 |
| 40 | CCL14 | MCU | OST4 | UBALD2 | C11orf96 | CACNA1A | AC092535.3 |
| 41 | EFCAB3 | SLC40A1 | PRDX1 | ECM1 | HTRA1 | FGA | RCAN2 |
| 42 | SLC47A1 | XRCC4 | NPC2 | IFT27 | IGHA1 | LRRTM4 | SLC3A1 |
| 43 | AMY2B | CAPN8 | VIM | CLIC3 | HTRA3 | DDC | MAN1A1 |
| 44 | PDIA2 | ABHD2 | TRMT112 | ARRDC2 | CYR61 | MTMR7 | WDR72 |
| 45 | REG3A | ANPEP | MZT2B | ZC3H11A | COMP | TMEM132D | ACSS3 |
| 46 | DBET | MITF | SCAND1 | DUSP1 | TIMP1 | UNC79 | ONECUT1 |
| 47 | CELA2A | PCSK5 | MMP2 | INF2 | COL15A1 | TMOD1 | NRP1 |
| 48 | REG3G | DUOXA2 | CD59 | DNAJB13 | FLNA | INHBA-AS1 | AKAP7 |
| 49 | RPL9 | GCNT3 | MT1A | ENO2 | TCF4 | GNG4 | LDLRAD4 |
| 50 | GATM | IQGAP2 | CFL1 | LYNX1 | A2M | PCBP3 | AC012593.1 |
| 51 | RNF181 | ADGRG7 | SEC61G | PDE4C | FBLN1 | RAB3C | CALN1 |
| 52 | RPS28 | KCNK1 | TMSB10 | MSLN | DNM3OS | KIF1A | UGT2B15 |
| 53 | STEAP1B | CCDC68 | BANF1 | ALDH3B1 | C1R | PTPRT | AGBL4 |
| 54 | RPS17 | MYO7B | SOSTDC1 | UNC5B-AS1 | TUBA1A | SAMD3 | GLIS3 |
| 55 | C12orf57 | RHOBTB1 | VAT1 | TRABD | APOD | UNC13A | TRPV6 |
| 56 | ATRAID | DUOX2 | MRPS12 | LIF | EGFL7 | SCGN | ABCB1 |
| 57 | MTRNR2L8 | KIAA1211 | PPDPF | GAPDH | PDLIM3 | ABCB1 | PLXDC2 |
| 58 | ISCU | PIK3C2G | NPB | PLEC | IGLC2 | IQSEC3 | NLGN4Y |
| 59 | PRDX4 | XKR9 | TIMM8B | ACADVL | IGLC3 | GRIA2 | NEK10 |
| 60 | ZNF615 | FSIP2 | UBA52 | KRT17 | LAMA2 | HEPACAM2 | TACC1 |
| 61 | SPINK1 | SLC19A3 | NBL1 | ADAM15 | IGHGP | LINC01428 | HOMER2 |
| 62 | SLC22A31 | ANTXR2 | CENPB | SYT8 | FBXL7 | KIF5C | SNAP25 |
| 63 | SLC1A2 | HSD17B2 | DGCR6 | S100A6 | INHBA | JAKMIP2 | SCTR |
| 64 | PEBP1 | SHROOM3 | GADD45GIP1 | CPSF1 | GGT5 | SGCD | SLC2A2 |
| 65 | CDIPT | FAM177B | COX5B | AC159540.1 | RARRES2 | G6PC2 | MIR99AHG |
| 66 | PCOLCE2 | HSD17B11 | DYNLL1 | FSTL3 | PDGFRA | SLC29A4 | TRABD2B |
| 67 | ZNF90 | PDCD4 | ANAPC11 | APOL2 | EDNRA | SNTG1 | NR5A2 |
| 68 | SERPINI2 | A1CF | GPX3 | GUK1 | COL6A1 | FBXO43 | FAM135B |
| 69 | RPS12 | CAMK2D | LINC01615 | VILL | GNG11 | RIMBP2 | FGG |
| 70 | AC072062.1 | RASSF6 | PPIA | SPRR1B | CNN1 | NCAM1-AS1 | DCDC2 |
| 71 | ZDHHC24 | PRSS12 | CCND1 | PLAT | IGFBP7 | RIMS2 | SYNE1 |
| 72 | XAB2 | HNF4G | RABAC1 | NAPRT | MFAP4 | NOL4 | CRISP2 |
| 73 | CLU | FAM135A | COX6B1 | ZNF692 | ADAMTS2 | SNAP91 | SEMA5A |
| 74 | RPL34 | SULT1E1 | CTGF | ERCC5 | CD93 | PPM1E | BICC1 |
| 75 | MCUR1 | PAK1 | RPS17 | DDX5 | FSTL1 | CELF4 | TNS1 |
| 76 | EPHX1 | ATP7B | TNFRSF12A | GPR153 | PXDN | SLC16A12 | CHST9 |
| 77 | RPS3A | LGR4 | SH3BGRL3 | SCEL | SPON2 | LINC01164 | NCAM1 |
| 78 | UBA52 | SLC4A4 | RPL35 | MKNK2 | SFRP4 | MYO3A | RORA |
| 79 | RPL21 | VSIG1 | PRDX5 | LAMB2 | COL4A2 | NECAB2 | ADAMTS9-AS2 |
| 80 | IGFN1 | CDH17 | HSBP1 | MYO16-AS1 | SERPINF1 | ACOT12 | APCS |
| 81 | LINC01450 | POF1B | TUBA1B | TP53I3 | GPNMB | LINC00355 | MUM1L1 |
| 82 | MZT2B | HPGD | TMSB4X | FOS | COL5A2 | GHRHR | SETBP1 |
| 83 | RPS18 | ITGA1 | NUPR1 | COL17A1 | CD81 | DENND2A | NRCAM |
| 84 | RPS6 | MGAT5 | RHOC | PRSS8 | TIMP3 | ANKRD30B | CFAP221 |
| 85 | RPL3 | CAPN9 | TMED9 | WSB1 | SLC11A1 | ADGRV1 | ATP13A4 |
| 86 | RPL7A | PTPRR | RARRES3 | DEFB1 | RGS1 | ZNF831 | LIN7A |
| 87 | RPL11 | TRIM2 | GAPDH | GRIK2 | APOE | RGS22 | TTC28 |
| 88 | IMPACT | FRY | S100A2 | LAMA5 | ID3 | PLPPR1 | STXBP6 |
| 89 | RPS4X | REPS2 | FJX1 | TSPO | MT-CO3 | LINC01554 | KCTD16 |
| 90 | RPL32 | TFPI | MZT2A | MOGAT1 | GCG | LINC01060 | NR2F2-AS1 |
| 91 | RPS15 | AC019117.1 | UQCRQ | TIMP2 | LRRC32 | ELMO1 | DOCK8 |
| 92 | TMED5 | LIMA1 | NFE2L1 | COL6A1 | IGFBP4 | GABBR2 | LRRK2 |
| 93 | RPS8 | BLNK | C19orf33 | TMEM259 | MT-CO2 | ADCY1 | RERG |
| 94 | EEF1A1 | HMGCS2 | ATP6V1G1 | MMP28 | PLVAP | SRRM3 | AC018742.1 |
| 95 | NPHS1 | MRAP2 | RPL8 | B3GALT5 | ITGA11 | KL | PCDH9 |
| 96 | GCDH | SPINK1 | SDC1 | FGF19 | LMOD1 | LINC00907 | PTCHD4 |
| 97 | PPP1R27 | PLA2G10 | HLA-DQB1 | PNPLA3 | MT-ND3 | ERO1B | ATP10A |
| 98 | TMED3 | TSPAN3 | SFN | SCNN1A | CLEC11A | KBTBD8 | WNK2 |
| 99 | OR10G4 | RASEF | FAU | GLUL | LAPTM5 | KCNH6 | DPYD |
| 100 | MYRIP | LRRC31 | CALR | PLEKHH3 | MT-CO1 | BRINP1 | AC019117.1 |
| 101 | FBXO31 | FMN1 | TMED2 | DDIT4 | GYPC | CACNA1C | SNCAIP |
| 102 | RPL6 | MUC3A | FTH1 | TTLL12 | CRISPLD2 | POU6F2 | LIMCH1 |
| 103 | RPL13A | NPNT | NDUFB10 | SYTL1 | NOX4 | OR2M4 | ANXA4 |
| 104 | RPL14 | ABHD3 | MLLT11 | KCNJ6 | ELN | GRM7-AS3 | BMPR1B |
| 105 | RPL35A | BACE2 | EFCAB3 | AC002066.1 | PODN | EML5 | KCNMA1 |
| 106 | RPL41 | CCSER1 | RPL38 | STRA6 | C7 | ELAVL4 | PTP4A1 |
| 107 | RPS2 | MAP2 | TUBA1A | SMIM5 | BOC | LINC01446 | DTNA |
| 108 | NUTF2 | ARHGAP42 | B3GALT6 | DUS1L | NTM | SNAP25 | NBEA |
| 109 | RPL7 | MECOM | FSTL1 | CAPN2 | DPYSL3 | GRM8 | TTN |
| 110 | TXNRD1 | DEPTOR | GSTP1 | MST1R | MXRA8 | MAGI2 | DLG2 |
| 111 | RPS16 | UGT2A3 | RNF181 | SPRR3 | LIMS2 | UNC80 | PTPRM |
| 112 | RPS25 | HNF4A | RPL28 | TNFAIP2 | TBX2 | CNTN4 | SCN9A |
| 113 | UQCRB | PPARG | CHCHD2 | LAMB3 | RUNX1T1 | HMGCLL1 | TMEM132C |
| 114 | RPL29 | B4GALT4 | MRPL51 | SCO2 | IFFO1 | AFF3 | HIF1A |
| 115 | RPS27 | PRSS3 | RPS13 | C19orf33 | APOO | ZNF732 | KHDRBS2 |
| 116 | RPS7 | GMDS | TMBIM6 | CSTB | SGIP1 | CA10 | SLC16A7 |
| 117 | RPLP0 | RAB3B | MYL6 | ARHGAP8 | TTR | LINC00608 | AJAP1 |
| 118 | RPS13 | POC1B | UQCR10 | SKIV2L | HIC1 | FRRS1L | KIF12 |
| 119 | RPS27A | STX7 | RPS19BP1 | CIB1 | COL4A1 | TMEM200A | SEMA6A |
| 120 | ACADL | TP53INP1 | FTL | QTRT1 | FTL | CYP46A1 | GRM8 |
| 121 | TNFRSF9 | SYT16 | NDUFS5 | KIFC2 | CD248 | GALNT16 | LRAT |
| 122 | FAU | CLINT1 | POLR2K | RGL2 | RPL15 | FGF14 | TRIM5 |
| 123 | RPS21 | ZSWIM6 | IL32 | RGL3 | TGFBI | KCNMA1 | NFIB |
| 124 | RPL35 | DNM2 | SAA1 | ST3GAL4 | PCOLCE | SLC35F4 | PDE7A |
| 125 | SLC30A2 | LPIN1 | RCN1 | TXNIP | MT-ATP6 | STXBP5L | ONECUT2 |
| 126 | RPL15 | TCF7L2 | COPRS | TPI1 | ANXA6 | GPR137C | PRICKLE2 |
| 127 | ANKRD62 | STK39 | RBP1 | NISCH | MFAP2 | DOCK3 | CDH6 |
| 128 | BDNF-AS | ADAM23 | KRT17 | IRF8 | COL11A1 | ACTL6B | AC124312.1 |
| 129 | RNF19B | SPIRE2 | TXN | STXBP2 | SERPING1 | USH2A | MAPK10 |
| 130 | RPS14 | LYPD6B | SLC39A4 | CYTH2 | MYH11 | CD226 | RBPMS |
| 131 | P4HB | ERN2 | CCDC167 | ATG4B | RGS16 | GPR162 | APCDD1 |
| 132 | ERO1B | CD164 | PERP | LINGO1 | ITGAX | CACNA2D3 | CES1 |
| 133 | PCYT2 | SH3RF1 | TIMP3 | COL9A2 | CSRP1 | SLC38A4 | LINC01266 |
| 134 | RPS24 | PHGR1 | AURKAIP1 | MEGF6 | DKK3 | SERPINI2 | SERPINA6 |
| 135 | KIF1A | GAREM1 | MAF1 | C12orf56 | PSAP | MSI1 | ADGRL2 |
| 136 | RPL28 | USP53 | RPS27A | ENGASE | RPL3 | CACNA1H | LINC00671 |
| 137 | RPL24 | ABI3BP | FBN1 | SMTN | NUPR1 | DMGDH | TENM3 |
| 138 | RPL27A | DHRS9 | P4HB | D2HGDH | FMNL3 | CLSTN2 | KCNJ15 |
| 139 | RNF212 | PDZD3 | ZNF593 | TNNI2 | TMEM158 | TRIM9 | MEIS2 |
| 140 | SNTG2 | UGCG | RPL27 | PKM | MMP19 | FMN2 | FIGN |
| 141 | MT-CO2 | OCLN | RPLP2 | MAP3K6 | ADAMTS12 | SLC12A5 | GABRB3 |
| 142 | PTGER3 | ATP6V0A1 | PDZRN3-AS1 | PDXK | MRC2 | GCG | SDK1 |
| 143 | ARMC9 | PRKACB | C4orf48 | BIK | FBN1 | LINC01214 | KCNT2 |
| 144 | FAM129A | SLC5A1 | IGFBP6 | NUDT14 | PPP1R14A | SH2D3C | ZNF503-AS1 |
| 145 | RPLP2 | PPP1R12B | SOD1 | PLAC8 | HAND2 | FAM187B | CFH |
| 146 | RPLP1 | LGALS4 | RPS2 | CTDSP1 | GRASP | AC113404.1 | ARHGAP44 |
| 147 | RPL5 | FBXO34 | ZNHIT2 | EPS8L2 | MT-ND1 | LINC01474 | ANKS1B |
| 148 | RPL31 | FER1L6-AS2 | LGALS3BP | HDAC10 | CXCL12 | VGF | THSD4 |
| 149 | RPL8 | GULP1 | SLC7A5 | MIR210HG | RPLP1 | KIAA1324 | NR3C2 |
| 150 | EEF2 | MAP7 | B4GALNT1 | LINC01060 | MT-ND4 | SYT14 | ADARB2 |
| 151 | SIAH2 | NELL2 | SERF2 | RIOK3 | LAMP5 | MEG3 | NRXN3 |
| 152 | HPN | FGD4 | CHCHD10 | ITGA3 | ITGA5 | PDZRN3 | TOX |
| 153 | RPSA | PRKCA | HSPA5 | AQP5 | ZEB2 | PTPRN | NFIA |
| 154 | EGF | SEMA6D | EXOSC4 | LZTR1 | COL18A1 | CELF3 | ANPEP |
| 155 | MT-CO1 | ACE2 | CHCHD5 | RECQL5 | CD34 | DPP6 | GRB10 |
| 156 | TTN | GDA | CDA | MGMT | KIAA1755 | PHF21B | TUSC3 |
| 157 | RADIL | SIDT1 | RPS19 | MICALL2 | C1S | CPLX2 | HABP2 |
| 158 | ZNF98 | CAPN5 | H2AFJ | MRPL55 | EVA1B | ADAMTSL1 | DACH1 |
| 159 | CCDC54 | TOX3 | ATP6V1F | ANGPTL4 | HSPB6 | FSTL4 | MPP6 |
| 160 | APOO | PDE3B | TRIM54 | RHPN1 | RAMP2 | NRCAM | CAMK1D |
| 161 | MAMDC2 | ACSS2 | RPL36 | RFNG | BNC2 | INMT | SLC1A1 |
| 162 | RPL18 | PDLIM5 | LDHB | SH3TC1 | ADAM33 | PLCXD3 | SERPING1 |
| 163 | BCAP31 | MLPH | TOMM7 | FDPS | OLFML2B | KCNK17 | MEF2C |
| 164 | RPS11 | ONECUT2 | RPS25 | FUT11 | PHLDB1 | KCNJ6 | MUC5B |
| 165 | LINC01625 | COBLL1 | PEBP1 | ARHGEF16 | MCAM | GADD45G | FHIT |
| 166 | RPS3 | CHPT1 | RPL41 | TRMU | CPE | TTR | SLC5A1 |
| 167 | MKNK1 | MTUS1 | CRABP2 | SLC22A3 | ADAMTS4 | MMP16 | PDGFC |
| 168 | MT-ND4 | UNC5CL | FSTL3 | RPL13 | SLIT3 | CACNA2D2 | ASRGL1 |
| 169 | EEF1D | TNIK | POPDC3 | MLLT6 | SPP1 | ABCC9 | SULT1C4 |
| 170 | RPL36 | EPS8 | YWHAB | RASSF7 | ELMO1 | MPP2 | CACNA1H |
| 171 | OR8D4 | PLS1 | S100A11 | DEPDC5 | SRGN | KCNH7 | NREP |
| 172 | MT-ND3 | LRRFIP2 | COA3 | PHLDA3 | WNT5A | ENPP2 | DSEL |
| 173 | RPS20 | FRRS1 | VAMP8 | TJP2 | WISP1 | SPTBN4 | SLC4A7 |
| 174 | XPO5 | ARL14 | COX6C | ARHGAP27 | IGHG3 | SYT7 | REG1A |
| 175 | NUPR1 | DAPK2 | TACSTD2 | H2AFJ | C1QB | ODF2-AS1 | MLLT3 |
| 176 | RPL13 | C2CD5 | C1orf122 | AC098828.2 | MEDAG | DDX25 | TDRP |
| 177 | RPL4 | SLC44A1 | HLA-C | FBXW5 | TMEM204 | CRYBA2 | DLGAP1 |
| 178 | TNIP3 | CYP2C9 | KRT8 | A4GALT | NRXN2 | RASGRF1 | ST8SIA3 |
| 179 | SLC16A10 | MGAM2 | PODNL1 | LINC01322 | MT-CYB | ZNF730 | FXYD2 |
| 180 | DHFR | NR1I2 | EIF1 | FLNB | IL1R1 | FAM163A | EPB41L4A |
| 181 | DUPD1 | MAP4K3 | RPL35A | CSPG4 | ZBTB16 | PRUNE2 | IQCA1 |
| 182 | MT-CO3 | WLS | NDUFB9 | FXYD3 | HLA-DRA | CRHBP | PRKCE |
| 183 | PAK3 | FRK | UQCRB | PPL | PIK3R5 | AGBL4 | MPP7 |
| 184 | NR5A2 | FUT8 | AP2S1 | ABCA7 | LMCD1 | NPAS3 | NRG1 |
| 185 | CD79B | SH3BGRL2 | CYSRT1 | CDK10 | SLC8A1 | NRXN3 | ITPR2 |
| 186 | ISY1 | MAPRE2 | RPL19 | TRMT2A | COL16A1 | SDK1 | ZNF667 |
| 187 | PSAP | RNF128 | NXT1 | LENG8 | NBL1 | ZNF423 | AUTS2 |
| 188 | C2CD4B | TFF1 | SNCG | DNAJB2 | RPS23 | EMID1 | LRP1B |
| 189 | BRSK2 | TMEM163 | MARCKS | CD151 | SAMD11 | ARX | BCO2 |
| 190 | FLG | FBXW11 | IFITM3 | P4HB | F2R | MAP1B | PBX1 |
| 191 | RPS23 | NRG4 | FAM210B | PIEZO1 | SLC2A3 | DCLK2 | RASSF8 |
| 192 | NACA | HECTD1 | ISOC2 | SHROOM1 | CYP1B1 | VLDLR-AS1 | HYDIN |
| 193 | AOX1 | SCIN | UQCRHL | TRIP10 | SFTPC | APOLD1 | PRKD1 |
| 194 | SLC39A5 | UGT2B15 | BAD | AMT | TMEM119 | ZNF385D | ZSCAN18 |
| 195 | ZBTB16 | MAN1A1 | FAM83H | FKBP2 | MAFB | KCNB1 | KLKB1 |
| 196 | MT-ND1 | NCOA2 | PGAM1 | EPHA2 | NNMT | DYNC1I1 | ZNF676 |
| 197 | CD63 | SLC22A23 | CREB3 | HDAC7 | VASH1 | LOXHD1 | PBX3 |
| 198 | ITLN2 | AGBL1 | BOLA3 | TRPV2 | NHSL2 | GNAT3 | CEP112 |
| 199 | MT-ATP6 | SYBU | ADAD2 | KCNN4 | OLFML3 | ZBTB16 | CYS1 |
| 200 | SLC7A2 | TNFRSF11A | NNMT | FKBP5 | ZNF521 | GPR63 | GALNT18 |

Fibroblast programs

| | Adhesive | Immunomodulatory | Myofibroblastic | Neurotropic |
|---|---|---|---|---|
| 1 | NFATC2 | SLC22A3 | ADAMTS12 | SCN7A |
| 2 | EMP1 | XKR4 | CASC15 | NFIA |
| 3 | MIR222HG | ANKRD29 | POSTN | C7 |
| 4 | SAMD4A | SLCO2B1 | NTM | PID1 |
| 5 | LMNA | LAMA3 | LINC01429 | C1orf21 |
| 6 | GPRC5A | ABCC3 | NREP | MAMDC2 |
| 7 | MMP19 | LAMC2 | PDGFC | CLMN |
| 8 | MEDAG | GRIN2B | LEF1 | PREX2 |
| 9 | NFATC1 | RBM47 | NUAK1 | MTUS1 |
| 10 | TSC22D2 | NOL4 | COL1A1 | ADAMTS9-AS2 |
| 11 | LRRFIP1 | CP | KIF26B | KCNIP1 |
| 12 | RFX2 | KEL | NOX4 | LAMA2 |
| 13 | PFKP | ZNF804B | FN1 | EBF1 |
| 14 | PTPRJ | TNC | SULF1 | ABCA6 |
| 15 | ANKRD28 | ACTB | COL1A2 | NID1 |
| 16 | CAV1 | TMEM108 | WNT5A | EPHA3 |
| 17 | TEX26-AS1 | TMEM178B | COL3A1 | IL1RAPL1 |
| 18 | CDH2 | CCL21 | COL11A1 | TMEM132C |
| 19 | ANXA2 | ABCB11 | CDH11 | SPTBN1 |
| 20 | CTNNAL1 | SLCO2A1 | NKD1 | ADAMTSL3 |
| 21 | SLC19A2 | IL15 | DOCK4 | NEGR1 |
| 22 | CRY1 | FDCSP | PLPP4 | AC016831.7 |
| 23 | CNN1 | MUSK | MMP11 | SLC9A9 |
| 24 | SYN3 | PLA2G4C | ADAMTS14 | MIR99AHG |
| 25 | ANXA5 | ATP8A1 | ADAMTS6 | ZBTB20 |
| 26 | TES | ADGRL3 | FAP | SRPX |
| 27 | LHFPL2 | LIFR | RUNX2 | ABCA8 |
| 28 | LMCD1 | NPY1R | RUNX1 | TGFBR3 |
| 29 | ERRFI1 | ARHGAP15 | MGAT5 | ABCA10 |
| 30 | UGP2 | CTSS | SNTB1 | PTEN |
| 31 | LMCD1-AS1 | RASGEF1B | KIAA1549L | ZBTB16 |
| 32 | IQCJ-SCHIP1 | BIRC3 | CTHRC1 | RHOBTB3 |
| 33 | ACSL4 | NRG2 | LINC00578 | SLIT2 |
| 34 | ZSWIM6 | JUN | RNF144A | PDK4 |
| 35 | DDAH1 | LPAR1 | ENC1 | FREM1 |
| 36 | PTPN1 | EXOC3L4 | SYTL2 | SOX6 |
| 37 | ABL2 | PTPRF | ITGA1 | CACNA1D |
| 38 | ESYT2 | CR2 | DCBLD1 | ABI3BP |
| 39 | GFPT2 | CHL1 | COL10A1 | HMGCLL1 |
| 40 | ATP13A3 | EGR1 | CALD1 | AOX1 |
| 41 | BAIAP2 | ANO9 | CARMN | MAPK10 |
| 42 | GLIS3 | SLCO1A2 | CHST11 | SSH2 |
| 43 | ERCC1 | ZFP36L2 | PDZD2 | KAZN |
| 44 | CD44 | OSMR | ANTXR1 | ARHGAP10 |
| 45 | ENAH | EDNRB | GREM1 | AFF3 |
| 46 | SERPINE1 | PTMA | INHBA | ARHGAP6 |
| 47 | CLIC4 | PLD5 | NPR3 | ABLIM1 |
| 48 | ATP10A | EHBP1L1 | GRIP1 | PTPRG |
| 49 | FNIP2 | TIMM23B | SLC6A6 | ADGRD1 |
| 50 | MYOF | TRAF1 | FBXO32 | SPARCL1 |
| 51 | NEDD9 | TAGLN | FGD6 | FKBP5 |
| 52 | FOSL1 | RASGEF1A | SALL4 | ABCA9 |
| 53 | RTN4 | EEF1A1 | KCND2 | ANKS1B |
| 54 | COBL | CACNA2D3 | ITGA11 | COL21A1 |
| 55 | MYH10 | ADRA1A | MIR181A1HG | FRMD3 |
| 56 | FOSB | S1PR3 | ACTA2 | IMMP2L |
| 57 | KDM6B | IER3 | LAMA4 | CELF2 |
| 58 | CAPN2 | PLCXD3 | APBB2 | ADD3 |
| 59 | ANXA1 | SLC26A7 | EDNRA | CCNH |
| 60 | YWHAZ | IRF8 | FUT8 | HAND2-AS1 |
| 61 | RGCC | NFAM1 | BICD1 | DSCAML1 |
| 62 | EGFR | PDE4B | MBOAT2 | TFPI |
| 63 | HIF1A | SORL1 | PALM2-AKAP2 | NR2F2-AS1 |
| 64 | SH3RF1 | ACHE | SUGCT | BOC |
| 65 | ELL2 | SLC26A3 | HIP1 | ADGRB3 |
| 66 | KLF6 | TPM4 | VSNL1 | PDE1A |
| 67 | WEE1 | CDH1 | ENTPD1 | MKLN1 |
| 68 | S100A10 | CTSH | SGIP1 | NFIB |
| 69 | P4HA3 | PAPLN | EEPD1 | PBX1 |
| 70 | HOMER1 | SDK1 | KANK4 | FBLN5 |
| 71 | TRIB1 | DAPK2 | FRMD5 | CPED1 |
| 72 | ADAM12 | ACTG1 | PPFIBP1 | HIF3 A |
| 73 | ITGA5 | DEPTOR | FOXP1 | PIK3R1 |
| 74 | SLC7A1 | CYSLTR2 | ADAM19 | TENM2 |
| 75 | KCNMA1 | DTNA | SIPA1L1 | COL4A4 |
| 76 | TUBB6 | COL27A1 | FARP1 | SESN3 |
| 77 | HRH1 | CXCL12 | PTK7 | ITPR1 |
| 78 | GEM | TNFAIP2 | NHSL1 | DLG2 |
| 79 | GPR176 | NR4A1 | VCAN | FBLN1 |
| 80 | PCGF5 | LINC01197 | HMGA2 | SSBP2 |
| 81 | MICAL2 | CR1 | EPSTI1 | GPHN |
| 82 | PER2 | CSF2RB | CDK6 | ADAMTS3 |
| 83 | ST6GALNAC5 | VCAM1 | SPATS2L | SAMHD1 |
| 84 | DOK5 | TMSB4X | PALLD | KCTD3 |
| 85 | LOX | LMF1 | STAMBPL1 | LINC01088 |
| 86 | COL12A1 | OCA2 | RASGRF2 | NEURL1B |
| 87 | TIMP3 | RPS9 | MYH9 | RUNX1T1 |
| 88 | ACTN4 | TNFRSF1B | ARHGAP31 | GPC5 |
| 89 | FLNB | THBS1 | CDKL5 | SOX5 |
| 90 | CRIM1 | LDLR | TPM1 | ADGRL2 |
| 91 | PMEPA1 | PTPRT | ATXN1 | AFF1 |
| 92 | EFHD2 | MYO16 | PTPRE | NOVA1 |
| 93 | S100A6 | EBF3 | ZEB1 | PARD3 |
| 94 | PXDC1 | TLR1 | FAM168A | GFRA1 |
| 95 | MYO1B | C1RL-AS1 | ST6GAL2 | CCND3 |
| 96 | TAOK3 | FOS | COL5A1 | FAM13A |
| 97 | MLF1 | SERPINB9 | FNDC1 | MFSD6 |
| 98 | UAP1 | COL23A1 | PLXNC1 | RGL1 |
| 99 | CORO1C | GNA14 | EIF4G3 | SETBP1 |
| 100 | TIPARP | PKP2 | ANO1 | GHR |
| 101 | ITGB1 | FTH1 | LRIG3 | DYNC1I1 |
| 102 | DENND5A | SAT1 | TCF4 | CCDC102B |
| 103 | MEF2A | NCEH1 | HOXB3 | SPATA6 |
| 104 | RNF149 | TNFAIP3 | APBA2 | NSF |
| 105 | MKL1 | JUND | GULP1 | DCN |
| 106 | MYO9B | TPT1 | HECW1 | LDLRAD3 |
| 107 | EXT1 | KIAA1671 | TLN2 | DCLK1 |
| 108 | GLUD1 | PNISR | SPIN1 | RNF13 |
| 109 | FNDC3B | PCOLCE2 | IRS1 | RORA |
| 110 | GADD45B | FGF7 | SPON2 | NLGN1 |
| 111 | NUP153 | ITIH5 | NXN | CECR2 |
| 112 | HMGA1 | UBC | TSC22D1 | FOXO3 |
| 113 | FGFR1 | HDAC9 | TENM4 | COL4A1 |
| 114 | FHL2 | CYR61 | GRIK2 | UTRN |
| 115 | ARC | ADAMTSL1 | NPAS2 | PIAS1 |
| 116 | CBLB | GRIA4 | STX7 | COL4A3 |
| 117 | MBNL2 | GARNL3 | BCAT1 | CACNB2 |
| 118 | ACTN1 | IL4R | PRICKLE1 | COL4A2 |
| 119 | FAM155A | SPNS2 | ZNF521 | FIGN |
| 120 | FSIP1 | NBEAL2 | ZNF532 | FMNL2 |
| 121 | GATAD2A | ZFP36 | KLHL2 | SOBP |
| 122 | CREB5 | PNRC1 | ITGB5 | EGFR |
| 123 | SIK3 | PARP14 | TNFRSF19 | TGFBR2 |
| 124 | KALRN | TXNIP | ARMC9 | FAM102B |
| 125 | CDK17 | STRIP2 | TNS3 | DPYD |
| 126 | ITPKC | SVEP1 | GFPT1 | FAM135A |
| 127 | PDLIM5 | FADS1 | MRVI1 | PPP1CB |
| 128 | PSME4 | PLXNA4 | WNK1 | IRAK3 |
| 129 | NCS1 | SLC2A3 | MANBA | TMEM144 |
| 130 | MBNL1 | LINGO1 | TBL1XR1 | MGST1 |
| 131 | CAMK1D | C7 | MIR4435-2HG | LAMB1 |
| 132 | SGK1 | PRRC2C | DNAJC15 | ADCY3 |
| 133 | RAB11A | DAAM1 | SCN8A | PODN |
| 134 | PLAT | CLSTN3 | TWIST1 | ADH1B |
| 135 | RPS6KA3 | CCL19 | COG6 | PLSCR4 |
| 136 | FLNC | INO80D | PCED1B | ABTB2 |
| 137 | YWHAG | ATP8B4 | C9orf3 | ALDH1A1 |
| 138 | ITGAV | ALPK1 | MAP3K4 | PAK3 |
| 139 | MAP2K3 | NOVA1 | SMC6 | NBEA |
| 140 | IL1R1 | COL4A4 | DIO2 | ITM2B |
| 141 | ADAM17 | PITPNC1 | TTC3 | TNRC6B |
| 142 | HIF1A-AS2 | PCDH11X | ZMYM4 | TRERF1 |
| 143 | PITPNM2 | RPS11 | SAMD3 | STXBP4 |
| 144 | TNFRSF12A | APBB1IP | STARD4-AS1 | PRKCH |
| 145 | HEG1 | TNFSF10 | WWC1 | SLC8A1 |
| 146 | IQGAP1 | RPS6KL1 | UNC5B | PITPNC1 |
| 147 | VGLL3 | PDE1C | LINC01060 | CALCRL |
| 148 | TP53BP2 | GAPDH | DGKI | C1S |
| 149 | PLEKHA7 | NFKBIA | BBX | MT1X |
| 150 | AXL | CD82 | SSH1 | JADE1 |
| 151 | PHF20 | TLE2 | KCNQ1OT1 | PTPRK |
| 152 | KIF1B | SRRM2 | GXYLT2 | PDE7B |
| 153 | LPP | TMEM176B | GPR63 | ACACB |
| 154 | MCL1 | C3 | F13A1 | OGFRL1 |
| 155 | PPP1R12B | PYHIN1 | WLS | CLIP4 |
| 156 | ASAP2 | HMCN2 | PLS3 | GAB1 |
| 157 | GLS | RPS8 | SLC24A2 | PLEKHA5 |
| 158 | ATP1A1 | RPL13 | EPC1 | SMIM14 |
| 159 | CLIP1 | RPL10 | KIFAP3 | TXNIP |
| 160 | TRIO | IRF3 | COL7A1 | NCOA1 |
| 161 | LINC00968 | SMAP2 | VGLL4 | RBMS1 |
| 162 | ATP2B4 | ADGRE5 | PDZRN3 | SDCCAG8 |
| 163 | PTPN14 | PER1 | PRDM1 | STK24 |
| 164 | RYBP | PLEC | COL8A1 | FOXP2 |
| 165 | HECTD2 | ZDHHC14 | ISM1 | ABCA9-AS1 |
| 166 | ADAM9 | PHPT1 | ZNF609 | COLGALT2 |
| 167 | RAI14 | EPHB1 | CLCN3 | KDM6A |
| 168 | UCHL3 | FAM189A1 | ADAM22 | PTPN13 |
| 169 | DDX21 | RPLP0 | ACVR1 | MATN2 |
| 170 | XYLT1 | TXNRD1 | TMEM45A | CD47 |
| 171 | GREB1L | LINC00092 | PRDM6 | ARHGAP26 |
| 172 | TGFBR1 | SPATA6L | ETV6 | MBP |
| 173 | KPNA4 | EEF2 | AEBP1 | DANT2 |
| 174 | DMD | ABHD17C | TANC2 | KIF5C |
| 175 | PDZRN4 | ART4 | SRPK2 | CNKSR2 |
| 176 | IPO7 | NFKB2 | DDR2 | KCNT2 |
| 177 | SEPT9 | ARRDC3 | WIPF1 | ABCC9 |
| 178 | YBX3 | HNMT | PRKD1 | RALGAPA1 |
| 179 | RAMP1 | CLU | SPATS2 | IL6ST |
| 180 | IGF2BP2 | SPIC | RFX8 | ECHDC2 |
| 181 | LDHA | TOM1L1 | MSC-AS1 | SYNPO2 |
| 182 | ATP11A | FTL | MMP14 | TBC1D5 |
| 183 | PCDH7 | MYL6 | ZNF292 | ELMO1 |
| 184 | PRKCA-AS1 | RPS2 | IGFBP5 | GFOD1 |
| 185 | SERTAD2 | DOCK8 | ANO4 | MARCH2 |
| 186 | DENND4A | COLQ | MAML3 | FLRT2 |
| 187 | WISP1 | GAK | RAI14 | KLF12 |
| 188 | IL6R | KLHL25 | HECW2 | RBM26 |
| 189 | HIPK2 | SORCS1 | PRR5L | KCNN3 |
| 190 | HIVEP2 | PLXNB2 | MDFIC | PDE3A |
| 191 | ACLY | SLC9A9 | PCCA | NAALADL2 |
| 192 | PLAUR | AHNAK | ETV1 | INSR |
| 193 | ANO6 | DDX24 | SIPA1L3 | AUH |
| 194 | CTNNA1 | IL34 | CAMK4 | PLCL2 |
| 195 | PDLIM3 | EPAS1 | VEZT | CDON |
| 196 | CAV2 | FLNB | UHRF2 | PTPN12 |
| 197 | MSRB3 | SLIT3 | GUCY1A2 | PRKAG2 |
| 198 | MTHFD1L | EPHA2 | PHF21A | ADAMTS15 |
| 199 | COBLL1 | ETS2 | GPC6 | OAF |
| 200 | DPYSL3 | FAM20A | NBAT1 | KLHL13 |

TABLE 5
Gene Set Enrichment Analysis for malignant cNMF programs ranked by increasing FDR q-value. Threshold FDR q-value < 0.05.

| Program | Gene ontology term | Q-value |
|---|---|---|
| State | | |
| Cycling (S) | FISCHER_DREAM_TARGETS [968] | 1.20E−262 |
| | GO_CELL_CYCLE [1881] | 2.59E−131 |
| | GO_CELL_CYCLE_PROCESS [1422] | 4.00E−114 |
| | REACTOME_CELL_CYCLE [693] | 2.67E−104 |
| | GO_MITOTIC_CELL_CYCLE [1053] | 6.56E−103 |
| | GO_DNA_METABOLIC_PROCESS [943] | 8.04E−95 |
| | GO_DNA_REPLICATION [273] | 1.11E−94 |
| | REACTOME_CELL_CYCLE_MITOTIC [561] | 1.96E−88 |
| | BENPORATH_CYCLING_GENES [648] | 2.31E−88 |
| | GO_CHROMOSOME_ORGANIZATION [1253] | 4.89E−76 |
| | GO_DNA_DEPENDENT_DNA_REPLICATION [151] | 1.83E−75 |
| | FISCHER_G1_S_CELL_CYCLE [200] | 2.19E−70 |
| | REACTOME_DNA_REPLICATION [128] | 5.56E−45 |
| | REACTOME_S_PHASE [162] | 7.71E−45 |
| Cycling (G2/M) | FISCHER_DREAM_TARGETS [968] | 1.43E−230 |
| | FISCHER_G2_M_CELL_CYCLE [236] | 2.86E−184 |
| | BENPORATH_CYCLING_GENES [648] | 8.75E−133 |
| | GO_MITOTIC_CELL_CYCLE [1053] | 4.64E−116 |
| | GO_CELL_CYCLE | 3.68E−112 |
| | GO_CELL_CYCLE_PROCESS | 2.83E−103 |
| | REACTOME_CELL_CYCLE | 1.37E−97 |
| | GO_CELL_DIVISION | 3.44E−96 |
| | HALLMARK_G2M_CHECKPOINT | 8.93E−95 |
| | WHITFIELD_CELL_CYCLE_G2 | 1.78E−87 |
| | WHITFIELD_CELL_CYCLE_G2_M | 1.01E−82 |
| | REACTOME_M_PHASE | 8.46E−78 |
| | GO_NUCLEAR_CHROMOSOME_SEGREGATION | 1.18E−66 |
| | GO_MITOTIC_SISTER_CHROMATID_SEGREGATION | 6.39E−66 |
| | GO_SISTER_CHROMATID_SEGREGATION | 2.70E−65 |
| MYC signaling | GO_RNA_BINDING | 1.29E−49 |
| | WEI_MYCN_TARGETS_WITH_E_BOX | 7.89E−43 |
| | GO_NCRNA_METABOLIC_PROCESS | 5.71E−41 |
| | GO_RIBONUCLEOPROTEIN_COMPLEX_BIOGENESIS | 1.02E−38 |
| | GO_RRNA_METABOLIC_PROCESS | 2.83E−28 |
| | REACTOME_METABOLISM_OF_RNA | 9.50E−28 |
| | BILD_MYC_ONCOGENIC_SIGNATURE | 1.36E−25 |
| | GO_RNA_PROCESSING | 1.00E−24 |
| | SCHUHMACHER_MYC_TARGETS_UP | 7.76E−24 |
| | HALLMARK_MYC_TARGETS_V1 | 4.66E−23 |
| | CACGTG_MYC_Q2 | 1.07E−19 |
| | HALLMARK_MYC_TARGETS_V2 | 3.23E−19 |
| | GO_RIBONUCLEOTIDE_BINDING | 7.33E−19 |
| | KIM_MYC_AMPLIFICATION_TARGETS_UP | 8.04E−19 |
| | DANG_BOUND_BY_MYC | 3.27E−15 |
| Adhesive | KOINUMA_TARGETS_OF_SMAD2_OR_SMAD3 | 1.47E−27 |
| | ZWANG_CLASS_3_TRANSIENTLY_INDUCED_BY_EGF | 3.41E−22 |
| | ZWANG_CLASS_1_TRANSIENTLY_INDUCED_BY_EGF | 4.29E−21 |
| | NAGASHIMA_NRG1_SIGNALING_UP | 4.29E−21 |
| | ONDER_CDH1_TARGETS_2_DN | 2.02E−18 |
| | GOMF_CADHERIN_BINDING | 2.92E−16 |
| | GOMF_CELL_ADHESION_MOLECULE_BINDING | 5.93E−16 |
| | GOCC_ANCHORING_JUNCTION | 2.92E−15 |
| | GOBP_BIOLOGICAL_ADHESION | 1.24E−14 |
| | HALLMARK_TNFA_SIGNALING_VIA_NFKB | 3.09E−13 |
| | GOCC_CELL_SUBSTRATE_JUNCTION | 3.89E−12 |
| | GOBP_REGULATION_OF_CELLULAR_COMPONENT_MOVEMENT | 4.92E−12 |
| | GOBP_LOCOMOTION | 1.81E−11 |
| | GOBP_CELL_MIGRATION | 9.41E−11 |
| | GOBP_ACTIN_FILAMENT_BASED_PROCESS | 1.49E−10 |
| Ribosomal | REACTOME_EUKARYOTIC_TRANSLATION_ELONGATION | 1.44E−162 |
| | KEGG_RIBOSOME | 6.32E−152 |
| | WP_CYTOPLASMIC_RIBOSOMAL_PROTEINS | 1.37E−150 |
| | REACTOME_EUKARYOTIC_TRANSLATION_INITIATION | 2.70E−146 |
| | GOBP_COTRANSLATIONAL_PROTEIN_TARGETING_TO_MEMBRANE | 3.07E−141 |
| | GOBP_ESTABLISHMENT_OF_PROTEIN_LOCALIZATION_TO_ENDOPLASMIC_RETICULUM | 1.28E−140 |
| | REACTOME_TRANSLATION | 5.06E−127 |
| | REACTOME_CELLULAR_RESPONSE_TO_STARVATION | 1.95E−126 |
| | REACTOME_REGULATION_OF_EXPRESSION_OF_SLITS_AND_ROBOS | 1.06E−122 |
| | GOBP_TRANSLATIONAL_INITIATION | 1.12E−122 |
| | GOBP_PROTEIN_TARGETING_TO_MEMBRANE | 6.30E−122 |
| | REACTOME_RRNA_PROCESSING | 5.03E−118 |
| | REACTOME_SIGNALING_BY_ROBO_RECEPTORS | 1.81E−113 |
| | REACTOME_CELLULAR_RESPONSES_TO_EXTERNAL_STIMULI | 4.58E−97 |
| | REACTOME_METABOLISM_OF_AMINO_ACIDS_AND_DERIVATIVES | 7.20E−96 |
| Interferon signaling | HECKER_IFNB1_TARGETS [95] | 1.72E−16 |
| | HALLMARK_INTERFERON_GAMMA_RESPONSE [200] | 1.67E−15 |
| | MOSERLE_IFNA_RESPONSE [31] | 5.54E−13 |
| | HALLMARK_INTERFERON_ALPHA_RESPONSE [97] | 3.67E−12 |
| | BOSCO_INTERFERON_INDUCED_ANTIVIRAL_MODULE [78] | 7.48E−12 |
| | GO_INNATE_IMMUNE_RESPONSE [986] | 2.00E−11 |
| | BROWNE_INTERFERON_RESPONSIVE_GENES [67] | 4.01E−11 |
| | GO_RESPONSE_TO_CYTOKINE [1225] | 9.87E−11 |
| | SANA_RESPONSE_TO_IFNG_UP [76] | 1.44E−10 |
| | GO_DEFENSE_RESPONSE [1814] | 1.37E−08 |
| | GO_RESPONSE_TO_INTERFERON_GAMMA [201] | 1.77E−08 |
| | EINAV_INTERFERON_SIGNATURE_IN_CANCER [27] | 2.89E−08 |
| | GO_RESPONSE_TO_TYPE_I_INTERFERON [99] | 3.86E−08 |
| TNF-NFkB signaling | ZHANG_RESPONSE_TO_IKK_INHIBITOR_AND_TNF_UP | 3.38E−36 |
| | CHARAFE_BREAST_CANCER_LUMINAL_VS_BASAL_DN | 8.99E−34 |
| | PHONG_TNF_RESPONSE_NOT_VIA_P38 | 3.36E−30 |
| | CHARAFE_BREAST_CANCER_LUMINAL_VS_MESENCHYMAL_DN | 1.16E−29 |
| | HALLMARK_TNFA_SIGNALING_VIA_NFKB | 3.30E−20 |
| | HINATA_NFKB_TARGETS_KERATINOCYTE_UP | 5.66E−20 |
| | GO_EPITHELIUM_DEVELOPMENT | 8.26E−20 |
| | HINATA_NFKB_TARGETS_FIBROBLAST_UP | 1.42E−18 |
| | ZHOU_INFLAMMATORY_RESPONSE_LIVE_UP | 2.30E−18 |
| | SMID_BREAST_CANCER_BASAL_UP | 3.23E−18 |
| | PHONG_TNF_RESPONSE_VIA_P38_PARTIAL | 8.49E−18 |
| | PHONG_TNF_TARGETS_UP | 5.49E−17 |
| | GO_INFLAMMATORY_RESPONSE | 3.83E−15 |
| | HALLMARK_INFLAMMATORY_RESPONSE | 4.85E−15 |
| Lineage | | |
| Acinar-like | REACTOME_SELENOAMINO_ACID_METABOLISM | 8.77E−98 |
| | MURARO_PANCREAS_ACINAR_CELL | 2.52E−97 |
| | GO_PEPTIDE_METABOLIC_PROCESS | 1.30E−53 |
| | GO_CELLULAR_MACROMOLECULE_CATABOLIC_PROCESS | 3.46E−47 |
| | GO_MACROMOLECULE_CATABOLIC_PROCESS | 1.01E−44 |
| | GNF2_SPINK1 | 2.14E−44 |
| Classical | SMID_BREAST_CANCER_BASAL_DN | 1.17E−19 |
| | GO_LIPID_METABOLIC_PROCESS | 2.42E−11 |
| | GO_PLASMA_MEMBRANE_REGION | 3.42E−10 |
| | FEVR_CTNNB1_TARGETS_UP (upon CTNNB1 deletion) | 1.81E−09 |
| | GO_DIGESTION | 1.12E−08 |
| | GO_HORMONE_METABOLIC_PROCESS | 9.98E−08 |
| | GO_BRUSH_BORDER | 1.40E−07 |
| | GO_CLUSTER_OF_ACTIN_BASED_CELL_PROJECTIONS | 4.16E−07 |
| | GO_ACTIN_FILAMENT_BASED_PROCESS | 5.70E−07 |
| | GO_STEROID_METABOLIC_PROCESS | 1.03E−06 |
| | GO_DIGESTIVE_SYSTEM_PROCESS | 1.20E−06 |
| | GO_CELL_MOTILITY | 1.23E−06 |
| | YOSHIMURA_MAPK8_TARGETS_UP | 1.29E−06 |
| | REACTOME_METABOLISM_OF_LIPIDS | 2.61E−06 |
| Basaloid | PECE_MAMMARY_STEM_CELL_UP | 2.87E−33 |
| | KEGG_RIBOSOME | 5.55E−21 |
| | WP_CYTOPLASMIC_RIBOSOMAL_PROTEINS | 8.04E−21 |
| | REACTOME_RRNA_PROCESSING | 2.95E−17 |
| | WANG_TUMOR_INVASIVENESS_UP | 1.69E−15 |
| | REACTOME_NERVOUS_SYSTEM_DEVELOPMENT | 1.08E−14 |
| | REACTOME_SIGNALING_BY_ROBO_RECEPTORS | 2.60E−14 |
| | WU_CELL_MIGRATION | 4.83E−13 |
| | HALLMARK_EPITHELIAL_MESENCHYMAL_TRANSITION | 1.91E−12 |
| | GO_CELL_SUBSTRATE_JUNCTION | 1.50E−11 |
| | GO_ANCHORING_JUNCTION | 3.12E−11 |
| Squamoid | ONDER_CDH1_TARGETS_2_DN (after CDH1 knockdown) | 8.52E−14 |
| | BOSCO_EPITHELIAL_DIFFERENTIATION_MODULE | 2.61E−09 |
| | GO_CELL_ADHESION_MOLECULE_BINDING | 3.75E−08 |
| | MODULE_298 - Keratins | 3.75E−08 |
| | GO_SKIN_DEVELOPMENT | 2.95E−07 |
| | GO_EPIDERMIS_DEVELOPMENT | 1.11E−06 |
| | SARRIO_EPITHELIAL_MESENCHYMAL_TRANSITION_DN | 1.33E−06 |
| | GO_KERATINOCYTE_DIFFERENTIATION | 1.90E−06 |
| | GO_CORNIFICATION | 2.06E−06 |
| | HP_REGIONAL_ABNORMALITY_OF_SKIN | 3.23E−06 |
| | REACTOME_FORMATION_OF_THE_CORNIFIED_ENVELOPE | 5.72E−06 |
| | GO_EPITHELIAL_CELL_DIFFERENTIATION | 6.05E−06 |
| | HP_HYPERKERATOSIS | 9.83E−06 |
| | GO_EPIDERMAL_CELL_DIFFERENTIATION | 1.11E−05 |
| | GO_INTEGRIN_BINDING | 1.23E−05 |
| | GO_EPITHELIUM_DEVELOPMENT | 1.32E−05 |
| Mesenchymal | MURARO_PANCREAS_MESENCHYMAL_STROMAL_CELL | 1.16E−114 |
| | AIZARANI_LIVER_C21_STELLATE_CELLS_1 | 3.37E−78 |
| | HALLMARK_EPITHELIAL_MESENCHYMAL_TRANSITION | 1.95E−77 |
| | HU_FETAL_RETINA_FIBROBLAST | 9.87E−76 |
| | HAY_BONE_MARROW_STROMAL | 2.43E−75 |
| | GO_EXTRACELLULAR_MATRIX | 4.54E−74 |
| | GO_COLLAGEN_CONTAINING_EXTRACELLULAR_MATRIX | 1.99E−69 |
| | NABA_MATRISOME | 3.33E−64 |
| | CUI_DEVELOPING_HEART_C3_FIBROBLAST_LIKE_CELL | 4.50E−64 |
| | GO_EXTRACELLULAR_STRUCTURE_ORGANIZATION | 2.81E−63 |
| | NABA_CORE_MATRISOME | 6.93E−57 |
| | BOQUEST_STEM_CELL_UP | 1.72E−54 |
| | LIM_MAMMARY_STEM_CELL_UP | 2.31E−46 |
| | NABA_ECM_GLYCOPROTEINS | 2.32E−31 |
| | REACTOME_COLLAGEN_DEGRADATION | 1.67E−24 |
| Neuroendocrine-like | GAO_LARGE_INTESTINE_ADULT_CA_ENTEROENDOCRINE_CELLS | 2.14E−43 |
| | MURARO_PANCREAS_ALPHA_CELL | 8.17E−38 |
| | MURARO_PANCREAS_BETA_CELL | 4.57E−24 |
| | GO_SYNAPSE | 9.63E−24 |
| | GO_NEURON_PROJECTION | 1.08E−22 |
| | REACTOME_NEURONAL_SYSTEM | 1.96E−21 |
| | MURARO_PANCREAS_DELTA_CELL | 1.67E−20 |
| | GO_SYNAPTIC_SIGNALING | 2.32E−18 |
| | GO_SOMATODENDRITIC_COMPARTMENT | 8.83E−17 |
| | GO_VOLTAGE_GATED_ION_CHANNEL_ACTIVITY | 4.22E−16 |
| | GO_VOLTAGE_GATED_CATION_CHANNEL_ACTIVITY | 5.87E−16 |
| | GO_REGULATION_OF_TRANS_SYNAPTIC_SIGNALING | 7.62E−16 |
| | GO_PEPTIDE_HORMONE_SECRETION | 2.83E−15 |
| | GO_INSULIN_SECRETION | 6.92E−12 |
| | GO_REGULATION_OF_PEPTIDE_HORMONE_SECRETION | 6.92E−12 |
| Neural-like progenitor | GOBP_NEURON_DEVELOPMENT | 1.16E−16 |
| | GOBP_NEURON_DIFFERENTIATION | 1.24E−15 |
| | GO_NEUROGENESIS | 4.44E−14 |
| | WONG_ADULT_TISSUE_STEM_MODULE | 2.81E−12 |
| | HNF1_01 | 6.55E−12 |
| | GOBP_CELL_MORPHOGENESIS_INVOLVED_IN_NEURON_DIFFERENTIATION | 1.25E−11 |
| | GOBP_ANIMAL_ORGAN_MORPHOGENESIS | 1.95E−11 |
| | GOCC_NEURON_PROJECTION | 4.51E−11 |
| | GOBP_AXON_DEVELOPMENT | 3.31E−09 |
| | GOBP_NEURON_PROJECTION_GUIDANCE | 3.45E−09 |
| | GOBP_CENTRAL_NERVOUS_SYSTEM_DEVELOPMENT | 5.58E−09 |
| | TGTTTGY_HNF3_Q6 | 6.61E−09 |
| | HNF1_C | 8.48E−09 |
| | HNF1_Q6 | 9.92E−08 |

TABLE 6
Gene Set Enrichment Analysis for fibroblast cNMF programs ranked
by increasing FDR q-value. Threshold FDR q-value < 0.05.

| Program | Gene ontology term | Q-value |
|---|---|---|
| Adhesive | KOINUMA_TARGETS_OF_SMAD2_OR_SMAD3 | 3.99E−28 |
| | GO_ANCHORING_JUNCTION | 2.61E−26 |
| | GO_CELL_SUBSTRATE_JUNCTION | 1.44E−23 |
| | GO_CELL_ADHESION_MOLECULE_BINDING | 2.21E−20 |
| | GO_CELL_MOTILITY | 1.14E−18 |
| | GO_CADHERIN_BINDING | 7.19E−18 |
| | PLASARI_TGFB1_TARGETS_10HR_UP | 1.09E−17 |
| | GO_REGULATION_OF_CELLULAR_COMPONENT_MOVEMENT | 6.57E−17 |
| | SCHUETZ_BREAST_CANCER_DUCTAL_INVASIVE_UP | 2.53E−16 |
| | GO_CELL_PROJECTION_ORGANIZATION | 7.56E−15 |
| | GO_ACTIN_CYTOSKELETON | 3.62E−14 |
| | GO_POSITIVE_REGULATION_OF_CELLULAR_COMPONENT_ORGANIZATION | 1.07E−13 |
| | GO_BIOLOGICAL_ADHESION | 8.29E−13 |
| Immunomodulatory | HALLMARK_TNFA_SIGNALING_VIA_NFKB | 7.24E−18 |
| | PHONG_TNF_TARGETS_UP | 9.70E−18 |
| | GO_RESPONSE_TO_CYTOKINE | 1.45E−14 |
| | GO_CELL_ACTIVATION | 1.00E−13 |
| | GO_DEFENSE_RESPONSE | 3.84E−13 |
| | REACTOME_INNATE_IMMUNE_SYSTEM | 1.17E−12 |
| | GO_RESPONSE_TO_ENDOGENOUS_STIMULUS | 2.56E−12 |
| | GO_INFLAMMATORY_RESPONSE | 4.17E−12 |
| | GO_SECRETION | 5.10E−12 |
| | REACTOME_CYTOKINE_SIGNALING_IN_IMMUNE_SYSTEM | 5.15E−12 |
| | GO_IMMUNE_SYSTEM_DEVELOPMENT | 7.74E−10 |
| | GO_CYTOKINE_PRODUCTION | 1.06E−09 |
| | GO_CYTOKINE_MEDIATED_SIGNALING_PATHWAY | 1.13E−09 |
| Myofibroblastic | LIM_MAMMARY_STEM_CELL_UP | 1.96E−18 |
| progenitor | GOBP_EMBRYO_DEVELOPMENT | 8.71E−16 |
| | GOBP_CIRCULATORY_SYSTEM_DEVELOPMENT | 9.55E−16 |
| | NABA_MATRISOME | 1.47E−14 |
| | GO_SKELETAL_SYSTEM_DEVELOPMENT | 3.86E−14 |
| | GO_VASCULATURE_DEVELOPMENT | 4.00E−14 |
| | REACTOME_EXTRACELLULAR_MATRIX_ORGANIZATION | 8.08E−13 |
| | GOBP_EMBRYONIC_MORPHOGENESIS | 1.05E−12 |
| Neurotropic | GO_NEUROGENESIS | 8.47E−13 |
| | GO_NEURON_DIFFERENTIATION | 1.22E−11 |
| | GO_NEURON_DEVELOPMENT | 5.01E−11 |
| | GO_NEURON_PROJECTION | 1.39E−10 |
| | GO_CELL_MORPHOGENESIS | 1.76E−10 |
| | REACTOME_NERVOUS_SYSTEM_DEVELOPMENT | 6.40E−10 |
| | GO_CELL_PROJECTION_ORGANIZATION | 1.13E−09 |
| | GO_CELL_MORPHOGENESIS_INVOLVED_IN_NEURON_DIFFERENTIATION | 1.25E−09 |
| | GO_SYNAPSE | 2.43E−09 |

### Refined Malignant Cell Classification Identifies a Novel Neural-Like Progenitor Program

In addition to the classical-like program that strongly overlapped with previously defined classical signatures^{9,69} [Moffitt classical subtype (p=8.12×10⁻¹¹; hypergeometric test), classical A subtype (p=5.61×10⁻⁶⁶), classical B subtype (p=1.00×10⁻¹⁰)], three other programs corresponded to squamoid, basaloid, and mesenchymal states separately, in lieu of a joint basal-like/squamous/quasi-mesenchymal program⁸⁻¹⁰ from bulk expression profiles. Applicant further identified in bona fide high CNA malignant cells (FIG. 2A; Tables 4-5) an acinar-like program and a neuroendocrine-like program, reminiscent of neuroendocrine-like differentiation states in other tumor types⁷⁴⁻⁷⁶. Prior bulk studies often ascribed endocrine-like or exocrine-like profiles to non-malignant cells in these less pure tumor subtypes^{6,8,10,11}, but the data show that these are present in malignant cells.

The basaloid, squamoid and mesenchymal programs were each enriched in relevant genes (FIG. 2A, Tables 4-5): epidermis development/proliferation, keratinocyte differentiation, and cornification (e.g., KRT13, KRT16, SCEL) 77-79 in the squamoid program; stemness, ribosomal proteins, rRNA processing, neurogenesis, cell migration, tumor invasiveness, cell-cell and cell-extracellular matrix (ECM) junctions, epithelial-mesenchymal transition (EMT), and metallothioneins in the basaloid program^{80,81}; and EMT, matrisome, ECM production, and stemness in the mesenchymal program^{82,83}. The squamoid and basaloid programs overlapped significantly with the Moffitt basal-like signature (p=3.72×10⁻⁹ and 1.93×10⁻³, respectively; hypergeometric) 9, but the squamoid and mesenchymal programs did not exhibit significant overlap with the bulk-defined squamous¹⁰ and quasi-mesenchymal subtypes⁸, respectively.

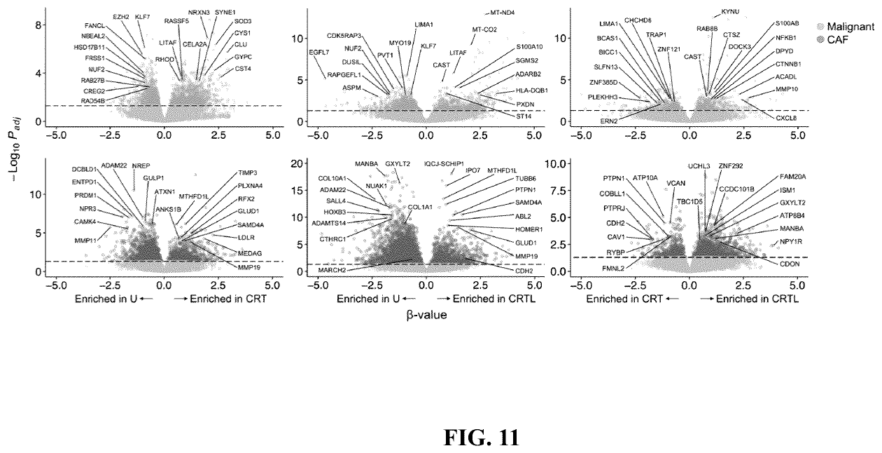

A distinct and novel neural-like progenitor program was simultaneously enriched for pathways and genes involved in neuronal development/migration/adhesion (e.g., CNTN4, CTNND2, NRXN3, RELN, SEMASA, NRCAM, AUTS2)⁸⁴⁻⁸⁹, and in tissue stem cell modules, organ morphogenesis, and hepatocyte nuclear factor activity (Table 5), known to function in embryonic and adult tissue development⁹⁰. The program was also significantly enriched in ‘brain tissue enhanced’ genes in the Human Protein Atlas (p=1.30×10⁻⁴; hypergeometric, FIG. 2C)^{91,92}. Notably, the data decouples the neural-like and neuroendocrine-like programs, which have been challenging to distinguish in studies of other malignancies^{75,93,94,95} (FIG. 2A-2B; Tables 4-5). Applicant validated the program in situ by multiplexed immunofluorescence, showing that a subset of malignant cells/glands co-express pan-cytokeratin and NRXN3, a program gene typically expressed in neuronal and glial cells of the cerebral cortex and caudate^{91,92} (FIG. 2C, 2D, FIG. 11, Methods). Thus, neural-enriched proteins can be expressed within invasive epithelia. The features of this program are consistent with the frequent and diverse somatic aberrations in genes linked to axonal guidance⁹⁶, tumor-nerve crosstalk, and the high prevalence of perineural invasion (PNI) observed in PDAC⁹⁷. For example, class 3 semaphorins, such as the program gene SEMA3E, are amplified or mutated in >20% of PDAC⁹⁶, and genes that are differentially expressed between an independent cohort of untreated PDAC tumors with (n=134) versus without (n=25) PNI⁹⁸ have significantly higher program weights (p=0.006; Kolmogorov-Smirnov test, Methods; FIG. 15).

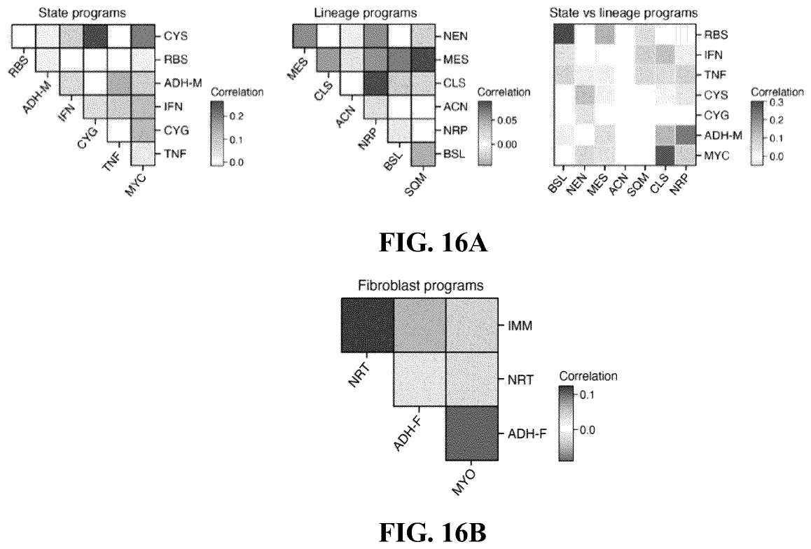

There were seven additional ‘cell state’ programs in malignant cells (FIG. 2A; Tables 4-5). These spanned cell cycle programs in S and G2/M phases; a ribosomal program; an interferon program enriched in type 1 and 2 interferon response genes and other cytokines; a TNF-NFκB signaling program; a MYC signaling program; and a cell adhesion and motility program (FIG. 2A; Tables 4-5). Inter-program correlation scores showed strong associations between the ribosomal and basaloid; MYC and classical; adhesive and neural-like progenitor; and among the cycling (S and G2/M) and MYC programs, some of which is consistent with prior work^{39,80,99,100} (FIG. 16A).

### Expanded CAF Classification Reveals Four Programs

Among the four CAF programs, the ACTA2-enriched myofibroblastic progenitor program overlapped with a published myCAF signature (p=1.04×10⁻¹²; hypergeometric)+ (FIG. 2A; Tables 4, 6) but was differentiated by an enrichment of genes involved in embryonic mesodermal development and Wnt signaling (e.g., RUNX1, RUNX2, LEF1, SALL4, WNT5A, NKD1, FOXP1)¹⁰¹⁻¹⁰⁸. The neurotropic, immunomodulatory, and adhesive programs all overlapped with the single-cell iCAF signature (p=1.90×10⁻¹⁰, 3.22×10⁻¹⁴, and 3.85×10⁻⁴, respectively; hypergeometric) but not the myCAF signature (p-0.149, 0.851, and 0.851, respectively) 49, suggesting they may reflect different iCAF subsets. None of the programs significantly overlapped with the single-cell apCAF signature⁴⁹. In addition, the CAF programs overlapped with cross-tissue fibroblast signatures⁷²: the myofibroblastic progenitor program with the LRCC15⁺ myofibroblast (p=8.71×10⁻⁴²; hypergeometric) and COL3Al⁺ myofibroblast (p=2.86×10⁻¹³) signatures; the neurotropic program with the P116⁺ adventitial (p=7.65×10⁻²⁴) and NPNT alveolar (p=1.62×10⁻³⁷) signatures; the immunomodulatory program with the CCL19⁺ colitis (p=1.30×10⁻²⁹) and ADAMDEC1⁺ colitis (p=5.28×10⁻¹²) signatures; and the cell adhesion program with the PI16⁺ adventitial (p=2.58×10⁻⁵¹) and LRRC15⁺ myofibroblast (p=1.73×10⁻¹²) signatures.

Moreover, the immunomodulatory fibroblast program was enriched in pathways involving cytokine production/response, inflammation, and TNF-α/NF-κB signaling, and included (XCL12, CCL19 and CCL21, all of which play roles in the pathogenesis of pancreatic cancer^{109,110,111,112}; IL34, which induces proliferation and differentiation in monocytes and macrophages¹¹³; and several members of the complement pathway that may affect neutrophil recruitment^{114,115} (FIG. 2A; Tables 4, 6). The cell adhesion program featured pathways involved in cell-cell/-ECM adhesion (e.g., CDH2), cytoskeletal remodeling, and motility. The neurotropic program was enriched for genes involved in neurogenesis, neuron differentiation, and neuronal projections (FIG. 2A; Tables 4, 6). CAFs have been linked directly and indirectly to neurotropic phenomena in pancreatic cancer¹¹⁶.

### Treatment-Associated Patterns of Malignant and CAF Program Expression

Neoadjuvant therapy was associated with significant differences in the expression of malignant and CAF programs at the patient level (FIG. 3A-3B; Methods). The malignant neural-like progenitor (p_{adj}-6.98×10⁻³; Mann-Whitney) and neuroendocrine-like (p_{adj}=1.39×10⁻²) programs were significantly higher in CRT vs. untreated, whereas the classical (p_{adj}=1.33×10⁻³) and squamoid (p_{adj}=3.34×10⁻³) programs were lower (FIG. 3A-3B). Neoadjuvant CRTL vs. untreated also showed higher expression of the neural-like progenitor program (p_{adj}=2.30×10⁻³; Mann-Whitney) and lower expression of the classical (p_{adj}=7.78×10⁻³), squamoid (p_{adj}=1.37×10⁻³), and basaloid (p_{adj}=1.52×10⁻²) programs (FIG. 3A-3B). Applicant validated the increased expression of the neural-like progenitor program after treatment in organoids derived from an untreated tumor (PDAC_U_12), treated with a 10-day ex vivo chemoradiotherapy regimen (Methods), and profiled by snRNA-seq pre-vs. post-treatment (p=1.33×10⁻¹⁵, Mann-Whitney; FIG. 3D).

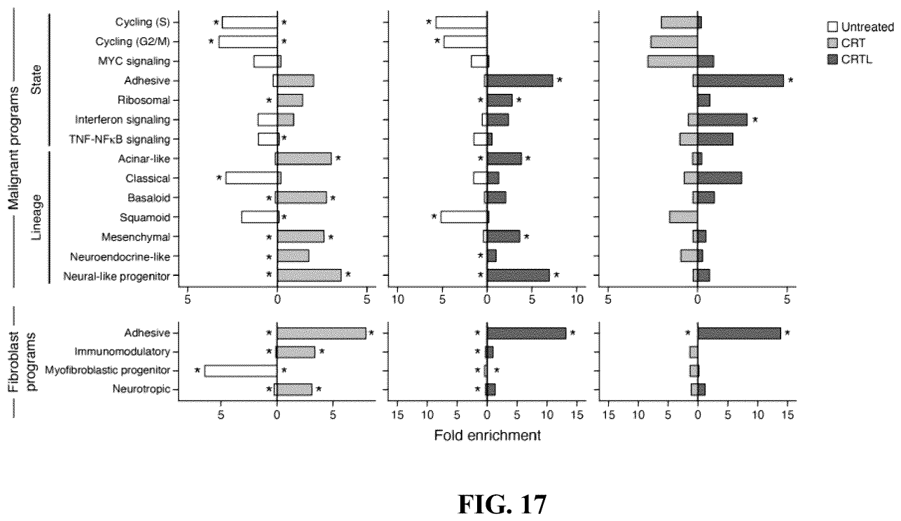

In the CAF compartment, treatment was associated with lower myofibroblastic progenitor program expression (CRT vs. untreated, p_{adj}=1.52×10⁻³; CRTL vs. untreated, p_{adj}=4.86×10⁻³; Mann-Whitney) and higher adhesive program expression (CRT vs. untreated, p_{adj}=1.93×10⁻²; CRTL vs. untreated, p_{adj}=2.97×10⁻³; CRTL vs. CRT, p_{adj}=3.50×10⁻²) (FIG. 3a-3b). The immunomodulatory CAF program was higher in CRT vs. CRTL (p_{adj}=9.47×10⁻³; Mann-Whitney). These differences were consistent with differential gene expression among untreated, CRT, and CRTL malignant cells and CAFs (FIGS. 11 and 17; Methods).

Expression of programs post-treatment was also associated with clinical response. Applicant annotated each of the 25 treated samples based on the patient's surgical pathology treatment response grade (poor, minimal, or moderate; Methods) irrespective of treatment regimen, and scored their remaining (residual) malignant cells for the seven malignant lineage programs. The residual malignant cells in patients with moderate response were enriched in the neural-like progenitor program and depleted in the classical-like and squamoid programs relative to untreated tumors (p_{adj}<0.01, Mann-Whitney) (FIG. 3C). The neural-like progenitor program score monotonically increased from untreated to poor response to minimal/moderate response, whereas the classical-like and squamoid programs monotonically decreased (FIG. 3C).

Neural-Like Progenitor Malignant Program is Associated with Poor Clinical Outcomes

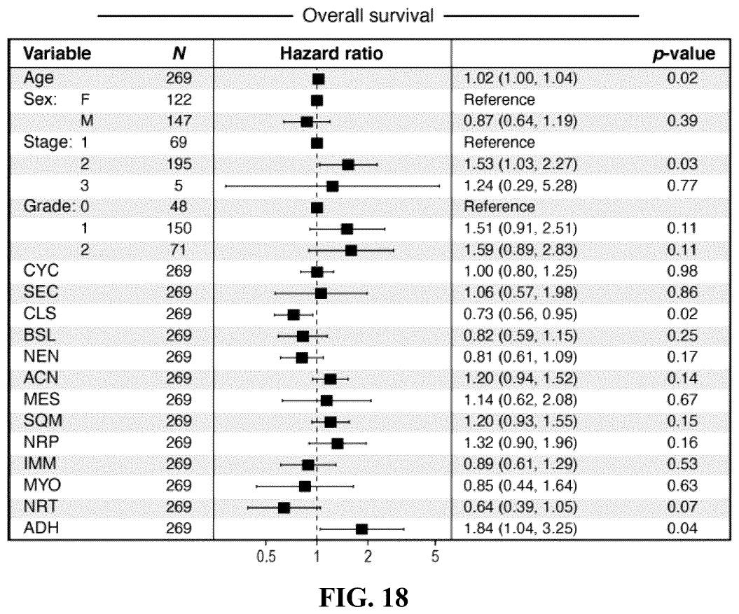

To assess the potential prognostic relevance of the malignant and CAF programs¹¹⁷, Applicant scored them in clinically-annotated bulk RNA-seq data from patients with untreated, resected primary PDAC from TCGA¹¹ and PanCuRx^{14,69} (n=266; Methods). Applicant performed a multivariable Cox regression analysis of the time to progression (TTP) and overall survival (OS) endpoints with age, sex, stage, grade, and the CAF and malignant programs as covariates. Age, sex, stage, and grade were not prognostic for TTP (FIG. 3E). The neural-like progenitor (HR 1.62, 95% CI: 1.08-2.42) and squamoid programs (HR 1.35, 95% CI: 1.02-1.78) were associated with shorter TTP, whereas the classical (HR 0.61, 95% CI: 0.46-0.82) and immunomodulatory programs (HR 0.59, 95% CI: 0.39-0.89) were associated with longer TTP (FIG. 3E). For OS, age (HR 1.02, 95% CI: 1.00-1.04) and the adhesive CAF program (HR 1.84, 95% CI: 1.04-3.25) were associated with shorter survival, while the classical program (HR 0.73, 95% CI: 0.56-0.95) was associated with longer survival. The neural-like progenitor (HR 1.32, 95% CI: 0.90-1.96) and squamoid programs (HR 1.20, 95% CI: 0.93-1.55) trended towards a negative association with OS but did not reach significance (FIG. 18). These findings parallel an association between the neuronal subtype and poor outcomes in bladder cancer⁹³. Applicant cautions, however, that applying snRNA-seq based programs to bulk profiles may be confounded by non-malignant cells (e.g., endocrine, intrapancreatic neurons) that express some of the programs, especially neuroendocrine, neural-like progenitor, and mesenchymal, at relatively high levels.

### Mapping Cell Types and Expression Programs to Tumor Architecture by Spatial Profiling

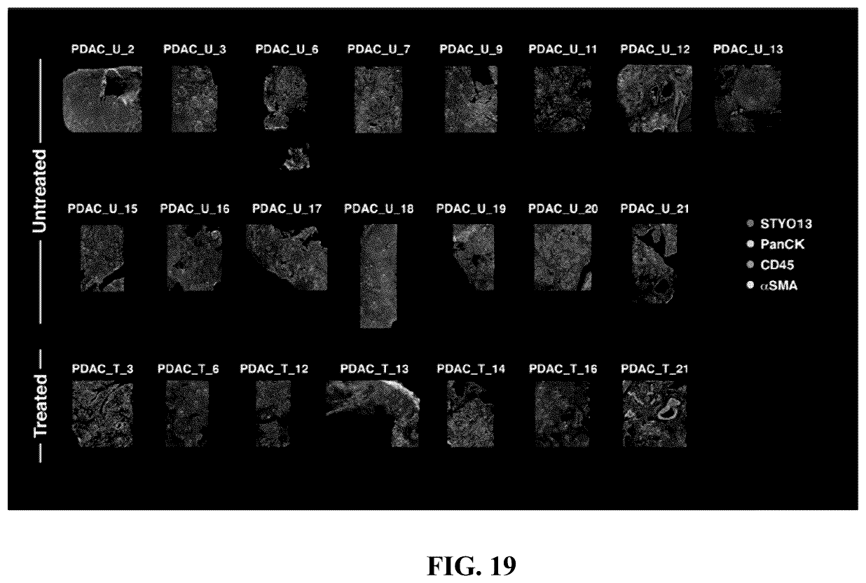

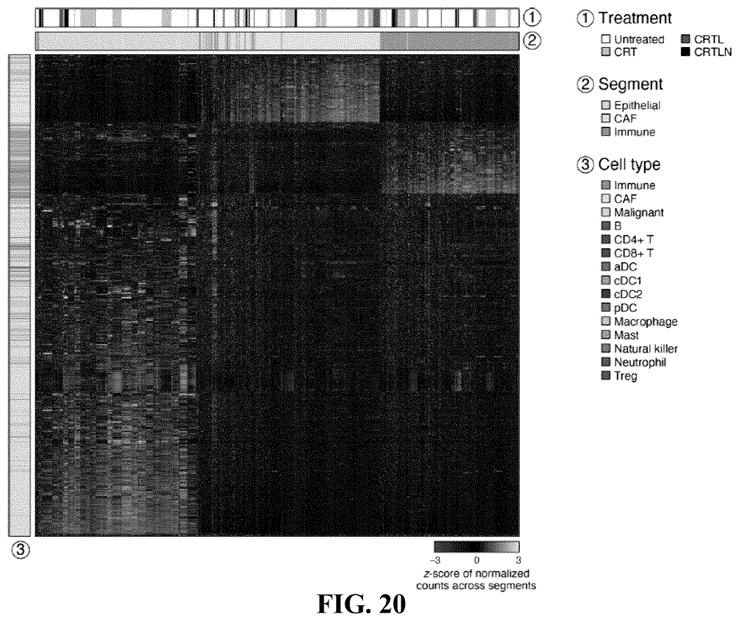

To decipher how cells and expression programs are spatially organized in multicellular communities^{11,118,119}, Applicant performed digital spatial profiling (DSP) with the NanoString GeoMx human whole transcriptome atlas (WTA; 18,269 mRNA targets). Applicant hybridized UV-photocleavable barcode-conjugated RNA ISH probes on FFPE sections to capture and profile mRNA counts from user-defined regions of interest (ROIs) (FIG. 4A; FIG. 19; Methods)⁴³. Applicant used four-color immunofluorescence to select ROIs with diverse patterns of neoplastic cells, CAFs, and immune cells (FIG. 4A; FIG. 19; Methods); created custom areas of illumination (AOI) for each cell type segment within an ROI; cleaved and collected barcodes from each AOI, and quantified barcode abundance by sequencing (Methods). Applicant analyzed 21 tumors by DSP, 18 with matching snRNA-seq (FIG. 1A, FIG. 4A; FIG. 19; Methods)⁴³, and deconvolved the data with snRNA-seq cell type signatures. The epithelial, CAF, and immune AOIs clustered appropriately by cell type (FIG. 20). Applicant then mapped the expression of each malignant and CAF program onto the ROIs (FIG. 4B), with 54% of the top 200-weighted program genes detected above background (Methods).

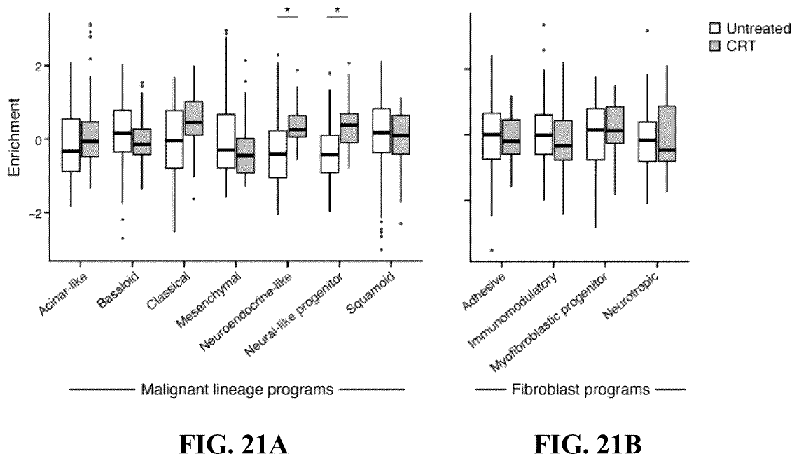

Most snRNA-seq programs were more variable between independent patient tumors (inter-patient dispersion) than across different ROIs from the same tumor (intra-patient dispersion), except for the mesenchymal, immunomodulatory, and myofibroblastic progenitor programs (FIG. 4B, right; Methods). Only the neural-like progenitor and neuroendocrine-like malignant programs were enriched in ROIs from CRT vs. untreated samples (p=1.07×10⁻² and 4.98×10⁻², respectively; linear mixed-effects model with patient ID as random effect; FIG. 21A-21B), consistent with Applicant's snRNA-seq analysis (FIG. 3B).

### Distinct Multicellular Communities of Malignant, Fibroblast and Immune Cells

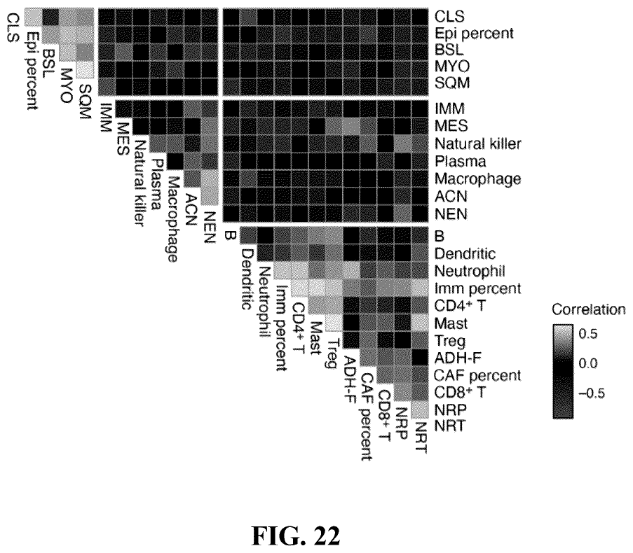

To identify spatial associations across cells of different types or programs, Applicant correlated each pair of features (program scores or inferred cell type proportions) across the ROIs to yield a spatial co-variation matrix of the malignant lineage programs (FIG. 2A), CAF programs (FIG. 2A), proportions of immune cell types (by deconvolution using snRNA-seq signatures, Methods), and the percent ROI area occupied by the malignant, fibroblast, and immune segments (FIG. 4C). Unsupervised clustering identified three multicellular communities (FIG. 4C; Methods) with distinct malignant, stromal and immune features. Community 1 (“treatment-enriched”) was characterized by an association among the neural-like progenitor and neuroendocrine-like malignant programs, the neurotropic CAF program, and CD8 T cells-which were all enriched with treatment in snRNA-seq—as well as the mesenchymal and acinar malignant programs and the immunomodulatory CAF program (FIG. 4C-4D). Community 2 (“squamoid-basaloid”) featured an association of the squamoid and basaloid malignant programs with a diverse set of lymphoid and myeloid cell types (FIG. 4C-4D), higher epithelial and immune content, and lower CAF content. Community 3 (“classical”) exhibited an association among the classical malignant program, the myofibroblastic progenitor and adhesive CAF programs, macrophages, neutrophils, and conventional type 2 dendritic cells (cDC2s) (FIG. 4C-4D), as well as higher CAF and lower immune proportions. Clustering of patient-level features in the snRNA-seq data (FIG. 22) recapitulated some of these associations.

The three communities highlight broad canonical features, but finer pair-wise associations were recovered by more granular analysis. For example, the classical malignant program was not strongly correlated with macrophage and neutrophil prevalence despite being in the same community (FIG. 4C). Applicant therefore computed the fold-change of each deconvolved immune cell type proportion between the highest quartile-scoring and the lowest quartile-scoring ROIs for each malignant and CAF program (FIG. 5A; Methods). Consistent with community 1 “treatment-enriched” (FIG. 4C-4D), ROIs with high neural-like progenitor and/or neuroendocrine-like program scores were significantly enriched with CD8⁺ T cells and depleted of conventional type 1 dendritic cells (cDC1s); the former was also enriched in cDC2s (FIG. 5A). In contrast, high-scoring squamoid, basaloid, or mesenchymal ROIs were depleted of CD8⁺ T cells; the squamoid program associated with B cells and the basaloid and mesenchymal programs associated with all DC subsets except cDC2s (FIG. 5A). ROIs with high classical program scores were enriched with CD4⁺ T cells (FIG. 5A). Similarly, the neurotropic CAF program was positively associated with CD8⁺, CD4⁺, and regulatory T cells and negatively with activated DCs and cDCIs; the myofibroblastic progenitor and adhesive programs were only positively associated with macrophages and cDC2s, respectively; and the immunomodulatory program was positively associated with activated DCs, cDC1s, plasmacytoid DCs, and plasma cells and negatively with CD4⁺ T cells and macrophages (FIG. 5A). Thus, spatial associations involved both broad multicellular communities, reflected in the clustering analysis (FIG. 4C-4D), and finer features related to pairs of specific cell types and programs (FIG. 5A).

Finally, to uncover interactions among the malignant, CAF, and immune compartments that may facilitate therapeutic resistance, Applicant identified spatially-defined receptor-ligand (RL) pairs co-expressed across ROIs in either CRT or untreated samples (FIG. 5B; FIG. 23; Table 3; Methods). Although some RL pairs were well-correlated in both untreated and CRT specimens, many pairs were differentially correlated by treatment status (Table 3).

### DISCUSSION

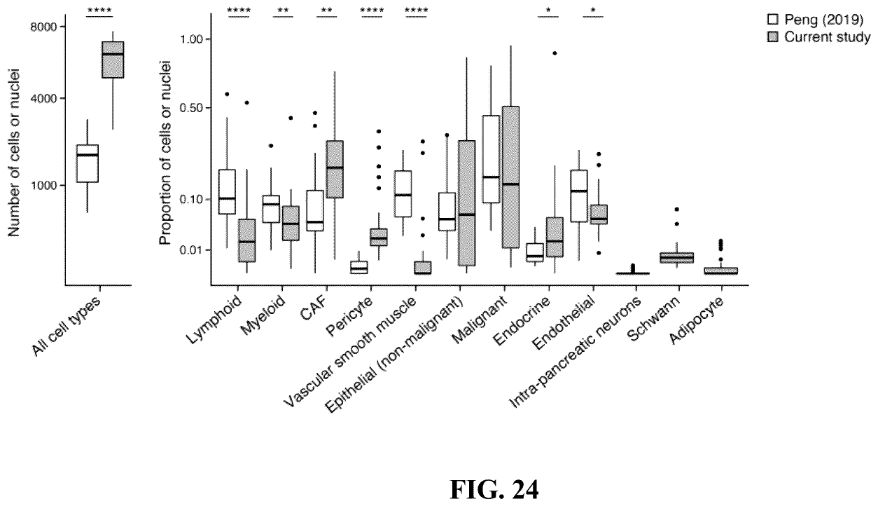

In this study, Applicant used snRNA-seq and digital spatial profiling of a large cohort of primary PDAC to construct a detailed classification of tumor composition, malignant and CAF programs, immune milieu, and clinical outcomes (FIG. 5C). Applicant's snRNA-seq approach was compatible with untreated and heavily pre-treated frozen specimens (FIG. 1C-1D; FIG. 6A-6B; Table 2) and may yield better in situ cell type representation than scRNA-seq (FIG. 24)⁵¹, albeit with some differential immune subset capture (FIG. 1E; FIG. 8). Analysis of some immune subsets may benefit from further application-specific optimization and complementary in situ approaches.

Evidence of putative intermediate states (ADM, atypical ductal) supports a path of transformation from acinar to ADM to ductal to atypical ductal to malignant cells in patient tumors (FIG. 1D, 1G, 1H). The co-existence of precursor and malignant states in the same tumors may be secondary to field effect; elucidating this would require studies focused on precursor lesions and non-malignant tissue adjacent to tumors.

Applicant's de novo expression programs provide a refined and expanded cell taxonomy of malignant cells and CAFs in PDAC (FIG. 2A). In addition to previously reported subtypes such as myCAFs^{49,71} and malignant classical⁸⁻¹⁰, Applicant's analysis partitioned an aggregate “basal-like” /“squamous” /“quasi-mesenchymal” subtype⁸⁻¹⁰ into distinct squamoid, basaloid, and mesenchymal programs; revealed neuroendocrine-like and acinar-like programs that support the existence of the aberrantly-differentiated endocrine exocrine (ADEX) subtype^{6,10}, and uncovered a novel neural-like progenitor program, which Applicant validated in situ (FIG. 2C-2D).

While Applicant expanded on prior studies by dissecting cellular ecosystems in both untreated and treated tumors, Applicant was limited by the lack of matched pre- and post-treatment specimens, treatment heterogeneity, sample size for certain treatment groups, and technical challenges associated with single nucleus extractions. Nevertheless, with a large cohort and statistical adjustments to account for patient origin of each cell, the neural-like progenitor program was enriched in all post-treatment groups, including organoids treated ex vivo (FIG. 3B-3D), and was associated with the shortest TTP (and trended towards worse OS) in bulk profiles from two independent cohorts (FIG. 3E; FIG. 8).

The mechanisms through which neural-like progenitor cells may resist treatment remain open-ended, including whether they are cancer-intrinsic, derived from TME interactions, or both. Several program genes are involved in drug efflux, negative regulation of cell death, and chemoresistance (e.g., ABCB1, BCL2, PDGED, SPP1)¹²⁰⁻¹²⁶. Moreover, neuronal migration and axonal guidance genes (e.g., SEMA3E, RELN, SEMA5A)^{86,127,128} and PNI-associated genes may reflect tumor-nerve crosstalk (FIG. 15), which has been associated with dissemination, post-treatment recurrence, and metabolic support^{97,129,130}. The NFIB transcription factor is a member of the program and promotes pro-metastatic neuronal gene expression programs in other cancer types¹³¹. Combining snRNA-seq with spatial whole transcriptome profiling identified how the different malignant and stromal programs relate to each other and to immune cell composition (FIGS. 4 and 5). The colocalization of neural-like progenitor and neuroendocrine-like malignant programs with the neurotropic CAF program and CD8⁺ T cells in one multicellular community suggest a functional interplay among these cell types/states.

Spatially-defined receptor-ligand interactions, especially those that are differentially correlated between untreated and CRT tumors (FIG. 5B; Table 3) may functionally underpin these communities. The CXCL12-CXCR4 interaction^{133,134} is the most differentially correlated RL pair between epithelial and immune cells (FIG. 5B; Table 3), and supports investigation of AMD3100, a small-molecule CXCR4 inhibitor¹³³⁻¹³⁵, as a potential adjunct to neoadjuvant CRT. Within epithelial ROIs in CRT tumors, correlated RL pairs involved ERBB2 (e.g., HRG-ERBB2, HBEGF-ERBB2, LICAM-ERBB2, NRG1-ERBB2, FIG. 23; Table 3), consistent with the role of HER2 signaling in resistance to chemotherapy in PDAC cell lines¹³⁶⁻¹⁴⁰, suggesting that HER2 inhibition may have therapeutic efficacy in concert with neoadjuvant CRT (FIG. 23; Table 3), despite previous challenges in clinical trials^{141-143,144} .

TABLE 3
Select spatially defined receptor ligand pairs as a function of treatment. Cell-type specific Spearman's
rank correlation coefficients of expression (ρ) are provided based on treatment group.

| Receptor- | Receptor | Ligand | ρ | ρ | | |
|---|---|---|---|---|---|---|
| Ligand Pair | Compartment | Compartment | untreated | CRT | Δρ | Description |
| CXCR4- | Immune | Epithelial | −0.19 | 0.41 | 0.60 | CXCL12 expression on tumor cells has |
| CXCL12 | | | | | | been shown to suppress the migration of |
| | | | | | | immune cells, such as cytotoxic |
| | | | | | | lymphocytes, and clinical inhibition of |
| | | | | | | CXCR4 with AMD3100 induced an |
| | | | | | | integrated immune response¹³³ |
| TNFRSF25- | Immune | Epithelial | −0.17 | 0.31 | 0.48 | TNFRSF25 agonism has costimulatory |
| TNFSF15 | | | | | | effects on CD8⁺ T-cells, promoting |
| | | | | | | accumulation, expansion, and cytotoxic |
| | | | | | | effector function, consistent with the |
| | | | | | | enrichment of CD8⁺ T lymphocytes in |
| | | | | | | the “treatment-enriched” community¹⁷¹ |
| CXCR2-CXCL5 | Immune | Epithelial | 0.16 | 0.38 | 0.22 | Impacts neutrophil recruitment at |
| CXCR2-CXCL3 | Immune | Epithelial | 0.10 | 0.43 | 0.33 | inflammatory sites¹⁷² |
| ILIR1-IL1A | Fibroblast | Epithelial | −0.080 | 0.45 | 0.53 | Upregulation after treatment is consistent |
| | | | | | | with the pro-inflammatory effects of |
| | | | | | | cytotoxic therapy¹⁷³, though prior studies |
| | | | | | | have also associated IL-1α signaling with |
| | | | | | | a pro-tumorigenic niche¹⁷⁴ |
| TNFRSF21-TNF | Fibroblast | Epithelial | 0.0042 | 0.44 | 0.44 | TNF/NF-κB signaling has been shown to |
| | | | | | | be upregulated in inflammatory CAFs⁴⁹ |
| GFRA1-GDNF | Fibroblast | Epithelial | 0.019 | 0.42 | 0.40 | GDNF and soluble GFRα1 (albeit from |
| | | | | | | nerves) have been shown to cooperate to |
| | | | | | | activate RET on cancer cell surface, |
| | | | | | | which enhances migration and perineural |
| | | | | | | invasion¹⁷⁵ |
| DPP4-NPY | Epithelial | Epithelial | 0.44 | −0.34 | −0.78 | NPY is cleaved by the peptidase DPP4 |
| | | | | | | followed by preferential signaling |
| | | | | | | through the NPY2R receptor¹⁷⁶, which is |
| | | | | | | upregulated in PanINs and invasive |
| | | | | | | adenocarcinoma relative to normal |
| | | | | | | pancreas in mice and humans¹⁷⁷ |
| ERBB2-HRG | Epithelial | Epithelial | −0.61 | 0.20 | 0.81 | Although HER2 is not generally |
| ERBB2-HBEGF | Epithelial | Epithelial | −0.46 | 0.35 | 0.81 | associated with direct ligands, |
| ERBB2-L1CAM | Epithelial | Epithelial | −0.52 | 0.19 | 0.71 | interactions with HRG have been shown |
| ERBB2-NRG1 | Epithelial | Epithelial | −0.60 | 0.036 | 0.64 | to modulate its signaling activity¹³⁷⁻¹⁴⁰ |
| IGF1R-IGF2 | Epithelial | Fibroblast | −0.18 | 0.31 | 0.49 | IGF2 from myofibroblasts has been |
| | | | | | | shown to boost IGF1R-driven signaling |
| | | | | | | in cholangiocarcinoma. IGF2-IGF1R |
| | | | | | | signaling associates with cancer stem |
| | | | | | | cell-like phenotypes and resistance to |
| | | | | | | EGFR inhibition¹⁷⁸ |
| TRAF2-TNF | Epithelial | Fibroblast | 0.12 | 0.46 | 0.34 | In Ras-transformed cancers, TRAF2 may |
| | | | | | | promote resistance to stress-induced |
| | | | | | | apoptosis and associates with enhanced |
| | | | | | | NF-κB signaling¹⁷⁹ |
| CXCR2-CXCL2 | Epithelial | Fibroblast | 0.38 | 0.039 | −0.34 | Heterozygous knockout of Cxcr2 |
| | | | | | | significantly extended the survival of |
| | | | | | | PDAC mice¹⁸⁰. In addition, genetic |
| | | | | | | ablation and chemical inhibition of |
| | | | | | | CXCR2 in the KPC mouse model of |
| | | | | | | pancreatic cancer significantly reduces |
| | | | | | | metastatic potential, enhances infiltration |
| | | | | | | of T cells, and extends survival in |
| | | | | | | combination with anti-PD1 therapy¹⁸¹. |
| CXCR2-CXCL8 | Epithelial | Fibroblast | 0.36 | 0.14 | −0.22 | CXCR2-CXCL8 signaling can promote |
| | | | | | | angiogenesis, metastasis, and activate |
| | | | | | | numerous intracellular signaling |
| | | | | | | pathways¹⁸² |
| CXCR1-CXCL6 | Immune | Fibroblast | −0.20 | 0.37 | 0.57 | CXCR1 agonism on neutrophils can |
| | | | | | | promote neutrophil extracellular traps |
| | | | | | | that protect tumor cells from immune- |
| | | | | | | mediated cytotoxicity¹⁸³ |
| CCR7-CCL19 | Immune | Fibroblast | −0.061 | 0.41 | 0.47 | CCL19 can bind to CCR7 on CD4⁺ T |
| | | | | | | cells and promote survival of naïve T |
| | | | | | | cells as they enter the lymph node and |
| | | | | | | interact with antigen-presenting cells¹⁸⁴. |
| | | | | | | CCL19-producing fibroblasts have been |
| | | | | | | shown to restrain tumor growth by |
| | | | | | | promoting local anti-tumor T cell |
| | | | | | | responses¹¹¹. |
| RET-GDNF | Epithelial | Immune | 0.43 | 0.10 | −0.33 | Endoneurial macrophages can induce |
| | | | | | | perineural invasion through GDNF-RET |
| | | | | | | signaling¹⁸⁵ |
| FAS-FASLG | Immune | Immune | 0.44 | −0.018 | −0.46 | Fas activation on immune cells, and in |
| | | | | | | particular cytotoxic lymphocytes, may |
| | | | | | | reduce antitumor efficacy¹⁸⁶ |
| IL10RA-IL10 | Immune | Immune | 0.0026 | 0.38 | 0.38 | Although IL10 is often thought of as a |
| | | | | | | potent anti-inflammatory cytokine¹⁸⁷, |
| | | | | | | recent evidence suggests that IL-10- |
| | | | | | | based interactions may metabolically |
| | | | | | | reprogram dysfunctional CD8⁺ T cells to |
| | | | | | | enhance anti-tumor immunity¹⁸⁸ |

The lower expression of the squamoid program in post-treatment residual malignant cells was unexpected, as prior studies have associated squamous/basal-like tumors with poor treatment response and outcomes^{6,8-10} and the squamoid program was associated with poor prognosis in Applicant's analysis of bulk profiles from untreated patients (FIG. 3B, 3C, 3E). However, prior classification based on untreated tumors did not account for potential reprogramming of expression profiles after treatment. Furthermore, previous reports have suggested that the basal-like A phenotype, distinguished by higher expression of squamous differentiation programs, is enriched in metastatic disease, which offers a potential explanation for the poor prognosis associated in Applicant's analyses⁶⁹. Applicant did not observe a significant depletion of the classical and squamoid programs in the DSP data (FIG. 3B; FIG. 21A-21B), but this may have been due to selection of tumors with poor treatment response to facilitate recovery of ROIs with adequate cancer cells.

The post-treatment enrichment and depletion of specific malignant and CAF programs may result from selection of pre-existing phenotypes and/or treatment-induced plasticity. The presence of the neural-like progenitor and neuroendocrine-like phenotypes in untreated specimens, albeit at lower prevalence, and the monotonic increase in the neural-like progenitor program (and depletion of the classical and squamoid programs) with increasing treatment response support a model wherein treatment-mediated selection of pre-existing phenotypes shapes residual disease (FIG. 2A, 3A-3C). These patterns are also present when comparing tumors with poor treatment response and abundant residual disease to those from untreated patients, suggesting a potential role for phenotypic plasticity (FIG. 3C). Future studies comparing matched pre- and post-treatment specimens and preclinical models with genetic tracing¹³² should provide further insights.

In conclusion, Applicant's high-resolution molecular framework sheds light on the inter- and intra-tumoral diversity of pancreatic cancer; spatial organization into discrete communities; treatment-associated remodeling; and clinically-relevant prognostication. These findings can be harnessed to augment precision oncology efforts in pancreatic cancer.

### Methods

### Human Patient Specimens

For inclusion in this study, patients had non-metastatic pancreatic ductal adenocarcinoma and went to surgical resection with or without prior neoadjuvant treatment in the form of chemotherapy and radiotherapy. An institutional clinical standard for grading neoadjuvant therapy response in surgical pathology was employed with the following scale: score of 1 is “moderate response”; 2 is “minimal response”; and 3 is “poor response.” Most treated patients received several cycles of FOLFIRINOX chemotherapy (5-FU, leucovorin, irinotecan, oxaliplatin) followed by multi-fraction radiotherapy with concurrent capecitabine or 5-FU. Four patients received other forms of chemotherapy such as cisplatin, gemcitabine, or nab-paclitaxel. Seven patients received additional neoadjuvant therapy in the form of losartan, an angiotensin II receptor type 1 antagonist, and/or nivolumab, a PD-1 inhibitor, on two clinical trials (NCT03563248, NCT01821729). The most common radiotherapy regimens included 30 Gy in 10 fractions, 50.4 Gy in 28 fractions (with dose painting up to 58.8 Gy to cover high-risk areas such as tumor-vessel interfaces), and stereotactic body radiotherapy 36 Gy in 6 fractions (with dose painting up to 42 Gy to cover high-risk areas such as tumor-vessel interfaces). Conformal techniques, most commonly volumetric modulated arc therapy, were employed for treatment delivery.

All patients were consented to excess tissue biobank protocol 2003P001289 (principal investigator: CFC; co-investigators: ASL, WLH), which was reviewed and approved by the Massachusetts General Hospital (MGH) Institutional Review Board. Resected primary tumor samples were examined to confirm neoplastic content by a board-certified pathologist (MMK) and then snap frozen and stored at −80° C. for up to 5 years prior to processing. Specimens were screened for an RNA integrity number (RIN; Agilent RNA 6000 Pico Kit, cat. no. 5067-1513) greater than an empirically determined threshold of 6; only specimens with RIN>6 were processed further. In many cases, matched formalin-fixed paraffin-embedded (FFPE) blocks were used for multiplexed ion beam imaging (MIBI, Ionpath) and digital spatial profiling (DSP, Nanostring).

### Organoid Derivation and Ex Vivo Chemoradiotherapy

Fresh patient-derived tumor tissue was minced with a razor blade in 1× PBS and incubated in digestion buffer (125 U/mL collagenase IV in 1x PBS; Worthington, cat. no. LS004189) for 30 minutes at 37° C. with constant agitation in a hybridization oven. Tumor cell suspension was poured over a 70 μm filter, washed with 1× PBS, and centrifuged at 500×g with slow deceleration. Cell pellets were resuspended in 85% growth-factor reduced Matrigel (Corning, cat. no. 356231) and 15% complete media (see below for details), plated as 50 μL plugs in a 24-well plate, and solidified at 37° C. Cells were cultured in complete media, monitored for outgrowth, and passaged with TrypLE Express (Life Technologies) for four passages to purify the malignant epithelial component from contaminating stromal cells.

Organoids were then subjected to a 10-day ex vivo chemoradiotherapy regimen as follows. Organoids were plated in complete media. Twenty-four hours later, organoids were treated with FOLFIRINOX-like chemotherapy (SN-38 substituted for irinotecan) for four days using a molar ratio similar to that given to patients (molar ratio of SN-38 adjusted to account for enhanced activity relative to irinotecan)^{145,146}. Chemotherapy consisted of 34.4 μm 5-fluorouracil (Sigma-Aldrich, cat. no. F6627), 4 nm SN-38 (Sigma-Aldrich, cat. no. H0165-10 MG), and 0.32 μm oxaliplatin (Sigma-Aldrich, cat. no. 09512). Complete media and drugs were replaced on Day 3. Chemotherapy was terminated on Day 5. On Day 5 and Day 7, radiotherapy (7.5 Gy) was administered using a dual ¹³⁷Cs source irradiator (Gammacell 40 Exactor, Best Theratronics). After three days of rest, organoid plugs were flash frozen and nucleus isolation was performed using the same protocol as tumor specimens as described below. Matched pre- and post-treatment organoids were compared by snRNA-seq.

Complete media for pancreatic organoids was formulated based on L-WRN cell conditioned media (L-WRN CM) as described previously¹⁴⁷⁻¹⁴⁹. Briefly, L-WRN CM was generated by collecting eight days of supernatant from L-WRN cells, grown in Advanced DMEM/F12 (Gibco) supplemented with 20% fetal bovine serum (Hyclone), 2 mM GlutaMAX, 100 U/mL of penicillin, 100 mg/mL of streptomycin, and 0.25 mg/mL amphotericin. L-WRN CM was diluted 1:1 in Advanced DMEM/F12 (Gibco) and supplemented with additional RSPO-1 conditioned media (10% v/v), generated using Cultrex HA-R-Spondin1-Fc 293T cells. Complete media were supplemented with the following additives: B27 (Gibco), 1 mM N-acetylcysteine (Sigma-Aldrich), 10 mM nicotinamide (Sigma-Aldrich), 50 ng/mL EGF (Novus Biologicals), 500 nM A83-01 (Cayman Chemical), 10 mM SB202190 (Cayman Chemical), and 500 nM PGE2 (Cayman Chemical). Wnt activity of the conditioned media was assessed and normalized between batches via luciferase reporter activity of TCF/LEF activation (Enzo Leading Light Wnt reporter cell line).

Nucleus Isolation from Frozen Samples

Applicant has recently published a toolbox for snRNA-seq of tumors spanning a broad range of nucleus isolation techniques for various tissue/tumor types³⁷, but not PDAC. The following protocol is an adaptation and optimization of this prior work specifically for the unique tissue requirements of pancreatic tumors. A 2× stock of STc buffer in nuclease-free water was prepared with a final concentration of 292 mM NaCl (ThermoFisher Scientific, cat. no. AM9759), 40 mM Tricine (VWR, cat. no. E170-100G), 2 mM CaCl₂) (VWR, cat. no. 97062-820), and 42 mM MgCl₂ (Sigma Aldrich, cat. no. M1028). For each specimen, 2 mL of NSTcPA buffer was prepared by combining 1 mL of 2× STc buffer, 40 μL of 10% Nonidet P40 Substitute (Fisher Scientific, cat. no. AAJ19628AP), 10 μL of 2% bovine serum albumin (New England Biolabs, cat. no. B9000S), 0.3 L of IM spermine (Sigma-Aldrich, cat. no. S3256-1G), 1 μL of IM spermidine (Sigma-Aldrich, cat. no. S2626-1G), and 948.7 μL of nuclease-free water. For each specimen, 3 mL of 1x working STc buffer was made by diluting 2x STc 1:1 in nuclease-free water.

NSTcPA buffer (1 mL) was pipetted into one well of a 6-well plate (Stem Cell Technologies, cat. no. 38015) on ice. The frozen tumor specimen was removed from −80° C. and placed in a petri dish on dry ice. Using a clean razor blade, the desired regions of the tissue were cut on dry ice so the specimen remained frozen. The amount of each tumor processed for snRNA-seq varied but was typically 20-50 mg; fragments from several regions of the tumor were processed together to reduce spatial sampling bias. The remainder of the specimen was returned to −80° C. for subsequent use. The selected tissue was transferred into the NSTcPA buffer and manually minced with fine straight tungsten carbide scissors (Fine Science Tools, cat. no. 14568-12) for 8 minutes. The homogenized tissue solution was then filtered through a 40 μm Falcon cell filter (Thermo Fisher Scientific, cat. no. 08-771-1) into a 50 mL conical tube. An additional 1 mL of NSTcPA buffer was used to rinse the well and filter. The total volume was brought up to 5 mL with 3 mL of 1× STc buffer and transferred into a 15 mL conical tube. The sample was spun for 5 min at 500×g, 4° C. and the supernatant was removed. The pellet was resuspended in 100-200 μL 1× STc and then filtered through a 35 μm Falcon cell strainer (Corning, cat. no. 352235). Nuclei were quantified using a C-chip disposable hemocytometer (VWR, cat. no. 82030-468) and diluted in 1× STc as necessary to achieve a final concentration of 300-2,000 nuclei/μL.

Single-Nucleus RNA-Seq (snRNA-Seq)

Approximately 8,000-10,000 nuclei per sample were loaded into each channel of a Chromium single-cell 3′ chip (V2 or V3, 10× Genomics) according to the manufacturer's instructions. Single nuclei were partitioned into droplets with gel beads in the Chromium Controller to form emulsions, after which nuclear lysis, barcoded reverse transcription of mRNA, cDNA amplification, enzymatic fragmentation, and 5′ adaptor and sample index attachment were performed according to manufacturer's instructions. Up to four sample libraries were sequenced on the HiSeq X Version 2.5 (Illumina) with the following paired end read configuration: read 1, 26-28 nt; read 2, 96-98 nt; index read, 8 nt.

snRNA-Seg Data Pre-Processing

BCL files were converted to FASTQ using bcl2fastq2-v2.20. CellRanger v3.0.2 was used to demultiplex the FASTQ reads, align them to the hg38 human transcriptome (pre-mRNA) reference and extract the UMI and nuclei barcodes. The output of this pipeline is a digital gene expression (DGE) matrix for each sample, which has quantified for each nucleus barcode the number of UMIs that aligned to each gene.

Applicant filtered low-quality nuclei profiles by baseline quality control measures including number of reads captured and ambient RNA detection. First, Applicant used CellBender remove-background¹⁵⁰ to remove ambient RNA, enhancing cell distinction and marker specificity. CellBender remove-background was run (on Terra) to remove ambient RNA and other technical artifacts from the count matrices. The workflow is available publicly as cellbender/remove-background (snapshot 11) and documented on the CellBender github repository as v0.2.0: https://github.com/broadinstitute/CellBender. This latest version of CellBender remove-background cleans up count matrices using a principled model of noise generation in scRNA-Seq. The parameters “expected-cells” and “total-droplets-included” were chosen for each dataset based on the total UMI per cell vs. cell barcode curve in accordance with CellBender documentation. Other inputs were left at their default values. The false positive rate parameter “fpr” was set to 0.01, 0.05, and 0.1. For downstream analyses Applicant used the ‘FPR_0.01_filtered.h5’ file. Following this step, Applicant filtered out nuclei with over 10,000 UMI counts. To account for differences in sequencing depth across nuclei, UMI counts were normalized by the total number of UMIs per nucleus and converted to transcripts-per-10,000 (TP10K) as the final expression unit.

### Dimensionality Reduction, Clustering and Annotation

Following these quality control steps, treatment-naïve and neoadjuvant-treated specimens were aggregated into a single joint dataset. The log₂(TP10K+1) expression matrix was constructed for the following downstream analyses. Applicant identified the top 2,000 highly variable genes across the entire dataset using the Scanpy¹⁵¹ highly variable genes function with the sample ID as input for the batch. Applicant then performed a Principal Component Analysis (PCA) over the top 2,000 highly variable genes and identified the top 40 principal components (PCs) beyond which negligible additional variance was explained in the data (the analysis was performed with 30, 40, and 50 PCs and robust to this choice). Subsequently, Applicant performed batch correction using Harmony-Pytorch¹⁵² and built a k-nearest neighbors graph of nuclei profiles (k=10) based on the top 40 batch corrected components and performed community detection on this neighborhood graph using the Leiden graph clustering method¹⁵³ with resolution set to 1 to identify distinct cell population clusters. Individual nucleus profiles were visualized using the Uniform Manifold Approximation and Projection (UMAP)¹⁵⁴. Distinct cell populations identified from the previous steps were annotated using known cell type-specific gene expression signatures^{34,44-47}. Representative marker genes included but were not limited to: ductal (CFTR), malignant epithelial (KRT6A, KRT7, KRT14, KRT17, KRT19, TACSTD2, S100A11, S100A16, TFF1, and CLDN18), acinar (CPB1, PRSS3, AMYIA), acinar-REG+ (REG3A, REG3G, REG1B), cancer-associated fibroblast (COLIA1, FN1, PDPN, DON, VIM, FAP, ACTA2), vascular smooth muscle (MYH11, MYOCD)), pericyte (PDGFRB, DLK1, ACTA1, RGS5, CSPG4, MCAM), endothelial (PECAM1, VWF), vascular endothelial (ESAM, FLT1, EPAS1), lymphatic endothelial (FLT4, SEMA3A, SEMA3D)), adipocyte (PLIN1, LPL), alpha (GCG), beta (INS, IAPP), delta (SST), gamma (PPY), epsilon (GHRL), neuroendocrine (SYP, (HGA), intrapancreatic neurons (TH, (HAT, ENO2, NSE), Schwann (SOX10, S100B), pan-immune (PTPRC), antigen-presenting cell (CD74), macrophage (CD68, CD163, MRC1, CD80, CD86, TGFB1, CSF1), monocyte (TLR2, ITGB2, ITGAM, CTSD, CTSA, NLRP3, CLEC7A, BST1, STAB1, IRAK3), eosinophil (MBP, EDN, EPO, CCR3), CDC1 (XCR1, CST3, CLEC9A, LGALS2), cDC2 (CD1A, CD207, CD1E, FCER1A, NDRG2), activated DC (FSCN1, LAMP3, (CL19, (CR7), plasmacytoid DC (GZMB, IRF7, LILRA4, TCF4, CXCR3, IRF4), T cell (CD4, CD8A, CD8B, CD3D, THEMIS, CD96, IKZF1, GZMA, FOXP3), B cell (BANK1, (D19), NK cell (KLRD1, KIR2DL3, IL18R1, KIR2DL1, KIR3DL2), plasma (SDC1, IGLC2), mast (CPA3, KIT), neutrophil (CSF3R, CXCL8). The Adjusted Mutual Information (AMI) score measures the consistency between two partitions over all cells. Applicant used the AMI to quantify the similarity in single cell assignments between the partitions imposed by the Leiden clustering labels and patient ID labels. The AMI was computed using the adjusted_mutual_info_score function in the Python sklearn package.

While earlier scRNA-seq studies in PDAC did not fully capture the stromal milieu and necessitated enrichment strategies for CAFs such as fluorescence-activated cell sorting⁴⁸⁻⁵¹, they were well-represented in the samples. Specifically, Applicant's snRNA-seq had a higher yield of high quality nuclei per patient in the untreated group (6,054±1,529) than a recent scRNA-seq study of primary untreated PDAC⁵¹ (1,718±773), despite comparable quantities of loaded cells/nuclei (p=1.92×10⁻⁹, Mann-Whitney U test; FIG. 24), recovered six additional cell types absent in scRNA-seq, and captured significantly higher proportions of CAFs, pericytes, and endocrine cells and lower proportions of vascular smooth muscle cells, myeloid cells, lymphoid cells, and endothelial cells (p<0.05; Mann-Whitney U Test; comparable results using Dirichlet-multinomial regression; FIG. 24).

### Quantifying Statistically Significant Changes in Composition Between Cell Populations

To compute the significance of changes in the cellular composition between untreated and treated (CRT and CRTL) samples, Applicant used multiple statistical tests that each capture different types of information: (1) Dirichlet-multinomial regression, and (2) Mann-Whitney test. To account for dependencies among cell proportions (an increase in the proportion of one cell subset will necessarily lead to a decrease in the proportions of the other cell subsets), Applicant used a Dirichlet-multinomial regression. This statistical test and its inclusion probabilities (pᵢ) were calculated using the “scCODA” Python package¹⁵⁵. Applicant also performed a non-parametric Mann-Whitney U test (two-sided) on the proportions of each cell subset in untreated versus treated (CRT and CRTL) samples. Bonferroni corrections were applied in instances where multiple pairwise comparisons were made between treatment or response groups. These same statistical approaches were applied to quantify the differences in cells/nuclei captured by the snRNA-seq approach and a previously published scRNA-seq method⁵¹.

Inferring Copy Number Aberrations from Single Nucleus Profiles

A Python implementation of InferCNV v3.9 based on the InferCNV R implementation as provided at https://github.com/broadinstitute/inferCNV (inferCNV of the Trinity CTAT Project) was run jointly on all treated and untreated single nuclei profiles. To avoid circularity, Applicant used a set of high confidence non-neoplastic cells as the reference that was derived from two non-malignant pancreas snRNA-seq samples. Applicant used the default parameters for InferCNV including a 100-gene window in sub-clustering mode and a hidden Markov model to predict the copy number aberration (CNA) count and construct a CNA score for each nucleus based on the predicted CNAs in each cell. Annotated epithelial cells were subject to Leiden sub-clustering and those with an average CNA score greater than 0.01 were labeled as malignant epithelial cells.

### Partition-Based Graph Abstraction

The pseudotemporal orderings/trajectories of annotated epithelial cell types was estimated using the diffusion map and partition-based graph abstraction (PAGA v1.2) method⁶⁷. The diffusion map was computed with 15 components and the cell neighborhood map utilized a local neighborhood of 15.

### Multiplexed Ion Beam Imaging (MIBI)

Formalin-fixed paraffin-embedded pancreatic tissue sections were cut onto gold MIBI slides (IONpath, cat. no. 567001) and stained at IONpath (Menlo Park, CA) with the internal Epithelial i-Onc isotopically-labelled antibody panel (IONpath): dsDNA_89 [3519 DNA] (1:100), β-tubulin_166 [D3U1W] (3:200), CD163_142 [EPR14643-36] (3:1600), CD4_143 [EPR6855] (1:100), CD11c_144 [EP1347Y] (1:100), LAG3_147 [17B4] (1:250), PD-1_148 [D4W2J] (1:100), PD-L1_149 [EIL3N] (1:100), Granzyme B_150 [D6E9W] (1:400), CD56_151 [MRQ-42] (1:1000), CD31_152 [EP3095] (1:1000), Ki-67_153 [D2H10] (1:250), CD11b_155 [D6XIN] (1:500), CD68_156 [D4B9C] (1:100), CD8_158 [C8/144B] (1:100), CD3_159 [D7A6E] (1:100), CD45RO_161 [UCHL1] (1:100), Vimentin_163 [D21H3] (1:100), Keratin_165 [AE1/AE3] (1:100), CD20_167 [L26] (1:400), Podoplanin_170 [D2-40] (1:100), IDO1_171 [EPR20374] (1:100), HLA-DR_172 [EPR3692] (1:100), DC-SIGN_173 [DCN46] (1:250), CD45_175 [2B11 & PD7/26] (3:200), HLA class 1 A, B, and C_176 [EMR8-5] (1:100), Na/K-ATPase_176 [D4Y7E] (1:100).

Quantitative imaging was performed using a beta unit MIBIscope (IONpath) equipped with a duoplasmatron ion source. This instrument sputters samples with O2 primary ions line-by-line, while detecting secondary ions with a time-of-flight mass spectrometer tuned to 1-200 m/z+ and mass resolution of 1000 m/Am, operating at a 100 KHz repetition rate. The primary ion beam was aligned daily to minimize imaging astigmatism and ensure consistent secondary ion detection levels using a built-in molybdenum calibration sample. In addition to the secondary ion detector, the MIBIscope is equipped with a secondary electron detector which enables sample identification and navigation prior to imaging.

For data collection, three fields of view were acquired for each sample by matching the secondary electron morphological signal to annotated locations on sequential H&E stained slides. The experimental parameters used in acquiring all imaging runs were as follows: pixel dwell time (12 ms), image size (500 μm² at 1024×1024 pixels), primary ion current (5 nA O₂⁺), aperture (300 μm), stage bias (+67 V).

### MIBI Image Processing, Segmentation and Quantification

Mass spectrometer run files were converted to multichannel tiff images using MIB.io software (IONpath). Mass channels were filtered individually to remove gold-ion background and spatially uncorrelated noise. HLA Class 1 and Na/K-ATPase signals were combined into a single membrane marker. These image files (tiff) were used as a starting point for single cell segmentation, quantification, and interactive analysis using histoCAT (v1.76)¹⁵⁶. Applicant followed a similar approach for segmentation as proposed for Imaging Mass Cytometry data¹⁵⁶⁻¹⁵⁸. Briefly, Applicant used Ilastik¹⁵⁹ to manually train three classes (nuclei, cytoplasm and background) to improve subsequent watershed segmentation using CellProfiler¹⁶⁰. Finally, the tiff images and masks were combined for histoCAT loading with a script optimized for MIBI image processing (code, classifiers and configuration files are available at https://github.com/DenisSch/MIBI).

Immune cells were further partitioned into cell subsets by incorporating the full set of protein markers available along with the untreated and treated snRNA-seq data. First, Applicant used the gim VI variational autoencoder to train a model¹⁶¹ taking both spatial MIBI and snRNA-seq data modalities as well as the correspondence between genes and antibody markers as input and encoding both the MIBI and snRNA-seq datasets into a joint latent space. The gim VI model was trained for 10 epochs. The latent space representation of the snRNA-seq data was then extracted from this model and used as the features to build a random forest model for cell type classification. Subsequently, the latent space representation extracted for each MIBI image was then evaluated using Applicant's trained model to generate a predicted cell type for each segmented immune cell in the spatial data.

### Differential Gene Expression Analysis

For each annotated cell type detected in both untreated and treated tumors, a differential gene expression analysis using a mixed effects Poisson model was performed between cells in the two populations to identify upregulated and downregulated genes. Applicant considered untreated, all treated, CRT, and CRTL treatment categories in this analysis. Applicant constructed a mixed effects model with the sample ID as a random effect; treatment status, two principal components and sex were fixed effect covariates; and finally, the log total UMIs as an offset. The mixed effects model was implemented using the glmer R package¹⁶².

### Scoring Gene Signatures for Each Nucleus Profile and Patient

A signature score for each nucleus profile was computed as the mean log₂(TP10K+1) expression across all genes in the gene signature. Subsequently, to identify statistically significant gene expression patterns, Applicant computed the mean log₂(TP10K+1) expression across a background set of 50 genes randomly selected with matching expression levels to those of the genes in the signature iterated 25 times. The gene signature score was defined to be the excess in expression found across the genes in the signature compared to the background set. To score gene programs at the patient level, these gene program scores were normalized for each nucleus and then the mean of all nuclei from an individual tumor was computed for each program of interest.

### Consensus Non-Negative Matrix Factorization

Applicant formulated the task of dissecting gene expression programs as a matrix factorization problem where the input gene expression matrix is decomposed into two matrices. The solution to this formulation can be identified by solving the following minimization problem:

$$arg min \{ \frac{1}{2} {‖ {X}_{n , m} - {W}_{n , p} \times {H}_{p , m} ‖}_{F}^{2} + ( 1 - α ) \frac{1}{2} ‖ {W}_{n , p} ‖ + \frac{1}{2} ( 1 - α ) ‖ {H}_{n , p} ‖ + α {‖ vec ( {W}_{n , p )} ‖}_{1} + α {‖ vec ( {H}_{n , p} ) ‖}_{1} \}$$

Applicant utilized the non-negative matrix factorization implemented in sklearn to derive the malignant and CAF expression programs across both untreated and treated samples. Because the result of NMF optimization can vary between runs based on random seeding, Applicant repeated NMF 50 times per cell type category and computed a set of consensus programs by aggregating results from all 50 runs and computed a stability and reconstruction error. This consensus NMF was performed by making custom updates to the cNMF python package⁷³. To determine the optimal number of programs (p) for each cell type and condition, Applicant struck a balance between maximizing stability and minimizing error of the cNMF solution, while ensuring that the resulting programs were as biologically coherent and parsimonious as possible. Each program was annotated utilizing a combination of GSEA¹⁶³ and comparison to bulk expression signatures.

### Measuring Similarity Between Gene Expression Programs

To measure the similarity between cNMF-derived gene expression programs and pre-existing bulk derived gene sets representing PDAC subtypes or differentially-expressed genes associated with perineural invasion, Applicant performed the hypergeometric test and Kolmogorov-Smirnov test, respectively, to quantify the overlap between the two gene sets. This test enables us to determine enrichment or depletion of gene expression programs in a pre-defined gene set. To measure the similarity among the cNMF-derived gene expression programs, Applicant computed the correlation of the cell by program vector for each program to identify which programs were found to be co-occurring across the same cells. Finally, Applicant computed the patient-level statistical comparisons of program compositional changes by treatment type and response. This were performed by computing the average program weight over all cells for each patient and testing for changes to the program abundance using statistical tests as described in the prior section on quantifying statistically significant changes in cell composition between cell populations.

### Multiplexed Immunofluorescence Validation of the Neural-Like Progenitor Program

For multiplexed immunofluorescence validation of the neural-like progenitor program, Applicant prepared an FFPE section from an independent PDAC tumor in the same manner as the DSP experiments described above except that probe hybridization and subsequent washes were omitted. Applicant incubated the slide with 1:10 SYTO13 (ThermoFisher Scientific, cat. no. 57575), 1:40 anti-panCK-Alexa Fluor 532 (clone AE-1/AE-3; Novus Biologicals, cat. no. NBP2-33200AF532), and 1:50 anti-NRXN3 (rabbit polyclonal IgG; Invitrogen, cat. no. PA5-101708) in blocking buffer W (NanoString) for 1 hour at room temperature followed by secondary antibody staining with 1:1000 goat anti-rabbit IgG Alexa Fluor 647 (Invitrogen, A-21245) for 1 hour at room temperature. The slide was imaged on the NanoString GeoMx instrument in slide scanning mode with exposure times of 150 ms, 600 ms, and 75 ms in the SYTO 13, Alexa Fluor 532, and Alexa Fluor 647 channels, respectively.

### Survival Analysis of Bulk RNA-Seq Data

Bulk RNA-seq data from two previously published resected primary PDAC cohorts with overall survival annotated were obtained (The Cancer Genome Atlas, n=139; PanCuRx, n=168) 11.14.69. Patients with metastases or those that received neoadjuvant therapy were excluded from this analysis, yielding a total of 266 patients for further analysis. Gene expression levels from RNA-seq data was estimated using RSEM164.

To score malignant and fibroblast cNMF programs in each tumor, Applicant summed the expression of the top 200 genes for each program and z-score normalized the expression scores within the TCGA and PanCuRx cohorts independently to account for batch effects. Age, sex, grade and stage were available for all patients. There were 154 progression events and 167 deaths. Since there were four clinicopathologic covariates and 18 gene expression programs across the malignant and CAF compartments, Applicant sought to consolidate some of the GEPs into aggregate programs to avoid overfitting a Cox proportional-hazards regression model. Towards this end, Applicant noted that among the malignant cell state programs, the TNF-NFκB, adhesive, interferon, and ribosomal programs featured 7-17% secreted proteins while the cycling(S), cycling (G2/M), and MY (′ programs exhibited 1% or fewer secreted proteins⁹¹. This allowed us to aggregate the seven malignant cell state programs into two binary categories: cycling (cycling(S), cycling (G2/M), MYC) and secretory (INF-NFκB, interferon, adhesive, ribosomal), yielding a total of 17 covariates in the Cox proportional-hazards regression model. Multivariable Cox regression analyses was performed for time to progression (TTP) and overall survival (OS) (Stata/SE 15.1).

### Digital Spatial Profiling Experiments

Applicant followed published experimental methods⁴³ (Nanostring) with modifications as noted below. Briefly, serially sectioned formalin-fixed paraffin-embedded (FFPE) sections (5 μm) of 21 specimens were prepared by the MGH Histopathology Core on the IRB-approved protocol (2003P001289) to generate consecutive sections that were processed for H&E and WTA, respectively. For WTA, slides were baked at 60° C. for one hour, deparaffinized with CitriSolv (DECON), rehydrated, antigen-retrieved in 1x Tris-EDTA/pH 9 in a steamer for 15 min at 100° C., proteinase K (ThermoFisher Scientific, AM2548) digested at 0.1 ng/mL for 15 min at 37° C., post-fixed in neutral-buffered formalin for 10 min, hybridized to UV-photocleavable barcode-conjugated RNA in situ hybridization probe set (WTA with 18,269 targets) overnight at 37° C., washed to remove off-target probes, and then counterstained with morphology markers for 2 hours at room temperature.

The morphology markers consisted of: 1:10 SYTO13 (ThermoFisher Scientific, cat. no. 57575), 1:20 anti-panCK-Alexa Fluor 532 (clone AE-1/AE-3; Novus Biologicals, cat. no. NBP2-33200AF532), 1:100 anti-CD45-Alexa Fluor 594 (clone D9M8I; Cell Signaling Technology, cat. no. 13917S), and 1:100 anti-αSMA-Alexa Fluor 647 (clone 1A4; Novus Biologicals, cat. no. IC1420R) in blocking buffer W (NanoString). The anti-panCK and anti-αSMA antibodies were acquired pre-conjugated whereas the anti-CD45 antibody was conjugated using the Alexa Fluor 594 Antibody Labeling Kit (Invitrogen, A20185). These four morphology markers allowed delineation of the nuclear, epithelial, immune, and fibroblast compartments. Immunofluorescence images, region of interest (ROI) selection, segmentation into marker-specific areas of interest (AOI), and spatially-indexed barcode cleavage and collection were performed on a GeoMx Digital Spatial Profiling instrument (NanoString). Typical exposure times were 50 ms for SYTO13, 300 ms for anti-panCK-Alexa Fluor 532, 400-450 ms for anti-CD45-Alexa Fluor 594, and 50 ms for anti-αSMA-Alexa Fluor 647. Approximately 8-14 ROIs and 20-36 AOIs were collected per specimen. Library preparation was performed according to the manufacturer's instructions and involved PCR amplification to add Illumina adapter sequences and unique dual sample indices. A minimum sequencing depth of 150-200 reads per square micron of illumination area was achieved by sequencing all WTA AOIs on a NovaSeq S2 (100 cycles, read 1:27 nt, read 2:27 nt, index 1:8 nt, index 2:8 nt).

### Digital Spatial Profiling-Data Preprocessing

FASTQ files for DSP were aggregated into count matrices as described previously43. Briefly, deduplicated sequencing counts were calculated based on UMI and molecular target tag sequences. Single probe genes were reported as the deduplicated count value. The limit of quantitation (LOQ) was estimated as the geometric mean of the negative control probes plus 2 geometric standard deviations of the negative control probes. Targets were removed that consistently fell below the LOQ, and the datasets were normalized using upper quartile (Q3) normalization. Normalized expression was detrended to model cell-type specific expression by calculating an adjustment factor:

$${A}_{S 1 , g , r} = {E}_{S 1 , g , r} * ( {E}_{S 1 , g , r} - max ( {E}_{S 2 , g , r} {E}_{S 3 , g , r} ) )$$

Where adjustment factors, A, are calculated for the expression, E, of a gene, g, within a given ROI, r, by comparing a given segment, SI, to the max expression observed in other segments, S2 and S3. The original expression was then detrended by calculating:

$${D}_{S 1 , g , r} = {E}_{S 1 , g , r} - ( \frac{2 * {E}_{S 1 , g , r}}{max ( {E}_{S 1 , r} )} ) * {log}_{2} ( max ( {A}_{S 1 , g , r} 1 ) )$$

This resulted in detrended expression, D, reflecting the original expression minus positive adjustment factors scaled based on the relative expression of the target to all other targets in the segment.

### Digital Spatial Profiling-Program Scoring and Correlation Analysis

Statistical analysis was performed using R. Programs were scored for each DSP sample within each region of interest using single-sample gene set enrichment analysis (ssGSEA)¹⁶⁵, which were transformed using the z-score. For each program, intra-patient dispersion of program expression across ROIs was calculated as the patient-level mean of the interquartile range (IQR; difference between upper and lower quartiles) across all ROIs within each individual tumor:

$$\frac{{\sum }_{j = 1}^{n} 1 {QR}_{i = 1}^{{r}_{j}} ( {p}_{i , j} )}{n}$$

where n=number of patients; rⱼ=number of ROIs in patient j; and p_{i,j}=program score for ROI i in patient j. In contrast, inter-patient dispersion of program expression was computed as the IQR of the mean program score for each tumor:

$${IQR}_{j = 1}^{n} \{ \frac{{\sum }_{i = 1}^{{r}_{j}} {p}_{i , j}}{{r}_{j}} \} .$$

Unsupervised hierarchical clustering was performed on all features (malignant programs, CAF programs, deconvolved immune cell type proportions, compartment areas within ROI) using the Pearson correlation distance and average linkage. Cell deconvolution analysis was performed using the SpatialDecon package (https://github.com/Nanostring-Biostats/SpatialDecon/). Analysis of expression or program scores used linear mixed effects models¹⁶⁶ to control for multiple sampling within a slide, using Satterthwaite's approximation¹⁶⁷ for degrees of freedom for p-value calculation. Correlation coefficients were calculated using the Spearman rank correlation.

### Digital Spatial Profiling—Receptor Ligand (RL) Correlation Across ROIs

Known receptor-ligand pairs were obtained from CellPhoneDB with potential receptor-ligand pairs quantified using the Spearman rank correlation between paired segments within the same ROI across all ROIs with said pairs. Interactions were calculated for non-self (juxtacrine) and self (autocrine) occurring within the same segment. Receptor-ligand interactions were calculated separately for untreated and CRT specimens to determine interactions that are differential between conditions. All analyses were two-sided and used a significant level of p-value≤0.05 and were adjusted for multiple testing where appropriate using the false discovery rate¹⁶⁸. Tables 7A-7B shows spearman rank correlation coefficients for receptor-ligand pairs (CellPhoneDB) expressed in all paired segments (epithelial, CAF, immune) within the same ROI across all ROIs with said segment pairs, stratified by treatment group (CRT vs. untreated).

TABLE 7A
LR Untreated

| | Immune)- | Immune)- | Immune)- | CAF)- | CAF)- | CAF)- | Epithelial)- | Epithelial)- | Epithelial)- |
|---|---|---|---|---|---|---|---|---|---|
| | CAF | Epithelial | Immune | Epithelial | Immune | CAF | Immune | CAF | Epithelial |
| SEMA3F)- | −0.00550824 | −0.083627 | 0.1739249 | 0.08253465 | 0.06441402 | 0.07392476 | 0.01435107 | 0.042488 | 0.12446264 |
| PLXNA3 | | | | | | | | | |
| SEMA3F)- | 0.09333224 | 0.09765941 | 0.08312037 | 0.18671057 | 0.11999292 | 0.00171876 | 0.07281945 | 0.08018374 | 0.26735897 |
| PLXNA1 | | | | | | | | | |
| SEMA3F)- | 0.064244886 | 0.11185975 | −0.0886181 | 0.07754854 | −0.0187306 | −0.0593438 | 0.07642256 | −0.2055834 | 0.38479847 |
| NRP1 | | | | | | | | | |
| SEMA3F)- | −0.027691487 | 0.05112714 | 0.04117555 | 0.0787349 | 0.04097377 | −0.0014024 | 0.04603136 | −0.1534901 | 0.36767299 |
| NRP2 | | | | | | | | | |
| HEBP1)- | 0.093388667 | 0.03423287 | −0.0706667 | −0.0534143 | 0.05462704 | −0.1131577 | 0.09753768 | 0.14435825 | −0.2158724 |
| ADRA2A | | | | | | | | | |
| HEBP1)- | −0.078181756 | −0.0867449 | 0.02804722 | 0.02374748 | −0.0425257 | 0.08197409 | −0.0110195 | −0.0049243 | −0.0196793 |
| FPR3 | | | | | | | | | |
| DCN)- | 0.034591057 | −0.1601603 | 0.08317318 | 0.05844382 | −0.0626026 | −0.1623714 | 0.11886235 | 0.05513417 | −0.2202065 |
| EGFR | | | | | | | | | |
| DCN)- MET | 0.27148856 | 0.02560242 | 0.20387491 | −0.0902288 | −0.2954851 | −0.2641571 | 0.32375445 | 0.32034121 | −0.0255966 |
| GRN)- | 0.198251694 | 0.16985139 | 0.2043561 | 0.11764172 | 0.07782545 | 0.10188786 | −0.0123767 | −0.0083664 | 0.01101425 |
| TNFRSF1A | | | | | | | | | |
| GRN)- | −0.013019887 | 0.1067557 | −0.0880148 | 0.03459296 | 0.00464792 | 0.0592212 | −0.1229404 | 0.02993575 | −0.0418314 |
| EGFR | | | | | | | | | |
| GRN)- | 0.15893265 | −0.1637907 | 0.34445724 | −0.1120615 | 0.03532425 | 0.03743993 | −0.1266349 | −0.0423757 | 0.08819028 |
| SORT1 | | | | | | | | | |
| GRN)- | −0.171705528 | −0.1760705 | 0.40212536 | −0.0201098 | −0.0567792 | 0.04416176 | −0.1075014 | −0.0550926 | −0.1060942 |
| TNFRSF1B | | | | | | | | | |
| GRN)- | −0.228503163 | −0.0735497 | 0.1441261 | −0.1783994 | −0.0023708 | −0.1805803 | −0.1897924 | −0.1589144 | −0.3792486 |
| CLEC4M | | | | | | | | | |
| ICAM3)- | −0.148089649 | −0.0803019 | 0.11151626 | 0.18999698 | −0.1159882 | 0.25518934 | −0.1026049 | 0.17955369 | 0.27984779 |
| ITGAL | | | | | | | | | |
| ICAM3)- | 0.004739688 | −0.003065 | −0.0182834 | 0.11787454 | −0.1377139 | 0.11131961 | −0.165786 | 0.10941516 | 0.17205378 |
| ITGB2 | | | | | | | | | |
| ICAM3)- | −0.059101665 | −0.0007596 | 0.13782697 | 0.08532636 | −0.0431598 | 0.31230998 | 0.21462651 | 0.04213794 | 0.28268238 |
| CLEC4M | | | | | | | | | |
| ICAM3)- | −0.033203785 | 0.04701419 | 0.23393833 | 0.07357256 | 0.09937005 | 0.0409783 | 0.16087016 | 0.05946994 | 0.20132174 |
| CD209 | | | | | | | | | |
| CEACAM1)- | 0.033662389 | −0.0157307 | 0.21821785 | −0.0486275 | 0.2432647 | 0.03281272 | −0.1964282 | 0.00217384 | 0.04020851 |
| EGFR | | | | | | | | | |
| CEACAM1)- | 0.055007701 | 0.13646303 | 0.14805293 | 0.09551373 | 0.05591367 | 0.05566983 | −0.0646539 | −0.3504923 | −0.3904767 |
| SELE | | | | | | | | | |
| CEACAM1)- | 0.122773249 | 0.06795762 | 0.08208764 | 0.07535408 | 0.00247089 | 0.08573869 | −0.234336 | −0.1409914 | −0.2749967 |
| CD209 | | | | | | | | | |
| ST6GAL1)- | −0.089346 | 0.17227869 | −0.1418657 | −0.0692167 | 0.09508156 | 0.13841581 | 0.25900438 | 0.19466849 | −0.2013953 |
| EGFR | | | | | | | | | |
| NTN1)- | 0.115692025 | 0.16585691 | 0.19715387 | 0.23873535 | 0.13607222 | 0.14235768 | 0.21583588 | 0.175786 | 0.47745708 |
| UNC5D | | | | | | | | | |
| NTN1)- | −0.103368348 | 0.05777222 | 0.06205157 | 0.20242188 | 0.16702487 | −0.0155862 | −0.0120828 | 0.14045202 | 0.03513166 |
| UNC5B | | | | | | | | | |
| NTN1)- | 0.014326396 | 0.10460422 | 0.16659361 | 0.10487175 | 0.20398475 | 0.16260704 | 0.07455021 | 0.10336901 | 0.38884121 |
| UNC5A | | | | | | | | | |
| NTN1)- | 0.106635606 | 0.16171236 | 0.23917297 | 0.12092511 | −0.0589022 | 0.26026896 | 0.11535132 | 0.19936926 | 0.30286493 |
| ADORA2B | | | | | | | | | |
| NTN1)- | −0.053143665 | −0.0881179 | 0.10129782 | 0.05279109 | −0.1192931 | 0.07582316 | 0.08658383 | −0.039198 | −0.024775 |
| NEO1 | | | | | | | | | |
| SCT)- | −0.070396594 | 0.1067248 | 0.31313136 | 0.11823712 | −0.058419 | 0.13618718 | 0.17591059 | 0.07318124 | 0.40814354 |
| PTH1R | | | | | | | | | |
| SCT)- | 0.060762016 | 0.08992363 | 0.14418596 | 0.11191376 | 0.14854975 | 0.01410346 | 0.1382846 | 0.05367493 | 0.40880642 |
| RAMP2 | | | | | | | | | |
| SCT)- | −0.008016728 | −0.0721269 | 0.09146465 | 0.00198098 | −0.0369382 | −0.0349828 | −0.0517197 | 0.19328763 | 0.05037298 |
| VIPR1 | | | | | | | | | |
| SCT)- | 0.047518566 | 0.07216465 | 0.25040264 | 0.10459009 | −0.0197397 | 0.15826058 | 0.04865231 | 0.07166716 | 0.38260857 |
| ADRB2 | | | | | | | | | |
| SCT)- | −0.103083825 | 0.04608997 | 0.23657381 | 0.09620612 | 0.22001659 | −0.12823 | 0.18706735 | −0.0745701 | 0.41266056 |
| ADRB3 | | | | | | | | | |
| SCT)- | 0.022899768 | 0.15983106 | 0.08214752 | 0.21467506 | −0.0015681 | 0.15272615 | −0.0154509 | 0.07184166 | 0.41092993 |
| GPR84 | | | | | | | | | |
| EFNB1)- | −0.053631236 | 0.15622061 | 0.11342297 | 0.17142263 | −0.1226944 | 0.13487951 | 0.00782776 | −0.0740351 | −0.2577697 |
| EPHB6 | | | | | | | | | |
| EFNB1)- | 0.034882273 | −0.0926104 | 0.10033884 | −0.0377687 | 0.05951038 | 0.10583498 | −0.1007403 | 0.02941392 | 0.27505478 |
| EPHB4 | | | | | | | | | |
| EFNB1)- | 0.094790674 | −0.2069445 | 0.27378386 | −0.2673604 | 0.26418627 | 0.29765131 | −0.01368 | −0.1256232 | 0.46658445 |
| ERBB2 | | | | | | | | | |
| EFNB1)- | −0.006197372 | −0.0685534 | 0.15389827 | 0.02287432 | −0.0341807 | 0.07280968 | −0.1687509 | −0.0356638 | −0.2415412 |
| EPHB1 | | | | | | | | | |
| EFNB1)- | 0.052819868 | 0.04274531 | 0.11446646 | 0.10787113 | 0.13041115 | 0.12937566 | 0.10331836 | 0.01905077 | −0.2295199 |
| EPHB3 | | | | | | | | | |
| EFNB1)- | −0.13599855 | 0.01043438 | 0.13849614 | 0.01756157 | −0.0585154 | 0.05593508 | −0.1561202 | 0.07517114 | 0.15693831 |
| EPHB2 | | | | | | | | | |
| EFNB1)- | 0.008839899 | −0.0276748 | 0.3175978 | −0.0398811 | 0.12770084 | 0.1717373 | −0.115244 | −0.0728226 | 0.00963321 |
| EPHA4 | | | | | | | | | |
| DLL3)- | 0.064369445 | 0.1056216 | 0.23746074 | 0.05552381 | 0.05414688 | 0.08935108 | −0.1119025 | 0.28160462 | 0.16622067 |
| NOTCH1 | | | | | | | | | |
| DLL3)- | 0.029778897 | 0.25086962 | 0.06705103 | 0.22872895 | −0.158618 | 0.30729848 | 0.0908136 | 0.12293634 | 0.31782737 |
| NOTCH4 | | | | | | | | | |
| DLL3)- | 0.188769688 | 0.08928072 | −0.0867188 | −0.0504337 | 0.1482506 | −0.1415965 | 0.07672962 | 0.14954754 | 0.1667875 |
| NOTCH2 | | | | | | | | | |
| DLL3)- | 0.049175142 | −0.1599709 | −0.1741618 | −0.0154741 | 0.04220478 | −0.022721 | −0.0078741 | −0.0206011 | −0.0449506 |
| NOTCH3 | | | | | | | | | |
| SEMA4G)- | −0.03362786 | −0.1038292 | −0.0410142 | −0.1244908 | −0.0271581 | −0.0019921 | −0.0826747 | −0.0767645 | 0.17173348 |
| PLXNB2 | | | | | | | | | |
| VCL)- | 0.128112659 | −0.0515415 | 0.20349753 | −0.3010441 | −0.1784106 | 0.44476409 | 0.02385067 | −0.0341861 | 0.04833401 |
| ITGB5 | | | | | | | | | |
| EFNA2)- | −0.05679843 | −0.0956751 | 0.04512241 | 0.12562436 | 0.05438661 | 0.05059094 | −0.0987197 | 0.03065814 | −0.1897967 |
| EPHA3 | | | | | | | | | |
| EFNA2)- | −0.028507444 | −0.1047539 | 0.21237316 | −0.13008 | −0.0261247 | 0.27384568 | −0.0054638 | −0.2359507 | 0.3185918 |
| EPHA1 | | | | | | | | | |
| EFNA2)- | −0.043137024 | 0.00047483 | 0.11366922 | −0.0409024 | 0.02936588 | 0.1274841 | −0.1097605 | −0.0289286 | 0.00053754 |
| EPHA4 | | | | | | | | | |
| EFNA2)- | 0.114306125 | −0.1336698 | 0.1185926 | −0.0659319 | 0.15315862 | 0.21145652 | −0.2025242 | −0.1393586 | 0.09041762 |
| EPHA2 | | | | | | | | | |
| MADCAM1)- | 0.097721883 | 0.11124642 | −0.1959394 | −0.1211719 | 0.2989812 | −0.2378155 | −0.0872086 | 0.12037438 | 0.24297417 |
| ITGA4 | | | | | | | | | |
| MADCAM1)- | −0.099137391 | 0.03075643 | −0.1930862 | −0.0013242 | 0.00313042 | −0.0333552 | −0.0336486 | −0.0151656 | −0.1351394 |
| CD44 | | | | | | | | | |
| MADCAM1)- | 0.112127424 | 0.15769516 | 0.07936134 | 0.03717988 | −0.0491261 | 0.07670064 | 0.11456781 | −0.0578525 | 0.26432743 |
| ITGB7 | | | | | | | | | |
| MIF)- | 0.062509246 | 0.01491984 | −0.053459 | 0.20754369 | −0.2067507 | 0.22519445 | −0.3064073 | 0.19630405 | 0.27067713 |
| CXCR4 | | | | | | | | | |
| MIF)- CD44 | −0.081797183 | 0.08839413 | 0.1933917 | 0.15491658 | −0.0330398 | 0.06789778 | −0.088628 | 0.20302163 | 0.35402279 |
| MIF)- | −0.078911555 | 0.08047851 | −0.0327891 | −0.0134851 | −0.0709043 | −0.1621067 | 0.07495895 | 0.17464115 | −0.1812648 |
| EGFR | | | | | | | | | |
| MIF)- | −0.099730466 | −0.0399479 | −0.0065654 | 0.04629341 | 0.02633454 | −0.0667135 | 0.00446763 | −0.0046229 | −0.1259971 |
| CXCR2 | | | | | | | | | |
| OSM)- | −0.049013591 | 0.06959427 | 0.1635579 | 0.049726 | 0.01689327 | 0.0524601 | 0.01316356 | 0.07409382 | 0.3092088 |
| LIFR | | | | | | | | | |
| OSM)- | 0.033738063 | 0.1427638 | 0.01029136 | 0.04065854 | −0.2024075 | −0.0633481 | −0.0664205 | 0.02155957 | 0.15081677 |
| IL6ST | | | | | | | | | |
| OSM)- | −0.037482464 | 0.10568586 | 0.13734565 | −0.0573363 | −0.0864361 | −0.0565106 | 0.04857213 | −0.0645114 | −0.0736029 |
| OSMR | | | | | | | | | |
| LGALS1)- | −0.276469253 | 0.33489515 | 0.26009444 | −0.2494903 | −0.059085 | 0.75951097 | 0.35966207 | −0.4289391 | 0.47471772 |
| ITGB1 | | | | | | | | | |
| LGALS1)- | 0.140106921 | 0.10070729 | −0.119756 | −0.3928647 | 0.26441665 | −0.2857283 | −0.4393954 | 0.35202343 | 0.40455159 |
| PTPRC | | | | | | | | | |
| NID2)- | −0.039071453 | 0.01919924 | 0.08748646 | 0.01856515 | 0.02227959 | 0.12800231 | 0.17219024 | 0.04443461 | 0.09248467 |
| COL13A1 | | | | | | | | | |
| GZMB)- | 0.05837056 | 0.08734223 | 0.17876662 | 0.1759681 | 0.16999423 | 0.21120235 | 0.32745574 | 0.07285857 | 0.35637056 |
| CHRM3 | | | | | | | | | |
| GZMB)- | −0.043257564 | −0.2024039 | 0.16623115 | −0.0447004 | −0.1506411 | −0.0342009 | −0.2550952 | −0.1751305 | −0.0303187 |
| IGF2R | | | | | | | | | |
| OXT)- | −0.049245142 | 0.27045332 | 0.17133702 | 0.28655157 | 0.06019148 | 0.27519362 | 0.23186194 | 0.14810659 | 0.48740535 |
| AVPR1B | | | | | | | | | |
| OXT)- | −0.057438968 | 0.23926649 | 0.22141173 | 0.19359323 | 0.17522962 | 0.27452934 | 0.17281079 | 0.00503665 | 0.50146247 |
| OXTR | | | | | | | | | |
| F9)- LRP1 | −0.035375382 | −0.1529129 | −0.1666003 | −0.1385515 | −0.1075285 | −0.2612255 | −0.3007688 | −0.3187206 | −0.3964766 |
| PCSK1N)- | −0.040358837 | −0.0904077 | −0.0269092 | 0.07525256 | −0.0654638 | −0.0793754 | −0.0191104 | −0.121583 | 0.11957267 |
| GPR171 | | | | | | | | | |
| TIMP1)- | −0.039983652 | −0.1018439 | −0.1873173 | 0.08054204 | 0.11624211 | −0.1121895 | −0.1084992 | −0.0007354 | 0.04592011 |
| FGFR2 | | | | | | | | | |
| TIMP1)- | −0.139922578 | 0.15033277 | 0.08602015 | −0.0325185 | −0.0462838 | 0.2270475 | −0.0071388 | −0.1099057 | 0.01951514 |
| CD63 | | | | | | | | | |
| MMP2)- | 0.104106218 | −0.0404804 | 0.03487657 | −0.2372963 | 0.18026235 | −0.2051959 | −0.2542283 | 0.35992542 | 0.26638076 |
| PECAM1 | | | | | | | | | |
| MMP2)- | −0.04506026 | 0.09138045 | 0.16869993 | −0.1594269 | 0.03235634 | 0.17966547 | 0.10040001 | −0.1369425 | 0.30238212 |
| SDC2 | | | | | | | | | |
| MMP2)- | −0.237340003 | 0.26818601 | 0.47913688 | −0.3379623 | −0.2702898 | 0.50751416 | 0.29865629 | −0.2936056 | 0.35180563 |
| FGFR1 | | | | | | | | | |
| CCL22)- | −0.117603537 | 0.07580203 | 0.22517241 | 0.09971831 | −0.0416351 | 0.2654345 | −0.0288413 | 0.13586121 | 0.35370142 |
| CCR4 | | | | | | | | | |
| CCL22)- | 0.126840036 | 0.05740411 | 0.23602908 | 0.0434182 | −0.132237 | 0.14780547 | 0.10831224 | 0.14631417 | 0.38356883 |
| DPP4 | | | | | | | | | |
| CCL17)- | −0.075388498 | −0.0364294 | 0.35938311 | 0.30935949 | −0.155954 | 0.26544222 | 0.00982138 | 0.14687116 | 0.48393773 |
| CCR4 | | | | | | | | | |
| TG)- | −0.042697071 | 0.15909914 | 0.23505278 | 0.11418009 | 0.08754883 | 0.34443261 | 0.1765562 | 0.23015681 | 0.62777097 |
| ASGR1 | | | | | | | | | |
| SFRP1)- | −0.011887735 | 0.0601005 | 0.10187028 | −0.0906904 | 0.0304074 | 0.09342053 | 0.25705961 | 0.05220038 | 0.07737239 |
| FZD6 | | | | | | | | | |
| PLAT)- | −0.057756784 | 0.04709198 | −0.0001511 | −0.0225883 | 0.14196003 | −0.1925094 | −0.0036963 | −0.0299271 | −0.1478609 |
| ITGAM | | | | | | | | | |
| PLAT)- | −0.054306384 | 0.17174343 | 0.13531327 | −0.1455095 | −0.162506 | 0.4187654 | 0.10704357 | −0.1150193 | 0.12914792 |
| LRP1 | | | | | | | | | |
| PLAT)- | 0.284027829 | 0.20930617 | −0.1509711 | −0.2795037 | 0.11983288 | −0.2203077 | −0.0010313 | 0.21044275 | 0.23416995 |
| ITGB2 | | | | | | | | | |
| DKK4)- | 0.130443193 | 0.05738296 | 0.17773776 | 0.03822464 | 0.0293382 | 0.09161395 | 0.15091722 | −0.0898667 | 0.26391582 |
| LRP6 | | | | | | | | | |
| DKK4)- | 0.023391043 | −0.0667309 | 0.03967429 | −0.1359669 | −0.1226408 | 0.1071584 | 0.06227315 | 0.04496554 | −0.2477627 |
| LRP5 | | | | | | | | | |
| LHB)- | −0.059594652 | 0.06215093 | 0.13450547 | 0.1759268 | −0.0608091 | 0.33044375 | 0.19664028 | 0.15018253 | 0.37325412 |
| PTH1R | | | | | | | | | |
| LHB)- | −0.165815217 | 0.18445666 | 0.22266862 | 0.23984576 | −0.2124871 | 0.32974252 | −0.0495398 | 0.21105112 | 0.52177798 |
| RAMP2 | | | | | | | | | |
| LHB)- | −0.081333773 | −0.0451219 | 0.15835305 | 0.17074711 | −0.0377477 | 0.12456956 | −0.1270334 | 0.294113 | 0.15216134 |
| VIPR1 | | | | | | | | | |
| LHB)- | −0.056867835 | 0.26267053 | 0.253625 | 0.11900646 | −0.1304175 | 0.26053973 | 0.20672226 | 0.07681197 | 0.37535738 |
| ADRB2 | | | | | | | | | |
| LHB)- | −0.111829977 | 0.16933561 | 0.25752627 | 0.31433939 | 0.10344843 | 0.21680733 | 0.31585241 | 0.0124857 | 0.47514764 |
| ADRB3 | | | | | | | | | |
| LHB)- | −0.059065673 | 0.27682867 | 0.11182208 | 0.19883763 | 0.10757305 | 0.25616877 | 0.3447013 | 0.00199527 | 0.51592748 |
| LHCGR | | | | | | | | | |
| LHB)- | −0.063804022 | 0.20218292 | 0.27976089 | 0.21781306 | −0.0764148 | 0.19448604 | 0.16653849 | 0.08890093 | 0.30358945 |
| GPR84 | | | | | | | | | |
| AMH)- | −0.030277165 | −0.0090837 | 0.05765385 | −0.0842642 | −0.0441779 | 0.18012192 | −0.0603761 | 0.03346531 | 0.00375834 |
| ACVR1 | | | | | | | | | |
| AMH)- | −0.0442225 | 0.06388258 | −0.0377164 | −0.0743356 | 0.02433024 | 0.01487293 | 0.12318836 | −0.0566373 | −0.0696284 |
| EGFR | | | | | | | | | |
| EBI3)- | −0.102030125 | 0.17436621 | 0.19165992 | 0.30010532 | 0.1475106 | 0.18652101 | 0.14844086 | 0.15781101 | 0.31916174 |
| IL27RA | | | | | | | | | |
| EBI3)- | 0.07843349 | −0.012635 | 0.22625005 | 0.1134486 | −0.027678 | 0.08242267 | −0.0338773 | 0.03353371 | 0.34675548 |
| IL6ST | | | | | | | | | |
| TGFB1)- | 0.010803597 | −0.1376593 | 0.2907662 | −0.1556376 | −0.1572915 | 0.18988306 | −0.1817327 | −0.0563356 | 0.12069407 |
| ENG | | | | | | | | | |
| TGFB1)- | −0.037106053 | 0.12962399 | −0.1647137 | −0.0374423 | 0.21081105 | 0.07387218 | 0.1420067 | 0.01209676 | −0.0345695 |
| EGFR | | | | | | | | | |
| TGFB1)- | −0.072324912 | −0.0558997 | −0.1041684 | 0.088144 | −0.0921668 | −0.0609892 | 0.24942657 | 0.19618366 | −0.302567 |
| SMAD3 | | | | | | | | | |
| TGFB1)- | −0.080999094 | 0.12633642 | 0.04436469 | 0.04083695 | −0.0362926 | 0.03494587 | −0.2661185 | −0.0653416 | 0.02266709 |
| TGFBR2 | | | | | | | | | |
| TGFB1)- | −0.053342482 | −0.0042713 | −0.0181968 | −0.0796531 | −0.1363086 | −0.0239802 | −0.0366403 | 0.00634201 | 0.11770392 |
| ITGB3 | | | | | | | | | |
| TGFB1)- | −0.080779265 | −0.2734533 | 0.15783991 | −0.0253123 | −0.0523602 | 0.34353943 | −0.1018544 | −0.0644931 | 0.12671714 |
| ACVRL1 | | | | | | | | | |
| TGFB1)- | −0.20966719 | 0.1694302 | −0.1875678 | 0.13566442 | 0.00458466 | −0.0020829 | 0.25811573 | 0.18461755 | 0.10986143 |
| ITGB6 | | | | | | | | | |
| TGFB1)- | 0.139979931 | 0.16623676 | 0.20163823 | −0.0374124 | 0.05511189 | 0.21934056 | 0.15100353 | −0.0456448 | 0.08413597 |
| ITGAV | | | | | | | | | |
| TGFB1)- | 0.231591314 | −0.03127 | 0.06406904 | −0.0037175 | −0.0331119 | 0.11575227 | 0.11853206 | −0.3231602 | 0.32863811 |
| ITGB1 | | | | | | | | | |
| TGFB1)- | 0.045480114 | −0.1724122 | 0.07594538 | 0.09729682 | 0.08113263 | −0.0762981 | 0.24299066 | −0.3347117 | 0.54106323 |
| CAV1 | | | | | | | | | |
| TGFB1)- | −0.015884944 | −0.1266762 | 0.26397657 | −0.0469098 | −0.1385427 | 0.24039621 | 0.12706945 | −0.1346047 | 0.29662308 |
| SDC2 | | | | | | | | | |
| TGFB1)- | −0.085840607 | −0.007214 | −0.1586865 | 0.1873636 | −0.0256494 | 0.01840094 | 0.08321896 | 0.18730417 | −0.1127504 |
| ITGB8 | | | | | | | | | |
| TGFB1)- | −0.232930669 | −0.3030258 | 0.31356059 | −0.1241894 | 0.10352975 | −0.0967021 | −0.1888087 | 0.16412987 | 0.21603745 |
| CXCR4 | | | | | | | | | |
| TGFB1)- | 0.221423089 | −0.023848 | 0.12126721 | 0.11804797 | −0.0348404 | 0.10102271 | 0.03119736 | −0.3071148 | 0.23154269 |
| ITGB5 | | | | | | | | | |
| ICAM5)- | 0.008268194 | 0.15168716 | −0.0115058 | 0.07725892 | 0.05635675 | 0.13946477 | −0.1036661 | 0.09869845 | 0.36674332 |
| ITGAL | | | | | | | | | |
| ICAM5)- | 0.020382536 | 0.12183783 | −0.1647095 | −0.0074836 | −0.0098355 | −0.0123168 | −0.1229699 | 0.22614866 | 0.28553894 |
| ITGB2 | | | | | | | | | |
| CEACAM5)- | 0.121223758 | 0.20232589 | 0.04524383 | 0.14149856 | −0.0239382 | 0.08651464 | −0.0267435 | −0.1636525 | −0.1362666 |
| CD1D | | | | | | | | | |
| COMP)- | −0.351362079 | 0.16948249 | 0.20323398 | −0.3755145 | −0.1673632 | 0.44312729 | 0.13588536 | −0.4326271 | 0.11516058 |
| ITGB1 | | | | | | | | | |
| COMP)- | 0.226447495 | −0.0242169 | −0.0617689 | 0.02951947 | 0.15860023 | −0.1999285 | 0.00840059 | 0.14746629 | 0.22542172 |
| ITGB3 | | | | | | | | | |
| COMP)- | 0.11858751 | 0.09165021 | 0.04266127 | 0.07771732 | 0.02109388 | −0.1679248 | 0.09595645 | 0.13742833 | 0.30264079 |
| CD36 | | | | | | | | | |
| HGF)- | −0.003072645 | −0.1915496 | 0.10278879 | −0.1326979 | −0.0748475 | −0.1756634 | −0.1638571 | −0.0241963 | −0.0879764 |
| CD44 | | | | | | | | | |
| HGF)- | 0.053740392 | −0.1999232 | 0.04430693 | 0.1131997 | −0.2107341 | −0.1235664 | −0.0954302 | −0.16495 | −0.0679695 |
| SDC1 | | | | | | | | | |
| HGF)- | 0.069718923 | −0.2392498 | −0.1205757 | −0.0122751 | −0.0780893 | −0.132752 | −0.1193144 | −0.2976899 | −0.1292157 |
| ITGB1 | | | | | | | | | |
| HGF)- MET | −0.157102659 | −0.2099573 | −0.0057702 | 0.01640202 | 0.09440041 | 0.08980982 | 0.10584825 | 0.01435966 | −0.0674452 |
| HGF)- | 0.064582073 | −0.0373376 | −0.031567 | 0.08946928 | −0.0154956 | 0.03663555 | −0.0321534 | −0.1700959 | 0.26423396 |
| SDC2 | | | | | | | | | |
| HGF)- | −0.022771634 | −0.0645167 | 0.00124706 | 0.18505394 | 0.01759624 | −0.1016753 | −0.1266069 | −0.0958133 | 0.19275019 |
| NRP1 | | | | | | | | | |
| HGF)- ST14 | −0.079739541 | −0.0587377 | −0.0234968 | −0.2514276 | 0.13564547 | 0.13990645 | 0.21801213 | 0.09441565 | −0.3280996 |
| LAMB1)- | 0.05278147 | −0.1519513 | 0.04781227 | −0.0142076 | −0.113579 | −0.0874942 | 0.06057525 | −0.0547016 | −0.0911988 |
| ITGA7 | | | | | | | | | |
| LAMB1)- | −0.340425751 | 0.2253104 | 0.38583987 | 0.00784493 | −0.0808149 | 0.68072531 | 0.39284412 | −0.3148354 | 0.33239595 |
| ITGAV | | | | | | | | | |
| LAMB1)- | −0.186498521 | 0.21206129 | 0.24409042 | −0.133364 | −0.0957386 | 0.46586557 | 0.24084294 | −0.3032054 | 0.34917561 |
| ITGB1 | | | | | | | | | |
| LAMB1)- | 0.255743343 | 0.00299379 | 0.20799213 | 0.05414415 | −0.0531041 | −0.1367619 | 0.25926251 | 0.30105807 | −0.1241712 |
| ITGA6 | | | | | | | | | |
| LAMB1)- | −0.169556764 | 0.06612708 | 0.1377495 | −0.1442101 | −0.0804157 | 0.17192905 | 0.17332154 | −0.3249058 | 0.1566369 |
| ITGA1 | | | | | | | | | |
| LAMB1)- | 0.104577672 | −0.023508 | 0.13689152 | 0.4174083 | −0.0128044 | −0.0184528 | 0.08591432 | 0.06582964 | 0.15826877 |
| ITGA2 | | | | | | | | | |
| NAMPT)- | −0.16823107 | −0.0612827 | −0.1617217 | 0.09927221 | 0.0754222 | −0.153176 | 0.07654851 | 0.08555304 | 0.15492617 |
| ADORA2A | | | | | | | | | |
| PON2)- | 0.175716627 | 0.01481957 | 0.17258274 | 0.0111725 | 0.18660939 | 0.24028743 | −0.2873867 | −0.1565197 | 0.05621384 |
| HTR2A | | | | | | | | | |
| PDAP1)- | −0.090968872 | −0.0123117 | −0.1868612 | 1.76E−05 | 0.03949162 | 0.05591568 | −0.0840495 | −0.0372603 | 0.01538193 |
| PDGFRB | | | | | | | | | |
| C5)- | −0.048883502 | −0.0154174 | 0.11102644 | −0.081231 | −0.0053921 | 0.1094852 | 0.17323615 | 0.23088021 | −0.0031461 |
| ADRA2A | | | | | | | | | |
| C5)- C5AR2 | 0.082258619 | 0.16725382 | 0.21629609 | 0.21269552 | 0.15345179 | 0.0623978 | 0.03011095 | 0.18173828 | 0.45998163 |
| C5)- C5AR1 | 0.009346535 | 0.05475562 | −0.0438406 | 0.14531172 | 0.0790626 | 0.1151253 | −0.038902 | 0.16228987 | 0.30890032 |
| TNFSF8)- | 0.222739899 | 0.15426045 | −0.0626762 | 0.13997132 | 0.04387952 | 0.18753653 | 0.15175365 | 0.19274087 | 0.3078719 |
| TNFRSF8 | | | | | | | | | |
| CSF3)- | −0.006866788 | 0.12672041 | −0.0182322 | 0.25878028 | −0.1153337 | 0.1721171 | 0.00184346 | −0.0053026 | 0.27253234 |
| CSF1R | | | | | | | | | |
| CSF3)- | 0.093158797 | 0.19303578 | −0.083769 | 0.15610565 | 0.02121735 | 0.07694202 | −0.1610094 | 0.24331383 | 0.41093691 |
| CSF3R | | | | | | | | | |
| WNT3)- | 0.028436156 | 0.07675296 | 0.02960823 | −0.0917059 | 0.04823293 | 0.08486917 | 0.03234148 | 0.0692007 | −0.128702 |
| RYK | | | | | | | | | |
| WNT3)- | 0.092242636 | 0.14604364 | 0.07998266 | 0.09330152 | 0.13786081 | −0.0026681 | 0.01213197 | −0.0809714 | 0.29102978 |
| LRP6 | | | | | | | | | |
| WNT3)- | 0.002563839 | 0.15284725 | 0.15205847 | 0.21636481 | 0.08525414 | 0.1997435 | 0.26609193 | −0.1359348 | 0.47740824 |
| ROR2 | | | | | | | | | |
| WNT3)- | −0.016646487 | 0.17786961 | 0.26870431 | 0.23816046 | 0.14148779 | 0.06525902 | 0.06397567 | −0.0900687 | 0.12020505 |
| FZD1 | | | | | | | | | |
| WNT3)- | 0.096867581 | 0.1117427 | 0.22140325 | 0.13057662 | 0.21356696 | 0.08570709 | 0.17458146 | 0.00040302 | 0.37681142 |
| FZD7 | | | | | | | | | |
| WNT3)- | −0.085914793 | 0.06645093 | 0.26693488 | 0.12400535 | −0.015629 | 0.26434853 | 0.17408935 | 0.15216169 | 0.32975808 |
| FZD8 | | | | | | | | | |
| WNT3)- | 0.048207246 | −0.16333 | 0.09035786 | −0.2329924 | 0.17521696 | 0.14677562 | 0.2333576 | 0.11470747 | −0.0386679 |
| FZD5 | | | | | | | | | |
| CCL2)- | 0.153067848 | 0.04528608 | −0.0991855 | 0.10731785 | 0.02328107 | 0.05058362 | 0.12369963 | 0.09700285 | 0.36601684 |
| CCR4 | | | | | | | | | |
| CCL2)- | −0.048755472 | −0.1449041 | 0.246784 | 0.06000861 | −0.0656111 | 0.03168066 | 0.06768815 | 0.00412222 | 0.27112373 |
| CCR1 | | | | | | | | | |
| CCL2)- | −0.076570399 | 0.04639525 | −0.020164 | 0.0678338 | 0.06845279 | 0.12484562 | 0.18431745 | 0.27420877 | 0.35964713 |
| CCR5 | | | | | | | | | |
| CCL2)- | −0.053812704 | 0.10528305 | 0.05726301 | 0.06404551 | 0.03145264 | 0.28115684 | 0.22722565 | 0.2696069 | 0.39684746 |
| CCR3 | | | | | | | | | |
| CCL13)- | 0.080162345 | 0.29810587 | 0.27254551 | 0.15968465 | 0.04174322 | 0.18997326 | 0.2393642 | 0.26622858 | 0.50086122 |
| CCR3 | | | | | | | | | |
| CCL13)- | −0.224887711 | −0.0829349 | 0.2250683 | 0.18262732 | −0.0759964 | 0.30353865 | −0.051793 | 0.03400899 | 0.38855104 |
| CCR1 | | | | | | | | | |
| CCL13)- | 0.068406396 | −0.043215 | 0.19601914 | 0.22301488 | 0.15293533 | 0.20478498 | 0.17859904 | 0.35073286 | 0.59616139 |
| CCR5 | | | | | | | | | |
| COL1A1)- | −0.322499 | 0.2980936 | 0.33580722 | −0.2349667 | −0.1650137 | 0.48195425 | 0.26390482 | −0.2520297 | 0.35403778 |
| ITGA5 | | | | | | | | | |
| COL1A1)- | 0.102526847 | 0.07158675 | −0.0946206 | −0.2382944 | 0.1342957 | −0.1485702 | −0.0425264 | −0.0001113 | 0.04811703 |
| CD93 | | | | | | | | | |
| COL1A1)- | 0.202792789 | −0.0445785 | −0.0722436 | −0.1755076 | 0.07103895 | −0.2194124 | −0.1067372 | 0.18671807 | 0.00248407 |
| FLT4 | | | | | | | | | |
| COL1A1)- | 0.225323519 | −0.0840278 | −0.0691996 | −0.0479736 | −0.0191467 | −0.337436 | −0.1230473 | 0.11776976 | −0.0326972 |
| CD36 | | | | | | | | | |
| COL1A1)- | −0.341121104 | 0.28367589 | 0.17030707 | −0.2128858 | −0.0959766 | 0.12385012 | 0.22282147 | −0.3519977 | 0.35218989 |
| ITGA1 | | | | | | | | | |
| COL1A1)- | −0.082395982 | 0.0618204 | 0.05977005 | −0.2515643 | −0.1573343 | 0.44237797 | 0.07741047 | −0.0843772 | 0.14896771 |
| DDR2 | | | | | | | | | |
| COL1A1)- | 0.127042109 | 0.03465387 | −0.1909967 | −0.0131644 | 0.27456483 | 0.01603222 | −0.2626449 | 0.17122226 | 0.13397544 |
| CD44 | | | | | | | | | |
| COL1A1)- | −0.34933389 | 0.08672549 | 0.25505208 | −0.1090411 | −0.0548086 | 0.757887 | 0.24307678 | −0.2884902 | 0.1355397 |
| ITGAV | | | | | | | | | |
| COL1A1)- | −0.407666113 | 0.43743034 | 0.44219863 | −0.268461 | −0.1406074 | 0.68699734 | 0.41461028 | −0.334986 | 0.44987997 |
| ITGB1 | | | | | | | | | |
| COL1A1)- | −0.3258883 | 0.35984955 | 0.39946041 | −0.4856112 | −0.3749709 | 0.66593126 | 0.34429747 | −0.2327916 | 0.39381796 |
| ITGA11 | | | | | | | | | |
| COL1A1)- | −0.016674185 | −0.2218235 | 0.04459612 | 0.32217649 | −0.0348209 | −0.0181447 | −0.0082607 | −0.0044293 | −0.1014778 |
| ITGA2 | | | | | | | | | |
| COL1A1)- | 0.266708849 | −0.0597643 | 0.28042979 | 0.1851017 | −0.2788597 | −0.2928911 | 0.3138885 | 0.35666245 | −0.0229748 |
| DDR1 | | | | | | | | | |
| VTN)- | −0.087084116 | −0.0521229 | −0.0720935 | 0.10470621 | −0.1225194 | 0.07539365 | −0.2138066 | 0.08846771 | 0.11435518 |
| ITGA5 | | | | | | | | | |
| VTN)- | −0.058891088 | 0.15187442 | 0.15140379 | 0.27558597 | 0.12951912 | 0.07833074 | 0.06726683 | 0.10302121 | 0.40679783 |
| ITGA8 | | | | | | | | | |
| VTN)- | 0.094101008 | −0.0866809 | 0.04631149 | 0.04945442 | −0.114228 | −0.035067 | −0.0850237 | −0.0018983 | −0.1124091 |
| CD47 | | | | | | | | | |
| VTN)- | −0.109271725 | 0.16978075 | 0.19750855 | −0.098718 | −0.1723877 | 0.05499791 | −0.0919446 | −0.0546503 | 0.02296009 |
| ITGAV | | | | | | | | | |
| VTN)- | −0.08213201 | −0.1558794 | −0.1035598 | −0.0806201 | −0.1615681 | −0.187562 | −0.1682138 | −0.1915303 | −0.1856319 |
| ITGB1 | | | | | | | | | |
| VTN)- KDR | 0.075055929 | 0.21602085 | 0.07305617 | 0.14334598 | 0.00221421 | 0.15132667 | −0.1019608 | 0.28538843 | 0.46209582 |
| VTN)- | −0.144261078 | 0.01550259 | −0.1468461 | −0.0130165 | 0.01402804 | 0.08362268 | 0.00930408 | 0.02669866 | 0.06968583 |
| PLAUR | | | | | | | | | |
| VTN)- | −0.014203819 | 0.20109968 | 0.37770462 | 0.21885181 | −0.040111 | 0.27317859 | 0.16493514 | 0.26937172 | 0.38091542 |
| ITGA2B | | | | | | | | | |
| VTN)- | 0.00125117 | 0.20062026 | 0.11590845 | 0.16934025 | −0.0748333 | 0.1446859 | 0.13956851 | 0.091763 | 0.27416385 |
| ITGB3 | | | | | | | | | |
| VTN)- | −0.036139473 | 0.07342262 | −0.028686 | −0.2643777 | 0.09431232 | 0.09379028 | 0.07852408 | 0.13471988 | −0.2609334 |
| ITGB6 | | | | | | | | | |
| VTN)- | 0.042036931 | −0.1035489 | 0.24479344 | 0.09695224 | 0.10697763 | 0.21911791 | 0.04982131 | 0.12146337 | 0.31149743 |
| TNFRSF11B | | | | | | | | | |
| VTN)- PVR | −0.04511806 | 0.09693003 | 0.24602043 | −0.0816468 | −0.0742093 | 0.13424313 | −0.0754771 | −0.0277784 | 0.0846342 |
| VTN)- | −0.042372938 | −0.1334392 | −0.016762 | −0.1004144 | −0.2694245 | −0.1781324 | −0.2040636 | −0.1781941 | −0.1843899 |
| ITGB5 | | | | | | | | | |
| BSP)- | 0.059961174 | 0.28553575 | 0.16359222 | 0.09107823 | −0.0443783 | 0.09407625 | 0.08922659 | 0.01743327 | 0.34452445 |
| ITGB3 | | | | | | | | | |
| TBSP)- | 0.010516212 | −0.0663261 | −0.0796966 | −0.0850741 | −0.146351 | −0.059778 | −0.0996315 | −0.0391561 | −0.0168935 |
| ITGAV | | | | | | | | | |
| CXCL6)- | −0.002086364 | −0.1823392 | 0.16687963 | 0.08332369 | −0.0557592 | 0.15846122 | −0.036704 | 0.09411878 | −0.0352693 |
| ADRA2A | | | | | | | | | |
| CXCL6)- | 0.235673014 | 0.22185302 | −0.0653586 | 0.16676725 | 0.07858174 | 0.05331659 | 0.11429069 | 0.13629825 | 0.25458295 |
| CXCR2 | | | | | | | | | |
| CXCL6)- | −0.020298477 | 0.10862106 | 0.16255554 | −0.0166806 | −0.200408 | 0.16005625 | 0.07426959 | 0.02743928 | 0.24514313 |
| CXCR1 | | | | | | | | | |
| IL2)- | 0.111269616 | 0.04631598 | 0.08655812 | 0.07157411 | 0.01073068 | 0.16312175 | 0.15227283 | 0.22906911 | 0.46017564 |
| IL2RA | | | | | | | | | |
| IL2)- | 0.109825791 | 0.13175334 | 0.08309763 | −0.0883668 | 0.02161926 | 0.09882744 | −0.0927633 | 0.31206407 | 0.39374699 |
| IL2RG | | | | | | | | | |
| IL2)- CD53 | −0.026888419 | −0.0037993 | 0.10437298 | −0.0320753 | 0.05338944 | −0.0650999 | −0.1555917 | 0.24179346 | 0.40168554 |
| APOC3)- | −0.043679309 | −0.0814864 | 0.0313119 | −0.169569 | 0.09462447 | 0.11444086 | 0.13754143 | 0.09256137 | −0.3151362 |
| LDLR | | | | | | | | | |
| APOC3)- | −0.046680608 | −0.2993666 | −0.1956136 | −0.1980934 | −0.0933824 | −0.1741821 | −0.1996877 | −0.2635576 | −0.2650055 |
| LRP1 | | | | | | | | | |
| APOC3)- | −0.018441594 | 0.01235127 | −0.0731182 | 0.08583382 | −0.0916904 | 0.01869105 | −0.0562613 | −0.1532812 | 0.30950512 |
| SDC2 | | | | | | | | | |
| APOC3)- | −0.076326744 | −0.0268915 | 0.14304095 | 0.16971379 | −0.1283596 | 0.23133479 | −0.1527847 | 0.13117102 | 0.27631319 |
| TLR2 | | | | | | | | | |
| KITLG)- | 0.023878888 | 0.24082127 | 0.08264755 | 0.11281919 | 0.06052273 | 0.03215788 | 0.18310902 | 0.14994609 | 0.27360808 |
| KIT | | | | | | | | | |
| SELPLG)- | 0.030926161 | 0.05752606 | 0.21502708 | 0.13013906 | −0.0753979 | 0.13726771 | 0.11217302 | 0.00602293 | 0.28143913 |
| ITGAM | | | | | | | | | |
| SELPLG)- | 0.043897238 | 0.0406003 | 0.04996386 | 0.13266417 | 0.08869142 | 0.18703624 | −0.0319696 | 0.17008103 | 0.34093299 |
| ESAM | | | | | | | | | |
| SELPLG)- | −0.13943355 | 0.00583187 | 0.17866667 | 0.18086693 | 0.08927347 | 0.18763893 | 0.13422879 | 0.00406842 | 0.23210433 |
| SELL | | | | | | | | | |
| SELPLG)- | 0.104261627 | 0.1089239 | 0.17265274 | 0.16592542 | 0.06368308 | 0.15119303 | 0.18111246 | 0.12864665 | 0.37079654 |
| SELP | | | | | | | | | |
| SELPLG)- | −0.0256293 | 0.00316357 | 0.0780514 | 0.33318929 | 0.08861105 | 0.12757877 | 0.15384582 | 0.03566583 | 0.37042364 |
| SELE | | | | | | | | | |
| SELPLG)- | 0.05025372 | 0.14949493 | −0.0404458 | 0.02409173 | −0.0707249 | −0.0618965 | −0.2806524 | 0.21759094 | 0.30793702 |
| ITGB2 | | | | | | | | | |
| IL23A)- | −0.096414854 | −0.0452755 | 0.09432964 | 0.16121165 | −0.0308924 | 0.27393487 | 0.15554811 | 0.2165085 | 0.37134182 |
| IL12RB2 | | | | | | | | | |
| IFNG)- | −0.127334791 | −0.0745928 | 0.00167008 | −0.2061791 | 0.12893217 | 0.03563931 | −0.0196416 | −0.0443503 | −0.2217506 |
| IFNGR2 | | | | | | | | | |
| IFNG)- | 0.013506485 | 0.00053324 | −0.0853435 | −0.1688463 | 0.02156093 | −0.0595106 | −0.2057569 | 0.10129823 | −0.118178 |
| IFNGR1 | | | | | | | | | |
| GNB3)- | 0.003404047 | 0.0884474 | 0.01903213 | 0.01210299 | −0.0914461 | 0.08244224 | 0.0859291 | 0.17709385 | 0.45087405 |
| GABBR2 | | | | | | | | | |
| ULBP1)- | −0.054714357 | 0.05276594 | 0.15561138 | 0.04877756 | 0.11248209 | 0.02567108 | 0.2968456 | 0.07842276 | 0.25801207 |
| KLRK1 | | | | | | | | | |
| LAMA4)- | 0.069422573 | 0.01613466 | 0.03739942 | 0.0679405 | −0.0765819 | −0.206563 | 0.06719287 | 0.18793967 | −0.1438916 |
| ITGA6 | | | | | | | | | |
| LAMA4)- | −0.059538373 | −0.0389903 | 0.24890642 | 0.01483865 | 0.01280492 | 0.36945403 | 0.21785224 | −0.1026505 | 0.21439441 |
| ITGAV | | | | | | | | | |
| LAMA4)- | −0.071532792 | 0.08286187 | 0.18167063 | −0.1507827 | −0.0479567 | 0.38870315 | 0.18008029 | −0.3379305 | 0.31845917 |
| ITGB1 | | | | | | | | | |
| HBEGF)- | −0.060514532 | 0.02954528 | −0.0641951 | 0.10074054 | −0.2933343 | 0.04699286 | −0.076892 | −0.0353336 | −0.2944495 |
| CD44 | | | | | | | | | |
| HBEGF)- | −0.15997281 | 0.26664714 | 0.212058 | 0.10370379 | 0.03669594 | −0.0972499 | 0.15039433 | −0.0355754 | −0.0006825 |
| EGFR | | | | | | | | | |
| HBEGF)- | −0.070503018 | 0.09135453 | −0.0139364 | −0.0302368 | −0.2285298 | −0.1430333 | −0.1091738 | −0.0904283 | −0.0533371 |
| CD9 | | | | | | | | | |
| HBEGF)- | 0.023642428 | −0.2258012 | 0.10244068 | −0.435194 | 0.26467577 | 0.27218214 | −0.0018982 | 0.10509973 | −0.4585214 |
| ERBB2 | | | | | | | | | |
| IL4)- | 0.143481917 | 0.08296888 | 0.15667683 | 0.0723709 | 0.00361011 | 0.28593179 | 0.22725564 | 0.03128388 | 0.4709594 |
| IL13RA2 | | | | | | | | | |
| IL4)- | 0.094191717 | 0.23391216 | −0.0247596 | −0.0271149 | 0.08623762 | 0.07259747 | 0.06073314 | 0.17907497 | 0.32482603 |
| IL2RG | | | | | | | | | |
| IL4)- CD53 | 0.00111395 | 0.10153068 | 0.07786578 | 0.01373022 | −0.0414268 | 0.02279287 | −0.0986875 | 0.06992876 | 0.2663046 |
| IL4)- | −0.135697745 | −0.0387597 | 0.03391782 | 0.0156714 | 0.02377673 | 0.0957012 | 0.08898928 | −0.2053221 | 0.14984965 |
| IL13RA1 | | | | | | | | | |
| IL4)- IL4R | −0.178940143 | −0.1007347 | 0.10857381 | 0.04992873 | −0.044702 | −0.0402329 | −0.1146005 | −0.0396969 | −0.1486914 |
| SEMA3G)- | 0.084791237 | 0.03726381 | 0.13100388 | −0.0024994 | −0.1006286 | −0.0604796 | −0.1139376 | −0.0232271 | 0.06191441 |
| NRP2 | | | | | | | | | |
| LTF)- LRP1 | −0.079176197 | −0.1698883 | −0.1499114 | −0.2161238 | −0.0997144 | −0.0654938 | −0.0952296 | −0.3049361 | −0.2167288 |
| LTF)- | −0.057308575 | −0.0712998 | 0.02452903 | 0.0307592 | −0.0623788 | 0.18025112 | −0.113377 | 0.10040853 | −0.227729 |
| TFRC | | | | | | | | | |
| HRG)- | 0.002578755 | −0.2285171 | 0.0470237 | −0.2313286 | 0.21371302 | 0.16923528 | 0.31447512 | 0.35585299 | −0.6098973 |
| ERBB2 | | | | | | | | | |
| HRG)- | 0.131587708 | 0.14718803 | 0.12358698 | 0.10941273 | 0.12139485 | −0.0062232 | 0.14743299 | 0.14329027 | 0.32252305 |
| FCGR1A | | | | | | | | | |
| HRG)- | 0.233714642 | −0.0833763 | 0.30704066 | −0.1131909 | −0.0167739 | 0.00580429 | 0.2686885 | 0.33073106 | −0.3020498 |
| ERBB3 | | | | | | | | | |
| TFPI)- | −0.205987775 | −0.0246073 | −0.0854932 | −0.1231129 | −0.1473466 | −0.1272376 | 0.20759575 | 0.20705414 | 0.3386354 |
| LRP1 | | | | | | | | | |
| TFPI)- | 0.173065797 | 0.00231866 | 0.12527775 | −0.1520642 | 0.07032556 | 0.19042123 | 0.12460747 | 0.09505648 | 0.41560686 |
| SDC4 | | | | | | | | | |
| TFPI)- F3 | −0.053046505 | −0.0278446 | 0.02932166 | −0.143914 | 0.04063682 | −0.1058195 | 0.14557953 | 0.24984437 | 0.37942312 |
| APOB)- | −0.093849341 | −0.0552663 | 0.14502286 | 0.07132915 | −0.0107553 | 0.15844089 | 0.12040285 | 0.094981 | 0.18262907 |
| ITGAM | | | | | | | | | |
| APOB)- | −0.028614856 | 0.24002758 | 0.09143418 | −0.0145448 | 0.02011069 | 0.02736179 | 0.08552122 | −0.0457712 | 0.24169901 |
| LRP6 | | | | | | | | | |
| APOB)- | −0.096087599 | 0.04777501 | 0.05040702 | 0.06161046 | −0.0166052 | 0.12009542 | −0.0066544 | −0.0090463 | 0.23546471 |
| OLR1 | | | | | | | | | |
| APOB)- | −0.018471411 | 0.02667771 | 0.04788059 | −0.0633923 | −0.0397912 | −0.0128085 | 0.14044945 | 0.1311547 | −0.2281011 |
| LDLR | | | | | | | | | |
| APOB)- | −0.134524271 | 0.0182179 | 0.27747615 | 0.13568827 | 0.07455479 | 0.08397251 | 0.15860701 | 0.20593206 | 0.35982078 |
| CALCR | | | | | | | | | |
| APOB)- | 0.096635292 | 0.19614454 | 0.13937029 | 0.06529263 | −0.0888313 | 0.03170163 | 0.04650604 | 0.01527617 | 0.42896814 |
| MTTP | | | | | | | | | |
| APOB)- | 0.059829406 | 0.04799776 | −0.0336896 | −0.1695936 | −0.0363949 | 0.0267218 | 0.1677388 | 0.18450037 | −0.2410421 |
| LSR | | | | | | | | | |
| APOB)- | 0.067962662 | 0.11545311 | 0.1963122 | 0.19172941 | −0.167621 | 0.16365459 | −0.060568 | 0.05772324 | 0.43852526 |
| ADRB2 | | | | | | | | | |
| APOB)- | −0.036753056 | 0.26700384 | 0.11844665 | −0.0193476 | 0.06065932 | 0.07308863 | −0.1438301 | 0.09999209 | 0.24351484 |
| TLR6 | | | | | | | | | |
| APOB)- | 0.075568427 | −0.0885835 | −0.0509478 | −0.0389208 | 0.02224869 | −0.1708587 | −0.0228429 | −0.1895184 | −0.1175537 |
| LRP1 | | | | | | | | | |
| APOB)- | −0.079731138 | 0.00447827 | −0.0164041 | −0.0321078 | 0.0994421 | 0.06397827 | −0.1964387 | 0.27660743 | 0.32593973 |
| ITGB2 | | | | | | | | | |
| APOB)- | −0.070552969 | 0.05842426 | 0.0990699 | 0.12439049 | 0.13772808 | −0.0889071 | 0.11100463 | −0.01702 | 0.36165507 |
| TLR4 | | | | | | | | | |
| APOB)- | 0.019977542 | −0.0523987 | 0.15011433 | 0.12097717 | 0.08949872 | 0.08774347 | 0.09444265 | 0.15298802 | 0.27952375 |
| LRP8 | | | | | | | | | |
| PROC)- | −0.01652246 | 0.14057224 | 0.00055026 | 0.09216402 | 0.05609776 | 0.24701434 | −0.0683146 | 0.25954927 | 0.39739815 |
| ITGAM | | | | | | | | | |
| PROC)- | 0.15214583 | 0.17139275 | −0.096809 | 0.07316494 | −0.0089335 | 0.03059384 | −0.1528425 | 0.13711961 | 0.18411478 |
| ITGB2 | | | | | | | | | |
| PROC)- | 0.054852675 | 0.05929482 | −0.0292115 | −0.0562027 | −0.0533278 | 0.00939845 | 0.06110018 | 0.04382109 | 0.17495844 |
| THBD | | | | | | | | | |
| APOA1)- | 0.053930209 | 0.13479934 | −0.0626002 | 0.03825453 | 0.05844516 | −0.1053507 | −0.0320932 | −0.0767605 | 0.2447821 |
| ABCA1 | | | | | | | | | |
| APOA1)- | 0.127186391 | −0.1535799 | 0.19247139 | −0.0924346 | −0.0978065 | −0.0567379 | 0.10214032 | 0.1025475 | −0.1786858 |
| LDLR | | | | | | | | | |
| APOA1)- | −0.06263254 | −0.1531633 | −0.1644649 | −0.2381375 | −0.0931701 | −0.3520808 | −0.2804996 | −0.1378333 | −0.2801678 |
| LRP1 | | | | | | | | | |
| FGF23)- | −0.02560457 | 0.31492368 | 0.10789034 | −0.0744183 | −0.0515396 | 0.18577603 | 0.1620703 | 0.07487485 | 0.44819268 |
| FGFR2 | | | | | | | | | |
| FGF23)- | 0.189863112 | 0.18862859 | 0.22342319 | 0.08693652 | −0.0177917 | 0.13075417 | 0.23702413 | 0.3525696 | 0.42125167 |
| PHEX | | | | | | | | | |
| FGF23)- KL | −0.007732813 | 0.22392916 | 0.21911228 | 0.00210495 | −0.0871274 | 0.20608811 | 0.12494177 | 0.05405596 | 0.44950765 |
| FGF23)- | −0.078184468 | −0.0140111 | −0.0410116 | −0.0527321 | −0.0699928 | 0.05831062 | −0.1359078 | −0.0267683 | 0.0615573 |
| FGFR4 | | | | | | | | | |
| FGF23)- | −0.14311594 | 0.15346246 | 0.12601173 | 0.07515055 | −0.0782532 | 0.01466463 | −0.008337 | −0.1185444 | 0.31274606 |
| FGFR1 | | | | | | | | | |
| FGF23)- | −0.082881696 | −0.0385482 | 0.19327526 | 0.07272357 | −0.0911254 | 0.16860433 | 0.06307178 | −0.008118 | 0.23508166 |
| FGFR3 | | | | | | | | | |
| TGFB3)- | 0.009205087 | 0.2365879 | 0.30660575 | −0.0247737 | −0.0748315 | 0.02794234 | 0.13427449 | 0.04678231 | 0.41780093 |
| ENG | | | | | | | | | |
| TGFB3)- | −0.218614741 | 0.3072432 | 0.22931375 | −0.1337088 | −0.117146 | 0.07463971 | 0.07004593 | −0.3031937 | 0.04535182 |
| ITGB1 | | | | | | | | | |
| TGFB3)- | 0.114087432 | 0.02140543 | 0.07955083 | −0.0833408 | −0.0938475 | 0.03148732 | 0.14124571 | 0.08872062 | −0.0346187 |
| TGFBR2 | | | | | | | | | |
| TGFB3)- | 0.109666396 | −0.1981459 | −0.1647223 | 0.03672413 | −0.0658555 | 0.09156661 | 0.01057852 | 0.00821471 | 0.06234227 |
| ITGB3 | | | | | | | | | |
| TGFB3)- | −0.124126564 | 0.02106571 | 0.21453746 | 0.0461043 | −0.1416859 | 0.25081077 | 0.04042524 | 0.08985164 | 0.37839968 |
| ACVRL1 | | | | | | | | | |
| TGFB3)- | 0.106936332 | 0.04084233 | 0.1618438 | −0.0553203 | −0.1353651 | −0.1710069 | 0.17929309 | 0.19206388 | −0.1331232 |
| ITGB6 | | | | | | | | | |
| TGFB3)- | −0.175827991 | 0.15165741 | 0.22967181 | −0.0893175 | −0.1282746 | 0.09990887 | 0.17745588 | −0.2721642 | 0.12489766 |
| ITGB5 | | | | | | | | | |
| LAPP)- | 0.139367493 | 0.22316576 | 0.20410599 | 0.0611629 | 0.01556829 | 0.09181855 | 0.19158485 | 0.26061913 | 0.52080404 |
| PTH1R | | | | | | | | | |
| LAPP)- | 0.087275999 | 0.1762173 | −0.0460581 | 0.29478388 | −0.0654704 | 0.09354446 | −0.0400485 | 0.09769784 | 0.35817621 |
| RAMP2 | | | | | | | | | |
| LAPP)- | 0.139443622 | 0.02888773 | −0.0732539 | 0.08143759 | 0.05093248 | 0.17442498 | −0.0702667 | 0.20096335 | 0.30705281 |
| VIPR1 | | | | | | | | | |
| LAPP)- | 0.027945718 | 0.33765006 | 0.14600972 | 0.145828 | 0.07462984 | 0.21182266 | 0.2156808 | 0.24357325 | 0.41716526 |
| CALCR | | | | | | | | | |
| LAPP)- | 0.065893211 | 0.29244795 | 0.15498828 | 0.06086694 | −0.1133902 | 0.11701261 | 0.19715504 | 0.09875154 | 0.49073749 |
| ADRB2 | | | | | | | | | |
| LAPP)- | −0.108009275 | 0.02166033 | 0.07466044 | −0.1617238 | −0.1399776 | 0.08247933 | −0.0074337 | −0.1356173 | −0.0633264 |
| RAMP1 | | | | | | | | | |
| LAPP)- | 0.09187623 | 0.16905041 | 0.14283139 | 0.1693327 | 0.0736203 | 0.05412214 | 0.18049295 | 0.11917233 | 0.47738564 |
| ADRB3 | | | | | | | | | |
| LAPP)- | 0.20475193 | 0.20685498 | 0.00684196 | 0.1233794 | 0.12344465 | −0.0006975 | 0.18745505 | 0.15372874 | 0.52562647 |
| GPR84 | | | | | | | | | |
| TNFSF10)- | 0.103772383 | 0.23225562 | 0.01156828 | 0.10638454 | −0.0060125 | 0.07337933 | 0.11758104 | 0.03583341 | −0.1240573 |
| TNFRSF10D | | | | | | | | | |
| TNFSF10)- | 0.082989217 | 0.07839065 | 0.04157893 | −0.0267738 | −0.1764608 | −0.0002242 | 0.18775433 | 0.03641093 | −0.0285464 |
| RIPK1 | | | | | | | | | |
| TNFSF10)- | −0.049240743 | 0.07227834 | 0.14979126 | −0.0881331 | 0.1998529 | 0.12354209 | 0.05802171 | 0.16106991 | −0.1037513 |
| TNFRSF10B | | | | | | | | | |
| TNFSF10)- | 0.06882154 | 0.28040855 | 0.09539454 | 0.08890185 | 0.07970725 | 0.10437856 | 0.27770807 | 0.18979018 | −0.1208112 |
| TNFRSF10C | | | | | | | | | |
| TNFSF10)- | 0.015812998 | 0.13216836 | 0.04318563 | 0.02638276 | 0.11530032 | 0.0922377 | −0.0911132 | −0.0817323 | 0.05191104 |
| TNFRSF11B | | | | | | | | | |
| INHBA)- | −0.240970024 | 0.00932113 | 0.12071677 | −0.0692038 | −0.0726394 | −0.0041073 | 0.01178096 | −0.1724298 | 0.0027067 |
| ACVR1 | | | | | | | | | |
| INHBA)- | −0.31941397 | 0.12742859 | 0.19894655 | −0.2487655 | 0.05445601 | 0.05868496 | 0.10304582 | −0.2045485 | 0.24269463 |
| ENG | | | | | | | | | |
| INHBA)- | 0.062693703 | −0.0621527 | 0.16013264 | 0.16714276 | −0.3586241 | −0.2064402 | 0.38363111 | 0.27590268 | −0.3062463 |
| SMAD3 | | | | | | | | | |
| INHBA)- | 0.064507721 | 0.0352311 | −0.0787917 | −0.0770894 | 0.04517755 | −0.1763928 | −0.0624245 | 0.19153339 | 0.16788014 |
| BAMBI | | | | | | | | | |
| INHBA)- | −0.080977045 | 0.09636045 | 0.01323572 | 0.28789027 | 0.00517728 | −0.0406292 | −0.0125707 | −0.0142022 | −0.0164922 |
| ACVR1B | | | | | | | | | |
| INHA)- | −0.003041467 | 0.04038537 | 0.14631869 | 0.05005749 | 0.14253747 | 0.11938091 | −0.0405668 | 0.12187676 | −0.182114 |
| ACVR1 | | | | | | | | | |
| PI3)- PLD2 | 0.060780486 | −0.0271947 | 0.02712267 | 0.04590807 | 0.00717503 | 0.04888306 | 0.20316887 | 0.17342112 | 0.36981673 |
| LYPD3)- | 0.033982482 | −0.1191967 | 0.03007382 | −0.0206943 | −0.0368911 | 0.05849365 | 0.16721177 | 0.03964256 | −0.2800527 |
| AGR2 | | | | | | | | | |
| EFNB2)- | 0.147030299 | 0.16645232 | 0.06447908 | −0.1305836 | 0.13159056 | 0.14813599 | 0.15012651 | −0.0726728 | 0.03920182 |
| EPHB6 | | | | | | | | | |
| EFNB2)- | 0.216203548 | 0.28792067 | −0.2984242 | 0.18036591 | −0.2341866 | 0.30050463 | 0.07526189 | −0.0947777 | −0.0660212 |
| PECAM1 | | | | | | | | | |
| EFNB2)- | 0.263314013 | −0.1582521 | 0.14992947 | −0.1123726 | 0.31234912 | 0.2183621 | −0.0090726 | 0.00355334 | −0.0380172 |
| EPHB4 | | | | | | | | | |
| EFNB2)- | 0.231282887 | 0.25086203 | 0.11565187 | −0.0058182 | 0.00851478 | 0.07170688 | −0.0197645 | −0.2119135 | −0.0075782 |
| RHBDL2 | | | | | | | | | |
| EFNB2)- | 0.03744971 | 0.16305766 | 0.14480715 | 0.06671588 | 0.03861005 | 0.20312892 | −0.0195671 | 0.04065942 | −0.0996886 |
| EPHA3 | | | | | | | | | |
| EFNB2)- | 0.045339451 | 0.18235403 | 0.15532774 | 0.05153152 | −0.0394413 | 0.06934389 | −0.0682862 | −0.0211565 | 0.00235226 |
| EPHB1 | | | | | | | | | |
| EFNB2)- | 0.074337102 | 0.13252389 | 0.11169173 | 0.03766294 | 0.16375988 | 0.04142435 | −0.0251532 | −0.0506985 | −0.1323741 |
| EPHB3 | | | | | | | | | |
| EFNB2)- | 0.011186315 | 0.0349968 | 0.35054956 | −0.0166196 | 0.18404031 | 0.03978981 | −0.1045564 | −0.0920562 | 0.12160599 |
| EPHB2 | | | | | | | | | |
| EFNB2)- | 0.043623861 | −0.1976499 | 0.06899466 | −0.2492698 | 0.08142535 | 0.24248271 | 0.17575472 | −0.2104013 | 0.39137624 |
| EPHA4 | | | | | | | | | |
| BMP4)- | 0.036865786 | 0.08162661 | 0.08053853 | 0.02450092 | 0.04413437 | 0.27271209 | −0.1294572 | −0.121327 | −0.2560831 |
| LRP6 | | | | | | | | | |
| BMP4)- | −0.232398121 | 0.02353031 | 0.08424349 | −0.0394957 | −0.1244983 | −0.0037293 | 0.13502667 | 0.14356381 | 0.19967426 |
| BMPR2 | | | | | | | | | |
| BMP4)- | −0.134105141 | −0.0178528 | −0.0090113 | 0.06372985 | 0.12035855 | −0.1099631 | −0.0289442 | −0.1889814 | −0.2884564 |
| BMPR1A | | | | | | | | | |
| BMP4)- | −0.028637379 | 0.14621528 | 0.18922516 | −0.1708945 | −0.0807546 | 0.15844121 | −0.0604859 | −0.174201 | −0.2543819 |
| BMPR1B | | | | | | | | | |
| TNFSF9)- | 0.088520663 | 0.17193664 | 0.3168945 | 0.18503658 | 0.0530899 | 0.29182258 | 0.10288495 | 0.15896853 | 0.37101775 |
| TNFRSF9 | | | | | | | | | |
| TNFSF9)- | −0.058287207 | 0.12209037 | −0.0412474 | −0.0517337 | 0.04084154 | −0.0377005 | −0.0429243 | 0.1196757 | −0.0308049 |
| PVR | | | | | | | | | |
| TNFSF9)- | −0.092380952 | 0.05613417 | −0.0073736 | −0.0556572 | 0.00818064 | 0.29025216 | −0.0791758 | 0.03429 | 0.00208863 |
| TRAF2 | | | | | | | | | |
| C3)- | −0.148057335 | −0.1505802 | −0.0653546 | 0.09781061 | 0.13298682 | −0.0387301 | −0.1625584 | 0.01561681 | 0.0725595 |
| ITGAM | | | | | | | | | |
| C3)- CD46 | 0.145711156 | 0.18205839 | 0.05339891 | 0.10999048 | −0.048123 | −0.054011 | 0.12039511 | 0.0995149 | −0.085753 |
| C3)- C5AR2 | −0.080762346 | −0.2858589 | −0.1678323 | 0.08741 | 0.04707279 | 0.07009685 | −0.0678763 | 0.03608394 | 0.06901208 |
| C3)- CR2 | 0.049048406 | −0.1226104 | −0.1389682 | 0.04475723 | 0.07916655 | −0.2313619 | 0.0317011 | 0.01288319 | −0.056448 |
| C3)- | −0.21251082 | −0.1625461 | −0.0059882 | 0.16928598 | 0.03951644 | 0.11310179 | 0.16840016 | −0.1002547 | −0.0932286 |
| ITGAX | | | | | | | | | |
| C3)- C3AR1 | −0.222907079 | −0.2501567 | 0.05158507 | 0.06960088 | 0.02049895 | −0.0712017 | −0.084963 | 0.11514406 | 0.16104237 |
| C3)- | −0.103476913 | 0.34196319 | −0.0096609 | −0.2057274 | 0.21960416 | 0.03477343 | 0.1031505 | 0.00123034 | −0.2382453 |
| ADRA2A | | | | | | | | | |
| C3)- | 0.124416478 | −0.0792533 | 0.28892616 | 0.07111534 | 0.04487819 | 0.16371397 | −0.0720187 | −0.0607929 | 0.37883155 |
| IFITM1 | | | | | | | | | |
| C3)- CR1 | −0.161172167 | −0.2434427 | −0.0131976 | 0.03142878 | 0.02640684 | −0.0178594 | 0.13115886 | 0.05032516 | −0.041321 |
| C3)- ITGB2 | −0.220249961 | −0.2930657 | 0.11675025 | 0.09800073 | 0.12071848 | 0.16110166 | −0.045273 | 0.07479447 | 0.12853276 |
| C3)- CD81 | 0.149097908 | −0.0231388 | 0.36599677 | 0.05955035 | 0.08633996 | 0.02533145 | −0.1587474 | 0.03572904 | 0.2813496 |
| VASP)- | −0.135629915 | 0.00020505 | 0.24576808 | −0.0174383 | 0.16622503 | −0.1882188 | −0.2206067 | −0.1076261 | −0.4212605 |
| CXCR2 | | | | | | | | | |
| SLURP1)- | 0.098241651 | −0.0193041 | 0.16573372 | 0.09383135 | −0.0283575 | 0.06108676 | 0.07176021 | 0.09212225 | 0.26335555 |
| CHRNA7 | | | | | | | | | |
| TPH1)- | 0.032825469 | 0.12566409 | 0.16830751 | 0.1757831 | 0.11497879 | 0.22922968 | 0.2137474 | 0.01120689 | 0.36366992 |
| HTR1A | | | | | | | | | |
| TPH1)- | 0.060069221 | 0.12122369 | 0.08802128 | 0.34001551 | 0.07134473 | 0.05441153 | 0.24918985 | 0.14125848 | 0.4018572 |
| HTR1F | | | | | | | | | |
| TPH1)- | 0.101693112 | 0.07051021 | 0.19407204 | 0.18667999 | 0.18222348 | 0.09959415 | 0.30198372 | 0.04095528 | 0.34429197 |
| HTR1D | | | | | | | | | |
| TPH1)- | 0.177089285 | 0.16193911 | 0.27781452 | 0.14654842 | 0.02913152 | 0.23749154 | 0.10167101 | 0.14199929 | 0.51526838 |
| HTR2C | | | | | | | | | |
| TPH1)- | 0.045943978 | 0.27992314 | 0.29932478 | 0.13006218 | 0.13009022 | 0.1468671 | 0.23069352 | 0.05232241 | 0.28607878 |
| HTR1B | | | | | | | | | |
| TPH1)- | 0.066286322 | 0.04707611 | −0.0694316 | −0.0150495 | 0.14494325 | 0.13205121 | 0.04481442 | 0.11000204 | 0.04125961 |
| HTR2A | | | | | | | | | |
| TPH1)- | −0.074562783 | 0.07026966 | 0.16588874 | 0.04717454 | −0.0268383 | 0.14297772 | 0.14352041 | −0.164808 | 0.17868008 |
| HTR2B | | | | | | | | | |
| TPH1)- | 0.18497441 | 0.11976272 | 0.13549069 | 0.1383187 | 0.01628221 | 0.10056171 | 0.15707637 | 0.19778939 | 0.41296875 |
| HTR1E | | | | | | | | | |
| LMAN1)- | −0.205450513 | 0.10256564 | 0.08323201 | 0.06768523 | 0.1349292 | 0.2881171 | 0.15921276 | 0.15538619 | 0.29700812 |
| MCFD2 | | | | | | | | | |
| APOE)- | 0.074244561 | −0.2466813 | −0.0438378 | 0.07301073 | 0.08305159 | −0.0893963 | 0.07173692 | −0.1896038 | 0.1513409 |
| LRP6 | | | | | | | | | |
| APOE)- | −0.313479904 | 0.3471311 | −0.3428111 | −0.1937374 | 0.30188795 | 0.31054998 | 0.41506787 | 0.41515867 | −0.3222966 |
| LDLR | | | | | | | | | |
| APOE)- | −0.211635585 | 0.2450766 | −0.1840868 | −0.2963299 | 0.30479469 | 0.25390995 | 0.37181282 | 0.26202678 | −0.4081905 |
| LRP5 | | | | | | | | | |
| APOE)- | −0.19692435 | −0.3465745 | −0.1088687 | 0.17817089 | −0.0177315 | 0.12043357 | 0.0021642 | 0.22506878 | 0.3257825 |
| CHRNA4 | | | | | | | | | |
| APOE)- | −0.290742762 | −0.4338903 | 0.38749225 | 0.25801207 | −0.1779334 | 0.2358423 | −0.182133 | 0.26369956 | 0.40407644 |
| TREM2 | | | | | | | | | |
| APOE)- | −0.244237397 | 0.2071007 | −0.2888061 | −0.0899567 | 0.39702322 | 0.35652529 | 0.52032363 | 0.45270904 | −0.218873 |
| LSR | | | | | | | | | |
| APOE)- | −0.19343588 | 0.05125381 | 0.12264148 | 0.01188429 | −0.1236835 | 0.3677559 | −0.1912569 | 0.39571884 | −0.0304416 |
| SORL1 | | | | | | | | | |
| APOE)- | 0.076249388 | −0.1358158 | 0.22258244 | 0.05669873 | −0.1510254 | −0.0140188 | −0.1980514 | −0.0442902 | 0.21299744 |
| ABCA1 | | | | | | | | | |
| APOE)- | −0.134201525 | 0.01284864 | 0.21965212 | 0.02442775 | −0.1274707 | 0.19018274 | −0.2732323 | 0.2254393 | 0.07316598 |
| SCARB1 | | | | | | | | | |
| APOE)- | 0.17577661 | −0.2427463 | 0.09786025 | 0.40614221 | 0.21434891 | −0.2742955 | 0.19497341 | −0.3674122 | 0.55015584 |
| SDC2 | | | | | | | | | |
| APOE)- | −0.013414313 | −0.0721076 | −0.2296423 | 0.00826076 | 0.11433898 | −0.2303776 | 0.10272478 | −0.1948687 | 0.10453067 |
| LRP8 | | | | | | | | | |
| APOC2)- | 0.030160688 | −0.0196261 | 0.03097826 | −0.1065534 | 0.0531609 | 0.02804249 | 0.19227508 | 0.16277677 | −0.2688275 |
| LDLR | | | | | | | | | |
| GDF15)- | −0.162487073 | 0.16045701 | 0.18578724 | 0.16263138 | 0.24979888 | −0.121927 | −0.1328356 | 0.06999773 | −0.1626176 |
| GFRAL | | | | | | | | | |
| GDF15)- | 0.019361645 | 0.16917141 | 0.09367368 | −0.022593 | −0.062998 | 0.20023443 | −0.0494518 | −0.0097498 | −0.0957368 |
| RET | | | | | | | | | |
| LAMA5)- | 0.033022889 | 0.02205293 | 0.21128238 | 0.01051219 | −0.1545152 | −0.1981462 | 0.13177954 | 0.02378787 | 0.38901843 |
| SDC1 | | | | | | | | | |
| LAMA5)- | 0.367568201 | −0.1360052 | 0.25986451 | −0.1929331 | 0.24082257 | 0.29672713 | 0.13351339 | 0.10410283 | −0.0941256 |
| ITGA6 | | | | | | | | | |
| LAMA5)- | 0.113764833 | −0.0062837 | 0.19732256 | −0.0147467 | 0.10951069 | 0.39108743 | 0.06538518 | −0.0740634 | 0.17293158 |
| BCAM | | | | | | | | | |
| LAMA5)- | −0.129207081 | 0.17542081 | 0.18718225 | 0.10022058 | 0.13137455 | −0.2019325 | 0.16018209 | −0.1306821 | 0.45748718 |
| ITGB1 | | | | | | | | | |
| LAMA5)- | 0.133387041 | 0.07787346 | 0.03098768 | 0.02878431 | 0.11654336 | 0.18843588 | −0.0340507 | −0.0807701 | 0.26868557 |
| ITGA2 | | | | | | | | | |
| F12)- | 0.200906004 | 0.26361275 | 0.19473463 | 0.2291634 | 0.09179259 | 0.19207191 | 0.09409485 | 0.25635403 | 0.3107759 |
| GP1BA | | | | | | | | | |
| JAG1)- | 0.060456655 | −0.1194925 | 0.18540978 | −0.0721294 | 0.14527532 | 0.24766787 | −0.0399974 | −0.0416788 | 0.00912493 |
| CD46 | | | | | | | | | |
| JAG1)- | −0.004827124 | −0.0163087 | 0.18031473 | −0.0409788 | −0.0385999 | 0.13956217 | 0.02754354 | −0.2178521 | −0.0338223 |
| NOTCH4 | | | | | | | | | |
| JAG1)- | −0.068988539 | −0.0766166 | 0.15380423 | −0.0016361 | 0.00432425 | 0.34149614 | −0.0547138 | −0.200619 | 0.17447077 |
| NOTCH1 | | | | | | | | | |
| JAG1)- | −0.04168394 | −0.0310764 | −0.0402541 | 0.06387451 | 0.01054921 | 0.21469397 | −0.2250987 | −0.0955058 | −0.0482083 |
| NOTCH2 | | | | | | | | | |
| JAG1)- | −0.140174664 | 0.08592304 | 0.22479372 | −0.1560495 | −0.1008205 | 0.38745322 | 0.03480837 | −0.1039701 | 0.07724992 |
| NOTCH3 | | | | | | | | | |
| CRP)- | −0.188117004 | −0.1117774 | 0.22545293 | 0.18496994 | −0.1367095 | 0.12851105 | −0.0049117 | 0.11256886 | 0.32833053 |
| OLR1 | | | | | | | | | |
| CRP)- CR1 | −0.037391399 | 0.061006 | 0.14693096 | 0.18463004 | 0.09055912 | 0.07123143 | 0.15633858 | 0.04242125 | 0.36910382 |
| TSHB)- | 0.115892512 | 0.18770225 | 0.18804035 | 0.23343294 | 0.0581832 | 0.10822721 | 0.18128548 | 0.31071995 | 0.58952765 |
| PTH1R | | | | | | | | | |
| TSHB)- | −0.031779521 | 0.00205964 | 0.07148543 | 0.14554321 | −0.0047706 | 0.30132884 | −0.0592567 | 0.1388635 | 0.23628665 |
| VIPR1 | | | | | | | | | |
| TSHB)- | 0.081374955 | 0.21914172 | 0.15470992 | 0.15043066 | −0.0090129 | 0.25664367 | 0.12398449 | 0.27873572 | 0.53963823 |
| ADRB2 | | | | | | | | | |
| TSHB)- | −0.048778344 | 0.14123886 | 0.23799392 | 0.33291147 | 0.12318878 | 0.25759705 | 0.26880923 | 0.09575131 | 0.57325229 |
| ADRB3 | | | | | | | | | |
| TSHB)- | −0.004250157 | 0.25579072 | 0.19305505 | 0.25028601 | 0.01536895 | 0.17812534 | 0.05567707 | 0.23831936 | 0.50332992 |
| GPR84 | | | | | | | | | |
| FST)- | −0.018425475 | −0.0693492 | −0.0591719 | −0.077035 | 0.03019538 | −0.0002022 | −0.0972873 | −0.0786516 | −0.176082 |
| BMPR2 | | | | | | | | | |
| FST)- | 0.214451406 | 0.12158964 | 0.01423524 | 0.13563925 | 0.10854079 | 0.13281511 | 0.06730651 | 0.25901213 | 0.49861442 |
| BMPR1B | | | | | | | | | |
| GRP)- | 0.200487077 | 0.15930201 | 0.08251422 | 0.03705299 | −0.0707861 | 0.08644372 | 0.17454152 | 0.25243077 | 0.46461584 |
| GRPR | | | | | | | | | |
| SEMA6A)- | −0.000927447 | −0.0159369 | 0.00945344 | −0.0120243 | −0.1041309 | 0.19579718 | 0.10246962 | 0.02796311 | 0.19048526 |
| PLXNA4 | | | | | | | | | |
| SEMA6A)- | −0.004568765 | −0.0791812 | 0.22524839 | 0.07577782 | 0.11666611 | 0.13214065 | 0.11877491 | 0.05539753 | −0.2343243 |
| PLXNA2 | | | | | | | | | |
| LACRT)- | −0.022817194 | −0.1177449 | 0.17538419 | −0.0995121 | −0.0174437 | 0.09087393 | 0.00834898 | 0.1165353 | −0.3289818 |
| SDC1 | | | | | | | | | |
| GDF11)- | −0.084694479 | −0.1317156 | 0.07368527 | −0.004582 | 0.00874421 | 0.21496344 | 0.05736358 | 0.03194319 | −0.2059996 |
| ACVR1B | | | | | | | | | |
| GDF11)- | −0.155132984 | −0.1204004 | −0.0152167 | −0.0918094 | −0.1773147 | 0.25924727 | −0.1117852 | −0.0284583 | −0.1026789 |
| BMPR2 | | | | | | | | | |
| GDF11)- | −0.027088987 | 0.11879264 | 0.20695394 | 0.10559328 | 0.00366388 | 0.14135099 | 0.09516233 | 0.09997217 | 0.39930516 |
| BMPR1A | | | | | | | | | |
| GDF11)- | 0.031363309 | 0.12713028 | 0.13703025 | 0.13015016 | −0.0270361 | 0.16392831 | 0.11571961 | 0.21241884 | 0.37520139 |
| BMPR1B | | | | | | | | | |
| LAMC1)- | 0.108138985 | 0.13732845 | 0.08023423 | −0.047931 | −0.0950036 | −0.1730079 | 0.10612931 | 0.20961052 | 0.051313 |
| ITGA6 | | | | | | | | | |
| LAMC1)- | −0.316310815 | 0.20814354 | 0.29569347 | −0.1306928 | −0.0625894 | 0.22014162 | 0.188347 | −0.2815284 | 0.16220555 |
| ITGA1 | | | | | | | | | |
| LAMC1)- | 0.038402546 | −0.0603912 | 0.00190791 | 0.03557775 | 0.02358427 | −0.0989933 | 0.01417301 | 0.0155198 | −0.0580417 |
| ITGA7 | | | | | | | | | |
| LAMC1)- | −0.177757597 | 0.13802982 | 0.29594808 | 0.06104006 | −0.0565049 | 0.56375991 | 0.26512913 | −0.2968657 | 0.31484932 |
| ITGAV | | | | | | | | | |
| LAMC1)- | −0.174091607 | 0.29776639 | 0.42876832 | −0.3017235 | −0.1251941 | 0.44685488 | 0.21285035 | −0.2947367 | 0.34082997 |
| ITGB1 | | | | | | | | | |
| LAMC1)- | 0.062955981 | −0.0283354 | 0.13805073 | 0.22209951 | −0.2221625 | −0.2355986 | 0.06662761 | 0.06799154 | 0.28101062 |
| ITGA2 | | | | | | | | | |
| IL1RN)- | 0.103051836 | −0.1937831 | −0.1659669 | −0.0390897 | −0.0451928 | −0.0077969 | −0.032222 | 0.10011866 | 0.04096142 |
| IL1R1 | | | | | | | | | |
| IL1RN)- | −0.259004865 | 0.01251009 | 0.276762 | 0.10315431 | −0.088261 | 0.12499469 | −0.2103818 | 0.19384568 | 0.23700711 |
| IL1R2 | | | | | | | | | |
| ORM1)- | −0.014398498 | 0.10650868 | 0.20953127 | 0.21463537 | 0.0094068 | 0.19359977 | 0.02599702 | 0.27175426 | 0.44516767 |
| CCR5 | | | | | | | | | |
| CCN3)- | −0.002506504 | 0.02777347 | −0.1042146 | 0.09307075 | 0.12749614 | 0.17366215 | 0.02359898 | 0.15589411 | 0.05852543 |
| PLXNA1 | | | | | | | | | |
| CCN3)- | 0.091950474 | 0.04336008 | 0.09724769 | −0.2088231 | 0.08942764 | 0.13051889 | 0.00391553 | 0.12545631 | 0.07208265 |
| NOTCH1 | | | | | | | | | |
| CCL21)- | −0.131528484 | −0.0200629 | −0.1241323 | −0.1946415 | 0.18708534 | 0.20719096 | 0.14761927 | 0.0253189 | −0.1722517 |
| ADRA2A | | | | | | | | | |
| TGS1)- | −0.100336686 | −0.2051466 | 0.18628119 | 0.18782833 | 0.02835043 | 0.12860954 | −0.1298319 | −0.0797366 | 0.10253128 |
| RXRA | | | | | | | | | |
| MMP7)- | 0.053759141 | −0.0469278 | −0.0456598 | 0.04038398 | −0.1233189 | −0.052248 | 0.04661041 | 0.27983051 | 0.0623009 |
| CD44 | | | | | | | | | |
| MMP7)- | 0.154175163 | −0.0855251 | 0.23171173 | −0.0003647 | 0.20174187 | 0.19238933 | −0.1484505 | −0.1270448 | 0.00360749 |
| SDC1 | | | | | | | | | |
| MMP7)- | 0.353031641 | −0.1118039 | 0.38421474 | −0.2309797 | 0.42239646 | 0.46530471 | −0.1442193 | −0.1581723 | 0.28546655 |
| CD151 | | | | | | | | | |
| THBS1)- | 0.028060544 | 0.05406965 | 0.10793875 | −0.2564663 | −0.0673329 | −0.0349352 | 0.04515804 | 0.04481355 | 0.23723729 |
| ITGA4 | | | | | | | | | |
| THBS1)- | 0.294735424 | −0.1561633 | 0.36184056 | 0.26771598 | −0.2460216 | −0.2644669 | 0.3226982 | 0.30604234 | −0.2034833 |
| CD47 | | | | | | | | | |
| THBS1)- | −0.017672399 | −0.0624976 | 0.0966358 | 0.05401989 | 0.17035983 | 0.32915231 | 0.18992852 | 0.05826821 | −0.1574373 |
| SDC1 | | | | | | | | | |
| THBS1)- | −0.23673476 | 0.24449688 | 0.3575833 | −0.1793029 | −0.0159857 | 0.57632553 | 0.23802493 | −0.394859 | 0.32193582 |
| ITGB1 | | | | | | | | | |
| THBS1)- | 0.081526778 | −0.1349376 | 0.19546412 | 0.35067421 | −0.0868235 | 0.01980288 | 0.22570657 | 0.09881845 | −0.3851129 |
| LRP5 | | | | | | | | | |
| THBS1)- | 0.061946089 | 0.02007763 | −0.0648802 | −0.2273594 | −0.0116939 | −0.238879 | −0.0672424 | 0.18298657 | 0.15227198 |
| ITGA2B | | | | | | | | | |
| THBS1)- | 0.049566849 | −0.0556811 | 0.03066724 | −0.2215198 | 0.02253132 | −0.0603767 | 0.08297565 | 0.02800857 | 0.15294099 |
| ITGB3 | | | | | | | | | |
| THBS1)- | 0.225388502 | −0.0777387 | 0.20989235 | 0.13940149 | −0.0993154 | −0.0743188 | 0.32281121 | 0.34247867 | −0.2389539 |
| SDC4 | | | | | | | | | |
| THBS1)- | 0.070497778 | 0.09909088 | −0.0522011 | 0.09792876 | 0.17630277 | −0.0853707 | −0.0764477 | 0.14206154 | 0.11660215 |
| SCARB1 | | | | | | | | | |
| THBS1)- | 0.238210179 | −0.0837819 | 0.22723398 | 0.11478865 | −0.1262161 | −0.2004002 | 0.16292885 | 0.23613259 | −0.1278182 |
| ITGA6 | | | | | | | | | |
| THBS1)- | 0.00141509 | 0.19556085 | 0.00699 | 0.03545482 | −0.0680368 | −0.0914337 | 0.02146544 | 0.03996519 | 0.36826287 |
| TNFRSF11B | | | | | | | | | |
| THBS1)- | 0.044665511 | 0.03608506 | −0.01859 | −0.0642653 | −0.0143751 | −0.2070796 | 0.01992035 | 0.05255078 | 0.2337576 |
| CD36 | | | | | | | | | |
| ADAM10)- | −0.05689142 | −0.0367214 | 0.11674956 | 0.05518898 | 0.03939489 | 0.028368 | 0.022512 | 0.06584577 | −0.0519135 |
| GPNMB | | | | | | | | | |
| ADAM10)- | 0.092514376 | 0.10628032 | 0.11209117 | 0.08666244 | −0.0176284 | 0.16562704 | 0.03538249 | −0.2045507 | 0.09167378 |
| TSPAN5 | | | | | | | | | |
| ADAM10)- | 0.101450549 | −0.2136709 | 0.18392278 | −0.0642718 | 0.26957914 | 0.25657201 | −0.0795257 | −0.1723442 | 0.17334386 |
| TSPAN15 | | | | | | | | | |
| ADAM10)- | −0.176134383 | −0.1180886 | −0.0823651 | −0.0819457 | 0.03110806 | 0.06861147 | 0.01812448 | 0.135801 | 0.18764674 |
| CD44 | | | | | | | | | |
| ADAM10)- | −0.210767877 | 0.09666812 | 0.31319993 | 0.04483965 | 0.09393056 | −0.1290672 | −0.1783145 | 0.13740834 | −0.1448788 |
| CADM1 | | | | | | | | | |
| ADAM10)- | 0.173995268 | 0.06673219 | 0.13751233 | −0.1343946 | 0.15496826 | 0.22912132 | −0.060051 | −0.1261953 | 0.1430804 |
| TSPAN14 | | | | | | | | | |
| ADAM10)- | 0.030117347 | −0.0034466 | 0.20131258 | −0.0258078 | 0.09305923 | 0.05404235 | −0.2217256 | 0.05919598 | −0.0558598 |
| NOTCH1 | | | | | | | | | |
| ADAM10)- | 0.071162265 | 0.08848624 | 0.0206098 | 0.10862083 | 0.03829421 | −0.0148234 | −0.0223999 | −0.0567858 | 0.02083931 |
| IL6R | | | | | | | | | |
| ADAM10)- | 0.050153581 | −0.105419 | 0.26435606 | −0.1588567 | 0.18138837 | 0.30740985 | −0.2344281 | −0.2346436 | 0.40597762 |
| MET | | | | | | | | | |
| ADAM10)- | 0.054973857 | 0.04406442 | 0.19704893 | −0.0696881 | 0.08431329 | 0.07016976 | −0.1040829 | −0.0245626 | 0.27141493 |
| TSPAN17 | | | | | | | | | |
| ADAM10)- | −0.10389053 | 0.37273548 | 0.11697154 | 0.13818124 | 0.07147588 | −0.00718 | −0.0563978 | 0.09295955 | −0.2295404 |
| EPHA3 | | | | | | | | | |
| ADAM10)- | 0.102769827 | 0.11263381 | 0.08100519 | 0.041588 | −0.0888949 | 0.10451325 | 0.13354057 | −0.151118 | −0.1607438 |
| TREM2 | | | | | | | | | |
| ADAM10)- | −0.222018773 | 0.22797481 | 0.08894748 | 0.15109324 | 0.0476307 | −0.1175347 | 0.05991171 | 0.14590569 | −0.1621531 |
| AXL | | | | | | | | | |
| COL8A1)- | −0.114885598 | 0.12379641 | 0.2403315 | −0.0225916 | −0.1573676 | 0.02458439 | 0.23091864 | −0.1469775 | 0.22981731 |
| ITGA1 | | | | | | | | | |
| COL8A1)- | −0.024897068 | −0.201008 | 0.08614293 | 0.15044307 | −0.1481332 | −0.0698069 | 0.08177224 | 0.03207115 | −0.2341208 |
| ITGA2 | | | | | | | | | |
| LYZ)- | 0.084432981 | 0.14280923 | 0.03666689 | 0.16661711 | −0.0628007 | −0.0214083 | 0.14159325 | −0.2568962 | −0.2201037 |
| ITGAL | | | | | | | | | |
| VWF)- | −0.082454794 | −0.2664514 | −0.0580773 | 0.36251245 | 0.09405437 | 0.14316465 | 0.17663709 | 0.18963535 | 0.34539042 |
| ITGA2B | | | | | | | | | |
| VWF)- | −0.021202409 | −0.0996642 | −0.0851713 | 0.24767276 | −0.0169145 | 0.151213 | 0.0299128 | −0.0371025 | 0.23694575 |
| ITGB3 | | | | | | | | | |
| VWF)- | −0.17408775 | −0.0018261 | −0.0363485 | 0.20740055 | 0.17913104 | 0.16374422 | 0.14923424 | 0.15526301 | 0.4717884 |
| STAB2 | | | | | | | | | |
| VWF)- | 0.010749363 | −0.11739 | −0.007743 | 0.32383828 | 0.13699511 | 0.09438073 | 0.17324935 | 0.18338861 | 0.37511919 |
| SELP | | | | | | | | | |
| VWF)- | −0.031296404 | −0.2073002 | 0.11540701 | 0.30155022 | −0.0690748 | 0.17252771 | −0.0659608 | 0.12689856 | 0.26487548 |
| SIRPA | | | | | | | | | |
| VWF)- | 0.070362713 | −0.0654732 | −0.0967193 | 0.16411201 | 0.10822116 | 0.062767 | 0.22799224 | 0.27981496 | 0.07553907 |
| TNFRSF11B | | | | | | | | | |
| VWF)- | 0.003606155 | −0.1492246 | −0.0722455 | 0.28122922 | 0.07050818 | 0.13980904 | 0.0183994 | 0.28574443 | 0.41001831 |
| GP1BA | | | | | | | | | |
| CDH1)- | 0.144112315 | 0.00367636 | 0.12536621 | −0.1999118 | 0.18077496 | 0.22147417 | −0.1865994 | 0.05954493 | 0.15047749 |
| EGFR | | | | | | | | | |
| CDH1)- | 0.153670393 | −0.2400387 | 0.27855276 | −0.1952954 | 0.28658808 | 0.31620791 | −0.1185995 | −0.0705035 | 0.3515399 |
| LRP5 | | | | | | | | | |
| CDH1)- | −0.189236137 | −0.1363166 | 0.03023468 | −0.115615 | 0.01709605 | −0.1156013 | 0.01776298 | 0.09118351 | −0.1502311 |
| IGFIR | | | | | | | | | |
| CDH1)- | 0.013868017 | −0.0520308 | −0.1716459 | −0.019067 | −0.294059 | 0.07964003 | 0.14033324 | −0.1910707 | −0.3488696 |
| ITGB7 | | | | | | | | | |
| CDH1)- | 0.447200329 | −0.34081 | 0.53449061 | −0.2813153 | 0.48826 | 0.53397115 | −0.3795533 | −0.4102675 | 0.41289991 |
| ERBB3 | | | | | | | | | |
| CDH1)- | 0.09915501 | 0.09674377 | −0.2823386 | 0.06231838 | −0.2647425 | 0.10087812 | 0.13841764 | −0.0361883 | −0.2805444 |
| KLRG1 | | | | | | | | | |
| CDH1)- | 0.012170013 | 0.11861425 | −0.0730366 | 0.1612916 | −0.0665077 | −0.0365628 | −0.1566902 | −0.0430656 | −0.3605126 |
| CDH2 | | | | | | | | | |
| CDH1)- | 0.167243144 | 0.03987978 | −0.1825948 | 0.09541385 | −0.3042123 | 0.22373898 | −0.053671 | −0.1362163 | −0.1822568 |
| ITGAE | | | | | | | | | |
| CDH1)- | −0.096919374 | 0.17778051 | 0.08059488 | 0.19540917 | 0.01723704 | −0.0746512 | 0.00630706 | 0.14385273 | −0.197501 |
| PTPRM | | | | | | | | | |
| CDH1)- | 0.39508632 | −0.0694164 | 0.50011717 | −0.109314 | 0.4558024 | 0.46898485 | −0.146519 | −0.1504395 | 0.29748018 |
| PTPRF | | | | | | | | | |
| SEMA7A)- | −0.066018075 | 0.04179168 | 0.11634352 | −0.0831578 | 0.00217394 | −0.041153 | −0.0175616 | −0.0358002 | 0.08772899 |
| ITGB1 | | | | | | | | | |
| SEMA7A)- | −0.064446095 | 0.1115422 | 0.16453306 | 0.03596971 | 0.02937648 | 0.03181937 | 0.089274 | −0.0889936 | 0.22174213 |
| ITGA1 | | | | | | | | | |
| RIMS2)- | 0.015655318 | 0.23397271 | −0.1640515 | 0.02024423 | 0.07933991 | −0.0107627 | −0.1246805 | −0.069082 | 0.34697034 |
| ABCA1 | | | | | | | | | |
| ANOS1)- | 0.051135987 | 0.19799229 | 0.01712057 | −0.1553885 | −0.1239624 | 0.20243288 | −0.0225741 | −0.2043415 | 0.38996297 |
| SDC2 | | | | | | | | | |
| ANOS1)- | 0.088910297 | 0.09001115 | −0.1144156 | −0.1265302 | −0.1298258 | 0.04811962 | 0.08034121 | −0.3668217 | 0.41369048 |
| FGFR1 | | | | | | | | | |
| TIMP2)- | 0.083289881 | 0.16682534 | −0.2505031 | −0.1088697 | 0.43235611 | 0.07423606 | −0.4186025 | 0.08794434 | 0.35021208 |
| CD44 | | | | | | | | | |
| TIMP2)- | −0.348775454 | 0.41576919 | 0.37357744 | −0.2681686 | −0.0920844 | 0.7404912 | 0.34990743 | −0.4597588 | 0.49823728 |
| ITGB1 | | | | | | | | | |
| LGALS3BP)- | −0.094758797 | 0.06905895 | 0.17145636 | −0.0307462 | 0.13868013 | 0.05461491 | −0.09276 | 0.13775295 | −0.0763647 |
| ITGB1 | | | | | | | | | |
| IL1A)- | 0.121390519 | −0.1594918 | −0.2194756 | −0.0725858 | −0.1755794 | −0.0785652 | −0.2336633 | −0.0801037 | −0.1087602 |
| IL1R1 | | | | | | | | | |
| IL1A)- | 0.114294129 | −0.1658734 | 0.1711724 | −0.0169804 | −0.141781 | 0.09281494 | 0.08071533 | 0.09372501 | 0.02897112 |
| IL1RAP | | | | | | | | | |
| IL1A)- | −0.080939152 | 0.14964175 | 0.04514158 | −0.0283939 | 0.02202002 | 0.21018252 | 0.13952099 | 0.07780024 | 0.16082338 |
| IL1R2 | | | | | | | | | |
| IL1B)- | −0.007333549 | −0.0435363 | 0.12637003 | 0.12217341 | −0.0540157 | 0.10773576 | 0.12667489 | 0.20329115 | 0.033538 |
| SIGIRR | | | | | | | | | |
| IL1B)- | 0.019521382 | 0.01462879 | −0.004302 | −0.1259875 | −0.175078 | 0.02314518 | −0.2055546 | −0.0831888 | −0.0062483 |
| IL1R1 | | | | | | | | | |
| IL1B)- | 0.237114931 | 0.1204384 | 0.04143185 | 0.17389929 | 0.01113475 | 0.12074647 | 0.02476935 | 0.16915178 | 0.39740461 |
| ADRB2 | | | | | | | | | |
| IL1B)- | −0.087406842 | −0.0342042 | 0.17235076 | −0.1270204 | −0.1008901 | 0.15309867 | 0.00959142 | 0.08183067 | −0.1532496 |
| IL1RAP | | | | | | | | | |
| IL1B)- | −0.102110325 | 0.10692627 | 0.15576239 | −0.0845767 | 0.04782075 | 0.18397576 | −0.136405 | 0.16801295 | 0.21594759 |
| IL1R2 | | | | | | | | | |
| LCN1)- | 0.036020999 | 0.10122139 | 0.16369498 | 0.20509741 | 0.00126037 | −0.0576203 | −0.1199807 | 0.0316686 | 0.20886061 |
| LMBR1L | | | | | | | | | |
| CNTN3)- | 0.084638356 | −0.0710468 | 0.03641904 | 0.03497529 | 0.00618691 | −0.0427475 | 0.19260368 | −0.1574927 | 0.27065647 |
| PTPRG | | | | | | | | | |
| IL7)- IL7R | 0.189572798 | 0.16373772 | −0.0548943 | 0.04561109 | 0.15606878 | −0.0032668 | 0.18309519 | 0.05512738 | 0.18925173 |
| IL7)- | 0.115599711 | 0.11041807 | −0.0115049 | 0.16486092 | 0.04001419 | 0.09636685 | 0.04603704 | 0.05692926 | 0.19315793 |
| IL2RG | | | | | | | | | |
| CKLF)- | −0.02377942 | −0.1002016 | 0.12183606 | 0.01745475 | −0.1107141 | 0.31229927 | −0.0747026 | 0.00984353 | 0.0825484 |
| CCR4 | | | | | | | | | |
| FARP2)- | 0.044253552 | −0.00544 | −0.0970274 | 0.13697978 | −0.0370361 | 0.00293026 | 0.09931448 | −0.0991177 | 0.1055739 |
| PLXNA3 | | | | | | | | | |
| FARP2)- | −0.082667615 | 0.08528188 | −0.0193913 | 0.03811849 | 0.18066758 | 0.14952127 | 0.00340583 | −0.0340723 | 0.01736519 |
| PLXNA1 | | | | | | | | | |
| FARP2)- | 0.07412459 | −0.2319082 | 0.12194127 | −0.0648065 | 0.08815333 | 0.04222273 | −0.2022357 | −0.1692396 | 0.19931043 |
| PLXNA2 | | | | | | | | | |
| FARP2)- | −0.057579304 | 0.12246464 | 0.10605315 | 0.1131212 | 0.09258573 | 0.14442098 | 0.03197578 | −0.0470102 | −0.2045847 |
| PLXNA4 | | | | | | | | | |
| LAMC2)- | 0.612644123 | −0.2564068 | 0.51711895 | −0.1870492 | 0.42539019 | 0.59772216 | −0.1160597 | −0.2269052 | 0.27036797 |
| ITGA6 | | | | | | | | | |
| LAMC2)- | 0.307559136 | 0.01523252 | 0.30176978 | 0.01475211 | 0.21637551 | 0.31036543 | −0.0470956 | −0.045387 | 0.05159722 |
| CD151 | | | | | | | | | |
| LAMC2)- | −0.15011754 | 0.14422242 | 0.23157457 | 0.1935464 | 0.30814703 | −0.15781 | −0.0916995 | 0.17569664 | 0.03206147 |
| ITGB1 | | | | | | | | | |
| LAMC2)- | 0.365150601 | −0.016703 | 0.35499963 | 0.01087074 | 0.30753277 | 0.42642208 | −0.1989249 | −0.2914526 | 0.4238506 |
| ITGA2 | | | | | | | | | |
| LAMC2)- | 0.23926251 | −0.0382716 | 0.32627462 | 0.03226066 | 0.22955987 | 0.2273694 | −0.21542 | −0.2649118 | 0.39710817 |
| COL17A1 | | | | | | | | | |
| NID1)- | −0.035384879 | 0.3312759 | 0.3011829 | −0.3904996 | −0.2059261 | 0.29538863 | 0.129567 | −0.2864192 | 0.17868034 |
| ITGB1 | | | | | | | | | |
| NID1)- | −0.022757582 | −0.187152 | −0.0543005 | 0.01982698 | −0.0932222 | 0.00205307 | 0.12459574 | −0.0949793 | 0.10503467 |
| ITGB3 | | | | | | | | | |
| NID1)- | 0.025820269 | 0.03835501 | 0.00611596 | −0.1849233 | 0.08326349 | −0.1026025 | 0.16145642 | 0.00616701 | 0.18789308 |
| COL13A1 | | | | | | | | | |
| NID1)- | 0.268889433 | −0.1202461 | 0.20268687 | 0.30775676 | −0.2067258 | −0.2920965 | 0.32482413 | 0.30725043 | −0.3306637 |
| PTPRF | | | | | | | | | |
| NMU)- | −0.040908019 | 0.13871233 | 0.23561342 | 0.15969554 | −0.0748995 | −0.0778091 | 0.04566602 | 0.27735505 | −0.1734488 |
| ADRA2A | | | | | | | | | |
| NMU)- | −0.058950046 | 0.0437271 | 0.1767571 | 0.13217193 | −0.1164095 | 0.2704182 | 0.14567633 | 0.14943529 | 0.44324928 |
| NMUR1 | | | | | | | | | |
| FGF2)- | 0.168063981 | −0.0158993 | 0.13260622 | −0.0860167 | 0.04721706 | −0.0319016 | 0.11949555 | −0.0162911 | −0.0712796 |
| SDC1 | | | | | | | | | |
| FGF2)- | −0.008823948 | −0.0584866 | −0.0751126 | 0.19962239 | 0.1281773 | −0.1635212 | 0.10928643 | −0.2923254 | 0.31049049 |
| FGFR1 | | | | | | | | | |
| FGF2)- | −0.009666225 | −0.2036428 | 0.01382623 | −0.0523738 | −0.0494275 | −0.1688614 | 0.01626769 | −0.0829091 | 0.10774353 |
| FGFR2 | | | | | | | | | |
| FGF2)- | 0.041809577 | 0.1646065 | −0.1868661 | 0.19319212 | −0.0501991 | −0.0147369 | −0.4137748 | −0.0599517 | 0.4563121 |
| CD44 | | | | | | | | | |
| FGF2)- | 0.09059334 | −0.0014124 | −0.2955434 | 0.07580975 | −0.0586425 | −0.0771981 | 0.09035596 | 0.00925047 | 0.07335021 |
| SDC3 | | | | | | | | | |
| FGF2)- | 0.238784332 | 0.08868095 | 0.03524729 | 0.09681152 | 0.06884339 | 0.07955952 | 0.26195163 | 0.27949827 | −0.0120307 |
| SDC4 | | | | | | | | | |
| FGF2)- | 0.052887371 | −0.075344 | −0.1300493 | 0.05437749 | 0.02048379 | −0.191477 | −0.0098721 | −0.0565804 | 0.118286 |
| SDC2 | | | | | | | | | |
| FGF2)- | 0.017074869 | 0.07647825 | 0.10776679 | 0.05330251 | −0.1345945 | −0.0698011 | −0.0247962 | −0.0736229 | 0.07519004 |
| FGFRL1 | | | | | | | | | |
| FGF2)- | 0.071153221 | 0.038884 | 0.31473047 | −0.0329953 | 0.01336469 | 0.28628707 | −0.1003315 | 0.11796634 | −0.0540875 |
| FGFR4 | | | | | | | | | |
| FGF2)- | 0.115797481 | −0.1359394 | 0.10094516 | −0.0918725 | −0.0525026 | −0.0717073 | 0.19083579 | −0.0720544 | −0.0331747 |
| FGFR3 | | | | | | | | | |
| FGF2)- | −0.062789653 | −0.0533209 | 0.04594141 | 0.07196938 | 0.06326926 | −0.0239255 | 0.02661565 | −0.0477308 | 0.41823508 |
| NRP1 | | | | | | | | | |
| IL11)- | 0.014879545 | −0.1023695 | 0.0342392 | 0.03264521 | −0.0784105 | 0.21092001 | 0.05158982 | 0.07511737 | −0.0177608 |
| IL11RA | | | | | | | | | |
| IL11)- | −0.072029778 | 0.21216801 | 0.0980021 | −0.0331368 | −0.1940135 | −0.019874 | −0.1917773 | 0.0776737 | 0.12664061 |
| IL6ST | | | | | | | | | |
| WNT5A)- | −0.012130721 | −0.0889183 | −0.1108838 | −0.2531649 | −0.2287705 | 0.20277289 | 0.11575611 | −0.076392 | 0.28313397 |
| PTK7 | | | | | | | | | |
| WNT5A)- | −0.130712526 | 0.04296044 | −0.0326811 | 0.17497323 | −0.1337437 | −0.1400639 | 0.19460417 | 0.16722373 | −0.1692306 |
| LDLR | | | | | | | | | |
| WNT5A)- | −0.027441938 | −0.0058342 | −0.0810012 | 0.04657885 | −0.1384203 | −0.1995019 | 0.13620219 | 0.14661382 | −0.343613 |
| LRP5 | | | | | | | | | |
| WNT5A)- | 0.089155553 | 0.13549121 | 0.00656248 | 0.05639195 | −0.0890889 | −0.1316997 | 0.23397243 | 0.1058209 | −0.0876032 |
| FZD5 | | | | | | | | | |
| WNT5A)- | −0.065724796 | 0.10941873 | 0.33181145 | −0.0479481 | −0.0705209 | 0.05760968 | 0.15904843 | −0.0362731 | 0.33834446 |
| ROR1 | | | | | | | | | |
| WNT5A)- | 0.034540669 | −0.0735045 | 0.05789387 | −0.0840869 | 0.0334432 | 0.07724253 | 0.09999166 | 0.06013349 | −0.0158332 |
| VANGL2 | | | | | | | | | |
| WNT5A)- | −0.192535039 | 0.12099156 | −0.0181433 | 0.04974618 | −0.1722268 | 0.06781625 | 0.13313975 | −0.1227189 | −0.1003338 |
| RYK | | | | | | | | | |
| WNT5A)- | −0.068584579 | −0.021508 | 0.08728452 | 0.0362349 | −0.0363909 | 0.13228834 | 0.17420667 | −0.1444692 | 0.42437495 |
| ROR2 | | | | | | | | | |
| WNT5A)- | −0.006928603 | −0.0457193 | −0.0069139 | 0.0220416 | −0.0123269 | 0.08307121 | 0.06538869 | 0.06750524 | −0.3356958 |
| PTPRK | | | | | | | | | |
| WNT5A)- | 0.029026051 | 0.01661155 | 0.27390653 | −0.0767974 | −0.0599403 | 0.0333751 | 0.1333684 | 0.12330923 | 0.4254699 |
| ADRB2 | | | | | | | | | |
| WNT5A)- | −0.004451941 | 0.04996066 | −0.1079908 | −0.1184925 | 0.07922675 | 0.06477228 | 0.07277156 | −0.0317295 | 0.27703218 |
| FZD1 | | | | | | | | | |
| WNT5A)- | 0.091801691 | −0.0660214 | −0.0506085 | 0.00357177 | 0.00385442 | 0.06774747 | −0.0352347 | 0.2160039 | 0.26821091 |
| FZD4 | | | | | | | | | |
| WNT5A)- | −0.018517134 | 0.03633807 | 0.0764807 | 0.05323094 | 0.01755214 | −0.024841 | 0.14274559 | 0.16565552 | 0.35466328 |
| FZD9 | | | | | | | | | |
| WNT5A)- | 0.050010117 | −0.0280336 | 0.0037054 | −0.0910552 | 0.011539 | 0.03045384 | 0.27339318 | −0.219576 | 0.42819417 |
| FZD7 | | | | | | | | | |
| WNT5A)- | −0.027595031 | −0.0405842 | 0.15543516 | 0.0356301 | −0.154481 | 0.1696687 | 0.17317624 | 0.16588455 | 0.44160704 |
| FZD3 | | | | | | | | | |
| WNT5A)- | −0.141719017 | 0.10708007 | 0.14253768 | −0.1045923 | −0.1416139 | 0.12716726 | 0.0965433 | −0.0054639 | 0.26348151 |
| FZD8 | | | | | | | | | |
| WNT5A)- | −0.0172193 | −0.0996107 | −0.0846314 | −0.3112801 | −0.2122759 | 0.393326 | 0.14372464 | −0.1879233 | 0.17348638 |
| ANTXR1 | | | | | | | | | |
| WNT5A)- | −0.048203417 | 0.06031779 | 0.11069317 | −0.0185621 | −0.0321404 | 0.11262271 | 0.20102835 | 0.04886359 | 0.12620743 |
| FZD6 | | | | | | | | | |
| ICAM1)- | −0.043036691 | −0.2446367 | 0.0633492 | 0.13236156 | −0.0729258 | 0.17291186 | −0.0672088 | 0.15320167 | 0.26017474 |
| ITGAM | | | | | | | | | |
| ICAM1)- | −0.179279717 | −0.1152233 | 0.12360924 | 0.19601545 | −0.1748078 | 0.20050592 | 0.06285977 | 0.04370154 | 0.04796513 |
| ITGAL | | | | | | | | | |
| ICAM1)- | −0.184917431 | −0.290584 | 0.23747116 | 0.06150236 | 0.04492942 | 0.11832255 | 0.10054432 | −0.0266838 | 0.06481171 |
| ITGAX | | | | | | | | | |
| ICAM1)- | −0.095345278 | 0.13415448 | −0.0537257 | −0.1667257 | 0.06077295 | 0.02464782 | −0.0031549 | −0.0115094 | −0.1358922 |
| MUC1 | | | | | | | | | |
| ICAM1)- | 0.032514286 | −0.1081301 | 0.00618417 | 0.03607005 | −0.0670658 | −0.106522 | 0.04895505 | −0.0958051 | 0.32080043 |
| EGFR | | | | | | | | | |
| ICAM1)- | −0.144401664 | 0.08051672 | 0.0498377 | 0.19441426 | 0.04820507 | −0.0254597 | −0.1051454 | −0.1362495 | 0.30384901 |
| CAV1 | | | | | | | | | |
| ICAM1)- | 0.080610471 | −0.0320924 | −0.0886854 | 0.05707021 | 0.05733008 | −0.0172096 | 0.06142909 | 0.06461895 | 0.18324211 |
| IL2RA | | | | | | | | | |
| ICAM1)- | −0.19578706 | −0.1742121 | 0.36176631 | 0.16519953 | −0.0072458 | 0.22252053 | −0.0435842 | −0.0121919 | 0.02202573 |
| ITGB2 | | | | | | | | | |
| ICAM1)- | −0.19047313 | −0.1130316 | 0.11018388 | 0.00543696 | 0.04247239 | 0.08599235 | 0.08756939 | 0.17581729 | 0.13955157 |
| SPN | | | | | | | | | |
| ICAM1)- | −0.039332884 | −0.2052129 | 0.18015007 | −0.114941 | −0.0113498 | 0.23168085 | 0.11333517 | 0.15919161 | 0.31782723 |
| IL2RG | | | | | | | | | |
| F13A1)- | −0.129113342 | −0.1784728 | 0.17988107 | 0.1269167 | −0.016579 | 0.10723875 | −0.0972661 | 0.2298858 | 0.37313512 |
| ITGA4 | | | | | | | | | |
| F13A1)- | −0.080107867 | 0.03041169 | 0.08072372 | −0.1259616 | −0.0136403 | 0.31372917 | 0.10149527 | −0.1200948 | 0.00830758 |
| ITGB1 | | | | | | | | | |
| BST1)- | −0.082764683 | −0.098576 | −0.0155325 | 0.05872476 | 0.01238837 | 0.00339217 | −0.0633992 | −0.0777369 | 0.1974219 |
| CAV1 | | | | | | | | | |
| KNG1)- | −0.044162363 | 0.00166891 | 0.178964 | 0.23243015 | −0.0807594 | 0.12953527 | −0.0199264 | 0.23330726 | 0.45582398 |
| ITGAM | | | | | | | | | |
| KNG1)- | 0.189571993 | −0.1223122 | 0.03854563 | −0.0282383 | −0.031517 | −0.0027395 | 0.08178727 | 0.22751826 | −0.0372701 |
| ADRA2A | | | | | | | | | |
| KNG1)- | −0.073819851 | −0.142957 | −0.0570844 | 0.06119071 | −0.0340897 | 0.1211485 | −0.0152129 | 0.19267008 | −0.0401466 |
| PLAUR | | | | | | | | | |
| KNG1)- | −0.110915429 | −0.0482208 | 0.01029387 | 0.16503179 | −0.1111808 | 0.12513862 | −0.2158843 | 0.26481845 | 0.30950512 |
| ITGB2 | | | | | | | | | |
| KNG1)- | 0.09094826 | −0.0308051 | −0.1123945 | 0.06445753 | −0.0913877 | −0.0855697 | −0.1061394 | −0.0750836 | 0.34732288 |
| SDC2 | | | | | | | | | |
| KNG1)- | 0.0384417 | 0.09721122 | 0.08842267 | 0.07600444 | −0.0474533 | 0.19098662 | −0.0104551 | 0.19008946 | 0.090489 |
| BDKRB2 | | | | | | | | | |
| KNG1)- | 0.127339025 | 0.07451908 | 0.1623282 | 0.17499392 | 0.08797623 | 0.14065213 | 0.11746578 | 0.26734584 | 0.46007886 |
| GP1BA | | | | | | | | | |
| VCAN)- | 0.126683657 | 0.2195969 | −0.0886687 | −0.1991124 | 0.01710285 | −0.0081074 | −0.1952412 | 0.18490201 | 0.33102551 |
| ITGA4 | | | | | | | | | |
| VCAN)- | 0.018987917 | 0.02159496 | −0.1014378 | −0.2500084 | 0.34688066 | −0.0191689 | −0.3324272 | 0.04009667 | 0.20552118 |
| CD44 | | | | | | | | | |
| VCAN)- | 0.082717363 | −0.2538034 | 0.16501043 | −0.0104774 | −0.0618817 | −0.1532805 | 0.2212014 | 0.08405624 | −0.3179742 |
| EGFR | | | | | | | | | |
| VCAN)- | −0.323292005 | 0.38303276 | 0.34916339 | −0.2108478 | −0.0482335 | 0.56766377 | 0.3776071 | −0.3868765 | 0.49702454 |
| ITGB1 | | | | | | | | | |
| VCAN)- | −0.050513839 | −0.0072501 | −0.0499168 | −0.1586976 | 0.14535144 | −0.1223792 | −0.1130587 | −0.016836 | 0.05993986 |
| TLR1 | | | | | | | | | |
| TNC)- | −0.075010988 | 0.18848726 | 0.15731292 | −0.2937125 | −0.2752347 | 0.37571755 | 0.06179017 | −0.1621945 | 0.40402138 |
| ITGA5 | | | | | | | | | |
| TNC)- | 0.04660498 | −0.1130787 | 0.01492229 | −0.1418067 | 0.01639544 | −0.015158 | 0.01496663 | −0.0852024 | −0.0044131 |
| SDC1 | | | | | | | | | |
| TNC)- | −0.160716143 | 0.00049222 | 0.16073577 | −0.1823597 | 0.00825176 | 0.13095636 | 0.02816437 | −0.1319073 | 0.18003339 |
| EGFR | | | | | | | | | |
| TNC)- | −0.055159115 | 0.07803349 | 0.00367699 | 0.05883551 | −0.0065693 | 0.17992276 | −0.0408935 | −0.0376564 | 0.17908856 |
| ITGB3 | | | | | | | | | |
| TNC)- | −0.064541081 | 0.12372956 | −0.0513103 | −0.1299213 | 0.09651716 | −0.0063803 | 0.23143566 | 0.11054982 | 0.06645771 |
| ITGB6 | | | | | | | | | |
| TNC)- | −0.026237089 | 0.12152339 | 0.14950998 | −0.0705998 | 0.01031513 | −0.0224919 | 0.0389889 | 0.07341503 | 0.14011131 |
| PTPRB | | | | | | | | | |
| TNC)- | −0.101070782 | 0.06742432 | 0.18837768 | 0.05788179 | 0.02683546 | −0.0686631 | 0.08625893 | 0.03451176 | 0.27354456 |
| ITGA8 | | | | | | | | | |
| TNC)- | −0.033022953 | 0.13024422 | 0.06933976 | 0.17735528 | 0.05895861 | 0.11406981 | −0.1678842 | 0.11613936 | 0.06334392 |
| ITGA7 | | | | | | | | | |
| TNC)- | −0.133683355 | 0.0337897 | 0.14459558 | −0.0668677 | 0.00136512 | 0.14509861 | 0.14187896 | −0.511571 | 0.38064592 |
| ITGB1 | | | | | | | | | |
| TNC)- | 0.077215497 | 0.02623661 | 0.0767438 | −0.0963146 | 0.13100794 | 0.09754776 | 0.31534776 | 0.28436136 | −0.0727704 |
| SDC4 | | | | | | | | | |
| TNC)- | −0.098707661 | −0.0306694 | 0.03973496 | 0.17041783 | 0.04573538 | 0.10516987 | 0.33358868 | −0.2277065 | 0.3259197 |
| CNTN1 | | | | | | | | | |
| TNC)- | −0.070631323 | 0.04485866 | 0.01917575 | −0.0185171 | 0.00408513 | 0.08030737 | −0.0128686 | −0.161932 | 0.02422266 |
| ITGA2 | | | | | | | | | |
| SEMA3A)- | 0.074875666 | 0.23329833 | 0.1128568 | −0.0442718 | −0.0564719 | 0.23699691 | 0.09083663 | 0.0887459 | 0.24119188 |
| PLXNA4 | | | | | | | | | |
| SEMA3A)- | −0.005075904 | −0.2231903 | 0.06633199 | −0.0647944 | 0.08680056 | −0.099699 | 0.04302819 | 0.01479521 | 0.0896187 |
| PLXNA3 | | | | | | | | | |
| SEMA3A)- | 0.022880147 | 0.043161 | −0.0480548 | 0.01064605 | 0.11579764 | 0.18007217 | 0.03634147 | 0.06366975 | 0.18296968 |
| PLXNA1 | | | | | | | | | |
| SEMA3A)- | 0.135810176 | 0.10527843 | 0.05453893 | 0.02376086 | −0.1730746 | 0.12719664 | −0.2270229 | 0.06788311 | 0.39577942 |
| NRP1 | | | | | | | | | |
| SEMA3A)- | 0.082213837 | −0.099835 | 0.11615467 | −0.091753 | 0.00024911 | 0.14943927 | 0.1329679 | 0.22345614 | −0.2233071 |
| PLXNA2 | | | | | | | | | |
| SEMA3A)- | −0.091678553 | 0.1329837 | 0.20788423 | 0.21938288 | 0.02812502 | 0.0647291 | −0.0525849 | −0.2139216 | 0.3539978 |
| NRP2 | | | | | | | | | |
| WNT2)- | −0.006916197 | 0.16278198 | 0.17761571 | −0.0722045 | 0.01522219 | 0.0538749 | 0.19974161 | −0.1410347 | 0.25814134 |
| FZD1 | | | | | | | | | |
| WNT2)- | 0.205803627 | 0.22426249 | 0.09361137 | 0.24639953 | 0.02184138 | 0.04587038 | 0.1552302 | 0.11950578 | 0.51006739 |
| FZD4 | | | | | | | | | |
| WNT2)- | −0.186706292 | 0.17988931 | 0.29929541 | 0.24295069 | −0.0160801 | 0.28527354 | 0.19313098 | 0.05636624 | 0.6049289 |
| FZD9 | | | | | | | | | |
| WNT2)- | −0.075414949 | 0.17371088 | 0.34761653 | 0.10015562 | −0.0554642 | 0.10369999 | 0.21645453 | −0.0936639 | 0.4201948 |
| FZD7 | | | | | | | | | |
| WNT2)- | −0.091518047 | 0.16043207 | 0.33339345 | 0.12648285 | −0.113769 | 0.3543122 | 0.27156357 | 0.16596098 | 0.34283128 |
| FZD3 | | | | | | | | | |
| WNT2)- | −0.039165956 | 0.08862382 | 0.2724193 | 0.10068619 | −0.0524472 | 0.14829937 | 0.06235726 | 0.11308378 | 0.18646677 |
| FZD8 | | | | | | | | | |
| WNT2)- | −0.007436578 | −0.0231474 | 0.18497363 | 0.01883613 | −0.1211168 | −0.0205801 | 0.12832617 | −0.0346785 | −0.0322394 |
| FZD5 | | | | | | | | | |
| NPTX2)- | 0.01560716 | −0.0219297 | 0.14819392 | 0.35374294 | 0.15099651 | 0.11056114 | 0.04420735 | 0.17215773 | 0.38546117 |
| NPTXR | | | | | | | | | |
| GAL)- | −0.010553274 | 0.01691449 | 0.04415096 | −0.0503559 | 0.03418313 | 0.0601986 | −0.0303815 | 0.11790502 | −0.2352486 |
| ADRA2A | | | | | | | | | |
| GAL)- | −0.03884464 | −0.0192484 | 0.23834172 | 0.15752109 | 0.14409885 | 0.18952488 | 0.17868697 | 0.08127004 | 0.16109826 |
| GALR2 | | | | | | | | | |
| ADAM28)- | 0.025579277 | 0.04318319 | −0.0252698 | 0.31047625 | −0.2531207 | 0.27760999 | 0.02795345 | 0.099722 | 0.13311481 |
| ITGA4 | | | | | | | | | |
| TIMP3)- | 0.184281216 | 0.14308563 | −0.0694131 | −0.1897709 | 0.28665131 | −0.0291708 | −0.2864672 | 0.16034566 | 0.26380296 |
| CD44 | | | | | | | | | |
| TIMP3)- | 0.255831223 | −0.0420009 | 0.16777981 | −0.034312 | −0.0860622 | −0.1379278 | 0.26989009 | 0.3320908 | 0.0469573 |
| MET | | | | | | | | | |
| TIMP3)- | −0.106405055 | −0.1840786 | −0.0397432 | −0.2468528 | −0.0358162 | −0.025286 | −0.0434028 | −0.0432395 | −0.0660913 |
| AGTR2 | | | | | | | | | |
| LUM)- | −0.290207911 | 0.26063675 | 0.31558165 | −0.4220603 | −0.2169354 | 0.64360957 | 0.34173463 | −0.3148701 | 0.30448875 |
| ITGB1 | | | | | | | | | |
| FGF7)- | −0.107711395 | 0.08168322 | 0.16107035 | −0.3478693 | −0.0883186 | 0.05330079 | 0.15334616 | −0.0406576 | 0.16369974 |
| FGFR2 | | | | | | | | | |
| FGF7)- | −0.040208676 | 0.08934451 | 0.28412941 | −0.0447858 | −0.1073536 | 0.16956831 | −0.0487614 | −0.070823 | −0.1170217 |
| FGFR4 | | | | | | | | | |
| FGF7)- | 0.044166799 | 0.04650312 | −0.0002418 | −0.2126921 | −0.136738 | 0.21258913 | −0.1191879 | −0.0090879 | 0.10145133 |
| FGFR1 | | | | | | | | | |
| FGF7)- | 0.036693461 | 0.0510803 | 0.05378747 | −0.1843425 | −0.093355 | 0.14512221 | 0.03793279 | 0.09238763 | −0.0257635 |
| FGFR3 | | | | | | | | | |
| MFGE8)- | −0.01839049 | 0.001743 | −0.0021134 | −0.0889203 | 0.00031409 | −0.0747669 | 0.04953766 | −0.0383832 | 0.0945709 |
| ITGB3 | | | | | | | | | |
| IGFBP4)- | 0.026200336 | −0.1913028 | −0.056427 | 0.01472318 | 0.09295062 | −0.0300258 | −0.1619796 | 0.00662991 | −0.1503066 |
| FZD8 | | | | | | | | | |
| CGN)- | 0.486829771 | −0.3459763 | 0.436381 | −0.3444023 | 0.38472174 | 0.56534475 | −0.3192681 | −0.3042084 | 0.6382199 |
| F11R | | | | | | | | | |
| CGN)- | 0.069627243 | −0.1508073 | 0.0667486 | −0.159479 | 0.00016991 | 0.12484584 | 0.22498198 | 0.05457512 | 0.0803077 |
| TGFBR2 | | | | | | | | | |
| CGN)- | 0.2229406 | −0.2327451 | 0.26162497 | −0.2120271 | 0.25011388 | 0.3302676 | −0.133804 | −0.2678158 | 0.4404916 |
| OCLN | | | | | | | | | |
| LEFTY1)- | 0.103267275 | −0.225569 | −0.0398733 | −0.0861603 | 0.02672189 | −0.0753513 | 0.0532622 | −0.0635378 | −0.1737007 |
| ACVR1B | | | | | | | | | |
| REN)- | −0.030108603 | −0.0755059 | 0.06701415 | −0.163178 | −0.0974239 | 0.0079928 | −0.0260162 | −0.0790628 | −0.2353512 |
| ATP6AP2 | | | | | | | | | |
| CALM2)- | −0.17400654 | 0.06401767 | 0.05847734 | −0.0615484 | 0.05926426 | −0.1055574 | 0.08182931 | 0.0256583 | 0.15905113 |
| KCNQ3 | | | | | | | | | |
| CALM2)- | 0.047393948 | −0.114228 | 0.07032963 | −0.1649315 | −0.0146176 | 0.00866758 | 0.07492482 | 0.02407344 | 0.27438556 |
| EGFR | | | | | | | | | |
| CALM2)- | 0.183107424 | 0.01117538 | 0.04607243 | −0.1275579 | −0.0521129 | 0.39077935 | −0.0824195 | 0.01711583 | −0.0156631 |
| MYLK | | | | | | | | | |
| CALM2)- | 0.026308581 | −0.0008378 | 0.13219649 | 0.00478653 | 0.03394272 | −0.1190636 | 0.02680783 | 0.00948978 | −0.0579889 |
| INSR | | | | | | | | | |
| CALM2)- | 0.062996233 | −0.0666929 | −0.0885276 | −0.0839105 | −0.0765828 | 0.08090039 | 0.15461003 | 0.04768664 | 0.07393433 |
| GP6 | | | | | | | | | |
| CALM2)- | −0.027434108 | 0.06929623 | −0.2409605 | 0.05013563 | −0.1820428 | −0.1351536 | 0.01553194 | 0.03241813 | 0.00335408 |
| SCN10A | | | | | | | | | |
| CALM2)- | −0.077754861 | 0.04949403 | −0.0388239 | −0.0119136 | −0.0925124 | −0.0359149 | 0.07569457 | −0.2169643 | −0.0808057 |
| PLPP6 | | | | | | | | | |
| CALM2)- | −0.139684234 | 0.03562065 | 0.09194297 | −0.0700961 | 0.04155433 | −0.1769232 | −0.0192004 | 0.17546078 | 0.01677041 |
| AQP6 | | | | | | | | | |
| CALM2)- | −0.026372107 | −0.0772699 | 0.08790362 | −0.1960899 | −0.0326794 | −0.0206668 | −0.0899694 | 0.15614678 | 0.13421018 |
| AQP1 | | | | | | | | | |
| CALM2)- | 0.096356883 | 0.03720547 | 0.01542777 | −0.0805745 | 0.00109078 | 0.03621889 | 0.04169295 | 0.19257168 | 0.03233692 |
| SCN4A | | | | | | | | | |
| NXPH2)- | 0.025848654 | 0.18226331 | 0.28331144 | 0.14939158 | −0.2337212 | 0.25120969 | −0.0155888 | 0.1864447 | 0.33221335 |
| NRXN1 | | | | | | | | | |
| LRIG1)- | 0.147577996 | 0.00727792 | 0.07237775 | 0.0477948 | 0.04913941 | −0.038067 | 0.13530363 | 0.19717225 | −0.0591293 |
| EGFR | | | | | | | | | |
| LRIG1)- | 0.054695226 | 0.01353901 | 0.08591846 | 0.17528929 | 0.01827397 | −0.1069258 | 0.18771677 | −0.0047309 | −0.0946352 |
| MET | | | | | | | | | |
| UCN2)- | −0.051140053 | −0.1084322 | 0.07873255 | −0.0019135 | 0.03481896 | 0.16952976 | 0.06021809 | 0.14783472 | 0.0802081 |
| IL10RB | | | | | | | | | |
| UCN2)- | 0.193918843 | 0.17236975 | 0.12831886 | 0.19447416 | 0.16533182 | 0.1342136 | 0.14964773 | 0.11128427 | 0.37196522 |
| CRHR2 | | | | | | | | | |
| UCN2)- | 0.110966553 | 0.14342409 | 0.19233363 | 0.28424143 | 0.07759874 | 0.09289735 | 0.20540749 | 0.22978949 | 0.33062175 |
| CRHR1 | | | | | | | | | |
| SFRP2)- | 0.252225624 | −0.044301 | 0.11203241 | 0.10199302 | −0.1125431 | −0.2141803 | 0.22663977 | 0.33687695 | −0.254267 |
| FZD5 | | | | | | | | | |
| MEGF10)- | −0.010639296 | 0.07068675 | 0.06402972 | 0.12960059 | −0.0389696 | 0.0261251 | 0.09939807 | −0.14088 | 0.20025837 |
| ABCA1 | | | | | | | | | |
| IL9)- | 0.180072317 | 0.21084578 | 0.11380131 | 0.14560846 | −0.0860873 | 0.20322483 | 0.07152905 | 0.20963764 | 0.29211638 |
| IL2RG | | | | | | | | | |
| FGF18)- | 0.056709839 | 0.08725999 | 0.15974169 | 0.05885937 | −0.0176323 | 0.1913214 | 0.03030379 | 0.11902523 | 0.3089535 |
| FGFR2 | | | | | | | | | |
| FGF18)- | −0.005706069 | 0.01035671 | 0.02868116 | −0.0719993 | −0.1376451 | 0.03800984 | −0.0690271 | 0.00231519 | 0.00053607 |
| FGFR4 | | | | | | | | | |
| FGF18)- | −0.013565412 | 0.07572386 | −0.0018841 | 0.0199889 | 0.00918899 | −0.1372257 | −0.0586609 | −0.1247775 | 0.32661102 |
| FGFR1 | | | | | | | | | |
| FGF18)- | −0.079419945 | 0.06357125 | 0.06524839 | 0.04682595 | −0.1237774 | 0.06487051 | −0.00086 | −0.0005361 | 0.20938295 |
| FGFR3 | | | | | | | | | |
| CRH)- | 0.015527337 | 0.34664382 | 0.04844619 | 0.15882046 | 0.01163597 | 0.27846219 | 0.13037663 | 0.23547139 | 0.4261993 |
| ADRB2 | | | | | | | | | |
| CRH)- | 0.04038327 | 0.27500531 | 0.2708397 | 0.111116 | 0.07926514 | 0.17077864 | 0.24183006 | 0.19558044 | 0.39636412 |
| CRHR2 | | | | | | | | | |
| CRH)- | 0.041831791 | 0.30330425 | 0.0258358 | 0.10736521 | 0.10681328 | 0.23963677 | 0.26822987 | 0.11510263 | 0.3876851 |
| GPR84 | | | | | | | | | |
| CRH)- | 0.105174981 | 0.14071683 | 0.12196223 | 0.14379322 | 0.07905585 | 0.27032818 | 0.13254636 | 0.26628726 | 0.4262491 |
| PTH1R | | | | | | | | | |
| CRH)- | 0.018584741 | 0.00843137 | −0.1002656 | 0.05875829 | 0.08015816 | 0.16547034 | 0.1788646 | 0.1655853 | 0.23669055 |
| VIPR1 | | | | | | | | | |
| CRH)- | 0.004265463 | 0.11375577 | 0.04799802 | 0.08707196 | 0.07286901 | 0.25537728 | 0.2695377 | 0.09157747 | 0.3363398 |
| MC2R | | | | | | | | | |
| CRH)- | 0.074594007 | 0.14532443 | −0.0846222 | 0.05581018 | 0.01692341 | 0.24422533 | 0.0993158 | 0.26660085 | 0.40512839 |
| CRHR1 | | | | | | | | | |
| CRH)- | −0.064134648 | 0.31032308 | 0.16849258 | 0.21097623 | 0.07142072 | 0.28056686 | 0.2586792 | 0.03998301 | 0.45087301 |
| ADRB3 | | | | | | | | | |
| LIN7C)- | −0.063251345 | −0.0060794 | 0.16264582 | 0.11203045 | 0.04318037 | 0.00036179 | 0.07569447 | 0.06582465 | −0.0126196 |
| ABCA1 | | | | | | | | | |
| SERPING1)- | 0.079682077 | −0.0081964 | −0.0468635 | 0.05617976 | 0.10535106 | −0.1752348 | 0.05425821 | 0.07075422 | 0.19297254 |
| SELE | | | | | | | | | |
| IL18)- | 0.021272543 | 0.09222989 | 0.05628268 | 0.23038655 | −0.0594068 | 0.12482876 | 0.0629078 | 0.09213637 | 0.11766326 |
| CD48 | | | | | | | | | |
| IL18)- | 0.011929811 | 0.08543428 | −0.004011 | 0.14929558 | 0.01452197 | 0.14983343 | 0.05523043 | 0.05677249 | 0.09359264 |
| IL18R1 | | | | | | | | | |
| IL18)- | 0.028892875 | 0.05054263 | 0.09722821 | 0.1163636 | −0.0903558 | 0.34159002 | 0.04131793 | 0.0020976 | −0.0226233 |
| IL18BP | | | | | | | | | |
| QDPR)- | 0.092764312 | 0.14097743 | 0.11501294 | 0.13592755 | 0.25541256 | 0.0286112 | 0.17777057 | 0.05421874 | 0.28851472 |
| DYSF | | | | | | | | | |
| PIGF)- | −0.11833586 | −0.0219445 | 0.05077206 | 0.03973227 | −0.0490184 | 0.0859852 | −0.0899096 | 0.03867458 | −0.0295223 |
| FLT1 | | | | | | | | | |
| PTH)- | 0.108562604 | 0.27522546 | 0.07087547 | 0.12071869 | 0.05997514 | 0.15089269 | 0.09393617 | 0.09191021 | 0.61457545 |
| ADRB2 | | | | | | | | | |
| PTH)- | 0.167552295 | 0.21145995 | 0.07585322 | 0.15542953 | 0.0168964 | 0.17001467 | −0.0067886 | 0.16646721 | 0.48309923 |
| GPR84 | | | | | | | | | |
| PTH)- | −0.084165021 | 0.26302558 | 0.17032961 | 0.21414476 | −0.0065493 | 0.32675374 | 0.27614729 | 0.22242634 | 0.48014869 |
| PTH1R | | | | | | | | | |
| PTH)- | −0.089217045 | 0.06211766 | 0.1820313 | −0.0173019 | −0.1319726 | 0.35332995 | 0.00020077 | 0.16561606 | 0.19935613 |
| VIPR1 | | | | | | | | | |
| PTH)- | 0.086453013 | 0.13253116 | 0.21900517 | 0.31213913 | −0.0241525 | 0.1037141 | 0.20168135 | 0.06013714 | 0.54214787 |
| ADRB3 | | | | | | | | | |
| BMP3)- | −0.121698859 | −0.0567055 | 0.06436731 | −0.0845319 | −0.1085696 | 0.07306104 | −0.1312914 | −0.0584754 | −0.0515959 |
| BMPR2 | | | | | | | | | |
| BMP3)- | 0.184550822 | 0.22588698 | 0.17605487 | 0.25042992 | 0.01135074 | 0.16557053 | 0.18552222 | 0.09686087 | 0.28951782 |
| BMPR1A | | | | | | | | | |
| BMP3)- | −0.028214927 | 0.13641178 | 0.18356107 | 0.20990293 | 0.04024772 | 0.2859758 | 0.17604162 | 0.03379767 | 0.41291198 |
| BMPR1B | | | | | | | | | |
| BMP6)- | −0.038538135 | −0.2217213 | −0.1053728 | −0.1531878 | −0.1540529 | −0.026309 | −0.2206058 | 0.04437791 | −0.1687938 |
| BMPR2 | | | | | | | | | |
| BMP6)- | −0.005954483 | 0.14265537 | 0.25673719 | 0.27295126 | −0.0493223 | 0.2463735 | 0.11725757 | 0.06612268 | 0.47701138 |
| BMPR1A | | | | | | | | | |
| BMP6)- | −0.137038976 | 0.25603352 | 0.15946514 | 0.2430645 | −0.0467896 | 0.30818745 | 0.23237994 | 0.13642706 | 0.53192686 |
| BMPR1B | | | | | | | | | |
| SEMA3D)- | 0.099476509 | 0.17320395 | −0.1049082 | 0.06150492 | 0.01969188 | 0.00824734 | −0.2954505 | 0.28339375 | 0.42712915 |
| PLXND1 | | | | | | | | | |
| APP)- | 0.162696652 | 0.00020945 | 0.25536995 | −0.013702 | 0.19249788 | 0.12596448 | −0.0083985 | 0.01537605 | 0.48925668 |
| LRP10 | | | | | | | | | |
| APP)- PLD1 | 0.048098529 | −0.1564533 | 0.08722291 | −0.0873482 | 0.05565268 | −0.0459449 | −0.0600852 | −0.0080827 | 0.07114264 |
| APP)- | 0.036218553 | 0.0816166 | −0.0103524 | 0.07081989 | −0.0633382 | −0.0573329 | 0.02121424 | 0.04047156 | 0.26692425 |
| TSPAN15 | | | | | | | | | |
| APP)- | −0.000566872 | 0.03700064 | 0.20662328 | −0.2040395 | −0.071954 | 0.16958572 | 0.18708874 | 0.19181457 | −0.0402137 |
| CAV1 | | | | | | | | | |
| APP)- | 0.056562928 | 0.28251333 | −0.0610745 | 0.10273483 | 0.15244637 | 0.13526494 | 0.04971681 | 0.10497238 | 0.07316001 |
| RPSA | | | | | | | | | |
| APP)- | 0.220716525 | 0.05397037 | 0.14913057 | 0.19335335 | −0.1047823 | −0.0062893 | 0.0757758 | 0.08803554 | −0.0550419 |
| TNFRSF21 | | | | | | | | | |
| APP)- | 0.123728796 | −0.2542886 | 0.22791856 | −0.1043113 | 0.04213787 | 0.03833467 | −0.0526187 | −0.0370515 | 0.03649375 |
| NCSTN | | | | | | | | | |
| APP)- | 0.101100355 | 0.07539913 | −0.1954972 | 0.07295843 | −0.2275169 | 0.08717207 | −0.0177743 | −0.122841 | −0.032173 |
| AGER | | | | | | | | | |
| WNT7A)- | 0.113384698 | −0.1886436 | 0.16244657 | −0.0686811 | −0.0138197 | 0.023087 | −0.086304 | −0.0097752 | 0.17428978 |
| LDLR | | | | | | | | | |
| WNT7A)- | −0.090028715 | 0.33414553 | 0.24211245 | 0.18808324 | 0.07747954 | −0.1041256 | 0.00079752 | −0.0482264 | 0.04893767 |
| RECK | | | | | | | | | |
| WNT7A)- | −0.090048671 | 0.17397898 | 0.27206043 | 0.14074856 | −0.0080995 | 0.17540845 | 0.05949671 | 0.01307882 | −0.0794026 |
| FZD9 | | | | | | | | | |
| WNT7A)- | 0.020153699 | −0.0812701 | −0.0937867 | −0.1688931 | 0.01796391 | 0.16949441 | 0.09365502 | 0.02489132 | −0.2852167 |
| FZD5 | | | | | | | | | |
| DKK2)- | 0.011242129 | 0.11861544 | 0.19495595 | 0.19863646 | 0.11381724 | 0.05633594 | 0.18558765 | −0.0535249 | 0.37678565 |
| KREMEN2 | | | | | | | | | |
| CXCL13)- | −0.078605057 | 0.11991415 | 0.40174335 | 0.12857605 | −0.1680342 | 0.15321502 | 0.03111423 | 0.17400601 | 0.31260536 |
| CXCR5 | | | | | | | | | |
| GDF6)- | 0.123098612 | −0.1669384 | −0.1649349 | −0.0789895 | −0.0733937 | −0.0824293 | −0.2627607 | −0.0555515 | −0.2012098 |
| BMPR2 | | | | | | | | | |
| GDF6)- | 0.020950609 | 0.12486518 | 0.32327052 | 0.15461911 | 0.06310406 | 0.07640365 | 0.12616547 | −0.0031995 | 0.29684321 |
| BMPR1A | | | | | | | | | |
| GDF6)- | −0.099167319 | 0.25589423 | 0.29380291 | 0.11269126 | −0.0288933 | 0.31057704 | 0.29822703 | 0.15324965 | 0.47684944 |
| BMPR1B | | | | | | | | | |
| SST)- | −0.002048577 | −0.0400648 | 0.05798015 | 0.1548231 | −0.0090537 | 0.13851493 | 0.13369531 | 0.0477723 | 0.16696619 |
| SSTR5 | | | | | | | | | |
| WNT4)- | −0.136119003 | −0.0047265 | 0.14794185 | 0.00513075 | −0.1240664 | 0.19692028 | −0.0088836 | 0.10341519 | 0.13869641 |
| FZD8 | | | | | | | | | |
| WNT4)- | −0.001629908 | −0.0684869 | 0.0654612 | 0.24508096 | 0.00509341 | −0.0321623 | 0.1219598 | −0.0227768 | 0.08610134 |
| FZD6 | | | | | | | | | |
| ACE)- | 0.207346146 | 0.09445072 | 0.22564832 | 0.02789622 | −0.0942081 | 0.10590381 | −0.0545835 | −0.221104 | −0.1458229 |
| BDKRB2 | | | | | | | | | |
| ACE)- | −0.017235996 | 0.21880865 | 0.207675 | 0.10514845 | 0.07731843 | 0.17634579 | −0.0199655 | 0.08027604 | 0.22091313 |
| AGTR2 | | | | | | | | | |
| CALM3)- | −0.122475379 | 0.12664198 | 0.09033169 | 0.15209163 | 0.07548431 | −0.0450768 | −0.1789954 | 0.08198628 | −0.1703322 |
| KCNQ3 | | | | | | | | | |
| CALM3)- | −0.048216785 | −0.0183386 | 0.03539387 | 0.13945354 | 0.09783751 | 0.02670047 | −0.2673579 | 0.08254012 | −0.1737971 |
| ESR1 | | | | | | | | | |
| CALM3)- | 0.049233379 | 0.05215654 | 0.09407746 | 0.13334857 | 0.13889987 | −0.0178256 | −0.0777063 | 0.20076116 | −0.2281479 |
| MYLK | | | | | | | | | |
| CALM3)- | −0.061574118 | 0.11033024 | 0.05503652 | 0.23164919 | −0.0876074 | −0.0216232 | 0.25938246 | 0.09386225 | −0.1514317 |
| INSR | | | | | | | | | |
| CALM3)- | −0.049472098 | 0.15851086 | 0.0777005 | 0.17510165 | 0.16670723 | 0.11079598 | 0.11108866 | 0.0110248 | −0.1684668 |
| GP6 | | | | | | | | | |
| CALM3)- | −0.027866336 | 0.07454084 | 0.04339594 | 0.15823741 | 0.11123147 | −0.0238136 | −0.1918046 | −0.0504293 | −0.2057389 |
| SCN10A | | | | | | | | | |
| CALM3)- | −0.107408694 | 0.18797064 | 0.17818954 | 0.20117735 | 0.16425438 | −0.1174203 | −0.1411567 | −0.0918827 | −0.3051868 |
| AQP6 | | | | | | | | | |
| CALM3)- | 0.003616666 | −0.0020829 | 0.05490316 | 0.04240672 | 0.11749062 | 0.02503219 | −0.1597761 | 0.03370689 | −0.1331234 |
| AR | | | | | | | | | |
| CALM3)- | −0.020007207 | 0.10857857 | 0.1595356 | 0.23871245 | 0.04380277 | 0.06646042 | 0.15289316 | 0.01917268 | −0.0339671 |
| AQP1 | | | | | | | | | |
| CALM3)- | −0.007829494 | 0.15793336 | −0.0194694 | 0.07844211 | 0.18502529 | −0.0585396 | −0.1044026 | 0.05746648 | −0.2342006 |
| SCN4A | | | | | | | | | |
| TFF2)- | 0.00700235 | −0.0968652 | 0.1701054 | −0.0055776 | −0.0621872 | 0.38515286 | −0.0477393 | −0.1063457 | 0.04036904 |
| MUC6 | | | | | | | | | |
| TFF1)- | 0.492224048 | −0.5710515 | 0.48818515 | −0.6409765 | 0.52782111 | 0.55923497 | −0.1913956 | −0.1859371 | 0.22653028 |
| MUC5AC | | | | | | | | | |
| S100B)- | 0.123288432 | 0.1535261 | 0.15144669 | 0.07775678 | 0.05770017 | 0.02348117 | −0.0604211 | −0.018933 | 0.18132259 |
| ALCAM | | | | | | | | | |
| S100B)- | −0.050427727 | 0.15069348 | 0.07663607 | 0.08516473 | 0.08217934 | 0.19224481 | 0.19933936 | 0.06121833 | 0.3872474 |
| AGER | | | | | | | | | |
| S100A1)- | 0.096530978 | 0.13585352 | 0.21207064 | 0.05138339 | 0.08976214 | 0.13951499 | 0.13105973 | 0.26945649 | 0.33076822 |
| TRPM3 | | | | | | | | | |
| S100A1)- | 0.091834434 | 0.13487478 | 0.21735359 | 0.09928619 | −0.047662 | 0.05175938 | 0.11776512 | 0.20987979 | 0.42636042 |
| RYR1 | | | | | | | | | |
| S100A1)- | 0.040892264 | 0.03288083 | 0.1927891 | 0.00791449 | 0.06206791 | 0.19909968 | 0.13701278 | −0.1265896 | 0.38311241 |
| TLR4 | | | | | | | | | |
| S100A1)- | 0.121108625 | 0.19132227 | 0.02582996 | −0.0359603 | 0.125022 | 0.13294546 | 0.14637116 | −0.0411423 | 0.40022439 |
| AGER | | | | | | | | | |
| SCGB3A1)- | −0.09067566 | 0.05607431 | 0.08699472 | 0.12524646 | −0.056897 | 0.09680562 | 0.01209172 | 0.09234722 | 0.23825189 |
| MARCO | | | | | | | | | |
| CXCL16)- | 0.086207453 | −0.0113873 | 0.00085152 | 0.00535496 | 0.08435836 | −0.0062728 | −0.0299239 | −0.1244783 | −0.1094843 |
| CXCR6 | | | | | | | | | |
| TNFSF12)- | −0.072707753 | 0.1162292 | 0.18023845 | −0.0923616 | −0.0413736 | 0.04776184 | −0.0377268 | 0.07946389 | 0.03163676 |
| TNFRSF25 | | | | | | | | | |
| TNFSF12)- | 0.090237006 | −0.0696202 | 0.11944145 | −0.2109869 | 0.01758179 | 0.03999508 | −0.1464898 | −0.1412462 | −0.2930686 |
| TNFRSF12A | | | | | | | | | |
| FGF11)- | 0.035605598 | 0.22734443 | 0.03393355 | 0.13189622 | −0.1729172 | 0.22897712 | 0.06104139 | 0.08764433 | 0.27099564 |
| FGFR2 | | | | | | | | | |
| FGF11)- | −0.091351825 | 0.20696256 | −0.0234529 | −0.0084164 | −0.1298737 | 0.00812879 | −0.1113022 | −0.0950148 | 0.26784328 |
| FGFR1 | | | | | | | | | |
| FGF11)- | 0.003423198 | 0.15218302 | −0.0117639 | 0.09567824 | 0.0636747 | 0.03032075 | 0.05490226 | 0.11870328 | 0.23012444 |
| FGFR3 | | | | | | | | | |
| FGF19)- | 0.02783592 | 0.24945051 | 0.17128214 | −0.0672895 | 0.11358463 | 0.05994487 | −0.0201743 | 0.16274256 | 0.27771341 |
| FGFR2 | | | | | | | | | |
| FGF19)- | −0.104211587 | 0.03157875 | −0.0252196 | 0.02765421 | 0.09687965 | −0.1851975 | −0.1647523 | 0.04957226 | 0.23085531 |
| FGFR1 | | | | | | | | | |
| FGF19)- | 0.006844016 | 0.07099891 | 0.04352874 | −0.1473382 | 0.0265422 | 0.08555494 | −0.0208844 | 0.00774893 | 0.24378612 |
| FGFR3 | | | | | | | | | |
| VCAM1)- | −0.060375862 | −0.0257548 | 0.10230812 | 0.01171023 | −0.0342781 | −0.0220643 | −0.0607989 | 0.04558983 | 0.23231603 |
| ITGA4 | | | | | | | | | |
| VCAM1)- | −0.177031299 | 0.16243598 | 0.1561637 | −0.2919618 | −0.1332597 | 0.28338888 | −0.2100851 | −0.1293836 | −0.2912336 |
| ITGB1 | | | | | | | | | |
| VCAM1)- | 0.085911407 | 0.1210895 | −0.1161242 | 0.02242138 | 0.0606624 | 0.00478722 | −0.1163555 | 0.03071984 | 0.08001148 |
| ITGB2 | | | | | | | | | |
| VCAM1)- | 0.142872637 | −0.1374428 | 0.15819725 | 0.09313091 | −0.107891 | −0.1935396 | 0.10816226 | 0.08450519 | −0.3154528 |
| EZR | | | | | | | | | |
| VCAM1)- | −0.0090001 | 0.05266119 | −0.0347805 | 0.08422752 | 0.12903032 | 0.06379578 | −0.0389215 | 0.11164311 | 0.15707337 |
| MSN | | | | | | | | | |
| ARPC5)- | −0.079151279 | 0.02817516 | 0.03685628 | 0.08536653 | 0.13349314 | −0.1921389 | 0.06152661 | −0.2374373 | −0.3976964 |
| ADRB2 | | | | | | | | | |
| ARPC5)- | −0.074555313 | 0.20339169 | −0.0280448 | 0.14760204 | 0.17459125 | 0.13929863 | −0.2860844 | −0.1882281 | 0.29866202 |
| LDLR | | | | | | | | | |
| INHBB)- | 0.299905382 | −0.1179204 | 0.28002953 | −0.1413504 | 0.26823117 | 0.37616332 | 0.17945041 | 0.10830723 | 0.08369597 |
| SMAD3 | | | | | | | | | |
| INHBB)- | −0.100965862 | −0.2208855 | 0.06297716 | −0.2014626 | −0.0086393 | 0.08993277 | −0.0331906 | −0.0156875 | −0.3909826 |
| ACVR1B | | | | | | | | | |
| PROK2)- | 0.202944688 | 0.16554246 | 0.13025518 | 0.21781961 | 0.09289386 | 0.21408331 | 0.13459573 | 0.21359546 | 0.5803321 |
| PROKR1 | | | | | | | | | |
| IHH)- | 0.093262413 | 0.07815607 | 0.04911914 | 0.17455383 | 0.07170131 | 0.05636198 | −0.087102 | 0.34211398 | −0.1265649 |
| PTCH1 | | | | | | | | | |
| IHH)- HHIP | −0.255479691 | 0.18897477 | 0.28652737 | 0.08020743 | −0.0959397 | 0.11841516 | 0.05077416 | 0.17622966 | −0.0852176 |
| IHH)- BOC | −0.167409972 | 0.22402273 | 0.19090852 | 0.06736013 | 0.05747056 | 0.05328211 | 0.10942474 | −0.0382104 | 0.06156487 |
| IHH)- | 0.001748623 | 0.24509654 | 0.28920138 | 0.01014565 | 0.02350953 | 0.17223956 | −0.0002667 | −0.0216514 | 0.24362957 |
| PTCH2 | | | | | | | | | |
| CXCL3)- | −0.036972636 | −0.0132885 | −0.075098 | 0.03020661 | −0.0243943 | 0.0352077 | 0.10255631 | −0.1258461 | −0.3252107 |
| CXCR2 | | | | | | | | | |
| CXCL5)- | −0.021213391 | 0.18383973 | 0.06013507 | 0.19890176 | 0.02111624 | 0.00280807 | 0.1610375 | −0.1250278 | −0.2382893 |
| CXCR2 | | | | | | | | | |
| PPBP)- | 0.081558101 | 0.13983423 | 0.20215971 | 0.0922873 | −0.1022173 | 0.12382323 | 0.10589014 | 0.05780757 | 0.40745661 |
| CXCR2 | | | | | | | | | |
| PF4)- | 0.091201031 | 0.25243062 | 0.09605205 | 0.12911027 | 0.03300478 | 0.14369356 | −0.0022126 | −0.0100531 | 0.26388869 |
| FGFR2 | | | | | | | | | |
| PF4)- SDC2 | 0.024107422 | −0.1771598 | −0.1719242 | 0.22104028 | 0.13894659 | −0.0587704 | −0.2575989 | 0.02307865 | 0.10802181 |
| PF4)- LDLR | −0.117008708 | 0.08780795 | −0.0519002 | −0.1511794 | 0.09130193 | 0.0603914 | 0.24193043 | 0.14207543 | −0.2434833 |
| PF4)- | 0.074629648 | 0.11205479 | −0.0710004 | 0.02209387 | 0.00176879 | 0.16265173 | 0.04248186 | 0.0537742 | 0.09157991 |
| THBD | | | | | | | | | |
| UCN)- | 0.012256256 | 0.1880469 | 0.08762278 | 0.2168264 | 0.05862255 | 0.20510217 | 0.25793172 | 0.09259137 | 0.41612303 |
| CRHR1 | | | | | | | | | |
| UCN)- | 0.084526033 | 0.30229071 | 0.22480738 | 0.20363372 | 0.05292057 | 0.15920738 | 0.25983537 | 0.18715525 | 0.39906145 |
| CRHR2 | | | | | | | | | |
| TDGF1)- | 0.027137483 | 0.0809182 | 0.08143318 | 0.035988 | 0.00771239 | 0.06558837 | 0.1243537 | 0.1487644 | 0.05551357 |
| SMAD3 | | | | | | | | | |
| TDGF1)- | −0.008543027 | −0.1645044 | 0.20087078 | −0.1480951 | 0.16110291 | 0.03154344 | 0.0374194 | −0.0752269 | −0.4489172 |
| ACVR1B | | | | | | | | | |
| LIPH)- | 0.173849925 | 0.21919 | −0.0147639 | 0.32693942 | −0.1757609 | 0.19684451 | 0.11166628 | −0.2663174 | −0.322675 |
| LPAR1 | | | | | | | | | |
| LIPH)- | 0.015248435 | 0.04827335 | 0.33893625 | 0.1770638 | 0.23718403 | 0.10675086 | −0.4283952 | −0.1160113 | −0.2853393 |
| LPAR2 | | | | | | | | | |
| MELTF)- | 0.183819383 | 0.10399274 | 0.0920013 | 0.03935859 | 0.11035398 | 0.13110509 | −0.0198054 | 0.07603023 | −0.2295518 |
| TFRC | | | | | | | | | |
| SPINK1)- | 0.19945528 | 0.14932115 | 0.0025444 | 0.25328351 | 0.05852623 | 0.17099074 | −0.0723183 | −0.2104461 | −0.3047271 |
| NRSN1 | | | | | | | | | |
| IL3)- | −0.03015512 | 0.13240664 | 0.05255589 | 0.18317463 | −0.0904391 | 0.16429637 | 0.07014521 | 0.1371031 | 0.49283853 |
| CSF2RB | | | | | | | | | |
| IL3)- | 0.020011136 | 0.22521911 | 0.14472385 | 0.09409052 | 0.03239141 | 0.1843878 | −0.0331258 | 0.16188204 | 0.5593593 |
| IL3RA | | | | | | | | | |
| CSF2)- | 0.00755997 | −0.2455518 | −0.1950593 | −0.0564058 | −0.0048108 | −0.0721691 | −0.075823 | −0.1593284 | −0.1632816 |
| ITGB1 | | | | | | | | | |
| CSF2)- | −0.134995135 | 0.23723558 | 0.2108701 | 0.09859147 | 0.05482169 | 0.17227131 | −0.0450252 | 0.14170089 | 0.41737423 |
| CSF2RB | | | | | | | | | |
| CSF2)- | 0.068792987 | 0.12362597 | −0.0847721 | −0.0066083 | 0.10112874 | 0.06743869 | −0.0779862 | 0.19220145 | 0.38269813 |
| CSF2RA | | | | | | | | | |
| CSF2)- | 0.111025467 | 0.141358 | 0.23478887 | 0.12421058 | −0.0298852 | 0.06082648 | 0.11327077 | 0.17034201 | 0.37136413 |
| IL3RA | | | | | | | | | |
| CSF2)- | 0.05846012 | −0.0019669 | −0.1081403 | 0.06863788 | 0.06921739 | −0.0511131 | −0.0795694 | −0.0718113 | 0.19904855 |
| SDC2 | | | | | | | | | |
| CSF2)- | 0.05659087 | 0.05875259 | −0.0617747 | 0.19185909 | −0.0941531 | 0.16587434 | −0.0371857 | 0.14191803 | 0.32128399 |
| CSF3R | | | | | | | | | |
| SHH)- | −0.175184621 | 0.14419543 | 0.20658704 | 0.15380919 | 0.04453079 | 0.09103299 | −0.2868055 | 0.14168549 | 0.11136987 |
| SCUBE2 | | | | | | | | | |
| SHH)- BOC | −0.110188941 | 0.19205796 | 0.29442268 | 0.03496091 | −0.0705542 | 0.00982891 | 0.03816125 | −0.0363851 | 0.17345492 |
| SHH)- | −0.14153371 | 0.06705219 | 0.12564255 | −0.0003135 | −0.1971469 | 0.224896 | 0.11932944 | −0.0721271 | 0.326859 |
| GAS1 | | | | | | | | | |
| SHH)- | 0.138310953 | −0.0021596 | −0.0333821 | 0.16040148 | 0.02877397 | 0.07959391 | 0.00536539 | 0.15880403 | 0.10962917 |
| PTCH1 | | | | | | | | | |
| SHH)- | 0.041023589 | 0.17744843 | 0.18710679 | 0.2107022 | −0.0961977 | 0.15034303 | 0.11682455 | 0.08622944 | 0.29408594 |
| PTCH2 | | | | | | | | | |
| COL1A2)- | 0.138158916 | 0.06982916 | −0.2086319 | −0.0236807 | 0.29447082 | 0.0491718 | −0.2520433 | 0.18128744 | 0.12910105 |
| CD44 | | | | | | | | | |
| COL1A2)- | −0.40392826 | 0.42942449 | 0.45270904 | −0.2780901 | −0.1456871 | 0.69806252 | 0.42771607 | −0.3525855 | 0.43686791 |
| ITGB1 | | | | | | | | | |
| COL1A2)- | −0.368460693 | 0.37355877 | 0.43235136 | −0.4725969 | −0.3608035 | 0.6868405 | 0.38679968 | −0.3035942 | 0.42060367 |
| ITGA11 | | | | | | | | | |
| COL1A2)- | 0.194131615 | −0.3520133 | −0.2238866 | −0.0094676 | 0.13573931 | −0.1900451 | −0.1776922 | 0.17432603 | −0.2824414 |
| ITGB3 | | | | | | | | | |
| COL1A2)- | 0.143894273 | −0.1088305 | −0.0491855 | −0.073746 | −0.0445615 | −0.3417241 | −0.0974421 | 0.09067844 | −0.0436148 |
| CD36 | | | | | | | | | |
| DEFB1)- | 0.026914411 | 0.04810284 | 0.10359238 | 0.05162681 | 0.03338242 | 0.16965437 | −0.0969217 | 0.15558154 | 0.02460926 |
| CCR6 | | | | | | | | | |
| COL14A1)- | 0.086339555 | 0.11367974 | −0.1513702 | −0.3725922 | 0.2249699 | 0.03136243 | −0.26534 | 0.03098059 | 0.11658699 |
| CD44 | | | | | | | | | |
| ARF6)- | −0.116902407 | 0.07573046 | −0.04276 | −0.0113489 | 0.04036337 | 0.09701769 | 0.06021415 | 0.12495294 | −0.1355888 |
| PLD1 | | | | | | | | | |
| ARF6)- | −0.084869396 | 0.12846603 | −0.0341293 | 0.04939653 | −0.1735986 | 0.10911004 | 0.2184884 | 0.04334852 | 0.08820798 |
| SMAP1 | | | | | | | | | |
| JAM3)- | −0.104009967 | 0.07902795 | 0.00951422 | −0.1070685 | 0.00258155 | 0.22709366 | 0.1211578 | −0.0181138 | 0.04771181 |
| ITGB1 | | | | | | | | | |
| JAM3)- | −0.020525789 | −0.0182197 | −0.0166276 | −0.0020043 | 0.12056876 | −0.003046 | −0.063395 | −0.0024196 | 0.0320119 |
| ITGB2 | | | | | | | | | |
| HSP90B1)- | −0.129908275 | −0.2263722 | −0.114762 | −0.1044695 | −0.1867359 | −0.1193634 | 0.02452391 | −0.0396796 | −0.1019876 |
| TLR9 | | | | | | | | | |
| HSP90B1)- | −0.047693836 | −0.1697517 | −0.1608455 | −0.0648148 | 0.00175008 | −0.1813599 | −0.072355 | 0.03429342 | −0.0464299 |
| TLR7 | | | | | | | | | |
| HSP90B1)- | 0.02429403 | −0.162702 | −0.112969 | −0.1053177 | −0.1584466 | 0.03364776 | 0.18261455 | −0.1475103 | −0.2607488 |
| TLR4 | | | | | | | | | |
| HSP90B1)- | −0.109980722 | −0.1419739 | −0.0540329 | 0.01021758 | −0.0116205 | −0.0122631 | 0.22724179 | −0.1059026 | 0.00062395 |
| TLR1 | | | | | | | | | |
| COL6A2)- | −0.286139042 | 0.33447626 | 0.43257288 | −0.0403726 | 0.00429738 | 0.3511172 | 0.34933915 | −0.41107 | 0.36355274 |
| ITGB1 | | | | | | | | | |
| ANGPTL4)- | −0.098048956 | 0.01535009 | 0.20527597 | 0.17142535 | −0.0978611 | 0.09166093 | 0.01850118 | 0.18444157 | 0.10934635 |
| TIE1 | | | | | | | | | |
| FADD)- | 0.124129549 | 0.14475046 | 0.04871328 | 0.0896706 | −0.0328133 | 0.03468459 | 0.18889791 | −0.0775964 | −0.0609591 |
| ABCA1 | | | | | | | | | |
| FADD)- | 0.138115703 | 0.121846 | 0.04095434 | 0.14284292 | 0.08291348 | −0.0133055 | 0.10462388 | 0.01116279 | −0.0300673 |
| FAS | | | | | | | | | |
| FADD)- | −0.003188285 | 0.09777268 | 0.09643335 | 0.01181069 | 0.10529008 | −0.0237994 | −0.2290723 | −0.0136994 | 0.13673504 |
| TRADD | | | | | | | | | |
| CCL11)- | 0.147904382 | 0.17210634 | 0.21401441 | 0.21205409 | 0.25397765 | 0.08642549 | 0.29417272 | 0.24431642 | 0.4557562 |
| CCR3 | | | | | | | | | |
| CCL11)- | −0.013636104 | 0.12706406 | 0.20900062 | 0.16337504 | 0.10280043 | 0.23510656 | 0.39367838 | 0.04255625 | 0.40223801 |
| DPP4 | | | | | | | | | |
| NRTN)- | 0.00162341 | 0.22371714 | 0.21358692 | 0.12209713 | −0.006776 | −0.0369864 | 0.21253256 | −0.1766313 | 0.48827607 |
| RET | | | | | | | | | |
| NRTN)- | −0.016033406 | 0.23922037 | 0.22496007 | 0.05448946 | 0.01194337 | 0.05469118 | 0.15546287 | 0.01716452 | 0.36529144 |
| GFRA1 | | | | | | | | | |
| IGF1)- | 0.05319503 | −0.0870181 | 0.07007367 | 0.05367922 | −0.1507973 | 0.07790343 | −0.1359228 | −0.0113971 | −0.059975 |
| INSR | | | | | | | | | |
| IGF1)- | 0.098194314 | −0.1786088 | 0.02912223 | 0.02763223 | −0.1490493 | −0.1354542 | −0.1242072 | −0.1204555 | −0.1842088 |
| IGF2R | | | | | | | | | |
| HSPA4)- | −0.096270342 | −0.0549942 | 0.18367022 | −0.2121815 | 0.14671607 | −0.0658472 | 0.02897363 | −0.0588188 | −0.2019662 |
| TLR4 | | | | | | | | | |
| PCSK9)- | −0.155143103 | 0.01047077 | −0.1209914 | −0.3086327 | 0.18565933 | 0.16361219 | −0.1176717 | −0.1030103 | −0.0086137 |
| LDLR | | | | | | | | | |
| SEMA3E)- | 0.156867021 | 0.07674836 | −0.1885906 | 0.08433024 | −0.193691 | 0.11993577 | −0.1995456 | 0.30812992 | 0.39656155 |
| PLXND1 | | | | | | | | | |
| IL12A)- | −0.067228221 | 0.1046078 | 0.18936484 | 0.23008636 | −0.0798084 | 0.32841968 | 0.23301747 | 0.09654515 | 0.50744564 |
| CD28 | | | | | | | | | |
| CD14)- | −0.153057969 | −0.1394602 | 0.24483659 | 0.25897148 | −0.2086615 | 0.29258454 | −0.2437408 | 0.25373056 | 0.35877209 |
| ITGA4 | | | | | | | | | |
| CD14)- | 0.282638163 | −0.0377152 | 0.05190524 | 0.17419044 | 0.20704564 | −0.1982831 | 0.15262936 | −0.292774 | 0.1505234 |
| ITGB1 | | | | | | | | | |
| CD14)- | −0.113896004 | −0.0830837 | 0.00996342 | 0.05671501 | −0.0518687 | 0.15900494 | −0.0452687 | 0.17515464 | 0.2375771 |
| TLR6 | | | | | | | | | |
| CD14)- | −0.168255888 | −0.1034166 | 0.01892671 | 0.12484859 | −0.0198542 | 0.07247562 | 0.0151536 | 0.09204242 | 0.27973009 |
| TLR9 | | | | | | | | | |
| CD14)- | −0.379244329 | −0.3913623 | 0.57653787 | 0.47910503 | −0.3384065 | 0.53717995 | −0.4858112 | 0.57217932 | 0.57866985 |
| ITGB2 | | | | | | | | | |
| CD14)- | −0.191524901 | −0.2060936 | 0.13309929 | 0.21872263 | −0.0770833 | 0.18135932 | −0.0900034 | 0.20220996 | 0.3828517 |
| TLR4 | | | | | | | | | |
| CD14)- | −0.209417152 | −0.1934473 | 0.21539228 | 0.15220279 | −0.2316468 | 0.1623202 | −0.1803364 | 0.11994364 | 0.2634489 |
| TLR1 | | | | | | | | | |
| COL3A1)- | −0.16402703 | 0.06027957 | 0.09285976 | −0.2541834 | −0.1349222 | 0.4729262 | 0.08699817 | −0.1311319 | 0.12706224 |
| DDR2 | | | | | | | | | |
| COL3A1)- | −0.387345202 | 0.44660206 | 0.40661324 | −0.2428034 | −0.1269951 | 0.63659878 | 0.41199733 | −0.3748193 | 0.49163165 |
| ITGB1 | | | | | | | | | |
| COL3A1)- | 0.032477786 | 0.00859468 | −0.171471 | −0.2155703 | 0.14497601 | −0.0233755 | −0.0966632 | −0.0013037 | 0.03085179 |
| MAG | | | | | | | | | |
| COL3A1)- | 0.285942773 | 0.01988139 | 0.32364314 | 0.23641519 | −0.3407304 | −0.38124 | 0.37433211 | 0.37812852 | 0.00046869 |
| DDR1 | | | | | | | | | |
| IL13)- | 0.009603824 | −0.0187514 | 0.1838186 | 0.1852844 | −0.0349861 | 0.11752938 | 0.23059116 | 0.21488836 | 0.46921421 |
| IL13RA2 | | | | | | | | | |
| IL13)- | −0.066207349 | −0.0004986 | −0.0196477 | −0.1213393 | −0.034101 | 0.02030697 | 0.03739507 | 0.09028047 | −0.1590633 |
| TMEM219 | | | | | | | | | |
| IL13)- | 0.054986647 | 0.08084073 | −0.0636436 | 0.18560491 | −0.0194451 | 0.12605407 | −0.0484694 | 0.30878181 | 0.32717292 |
| IL2RG | | | | | | | | | |
| IL13)- | 0.102181142 | −0.0585696 | −0.0146069 | 0.03614726 | −0.0773617 | −0.1697254 | 0.1408628 | −0.0262915 | 0.13514464 |
| IL13RA1 | | | | | | | | | |
| IL13)- IL4R | 0.049445117 | −0.0517635 | 0.11407841 | −0.0694528 | −0.1398999 | −0.071432 | 0.00250196 | 0.01563615 | −0.1624889 |
| NLGN2)- | −0.039250962 | −0.0987644 | −0.0604797 | −0.168731 | −0.1007445 | 0.04794902 | −0.0330179 | 0.01173012 | 0.20340681 |
| NRXN1 | | | | | | | | | |
| CXCL10)- | 0.157091804 | 0.26258028 | 0.2236914 | 0.10094289 | −0.0303291 | 0.06711309 | 0.2683642 | 0.31249863 | 0.40762738 |
| DPP4 | | | | | | | | | |
| CXCL10)- | 0.098073335 | −0.0761123 | 0.15349373 | −0.1608842 | 0.04652608 | 0.07342382 | 0.06442163 | 0.02095636 | −0.4141393 |
| SDC4 | | | | | | | | | |
| CXCL10)- | 0.136195809 | 0.19118334 | 0.26527014 | 0.00485214 | 0.06490747 | 0.09940484 | 0.24682977 | 0.26016708 | 0.56333779 |
| CCR3 | | | | | | | | | |
| BMP1)- | −0.086237406 | 0.11011088 | 0.13598945 | −0.1718148 | 0.00267944 | 0.20357825 | 0.00267899 | 0.00397697 | 0.11347508 |
| BMPR2 | | | | | | | | | |
| BMP1)- | 0.246153625 | 0.1242627 | −0.1200446 | 0.00832946 | 0.21802192 | −0.2796544 | −0.1541448 | 0.1587014 | 0.10914683 |
| BMPR1A | | | | | | | | | |
| BMP1)- | 0.073479337 | 0.01580717 | −0.0742307 | −0.0602841 | 0.00265115 | −0.1118112 | −0.0135774 | 0.02663336 | −0.0199547 |
| BMPR1B | | | | | | | | | |
| FGB)- | −0.089814439 | 0.04047357 | 0.0142302 | 0.09336821 | −0.0709699 | 0.24419909 | −0.0531155 | 0.27149331 | 0.3840187 |
| ITGAM | | | | | | | | | |
| FGB)- | 0.103159549 | −0.0803342 | −0.0886205 | 0.11792466 | 0.00641128 | −0.3387611 | 0.00309337 | −0.269466 | −0.0212787 |
| ITGB1 | | | | | | | | | |
| FGB)- | 0.132530722 | −0.0063835 | −0.0520503 | 0.03053732 | −0.0334965 | 0.08051402 | 0.05499585 | −0.0084873 | 0.19078865 |
| ITGB3 | | | | | | | | | |
| FGB)- | −0.064740618 | −0.0017681 | −0.0085917 | 0.15545011 | −0.1094386 | 0.14092824 | −0.2626159 | 0.34714661 | 0.3615877 |
| ITGB2 | | | | | | | | | |
| FGB)- | −0.135665869 | −0.191111 | 0.12865593 | 0.21229077 | 0.04962748 | 0.09603368 | −0.0704263 | 0.08961894 | 0.26016334 |
| TLR4 | | | | | | | | | |
| FGA)- | −0.074851432 | 0.12933732 | −0.0660805 | 0.23227324 | −0.0861496 | 0.25901239 | −0.0442765 | 0.16541043 | 0.35321567 |
| ITGAM | | | | | | | | | |
| FGA)- | 0.003069032 | 0.08420209 | 0.0378613 | 0.00922592 | 0.06632701 | −0.0335584 | −0.2174219 | 0.30980416 | 0.42683141 |
| ITGAX | | | | | | | | | |
| FGA)- | 0.046400148 | −0.0730577 | −0.0811038 | 0.08569951 | 0.00193073 | −0.2964937 | −0.0124555 | −0.1365341 | −0.0997465 |
| ITGB1 | | | | | | | | | |
| FGA)- | 0.092092411 | 0.08438383 | −0.0747942 | −0.0533105 | 0.06937415 | −0.0515715 | 0.00886463 | 0.1886274 | 0.11184735 |
| PLAUR | | | | | | | | | |
| FGA)- | 0.035576612 | 0.02519984 | −0.0019297 | 0.19084521 | 0.02806226 | 0.24387389 | 0.04641459 | 0.05501485 | 0.26192719 |
| ITGB3 | | | | | | | | | |
| FGA)- | 0.082710699 | 0.09223082 | 0.0277814 | 0.35932797 | −0.1825721 | 0.30082315 | −0.0740828 | 0.24678955 | 0.28181284 |
| CDH5 | | | | | | | | | |
| FGA)- | 0.02634287 | 0.10366536 | −0.1666906 | −0.0214578 | 0.02949722 | −0.1300772 | −0.2326641 | 0.20903375 | 0.28618298 |
| ITGB2 | | | | | | | | | |
| FGA)- | −0.077916135 | −0.0830123 | 0.08309084 | 0.32691902 | 0.00472311 | 0.17703365 | 0.15262294 | 0.06783688 | 0.2874112 |
| TLR4 | | | | | | | | | |
| CXCL8)- | 0.16350905 | 0.12418446 | 0.10849777 | −0.0803311 | 0.07516819 | −0.0838677 | 0.11835306 | −0.036206 | −0.1678101 |
| SDC1 | | | | | | | | | |
| CXCL8)- | 0.151268153 | −0.053615 | −0.1254387 | 0.02970673 | 0.19048909 | −0.2263999 | 0.08708083 | −0.2978639 | 0.02959786 |
| SDC3 | | | | | | | | | |
| CXCL8)- | 0.271703973 | −0.1018512 | −0.073158 | 0.18419869 | 0.17874437 | −0.3353411 | 0.17324297 | −0.3050343 | 0.21206529 |
| SDC2 | | | | | | | | | |
| CXCL8)- | −0.204294586 | −0.2145892 | 0.21754801 | 0.35658962 | −0.0816933 | 0.09088366 | −0.1134752 | 0.16553171 | 0.29817606 |
| CXCR2 | | | | | | | | | |
| SEMA4C)- | 0.010034131 | −0.0769998 | 0.01564034 | 0.12031177 | −0.06269 | −0.1091518 | −0.0352884 | 0.1017036 | −0.2652688 |
| PLXNB2 | | | | | | | | | |
| CXCL11)- | −0.067433253 | 0.20076549 | 0.23687224 | 0.11221327 | −0.0992402 | 0.21095087 | 0.19451684 | 0.4320337 | 0.38249196 |
| DPP4 | | | | | | | | | |
| CXCL11)- | −0.001012638 | 0.18725522 | 0.20774227 | 0.1720724 | −0.0059996 | 0.37264912 | 0.34535701 | 0.30846225 | 0.53121055 |
| CCR3 | | | | | | | | | |
| PRND)- | 0.001477584 | −0.1262515 | 0.02172266 | −0.0502522 | −0.1545581 | −0.2164466 | −0.1578425 | −0.1339616 | −0.226258 |
| RPSA | | | | | | | | | |
| HAS2)- | −0.017266167 | −0.0135068 | 0.09596854 | −0.0273524 | 0.0322507 | 0.23295436 | 0.09026243 | 0.08605945 | 0.29325772 |
| HMMR | | | | | | | | | |
| HAS2)- | 0.010700869 | 0.01452372 | 0.07606654 | 0.00490738 | −0.1072373 | −0.0258188 | −0.2480537 | −0.1692247 | 0.08185415 |
| CD44 | | | | | | | | | |
| NPTX1)- | −0.125466933 | 0.26112069 | 0.26974256 | −0.2021331 | −0.1807825 | 0.18123492 | 0.14278745 | 0.07082091 | 0.33027561 |
| NPTXR | | | | | | | | | |
| RGMB)- | −0.069338976 | −0.0791226 | −0.0404692 | −0.0326627 | 0.06605953 | 0.0795364 | −0.0378225 | −0.2983695 | 0.02127002 |
| BMPR2 | | | | | | | | | |
| RGMB)- | −0.04788543 | 0.13214367 | 0.02174802 | −0.1022274 | −0.1280382 | 0.11567023 | −0.0284843 | −0.0482957 | −0.0287765 |
| NEO1 | | | | | | | | | |
| RGMB)- | 0.013383363 | 0.2978683 | 0.2725173 | 0.05388464 | −0.126253 | 0.32209738 | 0.15492598 | 0.00593631 | 0.34334735 |
| BMPR1B | | | | | | | | | |
| F2)- GP1BB | 0.093208999 | 0.34339029 | 0.29255615 | 0.17390438 | 0.07113029 | 0.04502566 | 0.09808165 | 0.05986417 | 0.56251547 |
| F2)- F2R | 0.11285561 | 0.15489337 | −0.0157272 | 0.07806888 | 0.05038313 | 0.08105514 | −0.2157436 | 0.14341909 | 0.28145546 |
| F2)- THBD | −0.022069382 | 0.03387914 | 0.13632793 | −0.0077847 | −0.0395437 | 0.10756045 | 0.02714314 | 0.11110346 | 0.09985236 |
| F2)- GP1BA | −0.003223925 | 0.26161793 | 0.28756343 | 0.19565033 | 0.10564646 | 0.20617631 | 0.11371473 | 0.26220111 | 0.48213326 |
| CCL19)- | 0.003879979 | −0.0197598 | 0.1251303 | 0.07204494 | 0.06150051 | −0.0350034 | 0.12255198 | 0.17708297 | 0.30453403 |
| CCRL2 | | | | | | | | | |
| CLCF1)- | −0.077742533 | 0.16247506 | 0.13616054 | 0.10204922 | −0.0961095 | 0.11526155 | 0.11423971 | 0.0953578 | 0.09269292 |
| CRLF1 | | | | | | | | | |
| CLCF1)- | −0.107957967 | 0.14573235 | 0.09407368 | 0.11565483 | 0.0202564 | −0.0398467 | −0.0481429 | 0.02652648 | 0.22950421 |
| IL6ST | | | | | | | | | |
| CSHL1)- | −0.140429936 | 0.05400562 | 0.12236963 | 0.18592243 | −0.1306492 | 0.14619504 | 0.01796735 | 0.13459006 | 0.41155862 |
| GHR | | | | | | | | | |
| LPL)- CD44 | −0.060867815 | −0.1319761 | 0.03749373 | −0.0084505 | −0.216266 | −0.1598119 | −0.1893582 | −0.0894356 | −0.0519517 |
| LPL)- SDC1 | 0.050803244 | −0.1787955 | 0.00209929 | 0.16399952 | −0.091084 | −0.0287493 | 0.02761902 | −0.048122 | −0.2034241 |
| EFEMP2)- | 0.023149905 | 0.04707209 | 0.20254553 | −0.1169779 | −0.0818469 | −0.042655 | 0.1048554 | −0.0358026 | 0.1847683 |
| AQP1 | | | | | | | | | |
| EFEMP2)- | −0.078567288 | 0.01794976 | 0.25373351 | −0.0503153 | −0.1761162 | 0.14670639 | 0.23910907 | −0.1354083 | 0.24441886 |
| PLSCR4 | | | | | | | | | |
| ADAM17)- | 0.052816883 | 0.03231216 | 0.12040772 | −0.0587576 | 0.02868147 | 0.04508051 | 0.07250438 | 0.07292913 | −0.0568763 |
| MUC1 | | | | | | | | | |
| ADAM17)- | −0.037716472 | 0.07710901 | 0.1392064 | −0.2309991 | −0.0667594 | 0.17980674 | −0.0164835 | −0.0604126 | 0.12831457 |
| ITGB1 | | | | | | | | | |
| ADAM17)- | −0.073825966 | 0.13298483 | 0.02814775 | 0.12788163 | −0.1191127 | 0.00769534 | 0.08698174 | 0.0634966 | 0.35202946 |
| RHBDF2 | | | | | | | | | |
| ADAM17)- | 0.022544295 | 0.02208042 | 0.13146989 | 0.00260915 | 0.15845698 | −0.0272698 | −0.0695913 | −0.0206458 | 0.06887814 |
| IL6R | | | | | | | | | |
| ADAM17)- | −0.012077209 | 0.10426251 | 0.15622283 | 0.00493703 | −0.046191 | −0.0198111 | −0.0253565 | −0.1396813 | 0.39690014 |
| MET | | | | | | | | | |
| VEGFB)- | −0.040844943 | −0.1953574 | −0.0811124 | 0.09093679 | 0.11041792 | −0.2031163 | 0.11094866 | −0.2313611 | 0.11069108 |
| RET | | | | | | | | | |
| LEP)- LEPR | −0.065481592 | 0.01985586 | 0.19889696 | 0.23748635 | 0.12388749 | 0.34786784 | 0.28118743 | 0.16955731 | 0.44066039 |
| GH1)- GHR | −0.014725995 | −0.0398549 | 0.09019776 | 0.21895929 | −0.0279325 | 0.18179435 | 0.17609613 | 0.19813765 | 0.39779508 |
| GNAI2)- | −0.038070258 | −0.2416393 | 0.00697834 | −0.1325688 | −0.0553667 | 0.01960368 | −0.0693962 | 0.16664837 | 0.06207268 |
| S1PR5 | | | | | | | | | |
| GNAI2)- | 0.263371236 | −0.2233895 | −0.0862665 | 0.03642361 | −0.0173448 | 0.3575249 | −0.0207034 | 0.18152968 | 0.12911277 |
| EDNRA | | | | | | | | | |
| GNAI2)- | −0.343812302 | −0.392318 | 0.38352322 | −0.0518672 | 0.05687386 | 0.01570185 | 0.11823553 | −0.0102442 | 0.06726894 |
| C5AR1 | | | | | | | | | |
| GNAI2)- | −0.287926041 | −0.2602572 | −0.0274536 | −0.1582352 | −0.0384132 | −0.1798852 | 0.01706519 | −0.1479398 | −0.1046004 |
| ADRA2B | | | | | | | | | |
| GNAI2)- | 0.134857629 | −0.2712296 | 0.08870586 | −0.1048182 | −0.1301568 | 0.20265911 | −0.0388484 | 0.09457598 | 0.03007241 |
| F2R | | | | | | | | | |
| GNAI2)- | −0.254798791 | −0.3263145 | 0.27253239 | −0.0849337 | −0.0365621 | 0.09505438 | −0.0520682 | 0.16195497 | 0.03869633 |
| FPR1 | | | | | | | | | |
| GNAI2)- | −0.136650162 | −0.3195814 | −0.041851 | −0.1934286 | 0.12641043 | −0.0441283 | 0.15924653 | −0.0415946 | −0.1115106 |
| S1PR3 | | | | | | | | | |
| GNAI2)- | 0.067720983 | −0.2720503 | 0.02700691 | −0.2227866 | −0.0532859 | 0.0944329 | 0.16536117 | −0.0487671 | 0.15010157 |
| UNC5B | | | | | | | | | |
| GNAI2)- | 0.195844076 | −0.2423087 | 0.0529491 | −0.0295718 | 0.08616543 | 0.17956973 | 0.23246126 | −0.0921897 | 0.39855174 |
| CAV1 | | | | | | | | | |
| GNAI2)- | 0.215468123 | −0.036469 | −0.0632615 | 0.11495411 | −0.011001 | 0.1454809 | 0.10035859 | 0.03586761 | 0.29450018 |
| PTPRU | | | | | | | | | |
| GNAI2)- | −0.051329358 | −0.290275 | −0.1413625 | −0.106594 | −0.0376693 | −0.0280708 | −0.0241422 | 0.08740845 | −0.0206899 |
| TBXA2R | | | | | | | | | |
| GNAI2)- | −0.198975391 | −0.2864019 | 0.10076866 | −0.1200069 | −0.1954616 | 0.0176502 | 0.02093003 | 0.07347989 | 0.03869633 |
| EDNRB | | | | | | | | | |
| GNAI2)- | −0.122026966 | −0.3105553 | −0.0934527 | −0.1489257 | 0.08629102 | −0.1022823 | −0.0238858 | −0.0446025 | −0.0164364 |
| CXCR2 | | | | | | | | | |
| GNAI2)- | −0.098819655 | −0.3144257 | −0.0249348 | −0.1718599 | −0.0622098 | −0.2596001 | −0.0383751 | −0.0805323 | −0.0220754 |
| DRD2 | | | | | | | | | |
| GNAI2)- | −0.088417983 | −0.1715457 | 0.10972754 | −0.2100975 | −0.1640098 | 0.08904261 | −0.1364759 | 0.18458839 | 0.12797108 |
| ADCY7 | | | | | | | | | |
| GNAI2)- | −0.067933355 | −0.3686265 | −0.1119555 | −0.2801665 | −0.1692769 | −0.1770508 | −0.1038469 | −0.0385715 | −0.1093867 |
| AGTR2 | | | | | | | | | |
| ANGPT2)- | −0.160525943 | 0.13152891 | 0.1481342 | 0.27224982 | −0.0051819 | 0.27342064 | 0.12990453 | 0.13792892 | 0.46116632 |
| TIE1 | | | | | | | | | |
| TLN1)- | −0.044512508 | 0.07791536 | 0.02128238 | −0.031994 | −0.0064107 | 0.06468262 | −0.0271582 | −0.0201515 | −0.1079085 |
| ITGB3 | | | | | | | | | |
| AGTRAP)- | −0.078851168 | 0.07271886 | −0.0350145 | 0.19501584 | −0.0243613 | −0.1086677 | −0.134118 | −0.0981276 | 0.19708676 |
| RACK1 | | | | | | | | | |
| PKM)- | −0.026229297 | −0.141513 | 0.25090593 | −0.1526706 | 0.20246425 | 0.00715636 | 0.00873854 | 0.18000319 | 0.2651772 |
| CD44 | | | | | | | | | |
| MMP1)- | 0.160091581 | 0.19071014 | −0.2136125 | 0.15915352 | −0.3435871 | −0.1314478 | 0.42837871 | −0.0117144 | −0.309785 |
| CD44 | | | | | | | | | |
| LAMA3)- | −0.356291148 | 0.26643095 | 0.12869094 | 0.23567889 | 0.20827475 | −0.3880307 | −0.0677293 | 0.12660163 | −0.090549 |
| ITGB1 | | | | | | | | | |
| MTMR4)- | −0.045309404 | 0.29266013 | −0.1039054 | 0.11515411 | −0.0958214 | −0.0608775 | 0.03415391 | 0.078398 | 0.11590114 |
| SMAD3 | | | | | | | | | |
| WNT11)- | 0.1055012 | 0.24766735 | 0.11658116 | 0.05877191 | 0.0304177 | 0.13964157 | 0.03956952 | 0.07352237 | 0.14066915 |
| KLRG2 | | | | | | | | | |
| PSEN1)- | −0.055973243 | 0.02667073 | 0.05374599 | 0.01097652 | −0.075962 | 0.09178791 | 0.05059677 | −0.0510735 | −0.1485415 |
| NOTCH4 | | | | | | | | | |
| PSEN1)- | −0.277894229 | 0.02540805 | −0.0397748 | 0.0895111 | −0.1036366 | 0.08814332 | 0.20851069 | 0.07105152 | −0.0097459 |
| CD44 | | | | | | | | | |
| BGN)- | −0.053516423 | −0.0327236 | −0.1002402 | −0.3311075 | 0.17079867 | −0.0963048 | −0.2338533 | −0.0494459 | 0.05705163 |
| TLR4 | | | | | | | | | |
| BGN)- | 0.098748651 | −0.023297 | −0.1784725 | −0.1316542 | 0.24129162 | −0.1166068 | −0.1603638 | 0.04985177 | 0.01774878 |
| TLR1 | | | | | | | | | |
| CSF1)- | −0.063939851 | 0.01573935 | 0.26447298 | −0.0098231 | 0.02832782 | 0.01699611 | 0.1571882 | 0.15507546 | 0.12775375 |
| SIRPA | | | | | | | | | |
| P4HB)- | −0.048149424 | −0.143773 | −0.1464312 | −0.0997415 | −0.0821985 | 0.0143021 | −0.0300606 | −0.050071 | −0.2533728 |
| MTTP | | | | | | | | | |
| JAG2)- | 0.098027264 | 0.16136249 | 0.02308874 | −0.0781262 | −0.0774228 | 0.30795882 | −0.0282875 | −0.0282972 | 0.10877627 |
| NOTCH4 | | | | | | | | | |
| CTHRC1)- | 0.2389583 | −0.1405369 | 0.09941837 | 0.1426073 | −0.1665353 | −0.2537105 | 0.22034447 | 0.36493489 | −0.3268658 |
| FZD5 | | | | | | | | | |
| CTHRC1)- | −0.02569883 | −0.0456536 | 0.05830798 | 0.05170058 | 0.03336751 | 0.10212461 | 0.09669363 | 0.04180894 | 0.060145 |
| FZD6 | | | | | | | | | |
| CTHRC1)- | 0.004588688 | 0.05499263 | −0.0904247 | −0.0958234 | 0.02735412 | −0.0950234 | −0.0426521 | 0.09239204 | 0.11166964 |
| FZD3 | | | | | | | | | |
| FBLN1)- | −0.146250796 | 0.0551094 | 0.08298181 | −0.1364849 | −0.0644509 | 0.17761024 | −0.0454924 | −0.2784524 | −0.0066642 |
| ITGB1 | | | | | | | | | |
| CALCA)- | −0.038596009 | 0.13834544 | 0.14485258 | 0.1312197 | −0.0450861 | 0.18991495 | 0.09193901 | 0.11734669 | 0.49513292 |
| CALCRL | | | | | | | | | |
| CALCA)- | −0.104243967 | 0.07011337 | 0.1402484 | 0.19707568 | 0.13586581 | 0.21319357 | 0.19489728 | 0.16623869 | 0.45184035 |
| GPR84 | | | | | | | | | |
| CALCA)- | −0.021660523 | 0.07444591 | 0.10257756 | 0.36234124 | 0.15390105 | 0.23148018 | 0.09693739 | 0.20187666 | 0.4380022 |
| CALCR | | | | | | | | | |
| CALCA)- | −0.059950256 | 0.08171895 | 0.20518209 | 0.16704394 | 0.15413745 | 0.21161884 | 0.17730518 | 0.056363 | 0.44936148 |
| ADRB3 | | | | | | | | | |
| COL4A5)- | 0.051607214 | −0.1625404 | −0.0005567 | 0.02633279 | −0.0760893 | −0.0617953 | 0.14171568 | 0.16633442 | −0.0287134 |
| CD47 | | | | | | | | | |
| COL4A5)- | −0.250625547 | 0.05614593 | −0.112362 | −0.2866699 | −0.1222598 | 0.23933754 | 0.1345908 | −0.2711001 | 0.25053387 |
| ITGB1 | | | | | | | | | |
| SLIT3)- | 0.098079165 | −0.1336056 | −0.0164623 | 0.19667676 | 0.08278563 | 0.16227151 | −0.0673547 | 0.10877489 | 0.37326584 |
| ROBO1 | | | | | | | | | |
| COL7A1)- | −0.093654139 | 0.00121004 | 0.04635342 | −0.2971337 | −0.1093736 | 0.39262535 | 0.22073054 | −0.2823957 | 0.31711168 |
| ITGB1 | | | | | | | | | |
| GHRL)- | −0.07912255 | 0.14580824 | 0.16291557 | 0.17515316 | −0.0119627 | 0.05669941 | 0.08523152 | −0.0344766 | 0.44623265 |
| PTGER3 | | | | | | | | | |
| GHRL)- | −0.079153671 | 0.05207806 | 0.25816898 | 0.19833258 | −0.1307064 | 0.28715928 | 0.08886787 | 0.03803116 | 0.35613348 |
| TBXA2R | | | | | | | | | |
| HSP90AA1)- | −0.048883853 | 0.00211499 | 0.02536615 | 0.00132699 | 0.0442073 | −0.056615 | −0.1050472 | −0.0906111 | −0.3290653 |
| ITGB3 | | | | | | | | | |
| HSP90AA1)- | 0.106290073 | −0.0061282 | 0.05979277 | 0.0121948 | 0.06308936 | 0.14659891 | 0.0785893 | −0.0654877 | −0.2012101 |
| FGFR3 | | | | | | | | | |
| FGG)- | −0.078645203 | −0.063073 | −0.1344853 | −0.0104994 | 0.05081349 | −0.168296 | −0.0783858 | −0.2332704 | −0.146777 |
| ITGB1 | | | | | | | | | |
| FGG)- | −0.093845005 | 0.13563646 | 0.30087776 | 0.15336013 | −0.0625298 | 0.17400953 | 0.10563715 | 0.01694526 | 0.49258143 |
| ITGB3 | | | | | | | | | |
| FGG)- | −0.007382859 | 0.12075989 | −0.1391812 | 0.03379655 | 0.01784676 | 0.08932734 | −0.1115799 | 0.18822116 | 0.25067997 |
| ITGB2 | | | | | | | | | |
| FGG)- | −0.162302828 | 0.06849849 | 0.18950775 | 0.16409322 | −0.0563276 | 0.14896981 | 0.11545877 | 0.1297477 | 0.43366157 |
| TLR4 | | | | | | | | | |
| CXCL14)- | 0.086685239 | 0.11946756 | −0.0997781 | −0.1300228 | 0.17287483 | −0.013921 | −0.2460117 | 0.12893701 | 0.23752109 |
| CXCR4 | | | | | | | | | |
| EDN3)- | 0.100031814 | 0.17475222 | 0.02500301 | 0.10984317 | 0.05890488 | 0.16854173 | 0.12812353 | 0.04409841 | 0.28823688 |
| EDNRB | | | | | | | | | |
| EDN3)- | −0.001483256 | 0.1754939 | 0.24696421 | 0.21118963 | 0.11351969 | 0.11529512 | 0.33343394 | −0.0107844 | 0.4069093 |
| KEL | | | | | | | | | |
| RTN4)- | −0.09861229 | −0.1860788 | 0.04780823 | −0.2349392 | −0.0285559 | 0.13386545 | 0.11389153 | −0.0373207 | −0.1183797 |
| TNFRSF19 | | | | | | | | | |
| RTN4)- | 0.041533186 | −0.2039072 | 0.07229862 | −0.165514 | −0.0340809 | 0.12206912 | 0.14611276 | 0.00511211 | 0.01377955 |
| GJB2 | | | | | | | | | |
| WNT7B)- | 0.079252215 | 0.20739699 | 0.18675547 | 0.12670472 | 0.03233421 | 0.03572375 | 0.06260614 | −0.0035448 | 0.18828992 |
| FZD4 | | | | | | | | | |
| WNT7B)- | 0.136711907 | 0.06634725 | 0.0502798 | 0.21122329 | −0.0023875 | 0.0265591 | −0.0584479 | 0.09141218 | 0.17648825 |
| TMED5 | | | | | | | | | |
| PTN)- | 0.088649556 | −0.166832 | 0.06891883 | −0.0881242 | −0.0920475 | −0.1194318 | 0.01988224 | −0.0506017 | −0.247576 |
| SDC1 | | | | | | | | | |
| PTN)- | −0.119235815 | 0.09268749 | −0.0893741 | 0.17673977 | 0.26006463 | −0.0123112 | 0.02030695 | −0.0151401 | 0.06675923 |
| SDC3 | | | | | | | | | |
| PTN)- | 0.175564892 | 0.32071283 | 0.25318592 | 0.10312765 | 0.077722 | 0.24906867 | 0.38131466 | 0.07700433 | 0.46669723 |
| PTPRZ1 | | | | | | | | | |
| PTN)- | 0.090871971 | 0.16124435 | 0.18277588 | 0.0699552 | −0.0177216 | 0.06785478 | 0.03036683 | 0.07318301 | 0.09249053 |
| PTPRS | | | | | | | | | |
| COL9A3)- | 0.132414839 | −0.2175199 | −0.2429416 | 0.12563009 | 0.13319896 | −0.1146239 | 0.06622922 | −0.3434499 | 0.10413783 |
| ITGB1 | | | | | | | | | |
| COL9A3)- | −0.052393352 | 0.0537006 | 0.30886211 | −0.0401879 | 0.04112257 | 0.18506749 | 0.02032161 | 0.00830111 | 0.2339523 |
| MAG | | | | | | | | | |
| SLPI)- | −0.118892917 | 0.26969169 | 0.0882755 | 0.2329845 | 0.08078136 | −0.0913907 | −0.0743556 | −0.1887638 | −0.1783877 |
| PLSCR4 | | | | | | | | | |
| HMGB1)- | 0.05066801 | −0.0538183 | 0.10227416 | −0.0489569 | 0.11433175 | 0.13884137 | 0.07114201 | 0.07582203 | 0.3490019 |
| SDC1 | | | | | | | | | |
| HMGB1)- | −0.083830315 | −0.0296392 | −0.0206499 | −0.0231301 | −0.0831212 | −0.2171781 | −0.0801873 | −0.1975202 | −0.4079209 |
| TLR9 | | | | | | | | | |
| HMGB1)- | 0.142021993 | −0.1289328 | 0.02833024 | −0.1795549 | 0.03695498 | 0.13388918 | 0.01009736 | −0.0448873 | 0.0995565 |
| THBD | | | | | | | | | |
| HMGB1)- | 0.028556363 | 0.01232526 | −0.1816421 | −0.0711539 | −0.2227383 | −0.0180454 | −0.0026926 | 0.00437658 | −0.4459472 |
| TLR4 | | | | | | | | | |
| HMGB1)- | 0.080428153 | 0.09333779 | −0.1073248 | 0.10400339 | −0.1749443 | 0.0831494 | 0.07497546 | −0.0305294 | −0.1034814 |
| CXCR4 | | | | | | | | | |
| HMGB1)- | 0.052803986 | 0.0817411 | 0.01776565 | 0.24461656 | −0.0668446 | 0.08193806 | −0.1479688 | −0.0225548 | −0.2508195 |
| AGER | | | | | | | | | |
| HMGB1)- | 0.153076738 | 0.13202567 | −0.0926916 | 0.12275997 | −0.0999889 | 0.16598192 | 0.17757333 | −0.2589767 | −0.449962 |
| CD163 | | | | | | | | | |
| FYN)- SPN | −0.014323587 | 0.11102548 | 0.11681113 | −0.0225404 | −0.0862257 | 0.06547929 | 0.12234938 | −0.105597 | 0.02349915 |
| FN1)- | 0.251583326 | 0.26403477 | −0.2012854 | −0.1336253 | −0.0529848 | −0.0011044 | −0.2612254 | 0.22754196 | 0.33612547 |
| ITGA4 | | | | | | | | | |
| FN1)- | 0.125166696 | −0.2609517 | −0.0408729 | 0.02020385 | 0.18631454 | −0.1638576 | 0.03348202 | 0.06354901 | −0.1609645 |
| ITGB3 | | | | | | | | | |
| FN1)- | −0.035601189 | −0.0389658 | −0.028397 | −0.2564819 | 0.34329335 | −0.1768445 | −0.1091195 | 0.03257217 | 0.0675853 |
| C5AR1 | | | | | | | | | |
| FN1)- | 0.111443101 | 0.03540969 | 0.06624953 | 0.12315872 | −0.0749532 | −0.1978335 | 0.13200108 | 0.24458661 | −0.0508912 |
| ITGA6 | | | | | | | | | |
| FN1)- | 0.020058335 | −0.1953163 | −0.1978913 | −0.0027682 | −0.0043936 | −0.1634991 | −0.1256202 | −0.0229502 | −0.0553586 |
| ITGA8 | | | | | | | | | |
| FN1)- DPP4 | −0.085610445 | −0.1877434 | −0.127881 | 0.00785652 | 0.13689572 | 0.05426437 | −0.031154 | −0.1056507 | −0.0787022 |
| FN1)- CD44 | 0.039525449 | −0.1709669 | 0.05982798 | −0.0208364 | 0.23787274 | −0.0488734 | −0.1287138 | 0.07598682 | −0.0153614 |
| FN1)- | −0.238797794 | 0.29805009 | 0.36007218 | −0.165523 | −0.0018865 | 0.65120053 | 0.34416596 | −0.2664567 | 0.36006099 |
| ITGB1 | | | | | | | | | |
| FN1)- | −0.225372292 | −0.1451305 | −0.0520732 | 0.03220529 | −0.0560414 | −0.0618448 | −0.0397567 | −0.2469143 | −0.0421471 |
| COL13A1 | | | | | | | | | |
| FN1)- MAG | 0.006336772 | 0.06867541 | −0.0836127 | −0.1461958 | 0.10887675 | 0.0307569 | −0.0065077 | −0.0332805 | 0.13154487 |
| COL18A1)- | −0.113489524 | 0.17125444 | 0.15492104 | −0.2093783 | −0.1721606 | 0.12788541 | 0.04982776 | −0.4395931 | 0.13332533 |
| ITGB1 | | | | | | | | | |
| COL18A1)- | −0.088095916 | −0.0947801 | 0.09893143 | 0.10027686 | −0.1420562 | 0.12566041 | 0.03060844 | −0.1076705 | 0.15741409 |
| ITGB3 | | | | | | | | | |
| RSPO3)- | −0.05674712 | 0.16747351 | 0.21876549 | 0.19894132 | −0.0111857 | 0.21368802 | 0.03511202 | 0.10273506 | 0.13133435 |
| LGR6 | | | | | | | | | |
| RSPO3)- | 0.028007134 | −0.0406298 | 0.25801424 | 0.07693041 | −0.2170448 | −0.1479215 | 0.09641987 | 0.0899566 | −0.1382777 |
| LGR4 | | | | | | | | | |
| RSPO3)- | 0.174324708 | −0.2003906 | 0.13712954 | −0.1064316 | −0.1964946 | −0.1957152 | 0.12486635 | 0.11022254 | −0.4024314 |
| SDC4 | | | | | | | | | |
| RSPO3)- | 0.003250198 | 0.12836499 | 0.20140891 | 0.00773579 | −0.0909969 | 0.16344228 | 0.10188233 | 0.15877057 | 0.20267889 |
| FZD8 | | | | | | | | | |
| ADAM15)- | −0.072926736 | 0.03978932 | 0.07950236 | −0.3150172 | −0.113608 | 0.16976788 | 0.10206341 | −0.1068305 | 0.01567189 |
| ITGB1 | | | | | | | | | |
| ADAM15)- | −0.077388279 | −0.0026101 | −0.0007734 | 0.1371491 | 0.13643789 | 0.05408004 | 0.24738596 | 0.05110824 | 0.01291254 |
| ITGB3 | | | | | | | | | |
| CALM1)- | −0.033817805 | −0.1861227 | −0.1396255 | 0.11261868 | −0.031667 | −0.106978 | −0.2574005 | 0.02429013 | −0.0001245 |
| KCNQ3 | | | | | | | | | |
| CALM1)- | −0.050349415 | −0.0444041 | −0.0336255 | 0.00063717 | 0.02575651 | −0.1699116 | −0.4136464 | 0.11043875 | −0.1347023 |
| CALCR | | | | | | | | | |
| CALM1)- | −0.043991978 | −0.1084688 | 0.01409213 | 0.08680788 | −0.0819412 | −0.1842786 | −0.2372347 | 0.06529025 | 0.00275357 |
| GRM4 | | | | | | | | | |
| CALM1)- | −0.08380615 | −0.1588495 | 0.05590746 | 0.00360186 | −0.04536 | 0.14988962 | −0.1738027 | −0.0782011 | 0.12209465 |
| CRHR1 | | | | | | | | | |
| CALM1)- | 0.160491549 | −0.0042421 | 0.02609985 | −0.2932428 | −0.2945142 | 0.39901201 | −0.0246093 | −0.0800814 | 0.00305529 |
| MYLK | | | | | | | | | |
| CALM1)- | −0.000487847 | 0.04784044 | −0.1180113 | 0.00168741 | 0.07167784 | −0.1032055 | −0.1075306 | 0.0070299 | 0.0383273 |
| HMMR | | | | | | | | | |
| CALM1)- | −0.108900012 | −0.0266653 | −0.0047606 | 0.06568889 | −0.0662842 | −0.0876337 | −0.1569799 | 0.09750931 | 0.06580593 |
| SCN10A | | | | | | | | | |
| CALM1)- | −0.037844149 | −0.1366106 | −0.0569062 | −0.1218509 | 0.11793455 | −0.0407306 | −0.2280191 | 0.02908648 | 0.007171 |
| AQP6 | | | | | | | | | |
| CALM1)- | 0.100142986 | −0.0500393 | 0.11094977 | 0.02368094 | 0.07618438 | 0.10461001 | −0.0722453 | 0.01301547 | −0.090663 |
| PTPRA | | | | | | | | | |
| CALM1)- | 0.057146666 | −0.106115 | −0.089838 | −0.047186 | 0.07271477 | −0.1523163 | −0.1499234 | −0.1541807 | 0.0292992 |
| SCN4A | | | | | | | | | |
| COL9A1)- | −0.010727325 | −0.2413686 | −0.20852 | −0.1533126 | −0.0717601 | −0.0235101 | −0.110134 | −0.2804364 | −0.0182732 |
| ITGB1 | | | | | | | | | |
| COL9A1)- | −0.072298179 | 0.25530688 | 0.04820578 | 0.10770089 | 0.02103837 | 0.34963325 | 0.00882542 | 0.12823431 | 0.41755206 |
| MAG | | | | | | | | | |
| GIP)- GIPR | 0.126466762 | 0.07269092 | 0.21447045 | −0.0833092 | 0.02675508 | 0.07598419 | 0.05663247 | 0.1198753 | 0.04212376 |
| GIP)- DPP4 | 0.109645923 | 0.3124328 | 0.12496062 | 0.08892474 | 0.0231725 | 0.15129344 | 0.20964079 | 0.19678126 | 0.45801538 |
| GIP)- | 0.06132893 | 0.30964066 | 0.18654908 | 0.09687269 | −0.1347929 | 0.2418588 | 0.04593798 | 0.14154444 | 0.32731161 |
| GPR84 | | | | | | | | | |
| APOA4)- | 0.034884337 | −0.0914484 | 0.16521121 | −0.0375363 | −0.1504323 | −0.1687405 | 0.09420794 | 0.04848077 | −0.1280685 |
| LDLR | | | | | | | | | |
| SEMA4F)- | 0.016827864 | 0.15079076 | 0.07072084 | 0.2596376 | −0.0340764 | 0.0079701 | −0.0059574 | −0.2650777 | 0.21396213 |
| NRP2 | | | | | | | | | |
| GAD1)- | 0.087231506 | 0.26375346 | 0.01040486 | 0.06200986 | 0.13519166 | 0.16662344 | 0.24905646 | 0.09747394 | 0.4495833 |
| GRM4 | | | | | | | | | |
| ITIH2)- | −0.076311231 | 0.12465367 | 0.16833303 | 0.10834986 | 0.03358489 | 0.06256988 | −0.0808967 | 0.07786131 | 0.26475361 |
| FCER1A | | | | | | | | | |
| CCL20)- | −0.001092999 | 0.01511654 | 0.13815361 | 0.03729417 | −0.1247174 | 0.2063367 | 0.10280376 | 0.06307878 | 0.29160814 |
| CCR6 | | | | | | | | | |
| FGF17)- | −0.063936108 | 0.12742958 | 0.10372265 | 0.21706763 | −0.1025156 | 0.23720506 | 0.21292541 | 0.03151146 | 0.26956319 |
| FGFR2 | | | | | | | | | |
| FGF17)- | 0.012646948 | 0.20990319 | −0.0826983 | 0.09335091 | 0.05441993 | −0.0051951 | −0.0656183 | −0.122913 | 0.31555621 |
| FGFR1 | | | | | | | | | |
| COL4A2)- | 0.082337762 | −0.1880406 | −0.2303782 | −0.0624542 | −0.0404785 | −0.1986293 | −0.1604918 | 0.08627062 | −0.1426707 |
| ITGB3 | | | | | | | | | |
| LAMC3)- | −0.088902914 | −0.0717726 | −0.0118629 | −0.1132068 | −0.019446 | 0.00891871 | 0.08023411 | 0.03607752 | −0.1330793 |
| ITGA6 | | | | | | | | | |
| LAMC3)- | −0.038844309 | −0.1720476 | −0.2418221 | −0.0481227 | −0.0092058 | −0.2101031 | −0.1349538 | −0.1336634 | −0.171055 |
| ITGB1 | | | | | | | | | |
| CCL28)- | 0.022865809 | 0.11764827 | 0.11301146 | 0.16656559 | 0.03459229 | 0.02433375 | −0.1135046 | −0.1708476 | −0.2334178 |
| CCR3 | | | | | | | | | |
| CXCL9)- | 0.132800719 | 0.22679788 | 0.06354944 | 0.18845298 | 0.12415451 | 0.15528551 | 0.23418987 | 0.19414094 | 0.39898528 |
| CCR3 | | | | | | | | | |
| TGM2)- | −0.066444521 | 0.04944152 | −0.0478038 | −0.1063011 | 0.0623344 | −0.0512824 | −0.0815958 | 0.01793655 | 0.13625758 |
| ITGA4 | | | | | | | | | |
| TGM2)- | 0.055039986 | −0.0133256 | 0.13034111 | −0.3256379 | −0.1779006 | 0.09600225 | 0.13452615 | −0.2359944 | 0.41977822 |
| ITGB1 | | | | | | | | | |
| TGM2)- | 0.119251979 | 0.05769969 | −0.0590599 | 0.17864592 | 0.04746909 | −0.1080063 | 0.01562122 | 0.10018259 | 0.04734697 |
| ITGB3 | | | | | | | | | |
| TGM2)- | 0.160510409 | 0.06208898 | 0.20214223 | −0.0503823 | −0.041433 | 0.05261146 | 0.36277301 | 0.32198514 | −0.02371 |
| SDC4 | | | | | | | | | |
| TGM2)- | −0.045310424 | 0.08969114 | −0.0168088 | 0.11828566 | 0.05575517 | −0.0904238 | −0.084379 | 0.18114953 | −0.1022808 |
| TBXA2R | | | | | | | | | |
| TGM2)- | 0.307572205 | −0.1529263 | 0.18283693 | −0.0123824 | −0.1254652 | 0.00393707 | 0.27453387 | 0.20443148 | −0.031291 |
| ADGRG1 | | | | | | | | | |
| CNTF)- | 0.022673331 | 0.10628865 | 0.07475019 | 0.0095621 | 0.13929846 | 0.14133836 | 0.12088637 | −0.0235298 | 0.32781396 |
| IL6R | | | | | | | | | |
| CNTF)- | −0.035024406 | 0.08610952 | −0.0147014 | 0.02403772 | −0.1332653 | 0.12346643 | −0.0683583 | 0.00138711 | 0.07918565 |
| IL6ST | | | | | | | | | |
| AGT)- | 0.114316109 | 0.086053 | −0.0078469 | 0.24191347 | 0.02692707 | 0.00727133 | 0.1716617 | 0.10551238 | 0.3537688 |
| AGTR1 | | | | | | | | | |
| AGT)- | 0.009626177 | 0.06976574 | −0.0555868 | 0.18564314 | −0.0455367 | 0.0280039 | 0.22305778 | 0.11251636 | 0.3156114 |
| AGTR2 | | | | | | | | | |
| DLL1)- | 0.014903195 | 0.06863237 | 0.19717478 | −0.0711165 | −0.1327188 | 0.1546141 | −0.0855571 | 0.11994047 | 0.17759382 |
| NOTCH4 | | | | | | | | | |
| THBS2)- | 0.195934031 | 0.10309176 | −0.1621949 | −0.0761741 | −0.0018613 | 0.01995084 | −0.21271 | 0.20664705 | 0.25940974 |
| ITGA4 | | | | | | | | | |
| THBS2)- | −0.289282236 | 0.34163846 | 0.39430423 | −0.4027879 | −0.1758678 | 0.64902239 | 0.39622937 | −0.3705401 | 0.36418601 |
| ITGB1 | | | | | | | | | |
| THBS2)- | 0.181532593 | −0.2830272 | −0.1684257 | 0.05086014 | 0.24585076 | −0.2382207 | −0.1081482 | 0.18311543 | −0.1990433 |
| ITGB3 | | | | | | | | | |
| THBS2)- | 0.021322818 | −0.0204672 | 0.06684159 | −0.0533088 | 0.11964629 | −0.158648 | 0.02834758 | 0.07217483 | 0.13078872 |
| NOTCH4 | | | | | | | | | |
| THBS2)- | 0.25176931 | −0.0136829 | 0.17495665 | 0.12263251 | −0.1524739 | −0.3018748 | 0.18116707 | 0.35375122 | −0.1240454 |
| ITGA6 | | | | | | | | | |
| THBS2)- | 0.090549533 | −0.0906686 | 0.02693262 | 0.07675858 | 0.00864022 | −0.2048482 | 0.02752166 | 0.08395188 | 0.06603012 |
| CD36 | | | | | | | | | |
| TGFB2)- | −0.129408507 | 0.14043938 | 0.0967888 | 0.03691595 | 0.22459829 | 0.09418293 | −0.0814553 | −0.2087524 | 0.23187956 |
| ENG | | | | | | | | | |
| C4BPA)- | −0.057998495 | 0.04643285 | 0.06439816 | −0.0948409 | −0.064385 | −0.0680426 | −0.1907754 | −0.0239116 | −0.135511 |
| BMPR2 | | | | | | | | | |
| C4BPA)- | −0.074225082 | 0.17202024 | 0.11559292 | 0.20625903 | −0.026757 | 0.11307577 | 0.0097849 | 0.30162115 | 0.16513194 |
| CD40 | | | | | | | | | |
| CFH)- | 0.01988152 | 0.06146619 | −0.2303253 | 0.07087814 | 0.09940909 | −0.0033764 | −0.3090143 | 0.01001573 | 0.04425925 |
| ITGAM | | | | | | | | | |
| PTGS2)- | 0.000115742 | −0.169955 | 0.03864138 | −0.0335833 | −0.0023582 | 0.1188804 | −0.1974451 | 0.10930337 | 0.2800389 |
| ALOX5 | | | | | | | | | |
| FASLG)- | 0.044861467 | 0.10617725 | 0.14953388 | 0.14689668 | −0.0756207 | −0.0922964 | 0.11846108 | 0.13334271 | 0.09262248 |
| TNFRSF6B | | | | | | | | | |
| XCL1)- | 0.024444607 | −0.0267987 | 0.12392785 | 0.28683649 | 0.1324813 | 0.18612953 | 0.11557374 | 0.02098912 | 0.43045917 |
| ADGRV1 | | | | | | | | | |
| CCN2)- | 0.033550795 | 0.01031417 | −0.1260588 | −0.120081 | 0.2149746 | −0.1378174 | −0.1400256 | 0.05556841 | 0.06731014 |
| ITGAM | | | | | | | | | |
| CCN2)- | 0.245413042 | 0.25774513 | −0.2036609 | −0.2067197 | 0.10357982 | −0.1277792 | −0.2541814 | 0.31728698 | 0.35312969 |
| ITGB2 | | | | | | | | | |
| APOA2)- | −0.024937165 | −0.1373877 | 0.01590122 | −0.1252185 | 0.17218564 | 0.12870992 | 0.34609396 | 0.31876043 | −0.4104694 |
| LDLR | | | | | | | | | |
| SEMA4A)- | 0.044434555 | 0.06726261 | 0.14134371 | 0.05394127 | −0.047925 | −0.0310603 | −0.0927443 | 0.07502549 | −0.1286727 |
| PLXNB2 | | | | | | | | | |
| EFNA1)- | 0.201279137 | 0.04912347 | −0.0048889 | −3.52E−05 | −0.0535008 | 0.25610601 | −0.0460757 | −0.3345184 | −0.0076133 |
| EPHB6 | | | | | | | | | |
| EFNA1)- | 0.118783572 | −0.0531411 | 0.14672357 | 0.05268716 | 0.10996825 | 0.07591398 | −0.1539431 | −0.0615808 | −0.0762446 |
| EPHB1 | | | | | | | | | |
| EFNA3)- | 0.120559501 | 0.10798451 | 0.08431559 | −0.0923567 | −0.0685728 | 0.15122938 | −0.0180581 | −0.1372151 | 0.12294673 |
| EPHB6 | | | | | | | | | |
| EFNA3)- | 0.084973885 | 0.23514265 | 0.18914478 | 0.16588909 | 0.02197092 | 0.12124148 | 0.16604038 | −0.0750256 | 0.23040611 |
| EPHB1 | | | | | | | | | |
| ADAM12)- | −0.153795675 | 0.01854133 | −0.0158725 | 0.0027684 | 0.12472408 | 0.30299399 | −0.1399964 | −0.2352773 | −0.1223531 |
| ITGB1 | | | | | | | | | |
| ADAM12)- | 0.143320854 | −0.0764196 | 0.08563452 | −0.0162442 | −0.0367097 | −0.1178954 | 0.09370981 | 0.13953066 | −0.4284762 |
| SDC4 | | | | | | | | | |
| S100A8)- | −0.236233275 | −0.2798914 | 0.3461622 | 0.33065236 | −0.2315529 | 0.27276589 | −0.3091289 | 0.31813062 | 0.40059641 |
| ITGB2 | | | | | | | | | |
| S100A8)- | −0.186991644 | −0.2263827 | 0.33920796 | 0.34415542 | −0.2651585 | 0.24213803 | −0.2009171 | 0.24426783 | 0.45443867 |
| TLR4 | | | | | | | | | |
| S100A8)- | −0.023160083 | −0.0202791 | 0.11707964 | 0.26373755 | −0.1558397 | 0.13192102 | −0.0026988 | 0.09416776 | 0.33575979 |
| CD36 | | | | | | | | | |
| S100A8)- | 0.050026894 | −0.0004161 | 0.04767022 | 0.18775285 | 0.01833523 | 0.07213526 | 0.11431354 | 0.04716337 | 0.26130616 |
| AGER | | | | | | | | | |
| S100A9)- | −0.176782778 | −0.1927056 | 0.37337166 | 0.35643741 | −0.2924391 | 0.4299729 | −0.2789303 | 0.26635186 | 0.27799259 |
| ITGB2 | | | | | | | | | |
| S100A9)- | −0.176560725 | −0.2617717 | 0.37076459 | 0.30436075 | −0.3504402 | 0.28254275 | −0.3056946 | 0.22643637 | 0.38126694 |
| TLR4 | | | | | | | | | |
| S100A9)- | −0.021146859 | −0.0681749 | 0.05067524 | 0.18294864 | −0.2225527 | 0.2480162 | −0.0436032 | 0.19204458 | 0.18586012 |
| CD36 | | | | | | | | | |
| S100A9)- | 0.094505298 | −0.0738838 | 0.14194713 | 0.1291139 | −0.0600118 | 0.13500487 | 0.05047382 | −0.0528328 | 0.20026452 |
| AGER | | | | | | | | | |
| WNT2B)- | 0.107657753 | 0.07004116 | 0.06934174 | 0.08211556 | 0.01724898 | 0.14057681 | 0.01236026 | 0.11095715 | 0.46820505 |
| FZD4 | | | | | | | | | |
| L1CAM)- | 0.089996743 | 0.24757014 | 0.2533138 | −0.046535 | 0.00287534 | 0.1912624 | 0.16133179 | 0.04396854 | 0.25686963 |
| FGFR2 | | | | | | | | | |
| L1CAM)- | −0.141886851 | 0.00642026 | 0.14471994 | 0.09151169 | −0.026542 | 0.21216451 | 0.11502097 | 0.06366613 | 0.15796458 |
| EPHB2 | | | | | | | | | |
| L1CAM)- | 0.07739254 | 0.15512889 | 0.03450708 | 0.17929859 | 0.1212416 | 0.02573344 | 0.29079298 | 0.10043974 | 0.45790565 |
| CNTN1 | | | | | | | | | |
| COL11A1)- | −0.207785056 | 0.05575394 | 0.09083253 | −0.0948975 | 0.05955785 | 0.45848648 | 0.05573327 | −0.313795 | 0.0441713 |
| ITGB1 | | | | | | | | | |
| COL11A1)- | 0.083881239 | 0.01306631 | 0.15576689 | −0.0428924 | −0.1111909 | −0.0661585 | 0.20109791 | 0.16499109 | −0.0766138 |
| DDR1 | | | | | | | | | |
| CD40LG)- | 0.128739253 | 0.24546784 | −0.0101365 | 0.11594027 | −0.0833536 | 0.17576622 | −0.0770213 | 0.25170054 | 0.37666313 |
| ITGAM | | | | | | | | | |
| CD40LG)- | 0.127928372 | 0.29042209 | 0.01284393 | 0.03166566 | −0.1506014 | 0.08396118 | −0.0077453 | 0.24479009 | 0.04327206 |
| CD40 | | | | | | | | | |
| CD40LG)- | 0.091946748 | 0.16995175 | −0.1001853 | 0.0926324 | −0.114089 | 0.05224242 | −0.2600976 | 0.23796153 | 0.26619114 |
| ITGB2 | | | | | | | | | |
| CD40LG)- | −0.000382759 | 0.05192508 | 0.21675449 | 0.00973251 | 0.0030898 | −0.0524422 | 0.00995273 | 0.06882034 | 0.09610115 |
| TRAF3 | | | | | | | | | |
| BMP5)- | 0.069885113 | −0.219533 | −0.0674945 | 0.00818741 | 0.01633452 | −0.1195909 | −0.2098852 | −0.0364857 | −0.2101982 |
| BMPR2 | | | | | | | | | |
| BMP5)- | 0.10863508 | 0.17583563 | 0.05166318 | 0.08950905 | 0.05769132 | 0.0331418 | 0.10196485 | 0.29430448 | 0.32183225 |
| BMPR1A | | | | | | | | | |
| BMP5)- | −0.184536818 | 0.22231049 | 0.32853183 | 0.09006182 | 0.06965338 | 0.1636804 | 0.08295627 | 0.06347779 | 0.4273344 |
| BMPR1B | | | | | | | | | |
| ITGB3BP)- | −0.018108882 | 0.26572575 | 0.15740505 | −0.0080622 | −0.104208 | 0.08374398 | 0.05344459 | 0.01135205 | 0.27685593 |
| ITGB3 | | | | | | | | | |
| GNAS)- | −0.139741126 | −0.2883257 | −0.0271279 | −0.2469718 | 0.01211199 | −0.2036844 | −0.0366975 | −0.1510319 | −0.2524584 |
| CRHR1 | | | | | | | | | |
| GNAS)- | −0.206493668 | −0.1438741 | −0.2183237 | −0.2668735 | −0.0271222 | −0.3359508 | 0.16144991 | −0.2414965 | −0.0944825 |
| GCGR | | | | | | | | | |
| GNAS)- | −0.023269036 | −0.243724 | 0.03111117 | −0.2817383 | 0.04796323 | −0.0967772 | −0.0188169 | 0.17075737 | 0.01795684 |
| ADCY7 | | | | | | | | | |
| RBP4)- | 0.020142774 | 0.13082694 | 0.11707479 | 0.13823222 | 0.12945513 | 0.11628442 | 0.09019649 | 0.15273953 | 0.25605603 |
| STRA6 | | | | | | | | | |
| COL5A1)- | −0.312907454 | 0.3435806 | 0.36639365 | −0.3013002 | −0.210094 | 0.64927534 | 0.39234744 | −0.3756337 | 0.40180388 |
| ITGB1 | | | | | | | | | |
| CEL)- | −0.147820069 | −0.0721826 | 0.01734905 | 0.03353255 | −0.0575709 | 0.01657878 | −0.2701008 | 0.28245016 | 0.35657718 |
| CXCR4 | | | | | | | | | |
| MMP9)- | 0.018200262 | −0.1103849 | 0.19152581 | 0.20422688 | −0.0536922 | 0.11006559 | −0.0504546 | 0.15247698 | 0.30578982 |
| ITGAM | | | | | | | | | |
| MMP9)- | 0.148867395 | −0.07862 | −0.0489562 | 0.0730134 | −0.1168547 | 0.02519792 | −0.0007242 | 0.06189697 | 0.23729985 |
| RECK | | | | | | | | | |
| MMP9)- | −0.06414568 | −0.1379726 | 0.29772127 | 0.12347523 | −0.3209477 | 0.04923351 | −0.2827819 | 0.01443281 | 0.09206271 |
| CD44 | | | | | | | | | |
| MMP9)- | −0.192123123 | −0.1956402 | 0.28088782 | 0.31228249 | −0.2451146 | 0.28150491 | −0.2865188 | 0.30641039 | 0.37822225 |
| ITGB2 | | | | | | | | | |
| MMP9)- | 0.026072105 | −0.1034823 | 0.12601913 | 0.23623455 | 0.06595532 | 0.10563333 | 0.17212113 | 0.1113029 | 0.24259317 |
| EPHB2 | | | | | | | | | |
| SPTAN1)- | 0.107555756 | 0.13893613 | 0.03906265 | −0.1728939 | 0.02883438 | 0.24436527 | 0.05754707 | 0.10123196 | 0.12947364 |
| PTPRA | | | | | | | | | |
| COL9A2)- | −0.070952952 | −0.0352139 | −0.0140762 | −0.1492017 | −0.050725 | −0.0920207 | −0.1173577 | −0.136804 | −0.2506954 |
| ITGB1 | | | | | | | | | |
| COL9A2)- | 0.14014025 | 0.17409548 | 0.04899648 | 0.19532824 | 0.10393173 | 0.12818492 | 0.02812442 | 0.02550536 | 0.45242301 |
| MAG | | | | | | | | | |
| PLAU)- | −0.027558023 | −0.0276581 | 0.02486137 | −0.0600475 | 0.13459143 | 0.06232002 | −0.0081334 | −0.0782335 | 0.01634861 |
| ITGAM | | | | | | | | | |
| PLAU)- | −0.119904969 | 0.22023494 | 0.26944433 | −0.0484733 | 0.07911203 | 0.48449513 | 0.18148637 | −0.3361172 | 0.41154156 |
| ITGB1 | | | | | | | | | |
| PLAU)- | 0.052616701 | −0.0069134 | 0.0408691 | −0.0608412 | 0.29968605 | −0.045163 | −0.0084045 | 0.06370121 | 0.12669333 |
| ITGB2 | | | | | | | | | |
| ANGPTL2)- | 0.026859501 | 0.01698759 | 0.00851286 | −0.0815825 | 0.16491648 | −0.016435 | 0.0077068 | 0.23944669 | 0.32916246 |
| LILRB2 | | | | | | | | | |
| GHRH)- | 0.207098169 | 0.260872 | 0.10174728 | 0.16074401 | 0.01733627 | −0.0027619 | 0.03276811 | 0.25944676 | 0.36350987 |
| GPR84 | | | | | | | | | |
| COL16A1)- | −0.269614571 | 0.17178151 | 0.21667018 | −0.1175911 | −0.0600647 | 0.39395568 | 0.13583263 | −0.3744004 | 0.17325203 |
| ITGB1 | | | | | | | | | |
| TNFSF15)- | −0.118096624 | −0.0478368 | 0.05649105 | 0.12485644 | −0.1040729 | −0.0143592 | −0.1718811 | −0.0075041 | 0.04192176 |
| TNFRSF25 | | | | | | | | | |
| HSPG2)- | 0.047362639 | −0.1311977 | 0.06552611 | 0.24713858 | 0.13804539 | 0.2938451 | 0.05740274 | −0.0471946 | −0.1152197 |
| SDC1 | | | | | | | | | |
| HSPG2)- | 0.00331024 | 0.21134084 | 0.19270085 | −0.1916845 | 0.04479234 | 0.58998768 | 0.04362345 | −0.0148958 | 0.23766791 |
| ITGB1 | | | | | | | | | |
| HSPG2)- | −0.109651931 | −0.1036301 | 0.07824483 | −0.1447917 | −0.1790506 | −0.0025607 | −0.0460366 | −0.0174024 | −0.1135612 |
| COL13A1 | | | | | | | | | |
| HSPG2)- | −0.062926922 | 0.07510359 | 0.23073487 | −0.3411512 | −0.1577036 | 0.35892549 | 0.10283886 | −0.0450031 | 0.01648036 |
| FGFR1 | | | | | | | | | |
| COL5A2)- | −0.345836293 | 0.46127212 | 0.41543635 | −0.3026815 | −0.1954229 | 0.70493513 | 0.42481018 | −0.3696256 | 0.53394283 |
| ITGB1 | | | | | | | | | |
| COL5A2)- | 0.284642154 | 0.03614505 | 0.32631468 | 0.24955547 | −0.3690573 | −0.3888275 | 0.4323854 | 0.45778848 | −0.0082402 |
| DDR1 | | | | | | | | | |
| F10)- | 0.188124669 | 0.18742355 | −0.0239322 | 0.09649797 | −0.0021864 | 0.15890722 | 0.00013201 | 0.12621755 | 0.2255877 |
| ITGAM | | | | | | | | | |
| F10)- | 0.007110644 | 0.03276782 | −0.057726 | 0.13920776 | −0.052538 | 0.10157651 | −0.1532958 | 0.17774269 | 0.28698561 |
| ITGB2 | | | | | | | | | |
| SYK)- LAT | 0.018711982 | −0.2306163 | 0.18884231 | 0.00858169 | −0.0572875 | −0.0060505 | −0.0713124 | −0.0302832 | −0.019196 |
| FGF14)- | 0.097007743 | 0.19925556 | 0.02362217 | 0.09803591 | −0.0249701 | 0.22038952 | 0.20043199 | 0.02363979 | 0.34463274 |
| FGFR2 | | | | | | | | | |
| FGF14)- | 0.051685681 | −0.0312735 | −0.1678401 | −0.0097318 | 0.02885032 | −0.079136 | −0.1105916 | −0.0743207 | 0.21293854 |
| FGFR1 | | | | | | | | | |
| HLA-C)- | −0.125285993 | −0.3039941 | 0.12779634 | 0.04757811 | −0.2007344 | 0.18783286 | −0.0517192 | 0.0752809 | 0.06803076 |
| LILRB2 | | | | | | | | | |
| HLA-C)- | 0.06462165 | −0.1673585 | −0.0184645 | 0.09011764 | 0.02310614 | 0.08695245 | 0.28287038 | −0.0954489 | 0.00328377 |
| CD3G | | | | | | | | | |
| HLA-C)- | −0.009177994 | −0.3447282 | 0.16812618 | −0.1080749 | 0.06583346 | 0.18402775 | 0.10526748 | 0.07200765 | 0.02063133 |
| KIR3DL1 | | | | | | | | | |
| HLA-E)- | −0.123553028 | −0.2484683 | 0.16055977 | −0.0491219 | 0.10950105 | −0.0595205 | 0.10444057 | −0.0188627 | 0.01086194 |
| KLRC2 | | | | | | | | | |
| HLA-E)- | −0.108268351 | −0.2534024 | 0.1910857 | −0.0681946 | 0.09384694 | 0.01526176 | 0.12324277 | −0.1410937 | −0.1461321 |
| KLRD1 | | | | | | | | | |
| HLA-E)- | −0.137811434 | −0.1787168 | 0.11754788 | −0.180113 | 0.23402211 | −0.0179045 | 0.21837161 | 0.02946096 | −0.158146 |
| KIR3DL1 | | | | | | | | | |
| ANXA1)- | −0.064740562 | −0.1229421 | 0.15749867 | −0.0884371 | 0.227761 | −0.1208362 | −0.2403376 | 0.21354761 | 0.14220683 |
| FPR1 | | | | | | | | | |
| ANXA1)- | −0.07038441 | −0.1005501 | −0.0055934 | −0.0365583 | 0.07605231 | −0.0542193 | −0.0250902 | 0.0701961 | 0.06042604 |
| DYSF | | | | | | | | | |
| IL31)- | 0.081852716 | 0.16327052 | 0.06897032 | 0.14476707 | −0.1093833 | 0.088076 | 0.11887421 | 0.31549449 | 0.37126013 |
| IL31RA | | | | | | | | | |
| ACTR2)- | −0.192886605 | 0.16708534 | −0.111996 | 0.16581422 | −0.0299645 | −0.0340964 | −0.2286016 | −0.1774234 | 0.12798535 |
| LDLR | | | | | | | | | |
| NDP)- | 0.058044553 | 0.10778159 | 0.18666524 | 0.21667626 | −0.0039448 | 0.15060833 | 0.00864166 | 0.26861815 | 0.46819617 |
| FZD4 | | | | | | | | | |
| NDP)- | 0.041964283 | −0.0644999 | 0.20932025 | −0.0641289 | −0.0552264 | 0.1074022 | 0.29267117 | 0.11165502 | −0.1590403 |
| LGR4 | | | | | | | | | |
| CCL7)- | 0.219804731 | 0.25928412 | 0.33081787 | 0.23608849 | 0.17957412 | 0.30078665 | 0.2467089 | 0.20708047 | 0.4796807 |
| CCR3 | | | | | | | | | |
| BMP2)- | 0.153175909 | 0.25007803 | 0.17780299 | 0.15490934 | −0.0238753 | 0.04676741 | 0.13837316 | −0.1364698 | 0.10542119 |
| BMPR1B | | | | | | | | | |
| AGRN)- | −0.170713166 | 0.14680052 | 0.17743582 | 0.12057352 | 0.18323281 | −0.1553804 | 0.00996558 | 0.19424293 | 0.21035549 |
| ITGB1 | | | | | | | | | |
| AGRN)- | −0.114822599 | 0.02850171 | −0.047432 | 0.03434437 | −0.1225181 | 0.06163058 | 0.00706287 | 0.05423548 | −0.0620232 |
| ATP1A3 | | | | | | | | | |
| INSL3)- | −0.005184804 | 0.06390608 | 0.16450606 | 0.16186664 | 0.01879946 | 0.21076973 | 0.02394329 | 0.12685852 | 0.48465976 |
| GPR84 | | | | | | | | | |
| INSL3)- | 0.109207828 | 0.25746416 | 0.23698479 | 0.04447811 | −0.0427561 | 0.1664405 | −0.0212192 | 0.11805095 | 0.34297381 |
| RXFP1 | | | | | | | | | |
| UCN3)- | −0.003973955 | −0.0242999 | 0.15750288 | 0.11192009 | −0.0434655 | 0.20321682 | 0.10068892 | 0.26074741 | 0.45692927 |
| CRHR1 | | | | | | | | | |
| SHBG)- | 0.212087722 | −0.1962394 | 0.21659287 | −0.0618108 | −0.1205714 | −0.1192998 | 0.3517672 | 0.31300906 | −0.4114224 |
| CLDN4 | | | | | | | | | |
| ZG16B)- | 0.118787485 | 0.18346732 | 0.15085147 | 0.20416575 | 0.08547867 | 0.14296596 | −0.2118484 | −0.1837367 | −0.280527 |
| TLR5 | | | | | | | | | |
| ZG16B)- | 0.053161617 | 0.14875643 | −0.1558909 | 0.22031284 | −0.2691116 | 0.09797411 | 0.04600351 | −0.0053958 | −0.0874261 |
| CXCR4 | | | | | | | | | |
| RPH3A)- | 0.184692066 | 0.26921377 | 0.16213221 | 0.13674891 | 0.05167484 | 0.11372523 | 0.12987113 | 0.13201989 | 0.39094659 |
| NRXN1 | | | | | | | | | |
| LAMA1)- | 0.076984063 | 0.05407853 | 0.02807813 | −0.1475823 | −0.0513247 | −0.0594852 | −0.0184987 | 0.12512065 | −0.0508445 |
| ITGA6 | | | | | | | | | |
| LAMA1)- | 0.023931833 | 0.07208723 | 0.34282573 | 0.23504954 | −0.1976729 | 0.10044899 | 0.14612682 | 0.01136815 | 0.43810665 |
| ITGA7 | | | | | | | | | |
| LAMA1)- | −0.160815669 | 0.02726346 | −0.0901747 | −0.1132202 | −0.0635611 | 0.1709533 | −0.0704476 | −0.2621283 | −0.108157 |
| ITGB1 | | | | | | | | | |
| LAMB3)- | 0.169452948 | 0.05740901 | 0.30258122 | 0.01598828 | 0.30993982 | 0.24296448 | −0.0743095 | −0.0566679 | 0.17020871 |
| CD151 | | | | | | | | | |
| LAMB3)- | −0.253649606 | 0.17032271 | 0.22097628 | 0.0981119 | 0.15409402 | −0.2370988 | 0.07380718 | 0.11941501 | 0.21374703 |
| ITGB1 | | | | | | | | | |
| LAMB3)- | 0.591933821 | −0.2961431 | 0.58296424 | −0.2787663 | 0.52709036 | 0.63909004 | −0.2538433 | −0.3027688 | 0.25551336 |
| ITGA6 | | | | | | | | | |
| AFDN)- | 0.217604612 | 0.23405221 | −0.0548033 | 0.21357294 | 0.09682476 | 0.14807051 | −0.0270463 | −0.1997059 | 0.05714545 |
| EPHB6 | | | | | | | | | |
| AFDN)- | −0.013762227 | 0.13927069 | 0.22395862 | 0.07315805 | 0.00677713 | −0.0254407 | −0.1095022 | −0.1076392 | −0.1768747 |
| NECTIN3 | | | | | | | | | |
| IL1F10)- | 0.05123283 | −0.0648768 | −0.1081538 | 0.07202116 | −0.1316867 | −0.0360808 | −0.1889933 | 0.08596186 | −0.037885 |
| IL1R1 | | | | | | | | | |
| CCL8)- | 0.026243087 | 0.31227894 | 0.07843966 | 0.16320318 | 0.21734206 | 0.23780465 | 0.22139922 | 0.36989207 | 0.59492609 |
| CCR3 | | | | | | | | | |
| SPP1)- | −0.08937122 | 0.0185202 | −0.0109589 | −0.0363736 | −0.0921895 | 0.0789777 | −0.0691851 | 0.00015233 | −0.0141486 |
| ITGA4 | | | | | | | | | |
| SPP1)- | −0.032841033 | −0.0992654 | 0.31019779 | 0.16137116 | −0.2647231 | 0.26852699 | −0.330813 | 0.20903698 | 0.25005859 |
| CD44 | | | | | | | | | |
| SPP1)- | 0.344689091 | 0.01790501 | 0.01393261 | 0.1064961 | 0.22042361 | −0.2030782 | 0.15768537 | −0.2796681 | 0.09008848 |
| ITGB1 | | | | | | | | | |
| SPP1)- | −0.100818154 | 0.02917784 | 0.07795772 | −0.0055336 | −0.0040242 | −0.0394458 | 0.040415 | −0.0669281 | 0.04619574 |
| ITGB3 | | | | | | | | | |
| ADM2)- | 0.060719784 | 0.1819117 | 0.02416029 | 0.04567255 | −0.122466 | 0.12017662 | −0.0278999 | −0.0138115 | 0.19852684 |
| CALCRL | | | | | | | | | |
| ADM2)- | 0.045818901 | 0.07593134 | −0.0830291 | 0.04127346 | 0.06183371 | 0.14233893 | −0.0249438 | 0.13920237 | −0.0174178 |
| GPR84 | | | | | | | | | |
| CXCL12)- | −0.168139969 | 0.1067068 | 0.15118447 | −0.2106894 | −0.1308441 | −0.0327223 | −0.1584943 | −0.1266743 | −0.262483 |
| ITGB1 | | | | | | | | | |
| CXCL12)- | 0.129727372 | 0.1736879 | −0.1341401 | 0.00133009 | −0.0471375 | 0.02872001 | −0.1890861 | 0.20381399 | 0.21864811 |
| CXCR4 | | | | | | | | | |
| BMP7)- | −0.059616017 | 0.21338573 | 0.33703867 | 0.21700748 | −0.0541414 | 0.30802831 | 0.05623489 | −0.1217324 | 0.2275367 |
| BMPR1B | | | | | | | | | |
| COL4A3)- | −0.04346685 | 0.22275303 | 0.14232938 | 0.1713547 | 0.10602725 | 0.17344119 | 0.09009688 | −0.0079598 | 0.37113764 |
| ITGB3 | | | | | | | | | |
| COL4A4)- | −0.127148512 | 0.08513614 | 0.21095307 | 0.15181478 | 0.07373613 | 0.11019974 | 0.09841987 | 0.02013394 | 0.3329159 |
| ITGB3 | | | | | | | | | |
| CNTN4)- | 0.050831937 | −0.0598056 | −0.0351932 | 0.11083489 | −0.0310848 | −0.003673 | 0.12235959 | −0.0003486 | 0.08609987 |
| PTPRG | | | | | | | | | |
| TCTN1)- | −0.105878787 | −0.0465461 | −0.0297195 | 0.03126996 | 0.0275187 | 0.26984327 | 0.07107672 | 0.02165592 | 0.18429031 |
| TMEM67 | | | | | | | | | |
| PDCD1LG2)- | 0.017176166 | 0.00931568 | 0.16374389 | 0.08498979 | 0.04730813 | 0.18994704 | 0.07110311 | 0.17666226 | 0.20161113 |
| PDCD2 | | | | | | | | | |
| NPS)- | 0.119512769 | 0.30012458 | 0.23722092 | 0.11132595 | 0.07676507 | 0.14933708 | −0.0287775 | 0.17335195 | 0.55155035 |
| GPR84 | | | | | | | | | |
| TNFSF11)- | −0.077519914 | 0.14910089 | 0.00750366 | −0.0555623 | 0.17448883 | 0.11939929 | 0.01716531 | 0.05057769 | 0.0106423 |
| TNFRSF11A | | | | | | | | | |
| POMC)- | 0.000156877 | 0.00535965 | 0.14613933 | 0.04904719 | −0.0051001 | 0.05656941 | 0.06875305 | 0.25312302 | 0.10766585 |
| MC1R | | | | | | | | | |
| POMC)- | 0.029163089 | 0.27141867 | 0.18225597 | 0.18355815 | −0.0715129 | 0.14041086 | 0.13465843 | 0.06131229 | 0.35417063 |
| GPR84 | | | | | | | | | |
| FBLN2)- | 0.109110076 | 0.02721411 | 0.07131539 | 0.12490863 | 0.09104362 | −0.150339 | 0.04892126 | 0.08799973 | 0.19161417 |
| ITGB3 | | | | | | | | | |
| MDK)- | 0.239349689 | −0.3423703 | 0.26754156 | −0.1458961 | 0.13548503 | 0.15209734 | −0.0241494 | −0.009904 | 0.40412596 |
| ITGA6 | | | | | | | | | |
| GCG)- | −0.056140819 | −0.1056608 | −0.0326126 | 0.26695074 | 0.06649623 | 0.06910014 | 0.15558111 | 0.12696362 | 0.40333417 |
| GPR84 | | | | | | | | | |
| PTPN6)- | −0.018658866 | −0.1816193 | 0.21402512 | 0.31902643 | −0.093799 | 0.19366578 | 0.03383263 | 0.02039887 | 0.17369563 |
| CD300LF | | | | | | | | | |
| APLN)- | −0.04001073 | 0.05445289 | 0.06775743 | 0.05856477 | 0.02889979 | 0.08147729 | 0.1696807 | 0.01785246 | 0.38135175 |
| APLNR | | | | | | | | | |
| CCN1)- | 0.094937896 | 0.06006058 | −0.1037322 | −0.1195314 | 0.20839534 | −0.1004648 | −0.0799107 | 0.14667963 | 0.14747419 |
| ITGAM | | | | | | | | | |
| CCN1)- | 0.111176575 | −0.0714883 | −0.1322121 | −0.0620179 | 0.26247644 | −0.245372 | −0.0923096 | 0.17549831 | 0.02972404 |
| ITGB3 | | | | | | | | | |
| HLA-B)- | −0.019508029 | −0.1829556 | −0.0552785 | −0.0897985 | 0.13500492 | −0.0272088 | 0.39423501 | −0.145454 | −0.1493368 |
| CD3G | | | | | | | | | |
| LAMA2)- | 0.131443148 | 0.1118193 | −0.1260245 | 0.03396938 | −0.045814 | −0.0983046 | 0.05683719 | 0.10794435 | 0.33984085 |
| ITGA7 | | | | | | | | | |
| CCL24)- | 0.166723335 | 0.20304212 | 0.25222434 | 0.17387216 | 0.13845469 | 0.20873496 | 0.21277469 | 0.07996848 | 0.33604003 |
| CCR3 | | | | | | | | | |
| HRAS)- | 0.061509169 | 0.12008144 | 0.15065762 | 0.05651319 | 0.07034316 | 0.100181 | 0.11522851 | −0.0810078 | 0.08534335 |
| AGTR1 | | | | | | | | | |
| ICAM2)- | 0.021617053 | −0.0408637 | −0.046699 | 0.03517347 | −0.0068753 | 0.08943265 | 0.04787055 | 0.11331292 | 0.34806311 |
| ITGAM | | | | | | | | | |
| FSHB)- | −0.084635822 | 0.15202627 | 0.28207291 | 0.31184732 | −0.0345287 | 0.20133017 | 0.04928979 | 0.20575858 | 0.45852215 |
| GPR84 | | | | | | | | | |
| RARRES2)- | 0.07701785 | −0.0501074 | −0.1221436 | −0.0789794 | 0.10991379 | −0.2347942 | −0.0127182 | 0.11998096 | 0.18968876 |
| GPR1 | | | | | | | | | |
| LRPAP1)- | 0.051878217 | −0.1491807 | 0.05589558 | −0.2131673 | 0.09735911 | 0.05849574 | 0.15662271 | 0.11920926 | −0.2176879 |
| LDLR | | | | | | | | | |
| HSPA8)- | −0.047669056 | 0.17557869 | −0.0958244 | 0.09438691 | 0.03913326 | 0.06774867 | −0.0644044 | −0.0698061 | 0.12044872 |
| LDLR | | | | | | | | | |
| CALCB)- | 0.107268254 | 0.15207158 | 0.02688583 | 0.16964716 | 0.15509377 | 0.10462343 | 0.15333241 | 0.16900162 | 0.4622255 |
| GPR84 | | | | | | | | | |
| ADM)- | −0.01598019 | 0.2004202 | 0.14962992 | 0.10032353 | 0.04158081 | 0.04942037 | 0.09375096 | 0.05100561 | 0.15000491 |
| GPR84 | | | | | | | | | |
| PTHLH)- | −0.035842851 | 0.23874331 | 0.25091797 | 0.13311786 | −0.0756565 | 0.1584697 | 0.08248439 | 0.16580412 | 0.35486299 |
| GPR84 | | | | | | | | | |
| S100A10)- | 0.087195881 | 0.08066191 | 0.23017113 | 0.0739186 | 0.24504809 | 0.04502039 | −0.0311807 | −0.3179525 | −0.0840131 |
| CFTR | | | | | | | | | |
| SHANK2)- | 0.014063635 | −0.0361858 | 0.13692365 | 0.0196613 | −0.0161975 | 0.11225228 | −0.0033399 | 0.1060237 | 0.07664907 |
| CFTR | | | | | | | | | |
| HSP90AA1)- | −0.108557568 | 0.02912043 | 0.20756856 | 0.04409514 | 0.20199043 | −0.1707382 | 0.00422169 | −0.2096794 | −0.1106203 |
| CFTR | | | | | | | | | |
| PLAU)- | 0.060114452 | 0.03798861 | 0.06881051 | 0.1161048 | −0.1271724 | −0.1017536 | 0.11559429 | 0.06639317 | 0.33777175 |
| ITGA3 | | | | | | | | | |
| LAMA3)- | 0.489196663 | 0.07282879 | 0.58317893 | −0.1485049 | 0.55950998 | 0.5808727 | −0.2405756 | −0.2825322 | 0.35782508 |
| ITGA3 | | | | | | | | | |
| LAMA4)- | 0.084490744 | −0.1453933 | 0.12164716 | 0.14463804 | −0.2382087 | −0.2013784 | 0.33472574 | 0.23346725 | 0.15070209 |
| ITGA3 | | | | | | | | | |
| LAMA2)- | 0.118705396 | 0.03474411 | 0.09613314 | 0.06525724 | −0.2199098 | −0.2019607 | 0.24147931 | 0.19666028 | −0.1425088 |
| ITGA3 | | | | | | | | | |
| LAMA1)- | 0.064429352 | −0.0419956 | 0.12252584 | 0.06986094 | −0.0937712 | −0.067678 | 0.14983859 | 0.11098076 | −0.1944463 |
| ITGA3 | | | | | | | | | |
| LGALS8)- | 0.104956646 | 0.16410907 | 0.10080929 | 0.10846406 | −0.0967385 | −0.0582387 | 0.05254963 | 6.74E−05 | −0.1312845 |
| ITGA3 | | | | | | | | | |
| NID1)- | 0.27528972 | 0.01922581 | 0.28773727 | −0.0423752 | −0.4219683 | −0.3890021 | 0.48518198 | 0.42163834 | −0.1046238 |
| ITGA3 | | | | | | | | | |
| LAMC3)- | −0.021067138 | 0.04809065 | 0.04455265 | 0.03993496 | −0.1073616 | −0.0607785 | 0.16395143 | 0.10677376 | 0.08614548 |
| ITGA3 | | | | | | | | | |
| THBS1)- | 0.332733797 | −0.0735203 | 0.33316636 | −0.096263 | −0.2656705 | −0.2154836 | 0.40006972 | 0.33850066 | 0.1345906 |
| ITGA3 | | | | | | | | | |
| CALR)- | −0.073020583 | −0.2724643 | −0.0240854 | −0.1968284 | −0.1380089 | −0.0415 | 0.17580055 | 0.18651575 | 0.38032551 |
| ITGA3 | | | | | | | | | |
| TIMP2)- | 0.235175394 | −0.0594097 | 0.26426403 | 0.10604576 | −0.4917026 | −0.4384578 | 0.39044395 | 0.37140279 | −5.86E−05 |
| ITGA3 | | | | | | | | | |
| LAMB2)- | 0.108745992 | −0.035779 | 0.13360752 | 0.01517294 | −0.2075967 | −0.1778146 | 0.08400732 | 0.0537707 | 0.18257608 |
| ITGA3 | | | | | | | | | |
| COL4A3)- | 0.015908825 | 0.03715577 | 0.02817393 | 0.08259233 | −0.0729034 | −0.135394 | 0.11079198 | 0.06657791 | −0.0323223 |
| ITGA3 | | | | | | | | | |
| LAMA5)- | 0.405703615 | −0.1263642 | 0.37918983 | −0.0918717 | 0.34907885 | 0.43025478 | 0.04423308 | −0.0433191 | 0.53112422 |
| ITGA3 | | | | | | | | | |
| ADAM9)- | 0.410449521 | −0.0922213 | 0.49094834 | −0.0299379 | 0.47531293 | 0.52664311 | −0.1814467 | −0.212931 | 0.33848556 |
| ITGA3 | | | | | | | | | |
| LAMC1)- | 0.13696062 | −0.0506978 | 0.18877774 | 0.01266383 | −0.3504865 | −0.3068173 | 0.36869664 | 0.34799166 | 0.16781496 |
| ITGA3 | | | | | | | | | |
| LAMB3)- | 0.570262702 | 0.06637256 | 0.64480609 | −0.0130648 | 0.62014995 | 0.64532363 | −0.1112148 | −0.1602925 | 0.41524302 |
| ITGA3 | | | | | | | | | |
| COL18A1)- | 0.221032866 | −0.0348341 | 0.24419638 | 0.23385409 | −0.0823612 | −0.0732922 | 0.48392752 | 0.44422572 | −0.099427 |
| ITGA3 | | | | | | | | | |
| VTN)- | −0.082069592 | 0.01435609 | −0.0229446 | 0.09938008 | −0.0459167 | −0.0252566 | 0.05192538 | 0.06549396 | −0.0718272 |
| ITGA3 | | | | | | | | | |
| LAMB1)- | 0.318220366 | −0.1914736 | 0.35295996 | 0.32549133 | −0.2637107 | −0.2264454 | 0.49637716 | 0.42732354 | −0.0396044 |
| ITGA3 | | | | | | | | | |
| LAMC2)- | 0.542352128 | −0.0574674 | 0.6126128 | −0.1259315 | 0.62529971 | 0.65923205 | −0.1607438 | −0.1964813 | 0.38686961 |
| ITGA3 | | | | | | | | | |
| FN1)- | 0.209803853 | −0.2705521 | 0.27702632 | 0.15479549 | −0.2315623 | −0.24429 | 0.43087744 | 0.37237533 | −0.2180294 |
| ITGA3 | | | | | | | | | |
| MIF)- CD74 | 0.04648499 | 0.03962415 | 0.1316148 | 0.07240182 | −0.0450543 | 0.09984034 | −0.0720856 | 0.10651969 | 0.13163881 |
| APP)- CD74 | 0.161747065 | 0.14010795 | 0.03908651 | −0.0400967 | 0.24062208 | −0.0669381 | 0.02999931 | −0.1528578 | −0.1758765 |
| HLA-G)- | −0.159476692 | −0.151488 | 0.21733042 | 0.1800068 | 0.00762281 | 0.2302157 | −0.0074558 | 0.12304046 | 0.0698233 |
| CD4 | | | | | | | | | |
| CXCL12)- | 0.030688004 | 0.0992988 | −0.1596636 | 0.04085196 | 0.09349088 | 0.09103374 | −0.0016046 | 0.0832136 | 0.2357349 |
| CD4 | | | | | | | | | |
| SLPI)- CD4 | 0.14651608 | 0.32824803 | −0.2707136 | 0.29659889 | −0.3627263 | 0.14646735 | 0.07745614 | −0.1591798 | −0.21752 |
| ADCYAP1)- | 0.048838676 | −0.1521683 | −0.0181966 | −0.0353861 | 0.08736113 | 0.25289406 | 0.02893492 | 0.19732916 | 0.05711967 |
| SCTR | | | | | | | | | |
| SCT)- | 0.195073106 | −0.0392133 | 0.21585138 | −0.0347463 | −0.0569271 | 0.10232045 | 0.203157 | 0.11460486 | 0.1112859 |
| SCTR | | | | | | | | | |
| CALM1)- | −0.062571139 | 0.18036216 | −0.0430993 | 0.14957375 | −0.0954546 | −0.0400855 | −0.1741326 | −0.0086479 | 0.03678669 |
| SCTR | | | | | | | | | |
| RTN4)- | −0.133445963 | −0.1500364 | −0.0033084 | 0.03839354 | 0.07406643 | −0.1595206 | −0.0552868 | −0.072475 | −0.1602549 |
| RTN4R | | | | | | | | | |
| TNFSF13B)- | 0.126147816 | 0.03238678 | 0.11717579 | 0.27786965 | −0.1015838 | 0.21147726 | 0.02014813 | −0.0398995 | 0.23456678 |
| TNFRSF17 | | | | | | | | | |
| ST6GAL1)- | −0.086431773 | −0.1235644 | 0.06337678 | 0.12552766 | 0.01691898 | 0.21506769 | −0.0663414 | 0.00399148 | 0.02979732 |
| CD22 | | | | | | | | | |
| CALM2)- | −0.067940003 | −0.0272919 | 0.01382381 | −0.021717 | 0.05422651 | −0.0678655 | −0.0033108 | 0.03593084 | −0.0674475 |
| KCNQ1 | | | | | | | | | |
| CALML3)- | 0.044240747 | −0.0543846 | 0.04685754 | −0.2267902 | −0.0329264 | 0.26506984 | −0.0089588 | 0.06548035 | 0.19539354 |
| KCNQ1 | | | | | | | | | |
| CALM1)- | −0.178028928 | 0.02819101 | 0.20034558 | 0.02166859 | 0.08990055 | −0.0841467 | −0.1105099 | 0.02644821 | 0.17796553 |
| KCNQ1 | | | | | | | | | |
| CALM3)- | 0.124669637 | −0.0893216 | −0.0896969 | −0.0549406 | −0.0174726 | 0.0187507 | 0.11673426 | −0.1861632 | 0.08964791 |
| KCNQ1 | | | | | | | | | |
| FASLG)- | −0.152615907 | −0.0765827 | 0.0442741 | 0.006118 | −0.0310215 | −0.0106204 | −0.1169183 | −0.0622529 | 0.06440122 |
| TNFRSF1A | | | | | | | | | |
| LTA)- | −0.162936995 | 0.00680063 | −0.0281398 | −0.0815901 | −0.1438119 | −0.0573913 | −0.1919066 | 0.00786986 | 0.04062096 |
| TNFRSF1A | | | | | | | | | |
| TNF)- | −0.16569744 | 0.0024439 | 0.01287914 | −0.0137408 | −0.1308197 | −0.0294909 | −0.1886474 | −0.1252292 | −0.0226202 |
| TNFRSF1A | | | | | | | | | |
| LTB)- | −0.109602235 | −0.0686128 | −0.0687948 | 0.08738486 | −0.108172 | 0.0304523 | −0.0622586 | 0.07983525 | −0.0766429 |
| TNFRSF1A | | | | | | | | | |
| IL2)- NGFR | 0.095588303 | 0.16966499 | −0.0018409 | 0.12022762 | −5.72E−05 | 0.07300721 | 0.02016112 | 0.04658713 | 0.35299115 |
| RTN4)- | −0.124557019 | −0.2311939 | −0.034656 | −0.258992 | −0.0680671 | −0.0024187 | 0.0522647 | 0.01719612 | 0.07837106 |
| NGFR | | | | | | | | | |
| NTF4)- | 0.135764462 | 0.13571292 | 0.07309842 | 0.18435565 | 0.05103844 | 0.00599172 | 0.00544269 | 0.08940468 | 0.45204506 |
| NGFR | | | | | | | | | |
| APP)- | −0.110205412 | −0.1362061 | 0.07746711 | −0.0278931 | 0.02172939 | −0.2184124 | 0.03562876 | 0.02918564 | −0.3693113 |
| NGFR | | | | | | | | | |
| BDNF)- | −0.01642288 | 0.11683314 | 0.08062551 | 0.25081941 | 0.03080111 | 0.1812096 | 0.14936289 | 0.03427503 | 0.45828646 |
| NGFR | | | | | | | | | |
| BDNF)- | −0.061610979 | 0.17111465 | 0.1433353 | 0.12512876 | 0.09069722 | 0.14839126 | 0.018823 | 0.15503916 | 0.37552838 |
| DRD4 | | | | | | | | | |
| NPY)- FAP | 0.125363193 | 0.08500845 | 0.03883916 | 0.16383192 | 0.11767561 | 0.03912724 | 0.09490704 | 0.06216705 | 0.2331564 |
| CD99)- | 0.082406379 | 0.11702997 | −0.1223914 | −0.2833682 | 0.23661488 | −0.1758398 | −0.1991328 | 0.3524387 | 0.39432766 |
| PILRA | | | | | | | | | |
| LAMA3)- | 0.509649184 | 0.09159402 | 0.57124109 | 0.02202264 | 0.55037261 | 0.57081927 | −0.2137981 | −0.2544691 | 0.051284 |
| ITGB4 | | | | | | | | | |
| LAMA2)- | 0.016494592 | −0.0412541 | 0.01516155 | −0.0646887 | −0.147713 | −0.1196222 | 0.14162704 | 0.12571787 | −0.0950566 |
| ITGB4 | | | | | | | | | |
| LAMC3)- | −0.082275281 | 0.07583525 | −0.0413223 | −0.1505897 | −0.0730725 | −0.1114165 | 0.15398271 | 0.15341442 | 0.03264436 |
| ITGB4 | | | | | | | | | |
| LAMB2)- | 0.077059291 | 0.14486772 | 0.10742193 | 0.01376348 | −0.1462468 | −0.1337346 | 0.08298487 | 0.0381281 | 0.28917131 |
| ITGB4 | | | | | | | | | |
| LAMA5)- | 0.424592625 | 0.09656011 | 0.40862426 | 0.10165291 | 0.31310245 | 0.32468527 | 0.03137907 | −0.0123442 | 0.32607053 |
| ITGB4 | | | | | | | | | |
| LAMC1)- | 0.158948138 | 0.01641301 | 0.21972872 | 0.09987521 | −0.2737912 | −0.2647598 | 0.30423346 | 0.33544244 | 0.35742407 |
| ITGB4 | | | | | | | | | |
| LAMA1)- | 0.063910634 | −0.0214608 | 0.02867597 | 0.0396158 | −0.1964205 | −0.247924 | 0.12370578 | 0.12786837 | −0.0822909 |
| ITGB4 | | | | | | | | | |
| LAMB3)- | 0.61792276 | 0.08911581 | 0.70392178 | 0.1035105 | 0.66395412 | 0.70238095 | −0.1040788 | −0.1460794 | 0.13355362 |
| ITGB4 | | | | | | | | | |
| LAMB1)- | 0.243850211 | 0.0051615 | 0.30245171 | 0.31850179 | −0.1485703 | −0.1266496 | 0.41052095 | 0.36895388 | 0.19369259 |
| ITGB4 | | | | | | | | | |
| LAMC2)- | 0.684359768 | 0.08597558 | 0.75198022 | 0.122223 | 0.70252742 | 0.74180376 | −0.1435895 | −0.1278473 | 0.19903567 |
| ITGB4 | | | | | | | | | |
| TGFB1)- | −0.240851854 | −0.2685177 | 0.06075989 | 0.05585941 | 0.05161148 | 0.01516554 | −0.0266189 | −0.1529948 | −0.3106667 |
| TGFBR3 | | | | | | | | | |
| TGFB3)- | 0.010964222 | −0.1299077 | 0.03639605 | 0.01568942 | −0.0770376 | 0.1405832 | 0.05782132 | 0.05525975 | 0.04724416 |
| TGFBR3 | | | | | | | | | |
| INHA)- | −0.031076036 | 0.10543798 | 0.03158799 | 0.18082663 | 0.02171067 | 0.19825293 | −0.0684682 | 0.08963953 | 0.33675333 |
| TGFBR3 | | | | | | | | | |
| TGFB2)- | 0.066281104 | 0.05336568 | 0.0105113 | 0.06873292 | −0.1491071 | 0.10291106 | −0.03483 | 0.0289467 | −0.0950947 |
| TGFBR3 | | | | | | | | | |
| INHBA)- | 0.003272287 | −0.1109101 | −0.0382318 | −0.0881827 | 0.10058694 | −0.1567719 | −0.0482641 | −0.1181568 | −0.0121303 |
| TGFBR3 | | | | | | | | | |
| IL2)- IL2RB | 0.057038369 | 0.12396757 | 0.10450894 | 0.15940999 | −0.0103908 | 0.17568005 | 0.04108748 | 0.12375856 | 0.36774623 |
| KNG1)- | 0.034966553 | 0.05624455 | 0.22697046 | 0.2607604 | 0.14576139 | 0.09511084 | 0.18182992 | 0.16634098 | 0.37598701 |
| BDKRB1 | | | | | | | | | |
| PROC)- | 0.142855258 | 0.08844685 | 0.11665566 | 0.19611644 | −0.03031 | 0.17004165 | 0.06927819 | 0.20693042 | 0.15738503 |
| PROCR | | | | | | | | | |
| PF4)- | 0.001672535 | −0.0019694 | 0.13924876 | 0.01972376 | 0.08409908 | 0.34753889 | 0.17555054 | −0.0158241 | 0.07360831 |
| PROCR | | | | | | | | | |
| TNFSF10)- | 0.062430601 | 0.09545488 | 0.11913309 | 0.1044432 | −0.0318827 | 0.01583864 | 0.18006781 | 0.18572521 | −0.2023728 |
| TNFRSF10A | | | | | | | | | |
| APP)- | 0.04861537 | −0.2581755 | 0.02250537 | −0.0398159 | −0.0510998 | 0.01967988 | −0.0935257 | −0.0113741 | −0.4113518 |
| APLP1 | | | | | | | | | |
| CXCL8)- | −0.04899838 | 0.07495437 | −0.1325479 | 0.0926032 | 0.07826411 | 0.02020939 | 0.03747427 | 0.06737429 | 0.07632054 |
| CD79A | | | | | | | | | |
| FN1)- | 0.026358502 | 0.01329326 | −0.0227284 | −0.0494532 | −0.1376403 | −0.1080981 | −0.1363116 | 0.02124032 | 0.09360939 |
| CD79A | | | | | | | | | |
| KITLG)- | 0.057715729 | 0.06025018 | −0.0192647 | 0.00091402 | 0.11065177 | −0.1216099 | −0.0430629 | 0.15941135 | 0.16266198 |
| EPOR | | | | | | | | | |
| LAMA1)- | −0.00064335 | 0.06729011 | 0.06297864 | 0.10975004 | −0.0524213 | −0.0095052 | 0.09603897 | 0.14408888 | −0.1337251 |
| ITGB8 | | | | | | | | | |
| VTN)- | 0.014187834 | 0.1194444 | 0.11597538 | 0.04217576 | −0.2747409 | −0.059071 | −0.0417956 | −0.038508 | 0.1063999 |
| ITGB8 | | | | | | | | | |
| FN1)- | 0.177454536 | −0.2450776 | 0.21920156 | 0.23078151 | −0.1042934 | −0.0926846 | 0.24264067 | 0.29728784 | −0.2409443 |
| ITGB8 | | | | | | | | | |
| APP)- | −0.069598094 | −0.1376755 | −0.0561686 | −0.0311726 | −0.0420975 | 0.02195328 | −0.0988784 | −0.4233455 | −0.3395943 |
| TSPAN12 | | | | | | | | | |
| ADAM10)- | 0.161981859 | 0.23553184 | 0.00176444 | 0.21673016 | −0.2574283 | 0.14654868 | 0.12512642 | −0.2128013 | −0.0557862 |
| TSPAN12 | | | | | | | | | |
| C1QB)- | 0.125127996 | −0.0115867 | 0.08480243 | 0.07771333 | −0.0252085 | 0.15990918 | 0.05993033 | 0.10258679 | 0.14774585 |
| C1QBP | | | | | | | | | |
| GNRH1)- | 0.153207972 | 0.04476827 | 0.08889241 | 0.19943976 | 0.10882288 | 0.14652819 | 0.13016321 | 0.24037226 | 0.37482058 |
| GNRHR | | | | | | | | | |
| HBEGF)- | 0.026846493 | 0.06656999 | 0.14976387 | −0.0264404 | 0.02078564 | 0.06054001 | −0.0126373 | 0.05131165 | 0.03059974 |
| CD82 | | | | | | | | | |
| IL10)- | 0.011335317 | 0.02781953 | 0.00258941 | 0.13218561 | 0.05429225 | 0.06870347 | −0.1401501 | 0.11912487 | 0.37225339 |
| IL10RA | | | | | | | | | |
| S100A8)- | −0.004464914 | −0.0171961 | 0.15536874 | 0.28858478 | −0.1010967 | 0.26089262 | −0.1419789 | 0.15172879 | 0.40074288 |
| CD69 | | | | | | | | | |
| TNFSF14)- | −0.074015734 | 0.10976888 | −0.1271042 | −0.087975 | −0.04899 | −0.1733738 | −0.2375734 | −0.1626131 | −0.0684702 |
| LTBR | | | | | | | | | |
| LTA)- | −0.103047762 | −0.1229978 | −0.1538592 | 0.05071261 | −0.0692871 | −0.0645398 | −0.2110123 | −0.1900673 | −0.0790626 |
| LTBR | | | | | | | | | |
| LTB)- | −0.045759384 | −0.0941236 | −0.0358768 | 0.01366288 | −0.0405857 | −0.0655693 | −0.1429208 | −0.179214 | −0.017743 |
| LTBR | | | | | | | | | |
| CLEC2D)- | −0.068452191 | 0.18422769 | 0.23174703 | 0.14986061 | −0.0067365 | 0.23206404 | −0.0254467 | 0.15949359 | 0.37147071 |
| KLRB1 | | | | | | | | | |
| IL33)- | −0.057896985 | 0.01420447 | 0.07927344 | 0.14452415 | 0.03113113 | 0.2779465 | 0.10743575 | 0.01619266 | 0.1885975 |
| IL1RL1 | | | | | | | | | |
| GNAI2)- | −0.141484102 | −0.2927853 | −0.2564327 | −0.230758 | −0.0726014 | −0.1263227 | −0.0701272 | −0.1364883 | −0.1713176 |
| OPRD1 | | | | | | | | | |
| POMC)- | 0.240517784 | 0.29904588 | 0.15405104 | 0.12126976 | 0.1264458 | 0.12409391 | 0.1370428 | 0.12887014 | 0.38599976 |
| OPRD1 | | | | | | | | | |
| CXCL13)- | 0.096731725 | 0.2532 | 0.29754108 | 0.02652769 | −0.0920453 | 0.22812083 | 0.15887077 | 0.24101978 | 0.29435898 |
| OPRD1 | | | | | | | | | |
| PENK)- | 0.178284991 | 0.05751667 | 0.1608798 | 0.20126391 | 0.00916341 | 0.39002652 | 0.27879054 | 0.21864522 | 0.35310247 |
| OPRD1 | | | | | | | | | |
| CALM2)- | −0.044348242 | 0.06733065 | −0.0363165 | −0.062928 | −0.0758526 | −0.082252 | 0.25202152 | −0.0819046 | −0.1194551 |
| SELL | | | | | | | | | |
| PODXL2)- | −0.10306928 | 0.01034794 | 0.27696846 | 0.1921277 | −0.0610325 | 0.27379614 | −0.2011246 | 0.00825843 | −0.1689008 |
| SELL | | | | | | | | | |
| VCAN)- | 0.165373532 | 0.04985419 | −0.1343134 | −0.0809204 | −0.0395288 | −0.1276854 | −0.2259023 | 0.20813507 | 0.11877244 |
| SELL | | | | | | | | | |
| CD34)- | −0.068645037 | 0.12733187 | 0.13148047 | 0.06532309 | 0.1413838 | 0.06237331 | 0.17152979 | 0.03993114 | 0.22249432 |
| SELL | | | | | | | | | |
| MUC7)- | 0.010001027 | 0.0853636 | 0.1031903 | 0.19662489 | −0.0031117 | 0.19085361 | 0.12078851 | 0.18962808 | 0.32625361 |
| SELL | | | | | | | | | |
| CALM1)- | −0.083724782 | −0.0112468 | 0.0446206 | −0.0727696 | 0.09832189 | −0.1561518 | −0.1522513 | 0.13636304 | 0.05943317 |
| SELL | | | | | | | | | |
| PODXL)- | 0.069885267 | 0.08347639 | −0.0037223 | 0.05370132 | 0.13482716 | −0.0873489 | 0.05577081 | −0.0398931 | 0.02144562 |
| SELL | | | | | | | | | |
| CALM3)- | −0.085989353 | −0.0405661 | 0.0880857 | −0.0968976 | 0.18588373 | −0.1940866 | 0.01263011 | −0.0738675 | −0.1665151 |
| SELL | | | | | | | | | |
| CFH)- | −0.072922253 | −0.048451 | 0.03518789 | −0.0017518 | 0.22481303 | −0.0789324 | −0.2861116 | 0.02773318 | 0.11082225 |
| SELL | | | | | | | | | |
| HLA-E)- | −0.057964773 | −0.0654619 | 0.04461685 | 0.0154551 | −0.0483514 | −0.0046172 | 0.06112785 | 0.02403832 | −0.1067796 |
| KLRK1 | | | | | | | | | |
| MICB)- | −0.090301974 | −0.0121681 | 0.28061095 | 0.23944679 | 0.17750078 | −0.0331226 | 0.3180553 | −0.0527818 | 0.24731088 |
| KLRK1 | | | | | | | | | |
| MICA)- | 0.029233023 | 0.05364917 | 0.16113206 | −0.032082 | 0.18754268 | −0.0435055 | 0.07893153 | −0.0208962 | 0.2379667 |
| KLRK1 | | | | | | | | | |
| RAET1L)- | −0.05657007 | 0.08647159 | 0.19667878 | 0.15211267 | 0.04688528 | 0.13756867 | 0.25060039 | 0.02752659 | 0.18206318 |
| KLRK1 | | | | | | | | | |
| ULBP2)- | −0.015433005 | −0.019666 | 0.13371292 | 0.06614806 | 0.23768726 | −0.0131458 | 0.27189666 | −0.0204363 | 0.22450092 |
| KLRK1 | | | | | | | | | |
| RAET1E)- | 0.018808906 | 0.06740586 | 0.15460739 | 0.08734724 | 0.04704413 | 0.22573825 | 0.22601939 | 0.0616241 | 0.2685973 |
| KLRK1 | | | | | | | | | |
| RAET1G)- | 0.020987287 | 0.08398181 | 0.086024 | 0.0663134 | 0.06829257 | 0.0766493 | 0.11982764 | 0.07716951 | 0.27781968 |
| KLRK1 | | | | | | | | | |
| BMP7)- | 0.086523183 | 0.00939709 | 0.00392199 | 0.12361595 | 0.11989689 | −0.1304436 | 0.04785599 | 0.02071216 | 0.31231591 |
| ACVR2A | | | | | | | | | |
| LEFTY1)- | −0.040546627 | −0.0559249 | 0.09123239 | 0.10219703 | −0.050261 | 0.10366983 | −0.0078417 | 0.06662615 | 0.02160667 |
| ACVR2A | | | | | | | | | |
| INHA)- | −0.005609849 | 0.13811948 | 0.07905765 | 0.15637849 | 0.05288732 | 0.15643699 | 0.04909268 | 0.05371881 | 0.25862761 |
| ACVR2A | | | | | | | | | |
| INHBA)- | −0.198599449 | 0.18020599 | 0.02451257 | −0.2576891 | −0.0005407 | 0.02876006 | 0.02529791 | −0.1215974 | 0.2966348 |
| ACVR2A | | | | | | | | | |
| INHBB)- | 0.021668652 | 0.14139958 | 0.03993694 | 0.18082754 | 0.04387074 | 0.10912001 | 0.04092003 | 0.11928962 | 0.21572852 |
| ACVR2A | | | | | | | | | |
| TDGF1)- | 0.04569055 | 0.00120193 | 0.12429087 | 0.10878208 | 0.11667236 | 0.09584303 | 0.10127136 | 0.04859261 | 0.18360508 |
| ACVR2A | | | | | | | | | |
| BMP2)- | 0.139868206 | 0.07920073 | −0.0201268 | −0.0778482 | 0.05275559 | 0.11376409 | 0.28224516 | −0.0170174 | 0.14262008 |
| ACVR2A | | | | | | | | | |
| BMP6)- | −0.073198007 | 0.19622997 | 0.17939288 | 0.13948999 | −0.0281943 | 0.05621897 | 0.16938142 | 0.03511719 | 0.28847371 |
| ACVR2A | | | | | | | | | |
| GDF11)- | −0.086340175 | 0.07918814 | 0.08892587 | 0.21188351 | −0.0575457 | 0.21301764 | 0.26642659 | −0.0132449 | 0.24456318 |
| ACVR2A | | | | | | | | | |
| FLT3LG)- | 0.003241356 | −0.1465203 | −0.0350969 | 0.10870012 | −0.0688338 | 0.1064354 | 0.11568683 | 0.18970305 | 0.05841954 |
| FLT3 | | | | | | | | | |
| CD55)- | 0.101924747 | −0.1877759 | 0.18194016 | −0.1787304 | 0.22989226 | 0.16032504 | −0.2532669 | −0.046369 | 0.34724226 |
| ADGRE5 | | | | | | | | | |
| CALM2)- | −0.053089374 | −0.0014647 | −0.0909795 | −0.0850884 | −0.1003939 | −0.0551348 | 0.13041519 | −0.0218602 | −0.0420153 |
| PDE1B | | | | | | | | | |
| CALM1)- | −0.034120097 | −0.1753778 | −0.10545 | 0.11663327 | −0.1399488 | 0.07652681 | −0.1429395 | 0.04538316 | −0.0424196 |
| PDE1B | | | | | | | | | |
| CALM3)- | −0.012570895 | −0.0082377 | 0.01255427 | −0.0051793 | 0.05292813 | 0.05986635 | −0.1299039 | −0.1248394 | −0.3364248 |
| PDE1B | | | | | | | | | |
| APOA2)- | −0.056246751 | −0.2356584 | −0.2020332 | −0.2218559 | −0.1277196 | −0.3131941 | −0.1522695 | −0.3872071 | −0.1658816 |
| LRP1 | | | | | | | | | |
| AGRN)- | −0.06427227 | 0.11420503 | −0.0451156 | 0.17614956 | −0.0186954 | −0.1683873 | 0.00302601 | 0.25370797 | −0.0200688 |
| LRP1 | | | | | | | | | |
| GPC3)- | −0.034615525 | −0.0889272 | −0.0551869 | −0.1734421 | −0.0839115 | −0.1318142 | −0.0641085 | −0.3857195 | −0.1315326 |
| LRP1 | | | | | | | | | |
| PDGFB)- | 0.145499025 | −0.1527091 | −0.0657493 | −0.0915341 | −0.0722331 | −0.1266296 | −0.2322247 | −0.2118662 | −0.2949149 |
| LRP1 | | | | | | | | | |
| A2M)- | 0.029990596 | 0.25774739 | 0.16865179 | −0.0863374 | 0.04105235 | 0.16790194 | 0.07393629 | −0.335798 | 0.13959974 |
| LRP1 | | | | | | | | | |
| APOC2)- | −0.055581119 | −0.1219261 | −0.1503685 | −0.2094731 | −0.0482606 | −0.1975253 | −0.1715826 | −0.2284187 | −0.1578553 |
| LRP1 | | | | | | | | | |
| SERPINA1)- | −0.076502924 | 0.09979334 | 0.02420802 | 0.16301183 | 0.00686932 | −0.1204015 | −0.0186628 | 0.0228088 | −0.0912749 |
| LRP1 | | | | | | | | | |
| SERPINE2)- | −0.196684976 | 0.06847724 | 0.02413352 | −0.0778181 | −0.1307122 | 0.06632026 | −0.0375541 | −0.3867834 | −0.0188415 |
| LRP1 | | | | | | | | | |
| HSPG2)- | −0.013053954 | 0.15747206 | 0.11683596 | 0.04325434 | 0.02597026 | 0.60143055 | 0.10182644 | −0.027244 | 0.20096316 |
| LRP1 | | | | | | | | | |
| MMP13)- | −0.017170413 | −0.1367969 | −0.1246688 | −0.0493944 | 0.00198229 | −0.1874186 | −0.0936185 | −0.1621338 | −0.1383401 |
| LRP1 | | | | | | | | | |
| APOE)- | 0.266543038 | 0.17967515 | 0.19179487 | 0.04127127 | −0.0344167 | −0.2667657 | 0.00587917 | −0.3760868 | 0.04316062 |
| LRP1 | | | | | | | | | |
| PCSK9)- | 0.128323861 | 0.0417451 | −0.0525885 | −0.0466847 | 0.01092615 | −0.2314433 | 0.1060863 | −0.0546391 | −0.0480822 |
| LRP1 | | | | | | | | | |
| MMP9)- | 0.12868686 | 0.04366841 | 0.15088773 | −0.1032798 | −0.1822547 | −0.2307718 | −0.2681069 | −0.1828129 | −0.2316039 |
| LRP1 | | | | | | | | | |
| HSP90B1)- | 0.031514402 | 0.19877429 | 0.29943812 | 0.19017028 | 0.01748825 | 0.35398864 | −0.0681803 | 0.2089498 | −0.0316807 |
| LRP1 | | | | | | | | | |
| MDK)- | −0.093344544 | −0.1473834 | −0.1214518 | −0.1953227 | −0.2521058 | 0.10531334 | 0.23589342 | 0.20719212 | 0.39980901 |
| LRP1 | | | | | | | | | |
| PLAU)- | −0.018158808 | 0.16781719 | 0.17874589 | −0.0616497 | −0.1086204 | 0.43294673 | 0.08884378 | −0.0911971 | 0.15771782 |
| LRP1 | | | | | | | | | |
| LRPAP1)- | 0.120338648 | −0.0608908 | −0.0724695 | −0.1174073 | −0.075282 | −0.246472 | −0.2492296 | −0.1686904 | −0.3134852 |
| LRP1 | | | | | | | | | |
| C3)- LRP1 | 0.131633066 | 0.12341889 | 0.10957076 | 0.05840791 | 0.11433501 | 0.00893927 | 0.02933138 | −0.1204916 | 0.11102714 |
| THBS1)- | −0.196611193 | 0.15797876 | 0.13304763 | −0.0462171 | −0.0458965 | 0.55543268 | −0.0183376 | −0.3340286 | 0.06039089 |
| LRP1 | | | | | | | | | |
| HSP90AA1)- | −0.11747715 | 0.18021771 | 0.05728598 | 0.13224714 | −0.0316192 | −0.0577543 | 0.21131924 | 0.36882231 | 0.37365599 |
| LRP1 | | | | | | | | | |
| APOA4)- | −0.051707593 | −0.2329751 | −0.1968269 | −0.2599089 | −0.149224 | −0.1988119 | −0.1125136 | −0.2591334 | −0.1750695 |
| LRP1 | | | | | | | | | |
| CALR)- | 0.198024921 | 0.15125253 | 0.19827547 | 0.15272714 | 0.06499352 | 0.37457944 | −0.106405 | 0.09422311 | −0.0935391 |
| LRP1 | | | | | | | | | |
| C1QB)- | 0.096883208 | 0.05122391 | 0.14590728 | 0.09024067 | 0.02786379 | −0.2023113 | −0.0095438 | −0.2776599 | 0.06114079 |
| LRP1 | | | | | | | | | |
| C4BPA)- | −0.0383104 | 0.05841157 | 0.03142408 | −0.1674221 | −0.0455672 | −0.241373 | −0.2472417 | −0.2641995 | −0.282015 |
| LRP1 | | | | | | | | | |
| VWF)- | 0.057213722 | −0.0392135 | −0.0221394 | −0.0730943 | 0.03983723 | −0.3047574 | −0.0662234 | −0.3280466 | −0.1126558 |
| LRP1 | | | | | | | | | |
| CCN2)- | −0.398453246 | 0.27920533 | 0.27829513 | −0.1437353 | −0.1741866 | 0.66438995 | 0.2674156 | −0.4221672 | 0.31350183 |
| LRP1 | | | | | | | | | |
| HPX)- | 0.079095571 | −0.3222815 | −0.3157383 | −0.0734674 | 0.07099753 | −0.2805141 | −0.2032721 | −0.2199145 | −0.263305 |
| LRP1 | | | | | | | | | |
| APP)- LRP1 | 0.001470603 | 0.07461058 | 0.14074403 | −0.1193497 | −0.0154493 | 0.09565296 | 0.0953409 | 0.3144686 | 0.19486431 |
| LPL)- LRP1 | 0.039594698 | −0.044725 | 0.0634449 | −0.2183082 | −0.1697978 | −0.0918429 | −0.1754439 | −0.2727759 | −0.2261062 |
| LIPC)- | 0.124421907 | −0.0466857 | −0.019829 | −0.2075994 | −0.1609408 | −0.2864108 | −0.1404557 | −0.259029 | −0.1654717 |
| LRP1 | | | | | | | | | |
| PF4)- LRP1 | 0.031948351 | −0.3760239 | −0.3155395 | −0.0496191 | 0.04986385 | −0.1930187 | −0.2031523 | −0.1430369 | −0.230371 |
| IL9)- IL9R | 0.059574739 | 0.17305254 | 0.22921985 | 0.08416204 | −0.0078356 | 0.21430194 | 0.10939737 | 0.15409518 | 0.45986885 |
| APP)- | −0.10357733 | 0.04108128 | −0.0907104 | 0.03859398 | −0.0623926 | 0.04727601 | −0.0507204 | −0.1987886 | −0.1935757 |
| PTGER2 | | | | | | | | | |
| F12)- CD93 | 0.095102603 | 0.30035402 | −0.0051933 | 0.16807513 | −0.1199544 | 0.07278933 | −0.1241176 | 0.2295238 | 0.24481946 |
| COL4A3)- | −0.003347703 | 0.16080606 | 0.04063607 | 0.14781947 | −0.0367292 | 0.08590942 | −0.0637225 | 0.12612322 | 0.35674311 |
| CD93 | | | | | | | | | |
| COL4A5)- | −0.033606257 | 0.17550157 | 0.04967341 | −0.0833709 | 0.14494529 | −0.0277407 | 0.03876817 | 0.00456685 | 0.2540194 |
| CD93 | | | | | | | | | |
| KNG1)- | −0.027904076 | 0.15765915 | 0.17504731 | 0.15000733 | −0.1360583 | 0.06765151 | −0.1624771 | 0.23929028 | 0.43166146 |
| CD93 | | | | | | | | | |
| SFTPA2)- | 0.25928014 | 0.33124461 | −0.2850276 | 0.04482778 | 0.10119822 | −0.0696335 | −0.0002491 | 0.04924254 | 0.37765673 |
| CD93 | | | | | | | | | |
| COL4A2)- | −0.036610926 | −0.0116909 | 0.13852744 | −0.2919099 | 0.16020966 | −0.0508568 | 0.0305087 | −0.0047983 | 0.14206037 |
| CD93 | | | | | | | | | |
| COL4A4)- | −0.001016978 | 0.05483443 | −0.1254691 | 0.22243544 | −0.0081988 | 0.2437441 | −0.0117804 | 0.1186887 | 0.26309854 |
| CD93 | | | | | | | | | |
| MBL2)- | 0.1578066 | 0.34338217 | −0.0886789 | 0.0856345 | 0.02823416 | 0.08404098 | −0.0234743 | 0.12410179 | 0.33273673 |
| CD93 | | | | | | | | | |
| COL1A2)- | 0.093574789 | 0.07872844 | −0.0787289 | −0.232609 | 0.11332864 | −0.1402481 | −0.0341952 | 0.01279535 | 0.07245383 |
| CD93 | | | | | | | | | |
| C1QA)- | −0.040134769 | −0.1700246 | 0.1996568 | 0.2967754 | −0.269705 | 0.3826009 | −0.277664 | 0.31214445 | 0.42563039 |
| CD93 | | | | | | | | | |
| CCL21)- | −0.11428142 | 0.12319103 | 0.2417844 | 0.20519265 | 0.02101051 | 0.27212434 | 0.02942353 | 0.10079475 | 0.33612158 |
| CCR7 | | | | | | | | | |
| CCL19)- | −0.035664107 | 0.07172536 | 0.12987459 | 0.12642109 | −0.0611561 | 0.13153218 | 0.18271273 | 0.33585261 | 0.33535262 |
| CCR7 | | | | | | | | | |
| HLA-B)- | 0.258341011 | −0.0494709 | 0.36837141 | 0.0690735 | 0.08481594 | 0.09746509 | −0.2165536 | −0.1919596 | 0.36239806 |
| CANX | | | | | | | | | |
| TNF)- | 0.058779397 | −0.0214619 | 0.08605656 | 0.11955473 | −0.0550465 | 0.26458971 | −0.0450267 | −0.0092434 | 0.03343547 |
| TRAF2 | | | | | | | | | |
| FADD)- | −0.008423183 | −0.0616772 | 0.0495544 | 0.04535186 | 0.00034442 | 0.03034388 | −0.039609 | 0.07809117 | 0.07499249 |
| TRAF2 | | | | | | | | | |
| GSTP1)- | −0.065644085 | 0.05891486 | −0.0067049 | −0.0009725 | 0.00161176 | −0.0359979 | 0.03403455 | 0.01594961 | 0.04540774 |
| TRAF2 | | | | | | | | | |
| F2)- F2RL3 | 0.027586491 | 0.17792527 | 0.15421057 | 0.12781816 | 0.05001401 | 0.26424833 | 0.15921247 | 0.09709804 | 0.49593628 |
| GAL)- | 0.120038867 | 0.07273639 | 0.2040021 | 0.11375874 | 0.12092407 | 0.20818968 | 0.15321157 | 0.1634922 | 0.17276117 |
| GALR3 | | | | | | | | | |
| S100A9)- | −0.388220753 | −0.4173657 | 0.48520713 | 0.43607992 | −0.3233072 | 0.44088401 | −0.3894862 | 0.39373008 | 0.44274934 |
| CD68 | | | | | | | | | |
| S100A8)- | −0.376574254 | −0.4212672 | 0.41000842 | 0.42343539 | −0.2618288 | 0.42568818 | −0.3595705 | 0.45162519 | 0.45530442 |
| CD68 | | | | | | | | | |
| PDGFB)- | −0.047578782 | 0.17084042 | 0.10678021 | 0.20447434 | 0.16972581 | 0.16698237 | 0.37332335 | 0.15538193 | 0.38895277 |
| ART1 | | | | | | | | | |
| AFDN)- | 0.195297326 | −0.1262785 | 0.22729683 | −0.1032425 | 0.13794331 | 0.14311933 | −0.2983017 | −0.248666 | 0.2040952 |
| NECTIN2 | | | | | | | | | |
| ADCYAP1)- | 0.03021386 | 0.21380266 | 0.04136209 | 0.16212439 | −0.0818256 | 0.18551813 | −0.0332886 | 0.11416006 | 0.44419056 |
| RAMP2 | | | | | | | | | |
| GHRH)- | −0.007489001 | 0.27703517 | 0.09901478 | 0.38209174 | −0.0542612 | 0.20153605 | 0.11077625 | 0.09928037 | 0.38236255 |
| RAMP2 | | | | | | | | | |
| CALCA)- | 0.061977161 | 0.0763734 | 0.11952424 | 0.28036864 | −0.1434809 | 0.18840575 | 0.02572522 | 0.23835693 | 0.4470845 |
| RAMP2 | | | | | | | | | |
| ADM)- | −0.046978636 | 0.0881894 | 0.06962842 | 0.20067784 | 0.12577554 | −0.033061 | −0.0379592 | −0.0918465 | 0.07257324 |
| RAMP2 | | | | | | | | | |
| GCG)- | 0.137934783 | −0.0274307 | −0.0250995 | 0.14491437 | −0.1740326 | 0.02823792 | −0.0723876 | 0.09886576 | 0.33871419 |
| RAMP2 | | | | | | | | | |
| TSHB)- | −0.055595362 | 0.12709339 | 0.22115868 | 0.27025352 | −0.0611664 | 0.27325195 | 0.06509993 | 0.08159286 | 0.49624375 |
| RAMP2 | | | | | | | | | |
| PTH)- | 0.041933905 | 0.16916236 | 0.10023071 | 0.26253173 | 0.03418003 | 0.17611464 | 0.05548841 | 0.00806654 | 0.46672037 |
| RAMP2 | | | | | | | | | |
| ADM2)- | −0.055184756 | 0.13063088 | 0.04986984 | −0.1023773 | −0.0878163 | 0.27301034 | 0.02266307 | 0.13053715 | −0.0768548 |
| RAMP2 | | | | | | | | | |
| PTHLH)- | −0.124962085 | 0.20850401 | 0.13188997 | 0.00154435 | −0.0282978 | 0.07886202 | 0.00780931 | 0.154759 | 0.40234178 |
| RAMP2 | | | | | | | | | |
| NPS)- | 0.114200414 | 0.18176837 | −0.0498834 | 0.11995295 | −0.0974134 | 0.16896999 | 0.08369267 | −0.009599 | 0.55980109 |
| RAMP2 | | | | | | | | | |
| FSHB)- | 0.054043439 | 0.07226215 | 0.0309262 | 0.25006301 | −0.1011207 | 0.21464737 | 0.08045614 | 0.05721293 | 0.41909393 |
| RAMP2 | | | | | | | | | |
| POMC)- | −0.037214527 | 0.24582415 | 0.03061681 | 0.21198136 | −0.1073334 | 0.29220752 | −0.0134062 | 0.05176736 | 0.43394021 |
| RAMP2 | | | | | | | | | |
| CALCB)- | 0.053449681 | 0.10851292 | −0.042914 | 0.31906064 | −0.1326292 | 0.24129139 | 0.07732814 | 0.04030344 | 0.58267629 |
| RAMP2 | | | | | | | | | |
| GIP)- | 0.050876764 | 0.24289565 | 0.14732247 | 0.18585122 | −0.152986 | 0.23655715 | −0.0843578 | 0.05013969 | 0.44344031 |
| RAMP2 | | | | | | | | | |
| CRH)- | −0.119924892 | 0.10734816 | −0.099271 | 0.20720821 | −0.1078497 | 0.33605104 | −0.0254366 | 0.23744567 | 0.41614401 |
| RAMP2 | | | | | | | | | |
| INSL3)- | 0.02158513 | 0.33462669 | −0.0320774 | 0.0140313 | −0.0328172 | 0.0616898 | −0.1165083 | 0.05917928 | 0.48547955 |
| RAMP2 | | | | | | | | | |
| CALCA)- | 0.015130773 | 0.07816489 | −0.0221845 | −0.0616778 | −0.0083196 | −0.0064943 | 0.03526404 | −0.1001534 | 0.02626742 |
| RAMP1 | | | | | | | | | |
| ADM)- | 0.020218557 | 0.06494489 | 0.01921989 | −0.0387376 | −0.0414564 | −0.1056146 | −0.0638883 | −0.1208743 | 0.00181033 |
| RAMP1 | | | | | | | | | |
| ADM2)- | −0.170904742 | −0.0536424 | 0.00098784 | 0.01880057 | −0.0825561 | 0.03465492 | −0.2276915 | 0.13581992 | −0.1871765 |
| RAMP1 | | | | | | | | | |
| CALCB)- | 0.02995849 | −0.0330463 | 0.09757796 | −0.2138464 | −0.1195613 | 0.05373798 | −0.0434611 | −0.1977298 | −0.0575412 |
| RAMP1 | | | | | | | | | |
| SST)- | −0.014620231 | 0.05500783 | 0.01546432 | 0.20522658 | 0.08410633 | 0.22332972 | −0.0353862 | 0.1636422 | 0.30403178 |
| SSTR4 | | | | | | | | | |
| NMS)- | 0.078453298 | 0.02654332 | 0.13166948 | 0.03117395 | 0.00843415 | 0.15404782 | 0.14294539 | 0.10489907 | 0.10714594 |
| NMUR2 | | | | | | | | | |
| NMU)- | −0.02047347 | −0.1197811 | 0.18813748 | −0.0862321 | −0.0181602 | 0.20043819 | 0.08274289 | 0.02141521 | 0.38087678 |
| NMUR2 | | | | | | | | | |
| ARF1)- | −0.114761374 | 0.02414057 | −0.0146417 | 0.00339803 | 0.01972741 | −0.1833741 | −0.1917809 | −0.089659 | −0.478509 |
| CHRM3 | | | | | | | | | |
| VEGFC)- | 0.011435704 | 0.17906923 | 0.13617362 | −0.1107965 | 0.04015282 | 0.09746456 | 0.18164038 | 0.13785143 | 0.53040067 |
| LYVE1 | | | | | | | | | |
| PCSK9)- | −0.03873203 | 0.03495383 | −0.028172 | 0.06345862 | 0.08768805 | −0.1845371 | 0.1369266 | −0.0512558 | 0.10409273 |
| SORT1 | | | | | | | | | |
| LRPAP1)- | −0.065334037 | −0.0099144 | 0.02794618 | −0.0041837 | 0.02132786 | −0.061469 | −0.0350298 | −0.104788 | −0.0179861 |
| SORT1 | | | | | | | | | |
| BDNF)- | 0.079223497 | −0.1773046 | 0.01346044 | −0.0116774 | −0.0740634 | −0.065485 | −0.0627357 | −0.2058952 | −0.2162838 |
| SORT1 | | | | | | | | | |
| DLK1)- | 0.045533059 | 0.07589098 | −0.0120667 | 0.04936355 | 0.08718084 | 0.0117352 | 0.12413452 | 0.04229627 | −0.1060885 |
| NOTCH2 | | | | | | | | | |
| DLL1)- | −0.077652451 | 0.07822631 | 0.12492326 | −0.0162897 | −0.0738426 | 0.20483066 | 0.02612822 | −0.0247983 | 0.12697181 |
| NOTCH2 | | | | | | | | | |
| MFNG)- | 0.06349086 | 0.10660177 | 0.07105101 | −0.1016385 | 0.09699057 | 0.0135926 | −0.0229968 | 0.10187168 | 0.10307216 |
| NOTCH2 | | | | | | | | | |
| PSEN1)- | 0.036350772 | 0.04812091 | 0.07964322 | 0.14855045 | 0.10973779 | −0.1737514 | −0.0612738 | 0.0379389 | 0.00337754 |
| NOTCH2 | | | | | | | | | |
| ADAM10)- | −0.06227066 | 0.02016675 | 0.14815547 | −0.0539733 | 0.07763529 | −0.1548143 | 0.04972822 | 0.07112851 | 0.17024655 |
| NOTCH2 | | | | | | | | | |
| APP)- | −0.004939197 | −0.0630836 | 0.05216895 | −0.1950406 | −0.1162851 | 0.05854342 | 0.17390349 | −0.0231724 | 0.13273927 |
| NOTCH2 | | | | | | | | | |
| JAG2)- | 0.228382982 | −0.1508657 | −0.0996781 | −0.0155233 | 0.03500045 | 0.04573193 | 0.03022655 | −0.0545006 | 0.15898329 |
| NOTCH2 | | | | | | | | | |
| DSC2)- | 0.013754203 | 0.06865399 | 0.07346281 | −0.1072806 | −0.0469335 | 0.12820182 | 0.04135712 | 0.02872802 | 0.12409868 |
| DSG1 | | | | | | | | | |
| PDGFC)- | −0.121215051 | 0.23888292 | 0.25233638 | −0.127063 | −0.2435934 | 0.40019776 | 0.10131518 | −0.1295392 | 0.36319484 |
| PDGFRA | | | | | | | | | |
| PDGFB)- | 0.001931841 | 0.02537921 | 0.01105297 | 0.02663725 | −0.1140705 | 0.08351438 | 0.02998176 | −0.0220153 | 0.32589572 |
| PDGFRA | | | | | | | | | |
| PDGFD)- | −0.064027517 | 0.16068342 | 0.07145651 | −0.0740598 | 0.09403733 | 0.17583519 | 0.00178404 | −0.0943765 | 0.3169057 |
| PDGFRA | | | | | | | | | |
| PDGFA)- | −0.032174229 | 0.27977136 | −0.0048227 | −0.0367091 | −0.0674457 | 0.13998564 | −0.1444194 | −0.0030379 | 0.0623009 |
| PDGFRA | | | | | | | | | |
| FGF19)- | −0.001434853 | 0.30455633 | 0.23742713 | 0.09756806 | −0.0440738 | 0.0504148 | 0.05044177 | 0.06078429 | 0.36421597 |
| KLB | | | | | | | | | |
| FGF21)- | 0.005473947 | 0.08331844 | 0.08243793 | −0.1300292 | −0.0340688 | 0.05347947 | 0.00204545 | 0.02709532 | 0.40517557 |
| KLB | | | | | | | | | |
| LAMA1)- | −0.061225746 | 0.07932878 | 0.29200082 | 0.12914583 | −0.0252283 | 0.06428693 | 0.09378296 | 0.0929746 | 0.02306565 |
| NT5E | | | | | | | | | |
| FN1)- NT5E | −0.061552035 | −0.1254661 | 0.05286218 | 0.06266771 | −0.032392 | 0.01145778 | 0.04479355 | 0.01755867 | 0.02960674 |
| CALM1)- | 0.067924579 | −0.1069385 | −0.1656667 | −0.0267394 | −0.0687874 | −0.0196139 | −0.12738 | 0.01067873 | −0.0856305 |
| MIP | | | | | | | | | |
| SEMA7A)- | −0.115666164 | 0.07866205 | 0.09031634 | 0.0678848 | 0.07046365 | −0.0326167 | 0.08274296 | 0.19953674 | 0.30152211 |
| PLXNC1 | | | | | | | | | |
| LTA)- | 0.05899486 | 0.14864547 | 0.00972411 | −0.2020055 | −0.0034322 | 0.01879172 | −0.0863531 | 0.09142429 | −0.1075974 |
| RIPK1 | | | | | | | | | |
| TNF)- | −0.052591094 | 0.12736864 | 0.10417549 | −0.0871124 | 0.07071896 | 0.14399357 | 0.04110023 | 0.13375557 | −0.0009608 |
| RIPK1 | | | | | | | | | |
| CD14)- | 0.088641646 | −0.0432926 | 0.11569126 | 0.01973208 | −0.1126959 | 0.06457557 | −0.0993527 | −0.0173046 | −0.1070172 |
| RIPK1 | | | | | | | | | |
| CALR)- | −0.063177876 | −0.1533705 | 0.21244846 | 0.02783754 | 0.05284858 | 0.10149294 | −0.0922097 | 0.13998055 | 0.36513405 |
| HLA-F | | | | | | | | | |
| B2M)- | 0.109562477 | −0.1319249 | 0.64407658 | 0.53398677 | 0.00637425 | 0.65372295 | −0.2903795 | 0.49585208 | 0.82608033 |
| HLA-F | | | | | | | | | |
| HMGB1)- | 0.02415321 | 0.0623864 | 0.03133107 | 0.10931737 | −0.0309267 | 0.05249837 | 0.14014942 | −0.0771813 | −0.2389982 |
| TLR2 | | | | | | | | | |
| CCN1)- | 0.267379985 | 0.19599118 | −0.273599 | −0.2008152 | 0.17210339 | −0.214881 | −0.2988203 | 0.28013669 | 0.30871577 |
| TLR2 | | | | | | | | | |
| HSP90B1)- | 0.032321004 | −0.1031166 | −0.0825429 | −0.0428213 | 0.02642809 | −0.0287964 | 0.30104153 | −0.1494705 | 0.03826877 |
| TLR2 | | | | | | | | | |
| BGN)- | 0.224094444 | 0.18396485 | −0.2837018 | −0.348257 | 0.23289187 | −0.244099 | −0.3273386 | 0.24834964 | 0.30207499 |
| TLR2 | | | | | | | | | |
| VCAN)- | 0.172978816 | 0.1417735 | −0.1989128 | −0.3361158 | 0.18663678 | −0.1780753 | −0.3512337 | 0.29053903 | 0.35864027 |
| TLR2 | | | | | | | | | |
| ZG16B)- | 0.105110657 | 0.23662901 | −0.0351567 | 0.215749 | −0.0039788 | 0.11924299 | −0.1051012 | −0.0314796 | −0.1911338 |
| TLR2 | | | | | | | | | |
| HRAS)- | −0.09409028 | 0.10441306 | 0.07322883 | 0.14269264 | −0.1347743 | 0.15590981 | −0.1768176 | 0.11972282 | 0.29452784 |
| TLR2 | | | | | | | | | |
| RNASE2)- | −0.099127737 | −0.0512536 | 0.238616 | 0.22445392 | −0.1242316 | 0.17158267 | −0.1849876 | 0.08368743 | 0.28211749 |
| TLR2 | | | | | | | | | |
| CCN6)- | −0.04122269 | −0.0972751 | 0.09258738 | −0.0436159 | 0.04637579 | 0.05792932 | −0.0588314 | 0.1399536 | −0.0282621 |
| SORL1 | | | | | | | | | |
| MDK)- | −0.029628979 | −0.2696569 | 0.04356774 | −0.2878259 | 0.0522575 | −0.1029959 | 0.12254674 | 0.13064093 | 0.29447675 |
| SORL1 | | | | | | | | | |
| LRPAP1)- | 0.050991152 | −0.107809 | 0.08465115 | −0.1087237 | 0.04614539 | 0.13794867 | 0.05425973 | 0.18633973 | −0.0533402 |
| SORL1 | | | | | | | | | |
| APP)- | −0.052854454 | 0.10122096 | −0.1665671 | 0.09689945 | −0.0148185 | −0.1920933 | 0.14808478 | −0.1329638 | −0.0665221 |
| SORL1 | | | | | | | | | |
| L1CAM)- | 0.163986063 | −0.0306236 | −0.1476385 | 0.06891096 | −0.0308349 | −0.1077376 | −0.0077541 | −0.0853252 | 0.10761155 |
| ITGAV | | | | | | | | | |
| TNC)- | −0.026299867 | 0.02943301 | 0.12714943 | −0.0631443 | −0.0628731 | 0.22283484 | 0.11296281 | −0.2328136 | 0.17743511 |
| ITGAV | | | | | | | | | |
| PLAU)- | −0.006916806 | 0.09188099 | 0.32629871 | −0.0513089 | 0.00975224 | 0.3958871 | 0.33630177 | 0.01526574 | 0.25960562 |
| ITGAV | | | | | | | | | |
| EDIL3)- | −0.179252289 | 0.03650568 | 0.15385145 | −0.0396461 | 0.01874972 | 0.31510128 | 0.23775996 | −0.3401699 | 0.20102761 |
| ITGAV | | | | | | | | | |
| COL4A5)- | −0.162915399 | 0.12278664 | 0.16620666 | −0.0029739 | −0.0425184 | 0.11470393 | 0.11583653 | −0.0251177 | 0.12537221 |
| ITGAV | | | | | | | | | |
| SPP1)- | 0.200740037 | 0.12635726 | 0.15483329 | −0.0702952 | −0.0499344 | −0.1538195 | −0.0004453 | −0.2665979 | 0.00551298 |
| ITGAV | | | | | | | | | |
| NID1)- | −0.11709405 | −0.0062719 | 0.18040324 | 0.10836796 | 0.01994147 | 0.14806677 | 0.14583632 | −0.236307 | −0.0332508 |
| ITGAV | | | | | | | | | |
| PDGFB)- | 0.135218312 | 0.08309067 | 0.12912921 | −0.0004307 | 0.04293478 | −0.0809661 | −0.1036262 | −0.0165549 | 0.04327524 |
| ITGAV | | | | | | | | | |
| COL4A4)- | 0.095795264 | 0.06954096 | 0.08134027 | −0.1815641 | 0.00823243 | −0.0465111 | −0.0732656 | −0.1597564 | 0.00778032 |
| ITGAV | | | | | | | | | |
| CALR)- | 0.211767222 | −0.1735187 | 0.02467461 | −0.1336454 | −0.014458 | 0.29425327 | −0.0911476 | 0.24309146 | −0.1121461 |
| ITGAV | | | | | | | | | |
| CCN1)- | −0.346502125 | 0.17896553 | 0.30150879 | −0.2075246 | −0.1545194 | 0.4475309 | 0.22221376 | −0.2959107 | 0.29044221 |
| ITGAV | | | | | | | | | |
| FGG)- | −0.088630912 | −0.0294464 | −0.0598302 | 0.06492689 | 0.08946515 | −0.0558728 | 0.01903219 | −0.194491 | 0.04289119 |
| ITGAV | | | | | | | | | |
| FGA)- | 0.047510873 | 0.02466031 | −0.1324861 | 0.11146692 | −0.0172876 | −0.0562397 | −0.0278056 | −0.0755904 | −0.0684465 |
| ITGAV | | | | | | | | | |
| COL4A3)- | −0.02937629 | −0.0501801 | −0.0099993 | −0.0208312 | −0.135171 | −0.0374064 | 0.01442725 | −0.0737041 | 0.05970569 |
| ITGAV | | | | | | | | | |
| MFGE8)- | −0.163659934 | 0.08768836 | 0.22417584 | −0.10229 | −0.0455944 | 0.47217942 | 0.11786056 | −0.271124 | 0.04931805 |
| ITGAV | | | | | | | | | |
| FGB)- | 0.091095605 | 0.17149978 | 0.09757447 | −0.0771698 | −0.1093762 | −0.224259 | −0.1695909 | −0.1025144 | −0.2029703 |
| ITGAV | | | | | | | | | |
| AZGP1)- | 0.061256212 | −0.0062844 | −0.1908764 | 0.04907544 | 0.10742195 | −0.0878317 | 0.04114992 | −0.2066311 | −0.0217947 |
| ITGAV | | | | | | | | | |
| ADAM9)- | 0.018987759 | −0.268535 | −0.0766402 | −0.289173 | −0.1568283 | 0.07735158 | 0.33708003 | 0.19869753 | 0.37695369 |
| ITGAV | | | | | | | | | |
| VEGFA)- | 0.015180966 | 0.10701245 | 0.20435897 | 0.10502881 | 0.2007593 | 0.03060349 | −0.0283271 | 0.10751056 | −0.1735713 |
| ITGAV | | | | | | | | | |
| ADAM15)- | −0.161997712 | −0.1368735 | 0.07882133 | −0.1401682 | −0.0935732 | 0.09147436 | −0.0665086 | −0.1594742 | −0.1641943 |
| ITGAV | | | | | | | | | |
| COL1A2)- | −0.356270489 | 0.15650192 | 0.32122671 | −0.1543374 | −0.0831593 | 0.76199384 | 0.31352537 | −0.296895 | 0.18531473 |
| ITGAV | | | | | | | | | |
| FN1)- | −0.266407503 | 0.19273177 | 0.38699708 | −0.119617 | −0.0760576 | 0.69843269 | 0.35113565 | −0.2438356 | 0.24292276 |
| ITGAV | | | | | | | | | |
| CP)- | 0.027320843 | −0.0392799 | −0.0013447 | −0.1103849 | 0.21646389 | 0.19532439 | 0.0758408 | 0.12286861 | −0.0474289 |
| SLC40A1 | | | | | | | | | |
| SOSTDC1)- | −0.064630473 | 0.10804709 | 0.28408585 | 0.05532499 | 0.02272774 | 0.0630068 | −0.0172795 | 0.00937868 | 0.14343778 |
| LRP6 | | | | | | | | | |
| IGFBP4)- | −0.021329833 | −0.0518756 | −0.071781 | 0.04107322 | 0.06023207 | −0.0099957 | −0.1609906 | 0.04066433 | −0.2088905 |
| LRP6 | | | | | | | | | |
| PTH)- LRP6 | −0.05462044 | 0.01524769 | 0.16826672 | 0.13193618 | −0.0619939 | 0.06690016 | 0.13430112 | −0.0002373 | 0.32540509 |
| RSPO3)- | 0.196531498 | 0.01878518 | 0.15755176 | −0.0449424 | −0.0669219 | −0.0395308 | 0.02677487 | −0.0101786 | 0.31136707 |
| LRP6 | | | | | | | | | |
| WNT2)- | 0.041667216 | 0.03943937 | 0.26033878 | 0.20322621 | −0.0209676 | −0.013039 | 0.07933097 | −0.0162933 | 0.26160018 |
| LRP6 | | | | | | | | | |
| CCN2)- | −0.101884965 | 0.11879897 | 0.13411635 | −0.0097607 | −0.1411001 | 0.04873997 | 0.09432207 | −0.0791898 | 0.14607982 |
| LRP6 | | | | | | | | | |
| SOST)- | 0.167328154 | 0.04505871 | 0.11191992 | 0.18240361 | 0.1606195 | −0.0773139 | 0.14670847 | −0.0448146 | 0.29114946 |
| LRP6 | | | | | | | | | |
| APP)- LRP6 | −0.048538982 | −0.1811018 | 0.17047436 | −0.2043536 | 0.20332846 | 0.14924166 | −0.1392492 | −0.0614755 | −0.0106774 |
| CKLF)- | 0.009771486 | −0.0018414 | 0.09297862 | 0.00154271 | 0.01630536 | −0.0932539 | 0.11773926 | 0.10836301 | −0.0521245 |
| LRP6 | | | | | | | | | |
| WNT5A)- | 0.064553238 | 0.06456079 | 0.12716611 | −0.0357368 | −0.0152238 | 0.06246153 | 0.23109015 | −0.0998283 | 0.1479138 |
| LRP6 | | | | | | | | | |
| DKK1)- | 0.105652355 | 0.10309691 | −0.0245077 | 0.08357723 | −0.0660222 | 0.09185891 | −0.0800063 | 0.10324182 | 0.11523725 |
| LRP6 | | | | | | | | | |
| WNT7A)- | 0.100554231 | 0.0650804 | 0.07477478 | 0.00033835 | −0.0926927 | 0.20995804 | −0.0971642 | −0.1142803 | −0.1695032 |
| LRP6 | | | | | | | | | |
| DKK2)- | 0.062266399 | −0.0228256 | 0.06255357 | 0.02135218 | 0.08109347 | 0.0648474 | 0.10704644 | −0.0630405 | 0.15604339 |
| LRP6 | | | | | | | | | |
| DSC2)- | 0.220828146 | −0.2652125 | 0.30756127 | −0.1667565 | 0.30660818 | 0.29740624 | −0.2921869 | −0.2541695 | 0.76838272 |
| DSG2 | | | | | | | | | |
| PDGFC)- | −0.138906914 | 0.24143745 | 0.21501051 | −0.2812161 | −0.2772165 | 0.40256677 | 0.19380808 | −0.1330894 | 0.35588325 |
| PDGFRB | | | | | | | | | |
| PDGFB)- | 0.160477156 | −0.0728165 | −0.0944396 | −0.0413124 | −0.0792766 | 0.00156311 | −0.1698165 | 0.02905195 | 0.0659894 |
| PDGFRB | | | | | | | | | |
| PDGFD)- | 0.003657826 | 0.0683508 | 0.02096896 | −0.1684151 | −0.0651874 | 0.16457581 | 0.02600279 | −0.0447156 | 0.28469488 |
| PDGFRB | | | | | | | | | |
| MFGE8)- | −0.230721344 | 0.40687176 | 0.2879768 | −0.3678011 | −0.2058273 | 0.38796164 | 0.19667905 | −0.2904496 | 0.43648645 |
| PDGFRB | | | | | | | | | |
| PDGFA)- | 0.022834817 | 0.16702606 | −0.0023089 | −0.075812 | −0.0679682 | 0.03674484 | −0.1920008 | 0.0296435 | −0.1125652 |
| PDGFRB | | | | | | | | | |
| PDGFC)- | 0.249108616 | 0.11850713 | 0.07747598 | 0.06101945 | 0.0301951 | 0.02935015 | −0.0066126 | 0.23651835 | 0.34995774 |
| FLT4 | | | | | | | | | |
| VEGFC)- | 0.163253377 | 0.15193052 | 0.04733078 | 0.04782388 | −0.0306327 | 0.11737091 | 0.05940994 | 0.21748569 | 0.36935736 |
| FLT4 | | | | | | | | | |
| TNF)- FLT4 | 0.072772886 | 0.25052015 | 0.16006715 | 0.09736668 | 0.00594827 | 0.16801012 | 0.06218693 | 0.16829089 | 0.44793783 |
| COL1A2)- | 0.154844021 | −0.0523705 | −0.0544956 | −0.1292948 | 0.03391913 | −0.2027169 | −0.0986502 | 0.15881949 | −0.0047426 |
| FLT4 | | | | | | | | | |
| FN1)- FLT4 | 0.060856586 | −0.1404143 | −0.0658069 | −0.0185662 | −0.0008324 | −0.1323063 | −0.1150499 | 0.07256327 | −0.0384064 |
| LGALS3BP)- | 0.09360746 | 0.1941677 | −0.0012909 | 0.2395606 | 0.02855259 | 0.15432209 | 0.01558626 | −0.0137835 | −0.0779026 |
| CD33 | | | | | | | | | |
| IL12A)- | 0.070507354 | 0.30965667 | 0.23588524 | 0.06808314 | 0.07129354 | 0.14200627 | 0.34514623 | 0.10753071 | 0.42672721 |
| IL12RB2 | | | | | | | | | |
| F2)- | 0.046398642 | 0.2696547 | 0.25006158 | 0.07335767 | 0.13100895 | 0.15929057 | 0.1579105 | 0.21281996 | 0.46549621 |
| ITGA2B | | | | | | | | | |
| CALR)- | −0.149735252 | −0.1891557 | −0.0639198 | −0.1219105 | −0.0987218 | −0.1343099 | −0.1018881 | −0.0984014 | −0.2359334 |
| ITGA2B | | | | | | | | | |
| FGG)- | −0.005060772 | 0.07322777 | 0.16619534 | 0.295473 | −0.0473396 | 0.25102837 | 0.22343542 | 0.1667707 | 0.44574669 |
| ITGA2B | | | | | | | | | |
| FGA)- | 0.046832532 | −0.0415881 | 0.05291129 | 0.25894393 | 0.11289938 | 0.22816561 | 0.08112122 | 0.16245417 | 0.37092824 |
| ITGA2B | | | | | | | | | |
| FGB)- | −0.035792023 | −0.0837501 | 0.0836661 | 0.12739343 | 0.0029012 | 0.17449723 | 0.14702605 | −0.0166352 | 0.31966606 |
| ITGA2B | | | | | | | | | |
| COL1A2)- | −0.020901616 | −0.2204783 | −0.0913009 | −0.2015376 | 0.03187873 | −0.1565719 | −0.1237453 | −0.0523053 | −0.1855022 |
| ITGA2B | | | | | | | | | |
| FN1)- | 0.02616145 | −0.1024677 | −0.0816283 | −0.181807 | −0.0274105 | −0.0716413 | −0.0879595 | 0.02326927 | −0.0781777 |
| ITGA2B | | | | | | | | | |
| GCG)- | 0.003675615 | −0.0940047 | −0.0090285 | 0.08741188 | −0.0935676 | 0.34428322 | 0.0377243 | 0.36787415 | 0.37613676 |
| GLP2R | | | | | | | | | |
| CALM1)- | −0.021285823 | −0.2585673 | 0.00606529 | 0.12127235 | −0.0054029 | −0.1006845 | 0.01164746 | −0.0217145 | −0.0006239 |
| GLP2R | | | | | | | | | |
| CALM1)- | 0.023208463 | 0.05898199 | 0.0288572 | 0.15211344 | −0.0731459 | −0.0891182 | 0.09526655 | 0.08007944 | −0.2050508 |
| KCNN4 | | | | | | | | | |
| CALR)- | −0.100898307 | −0.0240562 | 0.05560161 | −0.197196 | 0.02082928 | −0.0836461 | 0.04589564 | 0.04070135 | −0.0205025 |
| SCARF1 | | | | | | | | | |
| ARF1)- | −0.056720074 | −0.090112 | 0.08283296 | −0.0743788 | 0.05547445 | −0.0338634 | 0.00934157 | 0.13046134 | 0.1370284 |
| PLD2 | | | | | | | | | |
| HRAS)- | −0.064378012 | −0.1099632 | 0.13569959 | −0.0012278 | 0.00445082 | 0.02884594 | −0.1581284 | 0.14578957 | −0.0503864 |
| GRIN2D | | | | | | | | | |
| HSPA1A)- | 0.097782886 | 0.11017956 | −0.0731162 | 0.02030489 | 0.01538392 | 0.14594969 | 0.06763482 | 0.20951582 | 0.09949806 |
| GRIN2D | | | | | | | | | |
| IL27)- | 0.035056193 | 0.17547691 | 0.247221 | 0.15005479 | −0.1462164 | 0.2769561 | 0.14841072 | 0.22561815 | 0.37837679 |
| IL27RA | | | | | | | | | |
| DLK1)- | −0.231538043 | 0.19887336 | 0.25863008 | −0.0848819 | −0.0959728 | 0.04771559 | 0.05723375 | −0.1897892 | 0.02623504 |
| NOTCH3 | | | | | | | | | |
| DLL1)- | −0.046608949 | 0.05763751 | 0.11758096 | −0.138345 | −0.0837916 | 0.08610781 | 0.17196075 | −0.1625489 | 0.05644233 |
| NOTCH3 | | | | | | | | | |
| PSEN1)- | −0.067501908 | 0.00595579 | −0.000498 | 0.03282991 | 0.06842443 | −0.1395393 | 0.00016551 | 0.07787019 | 0.03698008 |
| NOTCH3 | | | | | | | | | |
| THBS2)- | −0.153026371 | 0.3149372 | 0.42738358 | −0.3149779 | −0.2156582 | 0.17871455 | 0.49649649 | −0.2765071 | 0.42159147 |
| NOTCH3 | | | | | | | | | |
| SCGB3A1)- | −0.01390023 | −0.1178143 | −0.0889354 | 0.02792817 | −0.0360876 | −0.0035933 | 0.11662954 | −0.1237312 | 0.07457781 |
| NOTCH3 | | | | | | | | | |
| JAG2)- | 0.08301506 | −0.0874852 | 0.01142983 | −0.0633557 | 0.01951634 | 0.06981428 | 0.15930568 | −0.0274457 | 0.31837376 |
| NOTCH3 | | | | | | | | | |
| CLCF1)- | 0.112817689 | 0.23900035 | 0.08952689 | 0.06199938 | 0.03885884 | 0.07349053 | 0.13669046 | 0.00016698 | 0.31422629 |
| LIFR | | | | | | | | | |
| CNTF)- | 0.079109865 | 0.09230729 | 0.11779657 | 0.16576472 | 0.09632512 | 0.17369207 | 0.01435623 | −0.0432457 | 0.37641889 |
| LIFR | | | | | | | | | |
| APP)- | 0.226869475 | −0.015798 | 0.25172209 | 0.02454782 | 0.11900403 | 0.09117832 | −0.1288843 | −0.0558094 | 0.38237659 |
| APLP2 | | | | | | | | | |
| PCSK9)- | −0.133503354 | −0.0176859 | −0.1558468 | −0.0338641 | 0.04801515 | 0.01806905 | 0.15371704 | 0.00862104 | −0.1385345 |
| APLP2 | | | | | | | | | |
| BMP7)- | 0.189110004 | 0.04098454 | 0.08689394 | −0.0108468 | 0.07592668 | −0.0915944 | 0.03235374 | −0.0334711 | −0.0527134 |
| ACVR1 | | | | | | | | | |
| INHBB)- | 0.192887383 | 0.03737565 | 0.14604127 | 0.02589828 | 0.22670215 | 0.1782231 | −0.0600877 | 0.10305747 | −0.0934727 |
| ACVR1 | | | | | | | | | |
| BMP2)- | 0.1726529 | 0.09814262 | 0.20373807 | 0.01167551 | 0.02156596 | −0.0494375 | 0.03457513 | 0.05549148 | −0.0267683 |
| ACVR1 | | | | | | | | | |
| BMP6)- | −0.03553467 | 0.0214379 | 0.22375438 | −0.018064 | −0.0991967 | 0.20202887 | 0.00161475 | 0.17106916 | −0.004602 |
| ACVR1 | | | | | | | | | |
| TGFB2)- | −0.011202564 | 0.05713616 | 0.20081128 | −0.0697525 | −0.0751386 | 0.00713504 | 0.23127906 | −0.0020262 | 0.24230538 |
| ACVR1 | | | | | | | | | |
| GPC3)- | 0.087969016 | 0.06953849 | 0.01903718 | −0.0739539 | −0.1241432 | −0.0362593 | −0.1525447 | 0.03135266 | 0.08430892 |
| CD81 | | | | | | | | | |
| CD99)- | −0.005546995 | 0.00116593 | 0.20799552 | 0.00705989 | −0.0648609 | 0.1053626 | −0.1313199 | 0.00799414 | 0.11958076 |
| CD81 | | | | | | | | | |
| VCAN)- | −0.177153777 | −0.0545857 | 0.02566364 | −0.1247861 | −0.0547271 | −0.0299421 | 0.03896279 | −0.1468644 | 0.0679758 |
| SELP | | | | | | | | | |
| CD34)- | 0.09167375 | −0.0090807 | 0.04939303 | 0.21045623 | 0.09411215 | 0.12003037 | −0.0248374 | 0.20900801 | 0.39920497 |
| SELP | | | | | | | | | |
| SERPING1)- | −0.132503965 | −0.0478796 | 0.07165356 | −0.052627 | −0.0183303 | 0.01300463 | 0.12958688 | 0.01194522 | 0.1265337 |
| SELP | | | | | | | | | |
| CD24)- | −0.196889617 | 0.14442037 | 0.12184589 | 0.1288186 | 0.01504248 | −0.1104955 | −0.1576824 | −0.0734791 | −0.2943128 |
| SELP | | | | | | | | | |
| GAS6)- | 0.06974666 | −0.0225464 | 0.10153996 | −0.0567996 | −0.1338271 | −0.1349601 | 0.09615034 | 0.2701884 | 0.06257076 |
| TYRO3 | | | | | | | | | |
| PROS1)- | 0.003714857 | 0.13032266 | 0.00970213 | 0.2195751 | 0.09721109 | −0.0336502 | −0.0119917 | 0.11044212 | −0.1163813 |
| TYRO3 | | | | | | | | | |
| VEGFA)- | −0.02542733 | 0.0103407 | 0.10145443 | 0.03094141 | 0.2168326 | 0.12936722 | −0.0506665 | −0.1232091 | −0.2156157 |
| TYRO3 | | | | | | | | | |
| PDGFC)- | 0.172550609 | 0.13348 | −0.08558 | 0.04093847 | 0.08990612 | 0.07443994 | −0.1264312 | 0.22747031 | 0.27791725 |
| KDR | | | | | | | | | |
| VEGFC)- | −0.010486954 | 0.07514356 | 0.23082887 | 0.08126687 | −0.088028 | 0.16508669 | 0.00276196 | 0.17063938 | 0.45111732 |
| KDR | | | | | | | | | |
| GREM1)- | −0.069037205 | 0.09634315 | 0.06530381 | −0.2282012 | 0.12836737 | −0.1425622 | 0.06514089 | 0.02204587 | 0.28767777 |
| KDR | | | | | | | | | |
| TIMP3)- | −0.055932153 | −0.0233321 | −0.0489384 | −0.1712402 | 0.10844029 | −0.1468932 | 0.04262041 | −0.0348532 | 0.15520469 |
| KDR | | | | | | | | | |
| CXCL8)- | 0.005627781 | −0.0296288 | 0.06591396 | 0.10241395 | −0.1710734 | 0.09932227 | −0.1282539 | −0.0167688 | 0.12915416 |
| KDR | | | | | | | | | |
| COL18A1)- | −0.2945365 | −0.0127021 | 0.26004603 | 0.07325869 | −0.155417 | 0.27952937 | 0.09277499 | −0.0692962 | 0.3174632 |
| KDR | | | | | | | | | |
| VEGFA)- | 0.01240902 | 0.09660179 | −0.0330339 | 0.12561956 | 0.00177587 | −0.0402801 | 0.12903359 | −0.2221335 | −0.3154043 |
| KDR | | | | | | | | | |
| TNFSF12)- | −0.035120361 | −0.0469071 | 0.18236315 | −0.0131917 | −0.100047 | 0.02787926 | 0.16711206 | 0.07684488 | 0.37514683 |
| TNFRSF8 | | | | | | | | | |
| AFDN)- | 0.089991958 | 0.05166956 | 0.11188756 | 0.07985224 | 0.14862952 | 0.07798305 | 0.04781425 | −0.0174635 | −0.2443127 |
| NECTIN1 | | | | | | | | | |
| WNT5A)- | 0.103645743 | 0.0088171 | −0.0243049 | −0.1170274 | 0.02000926 | 0.1230721 | 0.12061637 | −0.0616711 | 0.26182312 |
| MCAM | | | | | | | | | |
| LAMA1)- | −0.163158856 | 0.07597762 | 0.06668406 | 0.03953094 | −0.0834253 | 0.0895609 | 0.05585697 | −0.0475786 | −0.0061926 |
| GPC1 | | | | | | | | | |
| TDGF1)- | −0.053027829 | 0.0936368 | 0.22983531 | 0.08048966 | −0.1745534 | −0.0858583 | 0.1867917 | −0.064048 | 0.13414359 |
| GPC1 | | | | | | | | | |
| BMP2)- | −0.166259588 | 0.15162847 | 0.23368227 | 0.01502246 | −0.1112877 | 0.12356614 | −0.0737194 | −0.0618141 | −0.0423465 |
| GPC1 | | | | | | | | | |
| NRG1)- | −0.028362469 | 0.08973341 | 0.19601915 | 0.11677432 | −0.1708908 | −0.038262 | 0.14374499 | −0.0280919 | 0.16449407 |
| GPC1 | | | | | | | | | |
| APP)- | 0.024704161 | −0.0875293 | 0.17553728 | −0.0978399 | 0.06037712 | 0.10655697 | 0.15720561 | 0.21084886 | −0.1030538 |
| GPC1 | | | | | | | | | |
| SERPINC1)- | −0.129246271 | −0.0192345 | −0.0463246 | 0.12288348 | 0.01513698 | −0.0952149 | 0.04201784 | 0.03634992 | 0.05169103 |
| GPC1 | | | | | | | | | |
| COL18A1)- | 0.046663522 | 0.13068608 | 0.20835939 | 0.02079503 | 0.18059723 | 0.04912113 | 0.16084112 | −0.0136189 | 0.16499451 |
| GPC1 | | | | | | | | | |
| SLIT2)- | −0.052901667 | −0.0461203 | 0.07523037 | 0.07579372 | −0.085121 | 0.02249585 | 0.19452155 | −0.0441934 | 0.11929989 |
| GPC1 | | | | | | | | | |
| SHH)- | −0.190733438 | 0.19886103 | 0.19322993 | −0.0640971 | −0.211942 | −0.1371679 | −0.0355218 | 0.06872343 | −0.3101402 |
| GPC1 | | | | | | | | | |
| VEGFA)- | −0.110082216 | 0.26225904 | 0.24433501 | 0.34878813 | 0.23476904 | −0.0680923 | −0.271745 | 0.0597239 | −0.3223557 |
| GPC1 | | | | | | | | | |
| CD274)- | −0.083908014 | 0.03370711 | 0.18448114 | 0.18603411 | −0.1103839 | 0.24980585 | −0.1551726 | 0.26677274 | 0.59528291 |
| CD80 | | | | | | | | | |
| IL18)- | −0.124836058 | −0.1293216 | 0.18658277 | 0.01542746 | 0.11606902 | −0.0220204 | 0.05973079 | 0.04693305 | 0.09773545 |
| IL18RAP | | | | | | | | | |
| RTN4)- | −0.20172978 | −0.0255089 | 0.01847223 | −0.0958863 | 0.034603 | −0.1060537 | −0.1200855 | −0.0427341 | 0.01085901 |
| CNTNAP1 | | | | | | | | | |
| TNC)- | 0.048815762 | −0.0710277 | 0.10683259 | 0.01090341 | −0.0657742 | −0.0064098 | 0.07323004 | −0.0898854 | 0.18790745 |
| ITGA9 | | | | | | | | | |
| VEGFC)- | −0.051575818 | 0.14743013 | 0.15353602 | 0.1037904 | −0.0170678 | 0.104596 | 0.09329628 | 0.02026289 | 0.47598459 |
| ITGA9 | | | | | | | | | |
| SPP1)- | 0.039052428 | −0.209698 | −0.1557843 | 0.1074099 | 0.01799044 | −0.1067893 | 0.05057866 | −0.1009638 | 0.14866306 |
| ITGA9 | | | | | | | | | |
| F13A1)- | 0.200082637 | −0.0356467 | 0.01920615 | 0.01055245 | 0.18037244 | 0.01880841 | 0.1429036 | 0.05160567 | 0.3699211 |
| ITGA9 | | | | | | | | | |
| ADAM12)- | −0.084327077 | −0.0051561 | 0.09534322 | 0.03467666 | 0.01734993 | 0.11868239 | 0.07661716 | −0.0336564 | 0.30139466 |
| ITGA9 | | | | | | | | | |
| VCAM1)- | −0.140775988 | 0.03824172 | 0.14667546 | 0.11594195 | 0.16192523 | 0.26440443 | 0.2141588 | −0.1276947 | 0.46910152 |
| ITGA9 | | | | | | | | | |
| TGM2)- | −0.209217734 | 0.00079385 | 0.00259267 | −0.1215161 | −0.1080757 | −0.0547153 | 0.00251062 | −0.0547334 | 0.23460349 |
| ITGA9 | | | | | | | | | |
| VEGFA)- | −0.200811056 | 0.14230245 | −0.0403155 | 0.17395049 | −0.006845 | −0.1854858 | −0.0502272 | 0.23917637 | −0.2986858 |
| ITGA9 | | | | | | | | | |
| CSF2)- | 0.03870028 | 0.09688329 | 0.0557098 | 0.11285536 | −0.0263019 | 0.14467474 | 0.01322113 | 0.20322103 | 0.35194244 |
| ITGA9 | | | | | | | | | |
| ADAM15)- | −0.250750107 | 0.0970667 | 0.17013479 | 0.2583548 | 0.10253143 | −0.0100091 | −0.003284 | 0.0570013 | 0.21281519 |
| ITGA9 | | | | | | | | | |
| FN1)- | −0.128327631 | −0.1348249 | 0.10767263 | −0.117461 | −0.0828557 | 0.15120976 | 0.07532163 | −0.1834625 | −0.0040659 |
| ITGA9 | | | | | | | | | |
| NPPC)- | 0.026177014 | 0.08037971 | 0.16022111 | 0.1168655 | 0.08115801 | −0.1927134 | 0.36056968 | −0.2462151 | 0.5136071 |
| NPR3 | | | | | | | | | |
| VEGFB)- | −0.132155047 | −0.0039301 | 0.15387771 | −0.1888672 | −0.0964663 | 0.19592501 | 0.05895321 | −0.1301597 | 0.02842345 |
| NRP1 | | | | | | | | | |
| SEMA3D)- | −0.063091117 | 0.1278078 | −0.0001246 | −0.0915045 | −0.2383099 | 0.10204396 | −0.2044416 | 0.0383154 | 0.11896044 |
| NRP1 | | | | | | | | | |
| SEMA3B)- | 0.101994952 | 0.07888881 | −0.1260626 | 0.13959477 | −0.0061173 | −0.0571738 | 0.08577434 | −0.0739346 | −0.0388096 |
| NRP1 | | | | | | | | | |
| PGF)- | −0.090770947 | 0.121285 | 0.08838232 | 0.06418965 | −0.1630384 | 0.07487392 | −0.1350413 | −0.0062311 | 0.16924228 |
| NRP1 | | | | | | | | | |
| SEMA3E)- | 0.030225135 | 0.12534753 | −0.0955943 | −0.0540387 | −0.0842847 | −0.0779066 | −0.1590191 | 0.08679922 | 0.28427619 |
| NRP1 | | | | | | | | | |
| SEMA4A)- | 0.001559784 | −0.0019305 | 0.10724168 | 0.02497202 | −0.0731468 | 0.02028921 | −0.013072 | −0.0621091 | −0.1005715 |
| NRP1 | | | | | | | | | |
| VEGFA)- | −0.126161756 | 0.23055888 | 0.10854114 | 0.22860399 | −0.0190867 | −0.0609142 | 0.01652897 | 0.09715206 | −0.2163606 |
| NRP1 | | | | | | | | | |
| FGF7)- | 0.015596421 | −0.0380526 | −0.0682815 | 0.03313143 | 0.02911636 | 0.04459816 | −0.1280922 | 0.01811036 | 0.17215404 |
| NRP1 | | | | | | | | | |
| NLGN2)- | −0.169323185 | −0.0746243 | 0.11678743 | −0.2390572 | −0.2105066 | 0.25439653 | −0.1292247 | 0.00208895 | 0.17280092 |
| NRXN2 | | | | | | | | | |
| AFDN)- | −0.01076299 | 0.13463048 | 0.05334982 | −0.0329027 | −0.1083953 | −0.0035451 | 0.16373584 | −0.1374858 | 0.10060505 |
| NRXN2 | | | | | | | | | |
| CALM2)- | −0.204628681 | −0.0877056 | 0.10368989 | −0.2590502 | 0.01925815 | 0.00125824 | −0.0127391 | 0.23964846 | −0.0800205 |
| CACNA1C | | | | | | | | | |
| NCAM1)- | −0.036971811 | 0.15967301 | 0.15491573 | −0.1229916 | −0.014335 | 0.12246294 | 0.1117985 | −0.0715879 | 0.23309591 |
| CACNA1C | | | | | | | | | |
| CALM1)- | 0.040396405 | −0.0357061 | −0.0127418 | −0.0175362 | −0.1480819 | 0.05402598 | −0.1596286 | −0.117961 | 0.13351446 |
| CACNA1C | | | | | | | | | |
| CALM3)- | −0.090806152 | 0.04182138 | −0.1430122 | 0.09006022 | −0.0057531 | −0.0098422 | −0.0243884 | 0.19453083 | −0.2991802 |
| CACNA1C | | | | | | | | | |
| RSPO3)- | −0.011945583 | 0.09811754 | 0.29885004 | −0.0205438 | −0.0540266 | 0.19078544 | 0.10578708 | 0.16635954 | 0.37633908 |
| LGR5 | | | | | | | | | |
| VCAM1)- | −0.039736391 | 0.00972969 | 0.0327722 | 0.07147451 | 0.02438682 | −0.0323752 | 0.2015191 | −0.1088349 | 0.23658182 |
| ITGB7 | | | | | | | | | |
| FN1)- | 0.031878276 | −0.0186511 | −0.0761271 | −0.1841703 | 0.02228712 | −0.0763846 | −0.1542919 | 0.04227489 | −0.0151827 |
| ITGB7 | | | | | | | | | |
| NRG3)- | 0.031592213 | −0.1414217 | 0.09012777 | −0.1573723 | 0.01850471 | 0.08380565 | −0.0196206 | 0.0305416 | 0.13680557 |
| ERBB3 | | | | | | | | | |
| NRG1)- | 0.235301753 | −0.143147 | 0.27062149 | −0.1510709 | 0.09602803 | 0.10231987 | 0.45486848 | 0.3768424 | −0.4366091 |
| ERBB3 | | | | | | | | | |
| AREG)- | 0.013186775 | 0.04582051 | 0.01076318 | −0.0793235 | 0.03887218 | 0.04342241 | 0.31521247 | 0.25638641 | −0.4103827 |
| ERBB3 | | | | | | | | | |
| L1CAM)- | 0.132157486 | −0.153624 | 0.11763821 | −0.2104968 | −0.0072863 | 0.02820822 | 0.43789024 | 0.31465812 | −0.5530428 |
| ERBB3 | | | | | | | | | |
| TGFA)- | 0.303697543 | −0.183729 | 0.30138445 | −0.2626846 | 0.28446665 | 0.34008497 | −0.0113777 | −0.0888697 | −0.1287403 |
| ERBB3 | | | | | | | | | |
| BTC)- | 0.282151524 | −0.2394116 | 0.2809619 | −0.1052818 | 0.08072649 | 0.0755769 | 0.31642575 | 0.32456525 | −0.322446 |
| ERBB3 | | | | | | | | | |
| SST)- | 0.10817297 | −0.023466 | 0.24679451 | 0.12014695 | 0.04987896 | 0.26513487 | 0.09234899 | −0.0365902 | 0.29960308 |
| SSTR1 | | | | | | | | | |
| GPC3)- | −0.131530835 | −0.0431448 | 0.27375134 | 0.02693628 | 0.06452924 | 0.07656626 | 0.0091651 | −0.1887646 | 0.13728638 |
| IGF1R | | | | | | | | | |
| INS)- | −0.100350421 | −0.0835886 | 0.10618743 | 0.02185431 | −0.0380979 | 0.02868833 | −0.1491072 | −0.0019269 | 0.02945015 |
| IGF1R | | | | | | | | | |
| IGF2)- | −0.270264272 | 0.01536792 | 0.0837307 | −0.1797814 | −0.0072159 | 0.21637711 | 0.12334836 | −0.2049853 | −0.0077569 |
| IGF1R | | | | | | | | | |
| GNAI2)- | 0.040484302 | −0.2193066 | 0.02923328 | −0.1539676 | 0.04003783 | 0.12541452 | 0.02195342 | −0.024585 | −0.1072957 |
| IGF1R | | | | | | | | | |
| IGF1)- | −0.201039028 | 0.04731888 | 0.1398393 | 0.20091639 | −0.123057 | 0.16237973 | 0.08908457 | −0.0912788 | 0.13184074 |
| IGF1R | | | | | | | | | |
| HSP90B1)- | −0.260910202 | −0.2720572 | −0.2600385 | −0.1242574 | −0.0177618 | −0.1948027 | 0.09904562 | 0.06294156 | −0.0348913 |
| ASGR1 | | | | | | | | | |
| S100A4)- | 0.006220605 | 0.09663453 | 0.03441584 | 0.09328043 | 0.2117941 | 0.24535805 | −0.0527602 | −0.0094003 | −0.0229019 |
| ERBB2 | | | | | | | | | |
| NRG3)- | 0.186323747 | −0.3268479 | 0.19746 | −0.2013217 | −0.0357937 | 0.05226126 | 0.04200372 | 0.064115 | 0.08581623 |
| ERBB2 | | | | | | | | | |
| SEMA4D)- | −0.2477604 | 0.13215441 | −0.2151189 | −0.2092699 | 0.19491693 | 0.22554597 | 0.11971896 | 0.14984284 | 0.0108475 |
| ERBB2 | | | | | | | | | |
| NRG1)- | 0.151655264 | −0.194216 | 0.21009006 | −0.1816718 | 0.03275703 | 0.04150156 | 0.3355694 | 0.28776255 | −0.6023133 |
| ERBB2 | | | | | | | | | |
| NRG4)- | 0.145001669 | −0.1627116 | 0.03295319 | −0.2453797 | 0.12460571 | 0.05154878 | 0.1834911 | 0.2124967 | −0.3446017 |
| ERBB2 | | | | | | | | | |
| HSP90B1)- | −0.199175619 | 0.31095373 | −0.1283783 | 0.21324473 | 0.0979285 | 0.11171961 | 0.19642679 | 0.15750922 | 0.03371723 |
| ERBB2 | | | | | | | | | |
| L1CAM)- | 0.179153099 | −0.1940148 | 0.16047831 | −0.2850396 | 0.03813104 | 0.08697716 | 0.17212768 | 0.20386032 | −0.5213567 |
| ERBB2 | | | | | | | | | |
| TGFA)- | 0.185707339 | −0.2248213 | 0.30530388 | −0.2049288 | 0.13093965 | 0.19338286 | −0.1932747 | −0.1289615 | 0.0415144 |
| ERBB2 | | | | | | | | | |
| HSP90AA1)- | 0.261779706 | −0.002821 | 0.2736379 | −0.0007294 | 0.27694219 | 0.31064443 | −0.0131381 | −0.0501596 | 0.36428962 |
| ERBB2 | | | | | | | | | |
| BTC)- | 0.126053082 | −0.2653344 | 0.19895579 | −0.1270134 | 0.07311346 | 0.17417967 | 0.18874872 | 0.30426428 | −0.4577246 |
| ERBB2 | | | | | | | | | |
| IFNA16)- | 0.004669556 | 0.03222026 | 0.09625369 | −0.0192746 | −0.1086504 | 0.22787371 | 0.09858299 | 0.17106809 | 0.06997594 |
| IFNAR1 | | | | | | | | | |
| IFNA14)- | −0.10966843 | 0.06301051 | 0.05330095 | −0.1169292 | −0.1432352 | 0.21615204 | 0.0483995 | −0.0664038 | −0.141975 |
| IFNAR1 | | | | | | | | | |
| IFNA4)- | 0.236912699 | 0.05217238 | −0.1078924 | −0.0621386 | −0.0244224 | 0.03299941 | 0.18417911 | −0.0478579 | −0.2132861 |
| IFNAR1 | | | | | | | | | |
| IFNA13)- | −0.089529215 | −0.0586769 | 0.0193879 | −0.1134655 | 0.08742718 | 0.06042729 | 0.02320304 | 0.06169377 | −0.149216 |
| IFNAR1 | | | | | | | | | |
| IFNB1)- | −0.087388156 | 0.08435691 | 0.12136378 | −0.2388451 | −0.0648188 | 0.23580888 | 0.09412378 | 0.12200945 | 0.03397735 |
| IFNAR1 | | | | | | | | | |
| IFNA8)- | 0.100126879 | 0.07438516 | −0.0393185 | −0.0912658 | −0.0957312 | −0.0872712 | 0.15774216 | 0.10058383 | −0.0737841 |
| IFNAR1 | | | | | | | | | |
| IFNE)- | 0.215510942 | −0.0991424 | −0.0207651 | −0.0885819 | −0.0433855 | 0.15939165 | 0.10144588 | −0.0238659 | −0.0194084 |
| IFNAR1 | | | | | | | | | |
| MMP9)- | 0.030697376 | 0.01178331 | 0.02848264 | 0.00014647 | −0.0255608 | 0.19489388 | 0.03089215 | 0.07393509 | 0.03770639 |
| IFNAR1 | | | | | | | | | |
| IFNA2)- | 0.036057101 | 0.01132876 | −0.0416421 | −0.06333 | −0.1150794 | 0.06422792 | 0.11126656 | 0.21987544 | 0.00106335 |
| IFNAR1 | | | | | | | | | |
| IL24)- | −0.006174114 | 0.01901106 | 0.16607973 | 0.04953063 | 0.10964218 | 0.13073633 | 0.07358981 | 0.14634211 | −0.0872416 |
| IL22RA1 | | | | | | | | | |
| TNF)- | −0.107939114 | 0.11986199 | 0.04919667 | 0.22042249 | −0.0436976 | 0.14158931 | −0.0960705 | 0.02033143 | 0.15526148 |
| CELSR2 | | | | | | | | | |
| PTPN11)- | −0.05678117 | −0.0112754 | 0.07121445 | 0.02037337 | −0.0950646 | −0.1007655 | 0.03039516 | 0.11271196 | 0.13507952 |
| FCRL4 | | | | | | | | | |
| PTPN6)- | 0.017091851 | −0.0403334 | 0.05663742 | 0.13644 | −0.010454 | 0.08483836 | 0.08169612 | 0.10716557 | 0.20290972 |
| FCRL4 | | | | | | | | | |
| TFF1)- | 0.143091534 | 0.09383271 | 0.17369202 | 0.19041286 | 0.20616232 | 0.15671516 | −0.0258821 | −0.0025519 | 0.04115135 |
| FCRL4 | | | | | | | | | |
| CXCL12)- | −0.083967903 | 0.05492501 | 0.13066096 | −0.0392465 | −0.1487685 | 0.19268425 | 0.17298906 | −0.0327429 | 0.05958843 |
| ACKR3 | | | | | | | | | |
| CXCL11)- | −0.085402442 | 0.1906166 | 0.24650613 | 0.06079382 | −0.0557006 | 0.08051678 | 0.14930555 | 0.06238445 | 0.27584035 |
| ACKR3 | | | | | | | | | |
| IL31)- | −0.052184327 | −0.0136736 | −0.0569343 | 0.07233538 | 0.08753715 | −0.0036408 | 0.08753556 | 0.03964914 | −0.1075046 |
| OSMR | | | | | | | | | |
| S100A4)- | −0.105383279 | 0.08427929 | 0.02557931 | 0.14630167 | −0.0130268 | −0.1031625 | 0.02785818 | 0.11628888 | −0.0758355 |
| EGFR | | | | | | | | | |
| NRG1)- | 0.026551906 | −0.0013381 | 0.19339924 | −0.0110866 | −0.1081378 | 0.12462915 | 0.23405306 | −0.0260642 | 0.01466739 |
| EGFR | | | | | | | | | |
| AREG)- | −0.070274597 | 0.00965272 | 0.06849317 | 0.11730817 | −0.0999634 | −0.1341483 | 0.18047666 | 0.03049688 | −0.0039078 |
| EGFR | | | | | | | | | |
| NRG4)- | 0.040534423 | −0.0172782 | 0.06315617 | 0.09002003 | −0.0104093 | −0.0357839 | 0.0944512 | −0.0292767 | 0.07853953 |
| EGFR | | | | | | | | | |
| ANXA1)- | 0.115626658 | −0.2658111 | 0.12378857 | −0.3059903 | 0.06166568 | 0.02043492 | −0.05794 | −0.0246681 | 0.19889241 |
| EGFR | | | | | | | | | |
| L1CAM)- | −0.064171066 | 0.14029722 | 0.11445307 | −0.1649981 | 0.13991992 | 0.2216497 | 0.11673835 | 0.03028439 | 0.08345771 |
| EGFR | | | | | | | | | |
| EFEMP1)- | 0.092066599 | 0.0138765 | 0.21824372 | −0.137156 | −0.1006712 | 0.02906604 | 0.20001699 | 0.08733165 | −0.0082052 |
| EGFR | | | | | | | | | |
| FGL1)- | 0.137174949 | −0.1173858 | 0.18882579 | −0.0044623 | −0.1594075 | −0.034186 | 0.21688486 | 0.09934213 | −0.0420365 |
| EGFR | | | | | | | | | |
| TGFA)- | 0.110585967 | −0.0068718 | 0.20680688 | 0.03450231 | −0.1636963 | 0.03579554 | 0.08998317 | −0.0658082 | −0.008261 |
| EGFR | | | | | | | | | |
| ARF4)- | −0.181121176 | 0.05242421 | −0.060927 | 0.10056508 | −0.0312438 | 0.0362395 | 0.01500574 | −0.0535521 | −0.0478571 |
| EGFR | | | | | | | | | |
| GNAI2)- | 0.072030366 | 0.00740464 | −0.0957954 | −0.0696378 | 0.07890851 | 0.10957528 | 0.14364732 | 0.05428455 | −0.1074024 |
| EGFR | | | | | | | | | |
| ANG)- | −0.045407855 | 0.07682751 | −0.0473429 | 0.21093198 | −0.0005508 | −0.0263912 | −0.0114639 | 0.07656126 | −0.1337005 |
| EGFR | | | | | | | | | |
| GSTP1)- | 0.034889887 | −0.1231126 | 0.13339642 | −0.1462546 | 0.19841154 | 0.07979937 | −0.0164018 | 0.05540085 | 0.06529865 |
| EGFR | | | | | | | | | |
| HSP90AA1)- | 0.048469082 | 0.02303948 | 0.10428434 | −0.0253976 | 0.12813181 | 0.07096924 | −0.1265221 | −0.0052881 | −0.0137798 |
| EGFR | | | | | | | | | |
| CNTF)- | −0.108192549 | −0.0535362 | 0.03827538 | 0.10777005 | 0.04842392 | −0.0094651 | 0.13210043 | −0.052502 | 0.09628863 |
| EGFR | | | | | | | | | |
| BTC)- | −0.020243842 | 0.09425273 | 0.09541271 | 0.11943172 | −0.0812603 | 0.0435654 | 0.0889827 | 0.04703353 | −0.0106483 |
| EGFR | | | | | | | | | |
| CCN2)- | −0.024966968 | −0.1595305 | 0.21575222 | 0.04355748 | −0.1139131 | −0.0839653 | 0.22527363 | 0.05155113 | −0.2185162 |
| EGFR | | | | | | | | | |
| LRIG2)- | 0.063797611 | 0.05068713 | 0.15664329 | 0.00310238 | −0.0682489 | −0.1658344 | 0.05563913 | −0.0796608 | 0.05302049 |
| EGFR | | | | | | | | | |
| EPGN)- | 0.015221799 | −0.0431506 | 0.26197003 | 0.02103541 | 0.00071497 | −0.0421533 | 0.15343893 | −0.0172766 | −0.0556773 |
| EGFR | | | | | | | | | |
| VEGFA)- | 0.152001091 | −0.1215557 | 0.2735127 | −0.0495595 | 0.18968427 | 0.12211275 | −0.3276 | −0.0562768 | 0.23078478 |
| EGFR | | | | | | | | | |
| CALM1)- | −0.045158483 | 0.06735416 | −0.1040146 | 0.0269931 | −0.080841 | 3.52E−05 | −0.0659146 | −0.0725104 | −0.2386765 |
| EGFR | | | | | | | | | |
| SPINK1)- | 0.061254985 | 0.09436195 | 0.14264201 | 0.05347232 | 0.16101181 | −0.04576 | −0.1225275 | 0.00515336 | −0.0582242 |
| EGFR | | | | | | | | | |
| CALM3)- | −0.04903285 | 0.1711945 | −0.0191992 | 0.12993199 | 0.1597631 | 0.10370595 | −0.0227387 | 0.03037823 | −0.0460791 |
| EGFR | | | | | | | | | |
| EFNA3)- | 0.002554813 | 0.08442421 | 0.10330261 | 0.00894713 | −0.0866447 | 0.1265494 | 0.10006111 | −0.0632014 | −0.2024492 |
| EPHA1 | | | | | | | | | |
| EFNA1)- | 0.232163863 | 0.03116528 | 0.01051427 | 0.0050502 | −0.0280118 | 0.28389078 | −0.1716684 | −0.1850019 | −0.1789342 |
| EPHA1 | | | | | | | | | |
| EFNA4)- | 0.074511114 | 0.02129056 | 0.19606097 | −0.0333665 | −0.0403438 | 0.05528476 | −0.0760729 | −0.1135357 | −0.1162125 |
| EPHA1 | | | | | | | | | |
| LIN7C)- | −0.038064063 | −0.0080687 | 0.14581096 | 0.00482178 | 0.04981466 | −0.0387427 | −0.1485091 | 0.02842685 | −0.0129564 |
| HTR2C | | | | | | | | | |
| CALM1)- | −0.100672499 | −0.0299054 | 0.04310289 | 0.04318416 | −0.0143858 | 0.0084668 | −0.2376528 | 0.04375362 | −0.0445404 |
| HTR2C | | | | | | | | | |
| FASLG)- | −0.031934789 | 0.01987184 | −0.0798083 | −0.0168407 | −0.0146762 | 0.12386093 | −0.0419358 | 0.08924096 | −0.1294885 |
| TNFRSF10B | | | | | | | | | |
| NTF4)- | −0.022825528 | 0.19731026 | 0.15996723 | 0.1761409 | 0.09458172 | 0.24346391 | 0.07475009 | 0.14962141 | 0.31010425 |
| NTRK2 | | | | | | | | | |
| BDNF)- | 0.024357183 | 0.20581062 | 0.31823327 | 0.19106687 | −0.0808787 | 0.29786004 | 0.08899726 | 0.23806176 | 0.44760616 |
| NTRK2 | | | | | | | | | |
| DLK1)- | 0.090107352 | −0.0075598 | −0.0115441 | −0.060974 | 0.05504778 | 0.12531193 | 0.13609544 | 0.16167705 | 0.00602275 |
| NOTCH1 | | | | | | | | | |
| DLL1)- | 0.020592995 | −0.0058198 | 0.16091316 | −0.1089001 | −0.106851 | 0.08209777 | −0.1202914 | 0.09315075 | −0.076462 |
| NOTCH1 | | | | | | | | | |
| MFNG)- | 0.065640711 | 0.02709983 | 0.08320364 | −0.0747765 | −0.0410981 | 0.02584891 | −0.1508272 | 0.17000192 | −0.030694 |
| NOTCH1 | | | | | | | | | |
| PSEN1)- | −0.085476062 | −0.0536788 | 0.12201478 | −0.0214523 | 0.2010597 | −0.0127581 | −0.2114381 | 0.03850508 | −0.046164 |
| NOTCH1 | | | | | | | | | |
| TNF)- | 0.140575229 | −0.0393268 | 0.0967654 | 0.0546884 | 0.09186526 | 0.02243641 | −0.1160387 | 0.16858412 | −0.0145266 |
| NOTCH1 | | | | | | | | | |
| WNT4)- | −0.005262957 | −0.0228882 | 0.12991197 | −0.0095202 | 0.04674352 | 0.04516875 | −0.0210646 | 0.06139399 | 0.05018763 |
| NOTCH1 | | | | | | | | | |
| DLK2)- | 0.073997509 | −0.0023779 | 0.10505933 | 0.02295257 | 0.05404602 | 0.17148474 | 0.13025951 | −0.0048463 | −0.0319709 |
| NOTCH1 | | | | | | | | | |
| ADAM17)- | 0.085343243 | −0.1105916 | 0.17030078 | −0.1088991 | 0.09852341 | 0.13257581 | −0.0395056 | 0.07123164 | 0.10892596 |
| NOTCH1 | | | | | | | | | |
| MFAP5)- | 0.140178543 | 0.03244684 | 0.19487291 | −0.0526131 | 0.01562836 | −0.1308656 | 0.12636285 | 0.14235334 | 0.08791264 |
| NOTCH1 | | | | | | | | | |
| MFAP2)- | −0.105877872 | −0.0341666 | 0.0416309 | −0.1733503 | 0.04265129 | −0.0453139 | 0.08190499 | 0.01020851 | 0.06535373 |
| NOTCH1 | | | | | | | | | |
| RBP3)- | 0.020734798 | 0.07379192 | 0.01595241 | −0.0651266 | 0.16568379 | 0.06234533 | 0.09493062 | 0.08893042 | 0.17102457 |
| NOTCH1 | | | | | | | | | |
| JAG2)- | 0.108841115 | 0.06929085 | 0.14776916 | −0.0559988 | 0.13385943 | 0.19047534 | 0.02361324 | 0.08557924 | 0.41155043 |
| NOTCH1 | | | | | | | | | |
| PLAU)- | −0.054115335 | 0.12406661 | −0.0721514 | 0.08924504 | −0.0201837 | −0.02599 | 0.06722802 | 0.10975954 | 0.00183669 |
| ST14 | | | | | | | | | |
| SPINT1)- | 0.503400962 | −0.1991414 | 0.45987416 | −0.1979111 | 0.33339778 | 0.53223151 | −0.4136659 | −0.3875766 | 0.41820701 |
| ST14 | | | | | | | | | |
| GNAI2)- | −0.248970594 | 0.17699275 | −0.0338504 | 0.05250007 | −0.0139794 | −0.0687946 | −0.0710053 | −0.1142726 | −0.2315196 |
| ADRA2A | | | | | | | | | |
| AGT)- | 0.04966707 | −0.0995313 | 0.01565972 | −0.1272128 | 0.13510908 | 0.09041868 | −0.0079513 | 0.16547709 | −0.1203224 |
| ADRA2A | | | | | | | | | |
| CXCL12)- | −0.109464249 | −0.0479467 | 0.13407575 | 0.01555391 | −0.1344495 | 0.14655809 | 0.18429622 | 0.10634889 | 0.15103083 |
| ADRA2A | | | | | | | | | |
| CXCL3)- | −0.00332367 | −0.008238 | 0.1302 | −0.2305228 | 0.175833 | 0.06562392 | −0.1083049 | −0.0877961 | 0.34505759 |
| ADRA2A | | | | | | | | | |
| CCL5)- | −0.176186726 | 0.23420763 | −0.2016158 | −0.1883426 | 0.07827753 | 0.10209145 | 0.10933536 | 0.13988444 | −0.1688292 |
| ADRA2A | | | | | | | | | |
| CXCL11)- | 0.022082432 | −0.0169491 | 0.09456446 | −0.044825 | 0.01663488 | 0.2128185 | 0.06025773 | 0.15550126 | −0.0934605 |
| ADRA2A | | | | | | | | | |
| NMS)- | 0.042662836 | −0.0902074 | 0.15834076 | −0.0962882 | 0.02135223 | 0.09432547 | 0.2313038 | 0.12400126 | −0.1126681 |
| ADRA2A | | | | | | | | | |
| APLN)- | 0.038124612 | −0.0095159 | 0.10921221 | −0.0077635 | −0.0870893 | −0.0337909 | 0.05608714 | 0.02665749 | −0.0883194 |
| ADRA2A | | | | | | | | | |
| SST)- | −0.043775441 | −0.0835243 | 0.1849161 | 0.02417881 | −0.1448927 | 0.15946678 | 0.00643079 | 0.12730543 | −0.0924291 |
| ADRA2A | | | | | | | | | |
| NPY)- | 0.079856428 | −0.0177867 | 0.05192588 | −0.1427789 | 0.01625189 | 0.12268923 | 0.24343554 | 0.20722212 | −0.1393072 |
| ADRA2A | | | | | | | | | |
| POMC)- | 0.09651139 | 0.01767084 | 0.14871039 | −0.0683342 | −0.0445989 | 0.06031804 | −0.0145491 | 0.13457344 | 0.00346833 |
| ADRA2A | | | | | | | | | |
| CXCL8)- | −0.014487319 | 0.07531933 | −0.0844322 | −0.1069926 | 0.05457033 | 0.07193379 | 0.20082531 | 0.11771168 | −0.1817386 |
| ADRA2A | | | | | | | | | |
| CXCL2)- | 0.016359798 | −0.1887696 | 0.29800195 | −0.1220589 | 0.20420897 | 0.11967662 | 0.0317351 | −0.0468425 | 0.07316609 |
| ADRA2A | | | | | | | | | |
| CXCL13)- | 0.026840568 | −0.1752351 | 0.08321104 | −0.0335725 | −0.1085212 | 0.06498293 | 0.08693302 | 0.07551686 | 0.02565511 |
| ADRA2A | | | | | | | | | |
| CXCL10)- | 0.047321281 | 0.06197287 | 0.03234107 | 0.00024026 | −0.0472083 | 0.01524974 | 0.1354158 | 0.10913442 | −0.0740124 |
| ADRA2A | | | | | | | | | |
| PPBP)- | 0.01806763 | 0.03933622 | 0.13728965 | 0.05440064 | 0.0458785 | −0.0224311 | 0.15706099 | 0.12484053 | −0.071467 |
| ADRA2A | | | | | | | | | |
| CXCL5)- | 0.072348734 | 0.00620446 | 0.22492595 | −0.0221377 | 0.15910499 | 0.07935827 | −0.1416622 | −0.2937421 | 0.06630841 |
| ADRA2A | | | | | | | | | |
| CCL19)- | 0.137351807 | 0.09190883 | 0.17262829 | −0.1473686 | −0.1050995 | 0.09898764 | 0.01342853 | −0.0215677 | −0.0666981 |
| ADRA2A | | | | | | | | | |
| APP)- | −0.069143101 | 0.12470871 | 0.10595243 | 0.43592658 | −0.0935383 | −0.2240638 | 0.06995062 | −0.0282042 | −0.2837554 |
| ADRA2A | | | | | | | | | |
| CXCL1)- | −0.068501059 | −0.0205303 | 0.11381016 | −0.3408487 | 0.12445777 | 0.15229919 | 0.08938656 | 0.03203288 | −0.0082343 |
| ADRA2A | | | | | | | | | |
| NPB)- | 0.197552298 | 0.01083592 | 0.08013479 | −0.1029329 | −0.0354123 | 0.04365457 | 0.05261847 | 0.11109321 | −0.1569762 |
| ADRA2A | | | | | | | | | |
| PPY)- | −0.196380089 | 0.08892141 | −0.0037277 | 0.12285619 | −0.133338 | 0.12252908 | 0.04621089 | −0.0997076 | 0.16529037 |
| ADRA2A | | | | | | | | | |
| CXCL9)- | 0.149641333 | 0.02388919 | 0.05550507 | −0.0192002 | 0.06714422 | −0.0662102 | 0.13832504 | 0.14022404 | −0.1041758 |
| ADRA2A | | | | | | | | | |
| CXCL16)- | −0.062070077 | 0.13560395 | −0.0379228 | −0.040244 | 0.04901134 | 0.13347912 | −0.1637232 | 0.02199096 | 0.19418264 |
| ADRA2A | | | | | | | | | |
| CCL20)- | 0.091556806 | −0.0901458 | 0.02309855 | −0.1396243 | −0.0369348 | 0.1237407 | 0.06630523 | 0.00925404 | 0.0340128 |
| ADRA2A | | | | | | | | | |
| NPW)- | 0.212975765 | −0.0337683 | 0.09750512 | −0.0303111 | −0.0214277 | −0.0259182 | 0.08032485 | 0.10385859 | −0.0982497 |
| ADRA2A | | | | | | | | | |
| PENK)- | −0.095952851 | −0.0498412 | 0.02963203 | −0.1386143 | 0.0241925 | 0.13594082 | 0.07004447 | 0.07254352 | −0.0298763 |
| ADRA2A | | | | | | | | | |
| CCL28)- | 0.017725307 | 0.0339291 | 0.15480538 | −0.1374101 | 0.20459144 | 0.00071632 | −0.2178551 | 0.01255394 | −0.0217928 |
| ADRA2A | | | | | | | | | |
| PF4)- | 0.005024692 | 0.00466705 | −0.0510811 | −0.1286739 | 0.098731 | 0.20884621 | 0.15374159 | 0.1160421 | −0.1417021 |
| ADRA2A | | | | | | | | | |
| ANXA1)- | −0.172701281 | 0.36258499 | −0.0211442 | 0.23078669 | −0.0521691 | −0.0401667 | 0.06283427 | −0.0148403 | −0.2779231 |
| ADRA2A | | | | | | | | | |
| EFNA3)- | 0.105211193 | −0.07232 | 0.30133908 | −0.1342006 | 0.0690372 | 0.17645646 | 0.02038745 | −0.0794653 | 0.28884619 |
| EPHA4 | | | | | | | | | |
| EFNA1)- | 0.086468743 | −0.0615298 | 0.32575479 | −0.0708967 | 0.2213775 | 0.12147348 | −0.3131421 | −0.1140845 | 0.05741399 |
| EPHA4 | | | | | | | | | |
| EFNA4)- | 0.020499944 | 0.02410164 | 0.14458933 | −0.1369168 | 0.05292452 | 0.31557643 | −0.2009184 | −0.0750211 | 0.24435806 |
| EPHA4 | | | | | | | | | |
| PDGFC)- | 0.298397874 | 0.01082011 | −0.1434547 | −0.157411 | 0.02274773 | −0.2054263 | −0.2137842 | 0.32005192 | 0.1817295 |
| FLT1 | | | | | | | | | |
| VEGFC)- | −0.039869719 | −0.0399156 | 0.06773299 | 0.00204669 | −0.0967985 | 0.10753185 | −0.1893717 | 0.13108273 | 0.16284193 |
| FLT1 | | | | | | | | | |
| PGF)- FLT1 | −0.10324527 | 0.03106523 | 0.144731 | 0.08382647 | −0.393668 | 0.24782576 | −0.1491488 | 0.04071887 | 0.18065471 |
| VEGFB)- | 0.048590671 | −0.0241989 | 0.10877542 | −0.2417081 | 0.06479908 | −0.2467503 | −0.0022218 | 0.11036742 | 0.16433906 |
| FLT1 | | | | | | | | | |
| VEGFA)- | −0.079162175 | 0.16417915 | −0.0490192 | 0.10642334 | −0.1122761 | −0.0772765 | 0.23859064 | −0.124291 | −0.1314302 |
| FLT1 | | | | | | | | | |
| SORBS1)- | −0.120325508 | 0.13837593 | 0.09751292 | −0.1711465 | −0.1972655 | 0.18304327 | 0.14441899 | −0.1497963 | 0.30774909 |
| ITGA1 | | | | | | | | | |
| COL4A5)- | −0.146807732 | 0.23123757 | 0.11123532 | −0.1432148 | −0.1767384 | 0.25416931 | 0.19056345 | −0.1995778 | 0.19682021 |
| ITGA1 | | | | | | | | | |
| LAMA2)- | −0.089984366 | 0.06633297 | 0.11550914 | −0.1186787 | −0.0993047 | 0.15846663 | 0.1013319 | −0.1981098 | 0.33514024 |
| ITGA1 | | | | | | | | | |
| LAMA1)- | −0.150668068 | 0.14754739 | 0.12023289 | 0.07894749 | −0.0414703 | 0.03017567 | 0.13887111 | −0.1224193 | 0.3118398 |
| ITGA1 | | | | | | | | | |
| COL4A4)- | −0.080432933 | 0.04602172 | 0.07980961 | 0.1867513 | 0.06382474 | −0.00537 | 0.01356371 | 0.04988111 | 0.1856925 |
| ITGA1 | | | | | | | | | |
| COL5A1)- | −0.325333642 | 0.19366358 | 0.21223129 | −0.2147432 | −0.1014739 | 0.12907933 | 0.28091952 | −0.3494368 | 0.314592 |
| ITGA1 | | | | | | | | | |
| COL6A2)- | −0.253862268 | 0.16776541 | 0.17713576 | 0.04774578 | 0.06953587 | −0.032009 | 0.24140335 | −0.3299369 | 0.24806701 |
| ITGA1 | | | | | | | | | |
| COL4A3)- | 0.023004703 | 0.01668274 | 0.11045429 | 0.21988518 | 0.18660359 | −0.1314787 | 0.08107601 | −0.0866937 | 0.24732405 |
| ITGA1 | | | | | | | | | |
| COL5A2)- | −0.317601068 | 0.22668586 | 0.21453096 | −0.1867347 | −0.1256656 | 0.14320207 | 0.2457859 | −0.3335819 | 0.27690915 |
| ITGA1 | | | | | | | | | |
| COL1A2)- | −0.311872692 | 0.25088502 | 0.19371093 | −0.1806899 | −0.1214987 | 0.12413599 | 0.25028856 | −0.3397088 | 0.32566192 |
| ITGA1 | | | | | | | | | |
| MATN1)- | 0.147263002 | 0.05088548 | −0.1929267 | 0.05813156 | 0.0632848 | −0.0496952 | 0.06552738 | −0.0086981 | 0.21034053 |
| ITGA1 | | | | | | | | | |
| PLA2G10)- | 0.09972218 | 0.17621328 | 0.13625287 | 0.0635132 | 0.11039677 | −0.0207892 | −0.1623356 | 0.08331221 | 0.01956976 |
| PLA2R1 | | | | | | | | | |
| FN1)- | 0.12124461 | −0.045094 | 0.20836385 | 0.04939179 | −0.068969 | −0.1259385 | 0.35125112 | 0.24306337 | 0.00029586 |
| ITGB6 | | | | | | | | | |
| FYN)- | 0.053458365 | 0.10101813 | 0.18009415 | −0.1284099 | −0.1513544 | 0.1078623 | −0.1497501 | 0.09820312 | −0.1482268 |
| THY1 | | | | | | | | | |
| BGN)- | 0.083777517 | −0.0385617 | −0.1377915 | −0.1334265 | 0.21245116 | −0.0230436 | −0.2655088 | 0.09282364 | 0.06629064 |
| LY96 | | | | | | | | | |
| WNT11)- | −0.04201969 | 0.26043256 | 0.31833077 | 0.04136008 | −0.0463157 | −0.1025601 | 0.01360565 | −0.0068908 | 0.16651459 |
| FZD7 | | | | | | | | | |
| CSF1)- | −0.131662468 | −0.0339506 | 0.09109093 | 0.01311799 | 0.18107883 | −0.0132776 | −0.0635376 | 0.09519527 | 0.26946827 |
| CSF1R | | | | | | | | | |
| CSF2)- | 0.031773027 | 0.13445118 | 0.00196662 | 0.05471517 | 0.08660857 | 0.04301485 | 0.00172752 | −0.0143465 | 0.18698553 |
| CSF1R | | | | | | | | | |
| WNT4)- | 0.021529384 | −0.0929063 | −0.0494512 | 0.16135035 | 0.12142143 | −0.0609776 | −0.0872576 | 0.00320243 | 0.04088209 |
| FZD1 | | | | | | | | | |
| WNT7B)- | −0.110764711 | 0.18947695 | 0.19904403 | 0.19802618 | 0.05658979 | −0.0737958 | −0.0104674 | −0.0076266 | 0.06393593 |
| FZD1 | | | | | | | | | |
| B2M)- | −0.114335684 | 0.03662834 | 0.00987162 | −0.0412802 | 0.06530396 | −0.0067833 | 0.0666781 | 0.04531067 | −0.1395093 |
| CD1A | | | | | | | | | |
| IFNL1)- | −0.1069763 | −0.0131189 | 0.20028621 | 0.09966911 | 0.06812261 | 0.23921177 | −0.0105644 | 0.17180869 | 0.05323803 |
| IL10RB | | | | | | | | | |
| IL10)- | 0.002337524 | −0.041131 | 0.16169412 | 0.12861855 | 0.06059229 | 0.06514141 | −0.011064 | 0.24369176 | 0.13026915 |
| IL10RB | | | | | | | | | |
| IL24)- | 0.025552436 | 0.007605 | 0.05102089 | 0.06627803 | −0.0908542 | 0.14749744 | 0.07555906 | 0.08945393 | 0.03682173 |
| IL10RB | | | | | | | | | |
| UCN3)- | 0.167723321 | −0.0578588 | 0.01922431 | 0.10563587 | 0.09771292 | −0.0227862 | 0.0411423 | 0.13496495 | 0.00028415 |
| IL10RB | | | | | | | | | |
| PENK)- | −0.022040348 | 0.08976555 | −0.0212624 | 0.04125755 | −0.1625235 | −0.1101789 | −0.0661796 | −0.0254752 | −0.2126143 |
| OGFR | | | | | | | | | |
| GPI)- | −0.009173425 | −0.0668471 | 0.1088077 | 0.09773131 | −0.0503715 | 0.07544193 | 0.08389834 | 0.07148905 | 0.31452893 |
| AMFR | | | | | | | | | |
| TNFSF13B)- | 0.072573957 | 0.16895023 | 0.03972997 | 0.15588572 | −0.0374677 | 0.13292819 | 0.11100106 | 0.1047354 | 0.29283322 |
| TNFRSF13C | | | | | | | | | |
| GHRL)- | −0.125190573 | 0.08696552 | 0.13475216 | 0.13367438 | −0.0895799 | 0.24246199 | 0.21681164 | 0.06498364 | 0.39355427 |
| PTGIR | | | | | | | | | |
| GNAS)- | −0.156793399 | −0.2182733 | −0.1055168 | −0.1790659 | −0.0646904 | −0.0958424 | −0.1678103 | −0.0292581 | −0.2673332 |
| PTGIR | | | | | | | | | |
| GNAI2)- | −0.375341373 | −0.2657147 | 0.06776067 | −0.1963266 | −0.0344089 | −0.020734 | −0.2220112 | 0.12191238 | −0.0472209 |
| CCR5 | | | | | | | | | |
| S100A4)- | 0.008451583 | −0.0731966 | 0.12509637 | 0.02827402 | −0.0183268 | 0.15410399 | 0.01707238 | −0.095701 | 0.00963752 |
| CCR5 | | | | | | | | | |
| CCL5)- | −0.200787442 | −0.0916751 | 0.46165541 | 0.14097545 | −0.3019459 | 0.24285526 | −0.1824417 | 0.25355551 | 0.31467293 |
| CCR5 | | | | | | | | | |
| CCL14)- | −0.069749441 | 0.02761761 | 0.25367422 | 0.24612857 | 0.00968187 | 0.16506721 | 0.12581719 | 0.18449963 | 0.50399416 |
| CCR5 | | | | | | | | | |
| CCL11)- | 0.08428984 | 0.17910733 | 0.04608225 | 0.0592277 | 0.31077048 | 0.05267693 | 0.13113949 | 0.25696305 | 0.31573921 |
| CCR5 | | | | | | | | | |
| CCL7)- | 0.026133944 | 0.27823566 | 0.2149196 | 0.27506168 | 0.17479264 | 0.26150519 | 0.35778676 | 0.08071749 | 0.38901436 |
| CCR5 | | | | | | | | | |
| CCL3L3)- | −0.107018192 | 0.04007965 | 0.26030591 | 0.05402539 | −0.2421122 | 0.20995083 | −0.0613436 | 0.21837469 | 0.33970223 |
| CCR5 | | | | | | | | | |
| CCL8)- | 0.096745735 | 0.15738461 | 0.12090998 | 0.24295884 | 0.23335836 | 0.21761741 | 0.16302553 | 0.16746558 | 0.49227894 |
| CCR5 | | | | | | | | | |
| MDK)- | −0.08022551 | 0.16256926 | 0.24664183 | 0.13989683 | 0.2382064 | 0.00587211 | −0.0661863 | −0.2537858 | −0.3488715 |
| GPC2 | | | | | | | | | |
| FGF19)- | 0.036638822 | −0.0336672 | 0.23310377 | −0.186872 | −0.0903434 | 0.01525512 | −0.1557522 | −0.0440856 | 0.09061402 |
| FGFR4 | | | | | | | | | |
| FGF21)- | −0.096374503 | 0.17847337 | 0.02421421 | −0.0510543 | −0.109013 | 0.12205068 | −0.0697927 | −0.1310963 | −0.0341857 |
| FGFR4 | | | | | | | | | |
| FGF14)- | −0.051256263 | 0.00478926 | 0.00605058 | −0.0236281 | −0.0735555 | −0.0386939 | −0.1541082 | −0.1413822 | −0.0836755 |
| FGFR4 | | | | | | | | | |
| FGF11)- | −0.025907117 | 0.11108317 | −0.1288004 | −0.0762245 | −0.0902482 | 0.05073486 | 0.10767636 | 0.09044513 | 0.07270954 |
| FGFR4 | | | | | | | | | |
| FGF17)- | −0.105336948 | 0.05573253 | 0.16878712 | 0.06969958 | −0.0093837 | 0.00912439 | −0.045466 | 0.01104701 | −0.0790633 |
| FGFR4 | | | | | | | | | |
| FGF12)- | 0.07611108 | −0.1224594 | 0.12860337 | 0.13808146 | −0.1037211 | −0.0225249 | −0.1217894 | −0.0576426 | −0.0975402 |
| FGFR4 | | | | | | | | | |
| L1CAM)- | 0.246453849 | 0.03364898 | −0.152929 | 0.03000078 | −0.0690991 | −0.038079 | −0.1090122 | 0.08871938 | 0.1969207 |
| ITGA5 | | | | | | | | | |
| PLAU)- | −0.069826985 | 0.2593115 | 0.32365426 | −0.0811304 | −0.0161958 | 0.31323851 | 0.20447908 | −0.1596928 | 0.4313432 |
| ITGA5 | | | | | | | | | |
| SPP1)- | 0.128261315 | −0.0680958 | 0.15410125 | 0.21579819 | 0.09228858 | −0.1350511 | 0.11971919 | −0.1416739 | 0.30645974 |
| ITGA5 | | | | | | | | | |
| CCN1)- | −0.348869467 | 0.29728772 | 0.31522795 | −0.1404687 | −0.1705526 | 0.30845227 | 0.17708878 | −0.1737419 | 0.29203576 |
| ITGA5 | | | | | | | | | |
| FGG)- | −0.024125549 | 0.14613614 | 0.04820464 | −0.0282942 | 0.02200286 | −0.0528817 | −0.0767243 | 0.0502449 | 0.11761828 |
| ITGA5 | | | | | | | | | |
| FGA)- | 0.066843436 | −0.0665679 | −0.1414912 | 0.18978909 | −0.0060883 | −0.0215608 | −0.0856296 | −0.0190937 | 0.05009725 |
| ITGA5 | | | | | | | | | |
| CCN2)- | −0.30972102 | 0.31670065 | 0.31876391 | −0.2347655 | −0.0843318 | 0.1323635 | 0.26745233 | −0.3235448 | 0.36581951 |
| ITGA5 | | | | | | | | | |
| FGB)- | 0.10774832 | −0.1245227 | −0.1452169 | 0.00817233 | −0.0772791 | 0.01882256 | −0.0301203 | 0.11806924 | 0.10287807 |
| ITGA5 | | | | | | | | | |
| ANGPT1)- | −0.034826499 | 0.00733698 | −0.1808881 | −0.0401028 | −0.1121106 | 0.16412521 | −0.041044 | −0.0792078 | 0.21164378 |
| ITGA5 | | | | | | | | | |
| COL18A1)- | −0.091393526 | 0.13787457 | 0.1772489 | −0.1145306 | −0.1248352 | 0.06433018 | 0.07574286 | −0.2663163 | 0.26042838 |
| ITGA5 | | | | | | | | | |
| ADAM15)- | −0.210523309 | 0.16831418 | 0.25974696 | −0.0056038 | −0.0433759 | 0.04164714 | 0.00373207 | −0.0212527 | 0.2404094 |
| ITGA5 | | | | | | | | | |
| ADAM17)- | 0.130424042 | 0.08356078 | 0.17778244 | −0.0534104 | −0.1024495 | 0.19229812 | 0.08235246 | −0.0415128 | 0.2536994 |
| ITGA5 | | | | | | | | | |
| FN1)- | −0.263870275 | 0.19368087 | 0.40405079 | −0.1774188 | −0.1297946 | 0.51814258 | 0.33663332 | −0.2323325 | 0.33611033 |
| ITGA5 | | | | | | | | | |
| GNAS)- | −0.191361016 | −0.2407561 | −0.0357029 | −0.2061924 | 0.07396793 | −0.2152547 | −0.0656176 | −0.0607667 | −0.1998655 |
| ADCY9 | | | | | | | | | |
| GNAI2)- | −0.034852761 | −0.2246502 | −0.0118908 | −0.2656132 | −0.0337206 | −0.0170785 | 0.02118578 | −0.0032061 | −0.1752796 |
| ADCY9 | | | | | | | | | |
| WNT8A)- | 0.145210529 | −0.2133399 | 0.18684163 | −0.309716 | 0.10550523 | 0.09404944 | 0.1320569 | 0.00498281 | −0.2694496 |
| LRP5 | | | | | | | | | |
| SOST)- | 0.162402571 | −0.276603 | 0.26733333 | −0.1914566 | −0.0679757 | 0.00475216 | 0.05224282 | −0.1057641 | −0.3474856 |
| LRP5 | | | | | | | | | |
| WNT7B)- | 0.072710654 | −0.4200929 | 0.21787914 | −0.3045849 | 0.07374305 | 0.11296656 | 0.04295109 | −0.1026246 | −0.0652509 |
| LRP5 | | | | | | | | | |
| DKK1)- | 0.100119243 | −0.0666487 | 0.09890058 | −0.020643 | 0.10471429 | 0.1904582 | 0.07879789 | −0.0040366 | −0.2554113 |
| LRP5 | | | | | | | | | |
| PSEN1)- | 0.016715226 | 0.06041963 | 0.0268697 | −0.0224966 | 0.03455132 | 0.03619719 | 0.02354062 | 0.15178258 | 0.11346909 |
| NCSTN | | | | | | | | | |
| GNAI2)- | −0.132410425 | −0.3541402 | −0.1823033 | −0.0757811 | −0.0904449 | −0.2801913 | −0.0716101 | −0.0797875 | −0.0274683 |
| LHCGR | | | | | | | | | |
| GNAS)- | −0.107561855 | −0.2433348 | −0.2182463 | −0.1739835 | −0.128589 | −0.1268494 | −0.1734408 | −0.0309311 | −0.1790169 |
| LHCGR | | | | | | | | | |
| GAS6)- | 0.050135 | −0.0501545 | 0.26109909 | −0.1784729 | 0.00245027 | −0.1989701 | 0.13508818 | 0.06109127 | 0.01532037 |
| MERTK | | | | | | | | | |
| PROS1)- | −0.052672249 | −0.11858 | 0.03514746 | 0.06717614 | −0.0216699 | 0.05476411 | −0.0573869 | −0.0237087 | −0.1060855 |
| MERTK | | | | | | | | | |
| CCK)- | 0.077960442 | 0.15280521 | 0.28966463 | 0.13959948 | −0.1325626 | 0.24790728 | 0.07006896 | 0.16316255 | 0.21473421 |
| CCKAR | | | | | | | | | |
| YARS1)- | −0.076537503 | −0.2999132 | 0.09405867 | 0.02603095 | −0.1522701 | 0.16795714 | 0.12291265 | 0.07694189 | 0.16831492 |
| CXCR1 | | | | | | | | | |
| GNAI2)- | −0.224944442 | −0.2829295 | −0.0067241 | −0.2202159 | −0.0541785 | −0.0810235 | 0.03485145 | −0.1152908 | −0.0709282 |
| CXCR1 | | | | | | | | | |
| CXCL3)- | −0.139186396 | −0.0668708 | 0.04578464 | −0.0378625 | −0.1120624 | −0.0557464 | 0.03409265 | −0.0943123 | −0.1549753 |
| CXCR1 | | | | | | | | | |
| CXCL8)- | −0.06763951 | −0.0058124 | 0.03160724 | 0.09088432 | 0.00982017 | 0.14975618 | 0.14830698 | 0.01720587 | 0.15063852 |
| CXCR1 | | | | | | | | | |
| CXCL2)- | −0.095696226 | 0.01676091 | 0.13093897 | 0.17112163 | 0.04849052 | 0.15208076 | 0.06166799 | 0.00094775 | 0.15211536 |
| CXCR1 | | | | | | | | | |
| PPBP)- | −0.069867847 | 0.18596171 | 0.12100598 | 0.07630128 | −0.0379146 | 0.16247145 | 0.14949129 | 0.05580417 | 0.39319161 |
| CXCR1 | | | | | | | | | |
| CXCL5)- | −0.102329275 | 0.15599825 | 0.11453662 | 0.00268922 | −0.0205727 | 0.00733463 | 0.02775136 | −0.0672298 | −0.0245245 |
| CXCR1 | | | | | | | | | |
| CXCL1)- | −0.169811847 | −0.1452537 | 0.02105876 | 0.11064954 | −0.1171896 | 0.19614306 | 0.09850496 | 0.07389178 | 0.03398916 |
| CXCR1 | | | | | | | | | |
| IL17F)- | −0.092044125 | 0.1509033 | 0.12737709 | 0.09282144 | −0.0794593 | 0.02642581 | 0.18410014 | −0.0235308 | 0.31286132 |
| IL17RC | | | | | | | | | |
| FN1)- | −0.112938085 | −0.0223189 | −0.0572345 | −0.1477823 | 0.00421992 | −0.0764088 | 0.02599063 | −0.0762643 | 0.09901866 |
| IL17RC | | | | | | | | | |
| CCL5)- | −0.257995227 | −0.2126807 | 0.33781709 | 0.16585939 | −0.2958856 | 0.23984015 | −0.1873981 | 0.12109129 | 0.30751182 |
| CCR1 | | | | | | | | | |
| CCL14)- | −0.202147112 | −0.1187565 | 0.15893384 | −0.0045358 | −0.057805 | 0.09318243 | 0.23058867 | −0.0987693 | 0.16786232 |
| CCR1 | | | | | | | | | |
| CCL15)- | −0.041322922 | 0.08856157 | −0.0604049 | 0.04008895 | −0.1218057 | 0.03855108 | 0.10340313 | −0.1921009 | 0.00263351 |
| CCR1 | | | | | | | | | |
| CCL23)- | −0.130749737 | 0.10122532 | 0.13373635 | 0.13980646 | −0.2211823 | 0.1646747 | 0.16087091 | −0.1761711 | 0.25360307 |
| CCR1 | | | | | | | | | |
| CCL7)- | −0.187184138 | 0.03586854 | 0.21411 | 0.12153238 | −0.019915 | 0.15608118 | 0.07506256 | −0.1191915 | 0.19791432 |
| CCR1 | | | | | | | | | |
| CCL8)- | −0.113634475 | 0.06702348 | 0.29318359 | 0.10250558 | −0.1355989 | 0.10455568 | 0.10614948 | −0.1441584 | 0.31743823 |
| CCR1 | | | | | | | | | |
| CCL18)- | −0.098717118 | −0.1390524 | 0.20804631 | −0.0166659 | −0.0603087 | 0.07729406 | 0.002743 | −0.0549453 | 0.09641875 |
| CCR1 | | | | | | | | | |
| PLAU)- | −0.08772341 | 0.12567926 | 0.21117896 | −0.2594492 | −0.2141181 | 0.34448398 | 0.10429894 | −0.2924506 | 0.30704064 |
| ITGB5 | | | | | | | | | |
| EDIL3)- | −0.433550209 | 0.24672863 | 0.18808597 | −0.2773064 | −0.0915733 | 0.59059755 | 0.16296741 | −0.504096 | 0.38043445 |
| ITGB5 | | | | | | | | | |
| SORBS1)- | −0.141549054 | 0.02331069 | 0.08745697 | −0.263683 | −0.1537521 | 0.35867036 | −0.008744 | −0.1724622 | −0.0163017 |
| ITGB5 | | | | | | | | | |
| SPP1)- | 0.234897422 | 0.13286383 | −0.0100379 | 0.01050461 | 0.18931903 | −0.1022231 | 0.18590699 | −0.1707222 | 0.08503246 |
| ITGB5 | | | | | | | | | |
| TLN1)- | −0.062772807 | 0.10046109 | 0.10286628 | −0.1774885 | −0.1006774 | 0.27327416 | 0.14611833 | −0.2080198 | 0.24203184 |
| ITGB5 | | | | | | | | | |
| COL4A2)- | −0.111878125 | 0.1644848 | 0.352084 | 0.060043 | 0.03244998 | 0.39869611 | 0.2986364 | −0.2236622 | 0.21521755 |
| ITGB5 | | | | | | | | | |
| CCN1)- | −0.321373488 | 0.19316729 | 0.26559442 | −0.3844878 | −0.2401321 | 0.50667325 | 0.18876016 | −0.4435279 | 0.29880586 |
| ITGB5 | | | | | | | | | |
| ADAM9)- | −0.091864743 | 0.00703041 | 0.15256896 | −0.0081348 | 0.08715992 | 0.01876877 | 0.08727709 | 0.05459552 | 0.33444356 |
| ITGB5 | | | | | | | | | |
| COL18A1)- | −0.018945225 | 0.17312343 | 0.29598664 | 0.04231364 | −0.1456941 | −0.0853573 | 0.19172464 | −0.2774136 | 0.21967599 |
| ITGB5 | | | | | | | | | |
| ITGB3BP)- | −0.033991757 | −0.1129744 | 0.02456046 | −0.1848735 | −0.1462347 | −0.0679121 | −0.1954543 | −0.0291358 | −0.3591276 |
| ITGB5 | | | | | | | | | |
| LTBP3)- | −0.115101475 | 0.23128874 | 0.21520836 | 0.07647611 | 0.0753894 | 0.33611198 | 0.1741926 | −0.1910844 | 0.27102414 |
| ITGB5 | | | | | | | | | |
| LTBP1)- | −0.049364832 | 0.16674063 | 0.31320487 | −0.2620727 | −0.1895468 | 0.46246171 | 0.29348663 | −0.3229569 | 0.31762971 |
| ITGB5 | | | | | | | | | |
| MST1)- | −0.014201288 | −0.0173094 | −0.0079485 | −0.288074 | 0.03457511 | 0.16045768 | 0.08986503 | 0.09842577 | −0.1880635 |
| MST1R | | | | | | | | | |
| SHH)- | −0.120330536 | 0.01659976 | 0.2891763 | 0.13066467 | −0.0305154 | 0.11256813 | 0.05922078 | 0.16694782 | 0.10528983 |
| HHIP | | | | | | | | | |
| COL4A5)- | −0.136033814 | −0.014862 | −0.1627657 | −0.1026252 | 0.01592417 | 0.10638273 | 0.04665258 | 0.03447917 | 0.13365966 |
| ITGA2 | | | | | | | | | |
| LAMA2)- | −0.015976491 | −0.0447377 | 0.01344914 | −0.0515988 | −0.0280543 | −0.0175888 | 0.0481026 | 0.04879521 | −0.2802875 |
| ITGA2 | | | | | | | | | |
| LAMA1)- | −0.235434121 | −0.0112682 | −0.180983 | −0.0635891 | 0.01558998 | 0.0209923 | −0.0812336 | −0.0295286 | −0.2413866 |
| ITGA2 | | | | | | | | | |
| LAMC3)- | −0.108202726 | 0.04029355 | 0.00448048 | −0.2786812 | −0.1549733 | −0.0547884 | 0.02257342 | 0.01788696 | −0.1532918 |
| ITGA2 | | | | | | | | | |
| COL4A4)- | −0.126574241 | 0.16841049 | −0.1585895 | −0.3435373 | 0.1151921 | 0.12430218 | −0.1249601 | −0.0935951 | −0.216054 |
| ITGA2 | | | | | | | | | |
| COL11A1)- | −0.048631159 | −0.0824666 | −0.0425281 | 0.18255301 | −0.0709136 | −0.0700658 | −0.077762 | −0.0230544 | −0.1747433 |
| ITGA2 | | | | | | | | | |
| COL6A2)- | 0.14611297 | −0.2341003 | 0.20045112 | 0.40659988 | −0.1598705 | −0.2028761 | 0.16414793 | 0.13140735 | −0.1563178 |
| ITGA2 | | | | | | | | | |
| COL4A3)- | −0.01538162 | −0.0385337 | 0.05854652 | −0.2117171 | −0.0661429 | −0.1352192 | −0.0811926 | −0.120197 | −0.0191696 |
| ITGA2 | | | | | | | | | |
| MMP1)- | 0.069322943 | 0.07890982 | 0.02432919 | −0.1202428 | 0.03813339 | −0.002408 | −0.0581268 | −0.0832275 | −0.0999108 |
| ITGA2 | | | | | | | | | |
| HSPG2)- | 0.105018624 | −0.086877 | 0.05648165 | 0.37371101 | −0.0593713 | −0.0586946 | 0.0444145 | 0.04522419 | −0.0127455 |
| ITGA2 | | | | | | | | | |
| LAMB3)- | 0.371771061 | −0.0316573 | 0.36771585 | −0.0215335 | 0.33955328 | 0.44983976 | −0.2398711 | −0.342852 | 0.38363676 |
| ITGA2 | | | | | | | | | |
| COL1A2)- | −0.014629459 | −0.165346 | 0.08771293 | 0.34159259 | −0.043155 | −0.0056948 | 0.02518638 | 0.0007675 | −0.0748795 |
| ITGA2 | | | | | | | | | |
| FN1)- | −0.052330929 | −0.0660037 | −0.0001025 | 0.35598116 | −0.0824731 | −0.0660148 | 0.01789528 | −0.0062338 | 0.02253241 |
| ITGA2 | | | | | | | | | |
| COL7A1)- | −0.053629845 | −0.0473292 | 0.12980811 | 0.02194158 | 0.06356614 | 0.10923035 | 0.11697417 | 0.06118662 | 0.18906216 |
| ITGA2 | | | | | | | | | |
| COL3A1)- | 0.044503553 | −0.1448817 | 0.14025046 | 0.34680681 | −0.1031594 | −0.0847625 | 0.08176932 | 0.03140324 | −0.0508327 |
| ITGA2 | | | | | | | | | |
| F2)- F2RL2 | 0.219298895 | 0.35599015 | 0.12062237 | 0.16574474 | −0.0428513 | 0.25358887 | −0.0788781 | 0.25485851 | 0.41704516 |
| F2)- F2RL1 | 0.083622941 | −0.100298 | 0.20561078 | −0.3401228 | 0.13574787 | 0.16666789 | 0.2025056 | 0.18052154 | −0.3522314 |
| NUCB2)- | 0.014722603 | 0.0900275 | 0.0513141 | −0.0614385 | 0.08769141 | 0.07006214 | −0.0560818 | 0.22263106 | 0.18753598 |
| ERAPI | | | | | | | | | |
| TNF)- | −0.041673995 | 0.02161705 | −0.0700731 | −0.0869657 | 0.13166607 | 0.04099134 | 0.05802408 | 0.00418017 | −0.1215727 |
| TNFRSF21 | | | | | | | | | |
| TNFSF11)- | 0.081425507 | 0.0299317 | 0.19095715 | 0.19694127 | 0.0287313 | 0.18181978 | 0.14586507 | 0.16968802 | 0.13375458 |
| TNFRSF11B | | | | | | | | | |
| FN1)- | −0.000222664 | −0.054855 | −0.0643113 | 0.12021267 | −0.069635 | 0.02944167 | −0.1061397 | −0.0234266 | 0.11313138 |
| TNFRSF11B | | | | | | | | | |
| INSL3)- | 0.096891252 | 0.10634565 | 0.13476512 | 0.17260251 | −0.0629591 | 0.19362569 | 0.13819429 | 0.07247889 | 0.39340252 |
| RXFP2 | | | | | | | | | |
| FAT4)- | −0.040228337 | 0.19880826 | 0.04618305 | −0.0659026 | −0.035973 | 0.00930367 | 0.11303288 | −0.028534 | 0.46859388 |
| DCHS1 | | | | | | | | | |
| ADM)- | 0.052187358 | 0.05774212 | 0.27502226 | 0.0666234 | 0.01197471 | 0.19244552 | 0.02259795 | 0.08927081 | 0.12084622 |
| GPR182 | | | | | | | | | |
| B2M)- | −0.149278829 | −0.1824499 | 0.23254843 | −0.1384604 | 0.07368143 | −0.1572708 | 0.10060909 | 0.01059602 | −0.0639998 |
| CD3D | | | | | | | | | |
| HLA-C)- | 0.005246853 | −0.198779 | 0.06444537 | 0.06037917 | 0.10089634 | 0.04198275 | 0.20407482 | −0.1340017 | 0.00870303 |
| CD3D | | | | | | | | | |
| HLA-B)- | −0.065838389 | −0.2198654 | 0.11936333 | 0.00768655 | 0.079526 | 0.0318467 | 0.16745516 | −0.0535367 | −0.0307462 |
| CD3D | | | | | | | | | |
| GAS6)- | −0.209470129 | 0.09582719 | 0.23456278 | −0.2692947 | 0.00168891 | 0.39288262 | 0.1529928 | −0.101653 | 0.14182351 |
| AXL | | | | | | | | | |
| PROS1)- | 0.064693127 | −0.0444775 | 0.0336991 | −0.1602975 | −0.1208704 | 0.01974571 | −0.0232827 | 0.04418535 | −0.1177034 |
| AXL | | | | | | | | | |
| SPP1)- | −0.183816065 | −0.1932314 | 0.01502519 | 0.1989428 | −0.1800308 | 0.1554567 | −0.0950478 | 0.09162548 | 0.26202193 |
| PTGER4 | | | | | | | | | |
| LRPAP1)- | −0.008132885 | 0.07591832 | 0.21598695 | 0.02413557 | 0.02792451 | 0.15732524 | 0.09290817 | −0.0333681 | 0.35869322 |
| LRP8 | | | | | | | | | |
| SORBS1)- | 0.051834977 | 0.03139337 | 0.14696654 | 0.0761835 | −0.1182865 | −0.1439167 | 0.03024701 | −0.0003252 | −0.0750961 |
| INSR | | | | | | | | | |
| INS)- INSR | 0.181541662 | −0.2381957 | 0.16446281 | −0.0289524 | −0.239634 | −0.0358095 | 0.11247968 | 0.0804362 | −0.0863947 |
| AHSG)- | 0.000681365 | −0.076138 | 0.05863281 | −0.1645833 | −0.1106058 | 0.02055937 | −0.0720598 | 0.11321963 | −0.0922708 |
| INSR | | | | | | | | | |
| ARF1)- | 0.15981425 | −0.0644685 | 0.23023471 | −0.008744 | 0.1510451 | 0.09200619 | 0.20119201 | −0.1186237 | 0.06651913 |
| INSR | | | | | | | | | |
| GIP)- INSR | −0.138248726 | −0.1266892 | −0.0492843 | −0.0365532 | −0.1148057 | 0.16387841 | −0.1892133 | 0.09757863 | −0.0243456 |
| IGF2)- | 0.002311742 | −0.0512887 | 0.10611938 | −0.1131689 | 0.0030658 | −0.0599449 | 0.15118527 | 0.1496975 | −0.1888768 |
| INSR | | | | | | | | | |
| HRAS)- | −0.006792083 | 0.0224213 | 0.04172364 | −0.1074911 | 0.07172696 | 0.07714435 | 0.04157121 | 0.07390138 | −0.1138253 |
| INSR | | | | | | | | | |
| CALM1)- | 0.073264925 | 0.02368878 | 0.1128418 | 0.07149218 | −0.2150633 | −0.042392 | 0.05250831 | 0.24525035 | 0.00271841 |
| INSR | | | | | | | | | |
| CD86)- | −0.180332415 | −0.0185893 | 0.29169058 | 0.09444897 | −0.065052 | 0.27166283 | −0.0220749 | 0.22840049 | 0.46315703 |
| CTLA4 | | | | | | | | | |
| F2)- GP9 | −0.042337207 | 0.25456417 | 0.15542719 | 0.19656387 | 0.16353268 | 0.14257773 | −0.0142653 | 0.16463716 | 0.53792671 |
| DKK1)- | −0.189418343 | 0.07736041 | 0.00500494 | −0.0151542 | −0.1079107 | −0.0077238 | 0.10511636 | 0.14879456 | 0.03575619 |
| KREMEN2 | | | | | | | | | |
| ACE2)- | −0.027676217 | 0.15221127 | 0.08577506 | 0.15815967 | 0.12239123 | 0.33414108 | 0.19772394 | 0.26426959 | 0.32322463 |
| SLC6A19 | | | | | | | | | |
| GHRH)- | 0.108902607 | 0.20740149 | 0.08783246 | 0.26450936 | −0.0369055 | 0.20697892 | 0.21219803 | −0.0363873 | 0.51356426 |
| ADRB2 | | | | | | | | | |
| PTHLH)- | 0.122187593 | 0.3483359 | 0.23013787 | −0.1097216 | 0.0667848 | 0.05811164 | 0.07436531 | 0.21707767 | 0.46323905 |
| ADRB2 | | | | | | | | | |
| POMC)- | 0.074153271 | 0.30666896 | 0.11511741 | 0.07375369 | 0.11268764 | 0.06133961 | 0.12017793 | 0.13938157 | 0.45278879 |
| ADRB2 | | | | | | | | | |
| HSPA8)- | −0.069314388 | −0.0694339 | 0.13726583 | 0.07335975 | 0.13373293 | −0.1045789 | 0.04441378 | 0.00490478 | −0.141771 |
| ADRB2 | | | | | | | | | |
| ADCYAP1)- | 0.112249722 | 0.27611369 | 0.1842266 | 0.08552029 | 0.0550912 | 0.13520539 | 0.18232819 | 0.0708545 | 0.5232722 |
| ADRB2 | | | | | | | | | |
| CALCA)- | 0.048434844 | 0.00741381 | 0.11675414 | 0.24844445 | −0.0602678 | 0.25619101 | 0.01523285 | 0.2580512 | 0.39247105 |
| ADRB2 | | | | | | | | | |
| ADM)- | 0.066736323 | 0.21668973 | 0.31255578 | 0.16569841 | −0.04938 | 0.03888807 | −0.0964956 | 0.12493078 | 0.20805212 |
| ADRB2 | | | | | | | | | |
| GCG)- | −0.156289166 | −0.0557953 | 0.17199571 | 0.23158342 | −0.1133882 | 0.31550656 | 0.13446891 | 0.17447993 | 0.4320498 |
| ADRB2 | | | | | | | | | |
| ADM2)- | −0.03926951 | 0.20172723 | 0.05732456 | 0.0171422 | 0.08301071 | 0.09905303 | 0.04186733 | 0.01079988 | −0.1122841 |
| ADRB2 | | | | | | | | | |
| NPS)- | −0.021438904 | 0.21284735 | 0.21310872 | 0.10032929 | −0.0242227 | 0.19820021 | 0.09394935 | 0.23297066 | 0.65075262 |
| ADRB2 | | | | | | | | | |
| FSHB)- | −0.018379703 | 0.24398497 | 0.13774542 | 0.13824861 | 0.00195943 | 0.25352175 | 0.12751232 | 0.06118216 | 0.56177142 |
| ADRB2 | | | | | | | | | |
| CALCB)- | 0.137017259 | 0.14792925 | 0.17827636 | 0.19655556 | 0.0778525 | 0.17177664 | 0.14236592 | 0.14206417 | 0.52751666 |
| ADRB2 | | | | | | | | | |
| ACTR2)- | 0.012075076 | 0.14881802 | 0.02129084 | 0.08308263 | 0.21318363 | −0.1027261 | 0.13865875 | −0.0637707 | −0.1075705 |
| ADRB2 | | | | | | | | | |
| GIP)- | 0.009951925 | 0.16727046 | 0.43086642 | 0.26867693 | −0.097382 | 0.2825633 | 0.06988036 | −0.0153428 | 0.44653761 |
| ADRB2 | | | | | | | | | |
| INSL3)- | 0.083553002 | 0.15741032 | −0.0618484 | −0.0062184 | 0.24247599 | 0.17437367 | 0.10880468 | 0.08032253 | 0.52497847 |
| ADRB2 | | | | | | | | | |
| PENK)- | −0.162548307 | 0.12766768 | 0.21614998 | 0.14057847 | −0.022392 | 0.15813389 | 0.08581245 | 0.00464447 | 0.44676382 |
| MRGPRX1 | | | | | | | | | |
| NMS)- | −0.051599782 | 0.15584063 | 0.16900329 | 0.30209435 | 0.05799605 | 0.17691251 | 0.13216706 | 0.15788472 | 0.47398824 |
| NMUR1 | | | | | | | | | |
| CD6)- | −0.021528329 | 0.08588549 | 0.15927429 | 0.18737732 | −0.0223544 | 0.29896368 | 0.09033445 | 0.13392322 | 0.147293 |
| ALCAM | | | | | | | | | |
| S100A9)- | −0.031462676 | −0.1586773 | 0.21918923 | 0.25034969 | −0.0818212 | 0.15011954 | −0.1833058 | 0.08184678 | 0.17303845 |
| ALCAM | | | | | | | | | |
| TSLP)- | −0.006404819 | 0.09386523 | 0.07372889 | −0.0218631 | 0.10195448 | 0.0157991 | 0.02522156 | 0.15215029 | 0.33241353 |
| IL7R | | | | | | | | | |
| LAMA3)- | −0.191545459 | 0.29012584 | 0.08489459 | 0.32847066 | 0.04784036 | −0.1763853 | −0.1014876 | −0.0362236 | −0.1399494 |
| SDC2 | | | | | | | | | |
| LAMA1)- | −0.091440502 | 0.16326401 | 0.03089108 | −0.1261171 | −0.0474058 | 0.06061268 | −0.0717638 | −0.1656055 | 0.35419752 |
| SDC2 | | | | | | | | | |
| HRAS)- | −0.028025416 | 0.07635258 | 0.01891366 | 0.09238555 | 0.05890774 | −0.0316093 | −0.1694826 | −0.0205696 | 0.07929426 |
| SDC2 | | | | | | | | | |
| SERPINC1)- | 0.110111839 | −0.0491661 | −0.1350649 | 0.07887983 | 0.0005069 | −0.0665958 | −0.0619983 | −0.1333277 | 0.31408522 |
| SDC2 | | | | | | | | | |
| FN1)- SDC2 | −0.347904675 | 0.33460466 | 0.46321161 | −0.1382359 | −0.0159948 | 0.35438638 | 0.38853484 | −0.349718 | 0.46734393 |
| GNAI2)- | −0.164979556 | −0.3663771 | −0.0150463 | −0.1140078 | −0.0320351 | 0.06541504 | −0.1525545 | 0.07574115 | −0.0448216 |
| P2RY12 | | | | | | | | | |
| PLAU)- | 0.019210655 | 0.12987565 | 0.09388059 | −0.1085998 | −0.0667061 | 0.43931419 | 0.10590275 | 0.07066455 | 0.23336214 |
| MRC2 | | | | | | | | | |
| CALM1)- | −0.01264176 | −0.013264 | 0.01761077 | −0.0131582 | −0.0249895 | 0.09822487 | −0.0872629 | −0.0295982 | −0.1881885 |
| GP6 | | | | | | | | | |
| XCL1)- | 0.058109939 | −0.2040338 | 0.12882224 | −0.1091666 | −0.0043454 | 0.01014044 | 0.14478599 | 0.12656261 | −0.032684 |
| XCR1 | | | | | | | | | |
| CXCL2)- | 0.021339342 | 0.14649081 | 0.0726071 | 0.17970728 | 0.03887604 | 0.08185311 | 0.21787766 | 0.13205864 | −0.2375309 |
| XCR1 | | | | | | | | | |
| CD200)- | −0.170574697 | 0.22034845 | 0.22998114 | 0.16869168 | 0.21402518 | 0.04530364 | 0.25845809 | 0.01303133 | 0.41077761 |
| CD200R1 | | | | | | | | | |
| CALM1)- | 0.038154128 | −0.0896841 | 0.17716029 | −0.0658266 | −0.0303592 | 0.09678983 | −0.0306149 | −6.15E−05 | 0.05588283 |
| AQP1 | | | | | | | | | |
| RARRES2)- | −0.069843344 | 0.0794498 | 0.0163527 | −0.1722606 | −0.0060387 | −0.0491621 | −0.0065321 | −0.0176428 | 0.30088319 |
| CMKLR1 | | | | | | | | | |
| C4B)- CD46 | 0.175209785 | 0.0595793 | 0.07961977 | −0.0322733 | −0.0762145 | −0.2117144 | 0.19961333 | 0.06402636 | −0.3629791 |
| IL24)- | 0.084760934 | 0.1691055 | 0.09886042 | 0.23861145 | 0.05304674 | 0.23002923 | 0.1435813 | 0.14634354 | 0.27924669 |
| IL20RA | | | | | | | | | |
| EDN3)- | −0.163504366 | 0.18072257 | 0.08638596 | −0.0670393 | −0.3003459 | 0.18370583 | −0.0102039 | −0.0622269 | 0.19411668 |
| EDNRA | | | | | | | | | |
| EDN1)- | −0.090559411 | 0.18300083 | 0.0701369 | 0.00523839 | −0.0051524 | −0.1136377 | 0.02109456 | 0.02383234 | −0.0153585 |
| EDNRA | | | | | | | | | |
| SEMA4A)- | −0.009466865 | −0.0609388 | 0.0365425 | −0.0633813 | −0.0713244 | −0.0551955 | −0.1002613 | 0.14482259 | −0.0703509 |
| PLXND1 | | | | | | | | | |
| SEMA4D)- | −0.221553816 | −0.0186182 | −0.1100636 | −0.1676226 | 0.03232563 | 0.18314719 | −0.0375246 | 0.09023548 | −0.1888134 |
| MET | | | | | | | | | |
| SEMA5A)- | 0.009777036 | 0.15345558 | 0.14832449 | −0.1998081 | −0.1034795 | −0.0882763 | 0.10211982 | −0.0532646 | 0.16001336 |
| MET | | | | | | | | | |
| TNF)- ICOS | 0.091239704 | 0.23841089 | 0.13853097 | 0.11526072 | 0.05799216 | 0.20942474 | 0.18177185 | 0.23825768 | 0.58006591 |
| ICOSLG)- | −0.093573148 | −0.060903 | 0.30886185 | 0.18984794 | −0.0997306 | 0.15742177 | 0.00017001 | 0.26022305 | 0.20736903 |
| ICOS | | | | | | | | | |
| CXCL2)- | −0.068851907 | 0.13954156 | −0.0150141 | 0.38297729 | −0.0283897 | 0.18863549 | 0.15508239 | 0.07068457 | 0.01593851 |
| CXCR2 | | | | | | | | | |
| CXCL1)- | −0.008471187 | −0.0811262 | −0.055105 | 0.17774367 | −0.0565608 | −0.0369303 | 0.12200588 | −0.0552016 | −0.0515854 |
| CXCR2 | | | | | | | | | |
| CD55)- | 0.074635567 | 0.17826997 | 0.00067121 | 0.22658074 | −0.0580451 | 0.15853014 | 0.12776418 | −0.3791682 | −0.3828799 |
| ADGRE2 | | | | | | | | | |
| IL17F)- | 0.095497196 | 0.16534576 | 0.10596902 | 0.02916721 | 0.12605188 | 0.05344406 | 0.06814943 | −0.0283075 | 0.19676131 |
| IL17RA | | | | | | | | | |
| SEMA4B)- | −0.099309385 | 0.29681479 | 0.40835551 | 0.30917567 | 0.33967151 | −0.1000004 | −0.1666967 | 0.22950901 | −0.2845225 |
| DCBLD2 | | | | | | | | | |
| GHRH)- | 0.121743095 | 0.05721805 | 0.2070716 | 0.17806636 | 0.02470323 | 0.09548678 | 0.22972987 | 0.20027277 | 0.45916368 |
| PTH1R | | | | | | | | | |
| PTHLH)- | 0.036805807 | 0.04282929 | 0.0550228 | 0.12233686 | −0.103257 | 0.13638769 | 0.21094882 | 0.10134142 | 0.46587252 |
| PTH1R | | | | | | | | | |
| POMC)- | 0.093940587 | 0.08574745 | 0.1988531 | 0.20672367 | 0.00165495 | 0.3094817 | 0.13819673 | 0.11942107 | 0.48019556 |
| PTH1R | | | | | | | | | |
| ADCYAP1)- | 0.010349425 | 0.11095854 | 0.15897869 | 0.11342059 | 0.05562143 | 0.1312545 | 0.22377455 | 0.17535268 | 0.53358643 |
| PTH1R | | | | | | | | | |
| CALCA)- | −0.108693806 | 0.12688167 | 0.15212873 | 0.11671216 | 0.09286564 | 0.30537561 | 0.21685501 | 0.15627971 | 0.48383964 |
| PTH1R | | | | | | | | | |
| ADM)- | 0.123092862 | 0.15769642 | 0.12344005 | 0.12125253 | 0.03621358 | 0.17031202 | −0.0258809 | 0.07746149 | 0.2084798 |
| PTH1R | | | | | | | | | |
| GCG)- | −0.113784648 | −0.0223005 | 0.11039277 | 0.17739857 | 0.15080997 | 0.0761225 | 0.25048799 | 0.04195074 | 0.35161626 |
| PTH1R | | | | | | | | | |
| ADM2)- | 0.074569517 | 0.05499307 | 0.10753363 | −0.037724 | 0.01345547 | 0.01120395 | −0.0445524 | 0.08493131 | −0.0071505 |
| PTH1R | | | | | | | | | |
| NPS)- | −0.04862643 | 0.26615736 | 0.20628519 | 0.0174901 | −0.0064585 | 0.10732892 | 0.2872253 | 0.15984384 | 0.54859824 |
| PTH1R | | | | | | | | | |
| FSHB)- | −0.057290984 | 0.10460895 | 0.25553917 | 0.27909629 | 0.05766781 | 0.26548678 | 0.17548684 | 0.3190607 | 0.51485241 |
| PTH1R | | | | | | | | | |
| CALCB)- | 0.097677856 | 0.2503143 | 0.10034096 | 0.09038889 | 0.08159458 | 0.22521795 | 0.11446416 | 0.24970922 | 0.4109762 |
| PTH1R | | | | | | | | | |
| GIP)- | 0.016216493 | 0.27021627 | 0.32832147 | 0.18439216 | 0.04503195 | 0.26509156 | 0.10237783 | 0.18743545 | 0.52787034 |
| PTH1R | | | | | | | | | |
| INSL3)- | 0.168691167 | 0.111402 | 0.14006892 | −0.0851083 | 0.03921709 | −0.0333158 | 0.08454055 | 0.17577699 | 0.45905371 |
| PTH1R | | | | | | | | | |
| PDX1)- | 0.001980381 | 0.02391303 | 0.1468399 | 0.30822786 | 0.12137372 | 0.06162228 | 0.13120324 | 0.07739031 | 0.16427461 |
| SLC2A2 | | | | | | | | | |
| CD86)- | 0.134646829 | 0.05249755 | 0.08121255 | 0.1573837 | 0.05105116 | 0.00408353 | 0.15400226 | 0.20312935 | 0.39877027 |
| CD28 | | | | | | | | | |
| GHRH)- | −0.166019915 | −0.0579201 | 0.06612503 | 0.04214928 | 0.00965117 | 0.28710215 | −0.0897534 | 0.13766664 | 0.2238179 |
| VIPR1 | | | | | | | | | |
| PTHLH)- | −0.111503955 | 0.0251035 | 0.03759984 | 0.12889265 | −0.0532832 | 0.07037585 | 0.03732292 | 0.21192789 | 0.00829005 |
| VIPR1 | | | | | | | | | |
| POMC)- | 0.122849595 | 0.12080956 | 0.02215579 | 0.05174268 | 0.01109458 | 0.08071104 | −0.0414305 | 0.20370308 | 0.09433349 |
| VIPR1 | | | | | | | | | |
| ADCYAP1)- | 0.069152998 | −0.0427321 | 0.00263859 | 0.0655219 | −0.0810345 | 0.19040021 | −0.1174091 | 0.19004096 | 0.20835366 |
| VIPR1 | | | | | | | | | |
| CALCA)- | −0.010935256 | 0.09463493 | 0.10148652 | 0.14834415 | −0.047107 | 0.17140739 | 0.03221708 | 0.25991876 | 0.13212195 |
| VIPR1 | | | | | | | | | |
| ADM)- | 0.012643203 | −0.0865257 | −0.0216822 | 0.04963003 | −0.031681 | 0.20824107 | 0.06014316 | 0.00518589 | 0.00371147 |
| VIPR1 | | | | | | | | | |
| GCG)- | −0.117943464 | −0.1479148 | 0.13355353 | 0.09094078 | −0.2377111 | 0.21969468 | 0.09153833 | 0.04103747 | 0.08212413 |
| VIPR1 | | | | | | | | | |
| ADM2)- | −0.0779681 | 0.00569245 | 0.06715232 | −0.0682849 | 0.13425176 | 0.37120612 | 0.02069259 | −0.0359087 | 0.32709774 |
| VIPR1 | | | | | | | | | |
| GNAS)- | −0.178823986 | 0.01247363 | −0.0885698 | 0.0596273 | −0.05676 | −0.1688934 | 0.03910184 | 0.01068527 | 0.06847319 |
| VIPR1 | | | | | | | | | |
| NPS)- | 0.023597053 | 0.16774367 | 0.07825903 | 0.09685221 | −0.1499754 | 0.14733159 | −0.085965 | 0.15129626 | 0.14149877 |
| VIPR1 | | | | | | | | | |
| FSHB)- | 0.09651443 | 0.06538858 | 0.17293362 | 0.05547872 | −0.0145696 | 0.28555603 | −0.0728797 | 0.16279898 | 0.18946815 |
| VIPR1 | | | | | | | | | |
| PTMA)- | 0.049192673 | 0.05208063 | −0.2440312 | 0.0827947 | −0.2441748 | 0.07453613 | −0.0228585 | −0.1232131 | −0.0730342 |
| VIPR1 | | | | | | | | | |
| CALM1)- | −0.124452793 | −0.1598765 | −0.004712 | 0.33435428 | −0.1235484 | −0.0001436 | −0.1378385 | 0.0464577 | −0.1070585 |
| VIPR1 | | | | | | | | | |
| CALCB)- | −0.142420852 | 0.04018028 | 0.11792026 | 0.17040506 | 0.00578547 | 0.40427649 | −0.0784838 | 0.2788887 | 0.29217851 |
| VIPR1 | | | | | | | | | |
| GIP)- | 0.052857065 | 0.06036524 | 0.12732705 | 0.06405553 | −0.1334649 | 0.17707715 | −0.0057579 | 0.12853692 | 0.2754662 |
| VIPR1 | | | | | | | | | |
| INSL3)- | −0.054408353 | 0.09458497 | 0.19417852 | −0.1102006 | 0.03005945 | 0.13959538 | −0.0729137 | 0.03496978 | 0.13816139 |
| VIPR1 | | | | | | | | | |
| IL24)- | 0.048436211 | 0.25803054 | 0.22311117 | 0.20372125 | 0.1146436 | 0.04252086 | 0.27560651 | 0.13868165 | 0.41126568 |
| IL20RB | | | | | | | | | |
| TNFSF18)- | −0.130445299 | 0.13369808 | 0.13220907 | 0.19324688 | 0.03076114 | 0.19623705 | 0.19153366 | 0.02858244 | 0.4393972 |
| TNFRSF18 | | | | | | | | | |
| F11)- | 0.054058846 | 0.08274316 | 0.19400111 | 0.04686188 | −0.0485212 | 0.28124359 | 0.19792648 | 0.24654191 | 0.4683068 |
| GP1BA | | | | | | | | | |
| APOD)- | −0.094828922 | −0.2015347 | −0.1103089 | 0.18945454 | 0.18209924 | 0.01667724 | −0.1385905 | 0.12713244 | 0.08491834 |
| LEPR | | | | | | | | | |
| DKK1)- | −0.105151504 | 0.10867941 | 0.16240766 | −0.0251764 | −0.1111677 | −0.0346733 | −0.0249173 | 0.10973154 | 0.30075767 |
| KREMEN1 | | | | | | | | | |
| DKK3)- | −0.022951463 | 0.02459465 | −0.0974743 | −0.1802795 | 0.10660047 | −0.0285048 | −0.0178401 | 0.0088126 | 0.18562551 |
| KREMEN1 | | | | | | | | | |
| BSG)- | −0.021954179 | 0.10511931 | 0.12984884 | 0.09719838 | 0.0909336 | −0.0507766 | −0.2748749 | −0.1978584 | −0.3964063 |
| SELE | | | | | | | | | |
| AFDN)- | 0.069659182 | −0.1486804 | 0.22674603 | −0.0908912 | 0.18646039 | 0.13894366 | −0.0643237 | −0.1109752 | 0.37211734 |
| EPHB3 | | | | | | | | | |
| CXCL12)- | −0.051202162 | 0.04573232 | 0.03941794 | 0.08903713 | 0.1291528 | 0.170609 | 0.01639729 | 0.20355657 | 0.41093872 |
| CCR4 | | | | | | | | | |
| CCL5)- | −0.204623906 | −0.1571002 | 0.18483743 | 0.13746693 | −0.1520628 | 0.21843419 | −0.1854264 | 0.26581376 | 0.34943977 |
| CCR4 | | | | | | | | | |
| NTN4)- | 0.121193785 | −0.0136567 | −0.0293998 | −0.0465074 | −0.1638687 | 0.10490278 | 0.04979938 | −0.1918455 | 0.01058231 |
| UNC5A | | | | | | | | | |
| FAM3C)- | 0.028528115 | −0.1436382 | −0.0031374 | −0.1720288 | −0.1569254 | 0.03602873 | −0.0478134 | −0.0307936 | −0.1901422 |
| LAMP1 | | | | | | | | | |
| POMC)- | −0.075764253 | 0.3019006 | 0.28385318 | 0.06477963 | 0.00041914 | 0.24540445 | 0.13989637 | −0.0071499 | 0.30406445 |
| MC2R | | | | | | | | | |
| F10)- F3 | −0.16064239 | −0.1294634 | 0.01473306 | 0.0450109 | −0.1206409 | −0.0339496 | −0.1193385 | −0.2869141 | 0.05320858 |
| IL6)- F3 | −0.000998436 | 0.09353897 | 0.02425105 | −0.0683921 | −0.0957895 | −0.1090414 | −0.1693979 | −0.1245183 | −0.1870347 |
| PTN)- | −0.057638561 | 0.22378922 | 0.25355012 | 0.17224328 | 0.18298985 | 0.07411381 | 0.18100719 | 0.02789099 | 0.50674404 |
| PTPRB | | | | | | | | | |
| EFNA3)- | 0.077886537 | 0.27691862 | −0.0149832 | 0.12168584 | 0.11252589 | 0.03182585 | 0.04314591 | 0.06624094 | 0.12688137 |
| EPHA3 | | | | | | | | | |
| EFNA1)- | 0.000583067 | 0.16926939 | 0.17163464 | 0.22171121 | 0.1879406 | 0.01175221 | −0.0534262 | −0.1461622 | −0.3232881 |
| EPHA3 | | | | | | | | | |
| EFNA4)- | −0.018712265 | 0.06542004 | 0.1628682 | 0.08244877 | −0.0119395 | 0.22713013 | 0.0697608 | 0.03140936 | −0.0402315 |
| EPHA3 | | | | | | | | | |
| NRG1)- | −0.099868258 | 0.08242991 | 0.0639082 | 0.15700338 | −0.0662251 | 0.14811836 | 0.00497839 | 0.21701004 | 0.34598023 |
| MS4A4A | | | | | | | | | |
| CCN1)- | −0.224521932 | 0.11830754 | 0.30514873 | −0.1979896 | −0.132444 | 0.17665067 | 0.19943164 | −0.1095502 | 0.31074827 |
| CAV1 | | | | | | | | | |
| PTGS2)- | −0.002988985 | −0.0209272 | −0.0797565 | 0.12855371 | −0.0811014 | −0.223217 | −0.0149944 | 0.0714768 | 0.11179527 |
| CAVI | | | | | | | | | |
| HRAS)- | 0.050590917 | −0.0388607 | 0.0140174 | 0.17252258 | 0.25976095 | −0.0751289 | 0.07757494 | −0.2471433 | 0.38677521 |
| CAV1 | | | | | | | | | |
| PLAU)- | 0.073242441 | −0.0511446 | −0.0173695 | 0.01931369 | 0.13633148 | 0.00739928 | 0.05373569 | 0.02963128 | 0.05427455 |
| PLAUR | | | | | | | | | |
| MMP12)- | 0.016702465 | −0.0229085 | 0.14074795 | 0.04282676 | −0.2562062 | 0.10513031 | −0.1625004 | 0.17918369 | 0.16024583 |
| PLAUR | | | | | | | | | |
| FN1)- | −0.195682174 | 0.02580732 | −0.0424747 | 0.08047502 | 0.22957579 | 0.10089914 | −0.0757185 | −0.1250817 | 0.09050431 |
| PLAUR | | | | | | | | | |
| WNT5B)- | −0.05575241 | −0.0583782 | 0.16581628 | 0.15078386 | 0.0318066 | 0.16369471 | −0.1760583 | 0.30006066 | 0.27919934 |
| KLRG2 | | | | | | | | | |
| FGF21)- | −0.051263905 | 0.07631565 | 0.06218412 | −0.0976538 | −0.0133481 | 0.16293438 | −0.0122487 | 0.00098436 | −0.0119488 |
| FGFR3 | | | | | | | | | |
| FGF14)- | −0.215869158 | 0.19948301 | 0.06135122 | −0.059283 | −0.070248 | 0.14603955 | 0.02335599 | −0.0229859 | 0.12608147 |
| FGFR3 | | | | | | | | | |
| FGF17)- | −0.011912234 | 0.17678578 | 0.1695159 | 0.19139733 | −0.0811624 | 0.10752633 | 0.12905226 | 0.03704241 | 0.14703522 |
| FGFR3 | | | | | | | | | |
| FGF12)- | −0.105449269 | 0.09445475 | −0.015443 | 0.14997847 | −0.1776103 | −0.0965172 | 0.0087792 | −0.1308408 | 0.16895985 |
| FGFR3 | | | | | | | | | |
| NRG1)- | −0.072553838 | 0.08141875 | 0.33099371 | 0.0081376 | −0.0715849 | 0.22956736 | 0.05246027 | −0.016089 | 0.09996939 |
| ADGRL1 | | | | | | | | | |
| LAMB3)- | 0.24032293 | 0.01028779 | 0.32833687 | −0.0046635 | 0.25481069 | 0.26897772 | −0.1328187 | −0.242186 | 0.38812686 |
| COL17A1 | | | | | | | | | |
| UCN3)- | −0.095048341 | 0.19981918 | 0.22515609 | 0.06517908 | 0.1020255 | 0.09339537 | 0.27787074 | 0.19715645 | 0.48039183 |
| CRHR2 | | | | | | | | | |
| NPPC)- | 0.125276707 | −0.1315093 | 0.1058432 | −0.0247054 | 0.10561688 | 0.01076879 | 0.11696579 | 0.26968787 | 0.04447459 |
| NPR2 | | | | | | | | | |
| RGMA)- | 0.163126392 | −0.0425418 | 0.09501562 | 0.08705494 | −0.0271745 | −0.09626 | −0.0232783 | 0.09574968 | −0.0795051 |
| NEO1 | | | | | | | | | |
| TNF)- | −0.071394242 | −0.0227311 | 0.17976943 | −0.1340129 | −0.0034161 | 0.13478015 | 0.02857562 | −0.0159315 | −0.1722712 |
| TRADD | | | | | | | | | |
| ADIPOQ)- | 0.158346044 | 0.21968982 | 0.02331872 | 0.15528522 | 0.00359885 | −0.0328056 | 0.23809238 | 0.2069464 | 0.42314233 |
| ADIPOR1 | | | | | | | | | |
| ANXA1)- | −0.072198169 | −0.0292877 | 0.23611493 | 0.06050291 | 0.04602683 | 0.06531397 | −0.1925093 | 0.09455723 | 0.0750874 |
| FPR3 | | | | | | | | | |
| GHRH)- | 0.06836279 | 0.24201481 | 0.15381064 | 0.19443082 | 0.22162936 | 0.02399885 | 0.20285371 | −0.0244645 | 0.50731014 |
| ADRB3 | | | | | | | | | |
| PTHLH)- | −0.109420199 | 0.15575645 | 0.31042265 | −0.0318413 | 0.09277748 | 0.14897889 | 0.24288332 | −0.0340318 | 0.44842834 |
| ADRB3 | | | | | | | | | |
| POMC)- | 0.037704646 | 0.21288507 | 0.23641975 | 0.22897081 | 0.07054942 | 0.20192905 | 0.13505345 | 0.06307469 | 0.48272064 |
| ADRB3 | | | | | | | | | |
| ADCYAP1)- | 0.046056149 | 0.21986777 | 0.34930003 | 0.09213574 | 0.00276066 | 0.17251632 | 0.21094519 | 0.1721377 | 0.52660579 |
| ADRB3 | | | | | | | | | |
| ADM)- | −0.039572589 | 0.22888807 | 0.17691723 | 0.15735254 | 0.00403345 | 0.16580534 | −0.1035993 | 0.0556919 | 0.16509655 |
| ADRB3 | | | | | | | | | |
| GCG)- | −0.15914584 | −0.0873047 | 0.01378789 | 0.20387579 | −0.0173377 | 0.10294525 | 0.11367063 | 0.16515248 | 0.36652069 |
| ADRB3 | | | | | | | | | |
| ADM2)- | 0.018124657 | 0.11844346 | 0.08017008 | 0.09829442 | 0.07841962 | 0.00414693 | −0.0109545 | 0.09622601 | −0.0005273 |
| ADRB3 | | | | | | | | | |
| GNAS)- | −0.164117267 | −0.3183253 | −0.0820162 | −0.2198569 | 0.05564908 | −0.2084661 | −0.2687616 | 0.10368608 | −0.240427 |
| ADRB3 | | | | | | | | | |
| NPS)- | −0.03952217 | 0.22900358 | 0.383042 | 0.08366536 | −0.1251493 | 0.20709609 | 0.35680946 | −0.0441351 | 0.56892776 |
| ADRB3 | | | | | | | | | |
| FSHB)- | −0.059289698 | 0.17753128 | 0.18326784 | 0.28429149 | 0.02639979 | 0.16967151 | 0.22335296 | −0.0088845 | 0.53472789 |
| ADRB3 | | | | | | | | | |
| CALCB)- | −0.050261545 | 0.16786179 | 0.22400165 | 0.26398245 | 0.06807814 | 0.19189179 | 0.09914864 | 0.13005265 | 0.48856829 |
| ADRB3 | | | | | | | | | |
| GIP)- | −0.111037192 | 0.22163072 | 0.36395861 | 0.13558816 | 0.02632157 | 0.23045302 | 0.27230161 | 0.12766725 | 0.49164922 |
| ADRB3 | | | | | | | | | |
| INSL3)- | 0.147412911 | 0.12062712 | 0.08361618 | 0.02734965 | 0.03456564 | 0.16828769 | 0.31047736 | −0.0524484 | 0.51455295 |
| ADRB3 | | | | | | | | | |
| IFNA16)- | −0.050247414 | 0.11195667 | −0.0160335 | −0.1210677 | 0.06571394 | 0.08159402 | 0.04231296 | 0.02874214 | −0.2182084 |
| IFNAR2 | | | | | | | | | |
| IFNA14)- | −0.014741128 | 0.01803896 | −0.1016483 | 0.03831847 | 0.0198068 | 0.11301097 | 0.05730123 | −0.057127 | −0.2994658 |
| IFNAR2 | | | | | | | | | |
| IFNA4)- | 0.038136258 | 0.07255775 | 0.06161485 | −0.1356304 | −0.0219756 | 0.00419167 | 0.10435481 | −0.0404109 | −0.3317481 |
| IFNAR2 | | | | | | | | | |
| IFNA13)- | −0.138194624 | 0.01962922 | −0.0093322 | −0.2178153 | 0.12429197 | 0.11989815 | −0.0472675 | −0.0311359 | −0.2120824 |
| IFNAR2 | | | | | | | | | |
| IFNB1)- | −0.044343621 | −0.0292649 | 0.03762901 | −0.0537627 | −0.1635287 | −0.1562224 | 0.06457831 | 0.04549643 | −0.0935363 |
| IFNAR2 | | | | | | | | | |
| IFNA8)- | −0.026490416 | 0.22843339 | −0.138464 | −0.2472905 | 0.01931502 | 0.00837458 | 0.06612517 | 0.00319032 | −0.3212591 |
| IFNAR2 | | | | | | | | | |
| IFNE)- | 0.050064114 | 0.06721224 | −0.2024417 | −0.0711445 | 0.00968882 | 0.1148916 | 0.02770144 | −0.0448316 | −0.2885193 |
| IFNAR2 | | | | | | | | | |
| IFNA2)- | 0.103053151 | −0.0716832 | 0.08572873 | −0.0325812 | −0.0349933 | 0.02267347 | 0.11492179 | 0.08870071 | −0.1806052 |
| IFNAR2 | | | | | | | | | |
| MDK)- | −0.019344877 | 0.01313512 | −0.0018324 | −0.0278375 | −0.085397 | 0.05058054 | −0.0867983 | −0.0634685 | −0.0008407 |
| SDC3 | | | | | | | | | |
| COL5A1)- | 0.022611932 | 0.04801158 | −0.0515114 | −0.2554318 | −0.1404252 | 0.11872977 | −0.0462736 | −0.0801248 | 0.09560719 |
| SDC3 | | | | | | | | | |
| SHBG)- | 0.142783642 | −0.108265 | 0.19431077 | 0.0392908 | −0.1919735 | 0.15149046 | 0.1740489 | 0.14490743 | −0.0665713 |
| SLC37A1 | | | | | | | | | |
| CD24)- | 0.04004031 | 0.18047549 | −0.0502581 | 0.09381445 | 0.00080594 | −0.0487702 | −0.0689998 | 0.01368952 | −0.1699006 |
| SIGLEC10 | | | | | | | | | |
| LAMA2)- | 0.084690917 | −0.0659478 | 0.12375429 | 0.06489992 | −0.0863334 | 0.01061102 | 0.10632065 | 0.02624094 | −0.3512301 |
| RPSA | | | | | | | | | |
| LAMA1)- | −0.169318884 | −0.125908 | −0.1697331 | 0.0035792 | −0.0254252 | 0.00514684 | 0.03817097 | −0.0157569 | −0.3532802 |
| RPSA | | | | | | | | | |
| LAMB2)- | −0.19045443 | 0.15181656 | −0.2167759 | 0.15834639 | 0.04874247 | 0.11690036 | −0.442557 | −0.367546 | 0.45137818 |
| RPSA | | | | | | | | | |
| EDN1)- | −0.088572576 | 0.29723174 | 0.07688091 | 0.08272723 | 0.02197157 | 0.21907396 | 0.01413481 | −0.0382401 | 0.16339504 |
| KEL | | | | | | | | | |
| NCAM1)- | 0.007391646 | 0.24588138 | 0.22576868 | 0.02232992 | 0.05648469 | 0.13711652 | 0.22289516 | 0.14043228 | 0.51064229 |
| GFRA1 | | | | | | | | | |
| GDNF)- | 0.071656494 | 0.25492066 | 0.16438334 | 0.14144418 | 0.16884032 | 0.07037559 | 0.23792893 | 0.01854749 | 0.37653662 |
| GFRA1 | | | | | | | | | |
| ARTN)- | 0.039036001 | 0.09005927 | 0.17671408 | 0.21282643 | 0.07422614 | 0.23786532 | 0.28427396 | 0.21483187 | 0.39749659 |
| GFRA1 | | | | | | | | | |
| PTPN6)- | −0.131794315 | −0.0391525 | 0.29776349 | 0.0722894 | −0.145397 | 0.18664188 | −0.1913101 | 0.00912709 | 0.16294398 |
| CLEC12A | | | | | | | | | |
| GDNF)- | −0.105639462 | 0.43387414 | 0.20846562 | 0.08367428 | 0.04240194 | 0.16129907 | 0.19236545 | −0.09042 | 0.45443867 |
| RET | | | | | | | | | |
| ARTN)- | 0.033198938 | 0.08688715 | 0.24844204 | 0.19112453 | −0.1219904 | 0.21428111 | 0.19210204 | −0.0038354 | 0.49503258 |
| RET | | | | | | | | | |
| VEGFA)- | −0.063067249 | 0.09790228 | 0.05972907 | 0.2400955 | 0.07113662 | −0.2201074 | −0.0161162 | −0.0299115 | −0.1340871 |
| RET | | | | | | | | | |
| TNFSF14)- | 0.252889269 | −0.1104272 | 0.09464593 | 0.07228822 | −0.0165111 | 0.0682551 | −0.0403982 | 0.0651872 | 0.13561939 |
| TNFRSF14 | | | | | | | | | |
| LTA)- | 0.105474945 | 0.03987613 | 0.06440833 | 0.01350659 | −0.0452624 | 0.16737698 | −0.0866961 | 0.190508 | 0.08984854 |
| TNFRSF14 | | | | | | | | | |
| FASLG)- | 0.196290818 | 0.22243831 | 0.44233209 | 0.14728388 | −0.0473169 | 0.04078207 | 0.19535368 | 0.07358629 | 0.40355153 |
| FAS | | | | | | | | | |
| TNF)- FAS | 0.085184289 | 0.1447399 | 0.32223637 | 0.05840612 | 0.05090234 | 0.31894234 | 0.23594589 | 0.12465365 | 0.35148495 |
| CALM1)- | −0.022884612 | −0.1063568 | −0.013027 | 0.04101356 | 0.05642428 | −0.0956057 | −0.2333918 | 0.00183159 | −0.1180187 |
| FAS | | | | | | | | | |
| PIGA)- | 0.029196089 | −0.1091937 | 0.10936585 | 0.06239875 | −0.0902933 | −0.0674782 | 0.12570566 | 0.20414692 | −0.3303264 |
| PIGR | | | | | | | | | |
| ICAM2)- | −0.065300838 | 0.04260843 | 0.03673534 | 0.13632921 | 0.04264525 | 0.16366439 | 0.11043579 | 0.06350764 | 0.24167442 |
| ITGAL | | | | | | | | | |
| PLAU)- | 0.082829333 | 0.08355316 | 0.1891631 | 0.02023072 | 0.01706209 | 0.12140583 | 0.06819334 | −0.0296781 | −0.0291821 |
| IGF2R | | | | | | | | | |
| IGF2)- | −0.159678218 | −0.1917594 | 0.02899313 | 0.03865961 | 0.10300474 | 0.08731138 | 0.03494325 | −0.1480653 | −0.1700774 |
| IGF2R | | | | | | | | | |
| ADIPOQ)- | 0.03298067 | 0.08465474 | 0.12166478 | 0.14053763 | 0.03854982 | 0.14797672 | 0.10289363 | 0.16943601 | 0.11072736 |
| ADIPOR2 | | | | | | | | | |
| TNF)- | 0.157789791 | −0.0257225 | −0.0740688 | 0.1954746 | 0.01841248 | −0.0795217 | −0.0246181 | −0.0549056 | 0.26434527 |
| PTPRS | | | | | | | | | |
| HSPG2)- | −0.151121413 | −0.0846418 | 0.02443991 | −0.3819571 | −0.2027878 | 0.12581352 | 0.04151559 | −0.1763812 | −0.0818102 |
| PTPRS | | | | | | | | | |
| RARRES2)- | 0.017490277 | −0.0043736 | −0.1173811 | −0.055391 | 0.06581191 | −0.1136848 | −0.0003078 | 0.01297529 | 0.1216375 |
| CCRL2 | | | | | | | | | |
| CCL5)- | −0.054729393 | 0.00108847 | 0.02841726 | 0.01022783 | −0.0998684 | 0.0286607 | 0.00183195 | 0.06931202 | 0.19669307 |
| CCRL2 | | | | | | | | | |
| EFNA3)- | 0.035252312 | −0.1459101 | 0.22094805 | −0.1336853 | 0.18979958 | 0.14777706 | 0.08516441 | 0.08505615 | −0.305204 |
| EPHA2 | | | | | | | | | |
| EFNA1)- | 0.256502042 | −0.2849133 | 0.31453479 | −0.2937022 | 0.37194799 | 0.37076507 | −0.0697621 | 0.00019627 | 0.14141068 |
| EPHA2 | | | | | | | | | |
| EFNA4)- | 0.000124506 | −0.1435335 | 0.05054944 | −0.0683882 | 0.0575537 | 0.05919743 | −0.0989498 | −0.0579188 | −0.0130502 |
| EPHA2 | | | | | | | | | |
| SEMA4D)- | −0.238255128 | −0.1099815 | −0.088303 | −0.155497 | 0.13065519 | 0.18344468 | 0.02167125 | 0.10612223 | 0.05333708 |
| PLXNB1 | | | | | | | | | |
| SEMA4A)- | −0.07783691 | 0.03946093 | −0.0691948 | −0.1331779 | 0.08734888 | 0.10862805 | 0.14445196 | 0.0481033 | −0.1070699 |
| PLXNB1 | | | | | | | | | |
| TGFB2)- | 0.124815984 | −0.167579 | 0.22671487 | 0.01248596 | 0.11932832 | 0.04247347 | −0.1755179 | −0.1421926 | 0.12959903 |
| TGFBR2 | | | | | | | | | |
| S100A10)- | −0.051369186 | 0.18209354 | 0.15053159 | 0.19208258 | 0.22219488 | −0.1485297 | 0.0370359 | −0.0990384 | −0.4058598 |
| TRPV6 | | | | | | | | | |
| ADM)- | 0.040785758 | 0.17068908 | 0.18999201 | 0.30742634 | 0.13955527 | 0.14341476 | 0.15999297 | 0.17819567 | 0.273268 |
| CALCR | | | | | | | | | |
| ADM2)- | 0.085718767 | 0.10643312 | 0.08914697 | −0.054084 | 0.03755902 | 0.20905702 | 0.07145379 | −0.0267387 | −0.0598346 |
| CALCR | | | | | | | | | |
| CALCB)- | 0.073501239 | 0.13864303 | 0.14012355 | 0.23779492 | 0.05230441 | 0.19385185 | 0.21184876 | 0.23062881 | 0.408875 |
| CALCR | | | | | | | | | |
| MBL2)- | 0.06015106 | 0.28149102 | 0.19771643 | 0.16901448 | 0.03678584 | 0.19580623 | 0.12084044 | 0.30510702 | 0.51803371 |
| CALCR | | | | | | | | | |
| GSTO1)- | −0.071454534 | 0.04000188 | −0.0432396 | −0.1517416 | 0.00817908 | −0.0540347 | −0.0991764 | −0.1326703 | −0.2487646 |
| RYR1 | | | | | | | | | |
| TNFSF13B)- | 0.07188235 | −0.1250868 | 0.04015381 | −0.2660663 | 0.10679091 | −0.0962955 | −0.1357973 | 0.12283192 | −0.1477983 |
| TFRC | | | | | | | | | |
| B2M)- | −0.033740008 | −0.04597 | 0.34529571 | 0.13067906 | −0.0502714 | 0.07200926 | −0.1291029 | −0.115449 | 0.11652581 |
| TFRC | | | | | | | | | |
| VEGFC)- | −0.126121167 | 0.07716648 | 0.03545104 | 0.1526429 | 0.07332501 | −0.0931046 | 0.0884877 | −0.1039865 | 0.1385167 |
| NRP2 | | | | | | | | | |
| SEMA3B)- | −0.180665015 | −0.0546014 | −0.0663775 | 0.10085247 | −0.1327385 | −0.0522899 | 0.10128057 | 0.26330831 | 0.17221657 |
| NRP2 | | | | | | | | | |
| VEGFA)- | −0.153654095 | 0.34804069 | 0.01322184 | 0.39842694 | 0.11950595 | −0.1273211 | −0.0582551 | 0.01993591 | −0.195149 |
| NRP2 | | | | | | | | | |
| PGF)- | 0.020483139 | 0.00322375 | 0.04834506 | 0.1676251 | −0.0344362 | −0.1697907 | −0.0543197 | 0.00414104 | 0.07947272 |
| NRP2 | | | | | | | | | |
| CXCL2)- | −0.047537141 | −0.048633 | −0.0013908 | 0.15661034 | −0.0511084 | 0.0172145 | 0.17277197 | 0.11006922 | 0.27811538 |
| DPP4 | | | | | | | | | |
| ADCYAP1)- | 0.103450168 | 0.16229252 | 0.35564579 | 0.14370448 | 0.05309178 | 0.28815126 | 0.26670333 | 0.14231616 | 0.55158515 |
| DPP4 | | | | | | | | | |
| GCG)- | −0.08307769 | 0.0003534 | 0.07142974 | 0.166788 | 0.09062173 | 0.07975973 | 0.27150091 | 0.27280572 | 0.50554743 |
| DPP4 | | | | | | | | | |
| CXCL9)- | 0.18878073 | 0.05442289 | −0.0052755 | 0.18385319 | 0.15574177 | 0.01790835 | 0.16555007 | 0.31907323 | 0.37466368 |
| DPP4 | | | | | | | | | |
| CXCL12)- | 0.098189846 | 0.06141997 | 0.05382018 | 0.06775564 | 0.01739936 | 0.09028841 | 0.05459736 | 0.21402088 | 0.35633593 |
| DPP4 | | | | | | | | | |
| NPY)- | 0.217873125 | 0.3302451 | 0.27107021 | 0.17719173 | 0.0674509 | 0.13167936 | 0.29353653 | 0.20584302 | 0.44012825 |
| DPP4 | | | | | | | | | |
| ADA)- | 0.211822574 | 0.16269054 | 0.08478045 | 0.18330141 | 0.02963933 | 0.1977108 | 0.13883823 | 0.21289905 | 0.36941595 |
| DPP4 | | | | | | | | | |
| MMP9)- | −0.011717788 | 0.01520248 | −0.0470684 | 0.24890183 | 0.05158004 | 0.06222782 | 0.28120176 | −0.0257741 | 0.38147436 |
| TLR9 | | | | | | | | | |
| HRAS)- | 0.059540862 | 0.11638429 | 0.01473363 | 0.14603608 | 0.02059634 | 0.05118088 | 0.06584134 | 0.06727828 | 0.30271242 |
| TLR9 | | | | | | | | | |
| B2M)- | −0.05122706 | −0.0455219 | −0.0148025 | −0.0492393 | 0.03517244 | −0.0492084 | 0.12752594 | 0.03155838 | −0.0422176 |
| CD247 | | | | | | | | | |
| COL4A4)- | −0.104536765 | 0.0563323 | −0.0700981 | −0.0664488 | −0.0868637 | −0.092637 | 0.02458174 | 0.1299362 | −0.2121773 |
| CD47 | | | | | | | | | |
| COL4A3)- | 0.1437044 | −0.0358031 | 0.23577107 | −0.0761118 | −0.1501764 | −0.0317905 | 0.10467216 | 0.10572671 | −0.1955896 |
| CD47 | | | | | | | | | |
| THBS2)- | 0.145067642 | −0.1461093 | 0.14726691 | 0.35279764 | −0.3130334 | −0.4019839 | 0.15833779 | 0.19276125 | −0.2268978 |
| CD47 | | | | | | | | | |
| CD55)- CR1 | −0.108528074 | 0.02162135 | 0.24402654 | 0.07439033 | 0.23573108 | −0.0965345 | −0.3001457 | 0.0249633 | −0.1895622 |
| C4B)- CR1 | 0.068310445 | 0.02289753 | −0.1745941 | 0.07266991 | 0.20200589 | −0.0175918 | 0.07460064 | 0.21240111 | 0.31737825 |
| C1QA)- | −0.019918107 | −0.151078 | −0.1018542 | 0.13230573 | 0.1194235 | −0.023791 | 0.22615449 | −0.0262499 | 0.22749695 |
| CR1 | | | | | | | | | |
| AFDN)- | −0.011732791 | −0.1189584 | 0.11916985 | −0.2216953 | 0.04980988 | 0.01529042 | −0.0649189 | 0.0740725 | 0.2646837 |
| NECTIN4 | | | | | | | | | |
| AFDN)- | 0.301012554 | −0.2596814 | 0.30045011 | −0.2201036 | 0.22563721 | 0.30383659 | −0.0729227 | −0.0125375 | −0.0535276 |
| F11R | | | | | | | | | |
| B2M)- | −0.102167607 | 0.12210344 | 0.07691001 | 0.05760544 | 0.28693144 | −0.1294921 | 0.21425895 | 0.01346215 | −0.051442 |
| CD1B | | | | | | | | | |
| IL6)- IL6R | −0.137769437 | 0.13300197 | 0.24200992 | 0.03190999 | −0.0936885 | 0.30408115 | 0.06933866 | 0.06510105 | 0.36141206 |
| CD58)- | 0.021432139 | −0.0122627 | 0.0745399 | 0.03950248 | 0.0550897 | 0.05617561 | −0.0115546 | 0.07751391 | 0.05861882 |
| CD2 | | | | | | | | | |
| CD59)- | 0.177771073 | 0.15549111 | −0.237597 | 0.14839356 | −0.1633202 | 0.12581583 | −0.0460305 | −0.005721 | −0.0747797 |
| CD2 | | | | | | | | | |
| TNFSF15)- | −0.056297228 | 0.00746413 | 0.11927413 | 0.25852282 | −0.16998 | −0.0066965 | 0.01129862 | 0.00705104 | 0.12257799 |
| TNFRSF6B | | | | | | | | | |
| TNFSF14)- | 0.121265246 | −0.0971989 | 0.19358581 | 0.10733428 | −0.0596212 | 0.06849989 | 0.19514632 | 0.14192727 | 0.10762944 |
| TNFRSF6B | | | | | | | | | |
| EDN1)- | −0.000570305 | 0.17500375 | 0.14854127 | 0.11696606 | −0.2102278 | 0.21344361 | 0.25930595 | −0.083003 | 0.24248478 |
| ADGRL4 | | | | | | | | | |
| MDK)- | 0.173273555 | −0.077226 | 0.13943045 | 0.05431595 | 0.03427922 | 0.08381472 | −0.0553028 | −0.0417548 | 0.16230538 |
| TSPAN1 | | | | | | | | | |
| BMP7)- | 0.018573878 | 0.2049107 | 0.16309291 | 0.20313597 | 0.11275866 | 0.03632908 | −0.0918392 | −0.032575 | 0.13166166 |
| BMPR1A | | | | | | | | | |
| BMP2)- | 0.116851829 | 0.17928173 | 0.22979791 | 0.16026963 | 0.03692136 | −0.0051951 | −0.0184377 | −0.1058966 | 0.04923911 |
| BMPR1A | | | | | | | | | |
| TNFSF13B)- | 0.052543053 | 0.10830655 | 0.14301392 | 0.20569835 | −0.002223 | 0.12104624 | 0.06628483 | 0.19435231 | 0.17348663 |
| CD40 | | | | | | | | | |
| LTB)- | 0.017139315 | −0.046576 | 0.10183136 | 0.16318382 | −0.168764 | 0.1222424 | 0.043002 | 0.10359464 | 0.19853797 |
| CD40 | | | | | | | | | |
| ANGPTL2)- | 0.012298031 | 0.09307164 | 0.0028496 | 0.01292592 | 0.05981784 | −0.0028599 | 0.06711358 | 0.03513321 | 0.29581003 |
| TIE1 | | | | | | | | | |
| ANGPT1)- | 0.161580845 | 0.35820661 | 0.16855579 | 0.04098188 | 0.07439773 | 0.08490283 | 0.19610752 | 0.0919234 | 0.32806541 |
| TIE1 | | | | | | | | | |
| NTF4)- | −0.229868658 | 0.17663637 | 0.06831095 | −0.0530443 | −0.0987492 | −0.0300214 | −0.0541488 | −0.1058317 | 0.26894412 |
| BEX3 | | | | | | | | | |
| BDNF)- | −0.170388022 | 0.0532494 | 0.13458874 | 0.17084046 | −0.0835422 | 0.02974367 | 0.03393827 | −0.1456238 | 0.33183694 |
| BEX3 | | | | | | | | | |
| MDK)- | 0.248609301 | −0.0880734 | 0.28863859 | −0.1436288 | 0.17341416 | 0.22405664 | −0.1110566 | −0.0784884 | 0.34145734 |
| SDC4 | | | | | | | | | |
| CXCL12)- | 0.182178708 | −0.113357 | 0.2557325 | −0.2214942 | −0.0961269 | −0.1309202 | 0.11265324 | 0.04443793 | −0.329304 |
| SDC4 | | | | | | | | | |
| CCL5)- | −0.295181735 | 0.00891579 | −0.1888985 | −0.1796305 | 0.16486634 | 0.20947144 | 0.22696122 | 0.21829042 | −0.3097884 |
| SDC4 | | | | | | | | | |
| LAMA1)- | 0.237179726 | −0.2097935 | 0.24011033 | −0.2002227 | −0.1370701 | −0.1791208 | 0.14477648 | 0.06074853 | −0.4445564 |
| SDC4 | | | | | | | | | |
| BMP7)- | −0.094916587 | 0.16436575 | −0.0670646 | 0.14872319 | 0.01647347 | −0.0770021 | −0.1782087 | 0.16874642 | 0.04065617 |
| ENG | | | | | | | | | |
| BMP2)- | 0.029000926 | 0.10771731 | −0.0598016 | 0.05248626 | −0.0179473 | −0.0388014 | 0.00173135 | 0.16310113 | 0.11344123 |
| ENG | | | | | | | | | |
| CCN1)- | −0.019373975 | 0.09263794 | −0.1507588 | −0.036229 | 0.13718177 | 0.03258744 | −0.1109548 | 0.04098863 | 0.20740535 |
| TLR4 | | | | | | | | | |
| HSPA1A)- | 0.021857043 | 0.00417293 | −0.0530105 | 0.04130391 | −0.0524772 | −0.0420026 | −0.020884 | 0.07240836 | 0.00857123 |
| TLR4 | | | | | | | | | |
| IRAK4)- | −0.099008116 | −0.1527745 | 0.22632243 | 0.11281565 | −0.0142431 | 0.1009939 | 0.14778831 | 0.00623972 | 0.07971913 |
| TLR4 | | | | | | | | | |
| ZG16B)- | 0.157444478 | 0.28827649 | −0.0128765 | 0.22370878 | 0.0105922 | 0.06218388 | 0.00429414 | −0.1958367 | −0.2313452 |
| TLR4 | | | | | | | | | |
| NPY)- | −0.064809279 | 0.09677835 | 0.06169333 | −0.0674799 | −0.0946101 | 0.04101397 | 0.06880678 | −0.0698187 | −0.1732483 |
| NPY4R | | | | | | | | | |
| PPY)- | 0.002238468 | 0.09576577 | 0.13150349 | 0.09880982 | −0.1623027 | −0.0023107 | 0.19705504 | −0.0377103 | 0.06302922 |
| NPY4R | | | | | | | | | |
| ALOX5AP)- | −0.266100552 | −0.396649 | 0.56372877 | 0.31037114 | −0.3773789 | 0.34449009 | −0.4161325 | 0.37080554 | 0.27456529 |
| ALOX5 | | | | | | | | | |
| ADIPOQ)- | 0.065670924 | 0.21686539 | 0.22299585 | 0.11653057 | 0.07446895 | 0.11390901 | 0.31988635 | 0.0493283 | 0.59583829 |
| CNR2 | | | | | | | | | |
| BMP7)- | −0.114816217 | −0.0759476 | −0.052564 | −0.07027 | −0.1389023 | 0.01815896 | −0.1892023 | −0.0202554 | −0.0217708 |
| BMPR2 | | | | | | | | | |
| BMP2)- | 0.11700948 | −0.2390637 | −0.121343 | −0.1127388 | −0.2061636 | 0.03043758 | 0.04056134 | 0.08523747 | −0.0886709 |
| BMPR2 | | | | | | | | | |
| RGMA)- | 0.022423778 | −0.0488627 | 0.01697291 | −0.1521793 | −0.1009289 | −0.0773331 | −0.1809527 | 0.06889298 | −0.0639912 |
| BMPR2 | | | | | | | | | |
| VEGFA)- | 0.106606853 | 0.17791865 | 0.10708253 | 0.2154313 | 0.10650759 | 0.06299268 | −0.1591491 | −0.0640105 | −0.0396223 |
| EPHB2 | | | | | | | | | |
| AFDN)- | 0.203940083 | 0.19504193 | 0.07795041 | 0.30100126 | 0.11479697 | 0.11719767 | −0.0602613 | −0.140792 | −0.2649756 |
| EPHB2 | | | | | | | | | |
| PLTP)- | −0.051094123 | −0.1687993 | 0.23457976 | 0.16521939 | −0.1554605 | 0.21998222 | 0.00766142 | −0.0548449 | 0.20383156 |
| ABCA1 | | | | | | | | | |
| HLA-C)- | 0.057932864 | −0.1286381 | 0.10800706 | 0.00602269 | 0.06997959 | 0.01777487 | 0.0965312 | −0.1219399 | −0.1085698 |
| NOTCH4 | | | | | | | | | |
| LTA)- | −0.021711064 | 0.02182064 | 0.01738951 | 0.2838777 | −0.1598414 | 0.26097498 | −0.2324131 | 0.12312447 | 0.19607148 |
| TNFRSF1B | | | | | | | | | |
| TNF)- | 0.116856122 | 0.05484699 | −0.0761741 | 0.25412042 | 0.02304722 | 0.1638789 | −0.1517157 | 0.14571558 | 0.1877402 |
| TNFRSF1B | | | | | | | | | |
| GDNF)- | 0.101080837 | 0.27816612 | 0.17026664 | 0.08751648 | 0.02710803 | 0.01667519 | 0.01790738 | 0.15540927 | 0.3820778 |
| EDNRB | | | | | | | | | |
| EDN1)- | 0.102982711 | 0.00925481 | 0.00323956 | 0.08025454 | −0.019788 | 0.17366137 | −0.0969794 | −0.0071192 | 0.13602343 |
| EDNRB | | | | | | | | | |
| NPNT)- | 0.047447807 | 0.14409431 | 0.00211459 | 0.15504682 | −0.2355464 | 0.02927082 | 0.06164124 | 0.00892461 | −0.0549047 |
| ITGA8 | | | | | | | | | |
| NRG1)- | −0.058795605 | 0.00871175 | 0.19598196 | −0.1739271 | 0.14439182 | 0.16543461 | 0.24560579 | 0.14599735 | −0.2104414 |
| LGR4 | | | | | | | | | |
| TNFSF11)- | 0.041475648 | −0.0320443 | 0.26231822 | −0.0817887 | 0.1069488 | 0.13596063 | 0.19887362 | 0.10734865 | 0.07450512 |
| LGR4 | | | | | | | | | |
| COL8A2)- | 0.012530807 | −0.0167761 | 0.12719737 | −0.0282546 | 0.02117233 | 0.04797982 | 0.19545452 | 0.29538044 | −0.0221692 |
| SLC4A11 | | | | | | | | | |
| NMB)- | −0.196608591 | 0.06045857 | 0.12840765 | 0.13818388 | −0.1420215 | 0.36119162 | 0.13241968 | 0.11339977 | 0.18025017 |
| GRPR | | | | | | | | | |
| SPTBN2)- | −0.041005749 | 0.09214703 | 0.06272609 | −0.0956303 | −0.0525734 | 0.07987754 | 0.06487003 | −0.0111859 | 0.19121606 |
| PTPRA | | | | | | | | | |
| NCAM1)- | −0.038809089 | −0.1340863 | 0.06924684 | 0.03221742 | 0.05417304 | −0.0132546 | 0.02995304 | 0.01549261 | 0.12195293 |
| PTPRA | | | | | | | | | |
| IRAK4)- | 0.04096674 | 0.08350518 | −0.0064617 | 0.00626053 | −0.0457299 | 0.22553692 | 0.09044515 | 0.01854361 | 0.17172661 |
| TLR7 | | | | | | | | | |
| MDK)- | 0.096544178 | −0.1256983 | 0.11172402 | −0.0882616 | 0.13846481 | 0.26319906 | −0.1144453 | −0.0993432 | 0.14886627 |
| SDC1 | | | | | | | | | |
| CCL5)- | −0.119470292 | −0.0203836 | −0.1107698 | −0.0022703 | 0.09088858 | 0.09408818 | 0.21931424 | 0.04169596 | −0.0670761 |
| SDC1 | | | | | | | | | |
| SLIT2)- | −0.206683524 | −0.0819325 | −0.1124141 | 0.0988458 | −0.0443474 | 0.04363631 | 0.01606808 | −0.2221633 | −0.1118832 |
| SDC1 | | | | | | | | | |
| IL6)- IL6ST | −0.030721199 | 0.18271362 | 0.05720965 | 0.05401074 | −0.2451856 | 0.09586204 | −0.2051833 | 0.02087258 | 0.11434639 |
| NPS)- | −0.071158672 | 0.00689567 | 0.33864449 | 0.09835088 | 0.05188496 | 0.07390014 | 0.16426846 | 0.05030811 | 0.28052265 |
| NPSR1 | | | | | | | | | |
| B2M)- | 0.020763912 | −0.1577413 | 0.15987422 | 0.00039839 | 0.14883579 | −0.041793 | 0.14413572 | −0.1307558 | −0.0185426 |
| KLRC2 | | | | | | | | | |
| HLA-G)- | −0.000331022 | −0.1856272 | 0.20372416 | −0.0712235 | 0.20888173 | 0.12567374 | 0.13246137 | −0.1451596 | −0.1339754 |
| KLRD1 | | | | | | | | | |
| B2M)- | 0.024465692 | −0.1875117 | 0.27234273 | −0.0468253 | 0.16511778 | 0.1136344 | 0.05892933 | −0.1813543 | −0.2012209 |
| KLRD1 | | | | | | | | | |
| HLA-B)- | −0.043667841 | −0.2739031 | 0.11405024 | −0.0926048 | 0.09962373 | 0.04783003 | 0.11877554 | −0.1249766 | −0.120149 |
| KLRD1 | | | | | | | | | |
| HLA-G)- | 0.042427598 | −0.0717471 | 0.15803259 | −0.1249537 | 0.11995688 | 0.1084727 | 0.23620474 | −0.0345405 | −0.1498267 |
| KIR3DL1 | | | | | | | | | |
| B2M)- | −0.044342306 | −0.1827143 | 0.19416344 | −0.2014213 | 0.17790675 | −0.0405488 | 0.14142284 | 0.04073036 | −0.1654312 |
| KIR3DL1 | | | | | | | | | |
| HLA-B)- | −0.081186184 | −0.2004491 | 0.09407094 | −0.1524574 | 0.08374288 | 0.03292063 | 0.1680158 | 0.02769453 | −0.1484294 |
| KIR3DL1 | | | | | | | | | |
| HLA-G)- | −0.05855229 | −0.1137927 | 0.15564195 | −0.038093 | −0.0022564 | 0.03205395 | −0.0056526 | −0.0806212 | −0.0469102 |
| LILRB2 | | | | | | | | | |
| B2M)- | −0.222517445 | −0.1755018 | 0.28214808 | −0.0854866 | −0.0415465 | −0.1017836 | −0.1185705 | 0.03346301 | −0.111622 |
| LILRB2 | | | | | | | | | |
| INS)- | 0.01413704 | 0.13712533 | −0.0915351 | 0.11487246 | 0.14463975 | 0.11714893 | 0.06663473 | 0.05256019 | 0.27285869 |
| LILRB2 | | | | | | | | | |
| HLA-B)- | −0.187216665 | −0.2723627 | 0.11429041 | 0.00226437 | −0.1311257 | −0.02952 | −0.0214765 | −0.0347813 | −0.0492918 |
| LILRB2 | | | | | | | | | |
| MDK)- | 0.065158826 | −0.0273073 | 0.04055388 | 0.00678437 | 0.08405393 | −0.0300759 | −0.1195686 | −0.2995664 | −0.056041 |
| PTPRZ1 | | | | | | | | | |
| CCL5)- | 0.041181151 | 0.17762412 | 0.05904311 | −0.0736613 | −0.0285676 | 0.16038778 | 0.01384265 | −0.0420691 | 0.11509613 |
| GPR75 | | | | | | | | | |
| TNF)- VSIR | 0.019460607 | 0.02405815 | −0.0188298 | 0.15116468 | −0.0060112 | 0.22039269 | −0.1444827 | −0.033219 | −0.0233351 |
| VEGFC)- | −0.201548414 | 0.08411449 | −0.0638548 | 0.06280995 | 0.06820713 | 0.02150431 | 0.00014354 | −0.3220455 | 0.00174882 |
| ITGB1 | | | | | | | | | |
| LAMA2)- | −0.175348391 | 0.11110574 | 0.03809577 | −0.1518246 | −0.1159269 | 0.08203467 | 0.03280259 | −0.3176974 | 0.05551666 |
| ITGB1 | | | | | | | | | |
| LAMB2)- | −0.129112107 | 0.31608973 | 0.33398902 | −0.2425154 | −0.1192591 | 0.57279161 | 0.27801937 | −0.2074306 | 0.48749323 |
| ITGB1 | | | | | | | | | |
| NPNT)- | −0.084664987 | 0.12030533 | 0.02955948 | −0.0847753 | −0.0286451 | −0.180122 | −0.0795254 | 0.09671282 | −0.1336919 |
| ITGB1 | | | | | | | | | |
| DSPP)- | −0.081329138 | −0.1925463 | −0.2630189 | −0.1408024 | 0.04961423 | −0.0326268 | −0.1332785 | −0.1705793 | −0.2282885 |
| ITGB1 | | | | | | | | | |
| MDK)- | 0.091996549 | −0.068895 | 0.01050751 | −0.0415264 | 0.08655329 | 0.22608314 | 0.17923079 | −0.0048774 | 0.11196778 |
| ITGB1 | | | | | | | | | |
| COL4A4)- | −0.038914969 | −0.0627388 | −0.2076089 | −0.1482866 | 0.02194882 | −0.0639253 | −0.154051 | −0.2877006 | −0.0994981 |
| ITGB1 | | | | | | | | | |
| COL4A3)- | −0.055542215 | −0.209059 | −0.1492094 | 0.00694814 | −0.1000571 | −0.1663217 | −0.0509733 | −0.1487766 | −0.1530784 |
| ITGB1 | | | | | | | | | |
| ADAM9)- | −0.072428531 | 0.11002302 | 0.19750157 | 0.03132351 | 0.13085374 | −0.006096 | 0.01452954 | 0.22992061 | 0.10971577 |
| ITGB1 | | | | | | | | | |
| ANGPT1)- | −0.132462502 | −0.1370528 | −0.2103902 | −0.1804 | −0.1603029 | −0.0027611 | −0.0146671 | −0.2699665 | −0.1186525 |
| ITGB1 | | | | | | | | | |
| VEGFA)- | −0.151059853 | 0.2843651 | 0.23270543 | 0.25940871 | 0.18829843 | −0.0955299 | 0.03818083 | 0.28140855 | −0.0385295 |
| ITGB1 | | | | | | | | | |
| SEMA4D)- | 0.101319952 | −0.0539544 | −0.1327238 | 0.21034392 | 0.02388765 | −0.0281066 | 0.04372389 | −0.1492997 | 0.06202554 |
| CD72 | | | | | | | | | |
| MDK)- | −0.129399565 | 0.01473455 | −0.0160601 | 0.03127664 | −0.0294294 | −0.0377335 | 0.03555255 | −0.0487126 | −0.2648144 |
| ITGA4 | | | | | | | | | |
| IL6)- HRH1 | 0.033837799 | −0.0125174 | 0.31721448 | 0.03794301 | −0.2272797 | 0.2309952 | 0.12087518 | 0.26572991 | 0.15728897 |
| CCN1)- | 0.271662187 | 0.26464893 | −0.1783349 | −0.1335437 | 0.14650326 | −0.1509941 | −0.1888151 | 0.29530825 | 0.36158957 |
| ITGB2 | | | | | | | | | |
| SPON2)- | 0.02939001 | 0.02623215 | 0.00873702 | −0.2825789 | 0.26878055 | −0.1946378 | −0.2152751 | 0.32450776 | 0.33630952 |
| ITGB2 | | | | | | | | | |
| ICAM2)- | −0.053616532 | −0.0832917 | 0.04136115 | 0.08177133 | −0.0508393 | 0.04332067 | −0.220085 | 0.19905235 | 0.29787945 |
| ITGB2 | | | | | | | | | |
| TNF)- | −0.12182313 | 0.07726728 | 0.0737611 | 0.28430169 | −0.1240881 | 0.26008861 | 0.11069152 | 0.056124 | 0.34220189 |
| TRPM2 | | | | | | | | | |
| VEGFA)- | −0.122130785 | 0.0927261 | 0.02903007 | 0.11165809 | 0.02449007 | 0.04389459 | 0.02522993 | 0.02506863 | −0.1774749 |
| SIRPA | | | | | | | | | |
| GCG)- | −0.047164395 | −0.0774625 | 0.11280953 | 0.1819865 | 0.02526512 | 0.25123434 | 0.18945325 | 0.12940522 | 0.25261114 |
| GCGR | | | | | | | | | |
| IFNE)- | 0.16948231 | 0.25144405 | 0.11795296 | 0.0591432 | 0.06682216 | 0.03714897 | −0.0748066 | 0.12041293 | 0.09143917 |
| ADGRV1 | | | | | | | | | |
| FSHB)- | 0.015957869 | 0.01340915 | 0.19712261 | 0.31337641 | 0.00295693 | 0.17374421 | 0.08876721 | 0.06693085 | 0.50452068 |
| FSHR | | | | | | | | | |
| SYTL3)- | 0.117220408 | −0.0143264 | 0.03785685 | 0.21745142 | −0.0144552 | 0.12912086 | 0.02700113 | 0.24374603 | 0.34480314 |
| NRXN1 | | | | | | | | | |
| LAMA2)- | 0.053201651 | −0.0446644 | 0.06257105 | 0.05732178 | −0.0454916 | −0.1698259 | 0.09798301 | 0.16460759 | −0.2026481 |
| ITGA6 | | | | | | | | | |
| ADAM9)- | 0.334368853 | −0.0839841 | 0.2884281 | −0.14959 | 0.26875548 | 0.37057651 | −0.0847283 | −0.1839129 | 0.06677447 |
| ITGA6 | | | | | | | | | |
| ADM)- | −0.154512264 | 0.1455862 | 0.11737966 | 0.27893369 | 0.07974499 | 0.0812412 | −0.0167423 | −0.0528229 | 0.20930032 |
| CALCRL | | | | | | | | | |
| CALCB)- | 0.022530721 | 0.13474267 | 0.10653141 | 0.22907685 | −0.1336312 | 0.13187166 | −0.0008658 | 0.18212113 | 0.52832593 |
| CALCRL | | | | | | | | | |
| IRAK4)- | −0.093513995 | 0.05453337 | 0.1280122 | 0.12254464 | −0.047594 | 0.25041575 | −0.0067726 | 0.17804449 | 0.13003919 |
| TLR6 | | | | | | | | | |
| FGF21)- | 0.101795842 | 0.18346416 | 0.03990446 | −0.1832131 | −0.2523499 | 0.10101303 | −0.1359768 | 0.02674538 | 0.03970425 |
| FGFR1 | | | | | | | | | |
| NCAM1)- | −0.029458609 | 0.06320222 | −0.0224831 | 0.00422512 | 0.0116706 | −0.0910013 | −0.1898142 | −0.0219201 | 0.1383669 |
| FGFR1 | | | | | | | | | |
| FGF12)- | −0.131907172 | 0.16251978 | −0.0848324 | −0.1037657 | −0.0526078 | 0.12658267 | −0.0159915 | −0.1184642 | 0.22217503 |
| FGFR1 | | | | | | | | | |
| PRG4)- | −0.162411934 | −0.0135942 | −0.0721779 | −0.0566231 | −0.1807666 | −0.0372694 | −0.1968608 | −0.1683728 | −0.1735777 |
| CD44 | | | | | | | | | |
| VIM)- | −0.111505107 | −0.1171437 | 0.32379018 | 0.14512215 | 0.12211138 | 0.26664929 | −0.3182838 | −0.0124438 | 0.45269146 |
| CD44 | | | | | | | | | |
| RGMA)- | 0.079352149 | 0.29529258 | 0.09998579 | 0.04426812 | 0.19471478 | 9.97E−05 | 0.12336841 | 0.07549648 | 0.44219536 |
| BMPR1B | | | | | | | | | |
| SEMA4D)- | −0.118580452 | 0.2301509 | −0.154655 | −0.1134051 | 0.03410253 | 0.13689986 | 0.12555111 | 0.21473203 | −0.139814 |
| PLXNB2 | | | | | | | | | |
| FGF21)- | −0.01155406 | 0.17722801 | 0.00760684 | −0.1010661 | −0.120988 | −0.0616366 | 0.03138385 | 0.05926382 | 0.22725255 |
| FGFR2 | | | | | | | | | |
| NCAM1)- | −0.005984777 | 0.3537337 | 0.20844644 | −0.0940648 | 0.05091538 | 0.14610843 | 0.18637421 | 0.07924088 | 0.28620328 |
| FGFR2 | | | | | | | | | |
| FGF12)- | −0.02244161 | 0.13234166 | 0.18439947 | 0.25910103 | −0.0023923 | 0.19772918 | 0.04497879 | 0.01363656 | 0.27521681 |
| FGFR2 | | | | | | | | | |
| CXCL17)- | 0.192233365 | −0.188972 | 0.22630973 | −0.2422127 | 0.15047375 | 0.15093599 | −0.1209143 | −0.0650039 | 0.13267286 |
| GPR35 | | | | | | | | | |
| B2M)- HFE | −0.006478219 | 0.02927568 | −0.0055128 | 0.03752765 | 0.04989699 | −0.0418463 | 0.16050834 | −0.0626111 | −0.0252889 |
| RGMA)- | 0.030034052 | 0.24436313 | 0.20387101 | −0.0127049 | 0.09412263 | 0.10262963 | 0.21697318 | 0.14595771 | 0.42801841 |
| HFE | | | | | | | | | |
| NCAM1)- | 0.065235045 | 0.11580334 | 0.15991534 | 0.09282081 | 0.00935289 | 0.07800003 | 0.14999672 | 0.31617227 | 0.36363823 |
| ROBO1 | | | | | | | | | |
| SLIT2)- | −0.080546831 | 0.07910045 | 0.15304493 | −0.1110597 | −0.0835218 | 0.3153391 | 0.03127414 | −0.0334947 | 0.41992407 |
| ROBO1 | | | | | | | | | |
| RGMA)- | 0.020709279 | 0.30182129 | 0.20691106 | −0.0381147 | −0.0882101 | 0.09015136 | 0.16892474 | 0.11048068 | 0.45740767 |
| TFR2 | | | | | | | | | |
| B2M)- | −0.096778873 | −0.120108 | 0.17407879 | −0.1417706 | 0.23783356 | −0.1467228 | 0.24850683 | −0.0427189 | −0.1655301 |
| CD3G | | | | | | | | | |
| CCL5)- | 0.036477678 | 0.01389666 | 0.09783606 | 0.01986387 | −0.0290817 | 0.00767581 | 0.06522131 | 0.08336556 | 0.2831369 |
| CCR3 | | | | | | | | | |
| CCL14)- | 0.171725722 | 0.15595425 | 0.02473068 | 0.11461749 | 0.0968078 | 0.1881219 | 0.26233303 | 0.29802525 | 0.52905456 |
| CCR3 | | | | | | | | | |
| CCL15)- | 0.067672043 | 0.10824452 | −0.0724072 | 0.14912281 | −0.0587636 | 0.08858265 | 0.06637972 | 0.18826878 | 0.21738876 |
| CCR3 | | | | | | | | | |
| CCL18)- | −0.011042382 | 0.179021 | 0.12791527 | −0.0271243 | 0.02506747 | 0.20993896 | 0.22121041 | 0.13496751 | 0.30440673 |
| CCR3 | | | | | | | | | |
| SPON2)- | −0.157961343 | −0.1533362 | −0.0078737 | −0.0920019 | 0.11571562 | 0.00308344 | −0.0788898 | −0.0352821 | 0.03991212 |
| ITGAM | | | | | | | | | |
| ADCYAP1)- | −0.001309191 | 0.12925148 | 0.06551886 | 0.20086605 | −0.1234048 | 0.28935498 | 0.00869895 | 0.20903402 | 0.46683315 |
| GPR84 | | | | | | | | | |
| VEGFA)- | 0.040420563 | 0.0071887 | 0.12654768 | 0.05712269 | 0.14497017 | 0.06744888 | 0.045606 | −0.1208031 | −0.2128522 |
| ITGB3 | | | | | | | | | |
| VEGFA)- | 0.053245251 | 0.056273 | −0.0690103 | 0.10054105 | 0.05663497 | 0.07560646 | −0.0210205 | −0.1061318 | −0.2860545 |
| GRIN2B | | | | | | | | | |

TABLE 7B
Extended Data

| | Immune)- | Immune)- | Immune)- | CAF)- | CAF)- | CAF)- | Epithelial)- | Epithelial)- | Epithelial)- |
|---|---|---|---|---|---|---|---|---|---|
| | CAF | Epithelial | Immune | Epithelial | Immune | CAF | Immune | CAF | Epithelial |
| SEMA3F)- | −0.0100814 | −0.0652962 | 0.1318254 | −0.0115287 | −0.1082979 | −0.0835365 | −0.372915 | 0.31654534 | 0.14940711 |
| PLXNA3 | | | | | | | | | |
| SEMA3F)- | −0.1463495 | 0.14858009 | 0.16779253 | 0.01192398 | −0.2358 | 0.07293211 | 0.17019735 | 0.26082087 | 0.48458498 |
| PLXNA1 | | | | | | | | | |
| SEMA3F)- | −0.1205971 | 0.3372208 | 0.0028996 | 0.17345762 | 0.06318772 | −0.1172827 | 0.21808599 | −0.1816505 | 0.17747036 |
| NRP1 | | | | | | | | | |
| SEMA3F)- | −0.0502817 | 0.25630889 | 0.12184514 | 0.08900161 | 0.00909241 | −0.2262634 | −0.0773488 | −0.2136716 | 0.0541502 |
| NRP2 | | | | | | | | | |
| HEBP1)- | −0.0409831 | 0.06279032 | −0.1410404 | −0.0907204 | 0.3155658 | −0.042891 | 0.15271098 | −0.2408511 | 0.20461133 |
| ADRA2A | | | | | | | | | |
| HEBP1)- | 0.05850766 | −0.086905 | 0.12349588 | −0.0239813 | −0.0936212 | 0.0098824 | −0.1177402 | 0.16521739 | 0.06403162 |
| FPR3 | | | | | | | | | |
| DCN)- | 0.30817879 | −0.3214756 | 0.01377311 | 0.19130435 | −0.2605028 | −0.5712968 | 0.07011764 | 0.37781218 | −0.3967062 |
| EGFR | | | | | | | | | |
| DCN)- MET | 0.07773386 | 0.16666667 | 0.12002635 | 0.01884058 | −0.2362319 | −0.0679842 | 0.1400527 | 0.08932806 | 0.02529644 |
| GRN)- | 0.21746434 | 0.12555995 | 0.26620534 | −0.0280632 | −0.1304993 | −0.030304 | −0.1540287 | −0.0735202 | −0.1708827 |
| TNFRSF1A | | | | | | | | | |
| GRN)- | −0.4167463 | 0.06403162 | −0.4379716 | −0.3156785 | 0.18333389 | 0.18814849 | 0.17806189 | 0.31568892 | −0.1494071 |
| EGFR | | | | | | | | | |
| GRN)- | 0.09644269 | −0.0558648 | 0.29608644 | −0.2177279 | −0.0820925 | 0.3685112 | −0.1031756 | −0.0322793 | 0.09868573 |
| SORT1 | | | | | | | | | |
| GRN)- | −0.382226 | −0.3656126 | 0.37834103 | 0.29367589 | −0.1477112 | 0.21858427 | −0.1300465 | 0.25244573 | 0.23794466 |
| TNFRSF1B | | | | | | | | | |
| GRN)- | 0.19131695 | 0.00645586 | 0.11157244 | −0.0571805 | 0.18478979 | 0.112458 | −0.1741795 | −0.1373608 | 0.09354414 |
| CLEC4M | | | | | | | | | |
| ICAM3)- | −0.0406118 | 0.05485234 | 0.09908305 | 0.21357049 | −0.2214093 | 0.14716733 | −0.064531 | 0.07496706 | 0.08050066 |
| ITGAL | | | | | | | | | |
| ICAM3)- | 0.03968883 | −0.023866 | 0.10102544 | 0.11462451 | −0.1924027 | 0.08695652 | −0.2065694 | 0.23860343 | 0.21357049 |
| ITGB2 | | | | | | | | | |
| ICAM3)- | 0.13885413 | 0.1102321 | −0.1981269 | 0.11357049 | 0.0837617 | 0.09723961 | 0.18189009 | 0.16450359 | 0.23333333 |
| CLEC4M | | | | | | | | | |
| ICAM3)- | 0.01140747 | 0.09015991 | 0.05190095 | −0.0685793 | 0.16763864 | −0.0053368 | 0.19182235 | 0.16669412 | 0.18636978 |
| CD209 | | | | | | | | | |
| CEACAM1)- | 0.24724135 | −0.3501976 | 0.12712117 | −0.2822134 | 0.01607961 | 0.227939 | −0.0295232 | −0.0160743 | 0.31185771 |
| EGFR | | | | | | | | | |
| CEACAM1)- | −0.2356042 | −0.1633124 | 0.24107927 | −0.1185151 | 0.18527816 | −0.2104362 | −0.1843547 | 0.12570826 | 0.11653875 |
| SELE | | | | | | | | | |
| CEACAM1)- | −0.0281338 | 0.44586449 | −0.0543639 | 0.35356896 | −0.0828309 | 0.00401911 | −0.1920859 | −0.0796574 | −0.178135 |
| CD209 | | | | | | | | | |
| ST6GAL1)- | −0.057951 | −0.1593434 | 0.32744182 | 0.14808959 | −0.0906125 | −0.1324813 | −0.3182313 | −0.1154188 | 0.29947299 |
| EGFR | | | | | | | | | |
| NTN1)- | −0.3030742 | 0.07959675 | 0.08989356 | 0.31965215 | −0.0266869 | 0.15816696 | −0.2186347 | −0.1356658 | −0.0009882 |
| UNC5D | | | | | | | | | |
| NTN1)- | −0.0478387 | −0.0409831 | 0.00520524 | 0.21977008 | 0.06759116 | 0.06311559 | 0.06785467 | −0.0893369 | 0.09381073 |
| UNC5B | | | | | | | | | |
| NTN1)- | −0.1029257 | 0.15800224 | 0.27704372 | 0.07753879 | −0.2167754 | 0.17992555 | 0.22112407 | −0.1106829 | 0.1069864 |
| UNC5A | | | | | | | | | |
| NTN1)- | −0.0948804 | 0.18591856 | 0.21284883 | 0.08824983 | 0.04220664 | 0.32036628 | −0.1386224 | 0.14934616 | 0.18974207 |
| ADORA2B | | | | | | | | | |
| NTN1)- | −0.0425672 | 0.1180734 | 0.16277844 | −0.0743107 | 0.11896683 | −0.1887538 | 0.07096498 | 0.01383536 | 0.04881584 |
| NEO1 | | | | | | | | | |
| SCT)- | −0.2679013 | −0.1271254 | 0.17244907 | 0.13307421 | 0.0844625 | 0.31293234 | 0.0282851 | −0.0707533 | 0.08155468 |
| PTH1R | | | | | | | | | |
| SCT)- | 0.06807698 | 0.19961778 | −0.0153593 | 0.27148457 | 0.00382195 | 0.13017557 | 0.19932789 | −0.1152833 | 0.35243742 |
| RAMP2 | | | | | | | | | |
| SCT)- | 0.147494 | −0.1915307 | 0.21128804 | −0.0045461 | −0.1584498 | 0.15831082 | 0.08726315 | 0.16515696 | −0.023586 |
| VIPR1 | | | | | | | | | |
| SCT)- | −0.1306597 | 0.20040861 | 0.03998156 | 0.16607925 | 0.05617172 | 0.34641104 | 0.12032629 | −0.0549443 | 0.23175231 |
| ADRB2 | | | | | | | | | |
| SCT)- | 0.24876409 | 0.04692392 | −0.0104177 | 0.06390408 | −0.1847812 | 0.12420519 | −0.1098698 | 0.05547869 | −0.046576 |
| ADRB3 | | | | | | | | | |
| SCT)- | −0.1794517 | −0.2131936 | 0.40338157 | 0.43347936 | −0.0278071 | 0.32339669 | 0.03815109 | 0.014361 | 0.04729908 |
| GPR84 | | | | | | | | | |
| EFNB1)- | −0.2306349 | 0.05073968 | −0.0469126 | −0.0542855 | −0.0562953 | −0.0216747 | −0.1337882 | 0.1033699 | 0.54880258 |
| EPHB6 | | | | | | | | | |
| EFNB1)- | −0.0360438 | −0.0020428 | 0.00596533 | −0.1548192 | 0.2106165 | 0.00237162 | 0.3201529 | 0.35542526 | −0.2776295 |
| EPHB4 | | | | | | | | | |
| EFNB1)- | 0.11142594 | 0.11478651 | 0.32459147 | 0.02200336 | 0.05388847 | 0.012451 | −0.0034258 | 0.08432703 | 0.10659464 |
| ERBB2 | | | | | | | | | |
| EFNB1)- | −0.3118595 | −0.0620717 | 0.03295544 | −0.0852465 | 0.1696814 | −0.1498979 | 0.09304778 | 0.029717 | 0.18064431 |
| EPHB1 | | | | | | | | | |
| EFNB1)- | −0.243814 | −0.0690564 | 0.08222883 | 0.17431404 | 0.02333784 | −0.1007313 | −0.0982991 | 0.11733702 | −0.1919099 |
| EPHB3 | | | | | | | | | |
| EFNB1)- | −0.1042504 | 0.01383764 | 0.03407481 | 0.18643565 | −0.028466 | 0.04911553 | 0.08276498 | 0.15730004 | −0.1384149 |
| EPHB2 | | | | | | | | | |
| EFNB1)- | 0.17833366 | −0.0647074 | 0.01614764 | 0.06232089 | −0.0217449 | −0.1602689 | 0.35952687 | −0.1420852 | −0.0813624 |
| EPHA4 | | | | | | | | | |
| DLL3)- | −0.3398379 | 0.03052178 | 0.47965579 | −0.1446029 | −0.1054435 | −0.1920416 | −0.0241532 | −0.0283945 | 0.06034454 |
| NOTCH1 | | | | | | | | | |
| DLL3)- | −0.0168815 | 0.33672838 | −0.0001978 | 0.16522283 | −0.0492554 | −0.1794458 | −0.2082235 | 0.11196416 | 0.08972628 |
| NOTCH4 | | | | | | | | | |
| DLL3)- | −0.143248 | −0.1990178 | 0.14993079 | −0.1281992 | −0.1033801 | −0.0897263 | −0.1701258 | −0.0511216 | −0.0318851 |
| NOTCH2 | | | | | | | | | |
| DLL3)- | −0.1087709 | 0.02129273 | 0.02528183 | 0.14855562 | 0.11542262 | −0.1834053 | 0.12675407 | −0.1921671 | 0.07991041 |
| NOTCH3 | | | | | | | | | |
| SEMA4G)- | 0.06640535 | −0.1832735 | −0.0598175 | −0.3 | 0.02832675 | 0.04150198 | 0.13056653 | 0.05243742 | 0.24216074 |
| PLXNB2 | | | | | | | | | |
| VCL)- | −0.3532358 | 0.1989522 | 0.16356388 | −0.0249012 | 0.13662714 | 0.2859778 | 0.07865613 | −0.2024441 | 0.20474308 |
| ITGB5 | | | | | | | | | |
| EFNA2)- | −0.0504977 | 0.05477557 | 0.11014686 | −0.1981222 | −0.1958574 | 0.21666502 | −0.0613396 | 0.15851891 | 0.0198946 |
| EPHA3 | | | | | | | | | |
| EFNA2)- | 0.06665129 | −0.0711225 | 0.27661681 | 0.17908088 | −0.19459 | 0.1537397 | −0.2078008 | 0.1191237 | 0.06324111 |
| EPHA1 | | | | | | | | | |
| EFNA2)- | 0.35160887 | 0.26141986 | −0.0704137 | −0.2026684 | −0.0771715 | −0.1445378 | 0.04335651 | −0.181944 | 0.04545455 |
| EPHA4 | | | | | | | | | |
| EFNA2)- | 0.12099142 | −0.1678861 | 0.03104608 | 0.15483446 | −0.2765936 | −0.2072215 | −0.08722 | −0.1967127 | 0.28511199 |
| EPHA2 | | | | | | | | | |
| MADCAM1)- | −0.0316372 | −0.0831136 | 0.10906693 | −0.0395942 | 0.03951358 | −0.0422294 | 0.09912345 | −0.2002635 | −0.0642951 |
| ITGA4 | | | | | | | | | |
| MADCAM1)- | 0.25237283 | −0.2109808 | 0.21117852 | −0.1081099 | 0.08320706 | 0.08156005 | −0.0379447 | −0.2770751 | −0.0289855 |
| CD44 | | | | | | | | | |
| MADCAM1)- | −0.1866658 | −0.0213558 | 0.21984181 | 0.03261192 | 0.08364631 | −0.1859868 | 0.0994861 | 0.02556079 | −0.1154847 |
| ITGB7 | | | | | | | | | |
| MIF)- | 0.10996541 | 0.04579147 | −0.111748 | −0.3561499 | 0.10323813 | −0.2815073 | −0.0021081 | 0.2198946 | 0.22806324 |
| CXCR4 | | | | | | | | | |
| MIF)- CD44 | 0.43419536 | 0.15760172 | −0.0493494 | 0.13953488 | 0.10290533 | 0.21661506 | −0.1032938 | 0.10500659 | −0.0810277 |
| MIF)- | 0.11128682 | −0.1915994 | 0.01799367 | 0.07694842 | −0.118529 | −0.1640149 | 0.12665987 | 0.20982246 | −0.1179183 |
| EGFR | | | | | | | | | |
| MIF)- | 0.17678648 | 0.14147804 | −0.0022428 | −0.1979311 | 0.15328814 | 0.11322309 | −0.1165414 | 0.10389354 | −0.0491501 |
| CXCR2 | | | | | | | | | |
| OSM)- | −0.0663282 | −0.0085712 | 0.2346461 | 0.11002042 | −0.1417178 | 0.23044996 | −0.2336289 | 0.07035805 | 0.04782766 |
| LIFR | | | | | | | | | |
| OSM)- | −0.0026373 | −0.059735 | 0.14581964 | 0.1067264 | −0.1123336 | 0.13755847 | 0.08116744 | 0.17879377 | 0.18854376 |
| IL6ST | | | | | | | | | |
| OSM)- | 0.12370593 | 0.01747215 | −0.0639238 | −0.0328744 | −0.2155681 | 0.10430256 | 0.05448317 | −0.1267007 | 0.08551006 |
| OSMR | | | | | | | | | |
| LGALS1)- | −0.2270092 | 0.01752306 | 0.14216074 | −0.1951252 | −0.2009223 | 0.8083004 | 0.36996047 | −0.2342556 | 0.23517787 |
| ITGB1 | | | | | | | | | |
| LGALS1)- | 0.24110672 | 0.25915679 | −0.1487266 | −0.4617918 | 0.32644725 | −0.3345191 | −0.1971599 | 0.3372859 | 0.35296443 |
| PTPRC | | | | | | | | | |
| NID2)- | 0.01060711 | 0.11271781 | 0.05298537 | −0.0993412 | 0.09173285 | −0.047434 | 0.16804509 | −0.0429541 | 0.16376812 |
| COL13A1 | | | | | | | | | |
| GZMB)- | −0.2718108 | 0.16506326 | 0.13779242 | 0.04953887 | −0.0268134 | 0.22635046 | −0.1579155 | 0.03623188 | −0.086166 |
| CHRM3 | | | | | | | | | |
| GZMB)- | 0.27617793 | −0.1037823 | 0.20255709 | −0.1067194 | −0.1081206 | 0.20007906 | −0.0888816 | −0.0407141 | 0.08366271 |
| IGF2R | | | | | | | | | |
| OXT)- | 0.21514024 | 0.1927044 | 0.23936418 | 0.28888596 | −0.2713547 | 0.47282072 | 0.03804188 | 0.48174991 | 0.40355731 |
| AVPR1B | | | | | | | | | |
| OXT)- | 0.251071 | 0.14459421 | −0.1775097 | 0.17043284 | −0.0256976 | −0.0253648 | −0.0341954 | 0.05316381 | 0.13807642 |
| OXTR | | | | | | | | | |
| F9)- LRP1 | 0.05339839 | −0.0468045 | 0.01628268 | −0.2040843 | −0.1886693 | −0.0803716 | 0.03372859 | 0.16423466 | 0.03491436 |
| PCSK1N)- | 0.29058313 | 0.20197763 | −0.1263269 | −0.0259552 | −0.0538315 | −0.0988834 | 0.16716084 | 0.12404888 | 0.05243742 |
| GPR171 | | | | | | | | | |
| TIMP1)- | 0.14302655 | 0.26482213 | −0.0370713 | −0.1960474 | −0.2275068 | 0.01633836 | 0.01741426 | 0.04690691 | 0.42239789 |
| FGFR2 | | | | | | | | | |
| TIMP1)- | −0.4495389 | 0.41608749 | −0.0066537 | −0.0480912 | 0.30310616 | 0.27312253 | −0.19164 | −0.5231884 | 0.5412563 |
| CD63 | | | | | | | | | |
| MMP2)- | 0.29683794 | 0.14980237 | −0.2569395 | −0.0082348 | 0.08518778 | −0.0759577 | −0.2061714 | 0.29841897 | 0.08906456 |
| PECAM1 | | | | | | | | | |
| MMP2)- | −0.1554677 | 0.19262187 | 0.16179183 | −0.0526368 | −0.0355743 | 0.2677295 | 0.20487484 | −0.1222661 | 0.19288538 |
| SDC2 | | | | | | | | | |
| MMP2)- | −0.1623954 | 0.27088274 | 0.44195547 | −0.1248394 | −0.0830835 | 0.29950259 | 0.46356569 | −0.3116806 | 0.35059289 |
| FGFR1 | | | | | | | | | |
| CCL22)- | −0.1383901 | 0.23539492 | 0.24924942 | −0.0280632 | −0.0364189 | 0.22555995 | 0.09903027 | 0.04967062 | 0.06745718 |
| CCR4 | | | | | | | | | |
| CCL22)- | 0.05453407 | 0.29035555 | 0.1572856 | −0.2144928 | −0.3662734 | 0.21384104 | −0.1351441 | 0.10461478 | −0.4353096 |
| DPP4 | | | | | | | | | |
| CCL17)- | −0.0032945 | 0.00019767 | 0.16497309 | 0.10421951 | −0.1356845 | 0.12404888 | −0.2592205 | 0.36179183 | 0.25744401 |
| CCR4 | | | | | | | | | |
| TG)- | −0.0779324 | −0.0637569 | 0.27119763 | 0.45826884 | 0.01652102 | 0.12237636 | −0.1483387 | 0.03201581 | 0.16073781 |
| ASGR1 | | | | | | | | | |
| SFRP1)- | −0.1293237 | 0.16118935 | 0.01094518 | −0.1741765 | −0.0807748 | 0.19940693 | −0.1005403 | −0.1267875 | 0.50685112 |
| FZD6 | | | | | | | | | |
| PLAT)- | 0.16798419 | 0.12582345 | −0.18902 | 0.28827404 | −0.2505767 | 0.16350461 | 0.06623608 | 0.05638999 | −0.183531 |
| ITGAM | | | | | | | | | |
| PLAT)- | −0.3054119 | 0.16508564 | 0.11725955 | −0.1918314 | −0.1610013 | −0.0695016 | 0.08418972 | 0.13307421 | 0.2088274 |
| LRP1 | | | | | | | | | |
| PLAT)- | 0.2599473 | 0.20184453 | −0.279577 | 0.19235837 | −0.2843212 | 0.22806324 | 0.28590255 | −0.2791831 | −0.2326746 |
| ITGB2 | | | | | | | | | |
| DKK4)- | 0.00276899 | −0.1347403 | 0.15888562 | −0.2255099 | 0.01067405 | 0.08537549 | 0.01594518 | −0.0760211 | 0.03327952 |
| LRP6 | | | | | | | | | |
| DKK4)- | −0.1018726 | 0.14992587 | 0.19310396 | 0.02378207 | 0.13017128 | 0.10805113 | −0.1919631 | 0.06746607 | −0.011924 |
| LRP5 | | | | | | | | | |
| LHB)- | 0.15798687 | −0.1726914 | −0.2928303 | 0.17168456 | −0.1453911 | 0.25470238 | 0.24170906 | 0.10349485 | 0.35296443 |
| PTH1R | | | | | | | | | |
| LHB)- | −0.1322561 | −0.1527046 | 0.12440625 | 0.07740958 | −0.0735091 | 0.09730549 | 0.0233922 | 0.09828722 | 0.15046113 |
| RAMP2 | | | | | | | | | |
| LHB)- | −0.0263202 | −0.1316424 | 0.09509985 | −0.0430901 | −0.2527766 | 0.07161445 | −0.1474378 | 0.10342897 | −0.0281978 |
| VIPR1 | | | | | | | | | |
| LHB)- | −0.1056799 | −0.1748023 | 0.04943658 | −0.2183938 | 0.09693406 | 0.14366188 | 0.24553692 | 0.02918506 | 0.14637681 |
| ADRB2 | | | | | | | | | |
| LHB)- | 0.08537315 | −0.2038657 | −0.215971 | 0.23520111 | −0.0312428 | 0.13040327 | 0.09451311 | 0.19938065 | 0.09242729 |
| ADRB3 | | | | | | | | | |
| LHB)- | 0.22925204 | −0.0757916 | −0.2933426 | 0.01166085 | 0.08693938 | 0.05791658 | −0.1132512 | −0.0777441 | 0.1969697 |
| LHCGR | | | | | | | | | |
| LHB)- | −0.051847 | −0.1009236 | −0.0071916 | 0.28526253 | −0.0664558 | 0.17240925 | −0.0135736 | 0.35652174 | 0.31963109 |
| GPR84 | | | | | | | | | |
| AMH)- | 0.10082067 | 0.03545538 | 0.07564915 | 0.06154049 | 0.12808006 | 0.03819159 | −0.0981926 | 0.02431791 | 0.17914676 |
| ACVR1 | | | | | | | | | |
| AMH)- | −0.0308392 | −0.0378888 | −0.022346 | −0.1282034 | −0.1855867 | −0.308232 | 0.0642876 | 0.01311022 | −0.1287921 |
| EGFR | | | | | | | | | |
| EBI3)- | −0.0699024 | −0.0347419 | 0.09805345 | 0.20046774 | −0.0110118 | −0.1566444 | −0.2575499 | −0.1678419 | 0.13913043 |
| IL27RA | | | | | | | | | |
| EBI3)- | 0.02775398 | 0.11661945 | 0.03144779 | 0.3737936 | 0.24844352 | 0.27741362 | 0.22010673 | 0.23952569 | 0.36455863 |
| IL6ST | | | | | | | | | |
| TGFB1)- | 0.06327237 | 0.07130384 | 0.05236086 | −0.232814 | −0.0478608 | −0.1908088 | −0.0501665 | −0.3365397 | −0.2534914 |
| ENG | | | | | | | | | |
| TGFB1)- | −0.2269013 | 0.03308182 | −0.0376426 | 0.13228367 | −0.0639581 | −0.006061 | −0.2562852 | −0.016799 | 0.30803689 |
| EGFR | | | | | | | | | |
| TGFB1)- | −0.1598076 | 0.07163334 | −0.0905167 | 0.04367733 | 0.00250346 | 0.11601173 | −0.0301064 | 0.06113307 | −0.2 |
| SMAD3 | | | | | | | | | |
| TGFB1)- | 0.10735469 | −0.1422123 | 0.13300379 | 0.08247966 | −0.0803821 | −0.1384149 | −0.0731322 | −0.4303831 | −0.1139657 |
| TGFBR2 | | | | | | | | | |
| TGFB1)- | −0.1450699 | 0.10683099 | 0.15158006 | −0.0860757 | −0.1577231 | 0.16953285 | −0.0111997 | 0.14495141 | 0.01324198 |
| ITGB3 | | | | | | | | | |
| TGFB1)- | −0.136831 | 0.09937725 | −0.0883923 | −0.0306993 | 0.12730628 | 0.18557686 | −0.064903 | 0.20668753 | 0.09077734 |
| ACVRL1 | | | | | | | | | |
| TGFB1)- | −0.0347952 | 0.01067581 | −0.03954 | 0.18353701 | −0.1453276 | 0.04209625 | 0.043083 | 0.05296443 | 0.07786561 |
| ITGB6 | | | | | | | | | |
| TGFB1)- | 0.10056345 | −0.1600712 | 0.07663404 | −0.0470371 | −0.0920623 | 0.21792549 | −0.0287315 | 0.02345191 | 0.04295125 |
| ITGAV | | | | | | | | | |
| TGFB1)- | 0.07723484 | −0.0752578 | −0.0187815 | −0.012451 | −0.0672618 | 0.11265193 | 0.09578393 | −0.0496706 | 0.22384717 |
| ITGB1 | | | | | | | | | |
| TGFB1)- | 0.24066692 | −0.2852153 | −0.3349809 | 0.26390856 | 0.28134264 | −0.0176554 | 0.10744754 | −0.1312253 | 0.36772069 |
| CAV1 | | | | | | | | | |
| TGFB1)- | 0.03914462 | 0.17937989 | 0.27025604 | −0.2577819 | −0.2553444 | 0.20843901 | −0.0388669 | −0.0795784 | 0.08959157 |
| SDC2 | | | | | | | | | |
| TGFB1)- | −0.1460345 | −0.0301513 | −0.0481416 | 0.04045196 | 0.02253113 | 0.16304885 | −0.1573833 | 0.08432148 | 0.28440609 |
| ITGB8 | | | | | | | | | |
| TGFB1)- | −0.1921645 | −0.192494 | 0.21503889 | −0.0175895 | 0.02147704 | 0.00039527 | −0.0083665 | −0.0094862 | −0.0880105 |
| CXCR4 | | | | | | | | | |
| TGFB1)- | 0.18999606 | 0.03940822 | 0.08553824 | 0.09776343 | 0.11212491 | 0.09697609 | 0.07812912 | −0.0380777 | 0.21317523 |
| ITGB5 | | | | | | | | | |
| ICAM5)- | −0.416241 | −0.4486703 | 0.47884584 | 0.24342842 | −0.2723797 | 0.31569932 | −0.2530486 | 0.15098814 | 0.31818182 |
| ITGAL | | | | | | | | | |
| ICAM5)- | −0.0673632 | −0.1020334 | −0.0433478 | 0.14612293 | −0.2138974 | 0.18769352 | −0.1274339 | 0.11936759 | 0.09235837 |
| ITGB2 | | | | | | | | | |
| CEACAM5)- | −0.034919 | 0.25112817 | 0.14947791 | 0.21930894 | 0.12984617 | −0.1250494 | −0.2726704 | 0.15430228 | −0.167858 |
| CD1D | | | | | | | | | |
| COMP)- | −0.2043478 | 0.02424242 | 0.13201581 | 0.16613966 | 0.06192358 | 0.07667984 | −0.054809 | −0.0909091 | −0.1628458 |
| ITGB1 | | | | | | | | | |
| COMP)- | 0.04276067 | 0.0266816 | 0.0128467 | −0.0188418 | 0.02206997 | 0.13520013 | 0.09506555 | 0.02740899 | 0.10481586 |
| ITGB3 | | | | | | | | | |
| COMP)- | −0.0910438 | 0.00823479 | 0.08360259 | 0.12391713 | 0.01298873 | 0.06100333 | 0.03804313 | −0.0442044 | −0.0225963 |
| CD36 | | | | | | | | | |
| HGF)- | 0.09860753 | −0.1160982 | 0.09722148 | 0.00685157 | −0.0216747 | −0.3058831 | 0.03965744 | −0.1393939 | −0.1996047 |
| CD44 | | | | | | | | | |
| HGF)- | 0.02158277 | 0.08283297 | 0.10243567 | −0.0471046 | −0.0918374 | −0.1150273 | −0.1732543 | −0.3698287 | 0.22279315 |
| SDC1 | | | | | | | | | |
| HGF)- | 0.07128255 | −0.0797309 | −0.0112204 | 0.08281178 | 0.00942091 | −0.257988 | 0.00263505 | −0.286166 | 0.11277997 |
| ITGB1 | | | | | | | | | |
| HGF)- MET | −0.0737906 | 0.00600622 | 0.13147671 | −0.1787997 | 0.17438566 | 0.21898676 | 0.18853755 | 0.28366271 | −0.3225296 |
| HGF)- | −0.0872551 | 0.18632468 | 0.20341929 | 0.04717043 | −0.0630476 | −0.1803149 | 0.21949934 | −0.5520422 | 0.31488801 |
| SDC2 | | | | | | | | | |
| HGF)- | −0.1425226 | 0.10540578 | 0.34927604 | 0.16753409 | −0.0751161 | 0.18950351 | 0.06707297 | 0.14053698 | 0.23952569 |
| NRP1 | | | | | | | | | |
| HGF)- ST14 | −0.0506898 | 0.1868527 | −0.084021 | −0.3799987 | 0.1553462 | 0.06943804 | 0.08787879 | 0.00263505 | −0.3550725 |
| LAMB1)- | 0.15719086 | −0.0069829 | −0.0001978 | 0.1200303 | −0.0334949 | 0.07965214 | 0.11531619 | −0.0083668 | −0.3339921 |
| ITGA7 | | | | | | | | | |
| LAMB1)- | −0.2928854 | −0.0537549 | 0.20771006 | −0.1654205 | −0.0586181 | 0.57149445 | 0.24329491 | −0.2837945 | −0.1363636 |
| ITGAV | | | | | | | | | |
| LAMB1)- | −0.4483531 | 0.12160738 | 0.25928854 | −0.1617313 | −0.149412 | 0.56635594 | 0.16113307 | −0.5603426 | 0.03926219 |
| ITGB1 | | | | | | | | | |
| LAMB1)- | 0.39525692 | −0.0393939 | 0.31910408 | −0.1097533 | −0.183537 | −0.178135 | 0.24216074 | 0.30500659 | 0.01528327 |
| ITGA6 | | | | | | | | | |
| LAMB1)- | −0.1166085 | 0.15533597 | 0.08532367 | −0.0310287 | 0.08295447 | 0.28059426 | −0.0540273 | −0.0375519 | 0.00474308 |
| ITGA1 | | | | | | | | | |
| LAMB1)- | 0.22160738 | −0.1853999 | 0.39670619 | 0.25900181 | −0.2581771 | −0.1021114 | 0.44150198 | 0.26982872 | −0.3511662 |
| ITGA2 | | | | | | | | | |
| NAMPT)- | 0.0293827 | 0.00777339 | −0.120273 | 0.23465086 | 0.03125517 | −0.0303709 | 0.08334985 | 0.06891327 | −0.0724003 |
| ADORA2A | | | | | | | | | |
| PON2)- | 0.17140975 | 0.1541502 | 0.11317523 | 0.27009223 | 0.00711462 | 0.23847167 | −0.1737813 | −0.0438735 | −0.1816864 |
| HTR2A | | | | | | | | | |
| PDAP1)- | −0.120559 | −0.2459298 | −0.3695209 | −0.0252314 | −0.1298462 | 0.01205573 | −0.0651558 | −0.1592332 | 0.09882074 |
| PDGFRB | | | | | | | | | |
| C5)- | −0.274846 | 0.07831764 | −0.0967827 | 0.07464014 | −0.1182594 | −0.0660781 | 0.04170235 | −0.1701637 | 0.20764163 |
| ADRA2A | | | | | | | | | |
| C5)- C5AR2 | −0.1750066 | 0.17107263 | 0.0707174 | −0.0586976 | 0.11063157 | 0.21932339 | 0.21097714 | −0.1897358 | 0.01963109 |
| C5)- C5AR1 | −0.0214253 | −0.0323027 | −0.0097606 | −0.1530353 | 0.18130294 | −0.1004644 | −0.037037 | 0.15428195 | 0.08511199 |
| TNFSF8)- | −0.2255045 | 0.03896748 | 0.04087158 | 0.04795784 | −0.1377446 | −0.0035574 | −0.2835376 | −0.0318851 | 0.20421607 |
| TNFRSF8 | | | | | | | | | |
| CSF3)- | −0.1158714 | −0.0834432 | −0.0262533 | 0.41805007 | −0.071226 | 0.41383399 | 0.21533529 | −0.0770118 | −0.0306993 |
| CSF1R | | | | | | | | | |
| CSF3)- | −0.0145004 | 0.10334829 | 0.08774938 | 0.11831357 | 0.04264857 | 0.20105402 | 0.0359921 | 0.22266873 | 0.12536645 |
| CSF3R | | | | | | | | | |
| WNT3)- | −0.2313174 | 0.21205042 | 0.15533799 | 0.042228 | 0.03603782 | −0.2995915 | 0.05448317 | −0.0290523 | 0.1801054 |
| RYK | | | | | | | | | |
| WNT3)- | −0.0080467 | −0.013394 | 0.16650733 | 0.13259524 | −0.0405891 | 0.05612833 | 0.15721157 | −0.1162055 | 0.22056741 |
| LRP6 | | | | | | | | | |
| WNT3)- | 0.14320122 | −0.1160176 | −0.2294348 | 0.09651174 | −0.1487778 | 0.41855026 | 0.06417394 | 0.28770058 | 0.24018445 |
| ROR2 | | | | | | | | | |
| WNT3)- | −0.0476756 | 0.11351128 | −0.0989707 | 0.07332257 | −0.1349677 | −0.11768 | −0.0046789 | −0.0955645 | 0.30777339 |
| FZD1 | | | | | | | | | |
| WNT3)- | 0.1600819 | 0.09000072 | 0.1230912 | 0.15455564 | 0.09131939 | −0.0754002 | −0.0362367 | −0.1534306 | 0.16990019 |
| FZD7 | | | | | | | | | |
| WNT3)- | −0.0904924 | 0.24516062 | 0.14485063 | 0.13122962 | −0.0885338 | 0.22728021 | −0.3049653 | 0.16231884 | 0.05862978 |
| FZD8 | | | | | | | | | |
| WNT3)- | −0.0860187 | −0.0615395 | −0.205574 | 0.07582845 | 0.1924496 | 0.07511118 | 0.1295912 | 0.25405192 | −0.2029052 |
| FZD5 | | | | | | | | | |
| CCL2)- | −0.1099908 | −0.1854488 | 0.01768861 | 0.22229543 | 0.18733109 | 0.07010146 | −0.0544981 | 0.21100827 | 0.15079548 |
| CCR4 | | | | | | | | | |
| CCL2)- | −0.185976 | −0.5548966 | 0.31069051 | 0.39860324 | −0.0907892 | 0.29476875 | 0.03447936 | −0.2327481 | 0.00335979 |
| CCR1 | | | | | | | | | |
| CCL2)- | −0.0651773 | −0.0711085 | 0.05300982 | 0.08077481 | 0.00355942 | 0.115562 | −0.1066403 | 0.03537666 | −0.101255 |
| CCR5 | | | | | | | | | |
| CCL2)- | −0.3042046 | −0.1635908 | 0.2111232 | 0.26489193 | −0.2748097 | 0.23929372 | 0.12414317 | 0.05626009 | 0.12465821 |
| CCR3 | | | | | | | | | |
| CCL13)- | −0.2144104 | −0.0873834 | 0.01727207 | −0.0889504 | −0.0695383 | 0.29909744 | −0.1094744 | 0.0256917 | 0.1125313 |
| CCR3 | | | | | | | | | |
| CCL13)- | −0.0189767 | 0.09501532 | 0.07104943 | −0.1296528 | −0.0306567 | 0.12569998 | 0.18030198 | −0.1507246 | −0.0690382 |
| CCR1 | | | | | | | | | |
| CCL13)- | −0.1662439 | −0.1836392 | 0.26662053 | 0.08452467 | −0.0127208 | 0.11311681 | 0.09444409 | −0.197892 | −0.0478261 |
| CCR5 | | | | | | | | | |
| COL1A1)- | −0.2954643 | 0.40632411 | 0.48203169 | −0.4345191 | −0.3201028 | 0.78098752 | 0.45304523 | −0.1681874 | 0.37641634 |
| ITGA5 | | | | | | | | | |
| COL1A1)- | 0.34729908 | −0.0567852 | −0.1511536 | −0.2570487 | 0.24976934 | −0.0163373 | −0.0815425 | 0.3284585 | −0.0708827 |
| CD93 | | | | | | | | | |
| COL1A1)- | 0.01264989 | 0.07140975 | −0.1440949 | 0.14018445 | −0.1315105 | 0.20081697 | −0.1442925 | 0.13058374 | 0.09130435 |
| FLT4 | | | | | | | | | |
| COL1A1)- | 0.00349155 | 0.07378372 | −0.2357751 | −0.1490826 | −0.1043714 | 0.28004875 | −0.213358 | −0.0036892 | 0.0282618 |
| CD36 | | | | | | | | | |
| COL1A1)- | −0.1618684 | 0.22450593 | 0.13730852 | 0.09367589 | 0.1809916 | −0.0089597 | 0.25774996 | −0.1900652 | 0.31475626 |
| ITGA1 | | | | | | | | | |
| COL1A1)- | −0.3413156 | 0.47180501 | 0.48842037 | −0.1014493 | −0.1062099 | 0.29276327 | 0.45850767 | −0.2825521 | 0.41581028 |
| DDR2 | | | | | | | | | |
| COL1A1)- | 0.17167325 | 0.17931489 | −0.0920949 | −0.2227931 | 0.514361 | 0.46943347 | 0.0226614 | 0.20803689 | 0.07747036 |
| CD44 | | | | | | | | | |
| COL1A1)- | −0.1951252 | 0.13109354 | 0.16685339 | 0.24729908 | −0.0299176 | 0.58787879 | 0.18583197 | −0.0457181 | 0.17312253 |
| ITGAV | | | | | | | | | |
| COL1A1)- | −0.2841897 | 0.0969697 | 0.26113307 | −0.1397892 | −0.1476943 | 0.83465086 | 0.25467721 | −0.2075099 | 0.10922266 |
| ITGB1 | | | | | | | | | |
| COL1A1)- | −0.4300537 | 0.58511199 | 0.56681709 | −0.3779974 | −0.2689812 | 0.48163642 | 0.55403669 | −0.3647024 | 0.58814229 |
| ITGA11 | | | | | | | | | |
| COL1A1)- | 0.23504611 | −0.1628673 | 0.20250329 | 0.59098696 | −0.4638999 | −0.4980237 | 0.1140975 | 0.16337286 | −0.1095006 |
| ITGA2 | | | | | | | | | |
| COL1A1)- | 0.23913043 | −0.1638999 | 0.05125165 | 0.00566535 | −0.101581 | −0.2226614 | 0.08432148 | 0.29156785 | −0.2528327 |
| DDR1 | | | | | | | | | |
| VTN)- | −0.0899351 | 0.26326904 | 0.27362947 | −0.1252512 | −0.054095 | −0.1433748 | 0.01007938 | −0.1575151 | 0.20500659 |
| ITGA5 | | | | | | | | | |
| VTN)- | −0.033496 | 0.06257214 | 0.20912811 | 0.03584371 | −0.0904924 | 0.20235232 | 0.36085486 | −0.1100204 | 0.11726341 |
| ITGA8 | | | | | | | | | |
| VTN)- | −0.0885475 | 0.03764754 | −0.2274751 | −0.1263054 | 0.14693286 | 0.10739582 | −0.0073784 | 0.15428195 | −0.1839262 |
| CD47 | | | | | | | | | |
| VTN)- | 0.02874663 | 0.02736205 | 0.31400875 | 0.06384451 | −0.1331356 | −0.2422006 | −0.0461944 | −0.0457181 | −0.2824769 |
| ITGAV | | | | | | | | | |
| VTN)- | −0.0397574 | 0.1018 | 0.19667707 | 0.02978093 | −0.0150222 | −0.1777631 | 0.21870883 | −0.2361001 | 0.12977602 |
| ITGB1 | | | | | | | | | |
| VTN)- KDR | −0.2550934 | −0.0449001 | 0.07998022 | 0.11148081 | −0.2754688 | 0.35091419 | −0.1188954 | −0.0673254 | 0.14163373 |
| VTN)- | −0.0754929 | 0.26181852 | 0.11158376 | −0.1366496 | −0.1860419 | −0.0850601 | 0.15794156 | 0.06403162 | −0.0422925 |
| PLAUR | | | | | | | | | |
| VTN)- | −0.1212901 | −0.1577109 | 0.15542371 | 0.08914512 | −0.0578564 | 0.18157852 | 0.01278166 | −0.3009555 | −0.3123847 |
| ITGA2B | | | | | | | | | |
| VTN)- | −0.040885 | −0.0966636 | 0.11568644 | 0.07267815 | −0.2432708 | 0.22019769 | 0.05092562 | −0.3917641 | −0.1017195 |
| ITGB3 | | | | | | | | | |
| VTN)- | −0.0551856 | 0.10272305 | −0.0046812 | −0.198847 | 0.15826059 | 0.20820293 | 0.04677207 | 0.0944664 | −0.0226614 |
| ITGB6 | | | | | | | | | |
| VTN)- | −0.0046814 | −0.2030725 | −0.0062638 | 0.22922089 | −0.1903538 | −0.1441655 | 0.00665371 | 0.26390856 | −0.2413702 |
| TNFRSF11B | | | | | | | | | |
| VTN)- PVR | −0.1420236 | −0.0970528 | 0.13666791 | 0.15595454 | 0.08250412 | 0.08710549 | −0.1333553 | −0.1243124 | −0.215942 |
| VTN)- | −0.0619787 | 0.25496152 | 0.16476567 | −0.2451655 | −0.1960797 | −0.0760361 | 0.23030303 | −0.0883428 | 0.21291173 |
| ITGB5 | | | | | | | | | |
| BSP)- | −0.0098863 | −0.199453 | 0.22505602 | 0.19080248 | −0.0461194 | −0.1189998 | −0.095395 | 0.10205897 | 0.35614994 |
| ITGB3 | | | | | | | | | |
| ISP)- | 0.1049094 | −0.138056 | −0.2226763 | 0.13966665 | 0.20143008 | −0.2438896 | −0.0560132 | 0.04677207 | −0.0125165 |
| ITGAV | | | | | | | | | |
| CXCL6)- | 0.0727237 | −0.230361 | −0.0220882 | 0.09776343 | 0.19580327 | 0.16035312 | 0.31365703 | 0.23749135 | −0.1395257 |
| ADRA2A | | | | | | | | | |
| CXCL6)- | 0.18732079 | 0.03778314 | −0.2221196 | 0.08894746 | 0.02892196 | 0.0965181 | 0.19067419 | −0.0451281 | 0.27408091 |
| CXCR2 | | | | | | | | | |
| CXCL6)- | −0.0820337 | 0.0850503 | 0.04416391 | 0.02753714 | 0.37400636 | −0.2309492 | −0.0782801 | 0.10502735 | 0.34163373 |
| CXCR1 | | | | | | | | | |
| IL2)- | −0.0020447 | −0.0073212 | −0.1264554 | 0.16595296 | 0.0398959 | 0.04512814 | 0.44577679 | −0.2094862 | −0.172859 |
| IL2RA | | | | | | | | | |
| IL2)- | 0.30928061 | 0.23097996 | −0.3038502 | 0.11206272 | −0.0440256 | 0.04552492 | 0.29372613 | −0.2063968 | −0.2902503 |
| IL2RG | | | | | | | | | |
| IL2)- CD53 | −0.117007 | −0.0918116 | −0.1091503 | 0.09941366 | 0.01627623 | −0.0185124 | 0.23562746 | 0.00737813 | 0.16600791 |
| APOC3)- | 0.15427198 | −0.0143003 | 0.09720255 | 0.17272727 | −0.2284585 | −0.2040843 | 0.13293808 | 0.03280632 | 0.04057971 |
| LDLR | | | | | | | | | |
| APOC3)- | −0.239258 | 0.1971729 | 0.11723616 | −0.4192358 | −0.2617918 | 0.31173622 | −0.0669302 | 0.08603709 | −0.0512516 |
| LRP1 | | | | | | | | | |
| APOC3)- | −0.0992455 | 0.21694291 | 0.24811363 | −0.2110672 | −0.2320158 | 0.08985507 | −0.1205534 | −0.0368906 | −0.1440053 |
| SDC2 | | | | | | | | | |
| APOC3)- | −0.0902171 | −0.1018815 | 0.21124414 | −0.0977602 | 0.12210034 | −0.0364954 | 0.17041247 | −0.0935441 | −0.1027668 |
| TLR2 | | | | | | | | | |
| KITLG)- | −0.0757676 | −0.0124514 | −0.0445125 | −0.13426 | −0.0103453 | 0.06341206 | 0.17909858 | −0.2662318 | −0.0976317 |
| KIT | | | | | | | | | |
| SELPLG)- | −0.3070025 | −0.0176127 | 0.32133324 | 0.23386034 | −0.191854 | 0.13833992 | −0.2456748 | 0.11436477 | 0.21779374 |
| ITGAM | | | | | | | | | |
| SELPLG)- | 0.11752879 | 0.05481716 | −0.0284432 | −0.1052701 | −0.0686065 | 0.20548803 | −0.0355236 | −0.1662604 | 0.23518561 |
| ESAM | | | | | | | | | |
| SELPLG)- | −0.0112816 | 0.03885356 | 0.26316668 | 0.2284585 | −0.0926677 | 0.04098037 | −0.0740509 | 0.09533849 | −0.0567871 |
| SELL | | | | | | | | | |
| SELPLG)- | 0.17396195 | −0.3064189 | 0.02632449 | 0.14980731 | −0.1109537 | 0.04420581 | 0.0182513 | −0.078598 | 0.36188155 |
| SELP | | | | | | | | | |
| SELPLG)- | −0.2793339 | 0.02295668 | 0.00227866 | 0.09163675 | 0.01121299 | 0.35683226 | 0.02948454 | 0.01917312 | 0.16173661 |
| SELE | | | | | | | | | |
| SELPLG)- | −0.1487517 | −0.2269206 | 0.13133421 | 0.09894598 | −0.0604883 | 0.09617918 | −0.3386927 | 0.25481735 | 0.26516025 |
| ITGB2 | | | | | | | | | |
| IL23A)- | −0.3331137 | −0.3761411 | 0.05595859 | −0.1174843 | −0.0228127 | 0.33457421 | −0.0378403 | 0.21324813 | 0.13433475 |
| IL12RB2 | | | | | | | | | |
| IFNG)- | −0.1039133 | −0.1221073 | 0.18349054 | −0.0934155 | −0.1498732 | 0.12201067 | 0.27562582 | 0.19816199 | −0.470751 |
| IFNGR2 | | | | | | | | | |
| IFNG)- | −0.0247247 | −0.023472 | 0.06169218 | −0.0411081 | −0.0822324 | 0.01284627 | −0.1062134 | 0.26126482 | 0.27325428 |
| IFNGR1 | | | | | | | | | |
| GNB3)- | −0.150257 | −0.097143 | −0.2349197 | 0.05330961 | 0.18321521 | 0.09547971 | −0.0476724 | −0.077407 | −0.0786613 |
| GABBR2 | | | | | | | | | |
| ULBP1)- | −0.102135 | 0.05218767 | −0.0084377 | −0.2515234 | 0.28029132 | −0.1482262 | −0.2489785 | 0.23003953 | 0.25046113 |
| KLRK1 | | | | | | | | | |
| LAMA4)- | 0.41417702 | 0.05316381 | 0.45943542 | 0.03716027 | −0.3841871 | −0.3618514 | 0.42397892 | 0.36956522 | 0.06100132 |
| ITGA6 | | | | | | | | | |
| LAMA4)- | −0.0858395 | 0.21937481 | 0.46808793 | −0.1156317 | −0.2278464 | 0.31210674 | 0.32336081 | 0.00434783 | 0.07444005 |
| ITGAV | | | | | | | | | |
| LAMA4)- | −0.2460555 | 0.31918047 | 0.40758918 | −0.1925218 | −0.2809422 | 0.58422007 | 0.6115942 | −0.1615283 | 0.50737813 |
| ITGB1 | | | | | | | | | |
| HBEGF)- | 0.27701835 | −0.0766165 | 0.3753088 | 0.07760211 | 0.08339921 | 0.17575758 | 0.20935441 | 0.11212121 | −0.4329381 |
| CD44 | | | | | | | | | |
| HBEGF)- | −0.0578431 | −0.2576501 | 0.17711218 | −0.2299078 | 0.23434052 | 0.11080734 | 0.34037368 | 0.04499489 | −0.2017128 |
| EGFR | | | | | | | | | |
| HBEGF)- | 0.15718568 | −0.101782 | 0.07595771 | −0.3513834 | 0.27628458 | 0.26152833 | −0.0885375 | 0.04795784 | −0.1028986 |
| CD9 | | | | | | | | | |
| HBEGF)- | 0.00797128 | −0.0434138 | −0.1525742 | −0.0648221 | −0.0638999 | 0.02226614 | −0.1822134 | −0.1669302 | 0.35191041 |
| ERBB2 | | | | | | | | | |
| IL4)- | −0.2631648 | 0.23197577 | −0.068086 | 0.26166008 | 0.11015944 | 0.07484024 | −0.0334695 | 0.03814481 | 0.30711462 |
| IL13RA2 | | | | | | | | | |
| IL4)- | 0.06649752 | 0.02517464 | 0.08801424 | −0.0527009 | 0.12468697 | 0.0103429 | −0.0675498 | −0.1733917 | −0.2046113 |
| IL2RG | | | | | | | | | |
| IL4)- CD53 | 0.00764466 | 0.21273232 | −0.0804852 | 0.13623188 | −0.0386123 | 0.0942029 | 0.06799987 | 0.17312253 | 0.24953887 |
| IL4)- | −0.1751681 | −0.0611572 | 0.02135724 | −0.1152833 | −0.0417751 | −0.0516469 | 0.02556584 | −0.1629776 | −0.4046113 |
| IL13RA1 | | | | | | | | | |
| IL4)- IL4R | 0.27243972 | 0.1279204 | 0.06861038 | 0.32438486 | −0.2656199 | 0.03201581 | 0.21875354 | 0.26666667 | 0.03544254 |
| SEMA3G)- | −0.266124 | −0.0360485 | −0.159768 | 0.02536399 | −0.2718587 | −0.0735018 | −0.1763078 | −0.1002141 | 0.40052701 |
| NRP2 | | | | | | | | | |
| LTF)- LRP1 | −0.3028746 | 0.20654652 | 0.0888684 | 0.00171284 | 0.02345268 | 0.14493708 | −0.0716097 | 0.08939983 | −0.0796469 |
| LTF)- | −0.0191845 | 0.08445135 | −0.0430856 | −0.0023716 | 0.1478407 | 0.22280049 | −0.1705043 | 0.18103363 | −0.154287 |
| TFRC | | | | | | | | | |
| HRG)- | 0.05003792 | 0.10772327 | 0.06876094 | −0.0998715 | 0.15916203 | 0.19598801 | −0.284058 | −0.0351779 | 0.20065876 |
| ERBB2 | | | | | | | | | |
| HRG)- | 0.07898209 | 0.09328545 | −0.2980015 | 0.06034454 | 0.08687056 | −0.0096844 | −0.1732081 | −0.0305017 | 0.14229249 |
| FCGR1A | | | | | | | | | |
| HRG)- | 0.17701162 | −0.2103702 | 0.2712949 | −0.0069831 | −0.2913235 | 0.03267565 | 0.02477025 | 0.38906456 | −0.0905138 |
| ERBB3 | | | | | | | | | |
| TFPI)- | −0.1007346 | 0.31352527 | 0.2941564 | 0.26113307 | 0.22740448 | 0.08063507 | 0.08774704 | −0.2494153 | 0.15678524 |
| LRP1 | | | | | | | | | |
| TFPI)- | 0.23763094 | −0.2747875 | 0.22307135 | −0.2827404 | −0.0028986 | 0.00711462 | 0.01422925 | −0.0025033 | 0.15981555 |
| SDC4 | | | | | | | | | |
| TFPI)- F3 | 0.1322265 | −0.140396 | 0.20670563 | 0.04203037 | 0.23365757 | 0.17339175 | −0.1638372 | −0.1193715 | 0.18696268 |
| APOB)- | −0.0208237 | −0.0096211 | 0.13268064 | 0.05467721 | −0.0177289 | 0.10382082 | −0.3074541 | 0.26007905 | 0.27799736 |
| ITGAM | | | | | | | | | |
| APOB)- | −0.1187479 | 0.28244174 | 0.1679739 | −0.2939142 | −0.078474 | 0.18656126 | −0.0805166 | 0.00013175 | 0.10623085 |
| LRP6 | | | | | | | | | |
| APOB)- | 0.199743 | 0.04955519 | −0.1512295 | 0.12134387 | −0.0611572 | 0.06179387 | 0.11163834 | −0.0780658 | 0.12160738 |
| OLR1 | | | | | | | | | |
| APOB)- | −0.1286985 | 0.14906096 | 0.03775947 | −0.3335968 | −0.0492754 | 0.13873518 | 0.15612648 | 0.17496706 | −0.2918314 |
| LDLR | | | | | | | | | |
| APOB)- | −0.134412 | −0.1872158 | 0.00372532 | 0.28194993 | −0.2564678 | 0.35859934 | −0.1174571 | 0.22703166 | 0.32885375 |
| CALCR | | | | | | | | | |
| APOB)- | −0.0797417 | 0.02866557 | 0.27109369 | −0.030303 | −0.1231419 | −0.0474998 | −0.0983553 | 0.12122011 | 0.19828722 |
| MTTP | | | | | | | | | |
| APOB)- | −0.0294563 | 0.12738057 | 0.05713345 | −0.2479578 | 0.07628458 | 0.2 | 0.2259552 | 0.33610013 | −0.342029 |
| LSR | | | | | | | | | |
| APOB)- | −0.1619547 | −0.1094563 | 0.04407255 | 0.15322793 | −0.0900302 | 0.4329666 | 0.00679847 | 0.16384479 | 0.49090909 |
| ADRB2 | | | | | | | | | |
| APOB)- | 0.12751656 | −0.0223401 | 0.10456568 | 0.32537304 | −0.1035426 | 0.07832933 | −0.0663042 | 0.03405909 | 0.25323627 |
| TLR6 | | | | | | | | | |
| APOB)- | −0.0826057 | −0.0049423 | −0.001318 | −0.0111989 | −0.0220026 | 0.0431503 | −0.0362319 | −0.0334003 | −0.0316206 |
| LRP1 | | | | | | | | | |
| APOB)- | 0.21120265 | 0.21397036 | −0.1824803 | 0.09341238 | −0.0487596 | 0.06508564 | −0.144961 | 0.25889328 | 0.20382082 |
| ITGB2 | | | | | | | | | |
| APOB)- | 0.09397035 | 0.13752884 | −0.0740179 | −0.0092227 | −0.1559597 | 0.08036891 | −0.2934045 | 0.2884058 | 0.26521739 |
| TLR4 | | | | | | | | | |
| APOB)- | 0.36208773 | −0.2078419 | 0.16554065 | 0.26916996 | −0.1591276 | −0.2365032 | −0.0732712 | 0.0802398 | −0.2764163 |
| LRP8 | | | | | | | | | |
| PROC)- | −0.2341696 | −0.117744 | 0.30448836 | 0.10436502 | −0.2346989 | 0.11642234 | 0.18770186 | −0.085639 | 0.09301713 |
| ITGAM | | | | | | | | | |
| PROC)- | −0.1834065 | −0.173122 | 0.06472141 | −0.028002 | 0.10616845 | 0.01001482 | 0.10186802 | 0.05349144 | 0.03926219 |
| ITGB2 | | | | | | | | | |
| PROC)- | 0.01733857 | 0.08043251 | 0.1091991 | −0.1819859 | −0.0019146 | −0.076363 | −0.2361877 | 0.45006588 | 0.32846932 |
| THBD | | | | | | | | | |
| APOA1)- | −0.1430701 | 0.09569621 | 0.16744942 | 0.1993478 | −0.1845455 | 0.39798406 | −0.1927275 | 0.0292519 | 0.05415377 |
| ABCA1 | | | | | | | | | |
| APOA1)- | −0.0103059 | 0.18933815 | −0.0201494 | −0.0575758 | 0.07325428 | −0.0472991 | 0.10935802 | −0.0670641 | 0.05138509 |
| LDLR | | | | | | | | | |
| APOA1)- | −0.2491008 | −0.0175068 | −0.0673848 | −0.0067194 | 0.01554677 | 0.1987549 | 0.1100168 | −0.2065353 | 0.0501334 |
| LRP1 | | | | | | | | | |
| FGF23)- | −0.3033049 | 0.0137063 | 0.1977501 | −0.2506094 | 0.11250751 | 0.11282778 | −0.1792878 | 0.04927861 | −0.05639 |
| FGFR2 | | | | | | | | | |
| FGF23)- | −0.0043497 | −0.0038878 | −0.0313118 | −0.0971737 | 0.01673971 | −0.0324175 | −0.2435665 | 0.21149032 | −0.0023715 |
| PHEX | | | | | | | | | |
| FGF23)- KL | 0.11395242 | 0.19940036 | −0.1056466 | −0.1928322 | 0.10338124 | 0.01172866 | 0.24807226 | 0.02978093 | 0.21198946 |
| FGF23)- | −0.1460956 | −0.0303779 | 0.04540359 | 0.15132749 | −0.1546069 | 0.03073426 | 0.01265781 | 0.31470075 | 0.04110672 |
| FGFR4 | | | | | | | | | |
| FGF23)- | 0.06211078 | −0.0032289 | −0.0452104 | 0.05777719 | 0.06496673 | −0.0821913 | 0.03874028 | −0.245339 | 0.03952569 |
| FGFR1 | | | | | | | | | |
| FGF23)- | −0.1289579 | −0.0319605 | −0.1365674 | −0.1546925 | 0.01281671 | 0.13083866 | −0.0377557 | 0.0685112 | −0.1211502 |
| FGFR3 | | | | | | | | | |
| TGFB3)- | −0.0216783 | −0.0414402 | −0.0157569 | 0.01291215 | −0.0634848 | −0.0674024 | 0.12413069 | −0.2349453 | 0.07140975 |
| ENG | | | | | | | | | |
| TGFB3)- | −0.1934974 | 0.25160589 | 0.31834503 | 0.13182252 | 0.12556408 | −0.1249712 | 0.38827404 | −0.1210804 | 0.33254282 |
| ITGB1 | | | | | | | | | |
| TGFB3)- | 0.21942944 | −0.0119248 | 0.10819359 | −0.1432195 | −0.1683413 | −0.3315436 | 0.05270787 | 0.1467769 | −0.0700922 |
| TGFBR2 | | | | | | | | | |
| TGFB3)- | −0.224631 | −0.0763301 | 0.09672212 | −0.0469085 | 0.03880489 | −0.1007445 | 0.10626524 | −0.0776149 | 0.24626128 |
| ITGB3 | | | | | | | | | |
| TGFB3)- | 0.10430944 | −0.0080377 | −0.0799671 | −0.1146942 | 0.08717712 | −0.3140278 | −0.0919843 | 0.12722781 | −0.0321476 |
| ACVRL1 | | | | | | | | | |
| TGFB3)- | 0.15442896 | −0.0239813 | 0.32849096 | −0.0442702 | −0.0179848 | 0.07813169 | 0.18906456 | 0.05849802 | 0.16258235 |
| ITGB6 | | | | | | | | | |
| TGFB3)- | −0.3809132 | 0.3359357 | 0.18104556 | −0.0685135 | 0.06370434 | 0.19760854 | 0.171278 | −0.344346 | 0.3541502 |
| ITGB5 | | | | | | | | | |
| IAPP)- | −0.0120637 | 0.09663812 | 0.11433661 | 0.15132251 | 0.0119342 | 0.20195665 | 0.20030336 | −0.0283277 | 0.09314888 |
| PTH1R | | | | | | | | | |
| LAPP)- | 0.06684247 | 0.0938695 | 0.04543057 | −0.103429 | −0.0173965 | 0.05052867 | −0.0133764 | −0.1094862 | 0.22938076 |
| RAMP2 | | | | | | | | | |
| LAPP)- | 0.2383731 | −0.1220292 | 0.40329761 | 0.01350639 | −0.1410493 | 0.00757626 | −0.031043 | 0.06317731 | −0.3289521 |
| VIPR1 | | | | | | | | | |
| LAPP)- | −0.0381053 | 0.09914307 | 0.18823995 | −0.1780691 | 0.14844113 | −0.0719462 | −0.0549056 | 0.19349738 | 0.21449275 |
| CALCR | | | | | | | | | |
| LAPP)- | −0.0672424 | −0.1015162 | −0.0626136 | −0.0735861 | 0.29706325 | 0.02016009 | 0.20560417 | 0.09486791 | 0.25335968 |
| ADRB2 | | | | | | | | | |
| IAPP)- | −0.0837866 | −0.1930126 | −0.2754641 | 0.13722455 | 0.06524748 | 0.12701759 | −0.0095561 | 0.14638163 | 0.42239789 |
| RAMP1 | | | | | | | | | |
| IAPP)- | −0.1569196 | −0.0805564 | 0.04438582 | 0.05929244 | −0.1584498 | 0.28619906 | 0.01014994 | 0.38110299 | 0.09506242 |
| ADRB3 | | | | | | | | | |
| LAPP)- | 0.0104153 | 0.10771261 | 0.09507798 | −0.1202279 | 0.04853058 | 0.23314338 | 0.33439859 | 0.02318841 | 0.01330698 |
| GPR84 | | | | | | | | | |
| TNFSF10)- | 0.33920748 | 0.11080734 | 0.13467747 | 0.04967062 | 0.03241641 | 0.03320158 | −0.1447256 | 0.08623472 | 0.15514345 |
| TNFRSF10D | | | | | | | | | |
| TNFSF10)- | −0.0525137 | 0.39632399 | 0.32747885 | 0.31778656 | 0.4062212 | 0.06654587 | −0.1543151 | 0.08743494 | −0.3930301 |
| RIPK1 | | | | | | | | | |
| TNFSF10)- | 0.01870944 | 0.10270431 | 0.07589683 | 0.21620553 | −0.1362409 | −0.0711462 | 0.03294133 | 0.19111301 | −0.3537666 |
| TNFRSF10B | | | | | | | | | |
| TNFSF10)- | −0.0233899 | −0.2537967 | 0.39855049 | −0.1769667 | 0.14929503 | 0.04980893 | 0.10541921 | 0.04486905 | −0.159183 |
| TNFRSF10C | | | | | | | | | |
| TNFSF10)- | −0.0142302 | −0.1559999 | 0.07352263 | −0.2545455 | 0.15244244 | 0.09071445 | −0.341788 | −0.3746624 | 0.55502487 |
| TNFRSF11B | | | | | | | | | |
| INHBA)- | 0.10346305 | −0.0505996 | −0.0245391 | −0.0127821 | 0.13622282 | 0.41874261 | −0.1926846 | 0.02721671 | 0.01027803 |
| ACVR1 | | | | | | | | | |
| INHBA)- | −0.0650942 | 0.14598155 | 0.15920107 | 0.20679206 | −0.1233437 | −0.2387745 | 0.1156927 | −0.0396627 | 0.15401845 |
| ENG | | | | | | | | | |
| INHBA)- | 0.10395257 | −0.0607378 | 0.21601502 | 0.28406733 | −0.1940839 | −0.3870351 | 0.14308772 | 0.17891963 | −0.1259552 |
| SMAD3 | | | | | | | | | |
| INHBA)- | 0.15593399 | −0.1450593 | −0.1459123 | 0.08432425 | 0.20991236 | 0.08518348 | −0.2056217 | 0.11383774 | −0.0881423 |
| BAMBI | | | | | | | | | |
| INHBA)- | 0.00698287 | −0.1220026 | 0.22805747 | −0.1660134 | 0.00474449 | −0.1894661 | 0.20565367 | 0.00223979 | −0.2076416 |
| ACVR1B | | | | | | | | | |
| INHA)- | −0.1875 | 0.0398774 | −0.2399526 | −0.0324164 | 0.07180559 | −0.1517728 | −0.1598722 | 0.15394755 | 0.20932301 |
| ACVR1 | | | | | | | | | |
| PI3)- PLD2 | −0.11681 | −0.3040485 | 0.2201228 | 0.08537831 | −0.098991 | −0.0565236 | −0.002803 | −0.0727321 | 0.09921602 |
| LYPD3)- | −0.0935399 | 0.0134476 | −0.0406724 | −0.1322837 | 0.2507329 | 0.18880727 | 0.0201581 | 0.12819499 | −0.2092227 |
| AGR2 | | | | | | | | | |
| EFNB2)- | −0.1764454 | −0.3351672 | 0.31730234 | −0.2432228 | 0.1638707 | 0.07806581 | −0.1793672 | 0.2164833 | 0.37597997 |
| EPHB6 | | | | | | | | | |
| EFNB2)- | −0.1029121 | −0.0951377 | 0.16366638 | −0.1951252 | 0.24869792 | −0.1693017 | 0.02109914 | −0.0006588 | −0.1344577 |
| PECAM1 | | | | | | | | | |
| EFNB2)- | 0.02161023 | −0.046055 | 0.29519542 | 0.17602688 | −0.0396046 | −0.2778656 | −0.1113052 | 0.09526005 | −0.0459187 |
| EPHB4 | | | | | | | | | |
| EFNB2)- | −0.2196086 | −0.1420477 | 0.1267833 | 0.12714097 | 0.02602537 | 0.13986429 | −0.1633393 | 0.33596864 | 0.09585296 |
| RHBDL2 | | | | | | | | | |
| EFNB2)- | 0.00448076 | 0.21465279 | 0.15963597 | 0.17931489 | 0.16818929 | 0.04651469 | −0.2299982 | −0.3051227 | −0.2099542 |
| EPHA3 | | | | | | | | | |
| EFNB2)- | −0.2696452 | 0.20648307 | 0.12383024 | 0.43926219 | −0.0399315 | −0.0922418 | −0.0052717 | −0.0330764 | −0.0221351 |
| EPHB1 | | | | | | | | | |
| EFNB2)- | −0.1529896 | 0.14995388 | 0.03593328 | 0.28748353 | −0.0768673 | −0.0247044 | 0.18755979 | −0.1168061 | −0.143483 |
| EPHB3 | | | | | | | | | |
| EFNB2)- | −0.0807801 | 0.06931085 | −0.0600349 | −0.0828722 | 0.05528284 | −0.0585019 | 0.20964023 | −0.4022137 | −0.0246385 |
| EPHB2 | | | | | | | | | |
| EFNB2)- | 0.14176498 | −0.28146 | −0.0140367 | −0.1857708 | 0.00935657 | 0.14510709 | −0.2346798 | 0.13035026 | 0.32339669 |
| EPHA4 | | | | | | | | | |
| BMP4)- | −0.0754966 | 0.17510216 | 0.21750733 | 0.29345285 | 0.0340647 | 0.09525692 | −0.3533636 | 0.25862978 | −0.2087713 |
| LRP6 | | | | | | | | | |
| BMP4)- | −0.1617366 | 0.07556244 | 0.32380262 | −0.2927536 | −0.2031029 | 0.02773477 | −0.1262887 | −0.0020422 | 0.03570487 |
| BMPR2 | | | | | | | | | |
| BMP4)- | −0.1449371 | −0.2316941 | −0.0968688 | −0.0847167 | −0.0903069 | 0.10738167 | 0.16571639 | −0.0565236 | −0.1496706 |
| BMPR1A | | | | | | | | | |
| BMP4)- | −0.168539 | −0.0040845 | 0.17433585 | 0.10065876 | 0.01641452 | 0.04585584 | −0.1310525 | −0.018316 | −0.2715415 |
| BMPR1B | | | | | | | | | |
| TNFSF9)- | −0.0253807 | −0.0711509 | 0.15138235 | −0.0426934 | −0.0278794 | −0.0657964 | −0.2935188 | 0.03150957 | −0.0246377 |
| TNFRSF9 | | | | | | | | | |
| TNFSF9)- | −0.0974405 | 0.03214968 | −0.1132013 | 0.16787456 | −0.009489 | −0.0539615 | −0.0770219 | 0.00751013 | −0.2166008 |
| PVR | | | | | | | | | |
| TNFSF9)- | 0.01021181 | 0.15831082 | 0.01997825 | 0.26090394 | 0.07470906 | −0.0791303 | −0.1690457 | −0.0677888 | −0.2677207 |
| TRAF2 | | | | | | | | | |
| C3)- | 0.09631728 | −0.109691 | 0.00369101 | 0.29986825 | −0.134713 | 0.20500659 | 0.10894353 | −0.0067194 | 0.07944664 |
| ITGAM | | | | | | | | | |
| C3)- CD46 | −0.009948 | 0.1400336 | 0.05738191 | −0.2071873 | 0.02226614 | 0.14769433 | 0.3057971 | 0.38102767 | −0.1768174 |
| C3)- C5AR2 | −0.0076097 | 0.01943475 | 0.0751845 | 0.08629776 | −0.1934506 | 0.27281112 | 0.0152204 | 0.1090981 | −0.1162055 |
| C3)- CR2 | −0.0552756 | 0.04315172 | −0.3365441 | 0.22384717 | 0.26907244 | 0.04888172 | 0.11346031 | −0.1004644 | 0.11475626 |
| C3)- | −0.0735226 | −0.1683247 | 0.18804574 | 0.32358366 | −0.2320846 | 0.37720685 | −0.0765049 | 0.29512516 | 0.11172596 |
| ITGAX | | | | | | | | | |
| C3)- C3AR1 | −0.2268999 | −0.235391 | 0.32591647 | 0.1627141 | −0.1089064 | 0.35238315 | 0.06473731 | 0.31035278 | 0.18076416 |
| C3)- | −0.4397668 | 0.32584492 | −0.081236 | −0.114888 | 0.08966335 | 0.30218387 | −0.0015811 | 0.00928884 | −0.083531 |
| ADRA2A | | | | | | | | | |
| C3)- | 0.00415047 | −0.3921207 | 0.0981684 | 0.29130435 | 0.00151525 | 0.21093544 | 0.21121286 | 0.21791831 | 0.09077734 |
| IFITM1 | | | | | | | | | |
| C3)- CR1 | −0.1666886 | −0.1506687 | 0.10987346 | 0.03820817 | 0.07935149 | −0.0088939 | 0.05450472 | −0.1556756 | −0.0167325 |
| C3)- ITGB2 | −0.3460702 | −0.3040385 | 0.40772957 | 0.34347826 | −0.3106777 | 0.34993412 | −0.0097519 | 0.17918314 | 0.15059289 |
| C3)- CD81 | 0.0665393 | −0.1772844 | 0.27708581 | −0.0635046 | −0.1405601 | −0.1151515 | 0.19341022 | −0.0649539 | 0.13992095 |
| VASP)- | 0.11173332 | 0.38094611 | −0.1876403 | 0.41085782 | −0.1453635 | 0.11963898 | 0.1233388 | −0.1666502 | −0.420425 |
| CXCR2 | | | | | | | | | |
| SLURP1)- | −0.2093575 | −0.0101479 | 0.15756698 | 0.07259552 | 0.22906647 | −0.1144306 | 0.37413532 | −0.1458546 | −0.0521739 |
| CHRNA7 | | | | | | | | | |
| TPH1)- | −0.0213832 | 0.05297839 | 0.30891292 | −0.0766899 | −0.1019789 | −0.130588 | −0.143253 | 0.18162654 | 0.39051383 |
| HTR1A | | | | | | | | | |
| TPH1)- | −0.0046125 | −0.1515551 | 0.19884641 | 0.09434708 | −0.0663064 | 0.30003953 | −0.0512719 | 0.11106719 | 0.07852437 |
| HTR1F | | | | | | | | | |
| TPH1)- | −0.1896415 | 0.22410386 | 0.19961119 | −0.0910528 | −0.1638059 | 0.22163658 | 0.2724907 | −0.1030303 | 0.10447958 |
| HTR1D | | | | | | | | | |
| TPH1)- | −0.0608418 | 0.29454403 | 0.04517894 | −0.1329556 | −0.1568369 | 0.08126545 | −0.3368255 | 0.10128835 | 0.08339921 |
| HTR2C | | | | | | | | | |
| TPH1)- | 0.06358934 | 0.11673421 | 0.17119789 | −0.0052051 | −0.1329204 | 0.14106408 | 0.11998814 | −0.0393294 | 0.27102342 |
| HTR1B | | | | | | | | | |
| TPH1)- | −0.1467449 | −0.0540327 | 0.13534529 | 0.15772829 | 0.00342601 | 0.18263276 | 0.2886693 | 0.31370224 | −0.0395257 |
| HTR2A | | | | | | | | | |
| TPH1)- | 0.05060622 | −0.0312335 | 0.09572785 | 0.24074318 | −0.1308504 | 0.20121228 | −0.2057079 | 0.02714097 | −0.0616601 |
| HTR2B | | | | | | | | | |
| TPH1)- | −0.0264909 | −0.1983395 | 0.26524089 | 0.13756753 | 0.08289951 | 0.1618897 | −0.1017329 | 0.24685421 | 0.08616601 |
| HTR1E | | | | | | | | | |
| LMAN1)- | −0.0931936 | 0.07815339 | 0.2680039 | −0.0135705 | −0.2390273 | 0.15369413 | 0.30057996 | 0.02957937 | 0.55744401 |
| MCFD2 | | | | | | | | | |
| APOE)- | 0.14553001 | −0.1756022 | −0.0140683 | 0.10273815 | −0.0090268 | −0.0411067 | 0.12551888 | −0.0346509 | 0.08474744 |
| LRP6 | | | | | | | | | |
| APOE)- | 0.18275249 | −0.1387443 | 0.20172607 | 0.13017128 | −0.2085639 | −0.2131752 | −0.085112 | −0.1172596 | 0.09591568 |
| LDLR | | | | | | | | | |
| APOE)- | −0.0357119 | 0.13499358 | −0.1327492 | −0.1772786 | 0.05744401 | 0.08670444 | 0.08484848 | 0.09197523 | −0.2083731 |
| LRP5 | | | | | | | | | |
| APOE)- | 0.06357886 | −0.0916098 | −0.0653836 | 0.37096084 | −0.1319449 | 0.17385862 | −0.08324 | 0.13782199 | 0.34665173 |
| CHRNA4 | | | | | | | | | |
| APOE)- | −0.065125 | −0.0059292 | 0.18386347 | 0.02239789 | −0.2177586 | 0.13808096 | −0.2011348 | 0.09598472 | 0.00289855 |
| TREM2 | | | | | | | | | |
| APOE)- | −0.1336056 | −0.0696357 | −0.0867646 | −0.0100132 | 0.13886693 | 0.19354414 | 0.26205534 | 0.25704875 | −0.071805 |
| LSR | | | | | | | | | |
| APOE)- | 0.02286053 | 0.19019698 | 0.13900784 | −0.1731225 | −0.1251647 | 0.05783926 | −0.0544137 | 0.09670619 | −0.2578393 |
| SORL1 | | | | | | | | | |
| APOE)- | −0.1860588 | 0.01390124 | 0.12152186 | 0.05882934 | −0.0686359 | 0.12174715 | 0.00151645 | 0.07088741 | 0.10441714 |
| ABCA1 | | | | | | | | | |
| APOE)- | 0.06970156 | 0.02582515 | 0.01803377 | −0.0546772 | −0.0189227 | −0.1043478 | −0.0735149 | −0.0487484 | −0.0758893 |
| SCARB1 | | | | | | | | | |
| APOE)- | 0.26754068 | −0.1264905 | −0.0471046 | 0.15019763 | 0.0198946 | −0.4963109 | 0.08498024 | −0.5501976 | 0.2201581 |
| SDC2 | | | | | | | | | |
| APOE)- | −0.0573179 | 0.10316885 | −0.022141 | −0.1171278 | −0.0434224 | 0.15698804 | −0.0353837 | 0.09163675 | −0.1234519 |
| LRP8 | | | | | | | | | |
| APOC2)- | −0.1336056 | 0.19645563 | 0.02944858 | −0.2068625 | 0.11734074 | 0.17924128 | −0.1108037 | 0.03201581 | 0.16086957 |
| LDLR | | | | | | | | | |
| GDF15)- | 0.14704042 | −0.037353 | −0.0449527 | −0.1809019 | 0.23537558 | 0.01350506 | −0.2644432 | 0.0133733 | 0.23847953 |
| GFRAL | | | | | | | | | |
| GDF15)- | −0.1641029 | −0.0354414 | 0.17112162 | 0.00487484 | −0.0106786 | −0.1424948 | −0.0074487 | 0.16798972 | −0.001581 |
| RET | | | | | | | | | |
| LAMA5)- | −0.3948747 | 0.43835436 | −0.0731908 | 0.35338296 | −0.2958034 | −0.5853482 | 0.45562949 | 0.76777126 | −0.3738059 |
| SDC1 | | | | | | | | | |
| LAMA5)- | 0.41898613 | −0.1060641 | 0.44072598 | −0.0020423 | 0.19177811 | 0.2641149 | −0.0610053 | −0.1143685 | −0.1515251 |
| ITGA6 | | | | | | | | | |
| LAMA5)- | 0.36799631 | −0.1925623 | 0.15626853 | −0.4831675 | 0.44602563 | 0.73226168 | −0.3483216 | −0.5633441 | 0.63937018 |
| BCAM | | | | | | | | | |
| LAMA5)- | −0.3747818 | 0.02865707 | 0.13287658 | 0.05751367 | 0.15007576 | −0.6462218 | −0.0814942 | 0.69108637 | −0.086567 |
| ITGB1 | | | | | | | | | |
| LAMA5)- | 0.46009421 | −0.2901005 | 0.39177839 | −0.5108388 | 0.54068121 | 0.50029646 | −0.3077937 | −0.3587852 | 0.47789418 |
| ITGA2 | | | | | | | | | |
| F12)- | −0.1926203 | −0.0700991 | 0.32948881 | −0.2354531 | 0.19740022 | −0.1463358 | −0.0281681 | −0.1536054 | 0.04743239 |
| GP1BA | | | | | | | | | |
| JAG1)- | −0.0627182 | 0.02226834 | −0.1081758 | 0.18823917 | −0.1154302 | −0.1001449 | −0.1300395 | −0.1076416 | 0.21733259 |
| CD46 | | | | | | | | | |
| JAG1)- | 0.01924344 | −0.2281441 | −0.1236203 | −0.1108183 | −0.1097235 | 0.06188625 | 0.21618951 | −0.1330478 | −0.4797101 |
| NOTCH4 | | | | | | | | | |
| JAG1)- | −0.292519 | −0.1665459 | 0.11391571 | −0.1184609 | 0.00876586 | −0.0631856 | −0.1310752 | 0.19493396 | 0.04532279 |
| NOTCH1 | | | | | | | | | |
| JAG1)- | −0.071678 | 0.03017327 | 0.17711594 | 0.04941363 | 0.16546407 | 0.12939781 | 0.0679295 | −0.072859 | 0.20461133 |
| NOTCH2 | | | | | | | | | |
| JAG1)- | −0.1189143 | 0.31550168 | 0.27515894 | −0.2018711 | −0.0895404 | 0.47107656 | 0.01047465 | 0.19262187 | 0.0259552 |
| NOTCH3 | | | | | | | | | |
| CRP)- | 0.07836289 | −0.0969453 | 0.07371024 | −0.0404493 | −0.1740535 | 0.06140062 | 0.11730979 | 0.03129323 | 0.37899799 |
| OLR1 | | | | | | | | | |
| CRP)- CR1 | −0.1655627 | −0.1208027 | −0.077836 | 0.18808261 | 0.04771792 | 0.04163784 | 0.00326248 | −0.1411865 | 0.31259264 |
| TSHB)- | −0.044789 | −0.0325187 | −0.1337845 | 0.02081824 | 0.12185155 | 0.14803834 | 0.02980155 | 0.04420435 | −0.0151515 |
| PTH1R | | | | | | | | | |
| TSHB)- | 0.08106867 | −0.083185 | 0.32515024 | 0.1513095 | −0.2640477 | 0.0608097 | −0.3800956 | 0.18913666 | 0.09039101 |
| VIPR1 | | | | | | | | | |
| TSHB)- | −0.1143508 | 0.1362753 | −0.0209834 | 0.11298505 | 0.12515287 | 0.13315325 | 0.15359258 | 0.24296726 | 0.02002635 |
| ADRB2 | | | | | | | | | |
| TSHB)- | −0.2569686 | 0.07104227 | 0.0654656 | 0.12758178 | 0.24977757 | 0.01858197 | −0.352348 | 0.15879291 | 0.10659113 |
| ADRB3 | | | | | | | | | |
| TSHB)- | −0.2274993 | 0.20441295 | −0.0103583 | −0.1378879 | 0.07367138 | 0.21806443 | 0.15668962 | −0.0511199 | 0.12252964 |
| GPR84 | | | | | | | | | |
| FST)- | −0.1584896 | 0.24599672 | 0.15957693 | 0.09124448 | −0.0558026 | −0.0855486 | 0.27748864 | −0.2729429 | 0.35949801 |
| BMPR2 | | | | | | | | | |
| FST)- | −0.0968826 | −0.107743 | 0.24250719 | 0.10527703 | −0.0551472 | 0.334124 | −0.0535962 | −0.0126503 | 0.00540202 |
| BMPR1B | | | | | | | | | |
| GRP)- | −0.0852662 | −0.011531 | 0.1605813 | 0.23947559 | −0.0736714 | 0.04223079 | 0.27121772 | −0.02095 | 0.28136632 |
| GRPR | | | | | | | | | |
| SEMA6A)- | −0.2554927 | −0.2695253 | 0.23766178 | 0.22233934 | −0.1727126 | 0.04401542 | −0.0172641 | 0.24240628 | −0.0210804 |
| PLXNA4 | | | | | | | | | |
| SEMA6A)- | −0.1953168 | 0.16517709 | 0.14454424 | 0.07207326 | −0.167946 | −0.0285912 | −0.2305969 | −0.0424242 | −0.3064001 |
| PLXNA2 | | | | | | | | | |
| LACRT)- | −0.0198372 | 0.10551291 | 0.07242891 | −0.1274045 | −0.226087 | −0.1689065 | −0.1639711 | 0.00191047 | 0.08491716 |
| SDC1 | | | | | | | | | |
| GDF11)- | −0.2560048 | −0.0229317 | −0.0003955 | −0.2815732 | 0.0340033 | 0.01983003 | 0.12249605 | −0.0166008 | 0.09552042 |
| ACVR1B | | | | | | | | | |
| GDF11)- | −0.0712356 | −0.0652367 | 0.08342669 | 0.15244746 | 0.0193695 | 0.0049412 | 0.0653513 | 0.04400672 | 0.12187088 |
| BMPR2 | | | | | | | | | |
| GDF11)- | −0.0329489 | −0.1499127 | 0.22431756 | 0.13887608 | 0.07455751 | 0.0577791 | −0.0281467 | −0.0606081 | 0.04334651 |
| BMPR1A | | | | | | | | | |
| GDF11)- | 0.15382081 | −0.0821719 | 0.08740522 | −0.0505962 | 0.18301088 | −0.2139421 | 0.02214971 | 0.03781789 | 0.22147563 |
| BMPR1B | | | | | | | | | |
| LAMC1)- | 0.24482804 | −0.2833048 | 0.2224272 | 0.19552686 | −0.3108798 | −0.2635792 | 0.33741765 | 0.3140975 | −0.284058 |
| ITGA6 | | | | | | | | | |
| LAMC1)- | −0.1529288 | 0.14099354 | 0.10945274 | −0.190586 | −0.0185149 | 0.1871397 | 0.00480975 | −0.0949997 | 0.04822134 |
| ITGA1 | | | | | | | | | |
| LAMC1)- | −0.0353825 | −0.1102912 | 0.09884605 | 0.01844593 | 0.09006695 | 0.24785058 | 0.10331645 | −0.2274853 | −0.1814229 |
| ITGA7 | | | | | | | | | |
| LAMC1)- | −0.1826328 | −0.1557517 | 0.10090292 | 0.08926513 | −0.1432667 | 0.36707401 | 0.09660627 | −0.2653491 | −0.1765481 |
| ITGAV | | | | | | | | | |
| LAMC1)- | −0.4625115 | 0.36170774 | 0.44696271 | −0.1988867 | −0.2367008 | 0.60153497 | 0.34822134 | −0.5591568 | 0.3088274 |
| ITGB1 | | | | | | | | | |
| LAMC1)- | 0.23441824 | −0.2920401 | 0.22664383 | 0.38926042 | −0.3349254 | −0.4340064 | 0.27509881 | 0.33557312 | −0.286599 |
| ITGA2 | | | | | | | | | |
| IL1RN)- | −0.1405648 | 0.03926477 | −0.1520113 | −0.1349144 | −0.056004 | −0.0052717 | 0.38201285 | 0.21066852 | 0.10013175 |
| IL1R1 | | | | | | | | | |
| IL1RN)- | 0.39238422 | −0.4596482 | 0.20865182 | −0.1761528 | −0.2901759 | −0.1603426 | −0.0026356 | −0.2992095 | 0.55401845 |
| IL1R2 | | | | | | | | | |
| ORM1)- | −0.2468266 | 0.04932575 | −0.0223982 | 0.04665569 | −0.1082872 | 0.3667216 | 0.2070193 | −0.0850489 | 0.02944761 |
| CCR5 | | | | | | | | | |
| CCN3)- | −0.1585555 | −0.124477 | 0.10829159 | −0.1554728 | 0.17000527 | −0.0738874 | −0.0457945 | 0.10218064 | 0.48682477 |
| PLXNA1 | | | | | | | | | |
| CCN3)- | 0.11222406 | −0.0860598 | 0.27129202 | −0.0039527 | 0.08461843 | −0.2077871 | −0.0371017 | 0.0282618 | −0.0631094 |
| NOTCH1 | | | | | | | | | |
| CCL21)- | −0.2618295 | 0.14834098 | −0.1669358 | −0.0798445 | 0.20436802 | 0.34636669 | 0.17953026 | 0.16542592 | −0.1492803 |
| ADRA2A | | | | | | | | | |
| TGS1)- | −0.0402623 | −0.0687247 | −0.0529771 | −0.070224 | 0.10612765 | 0.2776204 | −0.0434367 | −0.0338648 | 0.17405626 |
| RXRA | | | | | | | | | |
| MMP7)- | 0.29710145 | −0.2963109 | 0.00750988 | −0.3624506 | 0.01936759 | 0.38629776 | −0.1470356 | −0.0807642 | 0.53175231 |
| CD44 | | | | | | | | | |
| MMP7)- | −0.0700922 | 0.07233202 | 0.07312253 | 0.08642951 | 0.07338603 | −0.0455863 | 0.18129117 | 0.23939394 | −0.213834 |
| SDC1 | | | | | | | | | |
| MMP7)- | −0.1811594 | 0.0198946 | −0.055863 | 0.0027668 | −0.0472991 | −0.1533597 | 0.02108037 | 0.06007905 | 0.08432148 |
| CD151 | | | | | | | | | |
| THBS1)- | 0.29525692 | 0.32002635 | −0.2623081 | −0.2714097 | 0.18401109 | −0.1990777 | −0.3086404 | 0.35322793 | 0.37338603 |
| ITGA4 | | | | | | | | | |
| THBS1)- | 0.1113307 | −0.0341238 | 0.13452354 | 0.07220026 | −0.0480912 | −0.242029 | 0.09341546 | 0.14664032 | −0.0094862 |
| CD47 | | | | | | | | | |
| THBS1)- | 0.10566535 | 0.01976285 | −0.1607378 | −0.1296443 | 0.0911726 | −0.0862978 | −0.1125165 | 0.18102767 | −0.0102767 |
| SDC1 | | | | | | | | | |
| THBS1)- | −0.085639 | 0.0201581 | 0.13886693 | 0.11844532 | 0.04044796 | 0.09894598 | 0.27918314 | −0.084585 | 0.17865613 |
| ITGB1 | | | | | | | | | |
| THBS1)- | 0.21438925 | −0.1766198 | 0.08932806 | 0.22978359 | −0.1544137 | −0.1417842 | 0.14453228 | 0.3292924 | −0.3810402 |
| LRP5 | | | | | | | | | |
| THBS1)- | −0.1182208 | 0.01870883 | 0.12037159 | 0.03952569 | −0.14073 | 0.13133444 | 0.13638161 | −0.0979901 | 0.00974967 |
| ITGA2B | | | | | | | | | |
| THBS1)- | −0.0818317 | 0.07648725 | −0.1130509 | −0.0520456 | 0.14111602 | 0.07919618 | −0.1351868 | −0.043156 | 0.14553001 |
| ITGB3 | | | | | | | | | |
| THBS1)- | 0.07549407 | 0.12766798 | 0.09222661 | −0.0179183 | −0.1708827 | −0.1936759 | 0.16047431 | 0.13175231 | 0.1541502 |
| SDC4 | | | | | | | | | |
| THBS1)- | 0.00395257 | 0.12187088 | −0.1568538 | −0.2766798 | 0.31186139 | −0.1192358 | −0.3061912 | 0.1201581 | 0.18695652 |
| SCARB1 | | | | | | | | | |
| THBS1)- | 0.35243742 | −0.4587615 | 0.26745718 | 0.40316206 | −0.2504611 | −0.3498024 | 0.32371542 | 0.38880105 | −0.5320158 |
| ITGA6 | | | | | | | | | |
| THBS1)- | 0.17003195 | 0.06969697 | 0.27813828 | 0.0657444 | −0.331368 | −0.1699002 | 0.17576337 | 0.12477354 | 0.12648221 |
| TNFRSF11B | | | | | | | | | |
| THBS1)- | −0.1382786 | 0.01172634 | 0.04391114 | −0.0048091 | −0.074438 | 0.0391976 | 0.11768976 | −0.2223393 | 0.02470437 |
| CD36 | | | | | | | | | |
| ADAM10)- | −0.0393307 | −0.0158772 | 0.13375293 | 0.08735466 | −0.0100501 | 0.09993742 | −0.1904511 | 0.06864295 | −0.0013175 |
| GPNMB | | | | | | | | | |
| ADAM10)- | −0.1237276 | −0.2184669 | −0.1633817 | −0.1471111 | −0.2086945 | −0.0950656 | 0.07764816 | 0.14835798 | 0.01278039 |
| TSPAN5 | | | | | | | | | |
| ADAM10)- | −0.107517 | −0.0814283 | −0.1487582 | −0.0343226 | −0.1216114 | −0.0686452 | 0.23530962 | 0.21791831 | 0.01884058 |
| TSPAN15 | | | | | | | | | |
| ADAM10)- | −0.3945583 | 0.06186178 | −0.1267541 | −0.0329392 | −0.0929543 | −0.239863 | 0.25586298 | 0.3859025 | 0.00144928 |
| CD44 | | | | | | | | | |
| ADAM10)- | −0.1000428 | 0.0824165 | 0.02389743 | 0.19414342 | −0.2344837 | 0.17675736 | −0.1564272 | 0.1750387 | −0.1508564 |
| CADM1 | | | | | | | | | |
| ADAM10)- | −0.0744474 | −0.0770143 | 0.07206168 | 0.01166046 | 0.00046129 | 0.07088741 | 0.08665283 | −0.1749069 | −0.0450593 |
| TSPAN14 | | | | | | | | | |
| ADAM10)- | −0.2730178 | −0.0786613 | −0.0312716 | −0.0934813 | −0.0126862 | −0.1401278 | −0.174042 | 0.29579367 | −0.0125165 |
| NOTCH1 | | | | | | | | | |
| ADAM10)- | 0.03863143 | 0.10356413 | 0.02675277 | 0.02266214 | −0.012124 | 0.2383731 | 0.03195625 | −0.1398814 | −0.2606061 |
| IL6R | | | | | | | | | |
| ADAM10)- | 0.05501021 | −0.411819 | 0.14572765 | −0.2068579 | 0.1938799 | 0.25890181 | −0.3470356 | −0.344664 | 0.54018445 |
| MET | | | | | | | | | |
| ADAM10)- | −0.0579471 | −0.2019304 | −0.0171431 | −0.1139733 | −0.0971517 | −0.0702726 | 0.06619419 | 0.03638762 | 0.28433084 |
| TSPAN17 | | | | | | | | | |
| ADAM10)- | 0.01139883 | 0.10784637 | 0.00098941 | 0.13630225 | 0.01517051 | 0.05758524 | −0.2608583 | −0.2130716 | −0.3180501 |
| EPHA3 | | | | | | | | | |
| ADAM10)- | −0.0190401 | 0.19270044 | −0.0895897 | 0.23722784 | −0.1276183 | −0.0081033 | −0.0251996 | 0.08254554 | −0.0140975 |
| TREM2 | | | | | | | | | |
| ADAM10)- | −0.2218855 | −0.0538902 | 0.04819317 | −0.1442076 | −0.1704328 | 0.03023815 | 0.0659442 | 0.15388669 | 0.15625823 |
| AXL | | | | | | | | | |
| COL8A1)- | −0.109164 | 0.3173913 | 0.17282161 | −0.043083 | 0.04928348 | −0.2101588 | 0.16708944 | −0.0542196 | 0.28695652 |
| ITGA1 | | | | | | | | | |
| COL8A1)- | −0.0495389 | −0.1081829 | −0.0006588 | 0.27078667 | −0.2019763 | −0.1466403 | 0.0886693 | 0.06627141 | −0.1216234 |
| ITGA2 | | | | | | | | | |
| LYZ)- | 0.243083 | 0.2057971 | −0.0723749 | 0.07391304 | 0.06327863 | 0.03254282 | 0.18693563 | −0.330303 | −0.3046113 |
| ITGAL | | | | | | | | | |
| VWF)- | −0.0780941 | 0.00335991 | 0.25067536 | 0.07801278 | −0.2337397 | −0.02587 | 0.15048096 | 0.01621087 | −0.0542819 |
| ITGA2B | | | | | | | | | |
| VWF)- | −0.0307054 | −0.3167743 | 0.05587034 | 0.12035451 | −0.1420664 | −0.0016475 | −0.1274129 | −0.107857 | −0.0200936 |
| ITGB3 | | | | | | | | | |
| VWF)- | −0.1508054 | −0.0349167 | 0.12448926 | 0.12742967 | −0.125 | −0.0513294 | −0.0553542 | 0.15053197 | −0.0163373 |
| STAB2 | | | | | | | | | |
| VWF)- | −0.1941955 | −0.3157097 | −0.0437189 | 0.27397622 | −0.0212857 | −0.0908672 | 0.03057157 | −0.0897292 | 0.21687144 |
| SELP | | | | | | | | | |
| VWF)- | −0.0518184 | 0.05718427 | −0.0835091 | 0.19002438 | 0.08810152 | −0.004942 | −0.1761023 | 0.12919165 | 0.09499341 |
| SIRPA | | | | | | | | | |
| VWF)- | 0.00546826 | −0.1831478 | 0.08189215 | −0.130197 | 0.27453629 | 0.17256944 | 0.05362495 | 0.16515696 | −0.1039526 |
| TNFRSF11B | | | | | | | | | |
| VWF)- | −0.300896 | −0.318071 | −0.0845071 | 0.04974633 | −0.0062679 | 0.10852662 | 0.20119415 | −0.1859148 | 0.11370224 |
| GP1BA | | | | | | | | | |
| CDH1)- | −0.0565236 | −0.0179183 | −0.0533131 | −0.0675889 | 0.05146793 | 0.0179189 | −0.0189139 | 0.05685487 | −0.0824797 |
| EGFR | | | | | | | | | |
| CDH1)- | 0.34312821 | −0.2173985 | 0.18129117 | −0.2345268 | 0.17233202 | 0.4231124 | −0.1848546 | −0.5040356 | 0.43810528 |
| LRP5 | | | | | | | | | |
| CDH1)- | 0.00237154 | 0.07206851 | −0.0320859 | 0.00197628 | −0.0555409 | 0.09762846 | 0.09540438 | −0.2159491 | 0.01956586 |
| IGF1R | | | | | | | | | |
| CDH1)- | 0.30152508 | 0.14394414 | −0.198511 | 0.15646102 | −0.0799842 | 0.24612141 | 0.15002471 | −0.358324 | −0.1332762 |
| ITGB7 | | | | | | | | | |
| CDH1)- | 0.32898551 | −0.3030303 | 0.23228697 | −0.2741765 | 0.30831055 | 0.47167325 | 0.06456288 | −0.2351856 | 0.08682763 |
| ERBB3 | | | | | | | | | |
| CDH1)- | 0.37075099 | 0.28162983 | −0.1844501 | 0.21397279 | −0.0426009 | 0.18050066 | 0.06070501 | −0.3147666 | −0.3571382 |
| KLRG1 | | | | | | | | | |
| CDH1)- | −0.1241188 | −0.0703557 | 0.03557781 | 0.03162055 | 0.08920807 | 0.02055471 | 0.09283479 | −0.1865138 | 0.07253203 |
| CDH2 | | | | | | | | | |
| CDH1)- | −0.0920949 | 0.03346509 | −0.2699532 | −0.0521739 | −0.0212878 | −0.1902503 | −0.0006261 | 0.23327514 | −0.0302382 |
| ITGAE | | | | | | | | | |
| CDH1)- | −0.0960569 | −0.0023719 | 0.16143645 | 0.06456714 | 0.05867416 | −0.1148994 | −0.0223748 | 0.04236395 | 0.08532367 |
| PTPRM | | | | | | | | | |
| CDH1)- | 0.3629776 | −0.5492754 | 0.32898551 | −0.5645586 | 0.37931489 | 0.56864295 | −0.3527784 | −0.488356 | 0.6765045 |
| PTPRF | | | | | | | | | |
| SEMA7A)- | −0.1533951 | 0.51797188 | 0.46486345 | 0.41569222 | 0.38558582 | −0.324319 | −0.0305665 | 0.58511199 | −0.0328063 |
| ITGB1 | | | | | | | | | |
| SEMA7A)- | −0.1473428 | 0.1393602 | 0.29464874 | −0.0171942 | −0.0589708 | −0.291992 | 0.04849284 | −0.2148363 | 0.17496706 |
| ITGA1 | | | | | | | | | |
| RIMS2)- | 0.06858255 | −0.1042051 | −0.2593094 | 0.08802504 | 0.16604027 | −0.0846017 | −0.0803745 | 0.0030306 | −0.1139733 |
| ABCA1 | | | | | | | | | |
| ANOS1)- | −0.0213579 | 0.02399473 | 0.06407384 | −0.0459816 | −0.0291173 | 0.13056653 | 0.0859025 | −0.2268775 | 0.14413702 |
| SDC2 | | | | | | | | | |
| ANOS1)- | −0.1942119 | 0.35042855 | 0.32225743 | −0.3096179 | −0.397022 | 0.30950656 | 0.17367242 | −0.3228803 | 0.24492754 |
| FGFR1 | | | | | | | | | |
| TIMP2)- | 0.22516469 | 0.24189723 | −0.2654809 | 0.18484848 | 0.20658762 | 0.02147563 | −0.2036891 | 0.26785244 | 0.15599473 |
| CD44 | | | | | | | | | |
| TIMP2)- | −0.2306983 | 0.48880105 | 0.56600791 | −0.4115942 | −0.3841897 | 0.71870883 | 0.67457181 | −0.2134387 | 0.63438735 |
| ITGB1 | | | | | | | | | |
| LGALS3BP)- | 0.06403162 | 0.29617918 | 0.31620553 | 0.37681159 | 0.30329381 | 0.05401845 | −0.2546772 | −0.0450593 | −0.355863 |
| ITGB1 | | | | | | | | | |
| IL1A)- | −0.0908462 | 0.09682547 | 0.03521806 | 0.06851346 | −0.0264216 | 0.20988469 | −0.0948773 | 0.44749763 | 0.185639 |
| IL1R1 | | | | | | | | | |
| IL1A)- | 0.00712826 | −0.2655275 | 0.25431549 | 0.24361804 | −0.2437578 | 0.00204223 | −0.0456537 | 0.0743083 | −0.0677207 |
| IL1RAP | | | | | | | | | |
| IL1A)- | −0.0262029 | 0.22487006 | 0.15401396 | −0.0476959 | 0.09165486 | −0.0237821 | −0.1246623 | 0.05757576 | 0.03030303 |
| IL1R2 | | | | | | | | | |
| IL1B)- | −0.0091646 | −0.0033621 | −0.1490589 | 0.06778879 | −0.0569866 | 0.02701367 | −0.0014494 | −0.02563 | −0.0412398 |
| SIGIRR | | | | | | | | | |
| IL1B)- | 0.2745887 | 0.31320464 | 0.23242017 | −0.2136434 | −0.0246426 | −0.1049094 | 0.03498715 | −0.1277101 | −0.1815607 |
| IL1R1 | | | | | | | | | |
| IL1B)- | 0.01137263 | 0.12453032 | −0.0724595 | 0.15455055 | 0.2464691 | 0.03643311 | 0.31940653 | −0.1307112 | 0.04189861 |
| ADRB2 | | | | | | | | | |
| IL1B)- | 0.02511702 | 0.29296599 | 0.04374198 | −0.2411147 | 0.21582449 | 0.01343918 | 0.32683312 | 0.03682598 | −0.1347212 |
| IL1RAP | | | | | | | | | |
| IL1B)- | 0.10356651 | 0.00857011 | 0.10236715 | −0.151059 | 0.00797285 | −0.1760928 | 0.18001516 | −0.150532 | −0.1747752 |
| IL1R2 | | | | | | | | | |
| LCN1)- | −0.0927995 | −0.2834734 | 0.11839243 | 0.35089106 | −0.0024408 | −0.1918503 | 0.05580296 | −0.0695652 | 0.26930171 |
| LMBR1L | | | | | | | | | |
| CNTN3)- | 0.04765043 | −0.1442504 | 0.03360902 | −0.0029654 | −0.121322 | 0.07434258 | 0.19776672 | −0.3133483 | 0.25783926 |
| PTPRG | | | | | | | | | |
| IL7)- IL7R | −0.222376 | −0.2393016 | 0.19769971 | 0.08194993 | −0.1551287 | 0.06528542 | 0.05654223 | −0.1081722 | −0.0238472 |
| IL7)- | 0.02747579 | −0.1171471 | 0.07372376 | 0.11620553 | −0.0342032 | 0.01284627 | 0.20831687 | 0.09888336 | −0.0909091 |
| IL2RG | | | | | | | | | |
| CKLF)- | −0.0640016 | −0.2562041 | 0.1894908 | −0.0403175 | −0.0343417 | 0.03847294 | −0.015109 | 0.31344906 | 0.38551994 |
| CCR4 | | | | | | | | | |
| FARP2)- | 0.01291726 | 0.01509161 | 0.03614538 | −0.1736495 | 0.08202021 | 0.19414342 | −0.3731787 | 0.08807932 | −0.0205534 |
| PLXNA3 | | | | | | | | | |
| FARP2)- | −0.0751335 | −0.2572822 | −0.1702976 | 0.14400527 | −0.0432906 | 0.15205218 | 0.32418543 | 0.03188616 | 0.52437418 |
| PLXNA1 | | | | | | | | | |
| FARP2)- | −0.1256096 | −0.0243846 | −0.1571975 | −0.1109391 | 0.13690868 | 0.37114625 | −0.0495454 | 0.06337286 | −0.1741164 |
| PLXNA2 | | | | | | | | | |
| FARP2)- | 0.07494562 | 0.23718204 | −0.1035926 | 0.02687747 | 0.12065103 | 0.10963959 | −0.0452688 | 0.0146274 | 0.18208169 |
| PLXNA4 | | | | | | | | | |
| LAMC2)- | 0.31304348 | −0.3205534 | 0.33096179 | −0.373913 | 0.4773386 | 0.47667984 | −0.1633729 | −0.168643 | 0.3342556 |
| ITGA6 | | | | | | | | | |
| LAMC2)- | −0.1 | −0.0048748 | −0.0324111 | −0.1289855 | 0.13847167 | 0.05059289 | −0.5968379 | −0.5361001 | 0.66982872 |
| CD151 | | | | | | | | | |
| LAMC2)- | −0.1511199 | 0.23504611 | 0.32911726 | 0.26824769 | 0.4030303 | −0.3429513 | −0.0264822 | 0.67720685 | 0.00171278 |
| ITGB1 | | | | | | | | | |
| LAMC2)- | 0.37641634 | −0.1196469 | 0.36824769 | −0.2325735 | 0.44426877 | 0.46916996 | −0.4635046 | −0.5981555 | 0.67255238 |
| ITGA2 | | | | | | | | | |
| LAMC2)- | 0.4599473 | −0.3247694 | 0.47957839 | −0.2638999 | 0.43517787 | 0.44848485 | 0.05823452 | −0.084058 | −0.1046113 |
| COL17A1 | | | | | | | | | |
| NID1)- | −0.2220758 | 0.10942389 | 0.21318225 | −0.1616601 | −0.1862978 | 0.44598155 | 0.1942029 | −0.2808959 | 0.13214756 |
| ITGB1 | | | | | | | | | |
| NID1)- | −0.0131778 | −0.0025035 | −0.0175248 | 0.05006917 | −0.0233876 | 0.08558722 | 0.05494433 | 0.16168671 | 0.14856051 |
| ITGB3 | | | | | | | | | |
| NID1)- | −0.0012518 | −0.3197734 | 0.00039541 | 0.12859025 | −0.0136413 | −0.0062586 | −0.1569739 | 0.19803676 | −0.1442688 |
| COL13A1 | | | | | | | | | |
| NID1)- | 0.18755558 | 0.02595606 | 0.15731743 | 0.26086957 | −0.3054018 | −0.2324111 | 0.27338603 | 0.27299078 | −0.0791831 |
| PTPRF | | | | | | | | | |
| NMU)- | 0.26472434 | −0.140729 | −0.1316414 | −0.2298495 | −0.1952103 | 0.0563937 | 0.14790171 | 0.07997628 | −0.0566535 |
| ADRA2A | | | | | | | | | |
| NMU)- | 0.03322566 | −0.0383627 | −0.3058048 | −0.0479594 | 0.03289713 | 0.21887663 | −0.1342211 | −0.0325471 | 0.17654809 |
| NMUR1 | | | | | | | | | |
| FGF2)- | −0.3631751 | −0.0469401 | −0.3722858 | 0.29934124 | −0.1237154 | −0.2196311 | −0.2645586 | −0.1201581 | −0.0462451 |
| SDC1 | | | | | | | | | |
| FGF2)- | −0.0968575 | −0.0374332 | −0.159789 | 0.04492754 | 0.14389248 | 0.08748929 | −0.272895 | 0.10264181 | −0.2209486 |
| FGFR1 | | | | | | | | | |
| FGF2)- | −0.0133369 | 0.19211771 | −0.1929663 | 0.26350461 | −0.0251319 | 0.05533961 | −0.1298154 | 0.1418407 | 0.31910408 |
| FGFR2 | | | | | | | | | |
| FGF2)- | −0.4737583 | 0.31715927 | −0.559254 | 0.13570487 | −0.0644269 | −0.1894598 | −0.4 | −0.4413702 | 0.43280632 |
| CD44 | | | | | | | | | |
| FGF2)- | 0.09698313 | −0.3347866 | −0.2753703 | 0.05836627 | 0.54439991 | 0.01330698 | −0.1047531 | 0.28102767 | −0.2108037 |
| SDC3 | | | | | | | | | |
| FGF2)- | 0.0089787 | −0.189807 | −0.0253516 | −0.2959157 | −0.0119895 | 0.10698287 | −0.1177866 | −0.0057971 | 0.01462451 |
| SDC4 | | | | | | | | | |
| FGF2)- | −0.0341323 | −0.0285866 | −0.0627849 | −0.128722 | −0.0797101 | 0.08445323 | 0.13886693 | 0.16047431 | 0.13386034 |
| SDC2 | | | | | | | | | |
| FGF2)- | 0.01439232 | −0.3031631 | −0.2383598 | −0.2795784 | 0.07802821 | −0.1212121 | −0.3341901 | −0.0155468 | −0.1222661 |
| FGFRL1 | | | | | | | | | |
| FGF2)- | 0.0849043 | −0.3060679 | 0.34277065 | 0.27259552 | −0.0561691 | −0.2693765 | 0.05102681 | 0.03583781 | −0.054809 |
| FGFR4 | | | | | | | | | |
| FGF2)- | 0.19898377 | −0.4783294 | 0.13840948 | 0.11159788 | −0.1821237 | −0.0220026 | 0.28840643 | 0.13109354 | −0.424454 |
| FGFR3 | | | | | | | | | |
| FGF2)- | 0.4012686 | −0.0368391 | −0.0307043 | 0.0284585 | 0.12162741 | 0.32963268 | −0.2189425 | 0.38333059 | −0.272859 |
| NRP1 | | | | | | | | | |
| IL11)- | 0.09540916 | 0.12862511 | 0.12657394 | 0.2413441 | −0.0715015 | 0.16103314 | −0.270427 | 0.06552031 | 0.16845642 |
| IL11RA | | | | | | | | | |
| IL11)- | 0.05477015 | 0.27167576 | 0.33648621 | −0.3208591 | −0.2595375 | 0.00790618 | 0.09394868 | −0.144537 | −0.067657 |
| IL6ST | | | | | | | | | |
| WNT5A)- | −0.1163366 | 0.13373321 | 0.14401531 | −0.1694558 | −0.1985769 | 0.27843848 | 0.2314888 | −0.1341941 | 0.2229249 |
| PTK7 | | | | | | | | | |
| WNT5A)- | 0.05707884 | −0.1793436 | 0.14790405 | −0.0284622 | −0.1085782 | 0.02898933 | 0.02898551 | 0.00685112 | 0.24993412 |
| LDLR | | | | | | | | | |
| WNT5A)- | −0.0078444 | 0.00088983 | 0.07052466 | 0.00046121 | −0.1796021 | −0.0205588 | 0.05533597 | −0.1002767 | −0.0716097 |
| LRP5 | | | | | | | | | |
| WNT5A)- | −0.1303889 | −0.1829747 | 0.10408359 | 0.19239005 | −0.1696702 | −0.178835 | 0.08347333 | 0.19172487 | −0.0143615 |
| FZD5 | | | | | | | | | |
| WNT5A)- | −0.230498 | 0.07942263 | 0.23367506 | 0.23771248 | 0.15269037 | −0.0263548 | −0.3886272 | 0.23307751 | 0.01805007 |
| ROR1 | | | | | | | | | |
| WNT5A)- | −0.0504894 | 0.05615608 | −0.0725799 | −0.0732639 | 0.09456031 | 0.17400758 | −0.0901993 | 0.10804045 | −0.1272727 |
| VANGL2 | | | | | | | | | |
| WNT5A)- | −0.0833141 | −0.1649091 | −0.0631138 | 0.21083147 | 0.06681162 | −0.1891616 | 0.1631201 | 0.07589183 | −0.1986825 |
| RYK | | | | | | | | | |
| WNT5A)- | −0.2105818 | 0.13538098 | 0.37024954 | 0.03808143 | −0.1946559 | 0.19863578 | −0.2735628 | 0.02820336 | −0.0516469 |
| ROR2 | | | | | | | | | |
| WNT5A)- | −0.1388084 | 0.11086213 | −0.0943844 | 0.05982343 | −0.0612729 | −0.1073923 | 0.07075099 | 0.21554677 | −0.0218709 |
| PTPRK | | | | | | | | | |
| WNT5A)- | 0.01443544 | −0.1041392 | −0.0383028 | 0.01159573 | 0.10410289 | −0.0591026 | 0.06481647 | −0.0745767 | −0.0104084 |
| ADRB2 | | | | | | | | | |
| WNT5A)- | −0.1352456 | 0.25441606 | 0.20054067 | −0.0093556 | 0.03974296 | 0.08760135 | −0.0081057 | −0.0637316 | −0.0175231 |
| FZD1 | | | | | | | | | |
| WNT5A)- | −0.0717111 | 0.03895334 | 0.12763853 | 0.14586902 | 0.00936305 | −0.1017262 | −0.0129219 | −0.0740448 | −0.2873518 |
| FZD4 | | | | | | | | | |
| WNT5A)- | −0.0140775 | 0.21862645 | 0.18512049 | 0.0395309 | −0.2726943 | 0.14520169 | −0.0551511 | −0.051206 | −0.1442688 |
| FZD9 | | | | | | | | | |
| WNT5A)- | 0.26220219 | −0.3942261 | −0.2214898 | 0.00527096 | −0.0852662 | 0.03419536 | 0.07444986 | 0.03201686 | −0.0290523 |
| FZD7 | | | | | | | | | |
| WNT5A)- | 0.01621622 | −0.0997232 | 0.22256651 | 0.11529846 | −0.0350588 | −0.067475 | −0.1062169 | −0.2535248 | −0.1198946 |
| FZD3 | | | | | | | | | |
| WNT5A)- | −0.0252439 | −0.0506855 | 0.00599967 | −0.1066017 | −0.2538636 | 0.11384899 | 0.28875491 | −0.0895916 | 0.22990777 |
| FZD8 | | | | | | | | | |
| WNT5A)- | −0.1599104 | 0.10796205 | 0.10776432 | −0.0002635 | −0.0057979 | 0.09053173 | 0.08063241 | 0.02457342 | 0.09920949 |
| ANTXR1 | | | | | | | | | |
| WNT5A)- | −0.1796664 | 0.02728711 | 0.04390244 | −0.0390038 | 0.05376911 | −0.2416793 | −0.125313 | 0.14332785 | −0.2445323 |
| FZD6 | | | | | | | | | |
| ICAM1)- | −0.3634986 | −0.0826882 | 0.17642794 | 0.21140354 | −0.302521 | 0.45278171 | 0.00850195 | 0.05968379 | 0.18642951 |
| ITGAM | | | | | | | | | |
| ICAM1)- | −0.0656893 | −0.1342118 | 0.03780862 | −0.0133074 | 0.18858973 | 0.09275668 | −0.1980094 | 0.21119895 | 0.29341238 |
| ITGAL | | | | | | | | | |
| ICAM1)- | −0.1349366 | −0.0118597 | 0.04007118 | 0.04183273 | 0.16154861 | 0.02226687 | −0.0756483 | 0.23465086 | 0.13596838 |
| ITGAX | | | | | | | | | |
| ICAM1)- | −0.0424971 | −0.0147587 | 0.00994894 | −0.0771435 | −0.0737178 | −0.0433479 | 0.25638999 | 0.23675889 | −0.3472991 |
| MUC1 | | | | | | | | | |
| ICAM1)- | −0.1612308 | 0.25280844 | 0.22320723 | −0.1393985 | −0.1057071 | −0.0134396 | −0.0620778 | 0.05645772 | −0.241502 |
| EGFR | | | | | | | | | |
| ICAM1)- | 0.27165212 | 0.14462198 | 0.20175265 | 0.17497283 | −0.0883128 | −0.1292533 | −0.1161435 | 0.08050066 | 0.1055336 |
| CAV1 | | | | | | | | | |
| ICAM1)- | 0.03175754 | −0.2730357 | 0.23709268 | 0.01712836 | 0.07346401 | −0.1590303 | −0.1042298 | −0.1769433 | 0.22661397 |
| IL2RA | | | | | | | | | |
| ICAM1)- | 0.07985505 | 0.0081041 | −0.0501186 | −0.0836655 | 0.16941223 | −0.0105405 | −0.1336277 | 0.24044796 | 0.25125165 |
| ITGB2 | | | | | | | | | |
| ICAM1)- | 0.06477119 | −0.1409982 | 0.17420317 | 0.03689186 | −0.0436936 | −0.0508614 | −0.1009803 | 0.25304697 | 0.16587615 |
| SPN | | | | | | | | | |
| ICAM1)- | −0.1739474 | −0.0599572 | 0.06386976 | −0.1189104 | 0.16027945 | −0.2407932 | −0.0740741 | −0.2048816 | −0.1841897 |
| IL2RG | | | | | | | | | |
| F13A1)- | 0.03584017 | 0.0737227 | 0.07533863 | −0.2578393 | 0.09411456 | −0.1916996 | 0.07131089 | −0.0790514 | −0.1552042 |
| ITGA4 | | | | | | | | | |
| F13A1)- | 0.34318279 | −0.2950884 | −0.2297328 | −0.0516469 | −0.0364954 | 0.13583663 | 0.02147563 | 0.02187088 | −0.0204216 |
| ITGB1 | | | | | | | | | |
| BST1)- | 0.01752421 | 0.17912906 | 0.01844715 | −0.0399223 | −0.0020752 | −0.0671959 | −0.0747719 | 0.09525692 | 0.03768116 |
| CAV1 | | | | | | | | | |
| KNG1)- | −0.0562638 | 0.26695655 | 0.05378506 | −0.1649593 | −0.0690394 | 0.08821107 | 0.0332169 | −0.0171278 | 0.05942029 |
| ITGAM | | | | | | | | | |
| KNG1)- | −0.3055739 | 0.09368515 | 0.02049086 | −0.0315557 | −0.0658168 | 0.41083075 | 0.19731208 | 0.04123983 | −0.0573123 |
| ADRA2A | | | | | | | | | |
| KNG1)- | 0.08834865 | 0.05817439 | −0.2135091 | −0.0900557 | 0.18875198 | −0.1383445 | 0.13494548 | 0.15046113 | 0.20750988 |
| PLAUR | | | | | | | | | |
| KNG1)- | 0.2067398 | 0.22017986 | −0.057397 | −0.0931519 | −0.0213166 | −0.0424915 | −0.0023721 | −0.0426877 | −0.0886693 |
| ITGB2 | | | | | | | | | |
| KNG1)- | −0.1114735 | −0.0357743 | 0.03399545 | 0.20718733 | 0.08089858 | −0.1594914 | 0.14782609 | −0.083004 | 0.12042161 |
| SDC2 | | | | | | | | | |
| KNG1)- | 0.27659364 | 0.20041506 | −0.0497875 | −0.1173293 | 0.22147541 | −0.3557334 | 0.22258831 | −0.1201001 | −0.1756258 |
| BDKRB2 | | | | | | | | | |
| KNG1)- | 0.0976116 | 0.05718615 | −0.2168164 | −0.1383445 | 0.34164552 | −0.1479066 | 0.00679443 | 0.11351209 | 0.28629776 |
| GP1BA | | | | | | | | | |
| VCAN)- | 0.24862479 | 0.24421094 | −0.2753666 | −0.0715415 | 0.06458842 | −0.0421607 | −0.3082449 | 0.34453228 | 0.33662714 |
| ITGA4 | | | | | | | | | |
| VCAN)- | 0.29500313 | 0.08063507 | −0.1339306 | −0.0822134 | 0.18906456 | 0.01251647 | −0.1545455 | 0.24426877 | 0.06245059 |
| CD44 | | | | | | | | | |
| VCAN)- | 0.33625404 | 0.02332093 | 0.14379861 | −0.0272727 | −0.1271212 | −0.3927007 | 0.03248872 | 0.27813828 | 0.01357049 |
| EGFR | | | | | | | | | |
| VCAN)- | −0.1382127 | 0.41055371 | 0.51029349 | −0.0714097 | −0.1156785 | 0.48326746 | 0.60500659 | −0.2146245 | 0.51607378 |
| ITGB1 | | | | | | | | | |
| VCAN)- | 0.12184597 | −0.0689087 | −0.0681721 | −0.06917 | 0.14293253 | −0.0240456 | −0.0458861 | 0.23050825 | −0.0151515 |
| TLR1 | | | | | | | | | |
| TNC)- | −0.4254506 | 0.40500826 | 0.52519031 | −0.1471673 | −0.1692414 | 0.44876313 | 0.17102013 | −0.0020422 | 0.27259552 |
| ITGA5 | | | | | | | | | |
| TNC)- | −0.188402 | 0.16072489 | 0.10378913 | −0.0567852 | 0.31910408 | 0.22542819 | −0.0092227 | 0.19670619 | −0.1499341 |
| SDC1 | | | | | | | | | |
| TNC)- | 0.30867575 | −0.2125206 | 0.01822736 | −0.2372859 | 0.37279649 | 0.10916038 | −0.2915418 | −0.04875 | −0.0986825 |
| EGFR | | | | | | | | | |
| TNC)- | −0.0125227 | −0.2077567 | 0.00906155 | −0.0571184 | 0.02075236 | 0.38616373 | −0.2039001 | −0.0068522 | 0.16081428 |
| ITGB3 | | | | | | | | | |
| TNC)- | −0.0321582 | −0.210346 | 0.10662274 | 0.18656126 | −0.0960474 | −0.0740448 | 0.07259552 | 0.19578393 | 0.0370224 |
| ITGB6 | | | | | | | | | |
| TNC)- | 0.25601319 | −0.073542 | −0.0989942 | 0.10500659 | 0.33901445 | −0.1350461 | −0.2197462 | −0.0453228 | 0.30540184 |
| PTPRB | | | | | | | | | |
| TNC)- | 0.13638461 | 0.10286995 | −0.0414275 | −0.0068513 | −0.228304 | 0.05678898 | 0.20898192 | −0.0126491 | 0.18419579 |
| ITGA8 | | | | | | | | | |
| TNC)- | −0.2620272 | −0.0516639 | 0.06437146 | 0.24216074 | 0.04397707 | 0.07813426 | 0.00745039 | −0.0374201 | 0.12490119 |
| ITGA7 | | | | | | | | | |
| TNC)- | −0.258056 | −0.0035585 | 0.110972 | −0.0679842 | −0.1650856 | 0.27865613 | 0.20289855 | −0.0333333 | 0.13794466 |
| ITGB1 | | | | | | | | | |
| TNC)- | 0.19775948 | −0.2399341 | 0.23387151 | −0.0396574 | 0.0599473 | 0.11936759 | −0.1075099 | −0.1938076 | 0.23689065 |
| SDC4 | | | | | | | | | |
| TNC)- | −0.1117628 | 0.31031303 | 0.24677037 | 0.20434783 | 0.25617712 | −0.3218709 | −0.1736839 | 0.21317523 | −0.0447958 |
| CNTN1 | | | | | | | | | |
| TNC)- | 0.01060956 | 0.0050748 | 0.12125207 | 0.32428515 | −0.2255599 | −0.2931489 | −0.0140975 | 0.11080369 | −0.217947 |
| ITGA2 | | | | | | | | | |
| SEMA3A)- | −0.2372178 | −0.1567516 | 0.15606766 | 0.02220099 | 0.02118546 | 0.13738346 | 0.21402215 | −0.2457007 | −0.0458498 |
| PLXNA4 | | | | | | | | | |
| SEMA3A)- | −0.2706988 | −0.0137108 | 0.12838529 | 0.23940182 | −0.1721228 | 0.17873378 | −0.1366124 | −0.1096874 | 0.04650856 |
| PLXNA3 | | | | | | | | | |
| SEMA3A)- | −0.1293385 | −0.2073762 | −0.0196809 | 0.04565368 | −0.2380074 | 0.01014593 | 0.08289131 | −0.0870281 | 0.33465086 |
| PLXNA1 | | | | | | | | | |
| SEMA3A)- | 0.26265826 | −0.0862859 | −0.1486023 | −0.053625 | 0.03439415 | 0.01126705 | −0.0463845 | 0.01831659 | −0.0957839 |
| NRP1 | | | | | | | | | |
| SEMA3A)- | −0.0017139 | 0.23447598 | 0.03718232 | 0.08795046 | −0.0505353 | 0.08056919 | −0.1793385 | 0.14571805 | −0.0967753 |
| PLXNA2 | | | | | | | | | |
| SEMA3A)- | −0.1178798 | 0.04363733 | 0.0237334 | −0.3098916 | −0.2814034 | 0.02661923 | 0.11872447 | 0.30564981 | 0.21304348 |
| NRP2 | | | | | | | | | |
| WNT2)- | −0.0341872 | −0.0405971 | −0.0466443 | −0.3341238 | −0.3061716 | 0.40347991 | 0.16673258 | −0.2257374 | 0.20152179 |
| FZD1 | | | | | | | | | |
| WNT2)- | −0.0915412 | −0.2833229 | 0.15763613 | 0.09328063 | −0.0044831 | 0.08550725 | 0.00293391 | −0.1960539 | −0.0542179 |
| FZD4 | | | | | | | | | |
| WNT2)- | −0.035042 | 0.13055657 | 0.00830587 | −0.1591568 | 0.14074392 | 0.19322527 | 0.07587639 | −0.124757 | −0.0125169 |
| FZD9 | | | | | | | | | |
| WNT2)- | −0.0811969 | −0.0450471 | 0.14533831 | 0.31621595 | 0.11832916 | 0.12931915 | −0.0617361 | −0.0451281 | −0.1816325 |
| FZD7 | | | | | | | | | |
| WNT2)- | −0.0431731 | −0.0797443 | −0.0020435 | 0.11607378 | 0.04553092 | −0.1719594 | 0.18460069 | 0.03070334 | 0.04413848 |
| FZD3 | | | | | | | | | |
| WNT2)- | −0.0688701 | −0.0163443 | 0.19905729 | 0.19341238 | 0.05232118 | −0.0786561 | −0.3307084 | 0.06449488 | −0.0413716 |
| FZD8 | | | | | | | | | |
| WNT2)- | 0.13353987 | −0.2762473 | 0.32932377 | 0.24164169 | −0.3137332 | −0.1702464 | 0.1685334 | 0.0329435 | −0.1497793 |
| FZD5 | | | | | | | | | |
| NPTX2)- | −0.1883834 | −0.0971865 | −0.2737569 | 0.14065485 | −0.1046832 | 0.0034259 | −0.2034786 | 0.04591719 | 0.06587615 |
| NPTXR | | | | | | | | | |
| GAL)- | −0.2084131 | 0.19554976 | −0.0605282 | −0.1499341 | −0.0291192 | 0.16640864 | 0.00230582 | 0.07503541 | −0.0945982 |
| ADRA2A | | | | | | | | | |
| GAL)- | 0.07833571 | 0.04700842 | 0.05328059 | 0.1400527 | 0.05962774 | −0.1467914 | −0.2926701 | 0.22756622 | 0.12358366 |
| GALR2 | | | | | | | | | |
| ADAM28)- | 0.11951509 | 0.11793385 | −0.1511436 | −0.0621871 | 0.13346077 | −0.0801054 | 0.11032757 | −0.1395257 | −0.1437418 |
| ITGA4 | | | | | | | | | |
| TIMP3)- | 0.02977602 | 0.35006588 | −0.3277997 | −0.3660079 | 0.2345191 | 0.05889328 | −0.3616601 | −0.0555995 | 0.35876153 |
| CD44 | | | | | | | | | |
| TIMP3)- | 0.11238472 | −0.1002635 | 0.31422925 | 0.10645586 | −0.2615283 | 0.02964427 | 0.27905138 | 0.22779974 | −0.140975 |
| MET | | | | | | | | | |
| TIMP3)- | −0.1151591 | 0.10777339 | 0.00316477 | −0.0806324 | 0.16107344 | 0.21009289 | −0.0149008 | −0.044074 | −0.0524374 |
| AGTR2 | | | | | | | | | |
| LUM)- | −0.1812912 | 0.0770751 | 0.19881423 | −0.1500659 | −0.1948617 | 0.63491436 | 0.3256917 | −0.1955204 | 0.19222661 |
| ITGB1 | | | | | | | | | |
| FGF7)- | 0.03913817 | −0.1088417 | 0.00244096 | −0.0471673 | −0.0594987 | −0.065617 | −0.2144461 | 0.18598063 | −0.1379447 |
| FGFR2 | | | | | | | | | |
| FGF7)- | 0.20767584 | 0.14613256 | 0.19793628 | −0.5089592 | 0.37406478 | −0.0006588 | 0.00059334 | 0.37814157 | 0.12503294 |
| FGFR4 | | | | | | | | | |
| FGF7)- | −0.2890558 | 0.18118329 | 0.18318397 | −0.1421607 | −0.3204638 | −0.1015218 | 0.1698511 | −0.1732657 | 0.20434783 |
| FGFR1 | | | | | | | | | |
| FGF7)- | −0.1039663 | 0.08697085 | −0.2245873 | −0.3786027 | 0.21256549 | 0.01093544 | −0.1674299 | −0.0745718 | 0.16252182 |
| FGFR3 | | | | | | | | | |
| MFGE8)- | 0.03762148 | −0.0574478 | 0.24138613 | 0.26027803 | −0.1640532 | 0.15850163 | 0.16318598 | 0.09797398 | −0.0367613 |
| ITGB3 | | | | | | | | | |
| IGFBP4)- | −0.1729908 | 0.21264822 | 0.29323582 | −0.1549407 | −0.2494152 | 0.27694335 | 0.15274621 | −0.1658762 | 0.23873518 |
| FZD8 | | | | | | | | | |
| CGN)- | 0.47536232 | −0.3066078 | 0.42463768 | −0.3174122 | 0.54216074 | 0.65217391 | −0.4944664 | −0.4967062 | 0.55879834 |
| F11R | | | | | | | | | |
| CGN)- | −0.084456 | −0.3504611 | 0.06285413 | −0.4317523 | 0.09803663 | −0.1763563 | −0.0744499 | 0.23149643 | 0.42028986 |
| TGFBR2 | | | | | | | | | |
| CGN)- | 0.51977997 | −0.5031961 | 0.35630974 | −0.5592752 | 0.32566723 | 0.59837281 | −0.2110709 | −0.4980401 | 0.6349918 |
| OCLN | | | | | | | | | |
| LEFTY1)- | −0.0643078 | −0.052316 | 0.04662888 | −0.2098225 | 0.06757603 | −0.0030304 | −0.1383764 | 0.32819499 | 0.08761528 |
| ACVR1B | | | | | | | | | |
| REN)- | −0.0940959 | 0.10668161 | 0.03812568 | 0.05369083 | 0.07458655 | 0.01534965 | −0.192983 | 0.14822134 | 0.08366271 |
| ATP6AP2 | | | | | | | | | |
| CALM2)- | −0.194611 | 0.29975624 | −0.0547647 | 0.26035971 | 0.02721761 | −0.1968509 | 0.17497036 | −0.217208 | −0.338494 |
| KCNQ3 | | | | | | | | | |
| CALM2)- | −0.1281992 | −0.3677207 | −0.1190155 | −0.3060606 | −0.081716 | −0.1783985 | −0.1708788 | −0.0335321 | 0.32727273 |
| EGFR | | | | | | | | | |
| CALM2)- | −0.2329381 | 0.22555995 | 0.08471673 | 0.23320158 | 0.07602108 | −0.2022398 | −0.1009223 | 0.39433465 | −0.3072464 |
| MYLK | | | | | | | | | |
| CALM2)- | 0.28870805 | −0.1233202 | 0.13775157 | −0.173386 | 0.11476004 | 0.30854396 | 0.06798643 | 0.03380672 | −0.2318841 |
| INSR | | | | | | | | | |
| CALM2)- | −0.1154302 | 0.30468724 | −0.0357096 | 0.28255213 | −0.0265516 | −0.1154302 | −0.2135327 | −0.0081697 | −0.6121414 |
| GP6 | | | | | | | | | |
| CALM2)- | −0.0483531 | 0.17641634 | −0.3092351 | 0.2168643 | −0.357784 | −0.0463768 | 0.12691304 | −0.1944664 | −0.5758893 |
| SCN10A | | | | | | | | | |
| CALM2)- | −0.0104747 | 0.10777339 | −0.1010447 | 0.15902503 | −0.1536434 | −0.0104088 | 0.26859576 | 0.04763003 | −0.1544137 |
| PLPP6 | | | | | | | | | |
| CALM2)- | 0.23012056 | 0.11343874 | −0.3617077 | 0.06100132 | −0.3446436 | 0.16147309 | −0.1738042 | −0.2207655 | −0.3450593 |
| AQP6 | | | | | | | | | |
| CALM2)- | −0.0613327 | −0.0080369 | 0.047379 | −0.0251647 | 0.01910975 | 0.01462499 | −0.0642483 | −0.1077769 | −0.4405797 |
| AQP1 | | | | | | | | | |
| CALM2)- | −0.1009981 | 0.25217391 | 0.03993279 | 0.25085639 | 0.08065632 | −0.0851863 | −0.1196666 | −0.2804625 | −0.1562582 |
| SCN4A | | | | | | | | | |
| NXPH2)- | −0.2193981 | −0.3067186 | 0.28268901 | −0.0554787 | −0.2672073 | 0.31647613 | −0.2466052 | −0.1415818 | 0.16667765 |
| NRXN1 | | | | | | | | | |
| LRIG1)- | 0.22606692 | −0.2388453 | 0.21892204 | 0.08978037 | 0.06165964 | −0.1059364 | −0.0203631 | 0.11515531 | −0.2674572 |
| EGFR | | | | | | | | | |
| LRIG1)- | −0.0028999 | −0.0600409 | −0.1014302 | −0.2087608 | 0.19495355 | 0.15617424 | 0.15915679 | 0.12476943 | −0.0874835 |
| MET | | | | | | | | | |
| UCN2)- | −0.1629923 | 0.03861106 | 0.365128 | 0.11648439 | −0.2147399 | 0.40204964 | −0.2600231 | −0.0814524 | −0.0485869 |
| IL10RB | | | | | | | | | |
| UCN2)- | −0.2371273 | 0.02299456 | 0.0400725 | 0.08077215 | 0.1423864 | −0.0622591 | −0.0951926 | 0.16489344 | 0.29823117 |
| CRHR2 | | | | | | | | | |
| UCN2)- | 0.02437819 | 0.23594136 | 0.2591287 | 0.15943605 | −0.1622416 | 0.29278256 | 0.32017438 | 0.37708752 | 0.39685102 |
| CRHR1 | | | | | | | | | |
| SFRP2)- | 0.0716827 | −0.2322211 | 0.22986461 | −0.039 | −0.2137892 | −0.1656345 | 0.27196363 | 0.08960337 | −0.3523173 |
| FZD5 | | | | | | | | | |
| MEGF10)- | 0.01103716 | 0.10417422 | −0.0260485 | 0.13090454 | 0.09784726 | 0.00461179 | −0.1352278 | −0.1330786 | −0.0209493 |
| ABCA1 | | | | | | | | | |
| IL9)- | −0.2466557 | −0.0254357 | 0.02214971 | −0.0538403 | 0.07106833 | −0.19138 | −0.124687 | 0.21298462 | 0.22714097 |
| IL2RG | | | | | | | | | |
| FGF18)- | −0.0855137 | −0.078819 | 0.08651185 | −0.2230934 | 0.00171532 | −0.0204263 | 0.11279693 | 0.14559589 | −0.2454545 |
| FGFR2 | | | | | | | | | |
| FGF18)- | −0.1738557 | −0.1412943 | −0.1823578 | 0.16195026 | 0.11123568 | −0.0604863 | −0.0477964 | 0.15652689 | −0.0527009 |
| FGFR4 | | | | | | | | | |
| FGF18)- | 0.00767811 | 0.30090947 | 0.23899289 | −0.3099325 | −0.1748213 | −0.0161434 | 0.08248781 | −0.1107451 | 0.11844532 |
| FGFR1 | | | | | | | | | |
| FGF18)- | −0.1177672 | 0.08185324 | −0.1401734 | −0.0253014 | 0.21968499 | −0.2630209 | −0.0898758 | 0.01725955 | 0.22965183 |
| FGFR3 | | | | | | | | | |
| CRH)- | −0.0971302 | 0.17500742 | 0.18670391 | 0.28248625 | −0.0443565 | 0.24554469 | 0.18910303 | −0.0567231 | 0.0428195 |
| ADRB2 | | | | | | | | | |
| CRH)- | −0.0538991 | 0.14555399 | 0.2031111 | 0.31008927 | −0.4174108 | 0.50291512 | −0.1236903 | −0.0864295 | 0.08998682 |
| CRHR2 | | | | | | | | | |
| CRH)- | −0.1448292 | −0.1760617 | 0.28356291 | 0.29572779 | 0.05419742 | 0.11126849 | 0.23925148 | 0.04453228 | −0.0768116 |
| GPR84 | | | | | | | | | |
| CRH)- | −0.1134027 | 0.1256548 | 0.0307317 | 0.23604203 | 0.0504731 | 0.00902563 | 0.07911917 | 0.14980731 | 0.02384717 |
| PTH1R | | | | | | | | | |
| CRH)- | 0.06675013 | −0.2173311 | 0.21583493 | 0.07234155 | −0.3150541 | −0.0062586 | −0.3439776 | 0.16884614 | −0.0305037 |
| VIPR1 | | | | | | | | | |
| CRH)- | −0.0081049 | −0.1290811 | −0.0072552 | 0.12516881 | −0.0075176 | 0.02615456 | −0.1594462 | 0.22629204 | 0.06758893 |
| MC2R | | | | | | | | | |
| CRH)- | −0.1032517 | −0.1060192 | 0.17001493 | 0.30000988 | −0.0210415 | 0.13274482 | 0.01925996 | −0.1100132 | 0.22371542 |
| CRHR1 | | | | | | | | | |
| CRH)- | −0.1155304 | 0.05024381 | −0.2132309 | 0.20940115 | 0.05249803 | 0.11511218 | −0.0920745 | 0.08117546 | 0.38328008 |
| ADRB3 | | | | | | | | | |
| LIN7C)- | −0.1388285 | 0.10749465 | 0.34962093 | 0.08999275 | 0.26578323 | −0.0252331 | −0.1378652 | 0.09210093 | 0.04532429 |
| ABCA1 | | | | | | | | | |
| SERPING1)- | −0.0664119 | 0.12194078 | 0.1904889 | −0.001647 | 0.17096511 | 0.07102385 | 0.13237923 | −0.0722098 | 0.10263843 |
| SELE | | | | | | | | | |
| IL18)- | −0.1606272 | −0.2510212 | 0.01832625 | 0.07720685 | −0.1202914 | 0.17378129 | 0.10473932 | −0.2477684 | −0.1538259 |
| CD48 | | | | | | | | | |
| IL18)- | 0.09858644 | 0.17578074 | 0.21281925 | 0.17694335 | 0.16073018 | 0.12842224 | 0.05799394 | −0.2598511 | −0.1397279 |
| IL18R1 | | | | | | | | | |
| IL18)- | −0.13647 | −0.081829 | 0.33781469 | −0.1291173 | 0.23253823 | −0.109043 | −0.1722513 | −0.0495487 | 0.09473303 |
| IL18BP | | | | | | | | | |
| QDPR)- | −0.050824 | −0.0516293 | 0.26775885 | −0.1597931 | −0.0639336 | 0.16103735 | −0.0601107 | 0.02191321 | −0.0064563 |
| DYSF | | | | | | | | | |
| PIGF)- | 0.08799104 | 0.04751549 | 0.02756165 | 0.03346839 | −0.1040177 | 0.15998419 | −0.1502768 | 0.04295691 | 0.11040843 |
| FLT1 | | | | | | | | | |
| PTH)- | −0.0049449 | 0.12361555 | 0.30237494 | −0.1639765 | −0.1363084 | 0.14145474 | 0.08032753 | −0.0280651 | 0.05599473 |
| ADRB2 | | | | | | | | | |
| PTH)- | 0.06263188 | 0.05392934 | −0.1389429 | 0.24612952 | −0.0231294 | 0.20390013 | −0.1821896 | 0.29525692 | 0.40434783 |
| GPR84 | | | | | | | | | |
| PTH)- | −0.1236196 | −0.1236815 | 0.04407786 | 0.07306147 | −0.2307794 | 0.27120598 | −0.1128767 | 0.07622122 | 0.06403162 |
| PTH1R | | | | | | | | | |
| PTH)- | 0.10845561 | 0.24544232 | −0.1682992 | 0.10245429 | −0.0564545 | −0.003953 | 0.2379305 | 0.1531671 | 0.12221234 |
| VIPR1 | | | | | | | | | |
| PTH)- | −0.1064293 | 0.14211311 | 0.22657565 | 0.04335079 | 0.02900175 | 0.15221402 | −0.0444225 | −0.0272122 | 0.09578708 |
| ADRB3 | | | | | | | | | |
| BMP3)- | −0.0435556 | 0.06938359 | −0.0256655 | 0.16938433 | 0.12646594 | −0.0594611 | −0.1359773 | 0.15000988 | −0.1243124 |
| BMPR2 | | | | | | | | | |
| BMP3)- | 0.0924486 | 0.04249992 | −0.1172282 | −0.0094212 | −0.2075285 | 0.03360127 | −0.1322677 | 0.05636076 | 0.25402681 |
| BMPR1A | | | | | | | | | |
| BMP3)- | −0.0815183 | −0.3817745 | 0.05027694 | 0.26603419 | −0.0153613 | 0.24570883 | −0.1678423 | 0.046648 | 0.29941698 |
| BMPR1B | | | | | | | | | |
| BMP6)- | 0.10071849 | −0.1843589 | −0.270648 | −0.0126499 | 0.14877286 | −0.0580465 | 0.02391462 | 0.0824165 | −0.1160117 |
| BMPR2 | | | | | | | | | |
| BMP6)- | 0.08885375 | 0.21771087 | 0.17876929 | −0.1135196 | 0.01315226 | −0.0591995 | 0.07013844 | −0.1930298 | 0.14730393 |
| BMPR1A | | | | | | | | | |
| BMP6)- | −0.0976301 | −0.1853476 | −0.1459007 | −0.0596258 | 0.13492667 | 0.0060622 | 0.10508276 | −0.0549498 | 0.10817221 |
| BMPR1B | | | | | | | | | |
| SEMA3D)- | 0.17443164 | 0.04197694 | −0.1289377 | −0.0330698 | 0.14737466 | −0.2102767 | 0.00362355 | 0.19697619 | 0.2002701 |
| PLXND1 | | | | | | | | | |
| APP)- | 0.00513851 | 0.04855232 | 0.03544487 | 0.11633729 | 0.11667435 | 0.15731225 | −0.0408459 | −0.1171278 | 0.10210804 |
| LRP10 | | | | | | | | | |
| APP)- PLD1 | −0.0565895 | −0.0041506 | 0.02345809 | −0.0648264 | −0.0924456 | 0.0654809 | 0.00092248 | 0.03939394 | −0.2760393 |
| APP)- | 0.12227017 | −0.0696334 | 0.10171613 | −0.0878788 | 0.21238472 | 0.22687747 | 0.13188406 | 0.12476943 | −0.0833992 |
| TSPAN15 | | | | | | | | | |
| APP)- | 0.07457426 | −0.0634408 | 0.21852559 | −0.2782609 | −0.0959847 | 0.48194993 | 0.36127672 | 0.03886693 | 0.25111989 |
| CAV1 | | | | | | | | | |
| APP)- | 0.05230911 | 0.12451003 | −0.1169379 | −0.1754941 | 0.27932409 | 0.13841036 | −0.2612075 | −0.0384071 | 0.19275362 |
| RPSA | | | | | | | | | |
| APP)- | 0.23775487 | 0.004875 | 0.15975493 | −0.1022398 | 0.1002635 | 0.19525692 | −0.0438735 | −0.0094862 | 0.11014493 |
| TNFRSF21 | | | | | | | | | |
| APP)- | −0.0491452 | 0.23755723 | −0.1351253 | 0.1654809 | 0.08603992 | −0.0494071 | −0.2380262 | −0.0267457 | 0.07839262 |
| NCSTN | | | | | | | | | |
| APP)- | 0.0235891 | 0.21200343 | −0.0551454 | 0.39072433 | 0.0366525 | −0.1912104 | −0.2107519 | 0.19213284 | −0.317797 |
| AGER | | | | | | | | | |
| WNT7A)- | 0.29232292 | −0.0862603 | 0.15169688 | −0.0803057 | −0.0656807 | 0.01462499 | −0.1499341 | −0.2305665 | 0.19565217 |
| LDLR | | | | | | | | | |
| WNT7A)- | 0.10102808 | −0.1589456 | −0.0531659 | 0.28268388 | −0.0059312 | 0.00546826 | 0.0034268 | −0.2343369 | −0.0167325 |
| RECK | | | | | | | | | |
| WNT7A)- | −0.0750873 | 0.01522241 | −0.0329895 | −0.0273395 | −0.0595018 | −0.0602366 | 0.05805028 | −0.0388164 | 0.14808959 |
| FZD9 | | | | | | | | | |
| WNT7A)- | 0.1178409 | −0.0030973 | 0.05977527 | 0.01831478 | −0.2809659 | 0.2727722 | 0.01831538 | −0.3267888 | −0.1697684 |
| FZD5 | | | | | | | | | |
| DKK2)- | 0.01061375 | −0.0078769 | 0.05352196 | 0.24211081 | −0.1025709 | 0.38736246 | −0.1970272 | 0.24457256 | 0.30442373 |
| KREMEN2 | | | | | | | | | |
| CXCL13)- | −0.0810704 | −0.1439447 | −0.1687712 | 0.09565532 | −0.0239908 | 0.23904737 | −0.0724313 | 0.3564676 | 0.15652174 |
| CXCR5 | | | | | | | | | |
| GDF6)- | −0.1742814 | 0.00654029 | −0.0208437 | −0.0640422 | −0.2536733 | 0.0560058 | 0.08491996 | −0.061203 | 0.25501499 |
| BMPR2 | | | | | | | | | |
| GDF6)- | −0.1235099 | −0.0099756 | 0.07978866 | 0.22210509 | −0.0504022 | 0.20876985 | −0.3366513 | 0.23782858 | 0.17154715 |
| BMPR1A | | | | | | | | | |
| GDF6)- | −0.2644873 | 0.00085883 | 0.24622408 | 0.18698732 | −0.0697567 | 0.23867418 | −0.1028413 | 0.12604184 | 0.38828683 |
| BMPR1B | | | | | | | | | |
| SST)- | 0.0178595 | −0.1507644 | −0.0456838 | −0.0401212 | −0.1803263 | 0.19529551 | 0.26500151 | −0.2033865 | 0.24545455 |
| SSTR5 | | | | | | | | | |
| WNT4)- | −0.0945038 | 0.38005802 | 0.02432513 | 0.0459861 | 0.01713457 | 0.02865896 | −0.0785477 | 0.03162055 | 0.0428195 |
| FZD8 | | | | | | | | | |
| WNT4)- | 0.28373657 | 0.06939502 | 0.0096889 | −0.0256284 | 0.08467038 | 0.01667381 | −0.1091053 | 0.14280067 | 0.03293808 |
| FZD6 | | | | | | | | | |
| ACE)- | −0.1699674 | 0.0014498 | −0.0461092 | −0.1173293 | −0.0016144 | 0.24617057 | 0.13356616 | −0.1388761 | −0.0490119 |
| BDKRB2 | | | | | | | | | |
| ACE)- | −0.2902429 | −0.1164454 | 0.20235467 | 0.01409796 | −0.0489896 | 0.06186382 | 0.02940596 | 0.20390013 | −0.0681159 |
| AGTR2 | | | | | | | | | |
| CALM3)- | 0.24215173 | −0.0480285 | −0.1280852 | −0.1822254 | −0.0612231 | 0.03755188 | 0.14768684 | −0.2405297 | 0.01712893 |
| KCNQ3 | | | | | | | | | |
| CALM3)- | 0.13944447 | 0.07714352 | −0.0983807 | −0.0306983 | −0.1574436 | −0.0265568 | −0.0309347 | −0.2237891 | −0.087747 |
| ESR1 | | | | | | | | | |
| CALM3)- | 0.05158273 | −0.2228005 | −0.2739879 | −0.3664032 | −0.2649539 | 0.47681159 | 0.11818182 | −0.2337286 | 0.20065876 |
| MYLK | | | | | | | | | |
| CALM3)- | −0.0628707 | 0.27115518 | −0.1882206 | 0.42318841 | −0.2566619 | 0.07881644 | 0.28031226 | 0.08922865 | −0.4073781 |
| INSR | | | | | | | | | |
| CALM3)- | −0.1121397 | 0.06861453 | 0.17163565 | 0.02305741 | −0.0927658 | 0.06759784 | −0.0530373 | −0.0697062 | −0.0521756 |
| GP6 | | | | | | | | | |
| CALM3)- | 0.22872954 | 0.27741362 | 0.04660448 | 0.07457181 | 0.04248025 | 0.17944664 | −0.0425462 | −0.3251647 | −0.1608696 |
| SCN10A | | | | | | | | | |
| CALM3)- | 0.09111572 | 0.13478705 | 0.20912535 | 0.08247694 | −0.0780735 | −0.1899335 | −0.1236658 | 0.08834574 | 0.00039526 |
| AQP6 | | | | | | | | | |
| CALM3)- | 0.37255419 | 0.32089331 | −0.0096214 | 0.05783926 | −0.0669522 | 0.44204355 | 0.18899507 | −0.3815672 | 0.04361001 |
| AR | | | | | | | | | |
| CALM3)- | 0.0671981 | 0.38136961 | 0.03907743 | 0.26060606 | −0.0108728 | −0.2721434 | −0.111166 | 0.07048981 | −0.2098814 |
| AQP1 | | | | | | | | | |
| CALM3)- | 0.45892081 | 0.13412827 | −0.032883 | 0.09855072 | −0.1949853 | 0.16134664 | 0.10273138 | −0.2481141 | 0.03992095 |
| SCN4A | | | | | | | | | |
| TFF2)- | 0.35153831 | −0.5333509 | 0.42672201 | −0.4538358 | 0.18559146 | 0.3613545 | −0.3130085 | −0.3891561 | 0.6403373 |
| MUC6 | | | | | | | | | |
| TFF1)- | 0.7828722 | −0.6667545 | 0.78665305 | −0.6409302 | 0.77005171 | 0.79631094 | −0.7029876 | −0.6955204 | 0.8366547 |
| MUC5AC | | | | | | | | | |
| S100B)- | −0.3251277 | −0.054739 | 0.28432052 | −0.1189182 | −0.0769307 | 0.05013506 | 0.0882915 | −0.1486166 | 0.22497447 |
| ALCAM | | | | | | | | | |
| S100B)- | 0.19687532 | −0.1120156 | 0.05398364 | 0.29301314 | −0.1712431 | −0.0224697 | 0.01773296 | 0.12571655 | 0.37945914 |
| AGER | | | | | | | | | |
| S100A1)- | 0.16899506 | −0.1811472 | −0.1081054 | −0.0575156 | 0.2011464 | 0.12870602 | 0.10731932 | 0.05020094 | 0.43170065 |
| TRPM3 | | | | | | | | | |
| S100A1)- | −0.1214458 | −0.2193667 | 0.06654138 | 0.14296538 | −0.1859691 | 0.20318213 | 0.1196638 | −0.0996739 | 0.07576007 |
| RYR1 | | | | | | | | | |
| S100A1)- | 0.01706642 | −0.3134555 | −0.0872603 | 0.10059951 | −0.1174222 | 0.21325516 | −0.2665876 | 0.15256917 | 0.30382082 |
| TLR4 | | | | | | | | | |
| S100A1)- | −0.0834377 | −0.1093209 | −0.1055686 | −0.062325 | −0.1479052 | 0.10592383 | 0.01957876 | 0.22738355 | 0.30712474 |
| AGER | | | | | | | | | |
| SCGB3A1)- | 0.111459 | −0.1102653 | −0.1989387 | 0.10138674 | 0.1845495 | 0.09223573 | 0.10489211 | 0.22288105 | 0.0252314 |
| MARCO | | | | | | | | | |
| CXCL16)- | 0.03004249 | −0.0625906 | 0.10032312 | 0.08590533 | 0.16363942 | −0.05639 | −0.106214 | −0.1616601 | −0.1091604 |
| CXCR6 | | | | | | | | | |
| TNFSF12)- | 0.10295483 | −0.1586863 | −0.04286 | 0.09961131 | 0.03533057 | 0.00876211 | −0.0587945 | 0.03260977 | 0.17965019 |
| TNFRSF25 | | | | | | | | | |
| TNFSF12)- | −0.0055402 | 0.08818104 | 0.0727573 | −0.0156796 | −0.3021019 | −0.1704987 | 0.18744853 | 0.04334794 | −0.2340657 |
| TNFRSF12A | | | | | | | | | |
| FGF11)- | −0.0102162 | −0.1425559 | 0.10351089 | −0.0134387 | −0.2151717 | −0.0737203 | −0.0452507 | −0.1832795 | −0.1919631 |
| FGFR2 | | | | | | | | | |
| FGF11)- | −0.2994991 | 0.24734729 | −0.0192472 | −0.0801054 | −0.267756 | 0.08808222 | 0.06324944 | −0.232624 | 0.19802372 |
| FGFR1 | | | | | | | | | |
| FGF11)- | −0.038819 | −0.0107431 | −0.0312799 | −0.102309 | −0.1487827 | 0.13530962 | −0.3592396 | 0.01923584 | 0.26430383 |
| FGFR3 | | | | | | | | | |
| FGF19)- | −0.1474697 | −0.2302168 | 0.20977119 | 0.06060606 | −0.2674145 | −0.0184465 | −0.1380608 | 0.11239212 | −0.2156785 |
| FGFR2 | | | | | | | | | |
| FGF19)- | −0.0901423 | 0.02866179 | −0.0085008 | −0.1791831 | −0.2205824 | 0.37874695 | 0.08604559 | −0.1699717 | 0.08893281 |
| FGFR1 | | | | | | | | | |
| FGF19)- | 0.21176781 | −0.0656278 | −0.0205951 | 0.13142725 | 0.00599611 | 0.0944664 | −0.049748 | −0.1945982 | 0.32906222 |
| FGFR3 | | | | | | | | | |
| VCAM1)- | 0.40191073 | 0.34722451 | −0.2485745 | −0.270751 | 0.25380613 | −0.3507246 | −0.2709418 | 0.443083 | 0.37694335 |
| ITGA4 | | | | | | | | | |
| VCAM1)- | 0.08301763 | −0.0535661 | −0.0450008 | 0.03465086 | 0.0342556 | 0.18761528 | −0.1649539 | 0.18418972 | −0.1461133 |
| ITGB1 | | | | | | | | | |
| VCAM1)- | 0.26078076 | 0.22289574 | −0.2600501 | 0.10197628 | 0.03070537 | 0.171278 | −0.1435772 | 0.1743083 | 0.17193676 |
| ITGB2 | | | | | | | | | |
| VCAM1)- | −0.0973151 | −0.0257618 | −0.1707132 | −0.030303 | 0.2826087 | 0.10869565 | −0.0009223 | 0.10329381 | −0.2204216 |
| EZR | | | | | | | | | |
| VCAM1)- | 0.24048757 | 0.11286444 | −0.2455516 | 0.10171278 | 0.32715054 | −0.1511199 | −0.331104 | 0.37509881 | 0.14532279 |
| MSN | | | | | | | | | |
| ARPC5)- | −0.2776295 | 0.02351856 | 0.06052816 | 0.09130435 | 0.30929731 | −0.3840833 | −0.1800604 | −0.0173266 | 0.02674572 |
| ADRB2 | | | | | | | | | |
| ARPC5)- | 0.17062486 | −0.2335387 | 0.15692216 | 0.01607378 | 0.21673254 | 0.12832675 | −0.0089592 | −0.1234519 | 0.25401845 |
| LDLR | | | | | | | | | |
| INHBB)- | 0.03069829 | −0.1401845 | 0.21621266 | −0.1981555 | 0.28044402 | 0.16245059 | 0.10632761 | 0.13043478 | −0.2256917 |
| SMAD3 | | | | | | | | | |
| INHBB)- | 0.06561265 | −0.1284585 | −0.0493542 | −0.097892 | −0.1217712 | 0.07351779 | −0.1433843 | −0.1006588 | 0.22463768 |
| ACVR1B | | | | | | | | | |
| PROK2)- | −0.0751181 | 0.1238368 | −0.1142829 | −0.0212808 | 0.03231659 | 0.04631551 | −0.3787261 | 0.18300395 | 0.22082414 |
| PROKR1 | | | | | | | | | |
| IHH)- | −0.327722 | −0.0166035 | −0.0832015 | 0.25798801 | −0.0782491 | −0.0012517 | −0.1713909 | 0.32570243 | −0.2551467 |
| PTCH1 | | | | | | | | | |
| IHH)- HHIP | −0.1881733 | 0.33877982 | 0.02527266 | 0.14704042 | −0.0839813 | −0.0164042 | 0.13118967 | 0.00408446 | 0.12055336 |
| IHH)- BOC | −0.2696033 | −0.0286599 | −0.0385477 | 0.10929214 | −0.0631197 | −0.0992917 | −0.0112004 | 0.01818421 | 0.02028986 |
| IHH)- | −0.1049649 | 0.07227566 | −0.2048891 | −0.150532 | −0.3061668 | 0.16082488 | 0.15950193 | −0.1108146 | 0.06798419 |
| PTCH2 | | | | | | | | | |
| CXCL3)- | 0.13824354 | 0.03203375 | −0.1806275 | −0.0342601 | −0.0278328 | 0.20218723 | 0.43186945 | −0.2585809 | −0.1763078 |
| CXCR2 | | | | | | | | | |
| CXCL5)- | −0.1145041 | −0.015154 | −0.3186362 | 0.11371722 | −0.1439785 | −0.1106133 | 0.37785279 | 0.04565518 | −0.1481091 |
| CXCR2 | | | | | | | | | |
| PPBP)- | −0.2098326 | 0.0161471 | 0.13779117 | −0.0376215 | −0.0484121 | 0.0871957 | −0.0068593 | 0.27729099 | −0.0287258 |
| CXCR2 | | | | | | | | | |
| PF4)- | −0.2527464 | −0.0518521 | 0.09294184 | 0.15752026 | −0.0781714 | 0.13463566 | −0.3042319 | 0.20496096 | 0.03643071 |
| FGFR2 | | | | | | | | | |
| PF4)- SDC2 | 0.04703636 | 0.00752054 | 0.09941627 | 0.0300415 | 0.01093616 | −0.1563344 | −0.1538259 | −0.2168714 | −0.1216114 |
| PF4)- LDLR | 0.02341923 | −0.0078504 | 0.02407892 | −0.1341327 | 0.25304697 | 0.1789973 | 0.07767054 | 0.03840706 | −0.0341908 |
| PF4)- | −0.154303 | 0.0732947 | −0.0699058 | 0.13048061 | 0.11800261 | 0.03333553 | 0.2059614 | 0.30850819 | 0.18466302 |
| THBD | | | | | | | | | |
| UCN)- | −0.2423546 | −0.2916437 | 0.15740099 | 0.03267457 | −0.087923 | 0.05454545 | −0.1141746 | −0.0793149 | 0.01870883 |
| CRHR1 | | | | | | | | | |
| UCN)- | −0.0829403 | −0.0589886 | −0.1282467 | 0.22121212 | 0.22701813 | −0.0353096 | 0.05515651 | 0.20131752 | 0.31185771 |
| CRHR2 | | | | | | | | | |
| TDGF1)- | 0.25408154 | −0.0366743 | 0.03136546 | 0.08669587 | 0.01660188 | −0.2394018 | 0.00415033 | −0.2897233 | 0.03610013 |
| SMAD3 | | | | | | | | | |
| TDGF1)- | 0.0030342 | 0.06048618 | −0.2359715 | 0.03794591 | 0.13495437 | −0.0299746 | −0.0164734 | 0.08616601 | −0.0888011 |
| ACVR1B | | | | | | | | | |
| LIPH)- | 0.42885375 | 0.26192358 | −0.1316422 | 0.20092227 | −0.2078076 | 0.25256917 | 0.18462147 | −0.320498 | −0.1837346 |
| LPAR1 | | | | | | | | | |
| LIPH)- | 0.28194993 | 0.1172014 | 0.09291905 | 0.12899401 | −0.1385219 | 0.25494071 | −0.0268881 | −0.4286044 | −0.1683302 |
| LPAR2 | | | | | | | | | |
| MELTF)- | 0.15068358 | 0.16814364 | 0.12380984 | 0.13083435 | 0.02292717 | 0.11620936 | 0.05217735 | −0.0264822 | −0.1470356 |
| TFRC | | | | | | | | | |
| SPINK1)- | 0.24361001 | 0.13017128 | −0.2641497 | 0.2740448 | −0.3245701 | 0.25335968 | 0.33023655 | −0.1990777 | −0.1931489 |
| NRSN1 | | | | | | | | | |
| IL3)- | 0.04522382 | −0.0187218 | 0.10144737 | 0.10089295 | −0.2350615 | 0.00481086 | 0.07959937 | −0.1117296 | 0.06469038 |
| CSF2RB | | | | | | | | | |
| IL3)- | −0.0541253 | −0.2901217 | 0.13700348 | −0.0280734 | 0.04574065 | −0.1093354 | 0.2354724 | −0.0601489 | 0.10131752 |
| IL3RA | | | | | | | | | |
| CSF2)- | −0.2129133 | 0.26459252 | 0.31231673 | −0.2137892 | −0.2546365 | 0.19488092 | −0.2479578 | 0.03003953 | −0.2772069 |
| ITGB1 | | | | | | | | | |
| CSF2)- | 0.11216218 | 0.44065794 | −0.1147595 | −0.1793985 | −0.0687008 | −0.253459 | −0.0911307 | −0.1297803 | 0.11014493 |
| CSF2RB | | | | | | | | | |
| CSF2)- | 0.2150297 | −0.0108105 | −0.1705989 | −0.1025134 | 0.12060955 | −0.1366451 | −0.1491229 | 0.03728713 | 0.1201581 |
| CSF2RA | | | | | | | | | |
| CSF2)- | 0.10738655 | −0.189974 | 0.16778194 | 0.18249498 | −0.0905677 | −0.2995553 | −0.0919752 | 0.23143817 | 0.18814229 |
| IL3RA | | | | | | | | | |
| CSF2)- | −0.1333509 | 0.16723249 | 0.21864808 | 0.12069704 | 0.10257931 | 0.09184043 | 0.16245059 | 0.12516469 | 0.06864295 |
| SDC2 | | | | | | | | | |
| CSF2)- | −0.0799578 | −0.0625556 | 0.07743553 | −0.0048094 | 0.14836181 | −0.0609415 | −0.0521407 | −0.1495389 | −0.2760211 |
| CSF3R | | | | | | | | | |
| SHH)- | 0.02678571 | 0.02431311 | −0.1148524 | 0.05362319 | 0.03425786 | 0.256341 | 0.01376902 | −0.0556031 | −0.0474308 |
| SCUBE2 | | | | | | | | | |
| SHH)- BOC | 0.10174629 | 0.08697371 | 0.11163097 | 0.13965744 | 0.21017262 | −0.3061668 | −0.1417842 | 0.02905521 | −0.0889328 |
| SHH)- | −0.0119263 | 0.07741978 | 0.03821824 | −0.2264822 | −0.214441 | 0.39632399 | 0.23288754 | −0.1629171 | 0.18814229 |
| GAS1 | | | | | | | | | |
| SHH)- | −0.0439495 | 0.10615096 | −0.014703 | 0.24230047 | 0.2096243 | −0.1237854 | 0.12946608 | 0.00955236 | 0.12984617 |
| PTCH1 | | | | | | | | | |
| SHH)- | −0.0660275 | 0.08433814 | 0.03192646 | 0.05059289 | 0.01528478 | 0.0110024 | 0.16773726 | −0.389103 | −0.0693017 |
| PTCH2 | | | | | | | | | |
| COL1A2)- | 0.25046113 | 0.14967062 | −0.0335968 | −0.2604743 | 0.46811594 | 0.38208169 | 0.11383399 | 0.34387352 | 0.01040843 |
| CD44 | | | | | | | | | |
| COL1A2)- | −0.2695652 | 0.11897233 | 0.27444005 | −0.1101449 | −0.1371542 | 0.82819499 | 0.28063241 | −0.1847167 | 0.13939394 |
| ITGB1 | | | | | | | | | |
| COL1A2)- | −0.4974472 | 0.58656126 | 0.58783227 | −0.472859 | −0.3674034 | 0.60944036 | 0.59349781 | −0.3934253 | 0.60500659 |
| ITGA11 | | | | | | | | | |
| COL1A2)- | 0.06318564 | 0.03333553 | −0.1058041 | 0.29073062 | 0.0378154 | 0.0598254 | −0.0663417 | 0.10732993 | 0.08926807 |
| ITGB3 | | | | | | | | | |
| COL1A2)- | 0.09842221 | 0.01054053 | −0.2223908 | −0.1009915 | −0.1040417 | 0.27398794 | −0.1366124 | 0.06871109 | −0.0193023 |
| CD36 | | | | | | | | | |
| DEFB1)- | −0.2624366 | −0.0250371 | 0.17285131 | 0.07483531 | 0.05594913 | 0.13880563 | −0.1003032 | 0.00718097 | 0.03965875 |
| CCR6 | | | | | | | | | |
| COL14A1)- | 0.09341238 | 0.03306983 | −0.0960474 | 0.28511199 | −0.3318841 | −0.5210804 | 0.09486166 | 0.24729908 | −0.18722 |
| CD44 | | | | | | | | | |
| ARF6)- | −0.056917 | 0.02483695 | −0.1442362 | 0.02483695 | −0.3907357 | −0.0317523 | 0.01495734 | 0.0088274 | 0.10600171 |
| PLD1 | | | | | | | | | |
| ARF6)- | 0.17994334 | 0.03491436 | 0.00626298 | 0.03965744 | −0.0742328 | 0.18817949 | 0.20186577 | −0.0900705 | −0.1075099 |
| SMAP1 | | | | | | | | | |
| JAM3)- | 0.02952224 | −0.0812521 | −0.0056672 | −0.1865261 | −0.2094548 | 0.00777467 | 0.09486166 | −0.1583663 | 0.06086957 |
| ITGB1 | | | | | | | | | |
| JAM3)- | 0.41713347 | 0.34359145 | −0.3958079 | −0.2988635 | 0.27296692 | −0.3043321 | −0.391131 | 0.34044796 | 0.30619236 |
| ITGB2 | | | | | | | | | |
| HSP90B1)- | −0.0967094 | 0.02292566 | −0.1566095 | 0.06811594 | −0.3175587 | 0.01620553 | 0.22291066 | −0.2077734 | −0.3475626 |
| TLR9 | | | | | | | | | |
| HSP90B1)- | 0.00836655 | −0.0379459 | 0.20897049 | 0.16073781 | −0.0781018 | 0.21620553 | 0.14150603 | −0.2032938 | −0.1945982 |
| TLR7 | | | | | | | | | |
| HSP90B1)- | 0.12174314 | 0.10889687 | −0.1549764 | 0.15665349 | −0.0021743 | 0.10250329 | −0.0975819 | −0.0638999 | −0.1324111 |
| TLR4 | | | | | | | | | |
| HSP90B1)- | 0.11963897 | −0.0962482 | 0.08821496 | −0.1445323 | 0.17932495 | 0.14783096 | −0.0014504 | −0.1735894 | −0.0131752 |
| TLR1 | | | | | | | | | |
| COL6A2)- | −0.3899868 | 0.22200264 | 0.41014493 | −0.1656126 | −0.2011858 | 0.69249012 | 0.46310935 | −0.51278 | 0.29064559 |
| ITGB1 | | | | | | | | | |
| ANGPTL4)- | −0.3277425 | −0.3870437 | 0.35009586 | −0.0962482 | −0.0211103 | −0.0481257 | −0.1467117 | −0.0225304 | −0.0899868 |
| TIE1 | | | | | | | | | |
| FADD)- | −0.1136102 | 0.27080877 | 0.37516091 | 0.25982411 | 0.05577901 | 0.1145003 | −0.0403877 | 0.32561357 | 0.12906839 |
| ABCA1 | | | | | | | | | |
| FADD)- | −0.0542858 | −0.0526299 | −0.1322803 | −0.1948617 | 0.04796258 | −0.0668072 | 0.19177044 | −0.0247751 | 0.13927595 |
| FAS | | | | | | | | | |
| FADD)- | 0.02810028 | 0.07465792 | 0.16280685 | −0.2466403 | 0.23804662 | 0.10060946 | −0.2569536 | −0.2309897 | −0.1071911 |
| TRADD | | | | | | | | | |
| CCL11)- | −0.5264512 | −0.0607879 | 0.12353252 | 0.02872673 | −0.1221329 | 0.23709608 | 0.16081729 | 0.08274045 | 0.19580973 |
| CCR3 | | | | | | | | | |
| CCL11)- | 0.18142268 | 0.21174071 | 0.00158327 | −0.2061333 | −0.1157663 | −0.1098887 | 0.10125917 | 0.25257749 | 0.01212121 |
| DPP4 | | | | | | | | | |
| NRTN)- | 0.06378492 | 0.01508912 | 0.18550143 | −0.2093613 | −0.1986816 | 0.18762764 | −0.3207647 | −0.1038277 | 0.35442538 |
| RET | | | | | | | | | |
| NRTN)- | −0.002537 | 0.10035252 | −0.003098 | −0.1572516 | −0.0398708 | 0.40837369 | −0.0340714 | 0.23691406 | 0.11541882 |
| GFRA1 | | | | | | | | | |
| IGF1)- | 0.08670123 | −0.0081789 | −0.0189638 | −0.090292 | 0.1979898 | −0.0488215 | −0.0063902 | 0.11558866 | 0.09380764 |
| INSR | | | | | | | | | |
| IGF1)- | −0.0347296 | 0.14161346 | 0.16997074 | −0.1183682 | 0.02435648 | −0.3364751 | −0.0453303 | −0.1729363 | 0.1229249 |
| IGF2R | | | | | | | | | |
| HSPA4)- | −0.2663108 | −0.0846184 | 0.1319623 | 0.15204216 | −0.0457271 | 0.18168643 | 0.11168215 | −0.2017128 | 0.09841897 |
| TLR4 | | | | | | | | | |
| PCSK9)- | 0.08764705 | −0.1242216 | 0.26808134 | 0.01956715 | 0.07280034 | 0.0193695 | −0.1038208 | −0.1131752 | 0.06930171 |
| LDLR | | | | | | | | | |
| SEMA3E)- | 0.00217477 | −0.0490971 | −0.092368 | 0.22681907 | −0.0055341 | 0.15843737 | −0.0527703 | 0.13399209 | 0.39657444 |
| PLXND1 | | | | | | | | | |
| IL12A)- | 0.01575634 | −0.0770347 | 0.06510847 | 0.3765729 | −0.1710427 | 0.35753343 | 0.35947949 | −0.1216154 | 0.10251005 |
| CD28 | | | | | | | | | |
| CD14)- | −0.1398781 | −0.0579806 | 0.018358 | 0.07641634 | −0.0413234 | 0.14571805 | −0.0605022 | 0.16389987 | 0.07944664 |
| ITGA4 | | | | | | | | | |
| CD14)- | −0.0213474 | −0.0917147 | −0.0767584 | 0.17839262 | 0.1972332 | −0.128722 | 0.2516469 | −0.1450593 | 0.22714097 |
| ITGB1 | | | | | | | | | |
| CD14)- | −0.1568162 | −0.1711142 | 0.01226104 | 0.17253533 | 0.01753172 | 0.22365691 | 0.048377 | 0.16212655 | 0.1993478 |
| TLR6 | | | | | | | | | |
| CD14)- | −0.0034261 | 0.14791633 | 0.06813013 | −0.1252964 | −0.0116003 | 0.09051383 | 0.03110994 | 0.02621871 | −0.1362319 |
| TLR9 | | | | | | | | | |
| CD14)- | −0.3341789 | −0.2642069 | 0.29688942 | 0.41119895 | −0.3571311 | 0.51620553 | −0.4020031 | 0.56916996 | 0.49459816 |
| ITGB2 | | | | | | | | | |
| CD14)- | −0.1709768 | −0.3252841 | 0.24521401 | 0.28471673 | −0.2335112 | 0.16798419 | −0.2825328 | 0.1828722 | 0.34018445 |
| TLR4 | | | | | | | | | |
| CD14)- | −0.2406273 | −0.3972986 | 0.23560059 | 0.37338603 | −0.2612079 | 0.39836622 | −0.2829642 | 0.33933924 | 0.37312253 |
| TLR1 | | | | | | | | | |
| COL3A1)- | −0.2123258 | 0.34176548 | 0.39710098 | −0.1321476 | −0.1158294 | 0.29921934 | 0.40994894 | −0.1945387 | 0.34295125 |
| DDR2 | | | | | | | | | |
| COL3A1)- | −0.1758893 | 0.11515152 | 0.24176548 | −0.144137 | −0.1768116 | 0.80250329 | 0.28893281 | −0.1476943 | 0.17496706 |
| ITGB1 | | | | | | | | | |
| COL3A1)- | 0.04387496 | −0.0789196 | 0.06154049 | 0.00658762 | −0.0489557 | 0.0352449 | 0.00177901 | 0.05019928 | −0.0279315 |
| MAG | | | | | | | | | |
| COL3A1)- | 0.27483531 | −0.1546772 | 0.11370224 | 0.05428195 | −0.1806324 | −0.2575758 | 0.18366271 | 0.36679842 | −0.2743083 |
| DDR1 | | | | | | | | | |
| IL13)- | −0.1926361 | 0.06190054 | 0.04334927 | 0.12959547 | 0.00016473 | 0.01937142 | −0.0010542 | −0.0995487 | 0.27945585 |
| IL13RA2 | | | | | | | | | |
| IL13)- | −0.0234681 | 0.06763796 | 0.04126703 | 0.08624609 | 0.02180788 | 0.18876005 | 0.1294509 | 0.27431734 | −0.2362474 |
| TMEM219 | | | | | | | | | |
| IL13)- | 0.07004418 | −0.066581 | 0.10409866 | −0.1720912 | 0.02016873 | −0.1533849 | 0.09964742 | 0.05481257 | 0.01152871 |
| IL2RG | | | | | | | | | |
| IL13)- | −0.0536603 | −0.1764066 | 0.23651592 | −0.2130057 | 0.00128505 | −0.1963368 | −0.0899447 | 0.09868573 | −0.0992786 |
| IL13RA1 | | | | | | | | | |
| IL13)- IL4R | −0.0637463 | 0.00758125 | 0.06641792 | 0.07007083 | −0.0923256 | 0.32632758 | 0.03109122 | 0.21193056 | −0.0455234 |
| NLGN2)- | −0.0119918 | −0.0042168 | 0.22573098 | 0.22250774 | −0.2990507 | 0.28636379 | 0.09900796 | 0.1009948 | 0.01350506 |
| NRXN1 | | | | | | | | | |
| CXCL10)- | 0.03249407 | 0.1556105 | −0.0046499 | −0.1179222 | −0.1671227 | 0.10936162 | −0.279056 | 0.19361639 | −0.0296443 |
| DPP4 | | | | | | | | | |
| CXCL10)- | 0.09965399 | −0.0610974 | 0.22178286 | −0.0657466 | −0.0648901 | −0.0637702 | −0.0818182 | −0.0843215 | −0.0014493 |
| SDC4 | | | | | | | | | |
| CXCL10)- | 0.1344538 | −0.0108105 | −0.2599407 | 0.00250371 | −0.1302399 | 0.14506407 | 0.24485091 | 0.15059289 | 0.17050995 |
| CCR3 | | | | | | | | | |
| BMP1)- | −0.1672267 | −0.0556745 | 0.10963959 | 0.24677207 | 0.10020093 | 0.01923647 | −0.0252314 | −0.0337956 | 0.07101449 |
| BMPR2 | | | | | | | | | |
| BMP1)- | −0.2591421 | 0.13230111 | 0.04667722 | 0.00777339 | 0.17758153 | 0.05487664 | −0.1467981 | −0.0034916 | 0.13306983 |
| BMPR1A | | | | | | | | | |
| BMP1)- | −0.0112682 | −0.1425136 | 0.21091847 | 0.19934124 | 0.05319887 | 0.18171037 | −0.129734 | 0.03070233 | 0.04611331 |
| BMPR1B | | | | | | | | | |
| FGB)- | 0.14136489 | 0.25768612 | −0.2073979 | 0.17752299 | −0.0967136 | 0.01324503 | −0.0375033 | 0.13492325 | 0.07398379 |
| ITGAM | | | | | | | | | |
| FGB)- | −0.172999 | 0.23877156 | 0.27620525 | 0.10220422 | 0.02714902 | −0.1157787 | 0.04203175 | −0.1199684 | 0.02318993 |
| ITGB1 | | | | | | | | | |
| FGB)- | −0.0255092 | −0.0992585 | −0.1404844 | 0.2007974 | −0.0782233 | 0.17959534 | 0.10870997 | −0.2499918 | −0.1232046 |
| ITGB3 | | | | | | | | | |
| FGB)- | 0.26856034 | 0.24153955 | −0.2238299 | 0.09686666 | 0.04798313 | 0.04322757 | 0.14167573 | −0.0801107 | −0.032545 |
| ITGB2 | | | | | | | | | |
| FGB)- | −0.0575345 | −0.1264705 | −0.085396 | 0.22813088 | −0.1639809 | 0.34918126 | 0.23906168 | −0.1270835 | −0.0393307 |
| TLR4 | | | | | | | | | |
| FGA)- | 0.04333204 | 0.05052108 | −0.0538766 | −0.0770776 | 0.12476521 | −0.1577127 | −0.198906 | 0.20606061 | 0.43241107 |
| ITGAM | | | | | | | | | |
| FGA)- | 0.05909515 | 0.27074284 | −0.1407225 | 0.09881748 | −0.0305766 | 0.03241213 | 0.00685315 | 0.34374177 | 0.28656126 |
| ITGAX | | | | | | | | | |
| FGA)- | 0.07960697 | −0.1435168 | −0.1360639 | 0.25626668 | 0.23979709 | −0.1455252 | 0.0629776 | −0.159552 | 0.01620553 |
| ITGB1 | | | | | | | | | |
| FGA)- | −0.0939191 | 0.01833532 | 0.06587066 | 0.17266708 | 0.15709015 | −0.0629797 | 0.24254605 | 0.24967062 | −0.0283267 |
| PLAUR | | | | | | | | | |
| FGA)- | 0.10191634 | 0.10348928 | 0.15652012 | 0.08933689 | −0.210561 | 0.05363379 | −0.0568549 | 0.01607643 | 0.14065485 |
| ITGB3 | | | | | | | | | |
| FGA)- | −0.1169372 | 0.06216408 | 0.23052323 | −0.0231899 | −0.1195269 | 0.09308607 | −0.004744 | 0.128722 | 0.42590336 |
| CDH5 | | | | | | | | | |
| FGA)- | 0.06898831 | 0.05190612 | −0.0720388 | 0.1397938 | −0.0762717 | 0.12418064 | −0.1105657 | 0.23623188 | 0.16837945 |
| ITGB2 | | | | | | | | | |
| FGA)- | −0.258871 | −0.0495977 | 0.20799535 | 0.32530716 | −0.3854644 | 0.31555717 | −0.0838769 | 0.16666667 | 0.20711462 |
| TLR4 | | | | | | | | | |
| CXCL8)- | 0.1735951 | 0.03649779 | 0.23130641 | −0.0732543 | 0.06956522 | 0.10131752 | 0.31791831 | 0.05955204 | 0.41936759 |
| SDC1 | | | | | | | | | |
| CXCL8)- | −0.280058 | 0.05145266 | 0.29565539 | −0.0160738 | 0.05946339 | −0.0180501 | 0.47366352 | −0.2575758 | 0.40803689 |
| SDC3 | | | | | | | | | |
| CXCL8)- | −0.0929574 | 0.01956651 | 0.08379999 | 0.21660079 | 0.33017128 | −0.1243742 | −0.1084321 | 0.05006588 | −0.0801054 |
| SDC2 | | | | | | | | | |
| CXCL8)- | −0.2493741 | −0.1014034 | 0.17145977 | 0.14389248 | −0.2105265 | 0.07899071 | −0.0435959 | −0.0062586 | 0.01660298 |
| CXCR2 | | | | | | | | | |
| SEMA4C)- | −0.2420269 | 0.18674223 | −0.2026226 | −0.2748353 | 0.10737813 | 0.09038208 | −0.1667984 | −0.1480896 | 0.06337286 |
| PLXNB2 | | | | | | | | | |
| CXCL11)- | −0.1800237 | 0.19817447 | 0.0423413 | 0.05171789 | −0.2263393 | 0.07764528 | 0.09110689 | −0.0231233 | 0.05862978 |
| DPP4 | | | | | | | | | |
| CXCL11)- | 0.05496425 | 0.20525328 | −0.081564 | 0.01423253 | 0.14386001 | 0.01508713 | 0.08983359 | 0.01528327 | −0.0944789 |
| CCR3 | | | | | | | | | |
| PRND)- | −0.0487532 | −0.2253772 | 0.05912969 | −0.1555951 | 0.25775202 | 0.17846905 | 0.16700155 | 0.18742383 | −0.4061924 |
| RPSA | | | | | | | | | |
| HAS2)- | 0.11892604 | −0.0662823 | 0.19428063 | 0.10764163 | 0.06581461 | −0.1424242 | −0.1150273 | 0.39222661 | 0.00197628 |
| HMMR | | | | | | | | | |
| HAS2)- | 0.34037227 | −0.1560863 | 0.19838577 | −0.140975 | 0.20750988 | 0.20131752 | 0.17325428 | 0.16890646 | −0.215942 |
| CD44 | | | | | | | | | |
| NPTX1)- | −0.12479 | 0.03109764 | 0.05036478 | −0.1383399 | −0.377191 | 0.36239665 | 0.15351643 | −0.0054679 | 0.33320158 |
| NPTXR | | | | | | | | | |
| RGMB)- | 0.30293857 | −0.0891704 | −0.1599447 | 0.13983065 | 0.31341023 | −0.1294234 | 0.16509108 | 0.06317731 | −0.0305665 |
| BMPR2 | | | | | | | | | |
| RGMB)- | −0.2467187 | 0.32654026 | −0.0629659 | −0.1306712 | 0.26060307 | −0.1191473 | 0.00507347 | −0.1557415 | 0.02160738 |
| NEO1 | | | | | | | | | |
| RGMB)- | −0.3337733 | −0.1098141 | 0.04577105 | 0.27096308 | −0.2204748 | 0.22077965 | 0.02808267 | −0.0404533 | 0.03583663 |
| BMPR1B | | | | | | | | | |
| F2)- GP1BB | 0.15215314 | 0.11919745 | −0.0181543 | −0.0809671 | −0.0695744 | −0.0420345 | 0.07787331 | 0.02292717 | −0.0830726 |
| F2)- F2R | −0.143709 | 0.09422463 | 0.261984 | 0.18374069 | 0.02272877 | −0.000527 | 0.14302184 | 0.07048981 | 0.3589051 |
| F2)- THBD | 0.09699206 | −0.0546587 | −0.238223 | 0.16068782 | 0.08842769 | 0.21384808 | 0.12225658 | 0.01890708 | 0.01963239 |
| F2)- GP1BA | −0.1400283 | −0.2607321 | 0.13288475 | 0.13169511 | 0.14582589 | 0.01264989 | 0.07599452 | −0.1434266 | 0.24427682 |
| CCL19)- | −0.2654675 | −0.1661066 | 0.47358015 | 0.16575532 | −0.2463993 | 0.30515844 | −0.044755 | −0.1708827 | −0.0426877 |
| CCRL2 | | | | | | | | | |
| CLCF1)- | −0.1344051 | −0.1368518 | 0.11106351 | 0.22375226 | 0.03219747 | −0.0232895 | 0.17248313 | 0.05474669 | 0.04177098 |
| CRLF1 | | | | | | | | | |
| CLCF1)- | −0.0002635 | 0.15686662 | 0.00856531 | −0.0631794 | −0.0914811 | 0.22181962 | 0.19926211 | −0.2874271 | −0.012451 |
| IL6ST | | | | | | | | | |
| CSHL1)- | 0.21270918 | 0.21068582 | −0.1455498 | 0.11986821 | 0.06575695 | 0.11608673 | −0.0044153 | 0.04137295 | −0.0437418 |
| GHR | | | | | | | | | |
| LPL)- CD44 | 0.02118676 | 0.07603472 | 0.13147671 | −0.0515168 | 0.03326855 | −0.0591587 | 0.04875478 | −0.1448149 | −0.1325603 |
| LPL)- SDC1 | 0.14018902 | −0.1325988 | 0.02085675 | 0.14783096 | −0.1127178 | −0.2639086 | −0.2054289 | −0.2601133 | 0.33008302 |
| EFEMP2)- | −0.0441385 | 0.00395257 | 0.13343877 | 0.05270787 | −0.2154414 | 0.19937407 | 0.09950249 | −0.1413749 | 0.02490119 |
| AQP1 | | | | | | | | | |
| EFEMP2)- | −0.1926346 | −0.0997365 | 0.14652304 | 0.22624852 | −0.1778407 | 0.23285234 | 0.03360016 | −0.1562685 | −0.0555995 |
| PLSCR4 | | | | | | | | | |
| ADAM17)- | −0.041267 | 0.10771617 | −0.0013844 | −0.1923584 | 0.08194993 | 0.07549407 | 0.21858321 | 0.27024713 | −0.4579242 |
| MUC1 | | | | | | | | | |
| ADAM17)- | 0.0142391 | −0.088467 | −0.0415966 | −0.0557312 | −0.0301713 | 0.33596838 | 0.09746294 | 0.16836904 | 0.15677101 |
| ITGB1 | | | | | | | | | |
| ADAM17)- | −0.2054781 | −0.0872174 | 0.20575047 | 0.36371422 | 0.02537151 | −0.0167325 | 0.3109529 | −0.0119934 | 0.05179743 |
| RHBDF2 | | | | | | | | | |
| ADAM17)- | −0.2641908 | 0.1641452 | 0.01984637 | −0.0886693 | 0.04888977 | −0.0475939 | 0.02606776 | 0.09297726 | 0.06425042 |
| IL6R | | | | | | | | | |
| ADAM17)- | −0.3574937 | 0.11529717 | 0.03065362 | 0.25507246 | −0.2536232 | −0.0422925 | 0.02800659 | 0.10754531 | −0.1424712 |
| MET | | | | | | | | | |
| VEGFB)- | −0.2714935 | −0.1895319 | 0.07422546 | 0.17352922 | −0.3662613 | 0.45241625 | 0.11443264 | −0.3130538 | −0.1032938 |
| RET | | | | | | | | | |
| LEP)- LEPR | −0.07278 | −0.0498995 | 0.02722658 | 0.17431404 | 0.06311767 | 0.11213599 | −0.2041704 | 0.09322397 | 0.3314888 |
| GH1)- GHR | 0.19626203 | 0.20587368 | 0.03851227 | −0.129249 | −0.3907213 | 0.03735424 | −0.3090712 | 0.25765861 | 0.07351779 |
| GNAI2)- | −0.2183639 | −0.005337 | 0.31823875 | −0.1627141 | −0.198102 | 0.18584275 | −0.0785554 | 0.09222965 | −0.2047431 |
| S1PR5 | | | | | | | | | |
| GNAI2)- | 0.44702162 | −0.1927258 | −0.1678197 | 0.25586298 | 0.0972332 | −0.2645102 | 0.18392622 | −0.1690493 | 0.30671937 |
| EDNRA | | | | | | | | | |
| GNAI2)- | −0.1716413 | −0.2399684 | 0.4030387 | −0.0296443 | −0.0735469 | 0.05915679 | 0.00566759 | −0.0017128 | −0.0375494 |
| C5AR1 | | | | | | | | | |
| GNAI2)- | 0.10358357 | 0.03907228 | −0.1223831 | 0.00408432 | 0.07588096 | −0.3418703 | 0.26133112 | −0.5180792 | 0.08445323 |
| ADRA2B | | | | | | | | | |
| GNAI2)- | −0.1062134 | 0.17987745 | 0.1960203 | −0.0072464 | 0 | −0.1463768 | 0.10144928 | −0.3665349 | 0.05757576 |
| F2R | | | | | | | | | |
| GNAI2)- | −0.1815247 | −0.349608 | 0.34398999 | 0.32068511 | −0.2547863 | 0.16007905 | −0.0554256 | −0.0055336 | 0.07220026 |
| FPR1 | | | | | | | | | |
| GNAI2)- | 0.00296501 | −0.1423865 | −0.1292694 | 0.17009223 | −0.0682972 | 0.11805007 | 0.0746259 | 0.08906456 | 0.08919631 |
| S1PR3 | | | | | | | | | |
| GNAI2)- | 0.048267 | −0.2565066 | −0.2675101 | −0.0162055 | −0.0600791 | 0.2141116 | −0.1151515 | 0.17530799 | −0.0364954 |
| UNC5B | | | | | | | | | |
| GNAI2)- | 0.50266852 | −0.1953614 | −0.0894804 | 0.028722 | 0.01133107 | 0.0743083 | −0.0180507 | 0.03741765 | 0.13952569 |
| CAV1 | | | | | | | | | |
| GNAI2)- | −0.2164531 | 0.21558938 | −0.0293284 | −0.116996 | 0.04520295 | 0.04980401 | 0.18819189 | −0.0347179 | −0.0180501 |
| PTPRU | | | | | | | | | |
| GNAI2)- | −0.0994992 | 0.01363906 | 0.0672532 | 0.25665349 | 0.02523555 | −0.0121879 | 0.05225012 | −0.1492193 | 0.12068511 |
| TBXA2R | | | | | | | | | |
| GNAI2)- | −0.1569584 | 0.11247282 | 0.04019902 | −0.0275362 | 0.0060616 | −0.0230582 | 0.0720145 | −0.216088 | −0.0711462 |
| EDNRB | | | | | | | | | |
| GNAI2)- | −0.0608197 | 0.13449753 | 0.26558493 | −0.0134405 | 0.01134416 | 0.06271823 | 0.05896324 | 0.03623427 | −0.2261167 |
| CXCR2 | | | | | | | | | |
| GNAI2)- | −0.0708332 | −0.0952099 | −0.1796283 | 0.19986825 | 0.06734318 | 0.07503541 | 0.24920929 | −0.1706249 | 0.09393939 |
| DRD2 | | | | | | | | | |
| GNAI2)- | 0.26712352 | −0.0977136 | 0.03273987 | 0.11330698 | −0.0659283 | 0.03300504 | −0.1791272 | 0.17602688 | 0.20184453 |
| ADCY7 | | | | | | | | | |
| GNAI2)- | −0.1740577 | 0.0225341 | 0.08566343 | −0.0515152 | 0.00698886 | −0.0664075 | 0.07845984 | −0.2763687 | 0.02226614 |
| AGTR2 | | | | | | | | | |
| ANGPT2)- | 0.10858462 | 0.36934031 | 0.24617223 | −0.0541591 | −0.2233367 | 0.03801805 | −0.0678828 | −0.0855788 | 0.0920979 |
| TIE1 | | | | | | | | | |
| TLN1)- | −0.1588588 | 0.1102217 | 0.13502652 | −0.0119906 | −0.2647824 | 0.0834157 | −0.1415772 | −0.0370944 | 0.0692404 |
| ITGB3 | | | | | | | | | |
| AGTRAP)- | −0.1188067 | 0.25383229 | −0.1658151 | −0.3764659 | 0.30689156 | 0.25919094 | 0.43504611 | 0.51884058 | −0.4965744 |
| RACK1 | | | | | | | | | |
| PKM)- | 0.14137022 | −0.3134387 | 0.04440053 | −0.4508564 | 0.0831357 | 0.12687747 | 0.13715415 | 0.14611331 | 0.28722003 |
| CD44 | | | | | | | | | |
| MMP1)- | 0.372916 | 0.16151566 | 0.0893575 | 0.1027668 | −0.073913 | 0.06455863 | 0.05586298 | −0.0519104 | −0.2223979 |
| CD44 | | | | | | | | | |
| LAMA3)- | −0.2129117 | 0.37378129 | 0.37246377 | 0.32476943 | 0.31554677 | −0.2086957 | −0.1646904 | 0.21238472 | −0.2144928 |
| ITGB1 | | | | | | | | | |
| MTMR4)- | 0.01034938 | 0.24601192 | 0.01163519 | 0.12187891 | −0.3522087 | −0.2130575 | 0.00563278 | −0.0913074 | 0.04828881 |
| SMAD3 | | | | | | | | | |
| WNT11)- | −0.2684856 | −0.0633812 | −0.1270798 | 0.00810303 | −0.2447453 | 0.14160984 | 0.07412288 | 0.03927254 | 0.17140975 |
| KLRG2 | | | | | | | | | |
| PSEN1)- | 0.1025641 | 0.10332104 | 0.03064755 | −0.0462496 | −0.1260297 | 0.15447985 | −0.0753492 | 0.0058651 | 0.0975658 |
| NOTCH4 | | | | | | | | | |
| PSEN1)- | 0.19807592 | −0.10767 | 0.17369531 | 0.01120005 | −0.1912574 | −0.428962 | 0.11910801 | 0.1957245 | 0.04275503 |
| CD44 | | | | | | | | | |
| BGN)- | 0.19802372 | 0.22002635 | −0.2607235 | −0.0889328 | 0.42274495 | −0.2905138 | −0.3728668 | 0.35191041 | 0.27971014 |
| TLR4 | | | | | | | | | |
| BGN)- | 0.16970256 | −0.0542819 | −0.1317247 | −0.1501976 | 0.01832806 | −0.0592905 | −0.1086498 | 0.23670081 | 0.01027668 |
| TLR1 | | | | | | | | | |
| CSF1)- | −0.1276105 | 0.27496706 | −0.0047453 | −0.02813 | −0.0788268 | 0.03821194 | 0.08877612 | −0.2936294 | −0.0833992 |
| SIRPA | | | | | | | | | |
| P4HB)- | 0.07869684 | 0.03939524 | −0.123937 | −0.0986825 | −0.1140447 | 0.13874432 | 0.1462804 | −0.1382173 | −0.3370224 |
| MTTP | | | | | | | | | |
| JAG2)- | 0.01028108 | −0.0034259 | 0.09897858 | 0.00863034 | −0.2435505 | 0.20469224 | −0.0111356 | −0.2022405 | −0.1263505 |
| NOTCH4 | | | | | | | | | |
| CTHRC1)- | 0.09447885 | −0.1868309 | 0.09203808 | −0.1298505 | −0.1476479 | −0.2234887 | 0.20733274 | 0.21214917 | −0.3640436 |
| FZD5 | | | | | | | | | |
| CTHRC1)- | −0.0950247 | −0.2475626 | 0.03597312 | 0.28367206 | −0.0274749 | 0.01462981 | 0.09276585 | −0.041318 | −0.269697 |
| FZD6 | | | | | | | | | |
| CTHRC1)- | 0.1828963 | 0.02332016 | 0.06351926 | −0.0354425 | 0.20357802 | 0.12386757 | 0.12842224 | 0.15113981 | 0.06363636 |
| FZD3 | | | | | | | | | |
| FBLN1)- | −0.116996 | 0.0798419 | 0.1544137 | −0.1230567 | −0.1168643 | 0.4685112 | 0.15283267 | −0.1172596 | 0.07483531 |
| ITGB1 | | | | | | | | | |
| CALCA)- | 0.10197424 | 0.27519615 | 0.00270333 | −0.0440871 | 0.23757416 | 0.206208 | 0.12829891 | 0.19196943 | 0.13227931 |
| CALCRL | | | | | | | | | |
| CALCA)- | −0.1911542 | −0.1705887 | 0.43131699 | −0.0327523 | −0.0122932 | 0.04079212 | 0.05363556 | 0.17285903 | 0.12213439 |
| GPR84 | | | | | | | | | |
| CALCA)- | −0.3607239 | −0.2508734 | 0.10549052 | 0.25793273 | −0.0437162 | 0.0427404 | 0.06096959 | −0.0604144 | 0.39249012 |
| CALCR | | | | | | | | | |
| CALCA)- | 0.19745518 | 0.27827037 | −0.32232 | 0.04866878 | 0.30015824 | −0.2451636 | 0.21598289 | 0.04407986 | −0.0900557 |
| ADRB3 | | | | | | | | | |
| COL4A5)- | 0.03439868 | 0.00342669 | 0.22158886 | 0.04216074 | −0.06713 | 0.05401845 | −0.1033665 | −0.0333344 | 0.17826674 |
| CD47 | | | | | | | | | |
| COL4A5)- | −0.0249094 | −0.1556507 | −0.1855025 | 0.05256917 | 0.05032938 | −0.1006588 | 0.07325669 | 0.16983432 | −0.0012517 |
| ITGB1 | | | | | | | | | |
| SLIT3)- | −0.0733733 | 0.01325028 | −0.0155591 | 0.0695698 | 0.02665129 | 0.1721514 | −0.0021082 | −0.175961 | 0.25757576 |
| ROBO1 | | | | | | | | | |
| COL7A1)- | −0.1590552 | 0.06824193 | 0.15918718 | −0.1728045 | −0.1436854 | 0.47737005 | 0.43565335 | −0.1982938 | 0.31167035 |
| ITGB1 | | | | | | | | | |
| GHRL)- | 0.13927431 | −0.0207619 | 0.06383891 | 0.12569998 | −0.1488997 | 0.10073459 | −0.0553396 | 0.14289008 | 0.09183136 |
| PTGER3 | | | | | | | | | |
| GHRL)- | −0.2205854 | 0.31096762 | 0.03256642 | 0.08314118 | 0.1737612 | 0.25372249 | 0.13665415 | −0.1695764 | 0.13649539 |
| TBXA2R | | | | | | | | | |
| HSP90AA1)- | −0.0615385 | 0.46603861 | 0.12649055 | 0.46702681 | 0.03801304 | −0.0745841 | 0.11963898 | −0.2090595 | −0.4566177 |
| ITGB3 | | | | | | | | | |
| HSP90AA1)- | −0.1632411 | 0.42267532 | −0.2233717 | 0.41233242 | −0.1974105 | −0.2018445 | 0.25822819 | 0.0312253 | −0.2042228 |
| FGFR3 | | | | | | | | | |
| FGG)- | 0.0078403 | −0.1151667 | −0.0399262 | 0.10066539 | 0.10560643 | −0.1753739 | −0.0474324 | 0.06297968 | −0.0369577 |
| ITGB1 | | | | | | | | | |
| FGG)- | −0.0033607 | 0.11224221 | 0.1360282 | −0.3305442 | 0.06740018 | −0.1349455 | −0.2858319 | 0.25433222 | 0.13169944 |
| ITGB3 | | | | | | | | | |
| FGG)- | 0.27111609 | 0.2150481 | −0.0897888 | 0.10303709 | −0.2074726 | 0.03827657 | −0.1387388 | 0.09809282 | 0.09486478 |
| ITGB2 | | | | | | | | | |
| FGG)- | −0.0164712 | 0.02253261 | −0.0531796 | −0.0611371 | 0.08655113 | −0.1524475 | −0.2320693 | 0.39579696 | 0.20218057 |
| TLR4 | | | | | | | | | |
| CXCL14)- | 0.22345927 | 0.18755558 | −0.2033731 | −0.1150198 | 0.21390691 | −0.1687747 | 0.07062156 | −0.071278 | −0.1748353 |
| CXCR4 | | | | | | | | | |
| EDN3)- | 0.0853261 | 0.10039255 | −0.2049084 | −0.0592885 | 0.21946961 | 0.13801963 | −0.3552627 | 0.12201067 | 0.09828722 |
| EDNRB | | | | | | | | | |
| EDN3)- | −0.1166887 | 0.06477364 | 0.11914116 | −0.257444 | −0.1652392 | 0.15270595 | −0.1301884 | 0.01113344 | 0.29472991 |
| KEL | | | | | | | | | |
| RTN4)- | 0.21140354 | −0.0862347 | 0.03309164 | −0.1643059 | 0.07798545 | 0.16351538 | −0.091038 | 0.44746031 | −0.0411094 |
| TNFRSF19 | | | | | | | | | |
| RTN4)- | 0.17406773 | −0.0144928 | 0.1279357 | −0.0994104 | −0.0260887 | 0.36468457 | 0.13755847 | 0.37812552 | −0.102309 |
| GJB2 | | | | | | | | | |
| WNT7B)- | −0.0666096 | −0.0241138 | 0.07378348 | −0.027668 | 0.26931707 | −0.1523057 | −0.2227058 | 0.07628458 | 0.02371542 |
| FZD4 | | | | | | | | | |
| WNT7B)- | −0.0753722 | 0.06825669 | 0.11167496 | 0.12674572 | 0.09857995 | 0.1455863 | 0.01891206 | 0.14914361 | 0.02700922 |
| TMED5 | | | | | | | | | |
| PTN)- | −0.2094303 | 0.28745595 | −0.0117302 | −0.1921202 | −0.1905389 | −0.0961919 | −0.2183136 | −0.2397892 | 0.05546772 |
| SDC1 | | | | | | | | | |
| PTN)- | −0.1758213 | 0.14741838 | 0.05783626 | −0.2201871 | −0.1946991 | 0.10844644 | −0.2034413 | 0.1314888 | −0.0549407 |
| SDC3 | | | | | | | | | |
| PTN)- | −0.0218158 | 0.01061025 | −0.1085415 | −0.012255 | 0.05660008 | 0.21151819 | −0.1914123 | −0.0747134 | 0.2300471 |
| PTPRZ1 | | | | | | | | | |
| PTN)- | −0.1614934 | −0.1433326 | −0.0306237 | 0.10146264 | −0.0540487 | 0.23468951 | −0.3429664 | 0.14941204 | −0.031357 |
| PTPRS | | | | | | | | | |
| COL9A3)- | −0.0310379 | 0.12573312 | 0.06906096 | 0.24295925 | 0.23241872 | 0.12121611 | 0.09802372 | −0.027668 | 0.05046113 |
| ITGB1 | | | | | | | | | |
| COL9A3)- | −0.1271871 | −0.0358484 | 0.05866069 | 0.11014856 | 0.05086812 | 0.17708676 | 0.03538249 | −0.0009223 | 0.0201581 |
| MAG | | | | | | | | | |
| SLPI)- | −0.1100863 | −0.0711462 | −0.2345423 | −0.1330698 | −0.2359258 | −0.0606759 | 0.15363837 | 0.05277027 | 0.13109354 |
| PLSCR4 | | | | | | | | | |
| HMGB1)- | −0.0573236 | 0.16485472 | −0.2132833 | 0.29209486 | −0.027668 | 0.02819499 | 0.60698287 | 0.48115942 | −0.1361001 |
| SDC1 | | | | | | | | | |
| HMGB1)- | −0.078474 | −0.0531067 | −0.0714945 | −0.1909091 | −0.1107303 | 0.00447958 | −0.1883733 | −0.1254282 | −0.1314888 |
| TLR9 | | | | | | | | | |
| HMGB1)- | 0.07919879 | 0.03258327 | −0.2703689 | −0.0189071 | −0.2408085 | −0.0274045 | 0.13050394 | −0.1955204 | −0.4421094 |
| THBD | | | | | | | | | |
| HMGB1)- | −0.0855242 | 0.00981749 | 0.11579017 | −0.0017128 | −0.1280227 | 0.24677207 | 0.25011531 | −0.0463768 | 0.01014493 |
| TLR4 | | | | | | | | | |
| HMGB1)- | −0.0467813 | −0.0316268 | 0.28886766 | 0.05638999 | −0.0504628 | 0.01014493 | 0.12049145 | −0.3437418 | −0.3915679 |
| CXCR4 | | | | | | | | | |
| HMGB1)- | 0.18337287 | 0.46499523 | −0.0162529 | 0.24381567 | −0.171067 | −0.0206233 | −0.1077821 | −0.2735719 | −0.4894101 |
| AGER | | | | | | | | | |
| HMGB1)- | −0.3330698 | −0.3845292 | 0.43111148 | −0.2499341 | 0.23849901 | −0.2357049 | −0.0039574 | −0.0869565 | −0.0561265 |
| CD163 | | | | | | | | | |
| FYN)- SPN | −0.1561573 | −0.1666227 | 0.31598451 | 0.2226614 | −0.3129069 | 0.25100468 | −0.3049208 | 0.11904605 | 0.2483531 |
| FN1)- | 0.30632411 | 0.33662714 | −0.433731 | −0.1698287 | 0.3762605 | −0.1902503 | −0.3924735 | 0.32779974 | 0.35981555 |
| ITGA4 | | | | | | | | | |
| FN1)- | −0.0320211 | 0.16871994 | −0.0776731 | 0.3552935 | 0.11160156 | 0.11148081 | −0.1226036 | 0.00276725 | 0.29712102 |
| ITGB3 | | | | | | | | | |
| FN1)- | 0.12911726 | 0.17562582 | −0.2282193 | −0.1820817 | 0.33972587 | −0.1629776 | −0.1267958 | 0.12187088 | 0.15915679 |
| C5AR1 | | | | | | | | | |
| FN1)- | 0.50184453 | −0.1606061 | 0.45085639 | 0.08392622 | −0.1753623 | −0.2301713 | 0.49328063 | 0.53096179 | −0.2923584 |
| ITGA6 | | | | | | | | | |
| FN1)- | 0.01666776 | 0.38136961 | 0.07287 | −0.0256267 | 0.12424169 | −0.2378945 | 0.23173318 | 0.01805126 | 0.41081722 |
| ITGA8 | | | | | | | | | |
| FN1)- DPP4 | −0.1205573 | 0.05797101 | −0.2544005 | 0.09222661 | −0.0058672 | 0.18268059 | −0.2769465 | −0.0891992 | 0.07101449 |
| FN1)- CD44 | 0.04782609 | 0.27799736 | −0.2591568 | −0.3826087 | 0.52147563 | 0.36864295 | −0.1903821 | 0.02318841 | 0.22674572 |
| FN1)- | −0.3640316 | 0.28642951 | 0.42424242 | −0.0986825 | −0.1637681 | 0.78774704 | 0.41185771 | −0.3028986 | 0.29947299 |
| ITGB1 | | | | | | | | | |
| FN1)- | −0.1061994 | −0.1919631 | 0.13147057 | 0.20948617 | −0.1978978 | 0.23124053 | 0.11868596 | −0.0183148 | −0.116996 |
| COL13A1 | | | | | | | | | |
| FN1)- MAG | −0.027603 | −0.1121212 | 0.00171312 | −0.1773386 | 0.126639 | 0.01113344 | −0.0648349 | 0.00309628 | −0.0716733 |
| COL18A1)- | −0.0529177 | 0.32251477 | 0.3814294 | −0.1186469 | −0.1556705 | 0.40409763 | 0.22793149 | −0.0508564 | 0.1859025 |
| ITGB1 | | | | | | | | | |
| COL18A1)- | −0.018455 | 0.29162686 | −0.0098198 | 0.00751062 | 0.00296472 | 0.07017197 | −0.239278 | 0.12643716 | 0.23308518 |
| ITGB3 | | | | | | | | | |
| RSPO3)- | −0.0977699 | −0.0150876 | 0.10175971 | −0.1604572 | 0.10775365 | 0.08248238 | −0.062605 | −0.0220026 | 0.16884614 |
| LGR6 | | | | | | | | | |
| RSPO3)- | −0.1601608 | −0.0160754 | −0.0229938 | 0.08597404 | −0.2525941 | 0.02556163 | 0.15336474 | 0.33241107 | −0.2674572 |
| LGR4 | | | | | | | | | |
| RSPO3)- | −0.0062589 | −0.0909181 | −0.0173271 | 0.23328283 | −0.1135121 | −0.2268265 | 0.17615283 | 0.32740448 | −0.3035573 |
| SDC4 | | | | | | | | | |
| RSPO3)- | 0.04618375 | 0.09322397 | 0.26706868 | −0.3012056 | −0.2163169 | 0.00731273 | −0.1449705 | 0.06455863 | 0.14532279 |
| FZD8 | | | | | | | | | |
| ADAM15)- | 0.06653491 | 0.228722 | 0.24071146 | 0.40184453 | 0.32779974 | 0.11910408 | −0.0810277 | −0.0291173 | −0.1370224 |
| ITGB1 | | | | | | | | | |
| ADAM15)- | 0.09540438 | 0.14506885 | 0.12174715 | 0.43125371 | −0.1347256 | 0.170845 | −0.0036234 | −0.1303245 | −0.1819619 |
| ITGB3 | | | | | | | | | |
| CALM1)- | −0.1155582 | 0.20242448 | 0.21287114 | −0.136702 | 0.01581653 | −0.0137031 | 0.06458416 | 0.01791949 | 0.24211081 |
| KCNQ3 | | | | | | | | | |
| CALM1)- | −0.0838385 | 0.02147633 | 0.05938963 | 0.0801054 | 0.2324754 | 0.04361432 | −0.0346703 | −0.0149554 | −0.3366271 |
| CALCR | | | | | | | | | |
| CALM1)- | 0.02174056 | 0.28044402 | −0.2639955 | −0.1715415 | −0.1121582 | −0.1204256 | −0.0599012 | 0.16298297 | 0.06640316 |
| GRM4 | | | | | | | | | |
| CALM1)- | −0.0538885 | 0.14051846 | −0.0884866 | −0.0654809 | −0.209551 | −0.0351779 | 0.32187876 | 0.00764163 | 0.13768116 |
| CRHR1 | | | | | | | | | |
| CALM1)- | −0.2313647 | 0.31298791 | 0.16660628 | −0.242029 | 0.00988142 | 0.50909091 | 0.04677207 | −0.4252964 | 0.25823452 |
| MYLK | | | | | | | | | |
| CALM1)- | 0.0936131 | 0.07352021 | 0.1315018 | −0.0881423 | −0.0384742 | −0.0325428 | 0.1145003 | −0.0530962 | 0.13965744 |
| HMMR | | | | | | | | | |
| CALM1)- | 0.13709279 | 0.06936987 | −0.1808438 | −0.2375494 | 0.07321906 | 0.04255599 | 0.10072568 | −0.0291173 | 0.01304348 |
| SCN10A | | | | | | | | | |
| CALM1)- | 0.13133709 | −0.0191706 | −0.0732005 | 0.12055336 | −0.3373303 | −0.184136 | 0.18408223 | 0.02628632 | 0.09262187 |
| AQP6 | | | | | | | | | |
| CALM1)- | −0.0333366 | 0.23268224 | −0.0464143 | −0.2256917 | −0.0432835 | 0.21457277 | 0.10580407 | −0.1300481 | 0.29486166 |
| PTPRA | | | | | | | | | |
| CALM1)- | −0.1840822 | 0.12813334 | 0.21917628 | −0.0101449 | −0.0691905 | −0.3473334 | 0.04876281 | 0.26610008 | −0.0368906 |
| SCN4A | | | | | | | | | |
| COL9A1)- | −0.0245237 | 0.08003166 | 0.07100008 | −0.2267457 | −0.2744401 | −0.1527009 | −0.0030963 | −0.0056655 | −0.0247702 |
| ITGB1 | | | | | | | | | |
| COL9A1)- | 0.2474207 | 0.04357573 | 0.22936176 | −0.0351779 | −0.1914739 | −0.0920979 | −0.1439726 | 0.0567231 | 0.10922626 |
| MAG | | | | | | | | | |
| GIP)- GIPR | −0.06932 | 0.02299381 | 0.07309838 | −0.0196989 | 0.10946261 | −0.0805192 | 0.21511942 | −0.1907429 | 0.23472446 |
| GIP)- DPP4 | 0.10028002 | 0.29424167 | −0.0671194 | −0.2608295 | 0.22680077 | 0.04895243 | −0.0180638 | 0.03761776 | 0.16706743 |
| GIP)- | −0.0081697 | −0.0489524 | −0.0987841 | 0.09335573 | 0.16909391 | −0.0029647 | −0.0341328 | 0.1273428 | 0.12470767 |
| GPR84 | | | | | | | | | |
| APOA4)- | 0.00210845 | −0.0097516 | −0.0587731 | −0.0845906 | 0.08953159 | 0.11884841 | 0.0654809 | 0.11515152 | −0.1310935 |
| LDLR | | | | | | | | | |
| SEMA4F)- | 0.05186628 | −0.1200673 | 0.07616475 | 0.20739179 | −0.1090466 | 0.04889138 | −0.1113454 | −0.1618185 | −0.071805 |
| NRP2 | | | | | | | | | |
| GAD1)- | −0.2059763 | −0.2429334 | 0.16471131 | −0.0096182 | 0.01739761 | 0.12978457 | 0.21726525 | −0.0034257 | 0.23847167 |
| GRM4 | | | | | | | | | |
| ITIH2)- | −0.2288002 | −0.2748715 | 0.18977958 | 0.03946372 | 0.10635214 | 0.17193214 | −0.1797127 | 0.19392562 | −0.177608 |
| FCER1A | | | | | | | | | |
| CCL20)- | −0.0911038 | −0.0096208 | 0.10230719 | −0.0382082 | −0.0679429 | 0.08228203 | 0.21022803 | 0.05718427 | −0.0949307 |
| CCR6 | | | | | | | | | |
| FGF17)- | −0.0552956 | 0.09213128 | −0.213871 | −0.0163378 | 0.00115439 | 0.23737523 | −0.1129289 | 0.10139008 | −0.0891963 |
| FGFR2 | | | | | | | | | |
| FGF17)- | −0.086733 | 0.17490446 | 0.14974954 | −0.3322903 | −0.3478834 | 0.02108245 | 0.18487284 | −0.1133803 | 0.19868248 |
| FGFR1 | | | | | | | | | |
| COL4A2)- | −0.0912535 | −0.0301733 | −0.0913762 | −0.1682588 | −0.0897292 | 0.08137045 | −0.1226036 | −0.1019272 | 0.10033599 |
| ITGB3 | | | | | | | | | |
| LAMC3)- | −0.1529439 | −0.0506079 | −0.0608876 | 0.06173206 | 0.00210825 | 0.09869223 | 0.11185771 | 0.09275362 | −0.2367589 |
| ITGA6 | | | | | | | | | |
| LAMC3)- | 0.11979836 | −0.0415802 | −0.0177259 | 0.0756333 | 0.01640478 | −0.0544191 | −0.0150198 | −0.0312253 | 0.08155468 |
| ITGB1 | | | | | | | | | |
| CCL28)- | 0.05935637 | −0.0167353 | −0.0165436 | 0.00724733 | 0.00672269 | −0.15639 | 0.18533533 | −0.0202899 | 0.20911846 |
| CCR3 | | | | | | | | | |
| CXCL9)- | 0.17757133 | 0.14497529 | 0.05926366 | −0.1836331 | 0.14108692 | −0.0658805 | 0.05839513 | 0.05612648 | 0.06100936 |
| CCR3 | | | | | | | | | |
| TGM2)- | 0.21015185 | 0.17240357 | −0.2372055 | −0.3098258 | 0.13201517 | −0.2228664 | −0.0445528 | 0.19512516 | 0.2516469 |
| ITGA4 | | | | | | | | | |
| TGM2)- | −0.2302447 | 0.30402846 | 0.35791693 | −0.1384104 | −0.1241148 | −0.530584 | 0.18484848 | 0.51897233 | 0.2171278 |
| ITGB1 | | | | | | | | | |
| TGM2)- | −0.1393556 | −0.2023915 | 0.21108805 | −0.2742036 | −0.160029 | 0.11405416 | 0.08518348 | −0.0988964 | 0.4697938 |
| ITGB3 | | | | | | | | | |
| TGM2)- | 0.34935275 | −0.3039626 | 0.33999802 | −0.6911954 | 0.24052176 | 0.30192035 | −0.0778656 | −0.1550725 | 0.60724638 |
| SDC4 | | | | | | | | | |
| TGM2)- | −0.0658168 | 0.03010639 | −0.0911936 | 0.07042393 | −0.0890851 | −0.1618737 | 0.02339066 | 0.16799526 | 0.10316206 |
| TBXA2R | | | | | | | | | |
| TGM2)- | 0.26535788 | −0.1167364 | 0.26219572 | −0.4028459 | 0.09927863 | 0.18077012 | −0.1285903 | −0.0140975 | 0.39855072 |
| ADGRG1 | | | | | | | | | |
| CNTF)- | 0.06077226 | −0.3824809 | 0.00481992 | 0.35186771 | 0.18041645 | 0.04219132 | 0.06681162 | −0.1193145 | −0.0521739 |
| IL6R | | | | | | | | | |
| CNTF)- | −0.0800081 | −0.014919 | −0.0889257 | −0.1082416 | 0.25477665 | −0.148824 | 0.09131036 | −0.1106719 | 0.00474308 |
| IL6ST | | | | | | | | | |
| AGT)- | −0.0258269 | 0.24953887 | −0.2302468 | 0.20922266 | −0.1784726 | −0.1781526 | −0.0197863 | 0.2284886 | 0.26930171 |
| AGTR1 | | | | | | | | | |
| AGT)- | 0.00527044 | 0.25309618 | −0.1707655 | 0.26613966 | −0.2451376 | 0.09223269 | 0.15850207 | 0.02806509 | −0.0349144 |
| AGTR2 | | | | | | | | | |
| DLL1)- | −0.1143112 | 0.08020585 | 0.08223656 | −0.0190388 | −0.1161044 | 0.08431909 | 0.06384872 | 0.22023065 | 0.1428195 |
| NOTCH4 | | | | | | | | | |
| THBS2)- | 0.44163373 | 0.32529644 | −0.3980097 | −0.2447958 | 0.30106113 | −0.2258235 | −0.3655177 | 0.43649539 | 0.30764163 |
| ITGA4 | | | | | | | | | |
| THBS2)- | −0.1305665 | 0.10764163 | 0.22885375 | −0.0239789 | −0.0735178 | 0.72503294 | 0.20237154 | −0.213307 | 0.08221344 |
| ITGB1 | | | | | | | | | |
| THBS2)- | 0.27791139 | 0.19342513 | −0.0806377 | 0.20133079 | 0.07866131 | 0.12136386 | −0.033072 | 0.2646681 | 0.11667435 |
| ITGB3 | | | | | | | | | |
| THBS2)- | −0.0755848 | 0.32608696 | 0.0403914 | 0.17285903 | −0.1800152 | 0.14846788 | 0.02727902 | −0.0585173 | 0.36758893 |
| NOTCH4 | | | | | | | | | |
| THBS2)- | 0.55823452 | −0.3793149 | 0.51054018 | 0.06429513 | −0.2030303 | −0.2886693 | 0.52305665 | 0.56534914 | −0.4841897 |
| ITGA6 | | | | | | | | | |
| THBS2)- | 0.0212787 | 0.04176686 | 0.02123031 | 0.0024375 | −0.1515132 | 0.27247274 | −0.0226149 | −0.0289865 | −0.0111993 |
| CD36 | | | | | | | | | |
| TGFB2)- | 0.16816579 | −0.2304069 | −0.0492517 | −0.0089603 | 0.0266359 | −0.1023985 | −0.1659306 | −0.1270301 | 0.03537666 |
| ENG | | | | | | | | | |
| C4BPA)- | −0.1369466 | −0.0129887 | 0.0875944 | 0.25257749 | 0.01416431 | −0.0863034 | −0.167007 | −0.132288 | −0.1420995 |
| BMPR2 | | | | | | | | | |
| C4BPA)- | 0.23795874 | 0.44847382 | −0.2240027 | −0.1709542 | −0.0089606 | −0.0236511 | −0.2611761 | 0.30937479 | −0.1749728 |
| CD40 | | | | | | | | | |
| CFH)- | −0.1059323 | −0.3527125 | 0.21463175 | 0.19249012 | −0.1909972 | 0.26086957 | 0.10334147 | −0.1039526 | −0.0325428 |
| ITGAM | | | | | | | | | |
| PTGS2)- | −0.0061289 | −0.0697245 | −0.2166777 | 0.03162055 | 0.05409858 | 0.18880105 | 0.26133369 | −0.0023715 | 0.11910408 |
| ALOX5 | | | | | | | | | |
| FASLG)- | 0.00019819 | 0.00019818 | 0.02206959 | 0.0083004 | 0.11570521 | 0.18913666 | 0.10977498 | 0.25508087 | −0.0486166 |
| TNFRSF6B | | | | | | | | | |
| XCL1)- | 0.20007911 | 0.00395465 | 0.17371527 | 0.14341711 | 0.09666579 | −0.0633112 | 0.07439378 | 0.07918835 | 0.30989163 |
| ADGRV1 | | | | | | | | | |
| CCN2)- | −0.0351779 | −0.3097497 | 0.0180584 | 0.38853755 | −0.1563962 | 0.214361 | −0.0173334 | −0.0729908 | −0.2289855 |
| ITGAM | | | | | | | | | |
| CCN2)- | 0.07496706 | 0.09275362 | −0.1845617 | 0.17681159 | −0.0075775 | 0.18708827 | −0.2677824 | 0.18050066 | 0.19486166 |
| ITGB2 | | | | | | | | | |
| APOA2)- | −0.3325536 | 0.35429777 | −0.216519 | −0.1513834 | −0.0403162 | 0.09920949 | 0.04018578 | 0.23689845 | −0.4050858 |
| LDLR | | | | | | | | | |
| SEMA4A)- | −0.0033605 | −0.0895463 | −0.1136626 | −0.2434783 | 0.26666667 | 0.16350461 | −0.0295125 | −0.1683794 | 0.38102767 |
| PLXNB2 | | | | | | | | | |
| EFNA1)- | 0.04552342 | 0.21990908 | 0.18711276 | 0.34744227 | 0.00896477 | 0.12800158 | −0.058271 | −0.291841 | −0.3591027 |
| EPHB6 | | | | | | | | | |
| EFNA1)- | −0.0928378 | 0.19888666 | 0.08141412 | 0.17259552 | 0.05455983 | −0.2423983 | −0.1568925 | 0.15595454 | −0.1075099 |
| EPHB1 | | | | | | | | | |
| EFNA3)- | −0.0983558 | 0.16926423 | −0.0801161 | −0.0429541 | 0.077884 | 0.12737993 | 0.03388155 | −0.0617939 | 0.01607431 |
| EPHB6 | | | | | | | | | |
| EFNA3)- | −0.1498105 | −0.0144316 | 0.11409927 | 0.0747719 | 0.010741 | 0.011893 | −0.1902346 | 0.00270137 | 0.22318841 |
| EPHB1 | | | | | | | | | |
| ADAM12)- | −0.2211168 | 0.03090101 | 0.07787844 | −0.0918374 | −0.1280058 | 0.62164833 | 0.25335968 | −0.4063241 | 0.19025033 |
| ITGB1 | | | | | | | | | |
| ADAM12)- | 0.1835612 | −0.0890792 | 0.14989294 | 0.54081297 | −0.27123 | −0.2969893 | 0.49670619 | 0.53596838 | −0.3581028 |
| SDC4 | | | | | | | | | |
| S100A8)- | −0.1200778 | −0.168847 | 0.18941991 | 0.24492754 | −0.2542747 | 0.15757576 | −0.14233 | 0.17187654 | 0.22471096 |
| ITGB2 | | | | | | | | | |
| S100A8)- | −0.254325 | 0.08409399 | 0.29115719 | 0.04479578 | −0.1232128 | 0.05362319 | −0.0795967 | −0.095853 | −0.0878817 |
| TLR4 | | | | | | | | | |
| S100A8)- | −0.1192579 | −0.0847558 | 0.14534482 | 0.12418064 | −0.1841499 | −0.0880134 | −0.106221 | −0.0471046 | −0.0913762 |
| CD36 | | | | | | | | | |
| S100A8)- | 0.18087737 | −0.0187175 | −0.1592033 | 0.02332093 | −0.1485877 | 0.00507347 | −0.1044894 | −0.0571937 | 0.1492852 |
| AGER | | | | | | | | | |
| S100A9)- | −0.2416815 | −0.2652698 | 0.30573039 | 0.19380764 | −0.1821896 | 0.27681159 | −0.265015 | 0.2685112 | 0.22911726 |
| ITGB2 | | | | | | | | | |
| S100A9)- | −0.2203993 | −0.0509982 | 0.52412021 | 0.22885375 | −0.4581274 | 0.17496706 | −0.5525466 | 0.23399209 | 0.0428195 |
| TLR4 | | | | | | | | | |
| S100A9)- | 0.08292426 | 0.13501137 | 0.2406028 | −0.1517178 | −0.1716227 | 0.01363681 | −0.2546978 | −0.0296452 | −0.0113969 |
| CD36 | | | | | | | | | |
| S100A9)- | −0.0183538 | −0.3506737 | 0.13440809 | 0.17648803 | −0.0779195 | 0.24675496 | −0.1020469 | 0.20122554 | 0.32207912 |
| AGER | | | | | | | | | |
| WNT2B)- | −0.1286773 | 0.01153023 | 0.205895 | 0.07325669 | 0.06253504 | −0.0572483 | 0.05920556 | −0.1443394 | −0.0306993 |
| FZD4 | | | | | | | | | |
| L1CAM)- | −0.0866654 | 0.05733331 | −0.2697364 | −0.256069 | −0.0135229 | 0.1805185 | −0.0527115 | 0.12024774 | −0.2137304 |
| FGFR2 | | | | | | | | | |
| L1CAM)- | −0.1279693 | 0.15200915 | 0.01544199 | 0.22932244 | −0.0269504 | 0.10511579 | 0.04487792 | 0.01284839 | −0.0698379 |
| EPHB2 | | | | | | | | | |
| L1CAM)- | 0.02513693 | −0.0874185 | −0.0510427 | −0.0228598 | −0.2034066 | 0.21114002 | 0.17627677 | −0.2630123 | 0.20094874 |
| CNTN1 | | | | | | | | | |
| COL11A1)- | −0.3329381 | −0.0056653 | 0.0911726 | 0.00540184 | −0.0393939 | 0.72582345 | 0.17206851 | −0.5241107 | 0.07536232 |
| ITGB1 | | | | | | | | | |
| COL11A1)- | 0.19578393 | −0.0537549 | 0.19802372 | 0.04940711 | −0.1150198 | −0.2155468 | 0.26772069 | 0.34519104 | −0.197892 |
| DDR1 | | | | | | | | | |
| CD40LG)- | −0.1790268 | 0.0023062 | 0.18270214 | 0.10191377 | −0.1113198 | 0.07668237 | −0.2037172 | 0.11620553 | 0.23109354 |
| ITGAM | | | | | | | | | |
| CD40LG)- | 0.34396416 | −0.354759 | 0.13410656 | 0.29730887 | −0.2045791 | 0.07437908 | −0.2186059 | 0.19019072 | 0.1115942 |
| CD40 | | | | | | | | | |
| CD40LG)- | 0.05455804 | 0.00929068 | −0.0818889 | 0.05948813 | 0.02016342 | −0.0011858 | −0.120054 | 0.14914361 | 0.14980237 |
| ITGB2 | | | | | | | | | |
| CD40LG)- | −0.1094527 | −0.2107864 | 0.12465392 | 0.11377186 | −0.1734331 | 0.18384557 | −0.0294593 | 0.25021411 | 0.11185771 |
| TRAF3 | | | | | | | | | |
| BMP5)- | −0.1665294 | −0.0823723 | 0.05766253 | 0.0557349 | −0.0504661 | 0.08485687 | −0.0030304 | −0.0711486 | 0.13478261 |
| BMPR2 | | | | | | | | | |
| BMP5)- | 0.02985271 | −0.0330148 | −0.0220566 | 0.11555438 | −0.184482 | 0.32615212 | −0.1129825 | 0.21199644 | 0.32094862 |
| BMPR1A | | | | | | | | | |
| BMP5)- | 0.12060898 | −0.0510708 | 0.1529889 | 0.06798867 | 0.06328907 | 0.04434341 | −0.0806882 | 0.31901437 | 0.10961792 |
| BMPR1B | | | | | | | | | |
| ITGB3BP)- | 0.05049939 | 0.08437707 | 0.22214901 | −0.0525761 | 0.13559099 | −0.0517906 | 0.00230582 | 0.05040356 | 0.27636867 |
| ITGB3 | | | | | | | | | |
| GNAS)- | 0.03860343 | −0.0067194 | −0.1325112 | 0.17280453 | −0.096471 | 0.17840437 | −0.1863995 | −0.341502 | −0.3472991 |
| CRHR1 | | | | | | | | | |
| GNAS)- | −0.0747036 | −0.0193682 | −0.1142763 | −0.1810126 | 0.15507129 | −0.4916002 | −0.3088033 | 0.29459816 | 0.11291545 |
| GCGR | | | | | | | | | |
| GNAS)- | −0.0640996 | 0.02305665 | −0.0330301 | −0.0624547 | 0.20214946 | 0.10883816 | −0.1716773 | 0.04512665 | 0.19209486 |
| ADCY7 | | | | | | | | | |
| RBP4)- | −0.0996869 | −0.0081765 | −0.0874139 | 0.06496245 | −0.1866763 | 0.314361 | 0.06589352 | −0.1591568 | 0.13400975 |
| STRA6 | | | | | | | | | |
| COL5A1)- | −0.3716733 | 0.17918314 | 0.28985507 | −0.1000033 | −0.1588326 | 0.86228137 | 0.35072464 | −0.3470356 | 0.22924901 |
| ITGB1 | | | | | | | | | |
| CEL)- | −0.185835 | −0.1973095 | 0.0896891 | 0.10289855 | 0.07694588 | 0.14611331 | 0.03524606 | 0.21496097 | 0.18136302 |
| CXCR4 | | | | | | | | | |
| MMP9)- | −0.1389603 | 0.06061804 | 0.15375742 | 0.10377203 | −0.1058304 | 0.0730028 | −0.1496738 | −0.029249 | 0.13741765 |
| ITGAM | | | | | | | | | |
| MMP9)- | 0.30502109 | 0.00013178 | −0.0901691 | −0.1095042 | 0.04979568 | −0.2033407 | −0.1888036 | −0.1121945 | −0.1524374 |
| RECK | | | | | | | | | |
| MMP9)- | −0.1638664 | −0.2453054 | −0.0009883 | −0.0099489 | 0.14080053 | 0.19594795 | 0.16403162 | 0.22938076 | −0.1606061 |
| CD44 | | | | | | | | | |
| MMP9)- | −0.0076431 | 0.04348686 | −0.0416516 | 0.13776973 | −0.1905562 | 0.16741888 | −0.1154416 | 0.21449275 | 0.15783926 |
| ITGB2 | | | | | | | | | |
| MMP9)- | 0.19438587 | 0.07886934 | −0.1038323 | −0.2273761 | 0.10863978 | −0.1630811 | −0.0491549 | 0.12556822 | −0.3142292 |
| EPHB2 | | | | | | | | | |
| SPTAN1)- | 0.07056033 | 0.02562667 | 0.27624601 | −0.0555354 | −0.0359061 | 0.16589255 | −0.078266 | 0.25956914 | −0.2575758 |
| PTPRA | | | | | | | | | |
| COL9A2)- | 0.3279747 | −0.052642 | 0.01021215 | 0.30223979 | 0.1942029 | −0.1011858 | −0.0736495 | 0.13873518 | 0.03346509 |
| ITGB1 | | | | | | | | | |
| COL9A2)- | 0.15990776 | −0.1392147 | −0.0099176 | −0.0491436 | 0.1564209 | −0.1042854 | −0.1068722 | 0.16680391 | 0.1057971 |
| MAG | | | | | | | | | |
| PLAU)- | −0.3326854 | −0.3987744 | 0.28913281 | 0.41654863 | −0.1984512 | 0.16634277 | 0.23522048 | −0.2799736 | −0.1662714 |
| ITGAM | | | | | | | | | |
| PLAU)- | −0.1831779 | 0.01238757 | 0.08730603 | −0.1924306 | −0.2199018 | 0.44672091 | 0.22411067 | −0.1334651 | 0.13241107 |
| ITGB1 | | | | | | | | | |
| PLAU)- | 0.14858499 | 0.20340659 | −0.1675674 | −0.0832043 | 0.0863864 | 0.01673309 | −0.379007 | 0.33491436 | 0.18656126 |
| ITGB2 | | | | | | | | | |
| ANGPTL2)- | −0.1469987 | −0.0757042 | −0.1475734 | −0.1154847 | 0.30492506 | −0.1832137 | 0.08630913 | −0.1149577 | 0.03741765 |
| LILRB2 | | | | | | | | | |
| GHRH)- | −0.0956525 | −0.0828637 | 0.13230253 | 0.00322804 | −0.1739589 | 0.32036628 | −0.0736006 | 0.05559947 | 0.19183136 |
| GPR84 | | | | | | | | | |
| COL16A1)- | 0.05217391 | 0.2458498 | 0.31699605 | −0.0943347 | −0.1495389 | −0.2383399 | 0.27061924 | 0.05981555 | 0.22714097 |
| ITGB1 | | | | | | | | | |
| TNFSF15)- | 0.10331763 | 0.04164333 | 0.01542671 | 0.11870883 | −0.1391379 | −0.1188406 | 0.30707887 | −0.0054018 | −0.1620553 |
| TNFRSF25 | | | | | | | | | |
| HSPG2)- | 0.09631094 | 0.09947299 | 0.23438735 | −0.2604743 | −0.0678524 | 0.22055336 | 0.42002635 | 0.40500659 | −0.0346509 |
| SDC1 | | | | | | | | | |
| HSPG2)- | −0.001054 | −0.0516469 | 0.10131752 | −0.1065876 | −0.1238472 | 0.27852437 | −0.1542819 | 0.35151515 | −0.2546772 |
| ITGB1 | | | | | | | | | |
| HSPG2)- | −0.1103498 | −0.3413702 | 0.02589871 | 0.1 | 0.01120301 | 0.15244746 | −0.2313091 | 0.11160156 | −0.285112 |
| COL13A1 | | | | | | | | | |
| HSPG2)- | 0.13452797 | 0.27615283 | 0.28027408 | 0.05928854 | 0.00065885 | 0.21094934 | 0.3236263 | 0.0965808 | 0.13227931 |
| FGFR1 | | | | | | | | | |
| COL5A2)- | −0.4339921 | 0.23201581 | 0.37048748 | −0.1653491 | −0.1940711 | 0.83781291 | 0.37022398 | −0.4557312 | 0.23596838 |
| ITGB1 | | | | | | | | | |
| COL5A2)- | 0.42964427 | −0.1110672 | 0.28656126 | 0.0770751 | −0.2003953 | −0.2300395 | 0.30790514 | 0.49525692 | −0.1789196 |
| DDR1 | | | | | | | | | |
| F10)- | −0.2002914 | −0.0539043 | 0.23481657 | 0.22819471 | −0.2993303 | 0.22028044 | −0.2082057 | 0.1075793 | 0.1768833 |
| ITGAM | | | | | | | | | |
| F10)- | 0.02391342 | −0.0233849 | −0.0552052 | 0.17596055 | −0.1243156 | 0.15815344 | −0.2172509 | 0.20297111 | 0.13090023 |
| ITGB2 | | | | | | | | | |
| SYK)- LAT | 0.11063157 | 0.10726758 | −0.0357237 | −0.1002635 | 0.19490814 | 0.08043743 | 0.02414091 | 0.02147633 | −0.3054018 |
| FGF14)- | −0.0234883 | −0.1179615 | 0.22028076 | −0.0840225 | 0.1088786 | 0.09437507 | 0.21286298 | −0.0166019 | −0.2218709 |
| FGFR2 | | | | | | | | | |
| FGF14)- | −0.1596676 | 0.13214595 | 0.03246349 | −0.2121322 | −0.0959631 | 0.19744951 | 0.30820925 | −0.2187232 | 0.3259552 |
| FGFR1 | | | | | | | | | |
| HLA-C)- | −0.0072469 | −0.005929 | 0.03310822 | 0.13504611 | −0.2027276 | 0.15725156 | −0.0201608 | −0.1061959 | −0.1079051 |
| LILRB2 | | | | | | | | | |
| HLA-C)- | −0.2209559 | −0.3185876 | 0.37307107 | −0.1061924 | 0.01358616 | −0.0051383 | −0.1222754 | 0.13333333 | 0.09565217 |
| CD3G | | | | | | | | | |
| HLA-C)- | 0.0459861 | 0.27458085 | 0.26731467 | 0.10527009 | −0.1656618 | −0.0111338 | −0.1553161 | −0.1832795 | 0.11633729 |
| KIR3DL1 | | | | | | | | | |
| HLA-E)- | −0.1674244 | −0.0940208 | 0.28960666 | 0.23926219 | −0.1188577 | 0.16601337 | −0.1571797 | 0.1659475 | 0.16442688 |
| KLRC2 | | | | | | | | | |
| HLA-E)- | −0.4178554 | −0.5628727 | 0.555691 | −0.2670619 | 0.35955204 | −0.1488801 | 0.18708827 | −0.1197628 | 0.03662714 |
| KLRD1 | | | | | | | | | |
| HLA-E)- | 0.04737588 | 0.45409324 | 0.28376063 | 0.29855072 | 0.19900498 | −0.0743132 | 0.22299102 | −0.1810396 | 0.20131752 |
| KIR3DL1 | | | | | | | | | |
| ANXA1)- | 0.29920949 | 0.25362319 | −0.1720763 | 0.31370224 | −0.1725377 | 0.26956522 | 0.13563121 | −0.2617918 | −0.2895916 |
| FPR1 | | | | | | | | | |
| ANXA1)- | 0.16950047 | −0.0340591 | 0.26152582 | −0.0010541 | 0.27523483 | 0.11724002 | −0.272928 | −0.0119942 | −0.078527 |
| DYSF | | | | | | | | | |
| IL31)- | 0.17378983 | 0.08262775 | −0.0928569 | −0.271278 | 0.01041392 | −0.2519602 | 0.20491698 | −0.158134 | −0.016996 |
| IL31RA | | | | | | | | | |
| ACTR2)- | 0.2676989 | −0.3211991 | 0.29056169 | −0.0204216 | 0.17641634 | 0.15428195 | −0.1067194 | −0.1583663 | 0.40382082 |
| LDLR | | | | | | | | | |
| NDP)- | −0.2180632 | −0.1287028 | −0.0175103 | −0.1139036 | −0.1813747 | 0.29381732 | 0.43181156 | 0.01139695 | 0.07819757 |
| FZD4 | | | | | | | | | |
| NDP)- | −0.0802003 | −0.187881 | 0.12442336 | 0.13906914 | −0.1721457 | 0.10804045 | 0.14823111 | 0.20369577 | −0.0320827 |
| LGR4 | | | | | | | | | |
| CCL7)- | −0.2923389 | 0.06596963 | −0.1727711 | 0.16722122 | 0.26370949 | 0.15000494 | 0.17406494 | 0.16469038 | 0.40018448 |
| CCR3 | | | | | | | | | |
| BMP2)- | −0.1346116 | −0.0598195 | 0.08926395 | 0.0164037 | −0.0438065 | 0.03472245 | 0.21829573 | −0.1817343 | 0.00685202 |
| BMPR1B | | | | | | | | | |
| AGRN)- | −0.5921144 | 0.05395435 | 0.16285121 | 0.08155468 | 0.1397892 | −0.5881423 | −0.0816864 | 0.62305665 | −0.0557312 |
| ITGB1 | | | | | | | | | |
| AGRN)- | −0.1126928 | 0.08669587 | −0.1543582 | 0.06837945 | −0.2082647 | −0.207651 | 0.2584855 | 0.32521667 | 0.27891963 |
| ATP1A3 | | | | | | | | | |
| INSL3)- | −0.1928426 | −0.0769789 | 0.13299714 | −0.0291173 | −0.0282015 | 0.15981555 | 0.03716272 | −0.0400527 | −0.0061924 |
| GPR84 | | | | | | | | | |
| INSL3)- | −0.0608358 | 0.27502804 | −0.0512296 | −0.1611331 | −0.0054699 | 0.05823836 | 0.17813366 | −0.1283352 | 0.03596838 |
| RXFP1 | | | | | | | | | |
| UCN3)- | −0.1409602 | −0.2072556 | 0.18864446 | 0.05323495 | −0.1263936 | −0.0500725 | 0.21707743 | −0.0608716 | 0.0589611 |
| CRHR1 | | | | | | | | | |
| SHBG)- | −0.2023531 | 0.12648718 | 0.11818214 | −0.3525808 | 0.06106921 | 0.31937811 | 0.17496706 | 0.15085639 | −0.1180501 |
| CLDN4 | | | | | | | | | |
| ZG16B)- | 0.10298817 | −0.2256194 | 0.20741357 | −0.2062059 | 0.2883548 | 0.13419414 | −0.305997 | 0.1637843 | 0.17123468 |
| TLR5 | | | | | | | | | |
| ZG16B)- | −0.1252553 | −0.207485 | −0.1195269 | −0.3268775 | −0.0806351 | −0.2700922 | 0.18862206 | 0.17800909 | 0.27116411 |
| CXCR4 | | | | | | | | | |
| RPH3A)- | −0.0041853 | 0.06083375 | 0.06647542 | 0.04242704 | −0.0605142 | 0.02991073 | −0.0817376 | 0.07022861 | −0.070951 |
| NRXN1 | | | | | | | | | |
| LAMA1)- | 0.13257776 | −0.1858197 | 0.12565894 | 0.01153061 | −0.0794623 | −0.1278909 | −0.0492754 | 0.01198946 | −0.1061924 |
| ITGA6 | | | | | | | | | |
| LAMA1)- | −0.1098188 | 0.17811018 | 0.3199895 | 0.2264611 | 0.17267875 | −0.1498419 | −0.0657348 | 0.03070031 | 0.16429513 |
| ITGA7 | | | | | | | | | |
| LAMA1)- | −0.3313126 | −0.1254613 | −0.064378 | −0.1298676 | −0.1444291 | 0.26507215 | −0.1926219 | 0.01554677 | −0.0965744 |
| ITGB1 | | | | | | | | | |
| LAMB3)- | 0.11831357 | −0.1453228 | 0.0198946 | −0.2849802 | 0.18880105 | 0.24361001 | −0.359025 | −0.3507246 | 0.46758893 |
| CD151 | | | | | | | | | |
| LAMB3)- | −0.2805007 | 0.16389987 | 0.30500659 | 0.06640316 | 0.17549407 | −0.3097497 | 0.1773386 | 0.44097497 | 0.31725955 |
| ITGB1 | | | | | | | | | |
| LAMB3)- | 0.45256917 | −0.3075099 | 0.51225296 | −0.3951252 | 0.46916996 | 0.42068511 | −0.1963109 | −0.215415 | 0.34664032 |
| ITGA6 | | | | | | | | | |
| AFDN)- | −0.1617366 | 0.06087358 | −0.1077126 | 0.16054547 | −0.0417916 | −0.1772786 | −0.0970007 | 0.16483299 | −0.174188 |
| EPHB6 | | | | | | | | | |
| AFDN)- | −0.3600909 | 0.14704042 | −0.4088438 | 0.09894598 | −0.422603 | −0.3039526 | 0.17865499 | 0.19052011 | −0.2164762 |
| NECTIN3 | | | | | | | | | |
| IL1F10)- | 0.31152566 | 0.06128501 | −0.0464656 | 0.26331566 | 0.05145945 | −0.2172652 | −0.0310328 | 0.07815229 | −0.0552042 |
| IL1R1 | | | | | | | | | |
| CCL8)- | 0.0777441 | 0.02095414 | −0.0992716 | 0.19819464 | 0.0292654 | 0.07200738 | 0.05048608 | 0.14703557 | 0.17235473 |
| CCR3 | | | | | | | | | |
| SPP1)- | −0.0272077 | 0.04670773 | −0.0211567 | 0.18484848 | −0.2205892 | 0.23254282 | −0.1074277 | 0.17615283 | 0.10210804 |
| ITGA4 | | | | | | | | | |
| SPP1)- | −0.1259594 | 0.11983267 | −0.0542837 | −0.1438735 | 0.02885375 | 0.21870883 | 0.17957839 | 0.25783926 | −0.2916996 |
| CD44 | | | | | | | | | |
| SPP1)- | 0.35073619 | 0.0267466 | −0.0082348 | 0.11752306 | 0.13399209 | −0.0657444 | 0.16376812 | −0.2832675 | 0.12463768 |
| ITGB1 | | | | | | | | | |
| SPP1)- | −0.0509982 | 0.16819844 | 0.32987449 | −0.1225377 | −0.2855919 | 0.21439631 | −0.2832202 | 0.09164882 | −0.1299163 |
| ITGB3 | | | | | | | | | |
| ADM2)- | −0.0447387 | −0.4169989 | 0.15319976 | −0.1457181 | −0.3277652 | 0.25587141 | 0.22405273 | 0.15455564 | 0.18696268 |
| CALCRL | | | | | | | | | |
| ADM2)- | −0.4349201 | −0.3690331 | 0.28624621 | 0.08418972 | 0.15642606 | 0.07918314 | 0.27909199 | −0.0791857 | −0.0399881 |
| GPR84 | | | | | | | | | |
| CXCL12)- | −0.1940865 | 0.13218186 | 0.05518016 | 0.12978457 | 0.122933 | 0.18486066 | −0.0915679 | 0.16047431 | −0.0874835 |
| ITGB1 | | | | | | | | | |
| CXCL12)- | 0.17529754 | 0.18281312 | −0.160008 | −0.33296 | 0.46572454 | −0.2500824 | 0.40989492 | −0.2354414 | −0.2137022 |
| CXCR4 | | | | | | | | | |
| BMP7)- | 0.02004021 | 0.06578124 | −0.0066948 | 0.15112487 | −0.1799394 | 0.14020754 | −0.2937571 | 0.17334871 | −0.135907 |
| BMPR1B | | | | | | | | | |
| COL4A3)- | 0.2421945 | −0.0067251 | 0.10898664 | −0.1962309 | −0.2124078 | −0.1099212 | −0.1907899 | 0.01159611 | 0.04308584 |
| ITGB3 | | | | | | | | | |
| COL4A4)- | 0.43293007 | −0.0745004 | −0.1180002 | −0.1218315 | 0.05260904 | −0.3035538 | 0.04302138 | 0.18804771 | 0.0345884 |
| ITGB3 | | | | | | | | | |
| CNTN4)- | −0.0149082 | 0.10130931 | 0.05382235 | 0.03333333 | 0.02332093 | −0.008565 | 0.02740538 | −0.1404665 | −0.0424242 |
| PTPRG | | | | | | | | | |
| TCTN1)- | 0.32222341 | −0.0523175 | 0.10355177 | 0.24831994 | −0.2157522 | 0.17736781 | −0.221695 | −0.0384083 | 0.01758951 |
| TMEM67 | | | | | | | | | |
| PDCD1LG2)- | 0.08648289 | 0.12822936 | −0.0933755 | −0.1827404 | −0.1006886 | −0.0287898 | 0.03874667 | −0.2392121 | −0.226087 |
| PDCD2 | | | | | | | | | |
| NPS)- | 0.23764781 | −0.1050931 | 0.01868607 | 0.0770751 | 0.127961 | −0.241502 | −0.1259843 | 0.09393939 | 0.35072464 |
| GPR84 | | | | | | | | | |
| TNFSF11)- | 0.21670074 | −0.031734 | 0.25764568 | −0.0443793 | −0.1851071 | 0.06589352 | 0.11114405 | 0.26423795 | −0.1943411 |
| TNFRSF11A | | | | | | | | | |
| POMC)- | −0.1496688 | −0.0347316 | 0.18906358 | −0.1918693 | −0.1754103 | 0.31521382 | 0.26163883 | 0.05237327 | 0.11844922 |
| MC1R | | | | | | | | | |
| POMC)- | 0.06069793 | 0.34191192 | 0.10863547 | −0.1397509 | 0.08396217 | 0.04678131 | 0.30996311 | −0.2276755 | −0.0276689 |
| GPR84 | | | | | | | | | |
| FBLN2)- | −0.1760074 | −0.1624168 | 0.178955 | 0.29970023 | 0.04743552 | 0.35099164 | 0.1181896 | −0.1171471 | −0.1672047 |
| ITGB3 | | | | | | | | | |
| MDK)- | 0.1055336 | −0.3474308 | 0.22226614 | −0.1990777 | 0.11172596 | −0.0454545 | −0.3351889 | −0.2130505 | 0.15652689 |
| ITGA6 | | | | | | | | | |
| GCG)- | −0.1821117 | −0.0135727 | 0.08590352 | 0.10304388 | 0.0101486 | 0.15667413 | 0.02523639 | 0.16666667 | 0.12661397 |
| GPR84 | | | | | | | | | |
| PTPN6)- | 0.14957828 | 0.11003492 | −0.1487721 | −0.0396588 | −0.0498466 | −0.0858122 | −0.0985709 | −0.0435484 | −0.0295794 |
| CD300LF | | | | | | | | | |
| APLN)- | −0.0273539 | −0.3993673 | 0.18827486 | 0.37250058 | −0.349186 | 0.1978127 | −0.0573425 | 0.07549656 | 0.1011891 |
| APLNR | | | | | | | | | |
| CCN1)- | −0.0075099 | −0.1811594 | −0.0678837 | 0.41146245 | −0.1340539 | 0.19525692 | −0.1380083 | 0.05230567 | −0.0972332 |
| ITGAM | | | | | | | | | |
| CCN1)- | −0.0430901 | −0.0003953 | −0.0977667 | 0.12102247 | 0.18993346 | 0.20833471 | −0.1180578 | 0.00929007 | −0.0193689 |
| ITGB3 | | | | | | | | | |
| HLA-B)- | −0.3154254 | −0.3703679 | 0.39170319 | −0.2314888 | 0.17338841 | −0.1111989 | 0.10664475 | −0.014888 | −0.1075099 |
| CD3G | | | | | | | | | |
| LAMA2)- | 0.15568586 | 0.23407339 | −0.2016023 | −0.1513834 | 0.14650233 | −0.1260953 | −0.2158635 | 0.31089005 | 0.10658762 |
| ITGA7 | | | | | | | | | |
| CCL24)- | 0.04038607 | 0.02095345 | 0.03137565 | 0.1020926 | 0.00372519 | −0.0424396 | −0.121338 | −0.0619236 | 0.17261826 |
| CCR3 | | | | | | | | | |
| HRAS)- | 0.01713401 | −0.0853293 | 0.24567086 | −0.24146 | −0.0757561 | 0.12216255 | 0.08290469 | 0.04664646 | 0.14650856 |
| AGTR1 | | | | | | | | | |
| ICAM2)- | 0.03785908 | −0.0631205 | 0.00758851 | 0.01225458 | 0.10269594 | −0.1838187 | −0.0543729 | 0.21383399 | 0.31040843 |
| ITGAM | | | | | | | | | |
| FSHB)- | 0.12967844 | 0.02451239 | 0.15748888 | −0.0094218 | 0.11569131 | −0.1011366 | 0.1873291 | −0.0959157 | −0.0519104 |
| GPR84 | | | | | | | | | |
| RARRES2)- | −0.1577075 | −0.0492819 | −0.0862944 | 0.20162087 | 0.16313954 | 0.27030766 | −0.1053465 | −0.0923584 | −0.1290025 |
| GPR1 | | | | | | | | | |
| LRPAP1)- | −0.0236573 | 0.07327842 | 0.03288303 | −0.0179848 | 0.17767384 | 0.12147963 | −0.0650219 | −0.090978 | 0.02852531 |
| LDLR | | | | | | | | | |
| HSPA8)- | 0.2191113 | −0.0529662 | 0.27701835 | 0.11357049 | 0.01976285 | 0.03992095 | −0.1347826 | −0.1306983 | 0.12885375 |
| LDLR | | | | | | | | | |
| CALCB)- | −0.0193714 | −0.1482506 | −0.1167496 | 0.04487201 | 0.32597377 | 0.12189899 | 0.26633282 | −0.1332016 | 0.12055336 |
| GPR84 | | | | | | | | | |
| ADM)- | 0.07979442 | 0.0959378 | −0.1091742 | 0.01936886 | 0.35086159 | 0.03854009 | 0.15573933 | 0.09045094 | −0.0088277 |
| GPR84 | | | | | | | | | |
| PTHLH)- | 0.10541934 | 0.01054853 | 0.20755716 | 0.11792608 | 0.12790353 | 0.08096713 | 0.04632162 | 0.14953887 | 0.15098814 |
| GPR84 | | | | | | | | | |
| S100A10)- | 0.13465086 | −0.0581028 | 0.31493626 | −0.0498024 | 0.29885665 | 0.12213439 | −0.2552967 | −0.184058 | 0.18089592 |
| CFTR | | | | | | | | | |
| SHANK2)- | 0.02675541 | 0.14326667 | −0.2009691 | −0.3077176 | −0.0064914 | 0.03050166 | 0.08296814 | −0.1213439 | 0.15599473 |
| CFTR | | | | | | | | | |
| HSP90AA1)- | −0.0147563 | 0.00764163 | 0.1882105 | −0.0295125 | 0.1945369 | −0.0071146 | −0.2196448 | −0.0715415 | 0.2372859 |
| CFTR | | | | | | | | | |
| PLAU)- | 0.32649162 | −0.129806 | 0.39429382 | 0.39197602 | −0.3946112 | −0.3157548 | 0.3685112 | 0.36758893 | 0.20948617 |
| ITGA3 | | | | | | | | | |
| LAMA3)- | 0.18524374 | 0.01264822 | 0.38814229 | −0.0222661 | 0.27549407 | 0.12503294 | −0.2974967 | −0.0732543 | 0.23754941 |
| ITGA3 | | | | | | | | | |
| LAMA4)- | 0.34875984 | 0.00105405 | 0.39678514 | 0.3929501 | −0.5676824 | −0.5091748 | 0.42371542 | 0.34848485 | 0.15388669 |
| ITGA3 | | | | | | | | | |
| LAMA2)- | 0.19302984 | −0.0212794 | 0.24481191 | −0.0660079 | −0.0894598 | −0.0766798 | 0.23254282 | 0.22635046 | 0.08893281 |
| ITGA3 | | | | | | | | | |
| LAMA1)- | 0.28630734 | −0.1454929 | 0.18035056 | 0.10140344 | −0.1515451 | −0.2677077 | −0.0791831 | −0.0632411 | −0.0719368 |
| ITGA3 | | | | | | | | | |
| LGALS8)- | 0.08986026 | 0.10001322 | 0.07311447 | −0.1312729 | −0.0161455 | −0.0346634 | −0.0313581 | 0.03906585 | −0.1562634 |
| ITGA3 | | | | | | | | | |
| NID1)- | 0.38202839 | −0.1349188 | 0.51885767 | 0.19617918 | −0.4665349 | −0.2247694 | 0.49011858 | 0.28577075 | −0.1947299 |
| ITGA3 | | | | | | | | | |
| LAMC3)- | 0.11742612 | −0.0532437 | 0.1605878 | −0.0206872 | −0.0805745 | 0.01271535 | −0.2504611 | −0.2328063 | 0.19380764 |
| ITGA3 | | | | | | | | | |
| THBS1)- | 0.28405797 | 0.0916996 | 0.28827404 | −0.0752306 | −0.2864295 | −0.3094862 | 0.31238472 | 0.25270092 | 0.13860343 |
| ITGA3 | | | | | | | | | |
| CALR)- | 0.04012122 | 0.14875815 | 0.22353251 | 0.24716733 | −0.0620553 | −0.0069829 | 0.28787879 | 0.24163373 | −0.1043478 |
| ITGA3 | | | | | | | | | |
| TIMP2)- | 0.29209486 | 0.05059289 | 0.44795784 | 0.62674572 | −0.5803689 | −0.5873518 | 0.46890646 | 0.28972332 | 0.18629776 |
| ITGA3 | | | | | | | | | |
| LAMB2)- | 0.28906456 | −0.1054018 | 0.34400527 | 0.07510624 | −0.3773759 | −0.4084725 | 0.23596838 | 0.16284585 | 0.03636364 |
| ITGA3 | | | | | | | | | |
| COL4A3)- | 0.09210182 | −0.1451741 | 0.13205437 | −0.0204915 | 0.03538249 | 0.01311195 | 0.03952569 | 0.13491436 | −0.3051383 |
| ITGA3 | | | | | | | | | |
| LAMA5)- | 0.46002833 | −0.3760993 | 0.47597088 | −0.6252059 | 0.45569537 | 0.51419725 | −0.214968 | −0.3542394 | 0.77198762 |
| ITGA3 | | | | | | | | | |
| ADAM9)- | 0.24848485 | 0.04084321 | 0.39183136 | −0.0366271 | 0.41185771 | 0.34110672 | −0.340448 | −0.2992095 | 0.23952569 |
| ITGA3 | | | | | | | | | |
| LAMC1)- | 0.35880881 | −0.2222954 | 0.31242588 | 0.51549788 | −0.5308475 | −0.4795942 | 0.3657444 | 0.39314888 | −0.3262187 |
| ITGA3 | | | | | | | | | |
| LAMB3)- | 0.48379447 | −0.142556 | 0.57747036 | −0.1737813 | 0.53254282 | 0.53583663 | −0.3050066 | −0.3387352 | 0.51001318 |
| ITGA3 | | | | | | | | | |
| COL18A1)- | 0.26043693 | 0.01304821 | 0.3890079 | 0.49764485 | −0.2278072 | −0.2054086 | 0.19947299 | 0.16060606 | 0.0088274 |
| ITGA3 | | | | | | | | | |
| VTN)- | 0.12296438 | 0.06481179 | 0.14986489 | −0.218086 | 0.07702191 | −0.0432219 | 0.27536232 | 0.32911726 | −0.0703557 |
| ITGA3 | | | | | | | | | |
| LAMB1)- | 0.514361 | −0.2907773 | 0.42938076 | 0.46918541 | −0.2367667 | −0.3030403 | 0.3171278 | 0.48722003 | −0.3736495 |
| ITGA3 | | | | | | | | | |
| LAMC2)- | 0.46324111 | −0.0797101 | 0.54097497 | −0.1442688 | 0.63386034 | 0.59064559 | −0.4434783 | −0.4894598 | 0.75480896 |
| ITGA3 | | | | | | | | | |
| FN1)- | 0.55915679 | −0.1998682 | 0.55783926 | 0.72819499 | −0.5160738 | −0.5876153 | 0.5198946 | 0.49670619 | −0.0424242 |
| ITGA3 | | | | | | | | | |
| MIF)- CD74 | 0.07873497 | 0.05745347 | 0.06595289 | 0.09934778 | 0.0692404 | 0.01712893 | 0.05046113 | −0.0011858 | −0.1612648 |
| APP)- CD74 | 0.15870088 | 0.02832768 | −0.0971705 | −0.243083 | 0.31791831 | −0.1671937 | −0.0080369 | −0.1756258 | −0.1214756 |
| HLA-G)- | 0.16628236 | 0.08024508 | 0.19948597 | 0.11284957 | 0.0276771 | 0.17338603 | 0.09113674 | 0.09947299 | 0.05665536 |
| CD4 | | | | | | | | | |
| CXCL12)- | 0.17589088 | 0.08471785 | 0.06835494 | 0.09240043 | −0.0454396 | 0.10830753 | −0.0721582 | 0.17852437 | 0.20955894 |
| CD4 | | | | | | | | | |
| SLPI)- CD4 | 0.33675889 | 0.40633749 | −0.2826359 | 0.37135611 | −0.3385173 | 0.28458498 | 0.19828667 | −0.3666667 | −0.362199 |
| ADCYAP1)- | 0.11456995 | −0.0001318 | 0.24970341 | −0.202128 | 0.1561429 | 0.16964786 | 0.13194057 | −0.0176548 | 0.0911726 |
| SCTR | | | | | | | | | |
| SCT)- | −0.1215237 | 0.43495456 | −0.1916598 | −0.0983563 | 0.25693668 | 0.06785467 | −0.1856526 | −0.0599473 | 0.28247694 |
| SCTR | | | | | | | | | |
| CALM1)- | −0.1230607 | 0.07240028 | 0.05648191 | 0.00039526 | −0.1256797 | −0.0426877 | −0.0676179 | −0.1542819 | 0.20092227 |
| SCTR | | | | | | | | | |
| RTN4)- | −0.1406734 | −0.1608696 | −0.039217 | −0.1579762 | −0.0489734 | −0.2237011 | 0.03358271 | −0.0048101 | 0.27550315 |
| RTN4R | | | | | | | | | |
| TNFSF13B)- | −0.0010876 | 0.04988797 | 0.13979881 | 0.01699661 | 0.1820551 | 0.04908744 | 0.12016494 | −0.0523836 | 0.01409843 |
| TNFRSF17 | | | | | | | | | |
| ST6GAL1)- | 0.17790017 | 0.06711279 | −0.0444862 | −0.0306983 | 0.19000232 | 0.02141751 | −0.2696148 | 0.03354312 | −0.1785244 |
| CD22 | | | | | | | | | |
| CALM2)- | −0.0054038 | 0.43314997 | −0.3428685 | 0.45778846 | −0.2065648 | −0.0378266 | −0.0765885 | 0.0093578 | −0.3182582 |
| KCNQ1 | | | | | | | | | |
| CALML3)- | −0.1801662 | −0.0237373 | 0.02332025 | 0.16103314 | −0.2667853 | 0.03374638 | −0.0930662 | 0.04494382 | 0.30244738 |
| KCNQ1 | | | | | | | | | |
| CALM1)- | 0.03650982 | 0.16733645 | 0.0918169 | 0.01119931 | −0.1392697 | 0.16949489 | −0.0489718 | −0.1563808 | 0.06225502 |
| KCNQ1 | | | | | | | | | |
| CALM3)- | −0.0904178 | −0.080506 | 0.00197739 | −0.1247735 | 0.05721066 | 0.13417247 | −0.1991827 | −0.1444529 | 0.10066208 |
| KCNQ1 | | | | | | | | | |
| FASLG)- | −0.1151488 | 0.26259393 | −0.1225053 | −0.0961792 | −0.106179 | 0.01409796 | 0.14256057 | −0.1625218 | −0.169697 |
| TNFRSF1A | | | | | | | | | |
| LTA)- | −0.1259557 | 0.01225902 | 0.42057369 | −0.0156157 | 0.03961897 | 0.00039535 | −0.036843 | −0.0137027 | 0.19341238 |
| TNFRSF1A | | | | | | | | | |
| TNF)- | −0.1377287 | −0.036788 | 0.17499424 | 0.12606677 | −0.1563592 | −0.2770529 | −0.0950404 | 0.00935472 | −0.2088274 |
| TNFRSF1A | | | | | | | | | |
| LTB)- | −0.1236278 | 0.18533665 | −0.1909694 | 0.22840767 | 0.13222161 | −0.0549461 | 0.01529082 | −0.0899898 | 0.17444005 |
| TNFRSF1A | | | | | | | | | |
| IL2)-NGFR | −0.0906303 | −0.0992647 | 0.01032323 | 0.00500692 | 0.30258607 | −0.054948 | −0.0140989 | −0.0691086 | 0.40474308 |
| RTN4)- | 0.04769748 | −0.057971 | 0.00382119 | 0.10369248 | 0.03017525 | 0.05968969 | −0.1116419 | −0.1372995 | 0.14097961 |
| NGFR | | | | | | | | | |
| NTF4)- | −0.0262759 | 0.09204197 | 0.01770466 | 0.07866131 | 0.09336188 | 0.19577678 | −0.0262915 | 0.16031364 | 0.27303575 |
| NGFR | | | | | | | | | |
| APP)- | −0.0064894 | −0.1724694 | −0.217947 | 0.05388669 | −0.1429654 | 0.0452599 | −0.0893369 | 0.11463206 | 0.00368906 |
| NGFR | | | | | | | | | |
| BDNF)- | −0.1512226 | −0.1533544 | 0.28166091 | 0.12446465 | −0.4083556 | 0.23761202 | 0.23249992 | −0.1290599 | 0.24479578 |
| NGFR | | | | | | | | | |
| BDNF)- | 0.01845262 | −0.0842341 | −0.0370505 | 0.20105437 | −0.0087994 | −0.1438361 | −0.1445188 | 0.19947299 | 0.1068652 |
| DRD4 | | | | | | | | | |
| NPY)- FAP | 0.41577702 | −0.2025834 | −0.375033 | −0.1384149 | −0.2298873 | 0.2961987 | −0.1952826 | −0.042029 | 0.02529644 |
| CD99)- | −0.0084321 | 0.04571805 | −0.1997825 | −0.2216147 | 0.1016089 | −0.0662077 | −0.217453 | 0.04782609 | 0.09025033 |
| PILRA | | | | | | | | | |
| LAMA3)- | 0.3687747 | −0.3911726 | 0.45652174 | −0.4682477 | 0.39604743 | 0.36166008 | −0.4079051 | −0.3417655 | 0.62516469 |
| ITGB4 | | | | | | | | | |
| LAMA2)- | 0.23091113 | −0.0739838 | 0.24909414 | 0.19736495 | −0.1961792 | −0.202108 | 0.38959157 | 0.41554677 | −0.3235837 |
| ITGB4 | | | | | | | | | |
| LAMC3)- | −0.064446 | −0.114988 | 0.04915818 | −0.1207629 | 0.09915341 | 0.14046184 | 0.05586298 | 0.01475626 | −0.4194993 |
| ITGB4 | | | | | | | | | |
| LAMB2)- | 0.19591568 | −0.0513834 | 0.21673254 | 0.28909313 | −0.3983925 | −0.3987219 | −0.0490119 | −0.0325428 | 0.19657444 |
| ITGB4 | | | | | | | | | |
| LAMA5)- | 0.52597253 | −0.2602194 | 0.51951645 | −0.2013967 | 0.36702023 | 0.37426708 | −0.1975097 | −0.2127281 | 0.24494367 |
| ITGB4 | | | | | | | | | |
| LAMC1)- | 0.2458822 | −0.115562 | 0.15654237 | 0.20712145 | −0.3428967 | −0.3511973 | 0.27325428 | 0.35968379 | −0.0969697 |
| ITGB4 | | | | | | | | | |
| LAMA1)- | 0.15748551 | −0.0478387 | 0.08118081 | −0.1234763 | −0.0614087 | −0.1340186 | 0.03267457 | −0.0293808 | −0.0671937 |
| ITGB4 | | | | | | | | | |
| LAMB3)- | 0.60355731 | −0.3338603 | 0.61449275 | −0.2508564 | 0.57246377 | 0.63662714 | −0.3077734 | −0.3218709 | 0.27444005 |
| ITGB4 | | | | | | | | | |
| LAMB1)- | 0.21936759 | 0.07944664 | 0.22806324 | 0.10178201 | −0.0922297 | −0.0382753 | 0.10263505 | 0.15046113 | 0.16284585 |
| ITGB4 | | | | | | | | | |
| LAMC2)- | 0.50974967 | −0.2868248 | 0.51501976 | −0.3300395 | 0.68023715 | 0.73333333 | −0.3280632 | −0.3802372 | 0.50079051 |
| ITGB4 | | | | | | | | | |
| TGFB1)- | 0.16291025 | −0.1263304 | 0.23241629 | −0.0489476 | −0.1767695 | −0.0096515 | −0.1993208 | 0.09637999 | −0.2213439 |
| TGFBR3 | | | | | | | | | |
| TGFB3)- | 0.03505073 | −0.0899298 | 0.07163593 | 0.05843407 | −0.028725 | −0.0832729 | 0.09293283 | 0.08952864 | −0.2254282 |
| TGFBR3 | | | | | | | | | |
| INHA)- | 0.18750413 | −0.1416285 | 0.20798423 | 0.07464014 | −0.1368314 | 0.06005007 | −0.3064114 | 0.16055076 | 0.19552686 |
| TGFBR3 | | | | | | | | | |
| TGFB2)- | −0.0235883 | −0.006391 | 0.00224289 | 0.05415733 | −0.2566709 | −0.369692 | −0.2007125 | 0.139469 | −0.0661418 |
| TGFBR3 | | | | | | | | | |
| INHBA)- | 0.07911987 | −0.0992095 | 0.13837691 | −0.1366975 | 0.05487769 | −0.2351934 | 0.17498282 | 0.13017557 | −0.0939394 |
| TGFBR3 | | | | | | | | | |
| IL2)- IL2RB | 0.31085338 | 0.22629704 | −0.2708781 | −0.1287305 | −0.0064648 | 0.0084327 | 0.14848298 | 0.00342556 | −0.0090909 |
| KNG1)- | 0.00961919 | 0.26847185 | 0.06425888 | −0.149412 | 0.02754712 | 0.06798867 | 0.0002636 | 0.13788333 | −0.0990777 |
| BDKRB1 | | | | | | | | | |
| PROC)- | 0.02254747 | −0.1298744 | 0.18451694 | 0.00929007 | −0.0896984 | −0.1612967 | −0.0686223 | 0.14967555 | 0.00895916 |
| PROCR | | | | | | | | | |
| PF4)- | −0.1062806 | −0.019659 | −0.0480576 | 0.24711773 | −0.2883513 | 0.14849952 | −0.0754804 | 0.22531129 | 0.17016371 |
| PROCR | | | | | | | | | |
| TNFSF10)- | −0.0432821 | −0.0383412 | 0.29491237 | 0.02213439 | 0.07374214 | 0.14795784 | 0.00425069 | −0.1734576 | 0.07490365 |
| TNFRSF10A | | | | | | | | | |
| APP)- | 0.00658805 | −0.0308321 | −0.0495487 | 0.21087651 | −0.0291221 | 0.07760466 | −0.1118102 | −0.0583682 | −0.174314 |
| APLP1 | | | | | | | | | |
| CXCL8)- | −0.0254307 | 0.03438962 | −0.0159489 | 0.09051383 | 0.11005306 | −0.156461 | 0.17845729 | −0.1300438 | −0.244137 |
| CD79A | | | | | | | | | |
| FN1)- | −0.1122567 | 0.28300395 | −0.2292662 | −0.2471673 | 0.23796502 | −0.0597516 | −0.2377673 | −0.0992786 | 0.2400527 |
| CD79A | | | | | | | | | |
| KITLG)- | −0.1679953 | −0.0849858 | −0.1234141 | 0.03142396 | −0.12977 | 0.11522119 | 0.00579978 | −0.0099476 | 0.04519253 |
| EPOR | | | | | | | | | |
| LAMA1)- | 0.10747233 | −0.1711697 | 0.18220158 | 0.03923959 | −0.2651468 | −0.2177637 | −0.2292566 | −0.0453228 | −0.0574478 |
| ITGB8 | | | | | | | | | |
| VTN)- | 0.07931697 | −0.0937624 | 0.13183666 | 0.06661615 | 0.04954866 | 0.06391039 | 0.33617708 | 0.33214756 | −0.2367086 |
| ITGB8 | | | | | | | | | |
| FN1)- | 0.19736495 | −0.0611371 | 0.26245924 | 0.39205481 | −0.367601 | −0.298946 | 0.3078494 | 0.25968379 | −0.0073127 |
| ITGB8 | | | | | | | | | |
| APP)- | 0.07365682 | 0.11351209 | 0.10519726 | 0.02213512 | 0.11198262 | −0.1898017 | −0.0758634 | 0.14816523 | −0.267466 |
| TSPAN12 | | | | | | | | | |
| ADAM10)- | 0.07392278 | 0.22729519 | −0.0684859 | 0.23651097 | −0.0827868 | 0.23025991 | 0.21809916 | −0.0855788 | −0.4906617 |
| TSPAN12 | | | | | | | | | |
| C1QB)- | 0.17995519 | 0.07359821 | 0.18759884 | −0.3173913 | 0.09368206 | 0.08353647 | 0.0719415 | 0.05072798 | −0.3150198 |
| C1QBP | | | | | | | | | |
| GNRH1)- | −0.0553029 | −0.0371713 | 0.13392239 | −0.2246377 | −0.177624 | −0.1301884 | 0.02563849 | 0.09250231 | 0.10527009 |
| GNRHR | | | | | | | | | |
| HBEGF)- | 0.14407589 | −0.0146909 | 0.25205046 | −0.0673254 | 0.25586298 | 0.19222661 | 0.26785244 | 0.36152833 | −0.3924901 |
| CD82 | | | | | | | | | |
| IL10)- | −0.1192594 | −0.2446465 | 0.38047527 | 0.0628562 | 0.03750577 | 0.09382309 | 0.00995156 | −0.0221344 | 0.11054018 |
| IL10RA | | | | | | | | | |
| S100A8)- | −0.2329797 | −0.2294131 | 0.09110694 | 0.00724638 | −0.0134476 | 0.00573141 | 0.0704374 | −0.0795178 | −0.0210152 |
| CD69 | | | | | | | | | |
| TNFSF14)- | −0.2530086 | 0.02848572 | −0.3260907 | −0.3553477 | 0.30467438 | 0.17582924 | 0.29479198 | 0.17820086 | −0.1343918 |
| LTBR | | | | | | | | | |
| LTA)- | 0.08001319 | −0.3015983 | 0.31206541 | −0.1981946 | −0.074262 | 0.06760229 | 0.1175308 | 0.19328063 | 0.42358366 |
| LTBR | | | | | | | | | |
| LTB)- | −0.0299334 | 0.01265907 | −0.143314 | 0.01818302 | 0.28617077 | 0.15429211 | 0.17313394 | −0.0355731 | 0.25665349 |
| LTBR | | | | | | | | | |
| CLEC2D)- | 0.01900868 | 0.16401588 | 0.1619552 | 0.2164833 | −0.2936116 | 0.22379603 | −0.3035138 | 0.36034256 | 0.40711462 |
| KLRB1 | | | | | | | | | |
| IL33)- | −0.3928678 | 0.03077836 | 0.4364907 | 0.15620779 | −0.2166178 | 0.29447812 | −0.2012782 | 0.3173777 | 0.32028986 |
| ILIRL1 | | | | | | | | | |
| GNAI2)- | 0.08646084 | 0.24497596 | −0.0734266 | −0.084585 | −0.0590991 | 0.02477351 | 0.1712949 | −0.1180036 | 0.03175231 |
| OPRD1 | | | | | | | | | |
| POMC)- | −0.0243886 | 0.0782285 | 0.18426876 | 0.11220927 | 0.19507862 | −0.0168045 | 0.11213359 | 0.20906635 | 0.21753022 |
| OPRD1 | | | | | | | | | |
| CXCL13)- | −0.1849703 | −0.1182403 | 0.17616397 | 0.26278863 | −0.2449789 | 0.31873888 | −0.0188642 | 0.35664636 | 0.26205534 |
| OPRD1 | | | | | | | | | |
| PENK)- | 0.03725316 | 0.0187224 | 0.30524757 | −0.02207 | 0.14294205 | 0.18054229 | 0.20209765 | −0.0612749 | 0.29209486 |
| OPRD1 | | | | | | | | | |
| CALM2)- | −0.023455 | −0.1277997 | 0.15231506 | −0.1561265 | 0.15725822 | −0.0092239 | 0.03657934 | −0.2207142 | −0.1932806 |
| SELL | | | | | | | | | |
| PODXL2)- | 0.15947282 | 0.2574949 | 0.05270444 | −0.1880826 | −0.2213288 | 0.03788503 | 0.12226068 | 0.10225326 | −0.1606061 |
| SELL | | | | | | | | | |
| VCAN)- | −0.2866744 | 0.04769591 | 0.09662537 | −0.1790514 | −0.046927 | 0.13796284 | 0.11487891 | −0.2810647 | −0.0656126 |
| SELL | | | | | | | | | |
| CD34)- | 0.14765013 | 0.13688791 | −0.1325377 | 0.01679897 | 0.14717903 | 0.10765936 | 0.05852695 | 0.13480037 | −0.0836627 |
| SELL | | | | | | | | | |
| MUC7)- | −0.1204494 | −0.046579 | 0.07748336 | −0.1511199 | −0.1262152 | −0.0336013 | 0.09211048 | 0.22678307 | 0.30699298 |
| SELL | | | | | | | | | |
| CALM1)- | −0.2799539 | −0.0401199 | 0.22785397 | −0.0534914 | −0.0075795 | 0.12794835 | 0.07441095 | −0.1626038 | −0.1785244 |
| SELL | | | | | | | | | |
| PODXL)- | −0.0023721 | −0.049412 | 0.25126889 | −0.1785303 | 0.23474165 | −0.2183495 | 0.01779594 | −0.019239 | −0.0602787 |
| SELL | | | | | | | | | |
| CALM3)- | 0.28074452 | 0.01541553 | −0.1530451 | 0.04545455 | −0.2681826 | 0.08103835 | 0.2417532 | −0.1436289 | −0.2341238 |
| SELL | | | | | | | | | |
| CFH)- | −0.2494482 | −0.1474357 | 0.26970738 | −0.0221344 | −0.3000165 | 0.2706549 | 0.25117815 | −0.1185927 | −0.0876153 |
| SELL | | | | | | | | | |
| HLA-E)- | −0.6178883 | −0.5289409 | 0.6791682 | −0.2125165 | 0.35132466 | −0.184058 | 0.1942138 | −0.0758893 | −0.1656126 |
| KLRK1 | | | | | | | | | |
| MICB)- | −0.1483629 | −0.0404506 | 0.18954723 | 0.017326 | 0.06412496 | 0.01943411 | 0.27118758 | −0.1790514 | 0.02147563 |
| KLRK1 | | | | | | | | | |
| MICA)- | −0.050547 | 0.14814816 | 0.0494462 | −0.0329392 | 0.05779814 | −0.1027043 | 0.18096746 | −0.1412385 | −0.0909091 |
| KLRK1 | | | | | | | | | |
| RAET1L)- | −0.3648943 | −0.1964815 | 0.33616769 | 0.20608776 | −0.2409043 | 0.2515483 | 0.01245634 | 0.09901838 | 0.11680611 |
| KLRK1 | | | | | | | | | |
| ULBP2)- | −0.004943 | 0.07961511 | −0.1363816 | 0.17214006 | −0.227436 | 0.26048289 | −0.202847 | 0.21857708 | 0.23056653 |
| KLRK1 | | | | | | | | | |
| RAET1E)- | 0.00744621 | −0.0973279 | −0.0606151 | −0.0631794 | 0.04333356 | −0.0422953 | −0.0618163 | 0.07193676 | 0.14057971 |
| KLRK1 | | | | | | | | | |
| RAETIG)- | −0.1937313 | 0.02109357 | 0.14388869 | 0.1015185 | −0.2451643 | 0.23650318 | 0.14683012 | −0.0598155 | 0.0571805 |
| KLRK1 | | | | | | | | | |
| BMP7)- | −0.2397337 | −0.1438882 | 0.309206 | 0.30297441 | −0.09155 | 0.16707293 | 0.02793609 | 0.41899993 | 0.11502355 |
| ACVR2A | | | | | | | | | |
| LEFTY1)- | 0.12275558 | −0.1298676 | −0.1860297 | −6.59E−05 | 0.10963597 | −0.2881613 | 0.12346818 | 0.09157087 | 0.22147563 |
| ACVR2A | | | | | | | | | |
| INHA)- | −0.0991894 | −0.0473852 | −0.038691 | 0.36305544 | 0.14304069 | −0.1237236 | −0.1650469 | 0.19171223 | −0.0329392 |
| ACVR2A | | | | | | | | | |
| INHBA)- | −0.0857077 | −0.0038208 | 0.02839636 | 0.11054383 | −0.1626091 | −0.0316556 | 0.06746607 | −0.0576435 | −0.0488801 |
| ACVR2A | | | | | | | | | |
| INHBB)- | 0.22728021 | 0.00606061 | −0.0975754 | −0.0354414 | −0.0917776 | 0.22326164 | −0.0842667 | −0.116934 | 0.13043478 |
| ACVR2A | | | | | | | | | |
| TDGF1)- | 0.19010569 | −0.0763828 | −0.1888711 | −0.0808327 | 0.10074123 | −0.2401344 | −0.0278034 | 0.0565236 | 0.26758893 |
| ACVR2A | | | | | | | | | |
| BMP2)- | 0.28415193 | −0.1053429 | −0.0538644 | 0.10889687 | −0.0272113 | 0.14668292 | 0.08355298 | −0.0664141 | −0.1001449 |
| ACVR2A | | | | | | | | | |
| BMP6)- | −0.0074484 | −0.0092278 | −0.109529 | −0.149888 | −0.0011202 | 0.077549 | −0.1063416 | 0.19810264 | −0.1195692 |
| ACVR2A | | | | | | | | | |
| GDF11)- | 0.16079078 | 0.05864716 | 0.15668765 | −0.1730681 | 0.02698162 | −0.2561847 | −0.1474503 | 0.07964689 | −0.3155468 |
| ACVR2A | | | | | | | | | |
| FLT3LG)- | 0.01608968 | −0.1294258 | 0.11435452 | 0.15494071 | −0.0519378 | −0.2473317 | −0.1174532 | 0.15614706 | 0.11198946 |
| FLT3 | | | | | | | | | |
| CD55)- | 0.00184453 | −0.4285903 | 0.26005601 | −0.4795784 | 0.29042992 | −0.0105402 | −0.201746 | −0.2689065 | 0.24176548 |
| ADGRE5 | | | | | | | | | |
| CALM2)- | −0.1268199 | 0.15915679 | 0.03790877 | 0.18722003 | 0.0997495 | −0.1506028 | −0.139768 | −0.1207589 | −0.4314888 |
| PDE1B | | | | | | | | | |
| CALM1)- | −0.2567447 | 0.07345433 | 0.36393615 | −0.1433465 | 0.00969146 | −0.0460505 | 0.25065936 | −0.0526385 | 0.17944664 |
| PDE1B | | | | | | | | | |
| CALM3)- | 0.40656191 | 0.12530057 | −0.1823637 | −0.054809 | −0.2236288 | 0.28104618 | 0.04647944 | −0.2399368 | 0.07364954 |
| PDE1B | | | | | | | | | |
| APOA2)- | −0.0952161 | 0.01713175 | 0.03993016 | −0.2708827 | −0.4234519 | 0.15580223 | −0.1363022 | 0.05059622 | 0.04657597 |
| LRP1 | | | | | | | | | |
| AGRN)- | 0.02654984 | 0.4060081 | 0.32240851 | 0.3002635 | 0.18234519 | 0.11620936 | −0.1607378 | −0.2823545 | −0.2114625 |
| LRP1 | | | | | | | | | |
| GPC3)- | −0.1317776 | 0.07488466 | 0.00685564 | 0.12898551 | 0.12134387 | 0.06890873 | −0.2255599 | 0.08274317 | −0.1884058 |
| LRP1 | | | | | | | | | |
| PDGFB)- | −0.1353927 | 0.2583815 | 0.25053577 | −0.2819592 | −0.2314305 | 0.15521444 | −0.1852437 | −0.0664712 | −0.0274045 |
| LRP1 | | | | | | | | | |
| A2M)- | 0.26041701 | 0.10039526 | 0.17009223 | 0.44784084 | 0.44052834 | −0.1864089 | 0.28036891 | −0.2639744 | 0.39762846 |
| LRP1 | | | | | | | | | |
| APOC2)- | 0.03544487 | −0.0972396 | 0.01343962 | −0.0492436 | −0.0831273 | 0.0806909 | −0.2101449 | 0.15929378 | −0.2262187 |
| LRP1 | | | | | | | | | |
| SERPINA1)- | −0.095853 | −0.0185771 | −0.1110672 | 0.17272727 | 0.0226614 | −0.1361046 | 0.24571805 | −0.0819526 | 0.17167325 |
| LRP1 | | | | | | | | | |
| SERPINE2)- | −0.2793425 | 0.01297846 | −0.065024 | −0.0982894 | −0.1683642 | 0.09321645 | 0.09407115 | 0.06153035 | 0.07206851 |
| LRP1 | | | | | | | | | |
| HSPG2)- | −0.171613 | 0.36785244 | 0.51383399 | −0.1762846 | −0.0525692 | 0.12780395 | 0.39341238 | −0.1709542 | 0.16192358 |
| LRP1 | | | | | | | | | |
| MMP13)- | −0.3224956 | −0.0080374 | 0.09249621 | −0.1156823 | −0.0603445 | 0.07556493 | 0.00961792 | 0.04525841 | 0.05836627 |
| LRP1 | | | | | | | | | |
| APOE)- | 0.21952103 | 0.2347322 | 0.31787338 | −0.1184453 | −0.3007905 | −0.2623934 | −0.2449275 | −0.2885471 | −0.0472991 |
| LRP1 | | | | | | | | | |
| PCSK9)- | −0.1089363 | −0.0463936 | 0.09588455 | −0.1199064 | −0.1325559 | 0.20832784 | 0.23833992 | −0.3249119 | 0.16864295 |
| LRP1 | | | | | | | | | |
| MMP9)- | −0.1162982 | −0.0610793 | −0.1170851 | 0.12920442 | 0.18309999 | 0.11365883 | 0.19749671 | −0.1675945 | 0.26429513 |
| LRP1 | | | | | | | | | |
| HSP90B1)- | 0.01416431 | −0.0804374 | 0.00283277 | −0.2554677 | −0.1727273 | 0.1258276 | 0.4115942 | 0.01805066 | 0.38761528 |
| LRP1 | | | | | | | | | |
| MDK)- | −0.0988175 | −0.0459816 | −0.0754941 | −0.35639 | −0.3516469 | 0.304819 | 0.18709444 | 0.1818631 | 0.07332257 |
| LRP1 | | | | | | | | | |
| PLAU)- | −0.116368 | 0.34823577 | 0.38118144 | −0.3548206 | −0.3252413 | 0.02931682 | 0.08300395 | −0.1692414 | 0.1400527 |
| LRP1 | | | | | | | | | |
| LRPAP1)- | −0.1664305 | 0.25080726 | 0.35710051 | −0.1766857 | −0.1585032 | 0.01647012 | −0.2758984 | 0.00401871 | −0.1585691 |
| LRP1 | | | | | | | | | |
| C3)- LRP1 | 0.13848536 | 0.32920482 | 0.34383029 | −0.2314888 | −0.2508564 | 0.12609111 | −0.0285903 | 0.20824138 | 0.08036891 |
| THBS1)- | −0.083929 | 0.38076416 | 0.41554677 | −0.3602108 | −0.3115942 | 0.25165519 | 0.27536232 | −0.0761553 | 0.27391304 |
| LRP1 | | | | | | | | | |
| HSP90AA1)- | −0.3123291 | 0.17061924 | 0.08274045 | 0.14229249 | 0.01357049 | −0.2724069 | 0.34268775 | 0.25804539 | 0.2171278 |
| LRP1 | | | | | | | | | |
| APOA4)- | 0.17263533 | −0.0936944 | 0.01904197 | 0.06785691 | −0.1397325 | 0.00704944 | −0.0007905 | −0.4003426 | 0.2171278 |
| LRP1 | | | | | | | | | |
| CALR)- | −0.1199723 | 0.23875091 | 0.1570591 | 0.02213439 | −0.1137022 | 0.07734115 | −0.1018445 | 0.01133107 | 0.11027668 |
| LRP1 | | | | | | | | | |
| C1QB)- | 0.34026291 | −0.0560058 | 0.05257956 | 0.08603426 | −0.1263505 | −0.4966567 | −0.1768116 | −0.503508 | 0.07681159 |
| LRP1 | | | | | | | | | |
| C4BPA)- | −0.2578051 | −0.1609416 | −0.1870509 | −0.406601 | −0.4551533 | 0.34666315 | −0.1596891 | 0.06548521 | −0.1588985 |
| LRP1 | | | | | | | | | |
| VWF)- | −0.0338637 | 0.09078332 | 0.06884512 | 0.35020097 | 0.40614088 | −0.1677594 | −0.2111989 | 0.227939 | −0.2183136 |
| LRP1 | | | | | | | | | |
| CCN2)- | −0.2980335 | 0.53109354 | 0.58142292 | −0.3322793 | −0.4370224 | 0.15415528 | 0.49841897 | −0.3400639 | 0.53359684 |
| LRP1 | | | | | | | | | |
| HPX)- | −0.1823574 | 0.2957691 | 0.14076711 | −0.1048921 | −0.1968704 | −0.1094419 | −0.1135705 | −0.1944069 | 0.00974967 |
| LRP1 | | | | | | | | | |
| APP)- LRP1 | −0.058963 | 0.29197273 | 0.18274647 | −0.1888011 | −0.0641634 | 0.22319576 | 0.23241107 | −0.0919003 | 0.25810277 |
| LPL)- LRP1 | 0.15385658 | −0.0156426 | 0.09913555 | −0.0603445 | −0.1478968 | −0.0409118 | 0.16866517 | −0.1869214 | 0.29898538 |
| LIPC)- | −0.3353436 | −0.1136229 | −0.1098662 | −0.2715594 | −0.3701166 | 0.04374609 | 0.02371542 | −0.0751013 | 0.0914361 |
| LRP1 | | | | | | | | | |
| PF4)- LRP1 | −0.1664469 | 0.18537473 | 0.09512824 | 0.00652217 | 0.10712168 | 0.26965774 | −0.2933562 | −0.0258252 | −0.228993 |
| IL9)- IL9R | 0.16364595 | −0.0293236 | 0.01206408 | −0.1606643 | 0.14438291 | −0.0656472 | −0.0767786 | 0.08084335 | 0.22674572 |
| APP)- | −0.2306552 | −0.0432162 | 0.12964854 | −0.1403162 | 0.14202899 | −0.2013967 | −0.0631094 | −0.001647 | −0.0396574 |
| PTGER2 | | | | | | | | | |
| F12)- CD93 | 0.19168354 | −0.2096374 | −0.0576619 | 0.16118617 | −0.215694 | −0.1018781 | −0.0063944 | 0.07470602 | 0.09875161 |
| COL4A3)- | −0.0255142 | 0.03171151 | −0.1953754 | 0.09745009 | 0.21975344 | −0.2321935 | −0.3299276 | 0.24321476 | 0.22898551 |
| CD93 | | | | | | | | | |
| COL4A5)- | 0.13271829 | −0.2056013 | 0.11859546 | 0.13847167 | 0.18510222 | −0.3011858 | 0.22205746 | −0.0250996 | −0.1496097 |
| CD93 | | | | | | | | | |
| KNG1)- | 0.03215074 | 0.06206147 | 0.08975839 | −0.0558648 | −0.2125977 | −0.0390658 | 0.10804221 | 0.14321476 | −0.0013175 |
| CD93 | | | | | | | | | |
| SFTPA2)- | −0.0176583 | −0.1739474 | 0.03801016 | −0.0493494 | 0.03378936 | −0.0600889 | −0.0669743 | 0.37681159 | 0.24479578 |
| CD93 | | | | | | | | | |
| COL4A2)- | 0.00474308 | 0.00632411 | 0.10085698 | −0.0602108 | −0.2112723 | 0.22990777 | 0.21951224 | −0.0532279 | −0.0610013 |
| CD93 | | | | | | | | | |
| COL4A4)- | 0.01925869 | 0.2469333 | 0.04207366 | 0.1141765 | 0.14106666 | −0.1412703 | 0.03474077 | −0.0724003 | −0.0951283 |
| CD93 | | | | | | | | | |
| MBL2)- | −0.258715 | −0.2339374 | 0.28519619 | 0.04539016 | −0.1352055 | 0.15033433 | −0.0075813 | −0.1200343 | 0.14375124 |
| CD93 | | | | | | | | | |
| COL1A2)- | 0.36508564 | −0.055863 | −0.1747528 | −0.1818182 | 0.29887943 | −0.1142292 | −0.0609756 | 0.32226614 | −0.0852437 |
| CD93 | | | | | | | | | |
| C1QA)- | 0.07898811 | −0.044929 | 0.12155973 | 0.11119895 | −0.0074489 | −0.0530962 | −0.0427159 | −0.116469 | 0.08129117 |
| CD93 | | | | | | | | | |
| CCL21)- | 0.09515965 | −0.3173087 | −0.1212221 | 0.19704206 | 0.10813483 | 0.20481571 | 0.18503509 | 0.06581244 | 0.13597286 |
| CCR7 | | | | | | | | | |
| CCL19)- | 0.29669896 | 0.04151018 | 0.0795492 | 0.0668687 | 0.40711698 | −0.1734633 | 0.20301793 | −0.099473 | 0.31040843 |
| CCR7 | | | | | | | | | |
| HLA-B)- | 0.23149643 | 0.05263678 | 0.13133276 | −0.059552 | 0.10039856 | 0.2229249 | 0.29493725 | 0.28932806 | −0.3644269 |
| CANX | | | | | | | | | |
| TNF)- | −0.2583815 | −0.0023734 | 0.17749332 | 0.46380444 | −0.3417096 | −0.1541453 | −0.0272293 | 0.31094568 | −0.0831357 |
| TRAF2 | | | | | | | | | |
| FADD)- | −0.07809 | 0.00929926 | 0.30399342 | 0.17694335 | −0.0052744 | −0.067657 | −0.1714691 | 0.08782448 | −0.1483678 |
| TRAF2 | | | | | | | | | |
| GSTP1)- | −0.075233 | 0.13794466 | 0.03962421 | 0.17588933 | −0.0523488 | 0.05191212 | 0.1616615 | 0.11779044 | −0.3397892 |
| TRAF2 | | | | | | | | | |
| F2)- F2RL3 | −0.0378888 | 0.00777518 | 0.39868809 | 0.01436195 | −0.203711 | 0.10310637 | 0.28527176 | 0.1766256 | 0.50541849 |
| GAL)- | −0.3209047 | −0.0699522 | 0.23365875 | −0.0447958 | 0.01436715 | 0.19263456 | 0.2618381 | 0.02114764 | −0.0057971 |
| GALR3 | | | | | | | | | |
| S100A9)- | −0.307966 | −0.4185939 | 0.37984318 | 0.24097497 | −0.1731339 | 0.19617918 | −0.2350616 | 0.17338603 | 0.23017128 |
| CD68 | | | | | | | | | |
| S100A8)- | −0.1370811 | −0.1049198 | 0.1311254 | 0.06996047 | −0.0861058 | −0.0285903 | −0.1221465 | −0.0258902 | 0.03583781 |
| CD68 | | | | | | | | | |
| PDGFB)- | 0.0241659 | 0.01035177 | −0.3012203 | 0.0053365 | 0.05727657 | −0.1571353 | 0.19383756 | 0.06654149 | 0.03030503 |
| ART1 | | | | | | | | | |
| AFDN)- | −0.1594465 | 0.08649824 | −0.2315928 | −0.0213439 | −0.0878904 | −0.0005271 | 0.16102784 | 0.24345248 | −0.1615995 |
| NECTIN2 | | | | | | | | | |
| ADCYAP1)- | −0.1341371 | −0.1932997 | 0.17097763 | 0.28421781 | −0.0051732 | 0.04460256 | −0.2439378 | 0.25520422 | 0.11528327 |
| RAMP2 | | | | | | | | | |
| GHRH)- | −0.1532681 | −0.2332312 | −0.1319442 | 0.19980895 | −0.0788442 | −0.0002635 | −0.2319452 | 0.13860343 | 0.32859025 |
| RAMP2 | | | | | | | | | |
| CALCA)- | 0.0665744 | −0.0866785 | 0.20409443 | 0.25885533 | −0.2434 | 0.1907147 | −0.1676331 | 0.18550725 | 0.52358366 |
| RAMP2 | | | | | | | | | |
| ADM)- | −0.1144533 | −0.0405891 | 0.16243203 | 0.30759602 | 0.06009885 | −0.1562685 | −0.0444466 | 0.01719424 | 0.28004875 |
| RAMP2 | | | | | | | | | |
| GCG)- | −0.1465327 | 0.00500741 | 0.22503048 | 0.05455264 | −0.1106498 | 0.22308605 | −0.001252 | 0.17826087 | 0.30118577 |
| RAMP2 | | | | | | | | | |
| TSHB)- | 0.13238361 | −0.0682036 | −0.0227625 | 0.11252388 | −0.0312026 | 0.2320311 | 0.03617554 | 0.37575758 | 0.45256917 |
| RAMP2 | | | | | | | | | |
| PTH)- | −0.1298128 | −0.1210443 | 0.44378139 | −0.0120561 | −0.0914003 | −0.0084327 | 0.15722193 | 0.06218709 | −0.0437418 |
| RAMP2 | | | | | | | | | |
| ADM2)- | −0.2407511 | 0.00362379 | 0.25567602 | 0.26482213 | −0.1089879 | 0.20447958 | −0.2277355 | 0.26133931 | 0.27444909 |
| RAMP2 | | | | | | | | | |
| PTHLH)- | 0.09836501 | −0.0429193 | −0.2771367 | 0.39251598 | 0.18490939 | −0.0179854 | 0.2144834 | 0.0773386 | 0.44057971 |
| RAMP2 | | | | | | | | | |
| NPS)- | −0.2440511 | −0.0799421 | 0.36227733 | 0.12305665 | −0.2008434 | −0.0985507 | −0.1931998 | 0.1911726 | 0.18326746 |
| RAMP2 | | | | | | | | | |
| FSHB)- | −0.0088956 | 0.09804956 | 0.15795544 | 0.09046286 | −0.1003065 | −0.3455115 | −0.184436 | 0.09710145 | 0.4085639 |
| RAMP2 | | | | | | | | | |
| POMC)- | −0.2865522 | 0.14090356 | −0.0124592 | −0.0105423 | −0.0935873 | 0.20801213 | 0.06431419 | 0.02852531 | 0.26094404 |
| RAMP2 | | | | | | | | | |
| CALCB)- | −0.0044146 | −0.0264216 | 0.00233968 | 0.23641815 | −0.0080079 | −0.0289263 | −0.0185161 | −0.0463768 | 0.23069829 |
| RAMP2 | | | | | | | | | |
| GIP)- | −0.1309132 | 0.00059296 | 0.03650982 | 0.16424548 | 0.23565851 | −0.1294594 | −0.1142631 | 0.08742053 | 0.30956224 |
| RAMP2 | | | | | | | | | |
| CRH)- | −0.0102132 | −0.0214806 | 0.00402043 | 0.30290853 | −0.0566044 | 0.06113508 | −0.1377833 | 0.18208169 | 0.26653491 |
| RAMP2 | | | | | | | | | |
| INSL3)- | −0.1763659 | −0.1512555 | 0.08606368 | 0.11844532 | −0.1734976 | 0.22542819 | −0.1153137 | 0.0687747 | 0.21805007 |
| RAMP2 | | | | | | | | | |
| CALCA)- | −0.0033288 | −0.0667062 | 0.04098388 | 0.10188145 | −0.0167787 | 0.06036642 | 0.01443306 | 0.06133272 | 0.18418972 |
| RAMP1 | | | | | | | | | |
| ADM)- | −0.2996178 | 0.10489902 | 0.04238629 | −0.011595 | −0.1954194 | 0.51823303 | −0.2004877 | −0.0270769 | 0.2170032 |
| RAMP1 | | | | | | | | | |
| ADM2)- | −0.0054029 | 0.23857684 | 0.36942194 | 0.14545455 | −0.0259663 | 0.06607596 | −0.1140842 | 0.25205877 | 0.22200995 |
| RAMP1 | | | | | | | | | |
| CALCB)- | −0.0119263 | −0.1437702 | 0.29488152 | 0.2101934 | −0.031147 | −0.1440432 | −0.0705177 | 0.16021608 | 0.10237154 |
| RAMP1 | | | | | | | | | |
| SST)- | −0.1092587 | −0.0537691 | 0.19040401 | 0.09822781 | 0.22845282 | −0.0484254 | 0.08387692 | 0.12431649 | 0.06916996 |
| SSTR4 | | | | | | | | | |
| NMS)- | −0.1085645 | 0.11093897 | −0.1183417 | 0.03675889 | 0.20061932 | 0.02318841 | −0.1637897 | 0.14888011 | 0.17140975 |
| NMUR2 | | | | | | | | | |
| NMU)- | 0.17427991 | −0.0973568 | 0.11411432 | −0.3175994 | −0.0245759 | 0.275042 | −0.0765582 | −0.03083 | 0.14492754 |
| NMUR2 | | | | | | | | | |
| ARF1)- | −0.1097497 | −0.0118577 | 0.18090783 | 0.07812912 | 0.12451413 | −0.1100132 | −0.0936162 | −0.2326746 | −0.2096179 |
| CHRM3 | | | | | | | | | |
| VEGFC)- | 0.08210603 | 0.09218503 | 0.06709065 | −0.0552079 | −0.1324746 | 0.02272952 | −0.0610793 | −0.0890704 | 0.04433611 |
| LYVE1 | | | | | | | | | |
| PCSK9)- | 0.11901546 | −0.2572493 | 0.27042347 | 0.16523916 | −0.3215498 | −0.0647627 | −0.1054157 | 0.35401845 | −0.197635 |
| SORT1 | | | | | | | | | |
| LRPAP1)- | −0.0661615 | 0.24758642 | −0.0629408 | −0.4325713 | 0.01377038 | 0.65529168 | −0.0890792 | −0.1186469 | −0.1011266 |
| SORT1 | | | | | | | | | |
| BDNF)- | 0.22222224 | −0.1819949 | 0.00935935 | 0.06180608 | 0.04586491 | −0.1544442 | 0.17499012 | 0.14967062 | −0.116407 |
| SORT1 | | | | | | | | | |
| DLK1)- | −0.0983758 | −0.0193721 | 0.14327139 | −0.0974999 | −0.2364104 | 0.16304885 | −0.0091595 | 0.02925287 | −0.028594 |
| NOTCH2 | | | | | | | | | |
| DLL1)- | −0.3697648 | −0.2923952 | 0.2158856 | 0.03636483 | 0.06832707 | 0.12306071 | 0.33365179 | −0.1986825 | 0.15810277 |
| NOTCH2 | | | | | | | | | |
| MFNG)- | −0.0607159 | −0.0867559 | −0.1386279 | −0.1085639 | 0.05027178 | 0.02318841 | 0.10772525 | 0.08735178 | −0.0031621 |
| NOTCH2 | | | | | | | | | |
| PSEN1)- | −0.0397997 | −0.093437 | −0.0274162 | 0.11140758 | 0.00286637 | 0.06990151 | −0.1550372 | 0.10329721 | −0.1768833 |
| NOTCH2 | | | | | | | | | |
| ADAM10)- | −0.2506753 | −0.0411094 | 0.30731724 | −0.1152871 | 0.06793174 | −0.0793834 | −0.1792785 | 0.15111989 | −0.0048748 |
| NOTCH2 | | | | | | | | | |
| APP)- | −0.3264271 | 0.08162324 | 0.14077222 | 0.08722003 | 0.08433537 | 0.05783926 | 0.09039697 | −0.2140975 | 0.10935441 |
| NOTCH2 | | | | | | | | | |
| JAG2)- | 0.00678591 | −0.2247258 | 0.096534 | 0.03893537 | −0.0956742 | 0.26767244 | −0.047241 | −0.0488801 | 0.09802372 |
| NOTCH2 | | | | | | | | | |
| DSC2)- | 0.06913369 | −0.3548483 | −0.0725044 | −0.3894598 | −0.0024381 | 0.30675979 | −0.0294554 | 0.03630374 | −0.1251688 |
| DSG1 | | | | | | | | | |
| PDGFC)- | −0.2843496 | 0.3801054 | 0.40843215 | −0.1519154 | −0.2770183 | 0.09289762 | 0.30461133 | −0.3488487 | 0.34229249 |
| PDGFRA | | | | | | | | | |
| PDGFB)- | 0.15221552 | −0.0185924 | 0.1625186 | 0.13999144 | 0.02635133 | −0.2815588 | 0.08577075 | −0.4773199 | 0.20803689 |
| PDGFRA | | | | | | | | | |
| PDGFD)- | 0.15556434 | 0.03089897 | 0.0657509 | −0.034125 | −0.0919661 | 0.17284886 | 0.04123847 | 0.05079553 | 0.14466403 |
| PDGFRA | | | | | | | | | |
| PDGFA)- | −0.0111701 | 0.1626911 | 0.08763838 | −0.1568007 | 0.04618375 | 0.12515649 | 0.10935441 | 0.09935106 | 0.11238472 |
| PDGFRA | | | | | | | | | |
| FGF19)- | −0.3281389 | −0.0432233 | 0.05590165 | 0.03992095 | −0.2754985 | 0.30863994 | 0.09826991 | 0.04828881 | 0.27035573 |
| KLB | | | | | | | | | |
| FGF21)- | −0.2492832 | −0.1437517 | 0.27155528 | 0.40237154 | −0.0733564 | 0.08135973 | −0.0639336 | −0.1185849 | 0.45429691 |
| KLB | | | | | | | | | |
| LAMA1)- | −0.333575 | 0.33355299 | 0.12163053 | −0.0181854 | 0.03786081 | 0.2907222 | −0.0397971 | 0.03438962 | −0.1554677 |
| NT5E | | | | | | | | | |
| FN1)- NT5E | −0.1428289 | 0.33636364 | 0.00645714 | −0.5059289 | 0.04888977 | 0.60899928 | −0.0517889 | 0.02002767 | 0.3370224 |
| CALM1)- | −0.128467 | −0.0004611 | 0.21012058 | 0.07325428 | 0.05910064 | −0.3392734 | 0.14304069 | 0.13906914 | 0.08036891 |
| MIP | | | | | | | | | |
| SEMA7A)- | −0.0758764 | −0.2099298 | −0.0516116 | 0.09558945 | −0.1141097 | 0.11311681 | 0.15216318 | 0.09295431 | 0.26363636 |
| PLXNC1 | | | | | | | | | |
| LTA)- | 0.17699408 | −0.0300544 | 0.15422148 | 0.08631482 | −0.0751104 | −0.083726 | 0.15757218 | 0.01021249 | 0.11119895 |
| RIPK1 | | | | | | | | | |
| TNF)- | −0.1788929 | 0.00105485 | 6.60E−05 | −0.3286435 | −0.1458945 | 0.07793963 | 0.09760117 | 0.20919124 | 0.31185771 |
| RIPK1 | | | | | | | | | |
| CD14)- | 0.1875453 | −0.1257124 | 0.04132749 | 0.23807642 | 0.09931462 | −0.1234064 | 0.12205089 | −0.1501565 | 0.18972332 |
| RIPK1 | | | | | | | | | |
| CALR)- | 0.00783978 | 0.10125832 | 0.06779326 | 0.12582345 | 0.25369742 | 0.08353096 | −0.1592279 | −0.2092227 | −0.1475626 |
| HLA-F | | | | | | | | | |
| B2M)- | 0.17807497 | 0.23341459 | 0.600784 | 0.43241107 | −0.0667347 | 0.60724638 | −0.2239204 | 0.46192358 | 0.45072464 |
| HLA-F | | | | | | | | | |
| HMGB1)- | −0.1554985 | −0.1535218 | 0.20114231 | −0.0766798 | 0.02937008 | −0.1180501 | 0.04336212 | −0.0934124 | −0.1623188 |
| TLR2 | | | | | | | | | |
| CCN1)- | 0.35256917 | 0.49855072 | −0.345511 | −0.1171278 | 0.22129861 | 0.04018445 | −0.4042511 | 0.37496706 | 0.55968379 |
| TLR2 | | | | | | | | | |
| HSP90B1)- | 0.16304885 | 0.1561975 | −0.0060722 | −0.0959157 | 0.22545662 | 0.03438735 | −0.1159623 | −0.2583663 | −0.1432148 |
| TLR2 | | | | | | | | | |
| BGN)- | 0.33003953 | 0.37536232 | −0.2184606 | −0.3541502 | 0.05299815 | −0.2094862 | −0.2546287 | 0.42648221 | 0.46284585 |
| TLR2 | | | | | | | | | |
| VCAN)- | 0.26845417 | 0.32715175 | −0.1813085 | −0.097892 | 0.17516449 | −0.0534914 | −0.1757585 | 0.4115942 | 0.44953887 |
| TLR2 | | | | | | | | | |
| ZG16B)- | −0.1816565 | −0.1488436 | 0.07822568 | −0.0638999 | −0.0962943 | −0.0284585 | −0.0389427 | 0.1891429 | 0.18999934 |
| TLR2 | | | | | | | | | |
| HRAS)- | −0.0463216 | −0.1422594 | −0.0532084 | −0.2129328 | 0.21524787 | −0.2616859 | 0.15397843 | −0.1799736 | −0.1239789 |
| TLR2 | | | | | | | | | |
| RNASE2)- | −0.1834838 | −0.2159758 | 0.32662055 | 0.19617918 | −0.2579287 | 0.29380764 | −0.2327826 | 0.3312253 | 0.31528327 |
| TLR2 | | | | | | | | | |
| CCN6)- | 0.31863909 | 0.04873226 | 0.19004923 | −0.1365179 | 0.07412288 | 0.03966398 | −0.0909091 | −0.1325428 | −0.1388669 |
| SORL1 | | | | | | | | | |
| MDK)- | −0.1471673 | −0.1891963 | 0.06495389 | −0.2479578 | 0.09907773 | 0.13333333 | −0.0537567 | −0.1341283 | 0.29315854 |
| SORL1 | | | | | | | | | |
| LRPAP1)- | 0.08540363 | 0.0555519 | 0.12546953 | 0.04459962 | −0.2651602 | 0.07595771 | 0.2616687 | 0.25975823 | −0.1803748 |
| SORL1 | | | | | | | | | |
| APP)- | 0.0668665 | −0.2318258 | 0.1294509 | 0.22068511 | −0.2562582 | −0.0310935 | 0.13201581 | 0.11607378 | −0.1849802 |
| SORL1 | | | | | | | | | |
| L1CAM)- | 0.0787096 | −0.1960812 | 0.1310059 | 0.02819592 | 0.13476557 | −0.2267532 | −0.0188493 | 0.08011596 | 0.39135591 |
| ITGAV | | | | | | | | | |
| TNC)- | −0.2722241 | 0.39189458 | 0.28388266 | 0.34532279 | 0.28270183 | 0.34532279 | 0.00171334 | −0.070751 | −0.1915679 |
| ITGAV | | | | | | | | | |
| PLAU)- | −0.075775 | −0.1521431 | 0.14441552 | 0.45185942 | 0.11334806 | 0.27794064 | 0.18958815 | 0.22371542 | −0.0764163 |
| ITGAV | | | | | | | | | |
| EDIL3)- | −0.1254282 | 0.32542819 | 0.17416805 | −0.1477651 | 0.03347722 | 0.27075991 | 0.04685338 | −0.086166 | 0.2886693 |
| ITGAV | | | | | | | | | |
| COL4A5)- | 0.22240528 | 0.16744647 | −0.0128214 | −0.1555995 | 0.14398683 | −0.085639 | −0.1055719 | 0.23011298 | −0.0712803 |
| ITGAV | | | | | | | | | |
| SPP1)- | 0.07352021 | −0.0084324 | −0.0008567 | 0.30013175 | 0.30754532 | 0.10408432 | 0.05647447 | 0.07444005 | 0.08945982 |
| ITGAV | | | | | | | | | |
| NID1)- | 0.13478705 | −0.0407787 | 0.44321067 | 0.19433465 | −0.0727512 | 0.08036891 | 0.36579903 | 0.02450593 | −0.0007905 |
| ITGAV | | | | | | | | | |
| PDGFB)- | −0.0017801 | 0.00013186 | 0.06199506 | −0.0934813 | −0.3283799 | 0.0197635 | −0.0689292 | 0.31923584 | 0.03899868 |
| ITGAV | | | | | | | | | |
| COL4A4)- | 0.10143788 | 0.20017162 | 0.14630209 | 0.07073406 | −0.2110851 | −0.2039619 | 0.14293717 | 0.13142725 | 0.24368392 |
| ITGAV | | | | | | | | | |
| CALR)- | 0.16410831 | −0.1211542 | −0.0263279 | −0.0899868 | −0.1252059 | 0.27101449 | −0.0859308 | −0.0808959 | −0.4060606 |
| ITGAV | | | | | | | | | |
| CCN1)- | −0.1019763 | 0.17562582 | 0.37041188 | 0.23267457 | −0.2040198 | 0.06916996 | 0.34398684 | −0.0906456 | 0.09077734 |
| ITGAV | | | | | | | | | |
| FGG)- | 0.11029121 | 0.26518646 | 0.42223029 | −0.1751762 | 0.00863319 | 0.15442388 | −0.1183565 | 0.25198458 | 0.0084983 |
| ITGAV | | | | | | | | | |
| FGA)- | 0.04867435 | 0.19562077 | −0.0199908 | 0.05784117 | −0.044812 | −0.0798445 | −0.0697199 | 0.00513834 | 0.1088274 |
| ITGAV | | | | | | | | | |
| COL4A3)- | −0.2019384 | 0.25870261 | 0.35817454 | −0.3183765 | −0.275178 | −0.0176583 | −0.232883 | −0.2964427 | −0.1173913 |
| ITGAV | | | | | | | | | |
| MFGE8)- | 0.00289855 | −0.0716733 | 0.3543328 | 0.28440609 | −0.1284434 | 0.25219053 | 0.39551897 | −0.0731225 | 0.02608696 |
| ITGAV | | | | | | | | | |
| FGB)- | 0.03782911 | −0.0221439 | 0.20318423 | −0.0542322 | −0.1393494 | −0.181279 | −0.069428 | −0.3473878 | 0.09789841 |
| ITGAV | | | | | | | | | |
| AZGP1)- | 0.03320267 | 0.36338483 | 0.18531089 | 0.0689746 | 0.09140335 | −0.0117922 | −0.0267545 | −0.242556 | −0.34361 |
| ITGAV | | | | | | | | | |
| ADAM9)- | 0.13399209 | −0.3101449 | −0.1476112 | −0.3229249 | −0.1284349 | 0.24492754 | 0.11993411 | −0.0931489 | 0.44808959 |
| ITGAV | | | | | | | | | |
| VEGFA)- | −0.2509881 | 0.16152833 | 0.26082374 | −0.0075099 | 0.2158814 | −0.1786561 | −0.0508748 | 0.47471919 | 0.00724662 |
| ITGAV | | | | | | | | | |
| ADAM15)- | 0.18498024 | 0.06324111 | −0.006458 | −0.0201581 | −0.2002636 | 0.16284585 | −0.2029654 | 0.27905138 | −0.173386 |
| ITGAV | | | | | | | | | |
| COL1A2)- | −0.2466403 | 0.20263505 | 0.28685339 | 0.17628458 | −0.0492916 | 0.63570487 | 0.30767712 | −0.0479578 | 0.25362319 |
| ITGAV | | | | | | | | | |
| FN1)- | −0.2349144 | 0.00316206 | 0.31380562 | 0.38629776 | −0.0142998 | 0.49183136 | 0.27196048 | −0.0931489 | −0.0239789 |
| ITGAV | | | | | | | | | |
| CP)- | 0.23617379 | −0.0559307 | 0.30356731 | −0.1050066 | −0.1803689 | −0.1238472 | −0.057907 | −0.0206199 | −0.1246418 |
| SLC40A1 | | | | | | | | | |
| SOSTDC1)- | 0.01087207 | 0.07197944 | −0.1200119 | 0.10636616 | −0.1529338 | −0.2426299 | 0.33675958 | −0.0885375 | 0.07855284 |
| LRP6 | | | | | | | | | |
| IGFBP4)- | 0.10974967 | −0.3296979 | 0.13639059 | 0.05047943 | 0.03584371 | −0.2530962 | 0.07623378 | 0.04110672 | −0.3235033 |
| LRP6 | | | | | | | | | |
| PTH)- LRP6 | 0.03217301 | 0.18591923 | −0.204583 | 0.09055261 | 0.00625988 | 0.24481191 | −0.2311392 | 0.26034256 | 0.04790932 |
| RSPO3)- | 0.05494614 | −0.2070784 | −0.0543969 | 0.01489439 | −0.0199657 | −0.1889453 | 0.01186005 | 0.15533597 | 0.11460016 |
| LRP6 | | | | | | | | | |
| WNT2)- | −0.1152668 | −0.1088146 | 0.24494909 | 0.33753997 | −0.1577387 | 0.03662714 | 0.05297664 | −0.0214763 | 0.08830895 |
| LRP6 | | | | | | | | | |
| CCN2)- | 0.15349144 | 0.03532242 | 0.00823615 | 0.09779565 | −0.1300652 | −0.0981555 | 0.12044541 | 0.18498024 | −0.0123233 |
| LRP6 | | | | | | | | | |
| SOST)- | −0.2349881 | 0.14770557 | −0.2763324 | −0.0832345 | −0.0399302 | 0.03386146 | −0.1574092 | 0.03003953 | 0.09680715 |
| LRP6 | | | | | | | | | |
| APP)- LRP6 | 0.09216377 | −0.3406814 | 0.13076138 | −0.0760486 | −0.1543124 | −0.1101449 | 0.20326811 | 0.13570487 | 0.10313355 |
| CKLF)- | −0.084171 | 0.25708823 | 0.31374232 | 0.131409 | 0.05653477 | 0.06890873 | −0.2242941 | 0.03715537 | 0.09166997 |
| LRP6 | | | | | | | | | |
| WNT5A)- | −0.1942394 | 0.07163815 | 0.25776256 | 0.17188994 | −0.2055354 | 0.15469759 | 0.16327338 | 0.02819499 | −0.1791822 |
| LRP6 | | | | | | | | | |
| DKK1)- | −0.1761218 | −0.1931912 | 0.03571899 | 0.02372479 | −0.0048101 | 0.20244409 | 0.03096791 | −0.0326746 | −0.1948664 |
| LRP6 | | | | | | | | | |
| WNT7A)- | 0.03914333 | −0.0400475 | 0.00201028 | −0.0450442 | 0.06971304 | −0.0214763 | −0.0467154 | −0.1642951 | 0.09568685 |
| LRP6 | | | | | | | | | |
| DKK2)- | −0.1014402 | 0.08146512 | 0.21584206 | −0.1027086 | −0.0526472 | −0.0688428 | 0.3469724 | −0.0183136 | 0.32699597 |
| LRP6 | | | | | | | | | |
| DSC2)- | 0.21066852 | −0.1356792 | 0.26313016 | −0.2051383 | 0.20409104 | 0.13346509 | −0.4145859 | −0.4291973 | 0.26265687 |
| DSG2 | | | | | | | | | |
| PDGFC)- | −0.1068511 | 0.26284585 | 0.29011858 | 0.02747126 | 0.03116045 | −0.0565236 | 0.33768116 | −0.1957839 | 0.3798419 |
| PDGFRB | | | | | | | | | |
| PDGFB)- | 0.22581185 | 0.01826274 | 0.2614143 | 0.03827531 | −0.1006621 | −0.2656873 | −0.1250329 | −0.18722 | 0.03833992 |
| PDGFRB | | | | | | | | | |
| PDGFD)- | −0.0390684 | 0.03926607 | −0.0723392 | 0.13307421 | 0.12905563 | 0.08979215 | 0.07088274 | −0.2258235 | 0.2599473 |
| PDGFRB | | | | | | | | | |
| MFGE8)- | −0.1035573 | 0.38458498 | 0.33992095 | −0.2657619 | −0.2163515 | −0.008828 | 0.34927536 | −0.1216074 | 0.4516469 |
| PDGFRB | | | | | | | | | |
| PDGFA)- | −0.1721798 | 0.05113337 | −0.0407222 | −0.0948052 | 0.03979313 | 0.20311625 | 0.01765481 | 0.13083004 | 0.02450593 |
| PDGFRB | | | | | | | | | |
| PDGFC)- | −0.0256951 | 0.08168643 | 0.13137869 | 0.11956916 | −0.3369572 | 0.03129633 | −0.0486905 | 0.20780077 | 0.22924901 |
| FLT4 | | | | | | | | | |
| VEGFC)- | 0.04270322 | −0.0133759 | 0.11051799 | 0.01554728 | 0.18211768 | −0.0105419 | −0.0030967 | −0.0429569 | 0.04519104 |
| FLT4 | | | | | | | | | |
| TNF)- FLT4 | 0.05644205 | 0.06777428 | −0.0849956 | 0.03782662 | −0.2500989 | 0.30772781 | 0.00665459 | 0.25708262 | 0.37575758 |
| COL1A2)- | −0.0463829 | 0.02700922 | −0.008763 | 0.16482213 | −0.1445561 | 0.21346686 | −0.0805139 | 0.13480037 | 0.11607378 |
| FLT4 | | | | | | | | | |
| FN1)- FLT4 | −0.0280669 | −0.0048748 | −0.0376215 | 0.11725955 | −0.0934937 | 0.27342206 | −0.0224675 | 0.16141784 | 0.04637681 |
| LGALS3BP)- | 0.09617918 | 0.41027668 | −0.1373493 | 0.36034256 | −0.1799249 | 0.19591568 | 0.24728138 | −0.2957839 | −0.3104084 |
| CD33 | | | | | | | | | |
| IL12A)- | −0.1608927 | −0.1281688 | 0.20932841 | 0.16451443 | −0.1867028 | 0.2161539 | −0.140126 | −0.0032281 | 0.14303597 |
| IL12RB2 | | | | | | | | | |
| F2)- | 0.02781531 | 0.12387573 | 0.23193516 | 0.23644509 | −0.1957897 | 0.05654409 | −0.1357602 | −0.0380902 | 0.30409434 |
| ITGA2B | | | | | | | | | |
| CALR)- | −0.209602 | 0.23993676 | 0.06852474 | 0.3831357 | −0.0786665 | −0.1719934 | −0.0790618 | −0.011598 | −0.0397892 |
| ITGA2B | | | | | | | | | |
| FGG)- | −0.1736637 | 0.1185927 | −0.0824987 | 0.13505501 | 0.19226461 | 0.13444049 | 0.06443749 | −0.1246829 | 0.09110972 |
| ITGA2B | | | | | | | | | |
| FGA)- | −0.0158673 | −0.0401003 | −0.0206464 | 0.1367634 | −0.0899358 | 0.08257274 | 0.00467782 | −0.307084 | −0.0247694 |
| ITGA2B | | | | | | | | | |
| FGB)- | −0.152586 | 0.09516592 | −0.0418218 | −0.0689269 | 0.04544107 | −0.1119937 | −0.0662516 | −0.2186635 | −0.2322287 |
| ITGA2B | | | | | | | | | |
| COL1A2)- | −0.163888 | −0.1239789 | −0.2113585 | 0.33399209 | 0.10567927 | −0.1389127 | −0.252866 | −0.119341 | 0.10184453 |
| ITGA2B | | | | | | | | | |
| FN1)- | −0.0632619 | −0.0674572 | −0.1344051 | 0.29749671 | −0.0283305 | 0.00856672 | −0.1803268 | −0.0073806 | 0.05507246 |
| ITGA2B | | | | | | | | | |
| GCG)- | −0.0858564 | −0.0343271 | 0.08322077 | −0.1506127 | 0.00039534 | 0.17638532 | −0.0761578 | 0.1600896 | 0.16284585 |
| GLP2R | | | | | | | | | |
| CALM1)- | 0.14451362 | 0.11205903 | −0.0944099 | 0.0942029 | −0.0429541 | 0.23690625 | 0.08907043 | −0.1462547 | 0.19525692 |
| GLP2R | | | | | | | | | |
| CALM1)- | 0.19540154 | −0.2363055 | 0.25363154 | 0.085639 | −0.326087 | −0.1430877 | 0.45362319 | 0.31338318 | −0.3789196 |
| KCNN4 | | | | | | | | | |
| CALR)- | −0.0596317 | 0.26629772 | −0.0125569 | 0.34856221 | −0.0018455 | −0.1728875 | 0.05431058 | −0.0970516 | 0.20303699 |
| SCARF1 | | | | | | | | | |
| ARF1)- | −0.194934 | −0.177608 | 0.37848734 | −0.1406502 | 0.40902222 | −0.270167 | −0.1671834 | 0.15929378 | −0.1026384 |
| PLD2 | | | | | | | | | |
| HRAS)- | −0.0360838 | 0.20908702 | −0.2574856 | −0.1989458 | 0.01883309 | −0.209028 | 0.02206888 | −0.0207558 | −0.390869 |
| GRIN2D | | | | | | | | | |
| HSPA1A)- | −0.0873777 | 0.06100936 | −0.1175873 | 0.3286119 | 0.03118716 | 0.09244556 | 0.01499891 | 0.08467038 | −0.3326965 |
| GRIN2D | | | | | | | | | |
| IL27)- | 0.08586874 | 0.17589879 | −0.0567301 | 0.00349155 | 0.13052658 | −0.1151933 | 0.147407 | 0.10003625 | 0.12780395 |
| IL27RA | | | | | | | | | |
| DLK1)- | −0.1945113 | 0.01812012 | −0.0383171 | 0.01534965 | 0.1009948 | 0.11390362 | 0.13974634 | −0.2083278 | 0.06469891 |
| NOTCH3 | | | | | | | | | |
| DLL1)- | −0.2977379 | 0.19194 | 0.07097397 | −0.023255 | 0.04624811 | −0.0038868 | 0.25488323 | −0.1602108 | 0.38418972 |
| NOTCH3 | | | | | | | | | |
| PSEN1)- | −0.1740907 | 0.18054824 | 0.16421206 | 0.14724775 | 0.00513902 | −0.1870409 | −0.307069 | 0.15171778 | −0.2678613 |
| NOTCH3 | | | | | | | | | |
| THBS2)- | 0.18511199 | −0.0334651 | 0.13426002 | 0.29841897 | 0.17773971 | −0.5358366 | 0.133733 | 0.16666667 | −0.0549407 |
| NOTCH3 | | | | | | | | | |
| SCGB3A1)- | 0.04923382 | 0.01403856 | −0.0502241 | 0.13669752 | 0.10547467 | −0.2005995 | 0.15653205 | −0.2801805 | 0.18696268 |
| NOTCH3 | | | | | | | | | |
| JAG2)- | 0.00632474 | −0.194815 | −0.2166293 | −0.481257 | −0.3022038 | 0.3076619 | 0.34408248 | −0.2247694 | 0.38906456 |
| NOTCH3 | | | | | | | | | |
| CLCF1)- | −0.4237573 | 0.12583589 | 0.22556938 | −0.0973055 | −0.149298 | 0.10251005 | 0.25933497 | −0.1715472 | 0.11667051 |
| LIFR | | | | | | | | | |
| CNTF)- | −0.288412 | −0.1456913 | 0.21267487 | 0.23091113 | −0.1996573 | 0.15317215 | −0.2930399 | 0.15849802 | 0.31185771 |
| LIFR | | | | | | | | | |
| APP)- | 0.11390362 | 0.10204552 | 0.10402187 | −0.2104084 | 0.06824769 | 0.18076416 | −0.0212121 | −0.0364954 | 0.48537549 |
| APLP2 | | | | | | | | | |
| PCSK9)- | −0.0477116 | −0.0755214 | 0.05061123 | 0.31709326 | −0.347004 | −0.3440393 | 0.24729908 | 0.314361 | −0.5335968 |
| APLP2 | | | | | | | | | |
| BMP7)- | −0.0046156 | 0.00408715 | 0.09058811 | −0.0616702 | −0.0307079 | −0.0763477 | 0.05086092 | −0.1735864 | 0.01258442 |
| ACVR1 | | | | | | | | | |
| INHBB)- | −0.048107 | 0.35617341 | −0.0147762 | 0.36434313 | 0.00587091 | −0.1248147 | −0.2124083 | −0.1304162 | 0.15430228 |
| ACVR1 | | | | | | | | | |
| BMP2)- | −0.1444953 | 0.00658892 | 0.28894703 | 0.16563993 | 0.09858838 | 0.03716884 | −0.1361703 | 0.02359532 | −0.1212441 |
| ACVR1 | | | | | | | | | |
| BMP6)- | −0.1567322 | 0.01081117 | −0.000495 | 0.12928308 | 0.24219052 | −0.2073818 | 0.07084906 | −0.1373072 | 0.36587053 |
| ACVR1 | | | | | | | | | |
| TGFB2)- | 0.01627999 | 0.01423347 | −0.0320314 | 0.01910912 | 0.01332675 | −0.1752513 | −0.3435256 | −0.0021748 | 0.13328941 |
| ACVR1 | | | | | | | | | |
| GPC3)- | −0.0831905 | −0.0152933 | −0.2137817 | −0.2471673 | −0.179374 | 0.26007905 | 0.0554201 | 0.05072464 | 0.09670619 |
| CD81 | | | | | | | | | |
| CD99)- | 0.06982872 | 0.04018445 | 0.14016475 | −0.0934155 | 0.16557383 | −0.2982312 | 0.00856672 | 0.14980237 | −0.0252964 |
| CD81 | | | | | | | | | |
| VCAN)- | −0.2275587 | −0.16536 | 0.03689794 | 0.01699661 | 0.17479822 | 0.04927861 | −0.063581 | −0.1892747 | −0.0221351 |
| SELP | | | | | | | | | |
| CD34)- | −0.0699315 | −0.0727962 | 0.05764477 | 0.31741221 | −0.1247941 | 0.24172349 | 0.23086806 | 0.10534291 | 0.27530551 |
| SELP | | | | | | | | | |
| SERPING1)- | 0.09308914 | 0.24869067 | 0.12057322 | 0.13379887 | −0.1512766 | −0.0166678 | 0.06483281 | 0.1816984 | 0.28808591 |
| SELP | | | | | | | | | |
| CD24)- | −0.0863693 | 0.14974143 | 0.24200297 | 0.21463158 | 0.2165047 | −0.06285 | −0.0992258 | −0.1604849 | −0.0003953 |
| SELP | | | | | | | | | |
| GAS6)- | −0.1029087 | −0.1890646 | 0.10193391 | −0.0304348 | −0.1721082 | −0.0740521 | 0.11590288 | 0.00026353 | −0.1479578 |
| TYRO3 | | | | | | | | | |
| PROS1)- | 0.19434012 | −0.1987021 | 0.06912685 | 0.18484848 | 0.06971305 | −0.2158975 | −0.2402399 | 0.39490068 | −0.2243742 |
| TYRO3 | | | | | | | | | |
| VEGFA)- | −0.0711533 | −0.0681159 | −0.02181 | −0.0088274 | −0.07762 | 0.01950127 | 0.36804824 | −0.1222164 | 0.03386146 |
| TYRO3 | | | | | | | | | |
| PDGFC)- | 0.11897233 | 0.07285903 | −0.0742767 | 0.04940874 | 0.11603231 | −0.1969103 | 0.08376723 | −0.1048748 | −0.1714097 |
| KDR | | | | | | | | | |
| VEGFC)- | 0.17770896 | 0.13435246 | 0.02214971 | −0.1144965 | −0.25276 | 0.11752693 | 0.15725303 | −0.1395257 | −0.0693017 |
| KDR | | | | | | | | | |
| GREM1)- | −0.1475626 | −0.1156785 | 0.16924802 | −0.1089592 | 0.28359589 | −0.1916996 | 0.03460094 | −0.1052701 | 0.03689065 |
| KDR | | | | | | | | | |
| TIMP3)- | −0.0209486 | 0.12859025 | −0.169248 | −0.002108 | 0.12891321 | −0.1366271 | −0.1343835 | −0.0048748 | 0.09090909 |
| KDR | | | | | | | | | |
| CXCL8)- | −0.1685223 | −0.0975031 | 0.21180466 | 0.14571805 | 0.00382258 | −0.1314888 | 0.1418309 | 0.04466403 | 0.07470356 |
| KDR | | | | | | | | | |
| COL18A1)- | −0.3100597 | 0.381693 | 0.14089336 | −0.262591 | 0.30911191 | −0.0193682 | 0.03868715 | −0.2754941 | 0.03201581 |
| KDR | | | | | | | | | |
| VEGFA)- | −0.112253 | 0.28247694 | 0.075463 | 0.41330698 | −0.001384 | −0.2350461 | 0.21990445 | 0.09835634 | −0.1224019 |
| KDR | | | | | | | | | |
| TNFSF12)- | −0.0266465 | 0.14358274 | 0.14639543 | −0.1249094 | 0.0035626 | 0.10333696 | −0.1090844 | 0.19349101 | −0.0239138 |
| TNFRSF8 | | | | | | | | | |
| AFDN)- | −0.0370895 | −0.0115946 | −0.2369262 | −0.0399209 | −0.1821537 | −0.0072464 | 0.3132435 | 0.10105735 | −0.1649593 |
| NECTIN1 | | | | | | | | | |
| WNT5A)- | 0.01799427 | −0.3025969 | −0.0456463 | 0.16840164 | −0.0819661 | 0.00540273 | 0.03010739 | 0.00507263 | −0.0252964 |
| MCAM | | | | | | | | | |
| LAMA1)- | −0.0228029 | −0.1274381 | 0.23972852 | −0.1048297 | −0.0254341 | 0.06998583 | −0.18189 | −0.0884862 | −0.1135705 |
| GPC1 | | | | | | | | | |
| TDGF1)- | −0.1647976 | 0.29596674 | 0.07292222 | −0.1019138 | −0.0083009 | 0.1180734 | −0.0173919 | 0.09955526 | −0.2051383 |
| GPC1 | | | | | | | | | |
| BMP2)- | −0.1464106 | −0.0057975 | 0.33909807 | −0.203564 | −0.1208248 | 0.03538249 | −0.1582606 | −0.0379559 | 0.00843326 |
| GPC1 | | | | | | | | | |
| NRG1)- | 0.12731233 | −0.201965 | 0.12198745 | 0.08188945 | −0.1443819 | −0.0421705 | −0.0823479 | 0.07689013 | 0.02463768 |
| GPC1 | | | | | | | | | |
| APP)- | −0.03983 | 0.28143219 | 0.27294288 | −0.1379447 | −0.2139728 | 0.32278044 | 0.2033005 | −0.0424312 | 0.26996047 |
| GPC1 | | | | | | | | | |
| SERPINC1)- | −0.242804 | −0.0112085 | −0.1459137 | −0.0883574 | 0.15168188 | 0.34650236 | 0.03102971 | 0.07583844 | 0.1090945 |
| GPC1 | | | | | | | | | |
| COL18A1)- | −0.1699183 | 0.40818481 | 0.22485832 | −0.0208834 | −0.1785032 | 0.39111814 | 0.29098455 | −0.1225498 | 0.26350461 |
| GPC1 | | | | | | | | | |
| SLIT2)- | 0.13514315 | 0.12458001 | 0.31850973 | −0.0225296 | −0.1221384 | −0.145215 | 0.18801673 | 0.3209356 | 0.1201581 |
| GPC1 | | | | | | | | | |
| SHH)- | 0.0096873 | −0.0832839 | −0.0687247 | 0.28089592 | 0.19941368 | −0.1508154 | −0.1626536 | −0.0745841 | −0.0642951 |
| GPC1 | | | | | | | | | |
| VEGFA)- | −0.2853566 | 0.49591568 | 0.43947429 | 0.44993412 | 0.19216707 | −0.2357437 | −0.0735226 | 0.20959347 | −0.2851214 |
| GPC1 | | | | | | | | | |
| CD274)- | −0.0318297 | −0.0479768 | 0.27514346 | 0.15066869 | −0.038634 | 0.09914688 | 0.02231673 | −0.0131098 | 0.15719086 |
| CD80 | | | | | | | | | |
| IL18)- | 0.03874028 | −0.0440111 | 0.07005744 | 0.09301713 | 0.04979887 | 0.18221344 | −0.0715676 | −0.0613986 | 0.01014526 |
| IL18RAP | | | | | | | | | |
| RTN4)- | −0.1150069 | −0.2324111 | −0.134353 | −0.2432228 | −0.2695719 | 0.03664525 | 0.05607016 | −0.1464162 | −0.1706249 |
| CNTNAP1 | | | | | | | | | |
| TNC)- | 0.10728172 | 0.05990313 | −0.186258 | 0.11555058 | 0.24351837 | −0.2187088 | 0.06226322 | −0.0628458 | 0.257716 |
| ITGA9 | | | | | | | | | |
| VEGFC)- | 0.02411623 | −0.0426331 | −0.2203111 | 0.19171223 | 0.09843843 | 0.17945255 | 0.22111679 | −0.2022398 | 0.21496097 |
| ITGA9 | | | | | | | | | |
| SPP1)- | −0.1057347 | −0.1537651 | 0.0785399 | 0.15389176 | 0.31579641 | −0.1699605 | 0.08479658 | −0.0646904 | 0.18307586 |
| ITGA9 | | | | | | | | | |
| F13A1)- | −0.194288 | 0.07234155 | 0.1915195 | −0.0111334 | −0.1778949 | 0.04782609 | 0.03781914 | −0.1306983 | 0.13241543 |
| ITGA9 | | | | | | | | | |
| ADAM12)- | 0.14929995 | 0.08473348 | −0.0289951 | 0.13894654 | 0.26672817 | −0.41676 | 0.1085159 | −0.0932806 | 0.2637768 |
| ITGA9 | | | | | | | | | |
| VCAM1)- | −0.2520178 | 0.44073269 | 0.40237232 | 0.15501169 | 0.01409982 | −0.0284585 | 0.28311646 | −0.2242424 | 0.45423104 |
| ITGA9 | | | | | | | | | |
| TGM2)- | −0.180309 | 0.21496805 | 0.04276207 | −0.1553462 | −0.4152995 | 0.42386113 | 0.39426784 | −0.4602108 | 0.35455713 |
| ITGA9 | | | | | | | | | |
| VEGFA)- | −0.3621871 | 0.22345927 | 0.15292374 | −0.012978 | 0.15575688 | −0.1768116 | −0.1393556 | 0.06647123 | −0.042427 |
| ITGA9 | | | | | | | | | |
| CSF2)- | −0.2042122 | −0.0019117 | −0.0471058 | −0.2232508 | −0.1253624 | 0.42514083 | 0.00032944 | −0.1105402 | 0.01218749 |
| ITGA9 | | | | | | | | | |
| ADAM15)- | −0.3924901 | 0.31199974 | 0.07577006 | 0.21028361 | 0.26190084 | −0.4853755 | 0.01093724 | −0.173913 | 0.21331401 |
| ITGA9 | | | | | | | | | |
| FN1)- | −0.0137022 | −0.0818868 | 0.0137045 | 0.17253533 | 0.31540109 | −0.4761528 | 0.08585077 | −0.1346509 | 0.07391548 |
| ITGA9 | | | | | | | | | |
| NPPC)- | −0.1889344 | 0.2229624 | −0.0080454 | 0.13276669 | −0.0118601 | −0.0038875 | −0.1312253 | −0.0665349 | 0.17470356 |
| NPR3 | | | | | | | | | |
| VEGFB)- | 0.11609673 | −0.0428868 | 0.10278711 | −0.0220041 | 0.10924785 | −0.0977169 | −0.0562675 | 0.03663317 | −0.1339921 |
| NRP1 | | | | | | | | | |
| SEMA3D)- | −0.1665513 | 0.0986491 | −0.1972648 | −0.1301713 | −0.0660847 | −0.0849942 | 0.13428214 | 0.15180866 | −0.1726671 |
| NRP1 | | | | | | | | | |
| SEMA3B)- | −0.1107048 | −0.021149 | 0.21963033 | 0.10342897 | −0.1024577 | −0.1505568 | 0.00309669 | −0.1624115 | 0.12397892 |
| NRP1 | | | | | | | | | |
| PGF)- | −0.058746 | 0.29035446 | 0.41982142 | 0.04716733 | −0.0311646 | −0.002306 | 0.15470269 | −0.3741064 | 0.21501976 |
| NRP1 | | | | | | | | | |
| SEMA3E)- | −0.0224764 | 0.03525768 | 0.05879445 | 0.0604763 | −0.207485 | −0.0181525 | 0.02035908 | −0.0905287 | 0.3201581 |
| NRP1 | | | | | | | | | |
| SEMA4A)- | −0.128707 | 0.20815076 | −0.0090945 | 0.06916996 | 0.03920277 | −0.1163565 | 0.10686872 | 0.28930984 | −0.0052701 |
| NRP1 | | | | | | | | | |
| VEGFA)- | −0.1618844 | 0.25191041 | 0.20912535 | 0.29262187 | 0.20464504 | −0.1064734 | −0.1060816 | −0.1981287 | −0.1335354 |
| NRP1 | | | | | | | | | |
| FGF7)- | −0.0062601 | 0.07655818 | 0.07624131 | −0.1374177 | −0.2476693 | 0.05014001 | 0.11602702 | −0.0295174 | 0.07852437 |
| NRP1 | | | | | | | | | |
| NLGN2)- | −0.0021743 | 0.14686213 | 0.08205095 | −0.0211504 | −0.3877612 | 0.30572577 | 0.03301374 | 0.00362331 | 0.34454363 |
| NRXN2 | | | | | | | | | |
| AFDN)- | −0.2357204 | 0.00415047 | 0.09301483 | 0.33420073 | 0.01621034 | −0.1825488 | 0.15202636 | 0.18604651 | −0.0935503 |
| NRXN2 | | | | | | | | | |
| CALM2)- | 0.08209521 | −0.0673254 | −0.0604823 | −0.1235837 | 0.04572407 | 0.02602537 | 0.10146264 | 0.09283479 | 0.23530962 |
| CACNA1C | | | | | | | | | |
| NCAM1)- | −0.1557723 | 0.04012254 | −0.0747867 | 0.04598458 | 0.20774857 | −0.1169901 | −0.1405982 | −0.1254489 | 0.24598155 |
| CACNA1C | | | | | | | | | |
| CALM1)- | −0.0693813 | 0.09328371 | 0.07814199 | 0.45335968 | −0.1384899 | 0.02174271 | 0.12676242 | −0.018646 | −0.2462451 |
| CACNA1C | | | | | | | | | |
| CALM3)- | 0.01106938 | 0.23617379 | −0.2469445 | 0.48458498 | −0.2784293 | 0.0628562 | 0.08130189 | −0.0140998 | −0.2652174 |
| CACNA1C | | | | | | | | | |
| RSPO3)- | −0.0913943 | 0.05003953 | 0.08003295 | −0.2016668 | −0.1378867 | −0.2565809 | 0.01667051 | −0.1483116 | 0.4327547 |
| LGR5 | | | | | | | | | |
| VCAM1)- | −0.1439678 | 0.11985241 | −0.1455306 | −0.0730591 | 0.03854263 | −0.0245726 | −0.2748715 | −0.1227313 | 0.03715537 |
| ITGB7 | | | | | | | | | |
| FN1)- | −0.0616621 | 0.09097796 | −0.2571485 | −0.0847195 | 0.12590592 | 0.06772292 | −0.3061009 | 0.03643071 | 0.09302019 |
| ITGB7 | | | | | | | | | |
| NRG3)- | −0.1969872 | 0.10594327 | −0.0925304 | −0.2306425 | 0.34996212 | 0.38062605 | −0.0605422 | −0.1108037 | 0.16113307 |
| ERBB3 | | | | | | | | | |
| NRG1)- | −0.0805091 | −0.1047079 | 0.03666218 | −0.0066539 | −0.131963 | −0.0148231 | 0.21443394 | 0.36693017 | −0.0577075 |
| ERBB3 | | | | | | | | | |
| AREG)- | 0.17443848 | 0.000198 | −0.0430335 | 0.12898551 | −0.1149577 | −0.0023715 | 0.23921207 | 0.11298132 | −0.2753055 |
| ERBB3 | | | | | | | | | |
| L1CAM)- | −0.0300851 | 0.2211522 | 0.05179302 | 0.13017557 | −0.3672837 | −0.089858 | 0.05718992 | 0.11819739 | −0.395309 |
| ERBB3 | | | | | | | | | |
| TGFA)- | 0.03343888 | −0.0382536 | 0.16258297 | −0.0806324 | −0.0418986 | 0.08695652 | 0.17457756 | 0.25230567 | −0.3032938 |
| ERBB3 | | | | | | | | | |
| BTC)- | 0.3893374 | −0.2525948 | 0.34130752 | 0.19308936 | −0.284999 | −0.2795876 | −0.0669324 | −0.1063241 | 0.01581028 |
| ERBB3 | | | | | | | | | |
| SST)- | 0.22261764 | 0.22917766 | −0.2232187 | 0.15435799 | −0.0536674 | 0.05238189 | 0.10218548 | 0.09408354 | 0.29235837 |
| SSTR1 | | | | | | | | | |
| GPC3)- | −0.0780488 | −0.0798945 | 0.241759 | −0.1289855 | −0.3611807 | −0.1511199 | −0.0281328 | 0.19907773 | 0.20434783 |
| IGF1R | | | | | | | | | |
| INS)- | 0.03162472 | −0.15562 | 0.05245124 | −0.1214137 | −0.2460221 | −0.2741197 | −0.1362498 | 0.12819499 | −0.2094862 |
| IGF1R | | | | | | | | | |
| IGF2)- | 0.07912247 | −0.0990184 | 0.1697964 | 0.31462451 | 0.3110423 | 0.1259552 | −0.0260245 | 0.17114625 | 0.14545455 |
| IGF1R | | | | | | | | | |
| GNAI2)- | 0.07517955 | 0.07880345 | 0.14164745 | −0.0687747 | 0.04783239 | 0.03504611 | 0.25049414 | 0.08234519 | −0.0459816 |
| IGF1R | | | | | | | | | |
| IGF1)- | −0.1076447 | −0.001649 | 0.0348638 | −0.1789363 | −0.0338804 | 0.09490543 | −0.0648307 | −0.102635 | 0.03320158 |
| IGF1R | | | | | | | | | |
| HSP90B1)- | −0.1778715 | −0.116934 | −0.1024889 | −0.0704875 | 0.10423263 | 0.15836627 | 0.0095596 | −0.3051383 | −0.342556 |
| ASGR1 | | | | | | | | | |
| S100A4)- | −0.0822847 | 0.05705251 | −0.2346004 | 0.06785244 | 0.01725955 | 0.11422925 | 0.128722 | −0.0130435 | −0.158498 |
| ERBB2 | | | | | | | | | |
| NRG3)- | −0.0379075 | 0.02683193 | 0.05076311 | 0.08118617 | −0.0114662 | 0.15927513 | −0.2196311 | −0.2700922 | 0.31054018 |
| ERBB2 | | | | | | | | | |
| SEMA4D)- | −0.1128097 | 0.20809173 | −0.15597 | −0.1060606 | 0.15507246 | 0.21277997 | 0.07233202 | −0.014888 | −0.1403162 |
| ERBB2 | | | | | | | | | |
| NRG1)- | −0.2041409 | −0.2311751 | 0.00613214 | −0.3956124 | 0.00408459 | 0.12135187 | −0.0749671 | 0.00092227 | 0.03570487 |
| ERBB2 | | | | | | | | | |
| NRG4)- | 0.20474625 | 0.10177985 | 0.29584713 | −0.2039001 | −0.0685157 | 0.00052704 | −0.1562582 | −0.0557312 | −0.1454545 |
| ERBB2 | | | | | | | | | |
| HSP90B1)- | 0.12292895 | −0.1801772 | 0.12180902 | 0.06192358 | −0.0517787 | −0.1172596 | 0.1259552 | 0.15928854 | −0.2275362 |
| ERBB2 | | | | | | | | | |
| L1CAM)- | 0.00639969 | −0.0917729 | −0.1410571 | −0.1690438 | −0.0724003 | 0.12543233 | 0.02292792 | −0.036632 | 0.19040717 |
| ERBB2 | | | | | | | | | |
| TGFA)- | 0.05526979 | −0.28644 | 0.06865854 | −0.3969697 | 0.16864295 | 0.15006588 | 0.13899868 | 0.09578393 | 0.16403162 |
| ERBB2 | | | | | | | | | |
| HSP90AA1)- | 0.02700922 | 0.24044796 | 0.02384717 | 0.2801054 | −0.0258235 | 0.02239789 | 0.04980237 | −0.0144928 | −0.0581028 |
| ERBB2 | | | | | | | | | |
| BTC)- | −0.0273485 | 0.30136086 | 0.12494646 | 0.1720083 | −0.2267532 | −0.1334036 | −0.1666667 | −0.1235837 | −0.2559947 |
| ERBB2 | | | | | | | | | |
| IFNA16)- | −0.1245426 | −0.1382653 | −0.1684197 | 0.12352592 | 0.06279238 | 0.17470356 | 0.03017824 | −0.0874864 | −0.2018974 |
| IFNAR1 | | | | | | | | | |
| IFNA14)- | −0.15743 | 0.10692633 | 0.00112049 | −0.1514532 | 0.14554084 | 0.11506525 | 0.15964947 | −0.0913043 | −0.0899269 |
| IFNAR1 | | | | | | | | | |
| IFNA4)- | 0.03931791 | −0.0179779 | −0.1552243 | 0.01634375 | 0.38768785 | −0.2421087 | 0.26586282 | −0.2036891 | −0.251927 |
| IFNAR1 | | | | | | | | | |
| IFNA13)- | 0.06402059 | 0.26526449 | 0.04378793 | −0.1220107 | 0.00507347 | 0.01027668 | −0.0599591 | −0.2027668 | 0.02747217 |
| IFNAR1 | | | | | | | | | |
| IFNB1)- | −0.2879647 | −0.0122932 | −0.0531347 | 0.00882828 | 0.05811617 | 0.28340854 | 0.05066878 | −0.0486166 | 0.04585282 |
| IFNAR1 | | | | | | | | | |
| IFNA8)- | −0.0290209 | 0.23290804 | −0.1516971 | −0.206608 | 0.13118967 | −0.1073158 | 0.23667392 | −0.2776021 | −0.2039001 |
| IFNAR1 | | | | | | | | | |
| IFNE)- | 0.38401216 | −0.0876557 | −0.3902511 | −0.0437446 | −0.1840944 | 0.04874835 | 0.24333674 | −0.0899239 | 0.24673057 |
| IFNAR1 | | | | | | | | | |
| MMP9)- | 0.1838308 | −0.0932393 | −0.0545011 | 0.00355813 | 0.19486639 | −0.2352825 | 0.24148383 | −0.2137022 | −0.1857171 |
| IFNAR1 | | | | | | | | | |
| IFNA2)- | 0.01463511 | −0.0444027 | −0.1949427 | −0.1114479 | 0.39517644 | −0.0392661 | 0.17500742 | −0.1736553 | −0.2495635 |
| IFNAR1 | | | | | | | | | |
| IL24)- | −0.184286 | 0.17459481 | −0.0018453 | 0.08696225 | −0.0072158 | −0.1663537 | 0.20506063 | 0.05415198 | 0.03860343 |
| IL22RA1 | | | | | | | | | |
| TNF)- | −0.0196473 | −0.0612494 | 0.13108271 | 0.19098459 | 0.02975779 | −0.0666271 | 0.08604559 | 0.11647287 | 0.01508614 |
| CELSR2 | | | | | | | | | |
| PTPN11)- | −0.0189102 | 0.05653105 | 0.07591934 | 0.14547851 | −0.1440952 | 0.04338802 | 0.09666261 | 0.11759281 | 0.05546772 |
| FCRL4 | | | | | | | | | |
| PTPN6)- | 0.09732152 | 0.11003492 | 0.06742017 | −0.2495471 | 0.16453611 | −0.3650438 | 0.08210332 | −0.2693195 | −0.1913106 |
| FCRL4 | | | | | | | | | |
| TFF1)- | −0.0408446 | 0.09512516 | −0.0216783 | −0.0943347 | −0.0136395 | 0.04229388 | −0.0528449 | −0.0974999 | 0.10843215 |
| FCRL4 | | | | | | | | | |
| CXCL12)- | 0.11372255 | −0.1143818 | −0.2142268 | 0.29633046 | 0.23582918 | 0.01785361 | 0.24326108 | 0.12951252 | 0.37549407 |
| ACKR3 | | | | | | | | | |
| CXCL11)- | 0.26124496 | 0.2106963 | −0.124353 | −0.0903251 | 0.34070463 | −0.4005007 | −0.1896131 | 0.25638999 | 0.09130435 |
| ACKR3 | | | | | | | | | |
| IL31)- | 0.05239052 | −0.1436431 | 0.24215868 | −0.0185771 | −0.1318225 | 0.06667545 | −0.1189763 | −0.1001449 | −0.0797101 |
| OSMR | | | | | | | | | |
| S100A4)- | −0.2757848 | 0.29217999 | −0.3920981 | −0.3512516 | 0.10952586 | 0.26542376 | 0.36396589 | 0.15191541 | −0.313834 |
| EGFR | | | | | | | | | |
| NRG1)- | −0.2168739 | −0.1319399 | 0.08706838 | −0.0686475 | 0.0767786 | −0.0655203 | 0.03380672 | −0.0575118 | −0.2208169 |
| EGFR | | | | | | | | | |
| AREG)- | 0.09741948 | 0.09510626 | 0.03736965 | 0.32121212 | 0.06537283 | 0.21403867 | 0.13806512 | −0.0968443 | −0.3065318 |
| EGFR | | | | | | | | | |
| NRG4)- | 0.22775969 | 0.11601848 | 0.09443108 | −0.0066539 | 0.14927341 | −0.0998122 | 0.17430559 | 0.20409104 | −0.3772069 |
| EGFR | | | | | | | | | |
| ANXA1)- | 0.24328865 | −0.1977602 | 0.29266205 | −0.1760211 | 0.31085046 | 0.23617379 | −0.3736532 | −0.2462532 | 0.14532279 |
| EGFR | | | | | | | | | |
| L1CAM)- | −0.161713 | 0.27710001 | −0.0429991 | −0.00975 | −0.01753 | −0.0106726 | 0.03414072 | 0.09316422 | −0.1707735 |
| EGFR | | | | | | | | | |
| EFEMP1)- | 0.0580388 | 0.03596838 | −0.149 | 0.14809935 | 0.10768775 | 0.00787298 | −0.2530561 | −0.0256925 | 0.03741765 |
| EGFR | | | | | | | | | |
| FGL1)- | 0.12007382 | −0.1422782 | 0.29652581 | −0.0892022 | −0.1791281 | −0.0841322 | −0.0707766 | −0.0930202 | −0.1073781 |
| EGFR | | | | | | | | | |
| TGFA)- | −0.0354846 | −0.0108165 | −0.1180352 | −0.0550725 | 0.02715081 | 0.099081 | −0.0394082 | −0.0828091 | −0.0752306 |
| EGFR | | | | | | | | | |
| ARF4)- | 0.07246616 | −0.1570487 | 0.02636001 | −0.2011858 | 0.07321494 | −0.3181923 | −0.0734785 | 0.29678184 | 0.09341238 |
| EGFR | | | | | | | | | |
| GNAI2)- | −0.3042203 | 0.02003031 | −0.1577959 | −0.2346509 | 0.22854132 | 0.1334036 | 0.14768198 | 0.04848645 | −0.1772069 |
| EGFR | | | | | | | | | |
| ANG)- | 0.16379398 | −0.0978118 | 0.21540896 | 0.01238512 | 0.21437987 | 0.08294354 | 0.17931399 | 0.15277183 | 0.09262187 |
| EGFR | | | | | | | | | |
| GSTP1)- | 0.14565697 | −0.4086957 | 0.07150154 | −0.384058 | 0.01047811 | 0.16733094 | −0.1210584 | −0.0992786 | 0.5027668 |
| EGFR | | | | | | | | | |
| HSP90AA1)- | 0.12424652 | −0.4061924 | 0.26162313 | −0.3648221 | 0.22036971 | 0.11950328 | −0.2525948 | −0.1042195 | 0.44756258 |
| EGFR | | | | | | | | | |
| CNTF)- | −0.1259575 | 0.16285476 | 0.12302732 | −0.122406 | 0.07183577 | −0.1141417 | 0.37668459 | 0.42570572 | −0.4073781 |
| EGFR | | | | | | | | | |
| BTC)- | 0.33280613 | −0.164157 | 0.50995451 | −0.2179914 | 0.08853961 | −0.1156203 | 0.15051568 | −0.0799763 | −0.1407115 |
| EGFR | | | | | | | | | |
| CCN2)- | 0.21028361 | −0.127668 | −0.1513065 | −0.1982872 | 0.28613795 | −0.0846536 | −0.1228377 | 0.26041701 | −0.1890646 |
| EGFR | | | | | | | | | |
| LRIG2)- | −0.1982725 | 0.03942902 | −0.027208 | −0.3333443 | 0.13615395 | 0.20693063 | 0.1897262 | 0.08886986 | −0.1126482 |
| EGFR | | | | | | | | | |
| EPGN)- | −0.2829368 | 0.1911227 | −0.0499077 | 0.11107085 | 0.00276789 | −0.045194 | 0.22940557 | 0.29689044 | −0.4029118 |
| EGFR | | | | | | | | | |
| VEGFA)- | 0.07641885 | 0.06969697 | 0.02187881 | 0.06600791 | 0.07506014 | 0.24473797 | 0.22966918 | −0.2414191 | −0.1237195 |
| EGFR | | | | | | | | | |
| CALM1)- | 0.07253442 | 0.0440726 | −0.241927 | −0.0757576 | 0.02306501 | −0.0229915 | −0.0341362 | 0.01258276 | −0.0467721 |
| EGFR | | | | | | | | | |
| SPINK1)- | −0.0919661 | −0.2370224 | −0.0943029 | −0.185639 | −0.1343702 | −0.0145591 | 0.13621537 | 0.1768833 | 0.1972332 |
| EGFR | | | | | | | | | |
| CALM3)- | −0.115752 | −0.0258902 | −0.1044879 | 0.32279315 | −0.020429 | 0.01436147 | 0.04349402 | −0.1156823 | −0.3341238 |
| EGFR | | | | | | | | | |
| EFNA3)- | 0.0650519 | 0.36540364 | 0.00962236 | 0.069304 | −0.2515566 | −0.2144693 | 0.08611148 | −0.0516554 | −0.154809 |
| EPHA1 | | | | | | | | | |
| EFNA1)- | −0.0108058 | 0.13801509 | −0.1574041 | 0.16837945 | −0.2162999 | 0.05541097 | 0.0060614 | −0.0890792 | −0.2826087 |
| EPHA1 | | | | | | | | | |
| EFNA4)- | −0.1443917 | 0.20755774 | 0.09799334 | −0.0018448 | −0.1795598 | −0.1072123 | 0.00230597 | 0.05850766 | −0.0944664 |
| EPHA1 | | | | | | | | | |
| LIN7C)- | −0.0978085 | 0.0590987 | −0.0314003 | 0.34691525 | −0.0395348 | 0.18663504 | 0.2276471 | −0.0265577 | 0.04953887 |
| HTR2C | | | | | | | | | |
| CALM1)- | 0.06834059 | 0.01304391 | 0.26053438 | −0.3023715 | −0.0639784 | −0.0546311 | 0.23594914 | 0.06807474 | −0.0007905 |
| HTR2C | | | | | | | | | |
| FASLG)- | 0.11184189 | −0.04948 | −0.0528195 | 0.00421607 | −0.2170103 | −0.2277997 | −0.1095593 | −0.1488801 | −0.1793149 |
| TNFRSF10B | | | | | | | | | |
| NTF4)- | 0.30353091 | −0.2329807 | −0.2510571 | 0.08558158 | −0.0354786 | −0.0410449 | −0.0821205 | −0.2004349 | −0.016077 |
| NTRK2 | | | | | | | | | |
| BDNF)- | −0.0772729 | 0.28042312 | 0.04559266 | −0.1481896 | 0.14166908 | −0.0226666 | 0.02541091 | 0.05125334 | 0.08063507 |
| NTRK2 | | | | | | | | | |
| DLK1)- | −0.0013838 | −0.0001977 | −0.0089974 | 0.09440364 | −0.0480098 | 0.24118848 | 0.10229033 | −0.1533849 | −0.0077744 |
| NOTCH1 | | | | | | | | | |
| DLL1)- | −0.2049736 | −0.107117 | 0.36280573 | 0.10626173 | −0.1677211 | 0.00658805 | 0.12633037 | −0.2368326 | 0.21607378 |
| NOTCH1 | | | | | | | | | |
| MFNG)- | −0.0304249 | 0.05656274 | 0.08213803 | 0.14321476 | −0.1084715 | 0.02971112 | −0.1004976 | 0.07154386 | 0.11923584 |
| NOTCH1 | | | | | | | | | |
| PSEN1)- | −0.2241112 | −0.1838429 | 0.35344913 | 0.2314458 | −0.1361959 | 0.034326 | −0.1699947 | 0.23170169 | −0.0791857 |
| NOTCH1 | | | | | | | | | |
| TNF)- | 0.01417505 | 0.09177218 | 0.14483101 | −0.0152229 | −0.2932296 | 0.2332938 | −0.1723286 | 0.01198985 | 0.27101449 |
| NOTCH1 | | | | | | | | | |
| WNT4)- | −0.1801167 | −0.1281139 | 0.04690642 | −0.0309649 | 0.19600607 | 0.00098827 | −0.0173976 | 0.11074146 | 0.0027668 |
| NOTCH1 | | | | | | | | | |
| DLK2)- | −0.0721534 | 0.0936975 | −0.0157208 | 0.06192766 | 0.07407652 | −0.0861745 | 0.01581653 | −0.0344226 | 0.18215356 |
| NOTCH1 | | | | | | | | | |
| ADAM17)- | −0.1983651 | −0.1268994 | 0.23453576 | 0.07444005 | 0.21681111 | 0.10547119 | −0.0194799 | −0.0837919 | 0.0185832 |
| NOTCH1 | | | | | | | | | |
| MFAP5)- | −0.2270167 | −0.2537549 | 0.27401234 | 0.00428209 | −0.0216159 | −0.0044799 | 0.05535603 | −0.1482262 | −0.1325428 |
| NOTCH1 | | | | | | | | | |
| MFAP2)- | 0.0760336 | −0.0519172 | 0.14018784 | 0.18584275 | −0.1319033 | 0.21055405 | 0.04402122 | −0.0206858 | −0.0333333 |
| NOTCH1 | | | | | | | | | |
| RBP3)- | 0.01924662 | −0.0330873 | 0.16493588 | 0.02648221 | 0.27421004 | −0.1842617 | −0.0396718 | 0.03867058 | 0.22582345 |
| NOTCH1 | | | | | | | | | |
| JAG2)- | −0.040058 | −0.3294792 | 0.02003559 | 0.04552342 | −0.1952417 | 0.40333366 | −0.1610597 | 0.0881452 | 0.1886693 |
| NOTCH1 | | | | | | | | | |
| PLAU)- | 0.27305374 | −0.1446974 | 0.33070867 | 0.49527323 | −0.5138509 | −0.3559406 | 0.27773386 | 0.37272727 | −0.1496706 |
| ST14 | | | | | | | | | |
| SPINT1)- | 0.4740448 | −0.0915679 | 0.46811594 | −0.2517787 | 0.52687747 | 0.51212121 | −0.2567852 | −0.2100132 | 0.35454545 |
| ST14 | | | | | | | | | |
| GNAI2)- | −0.2991467 | 0.10548857 | −0.2013376 | 0.1969697 | 0.26595955 | 0.05830232 | 0.0942091 | −0.0296452 | 0.24953887 |
| ADRA2A | | | | | | | | | |
| AGT)- | −0.1500049 | 0.19196311 | 0.02799921 | 0.12081686 | 0.0158772 | −0.1911789 | −0.117399 | 0.34836457 | −0.2827404 |
| ADRA2A | | | | | | | | | |
| CXCL12)- | −0.00923 | 0.13574187 | 0.20418663 | −0.1034324 | 0.06074582 | 0.4130843 | 0.05929244 | −0.1092263 | 0.05546772 |
| ADRA2A | | | | | | | | | |
| CXCL3)- | 0.25934227 | −0.263947 | −0.1269072 | −0.0131752 | 0.00368931 | 0.36292368 | 0.09882074 | −0.4579861 | 0.48050066 |
| ADRA2A | | | | | | | | | |
| CCL5)- | −0.1221626 | 0.21657772 | −0.1218042 | −0.1183136 | 0.25370578 | 0.08597121 | 0.23960735 | 0.02233275 | −0.0890646 |
| ADRA2A | | | | | | | | | |
| CXCL11)- | −0.1144797 | −0.1128283 | −0.1843137 | 0.00316237 | 0.11563169 | 0.27441033 | 0.22939588 | 0.07213676 | 0.18208169 |
| ADRA2A | | | | | | | | | |
| NMS)- | −0.0818548 | 0.01787423 | −0.1602191 | −0.1071146 | 0.01903946 | 0.1868968 | 0.16213189 | 0.3662176 | −0.1955204 |
| ADRA2A | | | | | | | | | |
| APLN)- | −0.1622121 | 0.01898234 | −0.0360556 | −0.1475723 | 0.13420741 | 0.1360477 | −0.0564596 | 0.06284792 | 0.04888011 |
| ADRA2A | | | | | | | | | |
| SST)- | −0.2458568 | 0.13837639 | 0.02286656 | −0.2429673 | 0.08696798 | 0.24412821 | 0.41050135 | 0.03366382 | 0.01633729 |
| ADRA2A | | | | | | | | | |
| NPY)- | 0.43338718 | −0.2767234 | 0.20213538 | −0.0930891 | −0.0672684 | 0.28892842 | 0.44963436 | 0.26344741 | −0.1555995 |
| ADRA2A | | | | | | | | | |
| POMC)- | 0.04916629 | 0.1293703 | −0.223826 | −0.0859854 | 0.16061545 | 0.09686028 | −0.1882927 | −0.069438 | −0.1009915 |
| ADRA2A | | | | | | | | | |
| CXCL8)- | −0.0497414 | 0.26451018 | 0.1485703 | −0.0187088 | −0.1695764 | 0.02048816 | −0.0021082 | −0.3370335 | 0.14453228 |
| ADRA2A | | | | | | | | | |
| CXCL2)- | 0.18306814 | −0.2283156 | −0.1944048 | −0.2532279 | −0.2835496 | 0.13946441 | 0.2017986 | −0.3958759 | 0.41424289 |
| ADRA2A | | | | | | | | | |
| CXCL13)- | −0.0009887 | −0.0586588 | −0.0794252 | 0.05777529 | 0.02727542 | −0.0766849 | 0.26497134 | −0.0426233 | −0.0214756 |
| ADRA2A | | | | | | | | | |
| CXCL10)- | −0.1851437 | 0.1435492 | 0.05747619 | 0.03715537 | 0.01344006 | 0.04908097 | 0.26727716 | 0.18564511 | 0.00988142 |
| ADRA2A | | | | | | | | | |
| PPBP)- | −0.1860687 | 0.15393741 | −0.0962831 | −0.0740472 | 0.17472082 | 0.02694512 | 0.101983 | 0.19249646 | −0.0581028 |
| ADRA2A | | | | | | | | | |
| CXCL5)- | 0.02786745 | −0.1019796 | 0.07793919 | −0.2472991 | 0.06482641 | 0.12931915 | 0.21878912 | −0.1444712 | 0.30658762 |
| ADRA2A | | | | | | | | | |
| CCL19)- | −0.0660231 | 0.14923898 | 0.10885609 | 0.00408459 | 0.19126367 | 0.04134137 | 0.08571052 | 0.14032083 | −0.0094862 |
| ADRA2A | | | | | | | | | |
| APP)- | −0.0392648 | 0.07286142 | 0.2428435 | 0.19025033 | −0.0250346 | −0.286966 | 0.00711509 | 0.27122105 | −0.357971 |
| ADRA2A | | | | | | | | | |
| CXCL1)- | −0.167611 | −0.0290543 | −0.1766431 | 0.01554677 | 0.18334541 | −0.0279983 | 0.41128023 | 0.13730852 | 0.00026354 |
| ADRA2A | | | | | | | | | |
| NPB)- | −0.2112546 | 0.30072811 | −0.1953478 | −0.2111989 | 0.2036366 | 0.45548272 | 0.34791488 | 0.05533779 | −0.0418972 |
| ADRA2A | | | | | | | | | |
| PPY)- | −0.0928746 | 0.13597867 | −0.1974556 | −0.3287328 | 0.02931779 | 0.41685882 | 0.44008169 | 0.34118383 | −0.0166008 |
| ADRA2A | | | | | | | | | |
| CXCL9)- | −0.0984417 | −0.0119918 | −0.1298102 | −0.2107517 | 0.25253657 | 0.29719669 | 0.25423282 | 0.23531737 | −0.1255599 |
| ADRA2A | | | | | | | | | |
| CXCL16)- | 0.09612597 | −0.0414402 | −0.248394 | −0.0101449 | 0.08676461 | 0.1868968 | 0.03399433 | −0.1766857 | 0.02108037 |
| ADRA2A | | | | | | | | | |
| CCL20)- | 0.17166393 | −0.1810155 | −0.0834953 | −0.2885375 | 0.08347058 | 0.25145756 | 0.14448068 | −0.0425588 | 0.25560789 |
| ADRA2A | | | | | | | | | |
| NPW)- | 0.09101394 | −0.1674575 | −0.1493113 | −0.0193676 | −0.1397984 | 0.16324648 | 0.1475723 | 0.24631905 | −0.0641634 |
| ADRA2A | | | | | | | | | |
| PENK)- | 0.15242117 | −0.1570968 | −0.0124934 | −0.223269 | 0.11197127 | 0.44852917 | 0.07859543 | 0.24598966 | −0.0743083 |
| ADRA2A | | | | | | | | | |
| CCL28)- | −0.0241781 | 0.09993742 | 0.34746516 | 0.07720685 | 0.12655643 | −0.0017787 | 0.19579682 | 0.12022794 | −0.0003953 |
| ADRA2A | | | | | | | | | |
| PF4)- | −0.0451247 | 0.07124723 | 0.03813297 | −0.0807695 | 0.00092239 | 0.17910202 | −0.0356755 | 0.27913565 | −0.0528344 |
| ADRA2A | | | | | | | | | |
| ANXA1)- | −0.4879607 | 0.49841897 | 0.03946242 | 0.52345191 | 0.04025298 | −0.4721499 | −0.0687134 | 0.45258408 | −0.4631094 |
| ADRA2A | | | | | | | | | |
| EFNA3)- | 0.14581411 | 0.01831961 | 0.03898758 | −0.1297144 | 0.04283079 | −0.0941382 | 0.01528679 | −0.2644481 | 0.05955204 |
| EPHA4 | | | | | | | | | |
| EFNA1)- | −0.0798708 | −0.3117362 | 0.36930022 | −0.2873518 | 0.2893289 | −0.1103789 | −0.28867 | −0.0738715 | 0.24782609 |
| EPHA4 | | | | | | | | | |
| EFNA4)- | 0.21514023 | 0.13422067 | 0.01113821 | −0.0968507 | 0.12797786 | 0.02590127 | −0.1560966 | 0.00336079 | 0.16666667 |
| EPHA4 | | | | | | | | | |
| PDGFC)- | 0.2723679 | 0.22279315 | −0.2563934 | 0.03346619 | 0.02399236 | −0.3186296 | −0.2697074 | 0.23455001 | 0.25797101 |
| FLT1 | | | | | | | | | |
| VEGFC)- | −0.2398102 | −0.2242283 | 0.17450639 | 0.16924141 | −0.1719013 | 0.065887 | −0.4592012 | 0.37910133 | −0.0022398 |
| FLT1 | | | | | | | | | |
| PGF)- FLT1 | 0.03604375 | −0.1761102 | 0.07050099 | −0.1202899 | 0.10031639 | −0.1442878 | −0.2089375 | 0.28712611 | 0.18603426 |
| VEGFB)- | 0.41251853 | 0.04723476 | −0.3042877 | 0.17142104 | −0.1797509 | −0.1185346 | −0.2420907 | 0.35208855 | 0.05270092 |
| FLT1 | | | | | | | | | |
| VEGFA)- | −0.0367637 | 0.34057971 | 0.13399685 | 0.4002635 | 0.43949387 | −0.391883 | −0.1686056 | 0.13440949 | −0.3160183 |
| FLT1 | | | | | | | | | |
| SORBS1)- | −0.2555351 | 0.09316729 | 0.01097235 | −0.268226 | −0.067743 | 0.29835601 | 0.07445477 | −0.4238891 | 0.23123291 |
| ITGA1 | | | | | | | | | |
| COL4A5)- | −0.064024 | 0.14675454 | 0.12235953 | −0.028195 | −0.1048262 | −0.0984913 | 0.07000725 | −0.1345653 | 0.10349485 |
| ITGA1 | | | | | | | | | |
| LAMA2)- | 0.09134932 | 0.27043942 | 0.37623958 | −0.0048748 | 0.00474386 | −0.0713486 | 0.29846813 | 0.0982937 | 0.24769433 |
| ITGA1 | | | | | | | | | |
| LAMA1)- | 0.04705107 | 0.19741698 | 0.01631133 | 0.10219411 | 0.15012027 | −0.2527675 | 0.02319223 | 0.29138942 | 0.07233202 |
| ITGA1 | | | | | | | | | |
| COL4A4)- | −0.0823495 | −0.2624985 | −0.2889609 | 0.06045026 | 0.03988924 | 0.02063487 | 0.18593925 | −0.178542 | 0.28722949 |
| ITGA1 | | | | | | | | | |
| COL5A1)- | −0.2680018 | 0.3256917 | 0.09369132 | 0.04110807 | 0.1424524 | −0.0022071 | 0.19680448 | −0.3444232 | 0.3801054 |
| ITGA1 | | | | | | | | | |
| COL6A2)- | −0.2305158 | 0.1599473 | 0.18468127 | −0.0175231 | −0.0175259 | 0.03550959 | 0.20477681 | −0.2769616 | 0.24848485 |
| ITGA1 | | | | | | | | | |
| COL4A3)- | −0.3069164 | −0.057885 | −0.1229106 | −0.0542927 | −0.0401331 | 0.08427781 | −0.1053533 | −0.0575137 | −0.0990777 |
| ITGA1 | | | | | | | | | |
| COL5A2)- | −0.2344028 | 0.3055336 | 0.13045627 | 0.01370224 | 0.11345742 | 0.07141445 | 0.2144622 | −0.2578562 | 0.37338603 |
| ITGA1 | | | | | | | | | |
| COL1A2)- | −0.2107517 | 0.28735178 | 0.16755065 | 0.06455863 | 0.17736782 | 0.02483695 | 0.29451491 | −0.2571974 | 0.36732543 |
| ITGA1 | | | | | | | | | |
| MATN1)- | −0.059248 | −0.2131207 | −0.078401 | 0.09275973 | −0.0676045 | −0.1988404 | 0.03281173 | −0.1274129 | 0.25349144 |
| ITGA1 | | | | | | | | | |
| PLA2G10)- | 0.12685756 | 0.13671553 | 0.24301529 | 0.02312329 | −0.0699038 | 0.10845716 | −0.1713608 | 0.12308098 | −0.1492754 |
| PLA2R1 | | | | | | | | | |
| FN1)- | 0.41963109 | −0.1891963 | 0.33939394 | 0.60447958 | −0.4263505 | −0.4823452 | 0.35849802 | 0.42859025 | −0.1086957 |
| ITGB6 | | | | | | | | | |
| FYN)- | 0.28139413 | −0.1690605 | −0.1780867 | 0.22714097 | 0.18076416 | −0.2054018 | 0.29459816 | −0.498419 | 0.39710145 |
| THY1 | | | | | | | | | |
| BGN)- | −0.1494071 | 0.15375494 | 0.07103524 | −0.2599473 | 0.01332323 | −0.2632411 | 0.09346048 | −0.1391304 | 0.13517787 |
| LY96 | | | | | | | | | |
| WNT11)- | 0.13460715 | −0.3074946 | −0.0004613 | −0.1637789 | −0.1507495 | −0.073325 | 0.06140467 | −0.0034257 | 0.25949471 |
| FZD7 | | | | | | | | | |
| CSF1)- | −0.0965744 | 0.07075099 | −0.1865474 | 0.09097796 | 0.03855175 | 0.21687144 | −0.1221865 | 0.08893281 | 0.1826087 |
| CSF1R | | | | | | | | | |
| CSF2)- | −0.1257045 | −0.1198379 | −0.0451138 | −0.1834832 | 0.11414446 | −0.2971967 | −0.2212032 | 0.01277997 | 0.05335968 |
| CSF1R | | | | | | | | | |
| WNT4)- | 0.16631503 | 0.07038356 | −0.1224577 | −0.2966696 | −0.1235418 | −0.0772172 | −0.0803321 | 0.15369408 | 0.04756258 |
| FZD1 | | | | | | | | | |
| WNT7B)- | −0.2488959 | 0.16049546 | 0.09527105 | 0.29104084 | 0.21279121 | −0.3946484 | −0.1661999 | 0.03638042 | 0.04202899 |
| FZD1 | | | | | | | | | |
| B2M)- | −0.1438925 | 0.11081099 | 0.02385267 | 0.21884058 | 0.03149399 | 0.11054747 | 0.08005271 | 0.1847289 | 0.21594203 |
| CD1A | | | | | | | | | |
| IFNL1)- | −0.2266495 | −0.1025666 | 0.08175168 | 0.01963174 | 0.06740019 | −0.0654366 | 0.09783898 | −0.0841516 | −0.3312362 |
| IL10RB | | | | | | | | | |
| IL10)- | −0.2696414 | −0.1535268 | −0.0228666 | 0.2081439 | −0.1603242 | 0.28716428 | 0.15245751 | −0.0781549 | −0.1587668 |
| IL10RB | | | | | | | | | |
| IL24)- | −0.0957622 | −0.1029155 | 0.21988666 | −0.0507297 | 0.20461883 | 0.03621326 | −0.0975754 | −0.1459638 | −0.26852 |
| IL10RB | | | | | | | | | |
| UCN3)- | −0.0638782 | −0.094998 | −3.30E−05 | −0.2144622 | −0.1786373 | 0.24372241 | −0.0665129 | −0.1095918 | −0.0698333 |
| IL10RB | | | | | | | | | |
| PENK)- | −0.1361179 | 0.10502376 | 0.15908192 | 0.09915667 | −0.351371 | −0.0549516 | 0.08376723 | 0.06832257 | 0.18130312 |
| OGFR | | | | | | | | | |
| GPI)- | −0.0946013 | −0.0379459 | −0.0129211 | −0.1396574 | −0.0543195 | −0.0824769 | 0.01404134 | 0.09288538 | 0.12371542 |
| AMFR | | | | | | | | | |
| TNFSF13B)- | −0.0642897 | 0.03842099 | 0.07761293 | 0.04262327 | 0.13585579 | −0.0575796 | −0.1834213 | 0.06456501 | 0.14579353 |
| TNFRSF13C | | | | | | | | | |
| GHRL)- | 0.01252348 | −0.0827868 | −0.0525518 | −0.0328754 | −0.092401 | 0.04809434 | 0.00514037 | −0.1500708 | 0.04967226 |
| PTGIR | | | | | | | | | |
| GNAS)- | −0.0899239 | −0.1704931 | −0.083564 | −0.1540337 | −0.1908324 | 0.03070132 | 0.14913669 | −0.053625 | 0.28116868 |
| PTGIR | | | | | | | | | |
| GNAI2)- | −0.4095012 | −0.4954207 | 0.50079105 | 0.04255599 | 0.0412575 | −0.0951252 | 0.23996575 | −0.1952569 | −0.1437418 |
| CCR5 | | | | | | | | | |
| S100A4)- | −0.0090915 | −0.0695039 | −0.0506855 | −0.0255599 | 0.08534898 | −0.2927536 | 0.25538789 | −0.0891963 | −0.0125165 |
| CCR5 | | | | | | | | | |
| CCL5)- | −0.3913158 | −0.4529222 | 0.4805867 | 0.38115942 | −0.3416596 | 0.28458498 | −0.3693403 | 0.35270092 | 0.42964427 |
| CCR5 | | | | | | | | | |
| CCL14)- | −0.0546167 | −0.0649603 | 0.35622716 | 0.02747126 | −0.2330532 | 0.00210811 | 0.08126278 | −0.1843215 | −0.3465086 |
| CCR5 | | | | | | | | | |
| CCL11)- | 0.04687038 | 0.16546363 | −0.1480956 | −0.0480253 | 0.23911685 | −0.0274054 | −0.0678837 | 0.22542819 | 0.14980237 |
| CCR5 | | | | | | | | | |
| CCL7)- | −0.3012436 | 0.21562631 | 0.05863334 | 0.13458941 | −0.115538 | 0.446128 | −0.1397878 | 0.29868248 | 0.23649539 |
| CCR5 | | | | | | | | | |
| CCL3L3)- | 0.11988007 | 0.07025407 | −0.0969241 | −0.1417655 | 0.16239374 | −0.1349144 | 0.09345549 | 0.00948617 | 0.08076416 |
| CCR5 | | | | | | | | | |
| CCL8)- | −0.0178548 | 0.1698511 | −0.0727375 | 0.04697279 | 0.0114685 | 0.03228144 | 0.00474527 | 0.24664032 | −0.0724638 |
| CCR5 | | | | | | | | | |
| MDK)- | −0.1046148 | 0.12898551 | −0.0302581 | −0.0295125 | −0.1183296 | 0.08695939 | −0.2977784 | 0.11977074 | −0.0696334 |
| GPC2 | | | | | | | | | |
| FGF19)- | −0.0198333 | −0.1877183 | 0.22353371 | 0.12924901 | 0.09737287 | −0.0433479 | 0.06342092 | 0.16206067 | −0.1616601 |
| FGFR4 | | | | | | | | | |
| FGF21)- | −0.2046601 | −0.1672159 | 0.10042545 | −0.2057971 | 0.06065203 | 0.22517211 | 0.02907437 | 0.06904276 | 0.00052703 |
| FGFR4 | | | | | | | | | |
| FGF14)- | −0.0190671 | −0.3602841 | 0.14010303 | 0.08448384 | 0.17480051 | −0.1818901 | 0.08135283 | 0.06693238 | −0.1413702 |
| FGFR4 | | | | | | | | | |
| FGF11)- | 0.37647062 | −0.2041785 | −0.0822478 | −0.4911726 | 0.32119204 | −0.0496723 | 0.05491645 | 0.30442373 | −0.2766798 |
| FGFR4 | | | | | | | | | |
| FGF17)- | 0.02807526 | 0.01772769 | 0.26328114 | −0.1740505 | −0.0244264 | 0.24972001 | −0.0859017 | 0.13294246 | 0.00158103 |
| FGFR4 | | | | | | | | | |
| FGF12)- | −0.3981415 | 0.11671665 | −0.0118388 | 0.02878978 | −0.0534037 | 0.20459861 | 0.02663415 | 0.03386146 | −0.2990777 |
| FGFR4 | | | | | | | | | |
| L1CAM)- | −0.0628444 | −0.1452136 | −0.0117771 | 0.07589183 | 0.1036959 | −0.0801766 | −0.0146928 | 0.27619832 | −0.0777441 |
| ITGA5 | | | | | | | | | |
| PLAU)- | −0.2915129 | 0.22468949 | 0.26637454 | −0.232287 | −0.2649713 | 0.68166546 | 0.21594914 | −0.0121875 | 0.23939394 |
| ITGA5 | | | | | | | | | |
| SPP1)- | 0.27136175 | −0.2853849 | −0.1651624 | 0.16007905 | 0.07688 | −0.0787246 | 0.23228697 | −0.2043546 | 0.30988142 |
| ITGA5 | | | | | | | | | |
| CCN1)- | −0.3374288 | 0.44189723 | 0.5859218 | −0.4467721 | −0.4031095 | 0.42135775 | 0.66978491 | −0.4587108 | 0.57891963 |
| ITGA5 | | | | | | | | | |
| FGG)- | −0.0471422 | 0.25866386 | 0.24582441 | 0.00994795 | 0.01324242 | −0.1765326 | 0.0698992 | 0.09368206 | 0.03761652 |
| ITGA5 | | | | | | | | | |
| FGA)- | 0.07077141 | −0.0102889 | −0.1092571 | −0.0184459 | 0.02806509 | −0.1174979 | 0.05079219 | −0.0729273 | 0.27180501 |
| ITGA5 | | | | | | | | | |
| CCN2)- | −0.1783326 | 0.3516469 | 0.4118054 | −0.1781291 | −0.3410521 | 0.12477354 | 0.51279686 | −0.2072532 | 0.46982872 |
| ITGA5 | | | | | | | | | |
| FGB)- | −0.1498715 | 0.07249482 | −0.0278455 | 0.05792231 | −0.027084 | −0.0348929 | −0.1502784 | 0.02371776 | −0.1013242 |
| ITGA5 | | | | | | | | | |
| ANGPT1)- | −0.1146439 | 0.20091702 | 0.19442627 | −0.0076421 | −0.0424943 | −0.1587113 | 0.2656214 | −0.1578445 | 0.34545455 |
| ITGA5 | | | | | | | | | |
| COL18A1)- | −0.0541716 | 0.22438962 | 0.2332279 | −0.2327481 | −0.2555834 | 0.4305949 | 0.23999473 | −0.0073784 | 0.21449275 |
| ITGA5 | | | | | | | | | |
| ADAM15)- | 0.11443065 | 0.12042161 | 0.09038506 | −0.0561265 | −0.1353141 | 0.23090352 | 0.24684608 | −0.1058006 | 0.21343874 |
| ITGA5 | | | | | | | | | |
| ADAM17)- | −0.1129936 | 0.09314746 | 0.00224141 | −0.0885375 | 0.099081 | 0.42175302 | −0.0738739 | 0.23625162 | 0.04276771 |
| ITGA5 | | | | | | | | | |
| FN1)- | −0.4123324 | 0.54888011 | 0.49916005 | −0.5517787 | −0.4770249 | 0.86280839 | 0.44118713 | −0.2690471 | 0.5312253 |
| ITGA5 | | | | | | | | | |
| GNAS)- | 0.23913043 | 0.03873518 | −0.1102908 | −0.053495 | −0.13423 | 0.20146255 | −0.1895973 | −0.1176548 | 0.03188406 |
| ADCY9 | | | | | | | | | |
| GNAI2)- | 0.05033933 | 0.03966528 | −0.0735857 | −0.141502 | −0.0171402 | 0.05019763 | −0.0090975 | −0.102108 | −0.2794466 |
| ADCY9 | | | | | | | | | |
| WNT8A)- | 0.4176213 | −0.3214289 | 0.24410347 | 0.06383609 | −0.0291173 | 0.08762683 | 0.00500659 | 0.03557781 | −0.0718074 |
| LRP5 | | | | | | | | | |
| SOST)- | −0.0049554 | 0.01070265 | 0.13285381 | −0.0837341 | −0.1320202 | −0.1879097 | 0.07786561 | 0.19146133 | −0.1849205 |
| LRP5 | | | | | | | | | |
| WNT7B)- | −0.0152214 | 0.14089936 | 0.04091448 | 0.05797292 | 0.17720685 | 0.19567796 | −0.0338603 | 0.22427197 | −0.3335419 |
| LRP5 | | | | | | | | | |
| DKK1)- | 0.13568369 | −0.2419201 | 0.06101338 | 0.07306147 | −0.0820185 | 0.10548509 | −0.0972332 | −0.2544472 | 0.19756909 |
| LRP5 | | | | | | | | | |
| PSEN1)- | 0.09989457 | −0.3430417 | 0.17225701 | 0.11298877 | 0.06450338 | 0.0266166 | −0.1351912 | −0.0067855 | 0.20527685 |
| NCSTN | | | | | | | | | |
| GNAI2)- | −0.1208567 | 0.14284773 | −0.1197388 | −0.0129117 | 0.11555975 | −0.1488997 | 0.14656037 | −0.2427197 | −0.302635 |
| LHCGR | | | | | | | | | |
| GNAS)- | 0.29042035 | −0.1524374 | −0.0220962 | 0.03070031 | −0.0406003 | −0.1443632 | −0.0475563 | 0.20147582 | −0.0561265 |
| LHCGR | | | | | | | | | |
| GAS6)- | −0.0367613 | 0.10790514 | −0.0334794 | −0.2927536 | 0.0828418 | −0.1678635 | −0.0140376 | −0.0143619 | 0.16205534 |
| MERTK | | | | | | | | | |
| PROS1)- | 0.03139516 | −0.0102118 | −0.0278144 | −0.1501976 | 0.08468713 | −0.1005995 | 0.17253766 | −0.2006061 | −0.3123847 |
| MERTK | | | | | | | | | |
| CCK)- | −0.2180117 | −0.1028767 | −0.0326453 | 0.0887381 | 0.15353685 | −0.1814948 | −0.0003955 | 0.17279884 | 0.15336474 |
| CCKAR | | | | | | | | | |
| YARS1)- | −0.1589718 | −0.0865869 | 0.29054835 | −0.0824769 | −0.3942412 | 0.15701391 | 0.2529406 | −0.0083679 | 0.17799736 |
| CXCR1 | | | | | | | | | |
| GNAI2)- | 0.02853565 | −0.0735982 | −0.096036 | −0.0259552 | 0.24739162 | −0.0512618 | 0.16990421 | 0.07359821 | −0.0083004 |
| CXCR1 | | | | | | | | | |
| CXCL3)- | 0.09933753 | 0.07658089 | −0.259856 | 0.12108037 | −0.1235967 | 0.15259933 | −0.0774874 | 0.10931014 | 0.05797101 |
| CXCR1 | | | | | | | | | |
| CXCL8)- | 0.12493411 | 0.06713222 | 0.00469051 | −0.2455863 | 0.08600905 | −0.2635567 | 0.07960131 | 0.02714634 | 0.2571805 |
| CXCR1 | | | | | | | | | |
| CXCL2)- | 0.03290615 | −0.1168664 | 0.1320636 | −0.0509881 | −0.1901183 | −0.0062595 | 0.14619378 | −0.0459922 | 0.05514016 |
| CXCR1 | | | | | | | | | |
| PPBP)- | −0.2399156 | 0.11650742 | 0.23329879 | −0.0406469 | 0.04815873 | 0.34556716 | 0.11639627 | −0.2920208 | 0.03003953 |
| CXCR1 | | | | | | | | | |
| CXCL5)- | −0.0140349 | −0.1301097 | −0.015062 | −0.0843215 | −0.1468496 | −0.0222705 | 0.26060346 | 0.08374514 | 0.11475626 |
| CXCR1 | | | | | | | | | |
| CXCL1)- | −0.0977233 | 0.17682907 | 0.06216783 | 0.08801054 | 0.1237949 | −0.1045002 | 0.16629286 | 0.00395387 | −0.048623 |
| CXCR1 | | | | | | | | | |
| IL17F)- | −0.1655902 | −0.0820898 | 0.03670269 | 0.25774996 | −0.1198023 | 0.10222779 | −0.2551804 | 0.04533922 | 0.13781291 |
| IL17RC | | | | | | | | | |
| FN1)- | 0.23572442 | −0.1664032 | −0.1211662 | 0.28405797 | 0.03669906 | 0.01680451 | −0.1613573 | 0.34274608 | −0.0930171 |
| IL17RC | | | | | | | | | |
| CCL5)- | −0.1342821 | 0.03637082 | 0.21333908 | −0.0625823 | −0.2363373 | 0.16587615 | −0.0671765 | 0.06284585 | −0.084058 |
| CCR1 | | | | | | | | | |
| CCL14)- | −0.1262312 | −0.1400665 | 0.17619914 | −0.0863665 | 0.09143952 | 0.15231068 | 0.15208653 | −0.03083 | 0.01541502 |
| CCR1 | | | | | | | | | |
| CCL15)- | 0.07874515 | 0.12941422 | −0.1226988 | 0.04473138 | 0.01878894 | 0.22207583 | 0.08761292 | −0.1886693 | −0.1501976 |
| CCR1 | | | | | | | | | |
| CCL23)- | 0.08797075 | −0.128892 | 0.08463847 | 0.15046609 | −0.1196559 | 0.1571198 | −0.0002637 | 0.00737813 | −0.0561265 |
| CCR1 | | | | | | | | | |
| CCL7)- | 0.07902121 | 0.07875736 | 0.13607712 | 0.13788333 | −0.2065465 | 0.08590533 | 0.13995653 | −0.0462451 | −0.0388669 |
| CCR1 | | | | | | | | | |
| CCL8)- | 0.02813282 | −0.1687311 | 0.25759878 | 0.05533961 | 0.0648075 | 0.04736807 | 0.10429167 | −0.0067194 | −0.0608696 |
| CCR1 | | | | | | | | | |
| CCL18)- | 0.04863423 | −0.0102145 | 0.09252482 | 0.07312494 | −0.0831988 | −0.1775421 | 0.05412356 | −0.1230567 | −0.0279315 |
| CCR1 | | | | | | | | | |
| PLAU)- | −0.0712309 | −0.085527 | 0.25341812 | −0.1547482 | −0.344346 | 0.31678635 | 0.3944664 | −0.0656148 | 0.29393939 |
| ITGB5 | | | | | | | | | |
| EDIL3)- | −0.3153595 | 0.39802372 | 0.35467721 | −0.3282058 | −0.1481603 | 0.3883655 | 0.18945982 | −0.2463849 | 0.33491436 |
| ITGB5 | | | | | | | | | |
| SORBS1)- | −0.1068758 | 0.15483956 | 0.27831588 | −0.198254 | −0.016933 | 0.2872768 | 0.06363846 | −0.1643059 | 0.16304885 |
| ITGB5 | | | | | | | | | |
| SPP1)- | 0.22346663 | −0.0946013 | −0.0845219 | 0.10105402 | 0.01343874 | −0.0642972 | 0.03280632 | −0.2832109 | 0.15467721 |
| ITGB5 | | | | | | | | | |
| TLN1)- | 0.08162593 | 0.05131921 | 0.14025495 | −0.1025725 | −0.0312922 | 0.34112919 | 0.25032938 | −0.0468395 | 0.23860343 |
| ITGB5 | | | | | | | | | |
| COL4A2)- | −0.3038967 | 0.36218709 | 0.42332016 | −0.1586298 | −0.2486166 | 0.03794591 | 0.45388669 | −0.2068579 | 0.39736495 |
| ITGB5 | | | | | | | | | |
| CCN1)- | −0.3280082 | 0.32766798 | 0.41040843 | −0.2913043 | −0.5014493 | 0.18221944 | 0.54097497 | −0.4256398 | 0.39104084 |
| ITGB5 | | | | | | | | | |
| ADAM9)- | −0.1087651 | 0.22938076 | 0.21910408 | 0.1483531 | 0.15586298 | −0.0781317 | −0.0673254 | 0.15725156 | −0.0096179 |
| ITGB5 | | | | | | | | | |
| COL18A1)- | −0.0647819 | 0.23552672 | 0.17990709 | 0.09262492 | 0.13386475 | 0.25357402 | 0.08418972 | 0.0179189 | 0.16903821 |
| ITGB5 | | | | | | | | | |
| ITGB3BP)- | −0.0667743 | 0.13347837 | 0.05055699 | 0.02846037 | −0.0470387 | 0.17936555 | 0.15428195 | 0.19763497 | 0.11067194 |
| ITGB5 | | | | | | | | | |
| LTBP3)- | −0.1502026 | 0.33175231 | 0.10342556 | −0.1735178 | −0.0393939 | −0.1206232 | 0.29815547 | −0.0521098 | 0.46521739 |
| ITGB5 | | | | | | | | | |
| LTBP1)- | −0.0629138 | 0.3685112 | 0.29459816 | −0.3760211 | −0.2025033 | 0.146184 | 0.24756258 | −0.0630456 | 0.39275362 |
| ITGB5 | | | | | | | | | |
| MST1)- | −0.0287428 | −0.1264421 | −0.040182 | −0.3396574 | 0.19664679 | 0.28353096 | 0.03188511 | 0.0284585 | −0.2032938 |
| MST1R | | | | | | | | | |
| SHH)- | 0.21553059 | 0.03169269 | −0.1355982 | −0.1189723 | 0.0662867 | −0.2365691 | −0.0996277 | 0.14881913 | 0.01396574 |
| HHIP | | | | | | | | | |
| COL4A5)- | 0.00118616 | −0.0839649 | −0.1281713 | −0.2313875 | 0.12028986 | 0.21488801 | −0.0453243 | 0.0857077 | −0.0677318 |
| ITGA2 | | | | | | | | | |
| LAMA2)- | 0.18005139 | −0.1095737 | 0.08175769 | −0.0115957 | 0.00013175 | −0.0534914 | 0.10500659 | 0.27588933 | −0.0610094 |
| ITGA2 | | | | | | | | | |
| LAMA1)- | 0.02563258 | −0.1149334 | 0.04994729 | 0.14991763 | −0.0811755 | −0.158727 | −0.0339921 | 0.01857708 | −0.0990908 |
| ITGA2 | | | | | | | | | |
| LAMC3)- | −0.0212184 | 0.10861041 | 0.02912589 | −0.1247982 | 0.03162368 | 0.07003327 | −0.2523057 | −0.1779974 | −0.0797206 |
| ITGA2 | | | | | | | | | |
| COL4A4)- | 0.00672735 | −0.1416228 | 0.09978902 | 0.21750457 | −0.1048815 | −0.1802301 | −0.2142363 | −0.0326756 | 0.11365508 |
| ITGA2 | | | | | | | | | |
| COL11A1)- | 0.09196311 | −0.0591646 | 0.11238472 | 0.51785479 | −0.3534914 | −0.4300395 | 0.15902503 | 0.23162055 | −0.2258532 |
| ITGA2 | | | | | | | | | |
| COL6A2)- | 0.25230567 | −0.1531164 | 0.34664032 | 0.49400448 | −0.4434783 | −0.4859025 | 0.36982872 | 0.34650856 | −0.2595862 |
| ITGA2 | | | | | | | | | |
| COL4A3)- | −0.0243935 | −0.0339575 | −0.0559072 | −0.0025041 | 0.15800224 | 0.05284312 | 0.24044796 | 0.33544137 | −0.5024377 |
| ITGA2 | | | | | | | | | |
| MMP1)- | −0.4092916 | 0.38456469 | −0.218056 | 0.13308736 | 0.11699605 | −0.0227931 | 0.07285903 | 0.25467721 | −0.1897483 |
| ITGA2 | | | | | | | | | |
| HSPG2)- | 0.05678524 | 0.14534194 | 0.19038208 | 0.06114113 | −0.1096179 | −0.112253 | −0.1418972 | −0.2740448 | 0.39675847 |
| ITGA2 | | | | | | | | | |
| LAMB3)- | 0.44216074 | −0.383845 | 0.4458498 | −0.4830676 | 0.46034256 | 0.49578393 | −0.5418972 | −0.6017128 | 0.67189354 |
| ITGA2 | | | | | | | | | |
| COL1A2)- | 0.15902503 | −0.0740546 | 0.19051383 | 0.54012387 | −0.4397892 | −0.4537549 | 0.06824769 | 0.08471673 | −0.0416392 |
| ITGA2 | | | | | | | | | |
| FN1)- | 0.25349144 | −0.1602319 | 0.24519104 | 0.56239294 | −0.5009223 | −0.4710145 | 0.17654809 | 0.25665349 | −0.2010805 |
| ITGA2 | | | | | | | | | |
| COL7A1)- | −0.0459346 | 0.01227725 | 0.01913942 | 0.18765237 | −0.0900586 | −0.0880822 | 0.1507296 | 0.09196614 | −0.0169988 |
| ITGA2 | | | | | | | | | |
| COL3A1)- | 0.08827404 | −0.0127817 | 0.10395257 | 0.46712347 | −0.4047431 | −0.4147563 | 0.03359684 | 0.06666667 | −0.0084333 |
| ITGA2 | | | | | | | | | |
| F2)- F2RL2 | 0.11099763 | −6.59E−05 | −0.0660732 | 0.15883787 | −0.0362618 | −0.0457885 | 0.17714931 | 0.08445879 | 0.24230047 |
| F2)- F2RL1 | 0.13501137 | 0.02464661 | −0.0200013 | −0.2796995 | 0.14028267 | 0.17794321 | 0.32410885 | 0.35389835 | −0.318959 |
| NUCB2)- | 0.10455923 | 0.26340757 | −0.0218108 | −0.0883457 | −0.0052052 | 0.09783253 | 0.34859666 | 0.1173913 | −0.041502 |
| ERAP1 | | | | | | | | | |
| TNF)- | 0.07067513 | 0.01687764 | −0.0160865 | 0.17226269 | −0.0465254 | −0.1532176 | 0.37140975 | 0.4428195 | −0.4503294 |
| TNFRSF21 | | | | | | | | | |
| TNFSF11)- | 0.1504317 | −0.0316341 | 0.27782906 | 0.1302013 | 0.04609252 | 0.11705983 | −0.2697059 | −0.1407161 | 0.243083 |
| TNFRSF11B | | | | | | | | | |
| FN1)- | 0.18281235 | −0.1293808 | 0.11594585 | 0.42727273 | −0.2188478 | −0.3235943 | 0.15428703 | 0.16449817 | −0.1320158 |
| TNFRSF11B | | | | | | | | | |
| INSL3)- | 0.01417038 | −0.1801885 | 0.01062005 | 0.09288538 | −0.0121316 | 0.22385454 | −0.1897541 | −0.0243091 | 0.30368906 |
| RXFP2 | | | | | | | | | |
| FAT4)- | 0.14740485 | −0.1031961 | −0.0119091 | −0.0743132 | 0.02193201 | 0.05656772 | 0.06187543 | 0.11927119 | −0.0437475 |
| DCHS1 | | | | | | | | | |
| ADM)- | −0.1857209 | −0.1541858 | −0.0379342 | 0.13979841 | −0.383893 | 0.16994433 | −0.0966785 | 0.05771131 | 0.26331566 |
| GPR182 | | | | | | | | | |
| B2M)- | −0.3817775 | −0.4155741 | 0.52159252 | 0.11660079 | −0.0758618 | 0.14110672 | −0.1762186 | 0.16772069 | 0.16100132 |
| CD3D | | | | | | | | | |
| HLA-C)- | −0.3615402 | −0.2500082 | 0.39992175 | 0.08735178 | 0.0223822 | 0.00474308 | −0.1158725 | 0.08221344 | 0.02911726 |
| CD3D | | | | | | | | | |
| HLA-B)- | −0.363846 | −0.2674001 | 0.37654841 | −0.1645586 | 0.31341687 | −0.2619236 | 0.17304943 | −0.1335968 | −0.1645586 |
| CD3D | | | | | | | | | |
| GAS6)- | −0.2807642 | 0.02305665 | 0.20521098 | 0.20092227 | 0.02384795 | 0.17891963 | 0.15922791 | −0.1043478 | 0.11462451 |
| AXL | | | | | | | | | |
| PROS1)- | −0.079718 | 0.28250486 | 0.19683094 | −0.1125165 | −0.0451267 | 0.1428195 | 0.04334794 | 0.04808959 | 0.10316206 |
| AXL | | | | | | | | | |
| SPP1)- | 0.21868617 | 0.23360453 | −0.1624864 | −0.026614 | 0.01799486 | −0.11662 | 0.10480524 | −0.1874485 | −0.085639 |
| PTGER4 | | | | | | | | | |
| LRPAP1)- | −0.0874164 | 0.08415157 | 0.1816564 | −0.0640337 | −0.216592 | 0.28394492 | −0.0725488 | 0.03432374 | −0.0471689 |
| LRP8 | | | | | | | | | |
| SORBS1)- | 0.07665689 | −0.0614746 | 0.11794551 | −0.1729534 | 0.09606642 | −0.0942526 | 0.14480532 | 0.13964677 | 0.03405909 |
| INSR | | | | | | | | | |
| INS)- INSR | 0.22039875 | −0.4116484 | 0.31013013 | 0.15395764 | −0.0080374 | −0.0079742 | 0.29427847 | 0.17160369 | −0.4181818 |
| AHSG)- | 0.24732921 | −0.2118066 | 0.33548692 | 0.22326196 | −0.0887014 | −0.1946999 | 0.18262073 | 0.22835114 | −0.1397279 |
| INSR | | | | | | | | | |
| ARF1)- | 0.01799071 | −0.3818182 | 0.2413123 | −0.343083 | 0.14097961 | 0.02207651 | 0.00118581 | 0.1875515 | −0.0833992 |
| INSR | | | | | | | | | |
| GIP)- INSR | 0.11049597 | −0.1384899 | 0.15898534 | 0.34799223 | −0.1113124 | −0.0212878 | −0.0863693 | 0.07575458 | 0.18024309 |
| IGF2)- | 0.01917817 | −0.1649648 | 0.14836776 | −0.0744401 | −0.0422939 | 0.07242414 | 0.00111993 | 0.13035027 | 0.09196311 |
| INSR | | | | | | | | | |
| HRAS)- | 0.12085558 | −0.0829572 | 0.08658408 | 0.07345917 | −0.0183819 | −0.0818559 | 0.14453704 | 0.10399025 | −0.2090909 |
| INSR | | | | | | | | | |
| CALM1)- | 0.15137736 | −0.0890016 | 0.12141775 | 0.01818182 | 0.04776178 | 0.30887346 | 0.00830067 | −0.3387921 | −0.028195 |
| INSR | | | | | | | | | |
| CD86)- | −0.2010279 | −0.1226117 | 0.17343879 | 0.00816972 | −0.0777723 | 0.00724781 | 0.11572046 | −0.1044206 | 0.0516469 |
| CTLA4 | | | | | | | | | |
| F2)- GP9 | 0.09489917 | −0.0137713 | −0.0141722 | −0.0223994 | −0.0041521 | 0.22067012 | −0.1434738 | −0.0498781 | −0.0318851 |
| DKK1)- | 0.15736927 | 0.07353474 | 0.02950388 | 0.03669543 | −0.0026038 | 0.01008104 | 0.10381993 | −0.0867732 | −0.2416417 |
| KREMEN2 | | | | | | | | | |
| ACE2)- | 0.02777961 | −0.1383242 | −0.4080644 | 0.24640116 | 0.02271005 | 0.18557686 | −0.1342694 | 0.09776987 | −0.0682477 |
| SLC6A19 | | | | | | | | | |
| GHRH)- | −0.1295118 | 0.02748938 | 0.2383093 | 0.0668665 | −0.2151159 | 0.07434859 | −0.218211 | 0.16061664 | 0.31001318 |
| ADRB2 | | | | | | | | | |
| PTHLH)- | 0.03217513 | 0.25560398 | 0.16510894 | −0.1156861 | 0.30397097 | 0.13885229 | 0.07715932 | 0.05645958 | 0.03952569 |
| ADRB2 | | | | | | | | | |
| POMC)- | −0.026726 | 0.11651894 | 0.18670782 | 0.01475918 | −0.1779504 | 0.03624143 | −0.0143895 | 0.09355338 | 0.12391713 |
| ADRB2 | | | | | | | | | |
| HSPA8)- | −0.1601608 | 0.15092724 | 0.284819 | 0.13873518 | 0.22104923 | −0.0975031 | −0.1498963 | −0.1915146 | −0.1459816 |
| ADRB2 | | | | | | | | | |
| ADCYAP1)- | 0.01581288 | 0.06199559 | 0.10947936 | −0.1309089 | −0.1236387 | 0.13783561 | 0.11847816 | 0.2641149 | 0.25744401 |
| ADRB2 | | | | | | | | | |
| CALCA)- | −0.086091 | −0.1995254 | −0.0182611 | 0.3880853 | 0.10135371 | 0.02043036 | 0.10039292 | 0.04097767 | 0.25546772 |
| ADRB2 | | | | | | | | | |
| ADM)- | −0.1376231 | −0.1699338 | 0.17845144 | −0.0656829 | 0.01320178 | 0.19333904 | 0.21366376 | 0.04394374 | 0.07694588 |
| ADRB2 | | | | | | | | | |
| GCG)- | −0.1452245 | −0.0754406 | 0.17391763 | 0.23270523 | −0.2535569 | 0.20102787 | 0.12534264 | −0.1001383 | 0.08998682 |
| ADRB2 | | | | | | | | | |
| ADM2)- | 0.02596119 | 0.10489211 | 0.07354118 | 0.08748353 | −0.0187453 | 0.29725278 | 0.23808624 | −0.2642224 | 0.20369577 |
| ADRB2 | | | | | | | | | |
| NPS)- | −0.2704082 | −0.3402324 | 0.15850916 | 0.31146245 | −0.0071945 | 0.21516569 | 0.06514649 | −0.0910468 | 0.06587615 |
| ADRB2 | | | | | | | | | |
| FSHB)- | −0.1417792 | −0.0583158 | 0.31334016 | −0.0875638 | −0.0797136 | −0.034593 | 0.07471715 | 0.05619606 | 0.52279315 |
| ADRB2 | | | | | | | | | |
| CALCB)- | −0.0980166 | 0.04348686 | 0.15075118 | 0.11030211 | −0.0045884 | 0.32522817 | −0.0543217 | 0.33414586 | 0.41844532 |
| ADRB2 | | | | | | | | | |
| ACTR2)- | −0.2402398 | 0.08835447 | 0.13991968 | −0.0607378 | 0.00706249 | −0.1222742 | −0.2791992 | −0.2002767 | −0.4043478 |
| ADRB2 | | | | | | | | | |
| GIP)- | −0.0634183 | 0.06364475 | 0.16457102 | −0.0267484 | −0.0452176 | 0.15915006 | −0.0398021 | 0.14635834 | 0.08491716 |
| ADRB2 | | | | | | | | | |
| INSL3)- | −0.0140061 | −0.1171159 | 0.18948745 | −0.0279315 | −0.1349793 | 0.16094604 | 0.09742271 | 0.07319323 | 0.24782609 |
| ADRB2 | | | | | | | | | |
| PENK)- | −0.056365 | 0.33569577 | 0.00016487 | 0.24330467 | −0.2071704 | 0.15817906 | 0.13496327 | −0.0167325 | 0.18940018 |
| MRGPRX1 | | | | | | | | | |
| NMS)- | −0.0946602 | −0.039574 | −0.148279 | 0.15046113 | 0.11075222 | 0.14310186 | −0.103171 | 0.0586375 | 0.21541502 |
| NMUR1 | | | | | | | | | |
| CD6)- | 0.04289019 | 0.03929531 | −0.032801 | −0.0239805 | 0.0815076 | −0.0682499 | 0.00882915 | −0.0629776 | 0.00764189 |
| ALCAM | | | | | | | | | |
| S100A9)- | −0.3778744 | −0.3937008 | 0.3531699 | 0.31476663 | −0.2198722 | 0.13675889 | −0.1808658 | 0.21633729 | 0.32728351 |
| ALCAM | | | | | | | | | |
| TSLP)- | −0.189679 | −0.0430356 | −0.0231408 | −0.0192365 | −0.0676816 | 0.06410172 | 0.02359299 | 0.11054747 | 0.05507428 |
| IL7R | | | | | | | | | |
| LAMA3)- | −0.4274045 | 0.31330698 | 0.34624506 | 0.27588933 | 0.29894598 | −0.4457181 | −0.1660079 | 0.5372859 | −0.2256917 |
| SDC2 | | | | | | | | | |
| LAMA1)- | −0.0508039 | −0.099038 | 0.02490775 | −0.1212361 | −0.2203993 | 0.02187521 | −0.0396574 | −0.1035573 | 0.04637681 |
| SDC2 | | | | | | | | | |
| HRAS)- | −0.1543175 | 0.18317794 | 0.17217409 | −0.0839345 | −0.1011958 | 0.35234048 | 0.04361001 | 0.1629776 | −0.0725955 |
| SDC2 | | | | | | | | | |
| SERPINC1)- | 0.00659326 | −0.1680623 | −0.1645679 | 0.00329446 | 0.00540291 | 0.25953746 | −0.1157482 | −0.0621891 | −0.1500049 |
| SDC2 | | | | | | | | | |
| FN1)- SDC2 | −0.2988142 | 0.50434783 | 0.55256917 | −0.1930171 | −0.1801054 | 0.22648221 | 0.57509881 | −0.2905138 | 0.5256917 |
| GNAI2)- | −0.2709809 | −0.0414443 | −0.0134458 | 0.12951252 | −0.1909061 | 0.26697414 | −0.0518616 | 0.00981716 | −0.0549407 |
| P2RY12 | | | | | | | | | |
| PLAU)- | −0.2902801 | 0.19207328 | 0.14930979 | −0.1687144 | −0.1104779 | 0.40420345 | 0.35098814 | 0.12814178 | 0.46666667 |
| MRC2 | | | | | | | | | |
| CALM1)- | 0.1946961 | 0.10066539 | −0.0468457 | −0.245792 | −0.0797206 | 0.00224008 | 0.15331401 | −0.0193701 | 0.14216542 |
| GP6 | | | | | | | | | |
| XCL1)- | 0.11441755 | −0.1990443 | 0.06479041 | 0.01080369 | 0.10270431 | 0.12490119 | 0.17266708 | 0.11976285 | −0.1434783 |
| XCR1 | | | | | | | | | |
| CXCL2)- | 0.31499103 | −0.3592536 | 0.38070378 | −0.1350461 | 0.21706907 | 0.34677207 | −0.5060939 | −0.4056787 | 0.43170065 |
| XCR1 | | | | | | | | | |
| CD200)- | −0.1823334 | 0.10641453 | −0.2040817 | 0.04150334 | 0.07383725 | 0.087031 | −0.0672117 | −0.040979 | 0.13162489 |
| CD200R1 | | | | | | | | | |
| CALM1)- | −0.0709533 | 0.00164696 | 0.1108402 | −0.1833992 | −0.0591084 | −0.0530979 | 0.19248131 | −0.0196317 | 0.1571805 |
| AQP1 | | | | | | | | | |
| RARRES2)- | −0.4636364 | −0.2841897 | 0.21057496 | 0.28651426 | −0.3386959 | 0.4842875 | 0.14636081 | −0.3932806 | −0.0753623 |
| CMKLR1 | | | | | | | | | |
| C4B)- CD46 | 0.16098848 | −0.1907147 | 0.06530478 | 0.26839713 | 0.0966435 | −0.031951 | 0.01225296 | 0.04743083 | −0.1809678 |
| IL24)- | −0.0644396 | 0.01937078 | 0.09661263 | 0.16997727 | 0.13136738 | −0.1755172 | 0.0670796 | 0.04361289 | 0.15935966 |
| IL20RA | | | | | | | | | |
| EDN3)- | −0.1455854 | −0.0680057 | 0.06688439 | −0.0550725 | −0.1072464 | −0.1492193 | 0.26785244 | −0.326899 | 0.13096179 |
| EDNRA | | | | | | | | | |
| EDN1)- | −0.0731748 | 0.08631482 | 0.11458128 | −0.1174068 | 0.01159573 | 0.11425183 | −0.0251647 | 0.24830358 | 0.02582345 |
| EDNRA | | | | | | | | | |
| SEMA4A)- | −0.0415774 | 0.45590223 | 0.13014398 | −0.0172596 | 0.05949009 | −0.1247694 | −0.1178602 | 0.21251647 | −0.271805 |
| PLXND1 | | | | | | | | | |
| SEMA4D)- | −0.2619267 | 0.24261993 | −0.1389694 | −0.3296443 | 0.26034256 | 0.38616601 | 0.16231884 | 0.23662714 | −0.314888 |
| MET | | | | | | | | | |
| SEMA5A)- | 0.22313723 | −0.2931682 | 0.21523157 | 0.3687542 | −0.284509 | −0.2471985 | 0.28418972 | 0.23465086 | −0.3320158 |
| MET | | | | | | | | | |
| TNF)- ICOS | −0.1049578 | −0.0709388 | 0.1880297 | 0.31816536 | −0.3274771 | 0.53306537 | 0.17516229 | −0.0831357 | 0.04374177 |
| ICOSLG)- | −0.2143751 | −0.1295869 | 0.19672456 | 0.14718672 | −0.2263305 | 0.21531164 | −0.245873 | −0.1155468 | −0.0820817 |
| ICOS | | | | | | | | | |
| CXCL2)- | −0.0306225 | 0.1126532 | −0.1628086 | 0.03939913 | −0.2513522 | 0.18894525 | 0.27926015 | −0.1113417 | −0.1129303 |
| CXCR2 | | | | | | | | | |
| CXCL1)- | 0.10225663 | 0.1377788 | −0.0544507 | 0.12715773 | 0.01444401 | 0.06080769 | −0.1106861 | 0.18007511 | 0.1270427 |
| CXCR2 | | | | | | | | | |
| CD55)- | −0.1228631 | −0.187747 | 0.1237976 | −0.2599473 | 0.0308341 | −0.1027702 | −0.1849387 | −0.0083665 | 0.01976285 |
| ADGRE2 | | | | | | | | | |
| IL17F)- | −0.184735 | −0.1755114 | 0.10142063 | 0.03202108 | −0.0701454 | 0.22355461 | −0.075407 | 0.05480896 | 0.25942029 |
| IL17RA | | | | | | | | | |
| SEMA4B)- | −0.0576435 | −0.3318841 | 0.17325428 | −0.3573123 | 0.16587615 | −0.0146909 | 0.05626009 | −0.0249028 | 0.3534372 |
| DCBLD2 | | | | | | | | | |
| GHRH)- | −0.1610522 | 0.21549826 | 0.04816415 | −0.1195033 | 0.06741835 | 0.1340668 | −0.1283709 | 0.20099476 | 0.16640316 |
| PTH1R | | | | | | | | | |
| PTHLH)- | −0.2211308 | 0.2174315 | 0.38756186 | 0.10218064 | 0.19451411 | 0.01413183 | 0.07035013 | 0.14736981 | −0.1177866 |
| PTH1R | | | | | | | | | |
| POMC)- | −0.2239505 | 0.19435201 | −0.0271429 | −0.134941 | 0.09222503 | 0.06984483 | −0.2891242 | 0.08913631 | 0.04058105 |
| PTH1R | | | | | | | | | |
| ADCYAP1)- | −0.1255765 | −0.0212801 | −0.0549603 | −0.0091577 | 0.08321522 | 0.12633417 | −0.1612053 | 0.11844922 | 0.44137022 |
| PTH1R | | | | | | | | | |
| CALCA)- | 0.01924789 | −0.094852 | −0.095824 | 0.27038784 | 0.22738518 | −0.1196455 | 0.17676541 | −0.0605422 | 0.09802372 |
| PTH1R | | | | | | | | | |
| ADM)- | −0.113864 | −0.0056008 | 0.18313717 | 0.21997497 | −0.034551 | 0.13189709 | 0.10882541 | −0.0020423 | 0.30817879 |
| PTH1R | | | | | | | | | |
| GCG)- | −0.0409831 | 0.26012189 | 0.33044289 | 0.10172618 | −0.0390373 | 0.1074617 | 0.17696321 | −0.1345894 | 0.04532279 |
| PTH1R | | | | | | | | | |
| ADM2)- | −0.2292943 | −0.0013836 | 0.11589571 | 0.11185771 | 0.18586412 | −0.0006588 | 0.14459504 | −0.0272745 | 0.09591884 |
| PTH1R | | | | | | | | | |
| NPS)- | 0.22432051 | −0.0063373 | 0.019854 | 0.31185771 | 0.03665855 | −0.1036266 | −0.1946991 | 0.19249646 | 0.42990777 |
| PTH1R | | | | | | | | | |
| FSHB)- | −0.1382492 | 0.011136 | 0.30986618 | 0.07557239 | −0.4766396 | 0.16959874 | −0.0716028 | 0.11317896 | 0.26890646 |
| PTH1R | | | | | | | | | |
| CALCB)- | 0.0678022 | −0.0125189 | 0.07240835 | 0.1622245 | 0.22013392 | −0.0464549 | 0.10667901 | −0.2728021 | 0.02121212 |
| PTH1R | | | | | | | | | |
| GIP)- | −0.247801 | 0.42344183 | 0.25891863 | −0.1059393 | −0.1761235 | 0.12603769 | 0.08986915 | 0.10152184 | 0.09684113 |
| PTH1R | | | | | | | | | |
| INSL3)- | −0.1565332 | −0.0036249 | 0.02114116 | 0.01831357 | 0.01562604 | 0.31167035 | 0.10634935 | −0.0305675 | 0.2400527 |
| PTH1R | | | | | | | | | |
| PDX1)- | −0.1712638 | 0.40833007 | 0.15580092 | −0.1236285 | −0.1085989 | 0.03097608 | 0.19429344 | −0.0173277 | −0.0986857 |
| SLC2A2 | | | | | | | | | |
| CD86)- | 0.02273102 | −0.0103772 | 0.10615445 | 0.21498929 | −0.070572 | 0.02187449 | 0.06720253 | 0.08682763 | 0.18492045 |
| CD28 | | | | | | | | | |
| GHRH)- | −0.1035995 | −0.241792 | −0.125676 | −0.0044143 | 0.08397048 | −0.0123197 | 0.03717252 | 0.07305906 | 0.00013177 |
| VIPR1 | | | | | | | | | |
| PTHLH)- | −0.1370035 | −0.256882 | −0.1758847 | −0.0106078 | −0.1837656 | 0.15745956 | −0.2522327 | 0.3306433 | 0.12728531 |
| VIPR1 | | | | | | | | | |
| POMC)- | 0.03598498 | −0.2482863 | −0.1052354 | −0.0302132 | 0.22818815 | 0.10858894 | 0.00626154 | 0.09065156 | 0.14751614 |
| VIPR1 | | | | | | | | | |
| ADCYAP1)- | 0.01653709 | −0.1787244 | 0.16192078 | 0.05673058 | 0.06927692 | 0.15094215 | −0.1391992 | 0.05797292 | −0.0517179 |
| VIPR1 | | | | | | | | | |
| CALCA)- | 0.04587852 | 0.00876759 | 0.28430112 | 0.11322745 | −0.1993143 | 0.01103862 | 0.01621355 | 0.32543891 | −0.1950786 |
| VIPR1 | | | | | | | | | |
| ADM)- | 0.09666579 | −0.349687 | 0.41779288 | −0.0537309 | −0.0694724 | −0.0536944 | −0.0726668 | 0.1475723 | −0.0033601 |
| VIPR1 | | | | | | | | | |
| GCG)- | 0.21232787 | −0.0252372 | 0.32920239 | 0.3745923 | −0.1270888 | −0.2108384 | −0.1637832 | 0.275042 | 0.01495536 |
| VIPR1 | | | | | | | | | |
| ADM2)- | −0.0283982 | 0.04546653 | 0.2496045 | 0.10607109 | −0.1147471 | 0.07892223 | −0.0608358 | 0.20034258 | −0.2819871 |
| VIPR1 | | | | | | | | | |
| GNAS)- | 0.17694917 | 0.13552064 | −0.0105454 | 0.06611761 | 0.15565371 | −0.0670027 | −0.0602406 | −0.0052703 | −0.0017788 |
| VIPR1 | | | | | | | | | |
| NPS)- | −0.1734556 | −0.1842943 | 0.19909542 | 0.00224001 | 6.59E−05 | 0.23367041 | 0.03638162 | 0.04907935 | −0.0806404 |
| VIPR1 | | | | | | | | | |
| FSHB)- | −0.0266878 | −0.1053741 | 0.01018558 | −0.126219 | −0.0295979 | −0.0751795 | −0.1327402 | 0.17589512 | 0.0019106 |
| VIPR1 | | | | | | | | | |
| PTMA)- | 0.12055733 | 0.01627302 | 0.10828803 | 0.13776065 | 0.0740155 | 0.15000494 | 0.25664857 | −0.1330083 | −0.3173568 |
| VIPR1 | | | | | | | | | |
| CALM1)- | 0.15478622 | −0.2130716 | 0.06713025 | 0.32420859 | 0.07118142 | −0.0133733 | 0.05404515 | 0.23228697 | −0.4108443 |
| VIPR1 | | | | | | | | | |
| CALCB)- | −0.2166507 | 0.01993345 | −0.1245262 | −0.0879736 | −0.0848771 | 0.21843701 | 0.10453124 | 0.22490859 | −0.1052805 |
| VIPR1 | | | | | | | | | |
| GIP)- | −0.1537144 | 0.05805028 | −0.0746515 | 0.06285827 | 0.16811681 | 0.30415733 | −0.0743475 | 0.16568944 | −0.0424957 |
| VIPR1 | | | | | | | | | |
| INSL3)- | 0.2486077 | 0.00204331 | 0.093172 | −0.1573937 | −0.1051903 | −0.2017194 | −0.0370407 | 0.05474489 | 0.0345884 |
| VIPR1 | | | | | | | | | |
| IL24)- | 0.10594281 | −0.3339043 | 0.33788059 | −0.1564003 | 0.27370676 | −0.0144937 | 0.10075119 | −0.3445323 | 0.57496706 |
| IL20RB | | | | | | | | | |
| TNFSF18)- | −0.0448794 | −0.1198063 | −0.008908 | 0.36066546 | 0.04980872 | 0.08262502 | −0.1706466 | 0.32419791 | 0.19308936 |
| TNFRSF18 | | | | | | | | | |
| F11)- | 0.20823865 | −0.3162618 | −0.0097087 | 0.17891963 | 0.0081797 | 0.27366757 | 0.16748589 | 0.00553396 | 0.26152833 |
| GP1BA | | | | | | | | | |
| APOD)- | −0.0071156 | −0.074179 | 0.07336276 | −0.2109354 | −0.3227592 | 0.31267912 | −0.0330731 | −0.0780051 | 0.08432148 |
| LEPR | | | | | | | | | |
| DKK1)- | 0.06820204 | 0.12927882 | −0.0568811 | 0.29461756 | 0.18366339 | −0.0058638 | 0.3143328 | 0.0266166 | 0.02134458 |
| KREMEN1 | | | | | | | | | |
| DKK3)- | −0.1393418 | 0.06607596 | 0.41489294 | −0.1927334 | 0.08744934 | −0.0885492 | 0.31472819 | −0.1218829 | 0.25310452 |
| KREMEN1 | | | | | | | | | |
| BSG)- | −0.0607498 | −0.1185888 | 0.02265833 | −0.2006654 | 0.06055014 | −0.1073923 | −0.012796 | −0.1235999 | −0.0306334 |
| SELE | | | | | | | | | |
| AFDN)- | 0.33006127 | −0.2421687 | 0.09381286 | −0.1172596 | 0.08273455 | 0.20745084 | −0.2135347 | −0.1603531 | 0.31015514 |
| EPHB3 | | | | | | | | | |
| CXCL12)- | −0.0299964 | −0.1526849 | 0.28199803 | 0.10349825 | 0.1329178 | 0.06107122 | 0.24325422 | −0.2137022 | 0.0027668 |
| CCR4 | | | | | | | | | |
| CCL5)- | −0.3392634 | −0.1160967 | 0.45196027 | −0.0212121 | −0.3379301 | 0.30263505 | −0.3980344 | 0.24110672 | −0.0339921 |
| CCR4 | | | | | | | | | |
| NTN4)- | −0.0097516 | 0.01199104 | 0.20941649 | −0.0671937 | 0.1977269 | −0.0019764 | −0.1249876 | 0.00177877 | −0.1408432 |
| UNC5A | | | | | | | | | |
| FAM3C)- | 0.21317523 | −0.4471673 | 0.15540696 | −0.4324111 | 0.19987483 | 0.30803689 | 0.25567377 | 0.20316206 | 0.03478261 |
| LAMP1 | | | | | | | | | |
| POMC)- | −0.0462664 | 0.13714701 | 0.28488971 | −0.0369638 | −0.0427384 | −0.1438408 | 0.06330578 | −0.064497 | −0.0544155 |
| MC2R | | | | | | | | | |
| F10)- F3 | −0.0906362 | 0.08485581 | −0.026181 | −0.3148993 | 0.06079208 | 0.18242987 | 0.10569002 | 0.01324198 | −0.0270769 |
| IL6)- F3 | 0.11762383 | 0.23752233 | 0.03972286 | −0.1194531 | −0.1233722 | −0.3024872 | −0.0395925 | 0.09341854 | −0.0799789 |
| PTN)- | 0.09146925 | −0.3053808 | 0.16976654 | 0.27869285 | −0.0799183 | −0.0388721 | −0.0700841 | 0.45665349 | 0.05783926 |
| PTPRB | | | | | | | | | |
| EFNA3)- | 0.19060173 | −0.0983855 | −0.0993963 | 0.01930235 | 0.00267133 | 0.06951079 | 0.13646417 | 0.12887074 | −0.0673254 |
| EPHA3 | | | | | | | | | |
| EFNA1)- | 0.04822929 | 0.12273132 | −0.1349186 | 0.23280632 | 0.03212086 | −0.0521808 | 0.14431301 | 0.19804981 | −0.1334651 |
| EPHA3 | | | | | | | | | |
| EFNA4)- | −0.0430986 | −0.1581392 | −0.0440362 | −0.0478324 | 0.0488143 | −0.0065894 | −0.0081127 | −0.1900119 | −0.0552042 |
| EPHA3 | | | | | | | | | |
| NRG1)- | 0.1008836 | 0.08215749 | 0.22077839 | 0.1421701 | −0.2435576 | 0.16832466 | −0.181297 | 0.15230567 | −0.0115942 |
| MS4A4A | | | | | | | | | |
| CCN1)- | −0.0216074 | 0.00658762 | 0.21067888 | 0.15783926 | −0.2835403 | −0.0779974 | 0.29368556 | −0.0220026 | −0.0084321 |
| CAV1 | | | | | | | | | |
| PTGS2)- | −0.1490049 | −0.1088045 | 0.00042838 | 0.16086957 | 0.11317896 | −0.0757576 | −0.11588 | −0.1205534 | 0.26047431 |
| CAV1 | | | | | | | | | |
| HRAS)- | −0.1189339 | 0.01324416 | 0.00230627 | −0.0644991 | 0.02879167 | 0.20403861 | 0.07655061 | 0.24137022 | −0.1252964 |
| CAV1 | | | | | | | | | |
| PLAU)- | −0.3376932 | −0.2976971 | 0.00207606 | 0.07661649 | −0.0500461 | 0.5224151 | 0.13290285 | 0.01791831 | 0.07483531 |
| PLAUR | | | | | | | | | |
| MMP12)- | −0.0982364 | −0.0320422 | 0.05569112 | 0.15606575 | 0.04451107 | 0.11956916 | −0.0479689 | 0.22411067 | 0.24216074 |
| PLAUR | | | | | | | | | |
| FN1)- | −0.3131752 | −0.1631094 | 0.13151913 | 0.32108037 | 0.0792014 | 0.49907773 | 0.18541825 | −0.13083 | −0.0155468 |
| PLAUR | | | | | | | | | |
| WNT5B)- | −0.098929 | −0.0968603 | 0.03166601 | 0.17623017 | −0.1382632 | 0.03496505 | 0.13019701 | 0.13324108 | 0.15191541 |
| KLRG2 | | | | | | | | | |
| FGF21)- | −0.1651068 | 0.13222162 | 0.05972905 | 0.31568892 | −0.2142128 | −0.001581 | −0.0150567 | −0.2832768 | 0.41722116 |
| FGFR3 | | | | | | | | | |
| FGF14)- | 0.16434127 | 0.09662212 | −0.1093112 | −0.2619613 | 0.14857294 | 0.23691062 | −0.1561625 | −0.1389987 | 0.20606739 |
| FGFR3 | | | | | | | | | |
| FGF17)- | −0.1562541 | 0.2107622 | 0.06726871 | −0.013242 | −0.3192541 | 0.18189005 | 0.0235891 | 0.03675889 | 0.25929708 |
| FGFR3 | | | | | | | | | |
| FGF12)- | −0.1695061 | 0.13708562 | −0.2346078 | −0.068518 | 0.05143158 | 0.12543646 | −0.0975851 | 0.23952569 | 0.03577193 |
| FGFR3 | | | | | | | | | |
| NRG1)- | −0.2327652 | 0.43050262 | 0.2974774 | 0.17715265 | 0.22536325 | −0.0242448 | −0.0381511 | 0.13946441 | −0.0573123 |
| ADGRL1 | | | | | | | | | |
| LAMB3)- | 0.41093544 | −0.2541502 | 0.41146245 | −0.2646904 | 0.41554677 | 0.45783926 | −0.1471673 | −0.1957839 | 0.12990777 |
| COL17A1 | | | | | | | | | |
| UCN3)- | −0.3399783 | 0.18148869 | 0.32604898 | −0.2027935 | −0.2493245 | 0.2884438 | −0.0262282 | 0.08656412 | 0.41200303 |
| CRHR2 | | | | | | | | | |
| NPPC)- | −0.0749267 | 0.18174634 | −0.0628238 | −0.0625288 | −0.1645733 | −0.1709117 | 0.0305877 | 0.06140669 | −0.0337286 |
| NPR2 | | | | | | | | | |
| RGMA)- | −0.0487017 | 0.13067546 | 0.01861983 | −0.3978916 | −0.0575637 | 0.12413929 | −0.0210845 | −0.1385467 | 0.214361 |
| NEO1 | | | | | | | | | |
| TNF)- | −0.2177311 | −0.0851794 | −0.0645715 | −0.1942733 | 0.22336498 | 0.03308727 | −0.2519524 | −0.030308 | −0.2704875 |
| TRADD | | | | | | | | | |
| ADIPOQ)- | −0.0193701 | −0.0510278 | −0.1549895 | 0.18433969 | 0.03381117 | 0.04041901 | 0.22068808 | −0.1154885 | −0.0505303 |
| ADIPOR1 | | | | | | | | | |
| ANXA1)- | 0.21238472 | 0.06534914 | −0.0785154 | 0.07891963 | −0.0566287 | 0.17971014 | 0.05062959 | −0.21278 | −0.0847167 |
| FPR3 | | | | | | | | | |
| GHRH)- | 0.0598688 | −0.1347156 | 0.49953833 | −0.0805718 | −0.3134393 | 0.02049221 | −0.1591696 | 0.11128682 | 0.10382424 |
| ADRB3 | | | | | | | | | |
| PTHLH)- | 0.32159584 | 0.08551181 | 0.35843147 | 0.1797279 | −0.3355964 | 0.26588034 | −0.1714945 | 0.42281084 | 0.23564676 |
| ADRB3 | | | | | | | | | |
| POMC)- | −0.0022412 | 0.10238582 | 0.04338652 | −0.0204922 | −0.1073206 | 0.01439963 | −0.1825732 | 0.11326722 | 0.08188945 |
| ADRB3 | | | | | | | | | |
| ADCYAP1)- | 0.0734078 | 0.30893398 | 0.18172831 | 0.17294769 | −0.1171314 | 0.04072353 | 0.04712474 | −0.0833498 | 0.14763332 |
| ADRB3 | | | | | | | | | |
| ADM)- | −0.0251755 | 0.03736162 | 0.05679346 | 0.00945416 | −0.1768448 | 0.27892726 | 0.034768 | 0.01851547 | −0.0822847 |
| ADRB3 | | | | | | | | | |
| GCG)- | −0.1000362 | 0.20333399 | −0.0221819 | −0.046055 | −0.0528658 | 0.22359143 | 0.04165431 | −0.0253673 | 0.10942389 |
| ADRB3 | | | | | | | | | |
| ADM2)- | 0.14458467 | −0.1085524 | 0.05553725 | −0.0190388 | −0.0092272 | 0.12927456 | 0.05875956 | −0.0875037 | 0.23041702 |
| ADRB3 | | | | | | | | | |
| GNAS)- | −0.4852738 | −0.0304358 | −0.0572088 | −0.037619 | −0.1261576 | −0.3193859 | −0.0454111 | 0.11326349 | 0.06792055 |
| ADRB3 | | | | | | | | | |
| NPS)- | −0.0054142 | −0.0362425 | 0.27415661 | 0.1428242 | −0.1932444 | −0.1582658 | −0.1763058 | 0.03841339 | 0.11126849 |
| ADRB3 | | | | | | | | | |
| FSHB)- | 0.27057932 | 0.07479161 | −0.137357 | 0.077288 | −0.0341463 | 0.06642723 | 0.12502885 | 0.07320287 | 0.12628874 |
| ADRB3 | | | | | | | | | |
| CALCB)- | 0.10999077 | −0.0371298 | 0.23791161 | 0.20878361 | 0.01704133 | 0.23007217 | 0.19798981 | 0.28266456 | 0.16173128 |
| ADRB3 | | | | | | | | | |
| GIP)- | 0.0214827 | −0.1028826 | 0.30493394 | 0.30274081 | −0.3058797 | 0.25653191 | −0.0503559 | 0.07195335 | 0.17010343 |
| ADRB3 | | | | | | | | | |
| INSL3)- | 0.0276203 | 0.04396112 | 0.05538888 | 0.11258605 | −0.0759928 | 0.20623312 | 0.26370081 | −0.0208869 | 0.01633782 |
| ADRB3 | | | | | | | | | |
| IFNA16)- | 0.07146862 | 0.0060656 | 0.05137168 | −0.0752306 | 0.05139525 | 0.23346509 | −0.1225619 | −0.1589644 | 0.00823479 |
| IFNAR2 | | | | | | | | | |
| IFNA14)- | −0.1684349 | −0.0463262 | −0.1162377 | −0.0280743 | −0.1490722 | −0.0430341 | −0.0975192 | 0.06258235 | −0.0019763 |
| IFNAR2 | | | | | | | | | |
| IFNA4)- | −0.2422406 | 0.0904444 | −0.1174861 | −0.3301483 | 0.31196652 | 0.34688635 | 0.32075908 | 0.07918314 | −0.3889328 |
| IFNAR2 | | | | | | | | | |
| IFNA13)- | −0.0050768 | 0.06263601 | −0.3456656 | −0.0555995 | 0.31746451 | 0.01501976 | 0.24933286 | −0.0841897 | −0.0949934 |
| IFNAR2 | | | | | | | | | |
| IFNB1)- | −0.2767599 | 0.15291328 | −0.2222369 | 0.27813828 | 0.05363732 | 0.21403867 | −0.095213 | −0.1650856 | −0.1075099 |
| IFNAR2 | | | | | | | | | |
| IFNA8)- | 0.17973169 | 0.15855963 | −0.203391 | 0.02220099 | 0.04797048 | −0.2781383 | 0.28814286 | 0.18366271 | −0.214361 |
| IFNAR2 | | | | | | | | | |
| IFNE)- | −0.1436668 | −0.0819164 | −0.2419169 | −0.2249012 | 0.10450368 | 0.15849802 | 0.3089747 | 0.12371949 | 0.18571099 |
| IFNAR2 | | | | | | | | | |
| IFNA2)- | −0.3226977 | −0.2240096 | −0.0517293 | 0.17076786 | −0.1007578 | 0.02688013 | 0.21105694 | 0.16212655 | −0.1907177 |
| IFNAR2 | | | | | | | | | |
| MDK)- | −0.2305665 | 0.06403162 | 0.20172726 | 0.08155468 | 0.05610127 | −0.0155468 | −0.0216238 | 0.29790178 | −0.2596923 |
| SDC3 | | | | | | | | | |
| COL5A1)- | 0.20764163 | 0.11607378 | −0.0581449 | 0.25633255 | 0.11497514 | −0.1974373 | 0.02280968 | 0.18550725 | 0.20790514 |
| SDC3 | | | | | | | | | |
| SHBG)- | −0.0126553 | −0.0271562 | 0.0573576 | 0.3057413 | 0.01093832 | −0.1326789 | −0.0098178 | 0.2 | −0.0604743 |
| SLC37A1 | | | | | | | | | |
| CD24)- | 0.15322793 | 0.16758893 | −0.1892801 | 0.22121212 | −0.1955433 | 0.23372859 | 0.08827798 | −0.01278 | 0.0715415 |
| SIGLEC10 | | | | | | | | | |
| LAMA2)- | 0.24844352 | −0.0983596 | 0.35042988 | 0.21594203 | −0.3972463 | −0.2976383 | 0.4430976 | 0.41457229 | −0.2681159 |
| RPSA | | | | | | | | | |
| LAMA1)- | 0.02734671 | 0.113864 | 0.0977892 | −0.1694011 | 0.03848055 | −0.1652555 | 0.04018578 | −0.0789881 | 0.056917 |
| RPSA | | | | | | | | | |
| LAMB2)- | 0.13142725 | 0.01963109 | 0.06607596 | −0.084198 | −0.0874292 | −0.2250626 | 0.12773807 | 0.09861985 | 0.16192358 |
| RPSA | | | | | | | | | |
| EDN1)- | 0.2931506 | −0.122488 | −0.1996705 | 0.19686388 | −0.1341592 | 0.09079229 | 0.08828568 | −0.266939 | −0.0573123 |
| KEL | | | | | | | | | |
| NCAM1)- | −0.1203097 | −0.1095629 | 0.14568642 | −0.1228671 | 0.01041289 | 0.23375939 | 0.34683188 | 0.02615456 | 0.08880105 |
| GFRA1 | | | | | | | | | |
| GDNF)- | −0.2525452 | 0.10081376 | 0.2276053 | 0.18972957 | 0.1196784 | 0.1242547 | −0.0061289 | 0.42428435 | −0.2684542 |
| GFRA1 | | | | | | | | | |
| ARTN)- | −0.0569433 | −0.0514696 | 0.12525958 | 0.22168056 | −0.1565836 | −0.2172151 | −0.071172 | −0.0670663 | 0.04229249 |
| GFRA1 | | | | | | | | | |
| PTPN6)- | −0.0538315 | 0.00013178 | −0.0534208 | 0.0047434 | 0.13364527 | 0.11989855 | −0.0748954 | 0.34803518 | 0.08353646 |
| CLEC12A | | | | | | | | | |
| GDNF)- | −0.0713627 | 0.10410833 | 0.15289775 | 0.02865707 | −0.0245221 | 0.02187232 | −0.1601187 | −0.1815666 | 0.28637307 |
| RET | | | | | | | | | |
| ARTN)- | −0.0903549 | −0.2369843 | 0.18559795 | −0.0068513 | −0.0927489 | −0.2780816 | 0.14732543 | −0.151059 | 0.12753623 |
| RET | | | | | | | | | |
| VEGFA)- | −0.0527685 | 0.07747036 | 0.17942721 | 0.19512516 | −0.0865496 | 0.15672453 | 0.0457482 | 0.01179261 | 0.04809118 |
| RET | | | | | | | | | |
| TNFSF14)- | −0.1162622 | −0.303462 | 0.35748936 | 0.11568615 | −0.2417989 | −0.0588681 | −0.0928094 | 0.3310054 | −0.1113051 |
| TNFRSF14 | | | | | | | | | |
| LTA)- | −0.078571 | −0.0694042 | −0.0177788 | 0.21167595 | −0.2105455 | 0.24200191 | 0.12739027 | 0.1208288 | 0.05619421 |
| TNFRSF14 | | | | | | | | | |
| FASLG)- | 0.21122602 | −0.0842282 | −0.0176732 | −0.0935441 | −0.0007247 | −0.0150217 | 0.00329413 | −0.0876268 | 0.03280632 |
| FAS | | | | | | | | | |
| TNF)- FAS | −0.0563102 | −0.2219146 | 0.13114433 | −0.298659 | −0.078989 | −0.0199044 | 0.31346971 | −0.0581104 | −0.1459816 |
| CALM1)- | −0.0969198 | −0.2523799 | 0.09586243 | 0.39328063 | 0.08044273 | −0.1470549 | −0.0635109 | 0.23863487 | −0.4511199 |
| FAS | | | | | | | | | |
| PIGA)- | 0.14047109 | −0.0453961 | 0.24094878 | −0.1150198 | 0.00131752 | 0.03096179 | −0.084585 | −0.0491436 | 0.12108037 |
| PIGR | | | | | | | | | |
| ICAM2)- | −0.159483 | −0.0679353 | 0.17917839 | −0.1977863 | 0.07330741 | −0.2034524 | −0.1363127 | 0.1256917 | 0.19367589 |
| ITGAL | | | | | | | | | |
| PLAU)- | −0.0886956 | −0.024841 | 0.02870041 | −0.2691129 | −0.190222 | 0.40992193 | −0.0096854 | 0.04170235 | −0.0903821 |
| IGF2R | | | | | | | | | |
| IGF2)- | 0.0799183 | −0.0820212 | 0.02375383 | −0.3089592 | 0.35322023 | 0.41603531 | 0.00434854 | 0.12978457 | 0.02529644 |
| IGF2R | | | | | | | | | |
| ADIPOQ)- | 0.35477814 | 0.25757676 | −0.1358993 | −0.229074 | −0.0885096 | 0.00434811 | −0.2624885 | 0.03399321 | 0.08333882 |
| ADIPOR2 | | | | | | | | | |
| TNF)- | −0.1359486 | 0.09704644 | 0.00897009 | 0.10570365 | 0.04852321 | −0.0073151 | −0.008304 | −0.0224645 | 0.16534914 |
| PTPRS | | | | | | | | | |
| HSPG2)- | −0.1385421 | −0.0475626 | 0.18710253 | 0.16389987 | −0.2236795 | 0.24631905 | 0.14802123 | −0.1365658 | 0.03280632 |
| PTPRS | | | | | | | | | |
| RARRES2)- | −0.0065876 | −0.1147563 | −0.0207626 | −0.2156269 | 0.0912297 | 0.12253772 | −0.0458096 | 0.03162055 | −0.0196311 |
| CCRL2 | | | | | | | | | |
| CCL5)- | −0.1830401 | −0.1469988 | 0.37914101 | 0.27602108 | −0.4462974 | 0.14466403 | −0.4122203 | 0.09130435 | 0.24176548 |
| CCRL2 | | | | | | | | | |
| EFNA3)- | −0.3044582 | 0.32500825 | −0.0669522 | −0.0193682 | −0.1751046 | −0.0604783 | −0.2274045 | 0.05481076 | −0.2488801 |
| EPHA2 | | | | | | | | | |
| EFNA1)- | −0.2695171 | 0.45054185 | −0.2513917 | 0.46179183 | −0.2993412 | −0.271814 | 0.35915679 | 0.44843374 | −0.6359684 |
| EPHA2 | | | | | | | | | |
| EFNA4)- | −0.4137454 | 0.36708069 | −0.3218133 | 0.27908816 | −0.0880221 | −0.0125844 | 0.29538867 | 0.29559603 | −0.3928854 |
| EPHA2 | | | | | | | | | |
| SEMA4D)- | −0.2681866 | 0.34587508 | −0.3269408 | −0.3524374 | 0.28462248 | 0.22371542 | 0.22519436 | 0.12621871 | −0.227668 |
| PLXNB1 | | | | | | | | | |
| SEMA4A)- | 0.20742596 | −0.2008368 | 0.33174075 | −0.3283267 | 0.05455264 | 0.14044796 | −0.2942417 | −0.1121212 | 0.30395257 |
| PLXNB1 | | | | | | | | | |
| TGFB2)- | 0.15483956 | 0.06898369 | 0.02774208 | 0.39346423 | −0.4760148 | −0.1082523 | 0.12373579 | −0.0237829 | −0.2970454 |
| TGFBR2 | | | | | | | | | |
| S100A10)- | 0.07958101 | 0.35520422 | 0.07159104 | 0.34650856 | 0.00678994 | 0.01930235 | 0.05049607 | −0.1033631 | −0.4719368 |
| TRPV6 | | | | | | | | | |
| ADM)- | −0.2169028 | 0.12005403 | 0.15420623 | −0.088741 | −0.2593191 | 0.2410476 | 0.10417904 | −0.1515351 | 0.36720577 |
| CALCR | | | | | | | | | |
| ADM2)- | 0.0040195 | 0.12959974 | 0.16237064 | 0.17061924 | −0.1295192 | 0.13670653 | 0.11950433 | −0.2217025 | 0.03379558 |
| CALCR | | | | | | | | | |
| CALCB)- | 0.02846694 | −0.091586 | 0.12113921 | 0.12222845 | −0.2528679 | 0.02655684 | −0.3024092 | 0.10317225 | 0.04255599 |
| CALCR | | | | | | | | | |
| MBL2)- | −0.1359278 | −0.1443163 | 0.50318137 | −0.1037584 | −0.2097093 | 0.19343787 | 0.02916846 | −0.1727557 | 0.22432308 |
| CALCR | | | | | | | | | |
| GSTO1)- | −0.3183316 | −0.2446626 | 0.15352153 | 0.3321145 | 0.02713217 | 0.13407122 | 0.13594862 | −0.1426924 | −0.0374848 |
| RYR1 | | | | | | | | | |
| TNFSF13B)- | −0.0986556 | −0.0887703 | 0.02975681 | 0.2473731 | −0.292519 | −0.0100135 | 0.04167216 | 0.03583899 | 0.32452731 |
| TFRC | | | | | | | | | |
| B2M)- | 0.32900718 | −0.2274195 | 0.49295032 | −0.1192358 | 0.01844654 | 0.34940711 | −0.0581725 | 0.12806324 | 0.12476943 |
| TFRC | | | | | | | | | |
| VEGFC)- | 0.13206801 | −0.1489144 | 0.06069393 | 0.22128529 | −0.0941525 | −0.2773275 | 0.12926604 | −0.002306 | −0.0837945 |
| NRP2 | | | | | | | | | |
| SEMA3B)- | −0.0734078 | −0.2981948 | −0.0475092 | 0.07029217 | −0.008763 | 0.23664097 | −0.2462775 | −0.0535002 | 0.19367589 |
| NRP2 | | | | | | | | | |
| VEGFA)- | −0.2026025 | 0.30711462 | 0.05587034 | 0.28656126 | 0.05955989 | −0.0902652 | 0.00909241 | 0.15105093 | −0.3311045 |
| NRP2 | | | | | | | | | |
| PGF)- | −0.2780139 | −0.2851496 | −0.0585793 | 0.07351779 | −0.2088549 | 0.31171142 | −0.0428251 | 0.17512766 | 0.23175231 |
| NRP2 | | | | | | | | | |
| CXCL2)- | −0.1957858 | −0.0591269 | 0.0702765 | −0.1401845 | −0.1302657 | −0.1604137 | 0.09737287 | −0.0002635 | 0.28057578 |
| DPP4 | | | | | | | | | |
| ADCYAP1)- | −0.028594 | 0.11279112 | 0.17108953 | −0.0028988 | −0.1206198 | 0.00619317 | 0.04568529 | −0.0083007 | −0.0301713 |
| DPP4 | | | | | | | | | |
| GCG)- | −0.1179746 | 0.11293033 | −0.0676491 | −0.2088549 | −0.0423947 | 0.06140669 | 0.03401675 | −0.113706 | 0.14295125 |
| DPP4 | | | | | | | | | |
| CXCL9)- | −0.0455309 | 0.40706333 | 0.10401558 | −0.1351868 | −0.181105 | 0.22900814 | −0.1841256 | 0.10619586 | −0.2198946 |
| DPP4 | | | | | | | | | |
| CXCL12)- | 0.18796155 | −0.273 | −0.0140524 | 0.23565452 | 0.00527426 | 0.04809434 | 0.09367792 | −0.0596858 | 0.23675889 |
| DPP4 | | | | | | | | | |
| NPY)- | 0.27999475 | −0.1592856 | −0.0483743 | −0.0438105 | −0.3402559 | 0.3241427 | −0.0826686 | −0.0496723 | −0.3438735 |
| DPP4 | | | | | | | | | |
| ADA)- | −0.078518 | 0.05260731 | 0.09447157 | 0.24653928 | −0.1459859 | 0.14766475 | 0.20568947 | 0.01831478 | 0.03537666 |
| DPP4 | | | | | | | | | |
| MMP9)- | 0.13751071 | −0.1424524 | 0.27744085 | 0.08762972 | −0.2765418 | 0.19278538 | −0.2318086 | 0.25520422 | −0.0221344 |
| TLR9 | | | | | | | | | |
| HRAS)- | 0.28221264 | 0.11353079 | −9.89E−05 | 0.00224001 | 0.1340101 | −0.2979214 | 0.02952808 | −0.1277997 | 0.05704875 |
| TLR9 | | | | | | | | | |
| B2M)- | −0.1121945 | −0.1289281 | 0.07239212 | −0.0342556 | 0.06021836 | −0.0447958 | 0.12630686 | −0.0044796 | −0.0807642 |
| CD247 | | | | | | | | | |
| COL4A4)- | 0.02209473 | −0.0018467 | −0.0171817 | 0.11226477 | −0.1019843 | −0.1926234 | 0.17642796 | 0.1783985 | −0.0382753 |
| CD47 | | | | | | | | | |
| COL4A3)- | −0.1178139 | −0.023009 | −0.0795781 | −0.1699282 | 0.1537904 | 0.07175331 | 0.13953029 | 0.14440053 | −0.2530962 |
| CD47 | | | | | | | | | |
| THBS2)- | 0.33030303 | −0.058498 | 0.18604038 | 0.49789196 | −0.1311637 | −0.2379447 | 0.26364505 | 0.45125165 | −0.1629776 |
| CD47 | | | | | | | | | |
| CD55)- CR1 | −0.0243099 | 0.01594203 | −0.1261451 | −0.0089592 | −0.1310222 | 0.10448646 | −0.0492981 | 0.02964622 | 0.13675889 |
| C4B)- CR1 | 0.36338475 | 0.42570019 | 0.11095728 | −0.3148325 | 0.24221456 | −0.3143591 | 0.04514599 | 0.2965281 | 0.28748353 |
| C1QA)- | −0.2264717 | −0.1783985 | 0.16493658 | 0.22200264 | −0.0722995 | 0.09493379 | −0.0822514 | 0.07694842 | 0.23083004 |
| CR1 | | | | | | | | | |
| AFDN)- | 0.01212161 | 0.07595771 | 0.08966335 | 0.15217391 | 0.01752363 | −0.0806324 | −0.2930364 | −0.1283969 | 0.15270595 |
| NECTIN4 | | | | | | | | | |
| AFDN)- | −0.2246451 | 0.02961426 | −0.2594947 | 0.05659134 | −0.1498024 | −0.0491436 | −0.0697651 | −0.0729273 | 0.19178443 |
| F11R | | | | | | | | | |
| B2M)- | 0.03531194 | 0.03933065 | −0.2047868 | −0.0976285 | −0.1323181 | 0.10869565 | 0.05399527 | 0.04242424 | −0.0889328 |
| CD1B | | | | | | | | | |
| IL6)- IL6R | −0.2568366 | 0.06896327 | 0.27897397 | 0.06140467 | −0.2647117 | 0.01015295 | 0.02714723 | 0.18497647 | 0.15909615 |
| CD58)- | 0.12387573 | 0.13685633 | −0.1586182 | 0.12542819 | −0.0430398 | 0.28063241 | 0.34787772 | −0.2042161 | −0.3538867 |
| CD2 | | | | | | | | | |
| CD59)- | 0.01660079 | 0.02213439 | 0.0272212 | −0.0961792 | 0.20728977 | −0.0658762 | 0.06584499 | −0.1063241 | −0.1268775 |
| CD2 | | | | | | | | | |
| TNFSF15)- | −0.0642132 | 0.16822061 | 0.16529361 | −0.0653491 | −0.0113333 | −0.0866959 | 0.02925576 | 0.02167397 | 0.09776021 |
| TNFRSF6B | | | | | | | | | |
| TNFSF14)- | 0.01856249 | −0.1718375 | 0.2080201 | −0.0480253 | −0.2309897 | 0.25522103 | 0.00161439 | 0.10231241 | 0.15534109 |
| TNFRSF6B | | | | | | | | | |
| EDN1)- | 0.11511218 | 0.12288736 | 0.23532125 | −0.0117279 | −0.0983131 | 0.24154176 | −0.2515483 | 0.00125169 | −0.2789288 |
| ADGRL4 | | | | | | | | | |
| MDK)- | 0.31752306 | −0.3945982 | 0.43899868 | −0.5471673 | 0.3969697 | 0.41791831 | −0.6235383 | −0.611812 | 0.68968016 |
| TSPAN1 | | | | | | | | | |
| BMP7)- | −0.0545778 | −0.0575421 | 0.04442026 | 0.347047 | 0.10728413 | 0.05949008 | 0.01156889 | 0.11601555 | −0.0532956 |
| BMPR1A | | | | | | | | | |
| BMP2)- | −0.0433508 | 0.0192371 | 0.16925412 | −0.0652854 | −0.0079104 | 0.11430265 | −0.1636286 | 0.24075111 | 0.05916458 |
| BMPR1A | | | | | | | | | |
| TNFSF13B)- | −0.1986028 | 0.1823514 | 0.1083575 | −0.2505353 | −0.0155493 | 0.13202451 | 0.12953812 | −0.0548803 | −0.1747809 |
| CD40 | | | | | | | | | |
| LTB)- | 0.03811032 | 0.04654844 | −0.1616881 | −0.0421635 | 0.40936944 | 0.30833086 | 0.01699829 | 0.17325999 | 0.21054018 |
| CD40 | | | | | | | | | |
| ANGPTL2)- | −0.1769124 | 0.03472245 | 0.18424452 | −0.0680523 | −0.0302801 | 0.13650438 | 0.2204633 | −0.1471063 | −0.0613966 |
| TIE1 | | | | | | | | | |
| ANGPT1)- | 0.03472958 | 0.16002124 | −0.028997 | −0.1326174 | 0.07540579 | −0.1043581 | −0.0069266 | −0.2765572 | 0.08998682 |
| TIE1 | | | | | | | | | |
| NTF4)- | −0.0853196 | −0.154678 | 0.11625839 | 0.11812372 | −0.1342207 | 0.07385447 | −0.0736738 | 0.11576728 | 0.11279855 |
| BEX3 | | | | | | | | | |
| BDNF)- | 0.23007218 | 0.22512194 | −0.0808094 | 0.01515451 | 0.31889024 | −0.4069779 | 0.1662988 | −0.1248394 | 0.00131752 |
| BEX3 | | | | | | | | | |
| MDK)- | 0.18063241 | 0.00606061 | 0.19183136 | 0.32424242 | −0.01278 | −0.0171278 | −0.0183801 | 0.02905234 | −0.2325505 |
| SDC4 | | | | | | | | | |
| CXCL12)- | 0.00705409 | −0.1464878 | −0.01246 | 0.06594637 | 0.18334541 | 0.1172014 | 0.10974967 | 0.07114625 | −0.1288538 |
| SDC4 | | | | | | | | | |
| CCL5)- | 0.06364894 | 0.13171246 | 0.13349147 | −0.1889328 | −0.0301713 | 0.03504611 | 0.02832675 | 0.04611331 | −0.2544137 |
| SDC4 | | | | | | | | | |
| LAMA1)- | 0.44161836 | −0.3384291 | 0.38053507 | 0.04223496 | −0.343019 | −0.2951176 | 0.06943347 | 0.16416337 | −0.184585 |
| SDC4 | | | | | | | | | |
| BMP7)- | −0.1034312 | −0.0309132 | 0.00013192 | −0.0224645 | −0.0287428 | 0.01172789 | −0.2112203 | 0.29471257 | −0.0957212 |
| ENG | | | | | | | | | |
| BMP2)- | 0.12835211 | 0.05375848 | −0.1041962 | 0.00401858 | 0.10455537 | −0.1950255 | 0.16541951 | 0.04124934 | 0.00777441 |
| ENG | | | | | | | | | |
| CCN1)- | 0.21515152 | 0.0198946 | −0.3969164 | 0.12938076 | 0.23898004 | −0.0581028 | −0.442907 | 0.25704875 | 0.12476943 |
| TLR4 | | | | | | | | | |
| HSPA1A)- | 0.05006917 | −0.103103 | −0.0618411 | 0.21119895 | −0.0158793 | 0.20329381 | 0.15806813 | −0.1333333 | 0.23768116 |
| TLR4 | | | | | | | | | |
| IRAK4)- | −0.2897463 | −0.0846027 | 0.28456017 | 0.00639104 | −0.2740782 | 0.25162247 | −0.0039535 | −0.0306334 | −0.1984255 |
| TLR4 | | | | | | | | | |
| ZG16B)- | −0.0834816 | 0.00823615 | 0.04527481 | −0.2293808 | 0.09178362 | −0.1144928 | −0.3112151 | 0.23367811 | 0.3427762 |
| TLR4 | | | | | | | | | |
| NPY)- | 0.05365323 | −0.2551158 | 0.29417194 | 0.12609942 | 0.22174258 | 0.1088525 | −0.0704495 | −0.0537638 | 0.13768569 |
| NPY4R | | | | | | | | | |
| PPY)- | −0.1394291 | −0.2948389 | 0.02508984 | 0.17352922 | 0.13105085 | −0.1553667 | 0.27276923 | 0.06423983 | 0.13419414 |
| NPY4R | | | | | | | | | |
| ALOX5AP)- | −0.1905578 | −0.0878332 | 0.13415719 | 0.12450593 | −0.0034265 | 0.25876153 | −0.1186083 | 0.26561265 | 0.1944664 |
| ALOX5 | | | | | | | | | |
| ADIPOQ)- | 0.18552558 | 0.11160523 | 0.07745783 | 0.0344555 | −0.1495734 | 0.20086962 | 0.0413784 | 0.09091209 | 0.18979545 |
| CNR2 | | | | | | | | | |
| BMP7)- | −0.0955112 | 0.12022544 | 0.34902121 | 0.07595771 | −0.0475657 | −0.1320904 | −0.2396732 | −0.1081758 | −0.1280675 |
| BMPR2 | | | | | | | | | |
| BMP2)- | 0.04460256 | −0.3707095 | −0.1043252 | 0.06409961 | −0.013242 | −0.2348969 | −0.194169 | 0.1074617 | −0.0250362 |
| BMPR2 | | | | | | | | | |
| RGMA)- | −0.1095259 | −0.1926853 | 0.02336156 | 0.13948279 | −0.0575212 | 0.13217369 | 0.06890873 | −0.246319 | −0.0591568 |
| BMPR2 | | | | | | | | | |
| VEGFA)- | −0.0579748 | 0.2083004 | 0.01680229 | 0.14664032 | 0.0776859 | 0.00092233 | 0.08121376 | 0.08011332 | −0.1135084 |
| EPHB2 | | | | | | | | | |
| AFDN)- | 0.05310143 | −0.2292566 | 0.04586189 | −0.1918314 | −0.0681317 | 0.05283616 | −0.1462836 | 0.15073953 | −0.1994796 |
| EPHB2 | | | | | | | | | |
| PLTP)- | −0.0358449 | 0.22369375 | 0.16357286 | −0.1839982 | −0.006857 | −0.0272745 | 0.16568873 | −0.1768232 | 0.0881452 |
| ABCA1 | | | | | | | | | |
| HLA-C)- | 0.32159216 | 0.40106723 | −0.1381787 | 0.06837945 | −0.1218331 | 0.14971994 | −0.0170659 | −0.0055354 | 0.01844532 |
| NOTCH4 | | | | | | | | | |
| LTA)- | 0.16237149 | 0.15139234 | −0.1579069 | −0.005337 | 0.02172265 | 0.01950384 | 0.08693934 | −0.0181165 | 0.03847167 |
| TNFRSF1B | | | | | | | | | |
| TNF)- | −0.1213121 | −0.1321203 | 0.18701145 | 0.27862534 | −0.2644402 | 0.28865166 | −0.008305 | 0.20830726 | 0.09011858 |
| TNFRSF1B | | | | | | | | | |
| GDNF)- | −0.2137656 | −0.0509999 | 0.29916304 | 0.2051451 | −0.0436845 | 0.21062687 | −0.0177571 | 0.07777448 | 0.19223295 |
| EDNRB | | | | | | | | | |
| EDN1)- | 0.13837638 | 0.1460763 | 0.04438367 | 0.34273291 | 0.17047214 | 0.01554985 | −0.1036403 | −0.0924303 | −0.445191 |
| EDNRB | | | | | | | | | |
| NPNT)- | 0.30516571 | −0.1130621 | −0.2373039 | −0.2186502 | −0.1839226 | 0.46162461 | −0.2328543 | 0.05830424 | −0.0415033 |
| ITGA8 | | | | | | | | | |
| NRG1)- | −0.0186602 | −0.1212581 | 0.21680797 | 0.03023915 | 0.06235794 | 0.07167798 | 0.17299648 | 0.09710145 | −0.1046113 |
| LGR4 | | | | | | | | | |
| TNFSF11)- | −0.0742742 | −0.3557518 | 0.18546103 | 0.00639146 | −0.0564049 | −0.0706355 | 0.1060641 | 0.31119895 | −0.3516469 |
| LGR4 | | | | | | | | | |
| COL8A2)- | −0.0693246 | −0.1436573 | −0.0074477 | 0.36160611 | −0.1041049 | −0.1406502 | −0.3289738 | 0.03610013 | −0.0450593 |
| SLC4A11 | | | | | | | | | |
| NMB)- | −0.1850729 | −0.2237021 | −0.0650884 | 0.27155045 | 0.12427518 | 0.44528625 | −0.0425013 | 0.11021806 | 0.40864324 |
| GRPR | | | | | | | | | |
| SPTBN2)- | 0.10804666 | −0.1164415 | −0.073481 | −0.1293808 | −0.1963239 | 0.20627182 | 0.18578299 | −0.1994861 | 0.36640316 |
| PTPRA | | | | | | | | | |
| NCAM1)- | 0.01699885 | −0.0327437 | −0.1919618 | −0.0502009 | 0.14402425 | −0.148142 | 0.00059292 | 0.07049213 | 0.00263505 |
| PTPRA | | | | | | | | | |
| IRAK4)- | −0.0834158 | −0.1321465 | 0.07415472 | −0.0223357 | −0.0925511 | 0.00322846 | −0.0572766 | 0.15797622 | 0.1823512 |
| TLR7 | | | | | | | | | |
| MDK)- | 0.22239789 | −0.0378129 | 0.29565217 | −0.3488801 | 0.14545455 | 0.42160738 | −0.4509371 | −0.3434896 | 0.04703712 |
| SDC1 | | | | | | | | | |
| CCL5)- | 0.31303947 | 0.12209264 | 0.14502208 | −0.002108 | −0.0744401 | −0.3002635 | −0.1433465 | −0.3884058 | 0.04940711 |
| SDC1 | | | | | | | | | |
| SLIT2)- | −0.0423612 | 0.27379933 | 0.11443442 | −0.0214756 | −0.5945982 | −0.4032938 | 0.23412385 | 0.19354414 | 0.21067194 |
| SDC1 | | | | | | | | | |
| IL6)- IL6ST | −0.0890721 | 0.0690292 | −0.1459797 | 0.19370141 | 0.08756671 | 0.20740546 | −0.2584577 | −0.0006588 | −0.3456636 |
| NPS)- | −0.1158026 | −0.0421165 | 0.06682525 | −0.2111989 | 0.1360746 | 0.11839505 | 0.13264802 | −0.113256 | 0.39196311 |
| NPSR1 | | | | | | | | | |
| B2M)- | −0.3616299 | −0.2677383 | 0.43397128 | 0.31554677 | −0.284678 | 0.11838335 | −0.2629116 | 0.14835798 | 0.27786561 |
| KLRC2 | | | | | | | | | |
| HLA-G)- | −0.4376441 | −0.5510244 | 0.51195731 | −0.2129117 | 0.05375494 | −0.0927536 | 0.11422925 | −0.2480896 | −0.1965744 |
| KLRD1 | | | | | | | | | |
| B2M)- | −0.3249226 | −0.3802622 | 0.3097042 | −0.2194993 | 0.14993412 | −0.2666667 | 0.13306983 | −0.1998682 | −0.1353096 |
| KLRD1 | | | | | | | | | |
| HLA-B)- | −0.5849336 | −0.724398 | 0.44909253 | −0.3815547 | 0.30816864 | −0.2719368 | 0.15309618 | −0.1034256 | −0.0490119 |
| KLRD1 | | | | | | | | | |
| HLA-G)- | 0.22166952 | 0.32854602 | 0.14824212 | 0.12134387 | −0.1049059 | 0.07391791 | 0.23076671 | −0.2551551 | 0.14953887 |
| KIR3DL1 | | | | | | | | | |
| B2M)- | −0.1137831 | 0.34060215 | 0.15400837 | 0.21396574 | 0.13798557 | −0.049608 | 0.18048829 | −0.0664734 | 0.07957839 |
| KIR3DL1 | | | | | | | | | |
| HLA-B)- | 0.10053694 | 0.20554037 | 0.20095553 | 0.28537549 | 0.17139469 | −0.1100863 | 0.10602617 | −0.2266948 | 0.32226614 |
| KIR3DL1 | | | | | | | | | |
| HLA-G)- | −0.0724709 | −0.064497 | 0.21750017 | −0.1140975 | −0.1308473 | −0.0393294 | −0.1758466 | −0.0893969 | −0.0935441 |
| LILRB2 | | | | | | | | | |
| B2M)- | −0.0154824 | −0.1390737 | 0.17707716 | 0.18050066 | −0.2683489 | −0.0174578 | −0.1320332 | −0.1275404 | 0.06152833 |
| LILRB2 | | | | | | | | | |
| INS)- | −0.0225992 | −0.0942153 | 0.01067475 | 0.01574492 | 0.20247076 | −0.030865 | −0.112729 | 0.00408446 | −0.0783926 |
| LILRB2 | | | | | | | | | |
| HLA-B)- | −0.0593583 | −0.0085642 | 0.11688355 | 0.09341238 | −0.2884438 | −0.0234527 | −0.1383582 | −0.1576468 | −0.1163373 |
| LILRB2 | | | | | | | | | |
| MDK)- | 0.25115299 | 0.06008103 | −0.0979488 | 0.03353207 | −0.025526 | 0.37264462 | 0.01879887 | −0.0905946 | −0.3188286 |
| PTPRZ1 | | | | | | | | | |
| CCL5)- | −0.063058 | 0.32582197 | −0.028837 | −0.2413702 | 0.08771295 | 0.04888172 | 0.12171736 | 0.0863006 | −0.1507246 |
| GPR75 | | | | | | | | | |
| TNF)- VSIR | −0.2100545 | −0.1032471 | 0.06703924 | 0.40022409 | −0.0810867 | 0.21128247 | −0.0320398 | 0.11581409 | 0.00625844 |
| VEGFC)- | −0.1143874 | −0.1972787 | −0.1804105 | 0.11133437 | 0.03445436 | 0.02911822 | −0.0542819 | 0.25599473 | −0.1163373 |
| ITGB1 | | | | | | | | | |
| LAMA2)- | 0.09526319 | 0.00026352 | 0.11265564 | 0.15928854 | 0.08445323 | −0.1457181 | 0.19986825 | 0.05046113 | 0.09868248 |
| ITGB1 | | | | | | | | | |
| LAMB2)- | −0.0811594 | 0.04268775 | 0.10619236 | −0.2086504 | −0.2112198 | 0.2994367 | −0.0028986 | 0.08274045 | −0.071278 |
| ITGB1 | | | | | | | | | |
| NPNT)- | −0.1724206 | 0.07767822 | 0.0747134 | 0.04848485 | 0.06653491 | −0.2919631 | −0.1841897 | −0.3546772 | −0.2088274 |
| ITGB1 | | | | | | | | | |
| DSPP)- | 0.09800627 | 0.09932445 | 0.15145825 | −0.1543783 | −0.1338209 | 0.14515385 | −0.0494071 | −0.0478261 | −0.1031621 |
| ITGB1 | | | | | | | | | |
| MDK)- | 0.08722003 | 0.34716733 | 0.28708827 | 0.43254282 | 0.36982872 | 0.11080369 | −0.4057446 | −0.1125202 | −0.3967193 |
| ITGB1 | | | | | | | | | |
| COL4A4)- | −0.066482 | −0.189487 | −0.1844085 | −0.1626949 | −0.1876793 | 0.14555526 | 0.19150828 | 0.06976514 | 0.17457756 |
| ITGB1 | | | | | | | | | |
| COL4A3)- | −0.0391614 | 0.04654537 | 0.02742617 | 0.0620676 | 0.075509 | −0.0247084 | −0.0932806 | −0.3754941 | −0.0700922 |
| ITGB1 | | | | | | | | | |
| ADAM9)- | −0.185112 | 0.06482213 | 0.09710145 | 0.10158103 | 0.09960474 | −0.2080369 | −0.1119895 | 0.39262187 | −0.1359684 |
| ITGB1 | | | | | | | | | |
| ANGPT1)- | −0.1089015 | −0.0602883 | −0.0339699 | −0.2093682 | −0.1763621 | −0.1129192 | 0.00342556 | −0.1942029 | −0.0156785 |
| ITGB1 | | | | | | | | | |
| VEGFA)- | −0.2492754 | 0.55744401 | 0.50619236 | 0.53860343 | 0.42345191 | −0.3408432 | −0.3176653 | 0.26878356 | −0.4712935 |
| ITGB1 | | | | | | | | | |
| SEMA4D)- | 0.01877965 | 0.02141539 | 0.18600975 | 0.15072464 | −0.0376538 | −0.0837945 | 0.03007025 | −0.0613966 | 0.07364954 |
| CD72 | | | | | | | | | |
| MDK)- | 0.1627141 | 0.08722003 | −0.0722336 | 0.18181818 | −0.0995848 | 0.23794466 | 0.12094251 | −0.3239237 | −0.3341349 |
| ITGA4 | | | | | | | | | |
| IL6)- HRH1 | −0.0519566 | 0.21414215 | 0.03560597 | −0.212808 | 0.12861991 | −0.0566647 | 0.13779813 | 0.12448529 | 0.1145624 |
| CCN1)- | 0.44242424 | 0.43820817 | −0.476658 | −0.0217391 | 0.20742596 | −0.0039526 | −0.4567588 | 0.46034256 | 0.48379447 |
| ITGB2 | | | | | | | | | |
| SPON2)- | 0.33992095 | 0.32094862 | −0.3296544 | −0.4689065 | 0.37867757 | −0.4296443 | −0.5472276 | 0.57338603 | 0.55783926 |
| ITGB2 | | | | | | | | | |
| ICAM2)- | 0.2815027 | 0.1513044 | −0.2561685 | −0.1731453 | −0.0259646 | −0.0923705 | 0.1005502 | −0.0256917 | −0.0844532 |
| ITGB2 | | | | | | | | | |
| TNF)- | −0.0336916 | 0.04364453 | 0.09543282 | −0.0863949 | −0.0285451 | 0.057205 | 0.15420608 | 0.16799526 | 0.16785244 |
| TRPM2 | | | | | | | | | |
| VEGFA)- | −0.0021082 | −0.1812912 | −0.01272 | −0.2007905 | −0.1024188 | 0.09012451 | 0.13702423 | −0.183022 | 0.14750156 |
| SIRPA | | | | | | | | | |
| GCG)- | 0.14264537 | 0.0200962 | −0.0785161 | −0.0566628 | −0.100376 | 0.01291343 | 0.04622488 | 0.09591568 | −0.0619915 |
| GCGR | | | | | | | | | |
| IFNE)- | 0.14087525 | 0.06119221 | −0.0793672 | 0.29361968 | −0.2156695 | 0.20304368 | 0.05330961 | −0.3242086 | −0.2751828 |
| ADGRV1 | | | | | | | | | |
| FSHB)- | −0.0350351 | 0.039602 | 0.10957324 | 0.13796739 | −0.0286675 | 0.08522278 | 0.00329457 | 0.12969127 | 0.21054018 |
| FSHR | | | | | | | | | |
| SYTL3)- | −0.126619 | −0.1692592 | 0.04728616 | 0.26542376 | 0.03829802 | 0.17912906 | −0.0762005 | 0.19302984 | 0.23650318 |
| NRXN1 | | | | | | | | | |
| LAMA2)- | 0.11884841 | −0.1390078 | 0.06976744 | 0.19907773 | −0.1115942 | −0.2501976 | 0.10790514 | 0.20685112 | −0.2857708 |
| ITGA6 | | | | | | | | | |
| ADAM9)- | 0.30645586 | −0.5173913 | 0.29986825 | −0.545191 | 0.28774704 | 0.3002635 | −0.330303 | −0.2803689 | 0.62753623 |
| ITGA6 | | | | | | | | | |
| ADM)- | −0.1308645 | 0.0190426 | 0.15271553 | 0.06199354 | 0.04270322 | 0.02384952 | 0.19341022 | 0.11647671 | 0.06614184 |
| CALCRL | | | | | | | | | |
| CALCB)- | −0.0520542 | −0.052909 | 0.06169056 | 0.10134089 | −0.076193 | 0.33249869 | 0.11597641 | 0.03504727 | −0.1487484 |
| CALCRL | | | | | | | | | |
| IRAK4)- | −0.0447756 | 0.13630525 | 0.14217386 | 0.06312183 | 0.05349374 | −0.1200501 | 0.08752967 | −0.1195072 | −0.1407207 |
| TLR6 | | | | | | | | | |
| FGF21)- | 0.07132029 | −0.088782 | −0.092617 | 0.24057971 | 0.15232574 | −0.2936294 | 0.2925383 | −0.2275587 | 0.36845746 |
| FGFR1 | | | | | | | | | |
| NCAM1)- | 0.04984352 | 0.14599598 | 0.1240734 | 0.06634166 | 0.09257429 | −0.0643036 | 0.07893003 | 0.1469135 | 0.1057971 |
| FGFR1 | | | | | | | | | |
| FGF12)- | 0.19429891 | 0.03031601 | 0.05154402 | −0.2218855 | −0.132569 | −0.1881012 | 0.02674924 | 0.01370314 | −0.0121212 |
| FGFR1 | | | | | | | | | |
| PRG4)- | 0.16913751 | −0.0195032 | 0.04118073 | 0.02621957 | −0.1344577 | −0.0274054 | 0.09459816 | −0.1469038 | −0.0353096 |
| CD44 | | | | | | | | | |
| VIM)- | −0.1890646 | 0.15217391 | −0.2100132 | 0.17048748 | 0.16284585 | −0.0059289 | −0.1295125 | −0.1072464 | 0.11027668 |
| CD44 | | | | | | | | | |
| RGMA)- | −0.210835 | −0.0836244 | −0.010518 | 0.09731511 | −0.0464166 | 0.24631808 | 0.12380108 | −0.1305837 | −0.2873518 |
| BMPR1B | | | | | | | | | |
| SEMA4D)- | −0.5582499 | 0.45288615 | −0.5379547 | −0.386693 | 0.52714097 | 0.54426877 | 0.55230567 | 0.48919631 | −0.2667984 |
| PLXNB2 | | | | | | | | | |
| FGF21)- | −0.1092216 | −0.2573161 | 0.05533925 | −0.1379447 | −0.1568603 | 0.05527373 | −0.2395859 | −0.0148895 | −0.0246385 |
| FGFR2 | | | | | | | | | |
| NCAM1)- | −0.1826717 | 0.03946372 | 0.26048111 | −0.0457211 | −0.2880139 | 0.10884174 | −0.2068603 | −0.0324791 | −0.0104084 |
| FGFR2 | | | | | | | | | |
| FGF12)- | −0.4622179 | 0.10188817 | 0.11251531 | −0.1555438 | −0.0836467 | 0.48158519 | 0.06484175 | 0.27452401 | −0.0640316 |
| FGFR2 | | | | | | | | | |
| CXCL17)- | 0.21173373 | −0.2938133 | 0.38873314 | −0.3421061 | 0.29565912 | 0.25638999 | −0.162629 | −0.1192358 | 0.27418558 |
| GPR35 | | | | | | | | | |
| B2M)- HFE | 0.06919503 | 0.0290533 | −0.0666161 | 0.39841897 | 0.0858698 | −0.0876413 | 0.1641551 | −0.1069487 | 0.27588933 |
| RGMA)- | −0.0771893 | 0.26154861 | 0.11994066 | −0.2777137 | −0.2286281 | 0.07157451 | 0.05348726 | −0.1380515 | 0.15507246 |
| HFE | | | | | | | | | |
| NCAM1)- | 0.08759389 | −0.2989096 | −0.0866113 | 0.23242638 | −0.2556086 | 0.2637283 | 0.17201963 | −0.0077736 | 0.23096179 |
| ROBO1 | | | | | | | | | |
| SLIT2)- | −0.0206213 | 0.16147309 | −0.0893098 | −0.1731225 | −0.1051487 | −0.1077769 | −0.1310406 | 0.08333608 | 0.21304348 |
| ROBO1 | | | | | | | | | |
| RGMA)- | −0.1912748 | −0.0628007 | 0.49533543 | −0.0132433 | −0.3900462 | 0.36387297 | 0.39182737 | −0.3463882 | 0.07325428 |
| TFR2 | | | | | | | | | |
| B2M)- | −0.3896831 | −0.4672903 | 0.45836521 | 0.04255599 | −0.155977 | 0.371278 | −0.20432 | 0.3715415 | 0.12213439 |
| CD3G | | | | | | | | | |
| CCL5)- | −0.0991632 | 0.04843493 | 0.14875244 | 0.00830149 | −0.1415719 | 0.12411067 | −0.1103971 | 0.06192358 | 0.17604428 |
| CCR3 | | | | | | | | | |
| CCL14)- | −0.0641038 | 0.03235265 | −0.1134401 | 0.02147916 | −0.0402386 | 0.0857077 | 0.24761908 | −0.1678524 | 0.03149295 |
| CCR3 | | | | | | | | | |
| CCL15)- | 0.12696995 | −0.1648448 | −0.2373106 | 0.02556416 | 0.02949513 | 0.20283936 | 0.00718405 | −0.2747036 | 0.06707076 |
| CCR3 | | | | | | | | | |
| CCL18)- | −0.064582 | −0.1538309 | 0.20795148 | 0.24628562 | 0.00751384 | 0.13327185 | −0.1262811 | −0.041502 | 0.28330479 |
| CCR3 | | | | | | | | | |
| SPON2)- | 0.27299078 | −0.0629776 | −0.2086601 | −0.2604743 | 0.27377582 | −0.3129117 | −0.2903184 | 0.33952569 | 0.22332016 |
| ITGAM | | | | | | | | | |
| ADCYAP1)- | −0.1146358 | −0.0251672 | 0.1631631 | 0.14711599 | −0.0056013 | 0.16655137 | −0.0637828 | 0.08722003 | 0.43583663 |
| GPR84 | | | | | | | | | |
| VEGFA)- | 0.02477351 | 0.22307135 | 0.10982278 | 0.03524606 | 0.14355359 | 0.04836106 | 0.16859373 | −0.1794821 | −0.1394077 |
| ITGB3 | | | | | | | | | |
| VEGFA)- | 0.21963833 | 0.26616596 | −0.1450412 | 0.31603914 | −0.041318 | 0.14190191 | −0.0249761 | −0.0492786 | −0.2306957 |
| GRIN2B | | | | | | | | | |

### Data Availability

Raw data will be available in the controlled access repository Data Use Oversight System (DUOS) at the Broad Institute: https://duos.broadinstitute.org/under its Data Access Committee. Processed annotated datasets will be provided in the Single Cell Portal upon publication.

### Code Availability

All code will be available upon publication in Github at https://github.com/karthikj89/humanpdac.

### REFERENCES FOR EXAMPLE 1

1 Versteijne, E. et al. Preoperative Chemoradiotherapy Versus Immediate Surgery for Resectable and Borderline Resectable Pancreatic Cancer: Results of the Dutch Randomized Phase III PREOPANC Trial. J. Clin. Oncol. 38, 1763-1773 (2020).
2. Mizrahi, J. D., Surana, R., Valle, J. W. & Schroff, R. T. Pancreatic cancer. Lancet 395, 2008-2020 (2020).
3. Siegel, R. L., Miller, K. D., Fuchs, H. E. & Jemal, A. Cancer Statistics, 2021. CA. Cancer J. Clin. 71, 7-33 (2021).
4. Koucký, V., Bouček, J. & Fialová, A. Immunology of plasmacytoid dendritic cells in solid tumors: A brief review. Cancers vol. 11 (2019).
5. Murphy, J. E. et al. Total Neoadjuvant Therapy with FOLFIRINOX in Combination with Losartan Followed by Chemoradiotherapy for Locally Advanced Pancreatic Cancer: A Phase 2 Clinical Trial. JAMA Oncol. 5, 1020-1027 (2019).
6. Collisson, E. A., Bailey, P., Chang, D. K. & Biankin, A. V. Molecular subtypes of pancreatic cancer. Nat. Rev. Gastroenterol. Hepatol. 16, 207-220 (2019).
7. Guo, J. A. et al. Refining the Molecular Framework for Pancreatic Cancer with Single-cell and Spatial Technologies. Clin. Cancer Res. 27, 3825-3833 (2021).
8. Collisson, E. A. et al. Subtypes of pancreatic ductal adenocarcinoma and their differing responses to therapy. Nat. Med. 17, 500-503 (2011).
9. Moffitt, R. A. et al. Virtual microdissection identifies distinct tumor- and stroma-specific subtypes of pancreatic ductal adenocarcinoma. Nat. Genet. 47, 1168-1178 (2015).
10. Bailey, P. et al. Genomic analyses identify molecular subtypes of pancreatic cancer. Nature 531, 47-52 (2016).
11. Raphael, B. J. et al. Integrated Genomic Characterization of Pancreatic Ductal Adenocarcinoma. Cancer Cell 32, 185-203 (2017).
12. Sivakumar, S., de Santiago, I., Chlon, L. & Markowetz, F. Master Regulators of Oncogenic KRAS Response in Pancreatic Cancer: An Integrative Network Biology Analysis. PLOS Med. 14, (2017).
13. de Santiago, I. et al. Immuno-phenotypes of Pancreatic Ductal Adenocarcinoma: Metaanalysis of transcriptional subtypes. bioRxiv (2017) doi: 10.1101/198903.
14. Aung, K. L. et al. Genomics-driven precision medicine for advanced pancreatic cancer: Early results from the COMPASS trial. Clin. Cancer Res. 24, 1344-1354 (2018).
15. Kroemer, G., Galluzzi, L., Kepp, O. & Zitvogel, L. Immunogenic cell death in cancer therapy. Anm. Rev. Immunol. 31, 51-72 (2013).
16. Coffelt, S. B. & de Visser, K. E. Immune-mediated mechanisms influencing the efficacy of anticancer therapies. Trends Immunol. 36, 198-216 (2015).
17. Hosein, A. N., Brekken, R. A. & Maitra, A. Pancreatic cancer stroma: an update on therapeutic targeting strategies. Nat. Rev. Gastroenterol. Hepatol. 17, 487-505 (2020).
18. Chauhan, V. P. et al. Angiotensin inhibition enhances drug delivery and potentiates chemotherapy by decompressing tumor blood vessels. Nat. Commun. 4, 2516 (2013).
19. Barker, H. E., Paget, J. T. E., Khan, A. A. & Harrington, K. J. The tumor microenvironment after radiotherapy: Mechanisms of resistance and recurrence. Nat. Rev. Cancer 15, 409-25 (2015).
20. Liu, H. et al. Use of angiotensin system inhibitors is associated with immune activation and longer survival in nonmetastatic pancreatic ductal adenocarcinoma. Clin. Cancer Res. 23, (2017).
21. Sørensen, B. S. & Horsman, M. R. Tumor Hypoxia: Impact on Radiation Therapy and Molecular Pathways. Front. Oncol. 10, 562 (2020).
22. Patel, A. P. et al. Single-cell RNA-seq highlights intratumoral heterogeneity in primary glioblastoma. Science (80-.). 344, 1396-1401 (2014).
23. Tirosh, I. et al. Single-cell RNA-seq supports a developmental hierarchy in human oligodendroglioma. Nature 539, 309-313 (2016).
24. Venteicher, A. S. et al. Decoupling genetics, lineages, and microenvironment in IDH-mutant gliomas by single-cell RNA-seq. Science (80-.). 355, (2017).
25. Puram, S. V et al. Single-Cell Transcriptomic Analysis of Primary and Metastatic Tumor Ecosystems in Head and Neck Cancer. Cell 171, 1611-1624 (2017).
26. Steele, N. G. et al. Multimodal Mapping of the Tumor and Peripheral Blood Immune Landscape in Human Pancreatic Cancer. Nat. Cancer 1, 1097-1112 (2020).
27 Azevedo-Pouly, A. C. P., Elgamal, O. A. & Schmittgen, T. D. RNA isolation from mouse pancreas: A ribonuclease-rich tissue. J. Vis. Exp. e51779 (2014) doi: 10.3791/51779.
28. Augereau, C., Lemaigre, F. P. & Jacquemin, P. Extraction of high-quality RNA from pancreatic tissues for gene expression studies. Anal. Biochem. 500, 60-62 (2016).
29. Jun, E. et al. Method Optimization for Extracting High-Quality RNA From the Human Pancreas Tissue. Transl. Oncol. 11, 800-807 (2018).
30. Von Ahrens, D., Bhagat, T. D., Nagrath, D., Maitra, A. & Verma, A. The role of stromal cancer-associated fibroblasts in pancreatic cancer. Journal of Hematology and Oncology vol. 10 (2017).
31. Habib, N. et al. Div-Seq: Single-nucleus RNA-Seq reveals dynamics of rare adult newborn neurons. Science (80-.). 353, 925-928 (2016).
32. Habib, N. et al. Massively parallel single-nucleus RNA-seq with DroNc-seq. Nat. Methods 14, 955-958 (2017).
33. Drokhlyansky, E. et al. The Human and Mouse Enteric Nervous System at Single-Cell Resolution. Cell 182, 1606-1622.e23 (2020).
34. Tosti, L. et al. Single-Nucleus and In Situ RNA-Sequencing Reveal Cell Topographies in the Human Pancreas. Gastroenterology 160, 1330-1344 (2021).
35. Barthelson, R. A., Lambert, G. M., Vanier, C., Lynch, R. M. & Galbraith, D. W. Comparison of the contributions of the nuclear and cytoplasmic compartments to global gene expression in human cells. BMC Genomics 8, 340 (2007).
36. Abdelmoez, M. N. et al. SINC-seq: Correlation of transient gene expressions between nucleus and cytoplasm reflects single-cell physiology. Genome Biol. 19, (2018).
37. Slyper, M. et al. A single-cell and single-nucleus RNA-Seq toolbox for fresh and frozen human tumors. Nat. Med. 26, 792-802 (2020).
38. Han, J., DePinho, R. A. & Maitra, A. Single-cell RNA sequencing in pancreatic cancer. Nat. Rev. Gastroenterol. Hepatol. 18, 451-452 (2021).
39. Ligorio, M. et al. Stromal Microenvironment Shapes the Intratumoral Architecture of Pancreatic Cancer. Cell 178, 160-175 (2019).
40. Väyrynen, S. A. et al. Composition, Spatial Characteristics, and Prognostic Significance of Myeloid Cell Infiltration in Pancreatic Cancer. Clin. Cancer Res. 27, 1069-1081 (2021).
41. Liudahl, S. M. et al. Leukocyte Heterogeneity in Pancreatic Ductal Adenocarcinoma: Phenotypic and Spatial Features Associated with Clinical Outcome. Cancer Discov. 11, 2014-2031 (2021).
42. Grünwald, B. T. et al. Spatially confined sub-tumor microenvironments in pancreatic cancer. Cell 184, 1-16 (2021).
43. Merritt, C. R. et al. Multiplex digital spatial profiling of proteins and RNA in fixed tissue. Nat. Biotechnol. 38, 586-599 (2020).
44. Gentles, A. J. et al. The prognostic landscape of genes and infiltrating immune cells across human cancers. Nat. Med. 21, 938-945 (2015).
45. Muraro, M. J. et al. A Single-Cell Transcriptome Atlas of the Human Pancreas. Cell Syst. 3, 385-394 (2016).
46. Zilionis, R. et al. Single-Cell Transcriptomics of Human and Mouse Lung Cancers Reveals Conserved Myeloid Populations across Individuals and Species. Immunity 50, 1317-1334 (2019).
47. Schupp, J. C. et al. Integrated Single Cell Atlas of Endothelial Cells of the Human Lung. Circulation 144, 286-302 (2021).
48. Bernard, V. et al. Single-cell transcriptomics of pancreatic cancer precursors demonstrates epithelial and microenvironmental heterogeneity as an early event in neoplastic progression. Clin. Cancer Res. 25, 2194-2205 (2019).
49. Elyada, E. et al. Cross-species single-cell analysis of pancreatic ductal adenocarcinoma reveals antigen-presenting cancer-associated fibroblasts. Cancer Discov. 9, 1102-1123 (2019).
50. Moncada, R. et al. Integrating microarray-based spatial transcriptomics and single-cell RNA-seq reveals tissue architecture in pancreatic ductal adenocarcinomas. Nat. Biotechnol. 38, 333-342 (2020).
51. Peng, J. et al. Single-cell RNA-seq highlights intra-tumoral heterogeneity and malignant progression in pancreatic ductal adenocarcinoma. Cell Res. 29, 725-738 (2019).
52. Wherry, E. J. et al. Molecular Signature of CD8+ T Cell Exhaustion during Chronic Viral Infection. Immunity 27, 670-684 (2007).
53. Eberlein, J. et al. Chemokine Signatures of Pathogen-Specific T Cells I: Effector T Cells. J. Immunol. 205, 2169-2187 (2020).
54. Johnston, R. J. et al. The Immunoreceptor TIGIT Regulates Antitumor and Antiviral CD8+ T Cell Effector Function. Cancer Cell 26, 923-937 (2014).
55. Chauvin, J.-M. et al. TIGIT and PD-1 impair tumor antigen-specific CD8+ T cells in melanoma patients. J. Clin. Invest. 125, 2046-2058 (2015).
56. Zhou, X. et al. Differentiation and Persistence of Memory CD8+ T Cells Depend on T Cell Factor 1. Immunity 33, 229-240 (2010).
57. Im, S. J. et al. Defining CD8+ T cells that provide the proliferative burst after PD-1 therapy. Nature 537, 417-421 (2016).
58. Siddiqui, I. et al. Intratumoral Tcf1+PD-1+CD8+ T Cells with Stem-like Properties Promote Tumor Control in Response to Vaccination and Checkpoint Blockade Immunotherapy. Immunity 50, 195-211.e10 (2019).
59. Carlson, C. M. et al. Kruppel-like factor 2 regulates thymocyte and T-cell migration. Nature 442, 299-302 (2006).
60. Willinger, T. et al. Human Naive CD8 T Cells Down-Regulate Expression of the WNT Pathway Transcription Factors Lymphoid Enhancer Binding Factor 1 and Transcription Factor 7 (T Cell Factor-1) following Antigen Encounter In Vitro and In Vivo. J. Immunol. 176, 1439-1446 (2006).
61. Incio, J. et al. Obesity-Induced Inflammation and Desmoplasia Promote Pancreatic Cancer Progression and Resistance to Chemotherapy. Cancer Discov. 6, 852-869 (2016).
62. De La O, J. P. et al. Notch and Kras reprogram pancreatic acinar cells to ductal intraepithelial neoplasia. Proc. Natl. Acad. Sci. U.S.A 105, (2008).
63. Guerra, C. et al. Chronic Pancreatitis Is Essential for Induction of Pancreatic Ductal Adenocarcinoma by K-Ras Oncogenes in Adult Mice. Cancer Cell 11, (2007).
64. Morris I V, J. P., Cano, D. A., Sekine, S., Wang, S. C. & Hebrok, M. β-catenin blocks Kras-dependent reprogramming of acini into pancreatic cancer precursor lesions in mice. J. Clin. Invest. 120, (2010).
65. Liberzon, A. et al. The Molecular Signatures Database Hallmark Gene Set Collection. Cell Syst. 1, 417-425 (2015).
66. Roa-Peña, L. et al. Keratin 17 identifies the most lethal molecular subtype of pancreatic cancer. Sci. Rep. 9, (2019).
67. Wolf, F. A. et al. PAGA: graph abstraction reconciles clustering with trajectory inference through a topology preserving map of single cells. Genome Biol. 20, 59 (2019).
68. Juiz, N. et al. Basal-like and classical cells coexist in pancreatic cancer revealed by single-cell analysis on biopsy-derived pancreatic cancer organoids from the classical subtype. FASEB J. 34, 12214-12228 (2020).
69. Chan-Seng-Yue, M. et al. Transcription phenotypes of pancreatic cancer are driven by genomic events during tumor evolution. Nat. Genet. 52, 231-240 (2020).
70. Raghavan, S. et al. The tumor microenvironment drives transcriptional phenotypes and their plasticity in metastatic pancreatic cancer. bioRxiv 6, 2020.08.25.256214 (2021).
71. Öhlund, D. et al. Distinct populations of inflammatory fibroblasts and myofibroblasts in pancreatic cancer. J. Exp. Med. 214, 579-596 (2017).
72. Buechler, M. B. et al. Cross-tissue organization of the fibroblast lineage. Nature 593, 575-579 (2021).
73. Kotliar, D. et al. Identifying gene expression programs of cell-type identity and cellular activity with. Elife 8, e43803 (2019).
74. Dardenne, E. et al. N-Myc Induces an EZH2-Mediated Transcriptional Program Driving Neuroendocrine Prostate Cancer. Cancer Cell 30, (2016).
75. Da Costa, J. B. et al. Molecular characterization of neuroendocrinelike bladder cancer. Clin. Cancer Res. 25, 3908-3920 (2019).
76. Qiu, H. et al. MEN1 deficiency leads to neuroendocrine differentiation of lung cancer and disrupts the DNA damage response. Nat. Commun. 11, 1009 (2020).
77. He, H. et al. Krüppel-like Factor 4 Promotes Esophageal Squamous Cell Carcinoma Differentiation by Up-regulating Keratin 13 Expression. J. Biol. Chem. 290, 13567-13577 (2015).
78. Rorke, E. A. et al. Structural and biochemical changes underlying a keratoderma-like phenotype in mice lacking suprabasal AP1 transcription factor function. Cell Death Dis. 6, e1647 (2015).
79. Champliaud, M. F. et al. Gene characterization of sciellin (SCEL) and protein localization in vertebrate epithelia displaying barrier properties. Genomics 70, 264-268 (2000).
80. Zhang, D. et al. Stem cell and neurogenic gene-expression profiles link prostate basal cells to aggressive prostate cancer. Nat. Commun. 7, (2016).
81. Karasawa, M. et al. Localization of metallothionein in hair follicles of normal skin and the basal cell layer of hyperplastic epidermis: Possible association with cell proliferation. J. Invest. Dermatol. 97, 97-100 (1991).
82. Beuran, M. et al. The epithelial to mesenchymal transition in pancreatic cancer: A systematic review. Pancreatology 15, 217-225 (2015).
83. Aiello, N. M. et al. EMT Subtype Influences Epithelial Plasticity and Mode of Cell Migration. Dev. Cell 45, 681-695 (2018).
84. Chen, Y.-A., Lu, I.-L. & Tsai, J.-W. Contactin-1/F3 Regulates Neuronal Migration and Morphogenesis Through Modulating RhoA Activity. Front. Mol. Neurosci. 11, 422 (2018).
85. Donato, V. Di et al. An Attractive Reelin Gradient Establishes Synaptic Lamination in the Vertebrate Visual System. Neuron 97, 1049-1062 (2018).
86. Kantor, D. B. et al. Semaphorin 5A Is a Bifunctional Axon Guidance Cue Regulated by Heparan and Chondroitin Sulfate Proteoglycans. Neuron 44, 961-975 (2004).
87. Pang, W. et al. Untangle the Multi-Facet Functions of Auts2 as an Entry Point to Understand Neurodevelopmental Disorders. Front. Psychiatry 12, 580433 (2021).
88. Hori, K. & Hoshino, M. Neuronal Migration and AUTS2 Syndrome. Brain Sci. 7, 54 (2017).
89. Sakurai, T. The role of NrCAM in neural development and disorders--beyond a simple glue in the brain. Mol. Cell. Neurosci. 49, 351-363 (2012).
90. Lau, H. H., Ng, N. H. J., Loo, L. S. W., Jasmen, J. B. & Teo, A. K. K. The molecular functions of hepatocyte nuclear factors-In and beyond the liver. J. Hepatol. 68, 1033-1048 (2018).
91. Uhlen, M. et al. Tissue-based map of the human proteome. Science (80-.). 347, 1260419 (2015).
92. Sjöstedt, E. et al. An atlas of the protein-coding genes in the human, pig, and mouse brain. Science (80-.). 367, eaay5947 (2020).
93. Robertson, A. G. et al. Comprehensive Molecular Characterization of Muscle-Invasive Bladder Cancer. Cell 171, 540-556 (2017).
94. Sjödahl, G., Eriksson, P., Liedberg, F. & Höglund, M. Molecular classification of urothelial carcinoma: global mRNA classification versus tumor-cell phenotype classification. J. Pathol. 242, 113-125 (2017).
95. Marzouka, N. A. D. et al. A validation and extended description of the Lund taxonomy for urothelial carcinoma using the TCGA cohort. Sci. Rep. 8, 3737 (2018).
96 Biankin, A. V et al. Pancreatic cancer genomes reveal aberrations in axon guidance pathway genes. Nature 491, 399-405 (2012).
97. Shi, D. D. et al. Therapeutic avenues for cancer neuroscience: translational frontiers and clinical opportunities. Lancet Oncol. In press, (2021).
98. Guo, J. A. et al. Pan-cancer Transcriptomic Predictors of Perineural Invasion Improve Occult Histopathologic Detection. Clin. Cancer Res. 27, 2807-2815 (2021).
99. Morioka, K. Degeneration of ribosomes in keratinizing cells: Possible participation of RNase A in the skin and hair of rat. Acta Histochem. Cytochem. 37, 399-406 (2004).
100. Farrell, A. S. et al. MYC regulates ductal-neuroendocrine lineage plasticity in pancreatic ductal adenocarcinoma associated with poor outcome and chemoresistance. Nat. Commun. 8, 1-12 (2017).
101. Tanaka, Y. et al. Circulation-Independent Differentiation Pathway from Extraembryonic Mesoderm toward Hematopoietic Stem Cells via Hemogenic Angioblasts. Cell Rep. 8, 31-39 (2014).
102. Komori, T. Runx2, a multifunctional transcription factor in skeletal development. J. Cell. Biochem. 87, 1-8 (2002).
103. Roel, G., Y Y, G., J, P.-M., FJ, V. & O, D. Lefl plays a role in patterning the mesoderm and ectoderm in Xenopus tropicalis. Int. J. Dev. Biol. 53, 81-89 (2009).
104. Tahara, N. et al. Sall4 regulates neuromesodermal progenitors and their descendants during body elongation in mouse embryos. Development 146, dev177659 (2019).
105. Andre, P., Song, H., Kim, W., Kispert, A. & Yang, Y. Wnt5a and Wnt11 regulate mammalian anterior-posterior axis elongation. Development 142, 1516-1527 (2015).
106. Ishikawa, A. et al. Mouse Nkd1, a Wnt antagonist, exhibits oscillatory gene expression in the PSM under the control of Notch signaling. Mech. Dev. 121, 1443-1453 (2004).
107. Cherubini, A. et al. FOXP1 circular RNA sustains mesenchymal stem cell identity via microRNA inhibition. Nucleic Acids Res. 47, 5325-5340 (2019).
108. Adams, K. L., Rousso, D. L., Umbach, J. A. & Novitch, B. G. Foxpl-mediated programming of limb-innervating motor neurons from mouse and human embryonic stem cells. Nat. Commun. 6, 1-16 (2015).
109. Sánchez-Martin, L. et al. The chemokine CXCL12 regulates monocyte-macrophage differentiation and RUNX3 expression. Blood 117, 88-97 (2011).
110. Sleightholm, R. L. et al. Emerging roles of the CXCL12/CXCR4 axis in pancreatic cancer progression and therapy. Pharmacol. Ther. 179, 158-170 (2017).
111. Cheng, H. W. et al. CCL19-producing fibroblastic stromal cells restrain lung carcinoma growth by promoting local antitumor T-cell responses. J. Allergy Clin. Immunol. 142, 1257-1271 (2018).
112. Hirth, M. et al. CXCL10 and CCL21 Promote Migration of Pancreatic Cancer Cells Toward Sensory Neurons and Neural Remodeling in Tumors in Mice, Associated With Pain in Patients. Gastroenterology 159, 665-681 (2020).
113. Boulakirba, S. et al. IL-34 and CSF-1 display an equivalent macrophage differentiation ability but a different polarization potential. Sci. Rep. 8, 256 (2018).
114. Bettac, L., Denk, S., Seufferlein, T. & Huber-Lang, M. Complement in pancreatic disease-Perpetrator or savior? Front. Immunol. 8, 15 (2017).
115. Zhang, R., Liu, Q., Li, T., Liao, Q. & Zhao, Y. Role of the complement system in the tumor microenvironment. Cancer Cell Int. 19, 300 (2019).
116. Jurcak, N. & Zheng, L. Signaling in the microenvironment of pancreatic cancer: Transmitting along the nerve. Pharmacol. Ther. 200, 126-134 (2019).
117. Puleo, F. et al. Stratification of Pancreatic Ductal Adenocarcinomas Based on Tumor and Microenvironment Features. Gastroenterology 155, 1999-2013 (2018).
118. Somerville, T. D. D. et al. Squamous trans-differentiation of pancreatic cancer cells promotes stromal inflammation. Elife 9, e53381 (2020).
119. Karamitopoulou, E. Tumor microenvironment of pancreatic cancer: immune landscape is dictated by molecular and histopathological features. Br. J. Cancer 121, 5-14 (2019).
120. Maji, S. et al. Bcl-2 Antiapoptotic Family Proteins and Chemoresistance in Cancer. Adv. Cancer Res. 137, 37-75 (2018).
121. Zhang, M. et al. Platelet-derived growth Factor D is a prognostic biomarker and is associated with platinum resistance in epithelial ovarian cancer. Int. J. Gynecol. Cancer 28, 323-331 (2018).
122. Chen, X. et al. SPP1 inhibition improves the cisplatin chemo-sensitivity of cervical cancer cell lines. Cancer Chemother. Pharmacol. 83, 603-613 (2019).
123. Wu, Q. et al. Chemoresistance to gemcitabine in hepatoma cells induces epithelial-mesenchymal transition and involves activation of PDGF-D pathway. Oncotarget 4, 1999-2009 (2013).
124. Christie, E. L. et al. Multiple ABCB1 transcriptional fusions in drug resistant high-grade serous ovarian and breast cancer. Nat. Commun. 10, 1295 (2019).
125. Wu, Z.-X. et al. Elevated ABCB1 Expression Confers Acquired Resistance to Aurora Kinase Inhibitor GSK-1070916 in Cancer Cells. Front. Pharmacol. 11, 615824 (2021).
126. Vaidyanathan, A. et al. ABCB1 (MDR1) induction defines a common resistance mechanism in paclitaxel- and olaparib-resistant ovarian cancer cells. Br. J. Cancer 115, 431-441 (2016).
127. Chauvet, S. et al. Gating of Sema3E/PlexinD1 signaling by neuropilin-1 switches axonal repulsion to attraction during brain development. Neuron 56, 807-822 (2007).
128. Wasser, C. R. & Herz, J. Reelin: Neurodevelopmental Architect and Homeostatic Regulator of Excitatory Synapses. J. Biol. Chem. 292, 1330-1338 (2017).
129. Zahalka, A. H. et al. Adrenergic nerves activate an angio-metabolic switch in prostate cancer. Science (80-.). 358, 321-326 (2017).
130. Banh, R. S. et al. Neurons release serine to support mRNA translation in pancreatic cancer. Cell 183, 1202-1218 (2020).
131. Denny, S. K. et al. Nfib Promotes Metastasis through a Widespread Increase in Chromatin Accessibility. Cell 166, 328-342 (2016).
132. Oren, Y. et al. Cycling cancer persister cells arise from lineages with distinct programs. Nat. 2021 15, 1-7 (2021).
133. Biasci, D. et al. CXCR4 inhibition in human pancreatic and colorectal cancers induces an integrated immune response. Proc. Natl. Acad. Sci. U.S.A 117, 28960-28970 (2020).
134. Feig, C. et al. Targeting CXCL12 from FAP-expressing carcinoma-associated fibroblasts synergizes with anti-PD-L1 immunotherapy in pancreatic cancer. Proc. Natl. Acad. Sci. U.S.A. 110, 20212-20217 (2013).
135. Fearon, D. T. & Janowitz, T. AMD3100/Plerixafor overcomes immune inhibition by the CXCL12-KRT19 coating on pancreatic and colorectal cancer cells. Br. J. Cancer 125, 149-151 (2021).
136. Skrypek, N. et al. The oncogenic receptor ErbB2 modulates gemcitabine and irinotecan/SN-38 chemoresistance of human pancreatic cancer cells via hCNT1 transporter and multidrug-resistance associated protein MRP-2. Oncotarget 6, 10853-10867 (2015).
137. Holmes, W. et al. Identification of heregulin, a specific activator of p185erbB2. Science (80-.). 256, 1205-1210 (1992).
138. Iwamoto, R. et al. Heparin-binding EGF-like growth factor and ErbB signaling is essential for heart function. Proc. Natl. Acad. Sci. U.S.A 100, 3221-3226 (2003).
139. Donier, E. et al. LICAM binds ErbB receptors through Ig-like domains coupling cell adhesion and neuregulin signalling. PLOS One 7, e40674 (2012).
140. Fock, V. et al. Neuregulin-1-mediated ErbB2-ErbB3 signalling protects human trophoblasts against apoptosis to preserve differentiation. J. Cell Sci. 128, 4306-4316 (2015).
141. Harder, J. et al. Multicentre phase II trial of trastuzumab and capecitabine in patients with HER2 overexpressing metastatic pancreatic cancer. Br. J. Cancer 106, 1033-1038 (2012).
142. Safran, H. et al. Lapatinib and gemcitabine for metastatic pancreatic cancer. A phase II study. Am. J. Clin. Oncol. 34, 50-52 (2011).
143. Sjoquist, K. M. et al. Personalising pancreas cancer treatment: When tissue is the issue. World J. Gastroenterol. 20, 7849-7863 (2014).
144. Chantrill, L. A. et al. Precision Medicine for Advanced Pancreas Cancer: The Individualized Molecular Pancreatic Cancer Therapy (IMPaCT) Trial. Clin. Cancer Res. 21, 2029-2037 (2015).
145. de Man, F. M., Goey, A. K. L., van Schaik, R. H. N., Mathijssen, R. H. J. & Bins, S. Individualization of Irinotecan Treatment: A Review of Pharmacokinetics, Pharmacodynamics, and Pharmacogenetics. Clin. Pharmacokinet. 2018 5710 57, 1229-1254 (2018).
146. Conroy, T. et al. FOLFIRINOX or gemcitabine as adjuvant therapy for pancreatic cancer. in New England Journal of Medicine vol. 379 2395-2406 (Massachussetts Medical Society, 2018).
147. VanDussen, K. L., Sonnek, N. M. & Stappenbeck, T. S. L-WRN conditioned medium for gastrointestinal epithelial stem cell culture shows replicable batch-to-batch activity levels across multiple research teams. Stem Cell Res. 37, 101430 (2019).
148. Boj, S. F. et al. Organoid models of human and mouse ductal pancreatic cancer. Cell 160, 324-338 (2015).
149. Freed-Pastor, W. A. et al. The CD155/TIGIT axis promotes and maintains immune evasion in neoantigen-expressing pancreatic cancer. In review, (2021).
150. Fleming, S. J., Marioni, J. C. & Babadi, M. CellBender remove-background: A deep generative model for unsupervised removal of background noise from scRNA-seq datasets. bioRxiv (2019) doi: 10.1101/791699.
151. Wolf, F. A., Angerer, P. & Theis, F. J. SCANPY: Large-scale single-cell gene expression data analysis. Genome Biol. 19, 15 (2018).
152. Korsunsky, I. et al. Fast, sensitive and accurate integration of single-cell data with Harmony. Nat. Methods 16, 1289-1296 (2019).
153. Traag, V. A., Waltman, L. & van Eck, N. J. From Louvain to Leiden: guaranteeing well-connected communities. Sci. Rep. 9, 1-12 (2019).
154. McInnes, L., Healy, J. & Melville, J. UMAP: Uniform Manifold Approximation and Projection for Dimension Reduction. arXiv (2018).
155. Büttner, M., Ostner, J., Müller, C., Theis, F. & Schubert, B. scCODA: A Bayesian model for compositional single-cell data analysis. bioRxiv 12.14.422688 (2020) doi: 10.1101/2020.12.14.422688.
156. Schapiro, D. et al. HistoCAT: Analysis of cell phenotypes and interactions in multiplex image cytometry data. Nat. Methods 14, 873-876 (2017).
157. Zanotelli, V. R. R. T. & Bodenmiller, B. ImcSegmentationPipeline: A pixelclassification based multiplexed image segmentation pipeline. (Zenodo, 2017). doi: 10.5281/zenodo.3841961.
158. Zanotelli, V., Ndamond & Strotton, M. BodenmillerGroup/ImcSegmentationPipeline: IMC Segmentation Pipeline. (2020) doi: http://doi.org/10.5281/zenodo.3841961.
159. Berg, S. et al. ilastik: interactive machine learning for (bio) image analysis. Nat. Methods 16, 1226-1232 (2019).
160. McQuin, C. et al. CellProfiler 3.0: Next-generation image processing for biology. PLOS Biol. 16, e2005970 (2018).
161. Lopez, R. et al. A joint model of unpaired data from scRNA-seq and spatial transcriptomics for imputing missing gene expression measurements. arXiv 1905.02269 (2019).
162. Muus, C. et al. Single-cell meta-analysis of SARS-COV-2 entry genes across tissues and demographics. Nat. Med. 27, 546-559 (2021).
163. Subramanian, A. et al. Gene set enrichment analysis: A knowledge-based approach for interpreting genome-wide expression profiles. Proc. Natl. Acad. Sci. U.S.A 102, 15545-15550 (2005).
164. Li, B. & Dewey, C. N. RSEM: Accurate transcript quantification from RNA-Seq data with or without a reference genome. BMC Bioinformatics 12, 323 (2011).
165. Hänzelmann, S., Castelo, R. & Guinney, J. GSVA: Gene set variation analysis for microarray and RNA-Seq data. BMC Bioinformatics 14, 7 (2013).
166. Bates, D., Mächler, M., Bolker, B. M. & Walker, S. C. Fitting linear mixed-effects models using lme4. J. Stat. Softw. 67, 1-48 (2015).
167. Kuznetsova, A., Brockhoff, P. B. & Christensen, R. H. B. ImerTest Package: Tests in Linear Mixed Effects Models. J. Stat. Softw. 82, 1-26 (2017).
168. Benjamini, Y. & Hochberg, Y. Controlling the False Discovery Rate: A Practical and Powerful Approach to Multiple Testing. J. R. Stat. Soc. Ser. B 57, 289-300 (1995).
169. Li, Q. et al. Reg proteins promote acinar-to-ductal metaplasia and act as novel diagnostic and prognostic markers in pancreatic ductal adenocarcinoma. Oncotarget 7, 77838-77853 (2016).
170. Liu, X. et al. REG3A accelerates pancreatic cancer cell growth under IL-6-associated inflammatory condition: Involvement of a REG3A-JAK2/STAT3 positive feedback loop. Cancer Lett. 362, 45-60 (2015).
171. Slebioda, T. J. et al. Triggering of TNFRSF25 promotes CD8+ T-cell responses and anti-tumor immunity. Eur. J. Immunol. 41, 2606-2611 (2011).
172. Cheng, Y., Ma, X. lei, Wei, Y. quan & Wei, X. W. Potential roles and targeted therapy of the CXCLs/CXCR2 axis in cancer and inflammatory diseases. Biochim. Biophys. Acta-Rev. Cancer 1871, 289-312 (2019).
173. Edwardson, D. W. et al. Inflammatory cytokine production in tumor cells upon chemotherapy drug exposure or upon selection for drug resistance. PLOS One 12, e0183662 (2017)
174. Tjomsland, V. et al. Interleukin la sustains the expression of inflammatory factors in human pancreatic cancer microenvironment by targeting cancer-associated fibroblasts. Neoplasia 13, 664-675 (2011).
175. He, S. et al. GFRal released by nerves enhances cancer cell perineural invasion through GDNF-RET signaling. Proc. Natl. Acad. Sci. U.S.A 111, E2008-17 (2014).
176. Mulvihill, E. E. & Drucker, D. J. Pharmacology, physiology, and mechanisms of action of dipeptidyl peptidase-4 inhibitors. Endocr. Rev. 35, 992-1019 (2014).
177. Waldmann, J. et al. Expression of neuropeptide Y and its receptors Y1 and Y2 in pancreatic intraepithelial neoplasia and invasive pancreatic cancer in a transgenic mouse model and human samples of pancreatic cancer. J. Surg. Res. 223, 230-236 (2018).
178. Vaquero, J. et al. The IGF2/IR/IGFIR Pathway in Tumor Cells and Myofibroblasts Mediates Resistance to EGFR Inhibition in Cholangiocarcinoma. Clin. Cancer Res. 24, 4282-4296 (2018).
179. Shen, R. R. et al. TRAF2 is an NF-KB activating oncogene in epithelial cancers. Oncogene 34, 209-216 (2015).
180. Sano, M. et al. Blocking CXCLs-CXCR2 axis in tumor-stromal interactions contributes to survival in a mouse model of pancreatic ductal adenocarcinoma through reduced cell invasion/migration and a shift of immune-inflammatory microenvironment. Oncogenesis 8, 8 (2019).
181. Steele, C. W. et al. CXCR2 Inhibition Profoundly Suppresses Metastases and Augments Immunotherapy in Pancreatic Ductal Adenocarcinoma. Cancer Cell 29, 832-845 (2016).
182. Liu, Q. et al. The CXCL8-CXCR1/2 pathways in cancer. Cytokine Growth Factor Rev. 31, 61-71 (2016).
183. Teijeira, A. et al. CXCR1 and CXCR2 Chemokine Receptor Agonists Produced by Tumors Induce Neutrophil Extracellular Traps that Interfere with Immune Cytotoxicity. Immunity 52, 856-871 (2020).
184. Vilgelm, A. E. & Richmond, A. Chemokines Modulate Immune Surveillance in Tumorigenesis, Metastasis, and Response to Immunotherapy. Front. Immunol. 10, 333 (2019).
185. Cavel, O. et al. Endoneurial macrophages induce perineural invasion of pancreatic cancer cells by secretion of GDNF and activation of RET tyrosine kinase receptor. Cancer Res. 72, 5733-5743 (2012).
186. Dhodapkar, M. V. Navigating the Fas lane to improved cellular therapy for cancer. J. Clin. Invest. 129, 1522-1523 (2019).
187. Shouval, D. S. et al. Interleukin 10 Receptor Signaling: Master Regulator of Intestinal Mucosal Homeostasis in Mice and Humans. Adv. Immunol. 122, 177-210 (2014).
188. Guo, Y. et al. Metabolic reprogramming of terminally exhausted CD8+ T cells by IL-10 enhances anti-tumor immunity. Nat. Immunol. 2021 226 22, 746-756 (2021).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of diagnosing, classifying and/or prognosing pancreatic ductal adenocarcinoma (PDAC), in a subject in need thereof, comprising: diagnosing the PDAC in the subject in need thereof, wherein diagnosing comprises detecting, in one or more PDAC tumor cells or organoids derived therefrom,
(i) a malignant cell signature, program or both;
(ii) a cancer-associated fibroblast (CAF) signature, program, or both;
(iii) a tumor spatial community;
(iv) one or more co-expressed receptor-ligand pairs; or
(v) any combination thereof;
wherein the diagnosing, classifying and/or prognosing the PDAC is determined based on detection of one or more of the signatures, programs, communities, or receptor-ligand pairs.

2. The method of claim 1, wherein the malignant cell signature or program comprises:
(i) a lineage specific expression program selected from a squamoid program, a mesenchymal program, a basaloid program, a classical-like program, an acinar-like program, a neuroendocrine-like program, a neural-like progenitor program, or any combination thereof;
(ii) a cell state specific expression selected from a cycling(S) program, a cycling (G2/M) program, a TNF-NFkB signaling program, a MYC signaling program, an adhesive program, a ribosomal program, an interferon signaling program, or any combination thereof;
(iii) a neoadjuvant treated malignant cell expression program;
(iv) an untreated malignant cell expression program;
(v) or any combination thereof.

3. The method of claim 2, wherein the neural-like progenitor program comprises one or more drug efflux programs and/or genes, apoptosis regulation programs and/or genes, chemoresistance programs and/or genes, tumor-nerve cross-talk programs and/or genes, neuronal gene expression programs, or neuronal development/migration/adhesion programs and/or genes, tissue stem cell module programs and/or genes, organ morphogenesis programs and/or genes, or hepatocyte nuclear factor activity programs and/or genes.

4. The method of 3, wherein the neural-like progenitor program comprises one or more genes selected from: CNTN4, CTNND2, NRXN3, RELN, SEMASA, NRCAM, AUTS2, ABCB1, BCL2, PDGFD, SPP1, SEMA3E, NFIB; any one or more genes in Table 5; any one or more genes in FIG. 15.

5. The method of claim 1, wherein the CAF signature or program comprises:
(i) a cell state specific expression program selected from an adhesive program, an immunomodulatory program, a myofibroblastic progenitor program, a neurotropic program, or any combination thereof;
(ii) a neoadjuvant treated CAF expression program;
(iii) an untreated CAF expression program; or
(iv) any combination thereof.

6. The method of claim 5, wherein the neoadjuvant treated malignant cell expression program comprises a malignant cell lineage program selected from a neural-like progenitor program, a neuroendocrine-like program, a mesenchymal program, a basaloid program, or a combination thereof; an adhesive malignant state expression program; or any combination thereof.

7. The method of claim 6, wherein the neoadjuvant treated CAF expression program comprises a fibroblast adhesive program.

8. The method of claim 1, wherein the tumor spatial community is a treatment-enriched community; a squamoid-basaloid community; or a classical community.

9. The method of claim 8, wherein the treatment-enriched community is enriched with cell(s) expressing a malignant cell lineage program selected from a neural-like progenitor program, a neuroendocrine-like program; a mesenchymal program, or an acinar-like program, or a combination thereof; cell(s) expressing a CAF expression program selected from a neurotropic program, an immunomodulatory program, or both; CD8+ T-cells; or any combination thereof.

10. The method of claim 8, wherein the squamoid-basaloid community is enriched with cell(s) expressing a malignant cell linage program selected from a squamoid program or a basaloid program, cells expressing a CAF immunomodulatory program, CD4+ T cells, B cells, regulatory T cells, natural killer cells, mast cells, conventional type 1 dendritic cells, plasmacytoid dendritic (pDC) cells, activated dendritic (aDC) cells, and/or plasma cells.

11. The method of claim 8, wherein the classical community is enriched with cell(s) expressing a CAF myofibroblastic progenitor program, cell(s) expressing a CAF adhesive program, cell(s) expressing a malignant lineage classical-like program, macrophages, conventional type 2 dendritic cells, or any combination thereof.

12. The method of claim 1, wherein the tumor spatial community is enriched in cell(s) expressing a neuroendocrine-like program and/or a neural like malignant cell lineage program, CD8+ T cells, and conventional type 2 dendritic cells.

13. The method of claim 12, wherein the tumor spatial community is depleted of conventional type 1 dendritic cells.

14. The method of claim 1, wherein the one or more co-expressed receptor-ligand pairs are selected from the pairs recited in FIG. 5B, FIG. 23, Table 3, or any combination thereof.

15. The method of claim 1, wherein the subject has had or is concurrently receiving a neoadjuvant therapy.

16. The method of claim 1, wherein the detecting comprises a single cell RNA sequencing technique or a single-nucleus RNA sequencing technique, optionally optimized for pancreatic tissue and/or for frozen tissue.

17. The method of claim 1, wherein the detecting comprises a spatially-resolved transcriptomics technique.

18. A method of treating pancreatic ductal adenocarcinoma (PDAC) in a subject in need thereof, comprising:
(a) diagnosing, classifying, and/or prognosing the PDAC in the subject in need thereof, wherein diagnosing comprises detecting, in one or more PDAC tumor cells or organoids derived therefrom,
(i) a malignant cell signature, program or both;
(ii) a cancer-associated fibroblast (CAF) signature, program, or both;
(iii) a tumor spatial community;
(iv) one or more co-expressed receptor-ligand pairs; or
(v) any combination thereof;
wherein diagnosing, classifying, and/or prognosing the PDAC is determined based on detection of one or more of the signatures, programs, communities, or receptor-ligand pairs; and
(b) administering, a PDAC treatment to the subject in need thereof, wherein the treatment comprises a PDAC malignant cell modulating agent, a CAF modulating agent, an immune modulator, an apoptosis inhibitor, a TGFbeta modulator, a CXCR4 inhibitor, a HER2 inhibitor, or any combination thereof to the subject, and
wherein the PDAC treatment administered is based at least in part on the diagnosis, classification, and/or prognosis of the PDAC.

19. The method of claim 18, wherein the immune modulator is a myeloid cell agonist or antagonist.

20. The method of claim 18, wherein the PDAC malignant cell modulating agent and/or CAF modulating agent comprise a therapeutic antibody or fragment/combination thereof, antibody-like protein scaffold, aptamer, polypeptide, a polynucleotide, a genetic modifying agent or system, a small molecule therapeutic, a chemotherapeutic, small molecule degrader, inhibitor, an immunomodulator, or any combination thereof.

21. A method of screening for one or more agents capable of treating or preventing PDAC or progression thereof comprising:
(a) contacting a PDAC tumor cell or cell population or an organoid or organoid cell population derived therefrom with a test agent or library of test agents, wherein the PDAC tumor cells or organoid cells have an initial cell state, expression signature, and/or expression program;
(b) determining a fraction of PDAC or organoid cells having a desired cell state, expression signature, and/or expression program and/or determining a fraction of PDAC or organoid cells having an undesired cell state, expression signature, and/or expression program; and
(c) selecting test agents that shift the initial PDAC or organoid cell state, expression signature, and/or expression program to a desired cell state, expression signature, and/or expression program and/or prevent a shift in the initial PDAC or organoid cell state, expression signature, and/or expression program to an undesired cell state, expression signature, and/or expression program or away from a desired cell state, expression signature, and/or expression program such that the fraction of PDAC and/or organoid cells having the desired cell state, expression signatures, and/or expression program is above a set cutoff limit.
